(19) United States
(12) Patent Application Publication
(10) Pub. No.: US 2025/0381243 A1
(43) Pub. Date: Dec. 18, 2025

(54) HETEROCYCLES AND USES THEREOF

(71) Applicant: Kumquat Biosciences Inc., San Diego, CA (US)

(72) Inventors: Pingda Ren, San Diego, CA (US); Xiaoming Li, San Diego, CA (US); Liansheng Li, San Diego, CA (US); Rasmus Hansen, San Diego, CA (US); Xiaohui He, San Diego, CA (US)

(21) Appl. No.: 19/243,961

(22) Filed: Jun. 20, 2025

Related U.S. Application Data
(63) Continuation of application No. PCT/US2023/085536, filed on Dec. 21, 2023.
(60) Provisional application No. 63/435,219, filed on Dec. 23, 2022.
(60) Provisional application No. 63/582,402, filed on Sep. 13, 2023.

(51) Int. Cl. A61K 38/17; A61K 31/517; A61K 31/5386; A61K 31/55; A61K 47/64; A61P 35/00; C07D 487/04; C07D 519/00; C07K 14/47

(52) U.S. Cl. CPC A61K 38/1709; A61K 31/517; A61K 31/5386; A61K 31/55; A61K 47/64; A61P 35/00; C07D 487/04; C07D 519/00; C07K 14/4702

(57) ABSTRACT

The present disclosure provides compounds and pharmaceutically acceptable salts thereof, and methods of using the same. The compounds and methods have a range of utilities as therapeutics, diagnostics, and research tools. In particular, the subject compositions and methods are useful for reducing signaling output of oncogenic proteins.

### CROSS-REFERENCE

This application is a continuation of International Application No. PCT/US2023/085536, filed Dec. 21, 2023, which claims the benefit of U.S. Provisional Application No. 63/435,219, filed Dec. 23, 2022, and U.S. Provisional Application No. 63/582,402, filed Sep. 13, 2023, each incorporated herein by reference in its entirety.

### SEQUENCE LISTING

The instant application contains a Sequence Listing which has been submitted electronically in XML format and is hereby incorporated by reference in its entirety. Said XML copy, created on Jun. 17, 2025, is named 56690_763_301_SL.xml and is 13,983 bytes in size.

### BACKGROUND

Cancer (e.g., tumor, neoplasm, metastases) is the second leading cause of death worldwide estimated to be responsible for about 10 million deaths each year. Many types of cancers are marked with mutations in one or more proteins involved in various signaling pathways leading to unregulated growth of cancerous cells. In some cases, about 25 to 30 percent (%) of tumors are known to harbor Rat sarcoma (Ras) mutations. In particular, mutations in the Kirsten Ras oncogene (K-Ras) are one of the most frequent Ras mutations detected in human cancers, including lung adenocarcinomas (LUADs) and pancreatic ductal adenocarcinoma (PDAC).

Ras proteins have long been considered “undruggable,” due to, in part, high affinity to their substrate guanosine-5′-triphosphate (GTP) and/or their smooth surfaces without any obvious targeting region. The specific G12C Ras gene mutation has been identified as a druggable target to which a number of G12C specific inhibitors have been developed. However, such therapeutics are still of limited application, as the G12C mutation in Ras exhibits a much lower prevalence rate as compared to other known Ras mutations, such as G12D and G12V. Drug resistance and lack of durability impose further limitations to such therapeutics.

### SUMMARY

In view of the foregoing, there remains a considerable need for a new design of therapeutics and diagnostics that can specifically target Ras, including wildtype Ras, mutants and/or associated proteins of Ras to reduce Ras signaling output. Of particular interest are inhibitors of mutant Ras proteins, such as Ras G12S and/or G12C, for the treatment of Ras-associated diseases (e.g., cancer). Such compositions and methods can be particularly useful for treating a variety of diseases including, but not limited to, cancers and neoplasia conditions. The present disclosure addresses these needs, and provides additional advantages applicable for diagnosis, prognosis, and/or treatment for a wide diversity of diseases.

In certain aspects, the present disclosure provides a modified Ras protein comprising a compound covalently bonded to one or more amino acid residues of said Ras protein, wherein the modified Ras protein comprises a compound of Formula (I′):

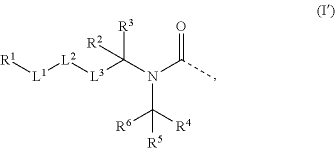

wherein:
the dashed line represents the covalent bond to the amino acid residue;
R¹ is selected from C₃₋₁₂ carbocycle and 3- to 12-membered heterocycle, each of which is optionally substituted with one or more R²⁰;
L¹ is 5- to 20-membered heterocycle optionally substituted with one or more R²⁰;
L² is selected from a bond, C₁₋₄ alkylene, 2- to 4-membered heteroalkylene, —O—, —N(R¹⁹)—, —C(O)—, —S—, —S(O)₂—, —S(O)—, —P(O)R¹⁹—, —N(R¹⁹)S(O)₂—, —N(R¹⁹)S(O)—, —N(R¹⁹)P(O)R¹⁹—, —S(O)₂N(R¹⁹)—, —S(O)N(R¹⁹)—, —P(O)R¹⁹N(R¹⁹)—, —OS(O)₂—, —OS(O)—, —OP(O)R¹⁹—, —S(O)₂O—, —S(O)O—, and —P(O)R¹⁹O—, wherein C₁₋₄ alkylene and 2- to 4-membered heteroalkylene are optionally substituted with one, two, or three R²⁰;
   L³ and R², together with the atoms to which they are attached, form 3- to 8-membered monocyclic heterocycloalkyl, C₃₋₈ monocyclic cycloalkyl, 7- to 12-membered spirocyclic heterocycloalkyl, or 7- to 12-membered fused bicyclic heterocycloalkyl, each of which is optionally substituted with one, two, or three substituents selected from halogen, —OH, —CN, C₁₋₆ alkyl, C₃₋₆ cycloalkyl, —O(C₁₋₆ alkyl), and —O(C₃₋₆ cycloalkyl), wherein C₁₋₆ alkyl, C₃₋₆ cycloalkyl, —O(C₁₋₆ alkyl), and —O(C₃₋₆ cycloalkyl) are optionally substituted with one, two, or three substituents selected from halogen, —OH, —CN, C₁₋₃ alkyl, C₁₋₃ haloalkyl, C₃₋₆ cycloalkyl, —O(C₁₋₃ alkyl), and —O(C₁₋₃ haloalkyl);
   L³ and R⁶, together with the atoms to which they are attached, form 4- to 8-membered monocyclic heterocycloalkyl, 7- to 12-membered spirocyclic heterocycloalkyl, or 7- to 12-membered fused bicyclic heterocycloalkyl, each of which is optionally substituted with one, two, or three substituents selected from halogen, —OH, —CN, C₁₋₆ alkyl, C₃₋₆ cycloalkyl, —O(C₁₋₆ alkyl), and —O(C₃₋₆ cycloalkyl), wherein C₁₋₆ alkyl, C₃₋₆ cycloalkyl, —O(C₁₋₆ alkyl), and —O(C₃₋₆ cycloalkyl) are optionally substituted with one, two, or three substituents selected from halogen, —OH, —CN, C₁₋₃ alkyl, C₁₋₃ haloalkyl, C₃₋₆ cycloalkyl, —O(C₁₋₃ alkyl), and —O(C₁₋₃ haloalkyl), and wherein the 4- to 8-membered monocyclic heterocycloalkyl formed by L³ and R⁶ is not piperazine; or
   R² and R⁶, together with the atoms to which they are attached, form 3- to 8-membered monocyclic heterocycloalkyl optionally substituted with one, two, or three substituents selected from halogen, —OH, —CN, C₁₋₆ alkyl, C₃₋₆ cycloalkyl, —O(C₁₋₆ alkyl), and —O(C₃₋₆ cycloalkyl), wherein C₁₋₆ alkyl, C₃₋₆ cycloalkyl, —O(C₁₋₆ alkyl), and —O(C₃₋₆ cycloalkyl) are optionally substituted with one, two, or three substituents selected from halogen, —OH, —CN, C₁₋₃ alkyl, C₁₋₃ haloalkyl, C₃₋₆ cycloalkyl, —O(C₁₋₃ alkyl), and —O(C₁₋₃ haloalkyl), and wherein L³ is a bond;
R² is selected from halogen, —CN, C₁₋₆ alkyl, and C₃₋₆ cycloalkyl, or R² and R³, together with the carbon atom to which they are attached, form C₃₋₆ cycloalkyl, wherein C₁₋₆ alkyl and C₃₋₆ cycloalkyl are optionally substituted with one, two, or three substituents selected from halogen, —CN, C₁₋₆ alkyl, C₁₋₆ haloalkyl, C₃₋₆ cycloalkyl, —O(C₁₋₆ alkyl), and —O(C₁₋₆ haloalkyl);
R³ is selected from hydrogen, halogen, —CN, C₁₋₆ alkyl, and C₃₋₆ cycloalkyl, wherein C₁₋₆ alkyl and C₃₋₆ cycloalkyl are optionally substituted with one, two, or three substituents selected from halogen, —CN, C₁₋₆ alkyl, C₁₋₆ haloalkyl, C₃₋₆ cycloalkyl, —O(C₁₋₆ alkyl), and —O(C₁₋₆ haloalkyl);
R⁴, R⁵, and R⁶ are each independently selected from hydrogen, halogen, —CN, C₁₋₆ alkyl, and C₃₋₆ cycloalkyl, or R⁴ and R⁵, together with the carbon atom to which they are attached, form C₃₋₆ cycloalkyl, wherein C₁₋₆ alkyl and C₃₋₆ cycloalkyl are optionally substituted with one, two, or three substituents selected from halogen, —CN, C₁₋₆ alkyl, C₁₋₆ haloalkyl, C₃₋₆ cycloalkyl, —O(C₁₋₆ alkyl), and —O(C₁₋₆ haloalkyl);

R¹⁹ is independently selected at each occurrence from hydrogen, —CN, C₁₋₆ alkyl, C₃₋₆ carbocycle, 3- to 6-membered heterocycle, —OH, —O(C₁₋₆ alkyl), and —O(C₁₋₆ haloalkyl);

R²⁰ is independently selected at each occurrence from halogen, oxo, —CN, C₁₋₆ alkyl, C₂₋₆ alkenyl, C₂₋₆ alkynyl, 2- to 6-membered heteroalkyl, 2- to 6-membered heteroalkenyl, 2- to 6-membered heteroalkynyl, —C₀₋₆ alkyl-(C₃₋₁₂ carbocycle), -(2- to 6-membered heteroalkyl)-(C₃₋₁₂ carbocycle), —C₀₋₆ alkyl-(3- to 12-membered heterocycle), -(2- to 6-membered heteroalkyl)-(3- to 12-membered heterocycle), —OR²², —SR²², —N(R²²)(R²³), —NR²², —C(R²¹)₂, —C(O)OR²², —OC(O)N(R²²)(R²³), —N(R²²)C(O)N(R²²)(R²³), —N(R²²)C(O)OR²², —N(R²²)S(O)₂R²², —C(O)R²², —S(O)R²², —OC(O)R²², —C(O)N(R²²)(R²³), —C(O)C(O)N(R²²)(R²³), —N(R²²)C(O)R²², —S(O)₂R²², —S(O)(NR²²)R²², —S(O)₂N(R²²)(R²³)—, —S(═O)(═NR²²)N(R²²)(R²³), and —OCH₂C(O)OR²²; wherein two R²⁰ attached to the same or adjacent atoms optionally join to form C₃₋₁₂ carbocycle or 3- to 12-membered heterocycle; wherein C₁₋₆ alkyl, C₂₋₆ alkenyl, C₂₋₆ alkynyl, 2- to 6-membered heteroalkyl, 2- to 6-membered heteroalkenyl, 2- to 6-membered heteroalkynyl, —C₀₋₆ alkyl-(C₃₋₁₂ carbocycle), —(2- to 6-membered heteroalkyl)-(C₃₋₁₂ carbocycle), —C₀₋₆ alkyl-(3- to 12-membered heterocycle), -(2- to 6-membered heteroalkyl)-(3- to 12-membered heterocycle), C₃₋₁₂ carbocycle, and 3- to 12-membered heterocycle are optionally substituted with one or more substituents independently selected from halogen, oxo, —CN, C₁₋₆ alkyl, C₁₋₆ haloalkyl, C₁₋₆ alkoxy, C₁₋₆ haloalkoxy, —OR²², —SR²², —N(R²²)(R²³), ═NR²², ═C(R²¹)₂, —C(O)OR²², —OC(O)N(R²²)(R²³), —N(R²²)C(O)N(R²²)(R²³), —N(R²²)C(O)OR²², —N(R²²)S(O)₂R²², —C(O)R²², —S(O)R²², —OC(O)R²², —C(O)N(R²²)(R²³), —C(O)C(O)N(R²²)(R²³), —N(R²²)C(O)R²², —S(O)₂R²², —S(O)(NR²²)R²², —S(O)₂N(R²²)(R²³), and —S(═O)(—NR²²)N(R²²)(R²³);
R²¹ is independently selected at each occurrence from hydrogen, halogen, C₁₋₆ alkyl, C₁₋₆ haloalkyl, —C₀₋₆ alkyl-(C₃₋₁₂ carbocycle), and —C₀₋₆ alkyl-(3- to 12-membered heterocycle), or two R²¹ are taken together with the carbon atom to which they are attached to form C₃₋₁₂ carbocycle or 3- to 12-membered heterocycle, each of which is optionally substituted with one, two, or three substituents independently selected from halogen, C₁₋₃ alkyl, C₁₋₃ haloalkyl, and —OH;
R²² is independently selected at each occurrence from hydrogen, C₁₋₆ alkyl, C₁₋₆ haloalkyl, —C₀₋₆ alkyl-(C₃₋₁₂ carbocycle), and —C₀₋₆ alkyl-(3- to 12-membered heterocycle); and
R²³ is independently selected at each occurrence from hydrogen and C₁₋₆ alkyl; or R²² and R²³ attached to the same nitrogen atom form 3- to 10 membered heterocycle.

In some embodiments, the modified Ras protein is a modified human K-Ras mutant protein comprising a compound covalently bonded to a serine residue having the structure of Formula (I), wherein the serine residue corresponds to position 12 of SEQ ID No. 4:

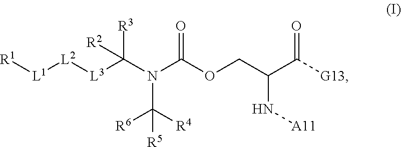

and wherein the dashed lines represent bonds between the serine residue and alanine 11 and glycine 13 of the K-Ras mutant protein, respectively.

In some embodiments, the modified protein of Formula (I′) or (I) exhibits a reduced Ras signaling output. The reduced Ras signaling output may be evidenced by one or more output selected from (i) an increase in steady state level of GDP-bound modified protein; (ii) a reduction in steady state level of GTP-bound modified protein; (iii) a reduction of phosphorylated AKTs473; (iv) a reduction of phosphorylated ERK T202/Y204; (v) a reduction of phosphorylated S6 S235/236; (vi) a reduction of cell growth of a tumor cell expressing a Ras G12S mutant protein; and (vii) a reduction in Ras interaction with a Ras-pathway signaling protein.

In some embodiments, the modified protein of Formula (I′) or (I) comprises an amino acid sequence in SEQ ID No. 4 having the serine residue corresponding to position 12 of SEQ ID No. 1. In some embodiments, the modified protein comprises an amino acid sequence of SEQ ID No. 4.

In some embodiments, the modified protein of Formula (I′) or (I) is formed by contacting a precursor compound with the serine residue of an unmodified Ras G12S mutant protein, wherein the precursor compound comprises a staying group and a leaving group, and wherein said contacting results in release of the leaving group and formation of said modified protein. In some embodiments, the precursor compound is a compound described herein, such as a compound of Formula (II), (II-a), (II-b), (II-c), or (II-d). In some embodiments, the modified protein comprises an amino acid sequence in SEQ ID No. 1 having the serine residue corresponding to position 12 of SEQ ID No. 1, and wherein the precursor compound selectively labels the serine residue as compared to (i) an aspartate residue of a K-Ras G12D mutant protein, said aspartate corresponding to position 12 of SEQ ID No. 2; (ii) a valine residue of a K-Ras G12V mutant protein, said valine corresponding to position 12 of SEQ ID No. 3; and/or (iii) a glycine residue of a K-Ras wildtype protein, said glycine corresponding to position 12 of SEQ ID No. 1. In some embodiments, the precursor compound selectively labels the serine residue by at least 2-fold, 3-fold, 4-fold, 5-fold, or more when assayed under comparable conditions. In some embodiments, the contacting occurs in vitro. In some embodiments, the contacting occurs in vivo. In some embodiments, the leaving group is selected from

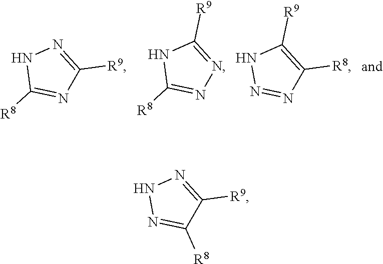

or a salt or tautomer thereof, wherein R⁸ and R⁹ are each independently selected from hydrogen, halogen, —CN, C₁₋₆ alkyl, and C₃₋₆ cycloalkyl, wherein C₁₋₆ alkyl and C₃₋₆ cycloalkyl are optionally substituted with one, two, or three substituents selected from halogen, —CN, C₁₋₆ alkyl, —O(C₁₋₆ alkyl), and —O(C₁₋₆ haloalkyl).

In certain aspects, the present disclosure provides a compound of Formula (II):

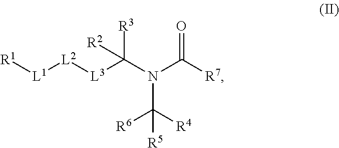

or a pharmaceutically acceptable salt or solvate thereof, wherein:
R¹ is selected from C₃₋₁₂ carbocycle and 3- to 12-membered heterocycle, each of which is optionally substituted with one or more R²⁰;
L¹ is 5- to 20-membered heterocycle optionally substituted with one or more R²⁰;
L² is selected from a bond, C₁₋₄ alkylene, 2- to 4-membered heteroalkylene, —O—, —N(R¹⁹)—, —C(O)—, —S—, —S(O)₂—, —S(O)—, —P(O)R¹⁹—, —N(R¹⁹)S(O)₂—, —N(R¹⁹)S(O)—, —N(R¹⁹)P(O)R¹⁹—, —S(O)₂N(R¹⁹)—, —S(O)N(R¹⁹)—, —P(O)R¹⁹N(R¹⁹)—, —OS(O)₂—, —OS(O)—, —OP(O)R¹⁹—, —S(O)₂O—, —S(O)O—, and —P(O)R¹⁹O—, wherein C₁₋₄ alkylene and 2- to 4-membered heteroalkylene are optionally substituted with one, two, or three R²⁰;
   L³ and R², together with the atoms to which they are attached, form 3- to 8-membered monocyclic heterocycloalkyl, C₃₋₈ monocyclic cycloalkyl, 7- to 12-membered spirocyclic heterocycloalkyl, or 7- to 12-membered fused bicyclic heterocycloalkyl, each of which is optionally substituted with one, two, or three substituents selected from halogen, —OH, —CN, C₁₋₆ alkyl, C₃₋₆ cycloalkyl, —O(C₁₋₆ alkyl), and —O(C₃₋₆ cycloalkyl), wherein C₁₋₆ alkyl, C₃₋₆ cycloalkyl, —O(C₁₋₆ alkyl), and —O(C₃₋₆ cycloalkyl) are optionally substituted with one, two, or three substituents selected from halogen, —OH, —CN, C₁₋₃ alkyl, C₁₋₃ haloalkyl, C₃₋₆ cycloalkyl, —O(C₁₋₃ alkyl), and —O(C₁₋₃ haloalkyl);
   L³ and R⁶, together with the atoms to which they are attached, form 4- to 8-membered monocyclic heterocycloalkyl, 7- to 12-membered spirocyclic heterocycloalkyl, or 7- to 12-membered fused bicyclic heterocycloalkyl, each of which is optionally substituted with one, two, or three substituents selected from halogen, —OH, —CN, C₁₋₆ alkyl, C₃₋₆ cycloalkyl, —O(C₁₋₆ alkyl), and —O(C₃₋₆ cycloalkyl), wherein C₁₋₆ alkyl, C₃₋₆ cycloalkyl, —O(C₁₋₆ alkyl), and —O(C₃₋₆ cycloalkyl) are optionally substituted with one, two, or three substituents selected from halogen, —OH, —CN, C₁₋₃ alkyl, C₁₋₃ haloalkyl, C₃₋₆ cycloalkyl, —O(C₁₋₃ alkyl), and —O(C₁₋₃ haloalkyl), and wherein the 4- to 8-membered monocyclic heterocycloalkyl formed by L³ and R⁶ is not piperazine; or
   R² and R⁶, together with the atoms to which they are attached, form 3- to 8-membered monocyclic heterocycloalkyl optionally substituted with one, two, or three substituents selected from halogen, —OH, —CN, C₁₋₆ alkyl, C₃₋₆ cycloalkyl, —O(C₁₋₆ alkyl), and —O(C₃₋₆ cycloalkyl), wherein C₁₋₆ alkyl, C₃₋₆ cycloalkyl, —O(C₁₋₆ alkyl), and —O(C₃₋₆ cycloalkyl) are optionally substituted with one, two, or three substituents selected from halogen, —OH, —CN, C₁₋₃ alkyl, C₁₋₃ haloalkyl, C₃₋₆ cycloalkyl, —O(C₁₋₃ alkyl), and —O(C₁₋₃ haloalkyl), and wherein L³ is a bond;
R² is selected from halogen, —CN, C₁₋₆ alkyl, and C₃₋₆ cycloalkyl, or R² and R³, together with the carbon atom to which they are attached, form C₃₋₆ cycloalkyl, wherein C₁₋₆ alkyl and C₃₋₆ cycloalkyl are optionally substituted with one, two, or three substituents selected from halogen, —CN, C₁₋₆ alkyl, C₁₋₆ haloalkyl, C₃₋₆ cycloalkyl, —O(C₁₋₆ alkyl), and —O(C₁₋₆ haloalkyl);
R³ is selected from hydrogen, halogen, —CN, C₁₋₆ alkyl, and C₃₋₆ cycloalkyl, wherein C₁₋₆ alkyl and C₃₋₆ cycloalkyl are optionally substituted with one, two, or three substituents selected from halogen, —CN, C₁₋₆ alkyl, C₁₋₆ haloalkyl, C₃₋₆ cycloalkyl, —O(C₁₋₆ alkyl), and —O(C₁₋₆ haloalkyl);
R⁴, R⁵, and R⁶ are each independently selected from hydrogen, halogen, —CN, C₁₋₆ alkyl, and C₃₋₆ cycloalkyl, or R⁴ and R⁵, together with the carbon atom to which they are attached, form C₃₋₆ cycloalkyl, wherein C₁₋₆ alkyl and C₃₋₆ cycloalkyl are optionally substituted with one, two, or three substituents selected from halogen, —CN, C₁₋₆ alkyl, C₁₋₆ haloalkyl, C₃₋₆ cycloalkyl, —O(C₁₋₆ alkyl), and —O(C₁₋₆ haloalkyl);
R⁷ is selected from

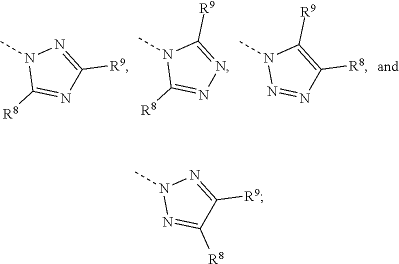

R⁸ and R⁹ are each independently selected from hydrogen, halogen, —CN, C₁₋₆ alkyl, and C₃₋₆ cycloalkyl, wherein C₁₋₆ alkyl and C₃₋₆ cycloalkyl are optionally substituted with one, two, or three substituents selected from halogen, —CN, C₁₋₆ alkyl, —O(C₁₋₆ alkyl), and —O(C₁₋₆ haloalkyl);

R¹⁹ is independently selected at each occurrence from hydrogen, —CN, C₁₋₆ alkyl, C₃₋₆ carbocycle, 3- to 6-membered heterocycle, —OH, —O(C₁₋₆ alkyl), and —O(C₁₋₆ haloalkyl);

R²⁰ is independently selected at each occurrence from halogen, oxo, —CN, C₁₋₆ alkyl, C₂₋₆ alkenyl, C₂₋₆ alkynyl, 2- to 6-membered heteroalkyl, 2- to 6-membered heteroalkenyl, 2- to 6-membered heteroalkynyl, —C₀₋₆ alkyl-(C₃₋₁₂ carbocycle), -(2- to 6-membered heteroalkyl)-(C₃₋₁₂ carbocycle), —C₀₋₆ alkyl-(3- to 12-membered heterocycle), -(2- to 6-membered heteroalkyl)-(3- to 12-membered heterocycle), —OR²², —SR²², —N(R²²)(R²³), —NR²², ═C(R²¹)₂, —C(O)OR²², —OC(O)N(R²²)(R²³), —N(R²²)C(O)N(R²²)(R²³), —N(R²²)C(O)OR²², —N(R²²)S(O)₂R²², —C(O)R²², —S(O)R²², —OC(O)R²², —C(O)N(R²²)(R²³), —C(O)C(O)N(R²²)(R²³), —N(R²²)C(O)R²², —S(O)₂R²², —S(O)(NR²²)R²², —S(O)₂N(R²²)(R²³)—, —S(—OX—NR²²)N(R²²)(R²³), and —OCH₂C(O)OR²²; wherein two R²⁰ attached to the same or adjacent atoms optionally join to form C₃₋₁₂ carbocycle or 3- to 12-membered heterocycle; wherein C₁₋₆ alkyl, C₂₋₆ alkenyl, C₂₋₆ alkynyl, 2- to 6-membered heteroalkyl, 2- to 6-membered heteroalkenyl, 2- to 6-membered heteroalkynyl, —C₀₋₆ alkyl-(C₃₋₁₂ carbocycle), -(2- to 6-membered heteroalkyl)-(C₃₋₁₂ carbocycle), —C₀₋₆ alkyl-(3- to 12-membered heterocycle), -(2- to 6-membered heteroalkyl)-(3- to 12-membered heterocycle), C₃₋₁₂ carbocycle, and 3- to 12-membered heterocycle are optionally substituted with one or more substituents independently selected from halogen, oxo, —CN, C₁₋₆ alkyl, C₁₋₆ haloalkyl, C₁₋₆ alkoxy, C₁₋₆ haloalkoxy, —OR²², —SR²², —N(R²²)(R²³), ═NR²², ═C(R²¹)₂, —C(O)OR²², —OC(O)N(R²²)(R²³), —N(R²²)C(O)N(R²²)(R²³), —N(R²²)C(O)OR²², —N(R²²)S(O)₂R²², —C(O)R²², —S(O)R²², —OC(O)R²², —C(O)N(R²²)(R²³), —C(O)C(O)N(R²²)(R²³), —N(R²²)C(O)R²², —S(O)₂R²², —S(O)(NR²²)R²², —S(O)₂N(R²²)(R²³), and —S(═O)(═NR²²)N(R²²)(R²³);

R²¹ is independently selected at each occurrence from hydrogen, halogen, C₁₋₆ alkyl, C₁₋₆ haloalkyl, —C₀₋₆ alkyl-(C₃₋₁₂ carbocycle), and —C₀₋₆ alkyl-(3- to 12-membered heterocycle), or two R²¹ are taken together with the carbon atom to which they are attached to form C₃₋₁₂ carbocycle or 3- to 12-membered heterocycle, each of which is optionally substituted with one, two, or three substituents independently selected from halogen, C₁₋₃ alkyl, C₁₋₃ haloalkyl, and —OH;

R²² is independently selected at each occurrence from hydrogen, C₁₋₆ alkyl, C₁₋₆ haloalkyl, —C₀₋₆ alkyl-(C₃₋₁₂ carbocycle), and —C₀₋₆ alkyl-(3- to 12-membered heterocycle); and

R²³ is independently selected at each occurrence from hydrogen and C₁₋₆ alkyl; or R²² and R²³ attached to the same nitrogen atom form 3- to 10 membered heterocycle.

In some embodiments, a subject compound of any formulae disclosed herein, including a compound of Formula (II), (II-a), (II-b), (II-c), or (II-d), reversibly binds to a K-Ras protein with an IC₅₀ of less than 1000 nM as assessed by an HTRF assay when R⁷ is replaced with hydrogen. In some embodiments, a subject compound of any formulae disclosed herein, including a compound of Formula (II), (II-a), (II-b), (II-c), or (II-d), reversibly binds to the Switch II pocket of a Ras protein.

In some embodiments, for a compound or modified protein or the present disclosure, L³ and R², together with the atoms to which they are attached, form 3- to 8-membered monocyclic heterocycloalkyl, C₃₋₈ monocyclic cycloalkyl, 7- to 12-membered spirocyclic heterocycloalkyl, or 7- to 12-membered fused bicyclic heterocycloalkyl, each of which is optionally substituted with one, two, or three substituents selected from halogen, —OH, —CN, C₁₋₆ alkyl, C₃₋₆ cycloalkyl, —O(C₁₋₆ alkyl), and —O(C₃₋₆ cycloalkyl), wherein C₁₋₆ alkyl, C₃₋₆ cycloalkyl, —O(C₁₋₆ alkyl), and —O(C₃₋₆ cycloalkyl) are optionally substituted with one, two, or three substituents selected from halogen, —OH, —CN, C₁₋₃ alkyl, C₁₋₃ haloalkyl, C₃₋₆ cycloalkyl, —O(C₁₋₃ alkyl), and —O(C₁₋₃ haloalkyl). In some embodiments, L³ and R², together with the atoms to which they are attached, form 3- to 8-membered monocyclic heterocycloalkyl optionally substituted with one, two, or three substituents selected from halogen, —OH, —CN, C₁₋₆ alkyl, C₃₋₆ cycloalkyl, —O(C₁₋₆ alkyl), and —O(C₃₋₆ cycloalkyl), wherein C₁₋₆ alkyl, C₃₋₆ cycloalkyl, —O(C₁₋₆ alkyl), and —O(C₃₋₆ cycloalkyl) are optionally substituted with one, two, or three substituents selected from halogen, —OH, —CN, C₁₋₃ alkyl, C₁₋₃ haloalkyl, C₃₋₆ cycloalkyl, —O(C₁₋₃ alkyl), and —O(C₁₋₃ haloalkyl). In some embodiments, L³ and R⁶, together with the atoms to which they are attached, form 4- to 8-membered monocyclic heterocycloalkyl, 7- to 12-membered spirocyclic heterocycloalkyl, or 7- to 12-membered fused bicyclic heterocycloalkyl, each of which is optionally substituted with one, two, or three substituents selected from halogen, —OH, —CN, C₁₋₆ alkyl, C₃₋₆ cycloalkyl, —O(C₁₋₆ alkyl), and —O(C₃₋₆ cycloalkyl), wherein C₁₋₆ alkyl, C₃₋₆ cycloalkyl, —O(C₁₋₆ alkyl), and —O(C₃₋₆ cycloalkyl) are optionally substituted with one, two, or three substituents selected from halogen, —OH, —CN, C₁₋₃ alkyl, C₁₋₃ haloalkyl, C₃₋₆ cycloalkyl, —O(C₁₋₃ alkyl), and —O(C₁₋₃ haloalkyl), and wherein the 4- to 8-membered monocyclic heterocycloalkyl formed by L³ and R⁶ is not piperazine. In some embodiments, R² and R⁶, together with the atoms to which they are attached, form 3- to 8-membered monocyclic heterocycloalkyl optionally substituted with one, two, or three substituents selected from halogen, —OH, —CN, C₁₋₆ alkyl, C₃₋₆ cycloalkyl, —O(C₁₋₆ alkyl), and —O(C₃₋₆ cycloalkyl), wherein C₁₋₆ alkyl, C₃₋₆ cycloalkyl, —O(C₁₋₆ alkyl), and —O(C₃₋₆ cycloalkyl) are optionally substituted with one, two, or three substituents selected from halogen, —OH, —CN, C₁₋₃ alkyl, C₁₋₃ haloalkyl, C₃₋₆ cycloalkyl, —O(C₁₋₃ alkyl), and —O(C₁₋₃ haloalkyl), and wherein L³ is a bond.

In some embodiments, the compound of Formula (II) is a compound of Formula (II-a):

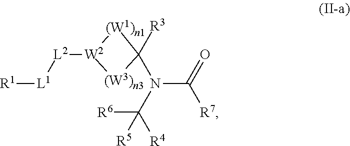

or a pharmaceutically acceptable salt or solvate thereof, wherein:
W¹ and W³ are each independently selected from N(R¹⁰), C(R¹¹)₂, C(O), O, S(O), and S(O)₂;
W² is selected from N and C(R¹¹);
n1 and n3 are each independently selected from 0, 1, 2, 3, 4, and 5, wherein the sum of n1 and n3 is at least 1;
R¹⁰ is independently selected at each occurrence from hydrogen, C₁₋₆ alkyl, and C₃₋₆ cycloalkyl, wherein C₁₋₆ alkyl and C₃₋₆ cycloalkyl are optionally substituted with one, two, or three substituents selected from halogen, —OH, —CN, C₁₋₃ alkyl, C₁₋₃ haloalkyl, C₃₋₆ cycloalkyl, —O(C₁₋₃ alkyl), and —O(C₁₋₃ haloalkyl); and
R¹¹ is independently selected at each occurrence from hydrogen, halogen, —OH, —CN, C₁₋₆ alkyl, C₃₋₆ cycloalkyl, —O(C₁₋₆ alkyl), and —O(C₃₋₆ cycloalkyl), or two R¹¹ attached to the same carbon atom form C₃₋₆ cycloalkyl, wherein C₁₋₆ alkyl, C₃₋₆ cycloalkyl, —O(C₁₋₆ alkyl), and —O(C₃₋₆ cycloalkyl) are optionally substituted with one, two, or three substituents selected from halogen, —OH, —CN, C₁₋₃ alkyl, C₁₋₃ haloalkyl, C₃₋₆ cycloalkyl, —O(C₁₋₃ alkyl), and —O(C₁₋₃ haloalkyl).

In some embodiments, for a compound of Formula (II-a), W¹ and W³ are each C(R¹¹)₂. In some embodiments, W² is N. In some embodiments, the sum of n1 and n3 is 2, 3, 4 or 5. In some embodiments, n1 is 2, 3, or 4 and n3 is 1 or 2.

In some embodiments, the compound of Formula (II) is a compound of Formula (II-b):

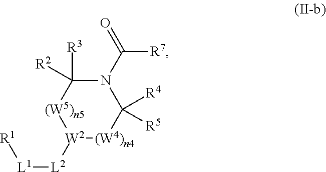

or a pharmaceutically acceptable salt or solvate thereof, wherein:
W⁴ and W⁵ are each independently selected from N(R¹⁰), C(R¹¹)₂, C(O), O, S(O), and S(O)₂;
W² is selected from N and C(R¹¹);
n4 and n5 are each independently selected from 0, 1, 2, 3, 4, and 5;
R¹⁰ is independently selected at each occurrence from hydrogen, C₁₋₆ alkyl, and C₃₋₆ cycloalkyl, wherein C₁₋₆ alkyl and C₃₋₆ cycloalkyl are optionally substituted with one, two, or three substituents selected from halogen, —OH, —CN, C₁₋₃ alkyl, C₁₋₃ haloalkyl, C₃₋₆ cycloalkyl, —O(C₁₋₃ alkyl), and —O(C₁₋₃ haloalkyl); and
R¹¹ is independently selected at each occurrence from hydrogen, halogen, —OH, —CN, C₁₋₆ alkyl, C₃₋₆ cycloalkyl, —O(C₁₋₆ alkyl), and —O(C₃₋₆ cycloalkyl), or two R¹¹ attached to the same carbon atom form C₃₋₆ cycloalkyl, wherein C₁₋₆ alkyl, C₃₋₆ cycloalkyl, —O(C₁₋₆ alkyl), and —O(C₃₋₆ cycloalkyl) are optionally substituted with one, two, or three substituents selected from halogen, —OH, —CN, C₁₋₃ alkyl, C₁₋₃ haloalkyl, C₃₋₆ cycloalkyl, —O(C₁₋₃ alkyl), and —O(C₁₋₃ haloalkyl).

In some embodiments, for a compound of Formula (II-b), W⁴ and W⁵ are each C(R¹¹)₂. In some embodiments, W² is C(R¹¹). In some embodiments, the sum of n4 and n5 is 0 or 1. In some embodiments, n4 and n5 are each 0.

In some embodiments, the compound of Formula (II) is a compound of Formula (II-c):

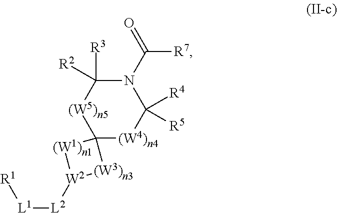

or a pharmaceutically acceptable salt or solvate thereof, wherein:
W¹, W³, W⁴, and W⁵ are each independently selected from N(R¹⁰), C(R¹¹)₂, C(O), O, S(O), and S(O)₂;
W² is selected from N and C(R¹¹);
n1 and n3 are each independently selected from 0, 1, 2, 3, 4, and 5, wherein the sum of n1 and n3 is at least 2;
n4 and n5 are each independently selected from 0, 1, 2, 3, 4, and 5;
R¹⁰ is independently selected at each occurrence from hydrogen, C₁₋₆ alkyl, and C₃₋₆ cycloalkyl, wherein C₁₋₆ alkyl and C₃₋₆ cycloalkyl are optionally substituted with one, two, or three substituents selected from halogen, —OH, —CN, C₁₋₃ alkyl, C₁₋₃ haloalkyl, C₃₋₆ cycloalkyl, —O(C₁₋₃ alkyl), and —O(C₁₋₃ haloalkyl); and
R¹¹ is independently selected at each occurrence from hydrogen, halogen, —OH, —CN, C₁₋₆ alkyl, C₃₋₆ cycloalkyl, —O(C₁₋₆ alkyl), and —O(C₃₋₆ cycloalkyl), or two R¹¹ attached to the same carbon atom form C₃₋₆ cycloalkyl, wherein C₁₋₆ alkyl, C₃₋₆ cycloalkyl, —O(C₁₋₆ alkyl), and —O(C₃₋₆ cycloalkyl) are optionally substituted with one, two, or three substituents selected from halogen, —OH, —CN, C₁₋₃ alkyl, C₁₋₃ haloalkyl, C₃₋₆ cycloalkyl, —O(C₁₋₃ alkyl), and —O(C₁₋₃ haloalkyl).

In some embodiments, for a compound of Formula (II-c), each W¹ is independently selected from C(R¹¹)₂ and O. In some embodiments, n1 is 2, 3, or 4, one W¹ is O, and the remaining W¹ are each C(R¹¹)₂. In some embodiments, W³ is C(R¹¹)₂. In some embodiments, W² is N. In some embodiments, W⁴ and W⁵ are each C(R¹¹)₂. In some embodiments, the sum of n1 and n3 is 2, 3, 4, or 5. In some embodiments, n1 is 2, 3, or 4 and n3 is 1. In some embodiments, the sum of n4 and n5 is 0 or 1. In some embodiments, n4 and n5 are each 0. In some embodiments.

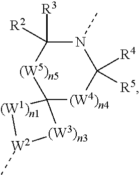

is selected from

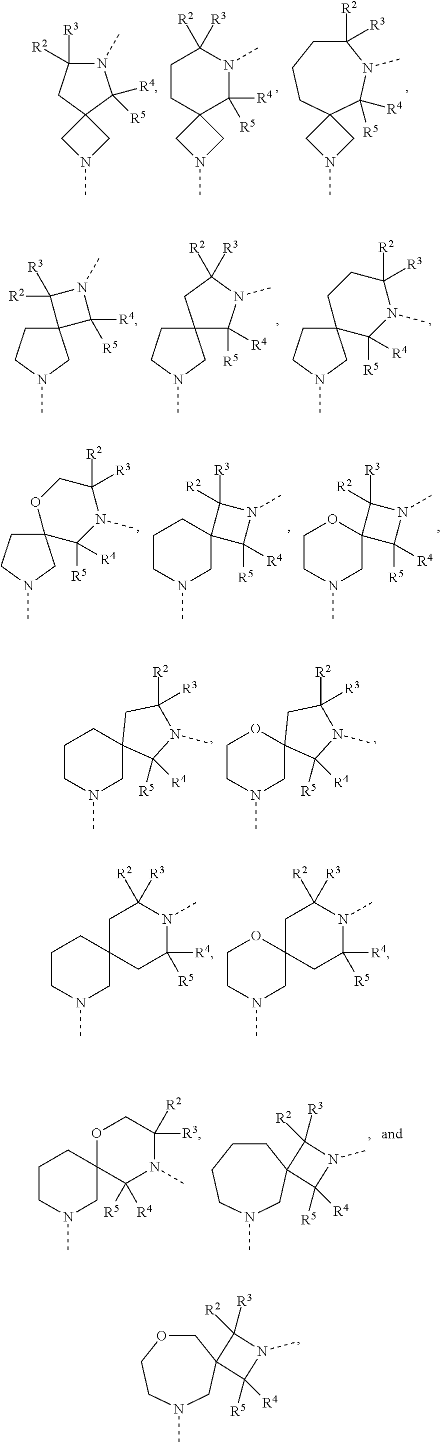

each of which is optionally substituted with one, two, or three R¹¹.

In some embodiments, the compound of Formula (II) is a compound of Formula (II-d):

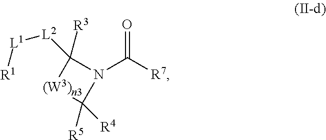

or a pharmaceutically acceptable salt or solvate thereof, wherein:
W³ is selected from N(R¹⁰), C(R¹¹)₂, C(O), O, S(O), and S(O)₂;
n3 is selected from 0, 1, 2, 3, 4, and 5;
R¹⁰ is independently selected at each occurrence from hydrogen, C₁₋₆ alkyl, and C₃₋₆ cycloalkyl, wherein C₁₋₆ alkyl and C₃₋₆ cycloalkyl are optionally substituted with one, two, or three substituents selected from halogen, —OH, —CN, C₁₋₃ alkyl, C₁₋₃ haloalkyl, C₃₋₆ cycloalkyl, —O(C₁₋₃ alkyl), and —O(C₁₋₃ haloalkyl); and
R¹¹ is independently selected at each occurrence from hydrogen, halogen, —OH, —CN, C₁₋₆ alkyl, C₃₋₆ cycloalkyl, —O(C₁₋₆ alkyl), and —O(C₃₋₆ cycloalkyl), or two R¹¹ attached to the same carbon atom form C₃₋₆ cycloalkyl, wherein C₁₋₆ alkyl, C₃₋₆ cycloalkyl, —O(C₁₋₆ alkyl), and —O(C₃₋₆ cycloalkyl) are optionally substituted with one, two, or three substituents selected from halogen, —OH, —CN, C₁₋₃ alkyl, C₁₋₃ haloalkyl, C₃₋₆ cycloalkyl, —O(C₁₋₃ alkyl), and —O(C₁₋₃ haloalkyl).

In some embodiments, for a compound of Formula (II-d), each W³ is C(R¹¹)₂. In some embodiments, n3 is 1, 2, or 3.

In some embodiments, for a compound of Formula (II), (II-a), (II-b), (II-c), or (II-d), R¹⁰ and R¹¹ are independently selected at each occurrence from hydrogen and C₁₋₃ alkyl.

In some embodiments, for a compound or modified protein of the present disclosure, R¹ is selected from C₆₋₁₀ aryl and 5- to 10-membered heteroaryl, each of which is optionally substituted with one, two, three, four, or five R²⁰. In some embodiments, R¹ is selected from naphthyl, isoquinolinyl, indazolyl, benzothiazolyl, benzothiophenyl, phenyl, and pyridinyl, each of which is optionally substituted with one or more R²⁰. In some embodiments, R¹ is substituted with one, two, three, or four substituents independently selected from halogen, —CN, C₁₋₃ alkyl, C₁₋₃ haloalkyl, C₂₋₃ alkenyl, C₂₋₃ alkynyl, —OR²², —N(R²²)(R²³), and C₃₋₆ cycloalkyl. In some embodiments, R¹ is substituted with one, two, three, or four substituents independently selected from halogen, —CN, —CH₃, —CH₂CH₃, —CH═CH₂, —CF₃, —C≡C, —OH, —NH₂, and -cyclopropyl. In some embodiments, R¹ is selected from

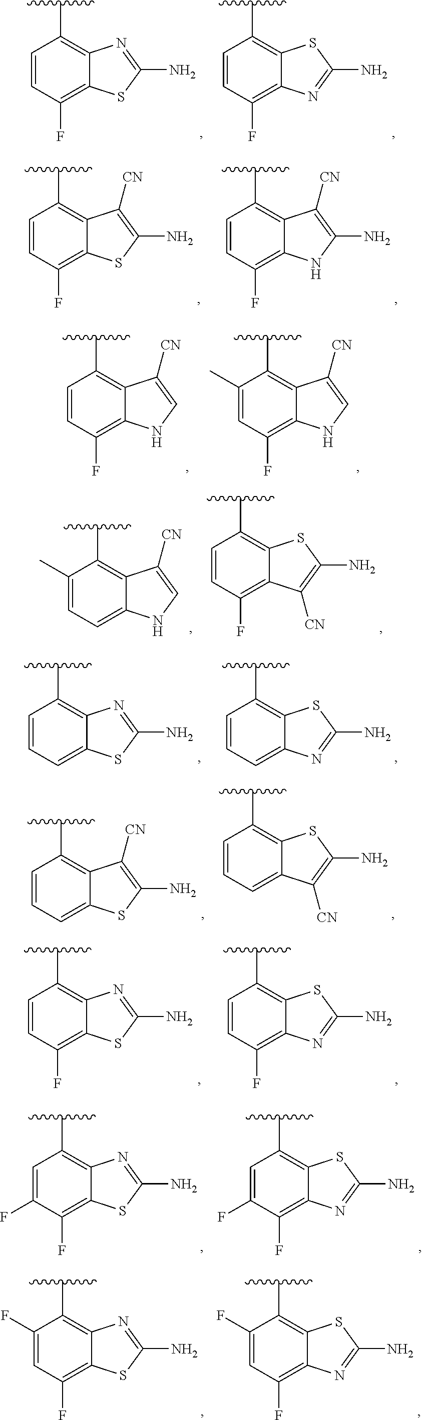

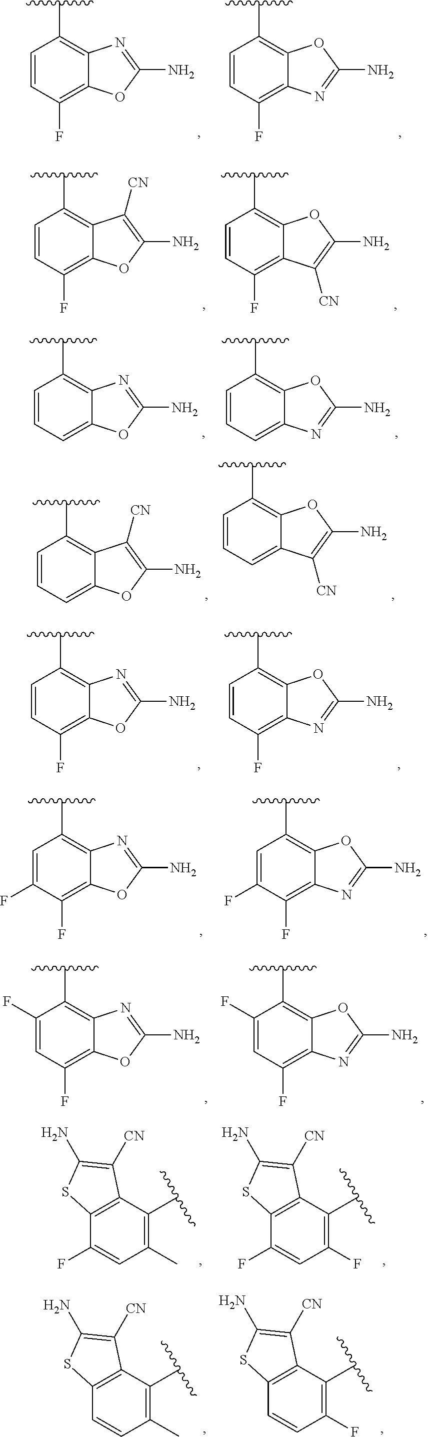

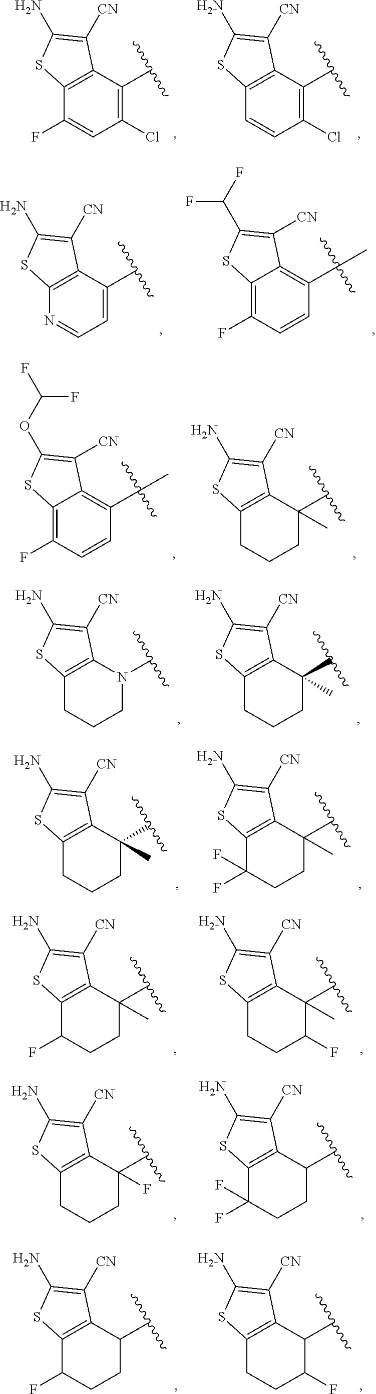

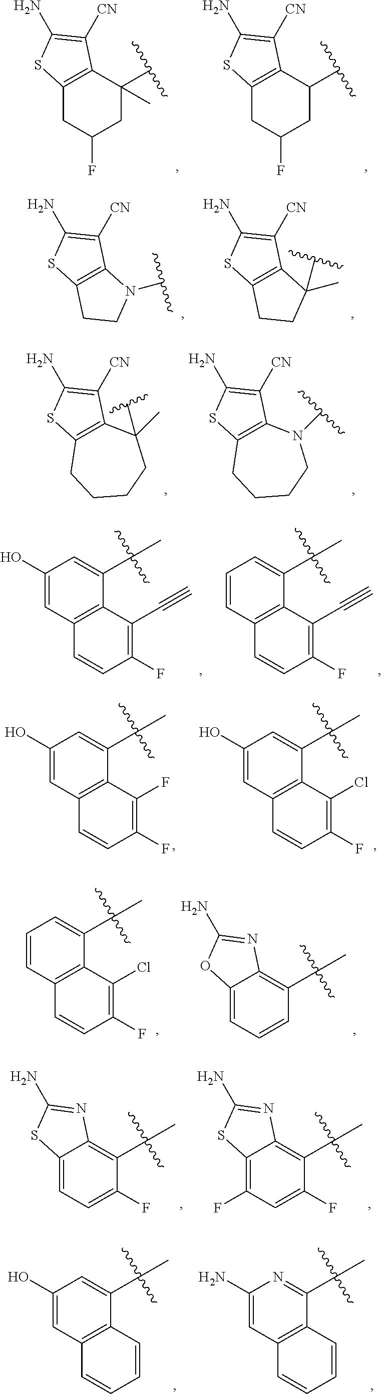

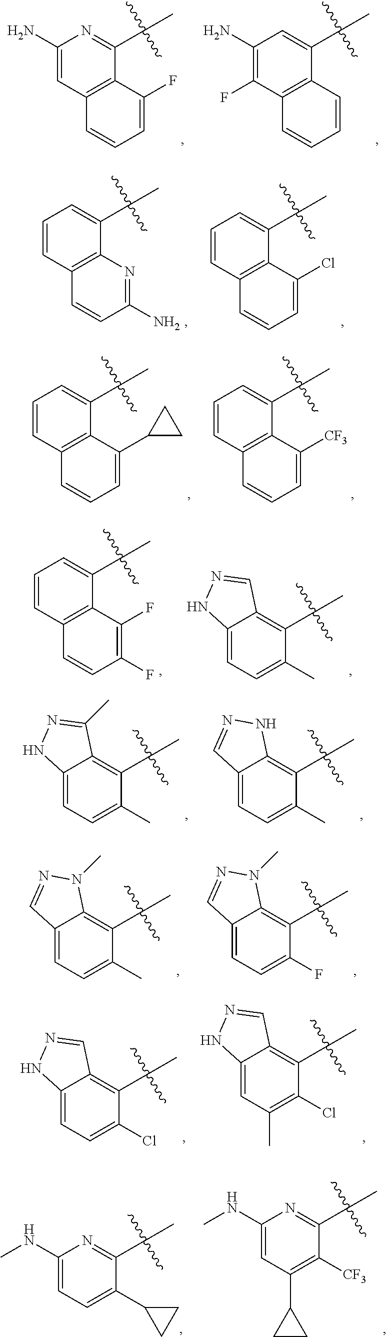

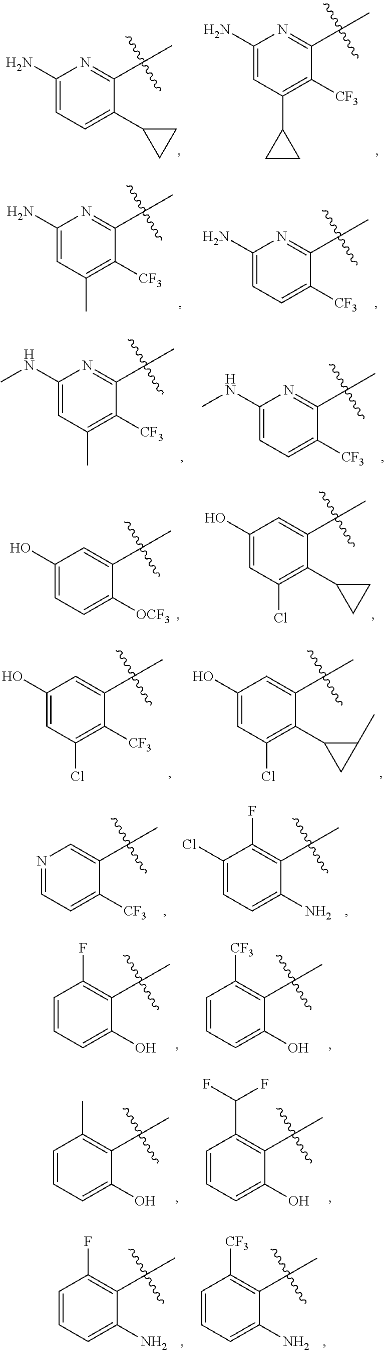

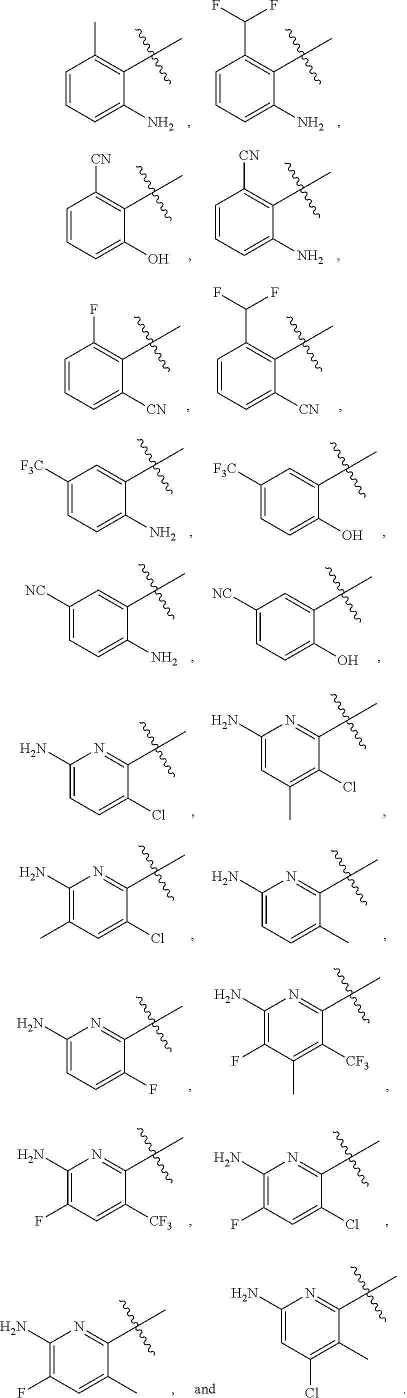

In some embodiments, R¹ is selected from

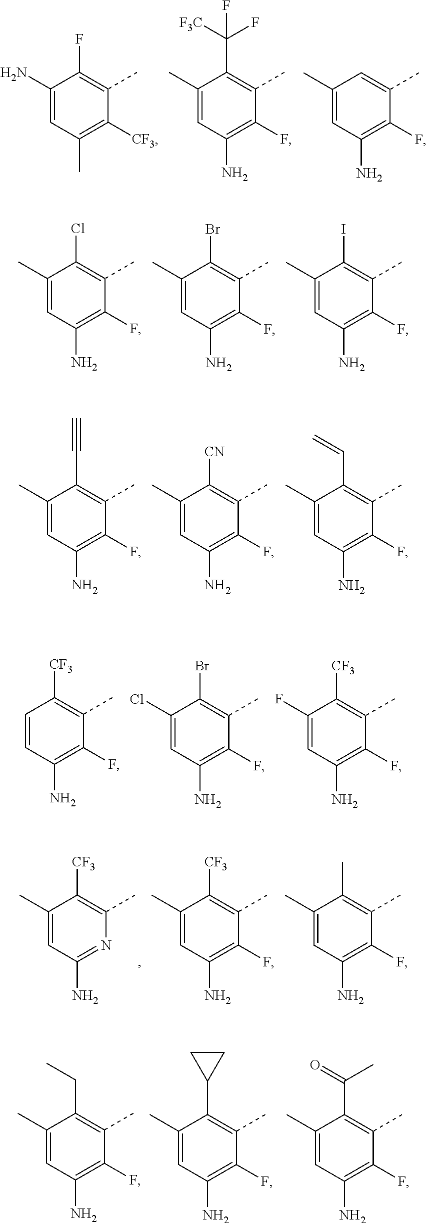

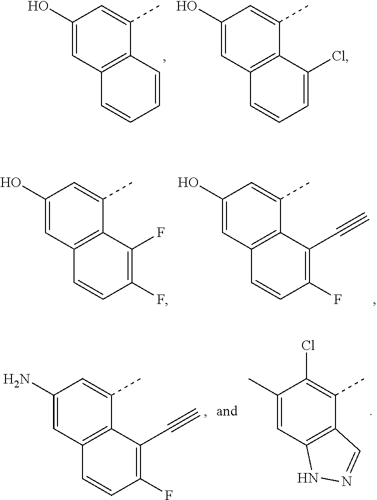

In some embodiments, R¹ is

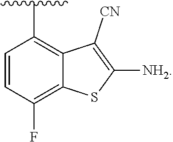 [SMILES: *c1ccc(F)c2sc(N)c(C#N)c12]

In some embodiments, for a compound or modified protein of the present disclosure, L¹ is 6- to 12-membered heterocycle optionally substituted with one or more R²⁰. In some embodiments, L¹ is 10-membered bicyclic heterocycle substituted with one, two, three, or four R²⁰. In some embodiments, L¹ comprises 1 to 5 nitrogen atoms. In some embodiments, L¹ is:

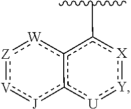

wherein:
W is N, C(R¹⁷), N(R^{17b}), C(R¹⁷)₂, C(O), S(O), or S(O);
Z is N, C(R¹⁷), N(R^{17b}), C(R¹⁷)₂, C(O), S(O), or S(O)₂; wherein W and Z are not both selected from C(O), S(O), and S(O)₂;
V and J are each independently selected from N, C(R¹), C(R¹⁷), N(R¹), N(R^{17b}), C(R¹)(R¹⁷), and C(R¹⁷)₂; wherein exactly one of V and J is C(R¹), N(R¹), or C(R¹)(R¹⁷);
U is N, C(R¹⁷), N(R^{17b}), C(R¹⁷)₂, S(O), S(O)₂, or C(O);
Y is N, C(R¹⁸), N(R^{17b}), C(R¹⁸)(R¹⁷), S(O), S(O)₂, or C(O);
X is N, C(R¹⁷), N(R^{17b}), or C(R¹⁷)₂;
R¹⁷ is independently selected at each occurrence from hydrogen, halogen, —CN, C₁₋₆ alkyl, C₂₋₆ alkenyl, C₂₋₆ alkynyl, 2- to 6-membered heteroalkyl, 2- to 6-membered heteroalkenyl, 2- to 6-membered heteroalkynyl, —C₀₋₆ alkyl-(C₃₋₁₂ carbocycle), -(2- to 6-membered heteroalkyl)-(C₃₋₁₂ carbocycle), —C₀₋₆ alkyl-(3- to 12-membered heterocycle), -(2- to 6-membered heteroalkyl)-(3- to 12-membered heterocycle), —OR¹², —SR¹², —N(R¹²)(R¹³), ═NR¹², —C(R¹⁴)₂, —C(O)OR¹², —OC(O)N(R¹²)(R¹³), —N(R¹²)C(O)N(R¹²)(R¹³), —N(R¹²)C(O)OR¹², —N(R¹²)S(O)₂R¹², —C(O)R¹², —S(O)R¹², —OC(O)R¹², —C(O)N(R¹²)(R¹³), —C(O)C(O)N(R¹²)(R¹³), —N(R¹²)C(O)R¹², —S(O)₂R¹², —S(O)(NR¹²)R¹², —S(O)₂N(R¹²)(R¹³), —S(═O)(═NR¹²)N(R¹²)(R¹³), and —OCH₂C(O)OR¹², wherein C₁₋₆ alkyl, C₂₋₆ alkenyl, C₂₋₆ alkynyl, 2- to 6-membered heteroalkyl, 2- to 6-membered heteroalkenyl, 2- to 6-membered heteroalkynyl, —C₀₋₆ alkyl-(C₃₋₁₂ carbocycle), -(2- to 6-membered heteroalkyl)-(C₃₋₁₂ carbocycle), —C₀₋₆ alkyl-(3- to 12-membered heterocycle), and -(2- to 6-membered heteroalkyl)-(3- to 12-membered heterocycle) are optionally substituted with one, two, or three R²⁰;

R^{17b} is independently selected at each occurrence from hydrogen, —CN, C₁₋₆ alkyl, C₂₋₆ alkenyl, C₂₋₆ alkynyl, —C₀₋₆ alkyl-(C₃₋₁₂ carbocycle), —C₀₋₆ alkyl-(3- to 12-membered heterocycle), —OR¹², —SR¹², —C(O)OR¹², —OC(O)N(R¹²)(R¹³), —C(O)R¹², —S(O)R¹², —OC(O)R¹², —C(O)N(R¹²)(R¹³), —C(O)C(O)N(R¹²)(R¹³), —S(O)₂R¹², —S(O)(NR¹²)R¹², —S(O)₂N(R¹²)(R¹³), and —S(═O)(═NR¹²)N(R¹²)(R¹³), wherein C₁₋₆ alkyl, C₂₋₆ alkenyl, C₂₋₆ alkynyl, —C₀₋₆ alkyl-(C₃₋₁₂ carbocycle), and —C₀₋₆ alkyl-(3- to 12-membered heterocycle) are optionally substituted with one, two, or three R²⁰;
R¹⁸ is selected from halogen, —CN, C₁₋₆ alkyl, C₂₋₆ alkenyl, C₂₋₆ alkynyl, 2- to 6-membered heteroalkyl, 2- to 6-membered heteroalkenyl, 2- to 6-membered heteroalkynyl, —C₀₋₆ alkyl-(C₃₋₁₂ carbocycle), -(2- to 6-membered heteroalkyl)-(C₃₋₁₂ carbocycle), —C₀₋₆ alkyl-(3- to 12-membered heterocycle), -(2- to 6-membered heteroalkyl)-(3- to 12-membered heterocycle), —OR¹², —SR¹², —N(R¹²)(R¹³), ═NR¹², ═C(R¹⁴)₂, —C(O)OR¹², —OC(O)N(R¹²)(R¹³), —N(R¹²)C(O)N(R¹²)(R¹³), —N(R¹²)C(O)OR¹², —N(R¹²)S(O)₂R¹², —C(O)R¹², —S(O)R¹², —OC(O)R¹², —C(O)N(R¹²)(R¹³), —C(O)C(O)N(R¹²)(R¹³), —N(R¹²)C(O)R¹², —S(O)₂R¹², —S(O)(NR¹²)R¹², —S(O)₂N(R¹²)(R¹³), —S(═O)(═NR¹²)N(R¹²)(R¹³), and —OCH₂C(O)OR¹², wherein C₁₋₆ alkyl, C₂₋₆ alkenyl, C₂₋₆ alkynyl, 2- to 6-membered heteroalkyl, 2- to 6-membered heteroalkenyl, 2- to 6-membered heteroalkynyl, —C₀₋₆ alkyl-(C₃₋₁₂ carbocycle), -(2- to 6-membered heteroalkyl)-(C₃₋₁₂ carbocycle), —C₀₋₆ alkyl-(3- to 12-membered heterocycle), and -(2- to 6-membered heteroalkyl)-(3- to 12-membered heterocycle) are optionally substituted with one, two, or three R²⁰;
R¹² is independently selected at each occurrence from hydrogen, C₁₋₆ alkyl, C₂₋₆ alkenyl, C₂₋₆ alkynyl, —C₀₋₆ alkyl-(C₃₋₁₂ carbocycle), and —C₀₋₆ alkyl-(3- to 12-membered heterocycle), wherein C₁₋₆ alkyl, C₂₋₆ alkenyl, C₂₋₆ alkynyl, —C₀₋₆ alkyl-(C₃₋₁₂ carbocycle), and —C₀₋₆ alkyl-(3- to 12-membered heterocycle) are optionally substituted with one, two, or three R²⁰;
R¹³ is independently selected at each occurrence from hydrogen, C₁₋₆ alkyl, and C₁₋₆ haloalkyl; or R¹² and R₁₃ attached to the same nitrogen atom form 3- to 10-membered heterocycle optionally substituted with one, two, or three R²⁰;
R¹⁴ is independently selected at each occurrence from hydrogen, halogen, C₁₋₆ alkyl, C₂₋₆ alkenyl, C₂₋₆ alkynyl, —C₀₋₆ alkyl-(C₃₋₁₂ carbocycle), and —C₀₋₆ alkyl-(3- to 12-membered heterocycle), or two R¹⁴ are taken together with the carbon atom to which they are attached to form C₃₋₁₂ carbocycle or 3- to 12-membered heterocycle, wherein C₁₋₆ alkyl, C₂₋₆ alkenyl, C₂₋₆ alkynyl, —C₀₋₆ alkyl-(C₃₋₁₂ carbocycle), —C₀₋₆ alkyl-(3- to 12-membered heterocycle), C₃₋₁₂ carbocycle, and 3- to 12-membered heterocycle are optionally substituted with one, two, or three R²⁰; and
[symbol] indicates a single or double bond such that all valences are satisfied.

In some embodiments, for a compound or modified protein of the present disclosure, W is C(R¹⁷), C(R¹⁷)₂, or C(O); Z is N, C(R¹⁷), N(R^{17b}), or C(R¹⁷)₂; V is C(R¹) or N(R¹); and J is C(R¹⁷) or C(R¹⁷)₂. In some embodiments, W is CH, CH₂, or C(O); Z is N, CCI, N(R^{17b}), or CH₂; V is C(R¹) or N(R¹); and J is CF or CH₂. In some embodiments, W is C(R¹⁷); Z is C(R¹⁷); V is C(R¹); and J is C(R¹⁷). In some embodiments, W is CH; Z is CCl; V is C(R¹); and J is CF. In some embodiments, U is N; Y is C(R¹⁸); and X is N. In some embodiments, R¹⁸ is selected from hydrogen, C₁₋₃ alkyl, —OR¹², and 3- to 10-membered heterocycle, wherein C₁₋₃ alkyl and 3- to 10-membered heterocycle are optionally substituted with one, two, or three R²⁰. In some embodiments, R¹⁸ is-OR¹². In some embodiments, R¹⁸ is-O(C₁₋₃ alkylene) (4- to 10-membered heterocycle), wherein 4- to 10-membered heterocycle is optionally substituted with one, two, or three substituents independently selected from halogen, C₁₋₃ alkyl, C₁₋₃ haloalkyl, and ═C(R²¹)₂, wherein R²¹ is independently selected at each occurrence from hydrogen, halogen, and C₁₋₃ alkyl. In some embodiments, R¹⁸ is selected from

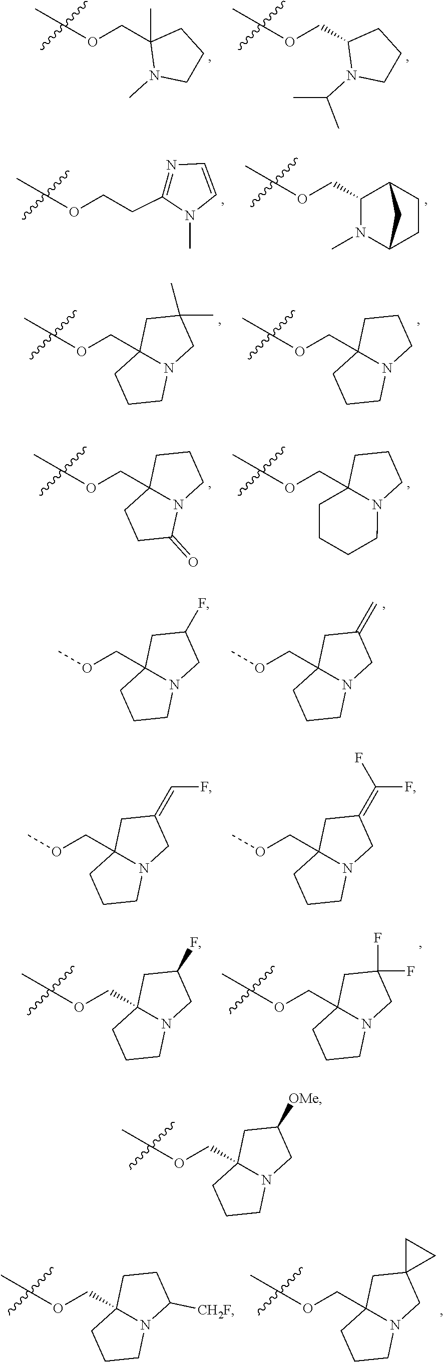

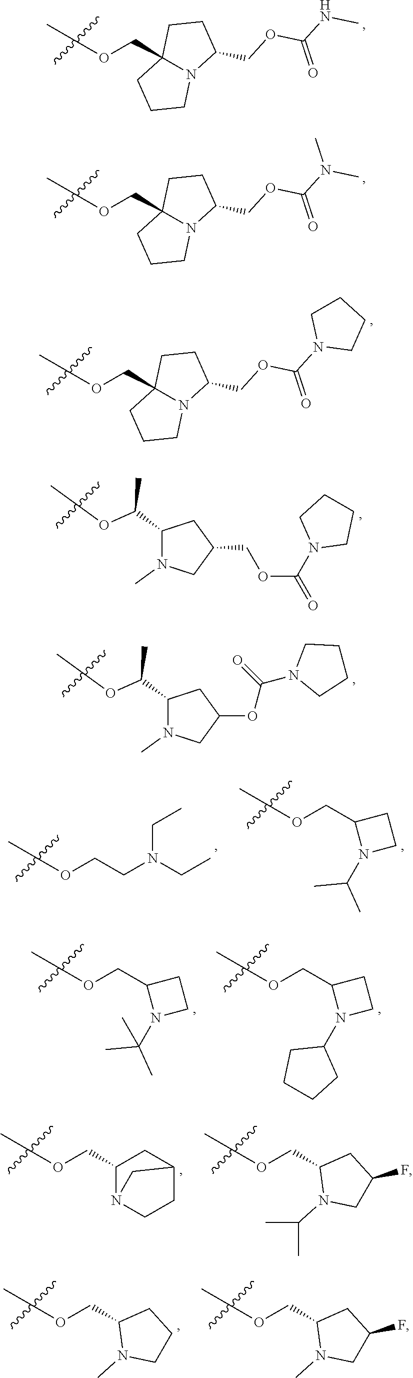

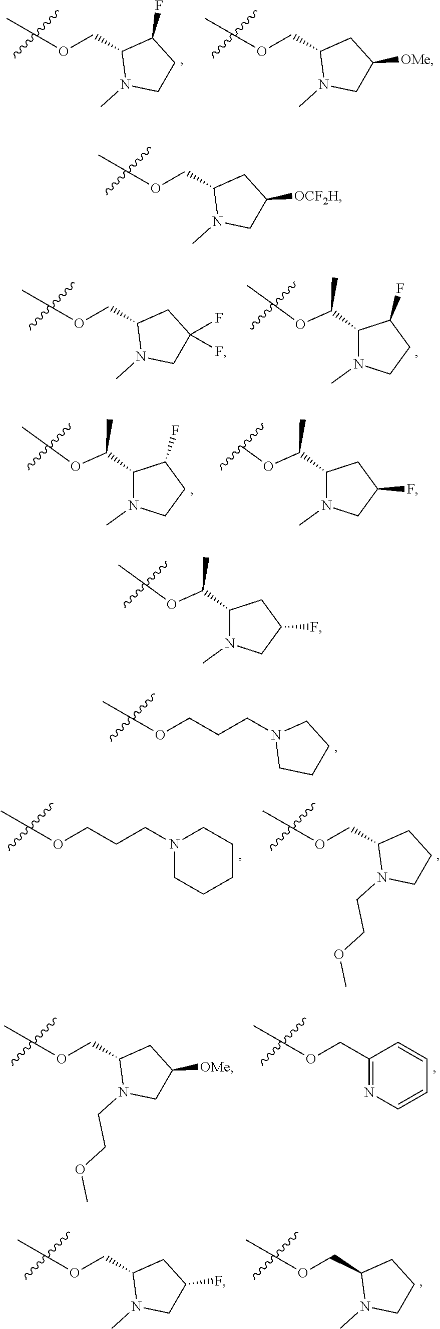

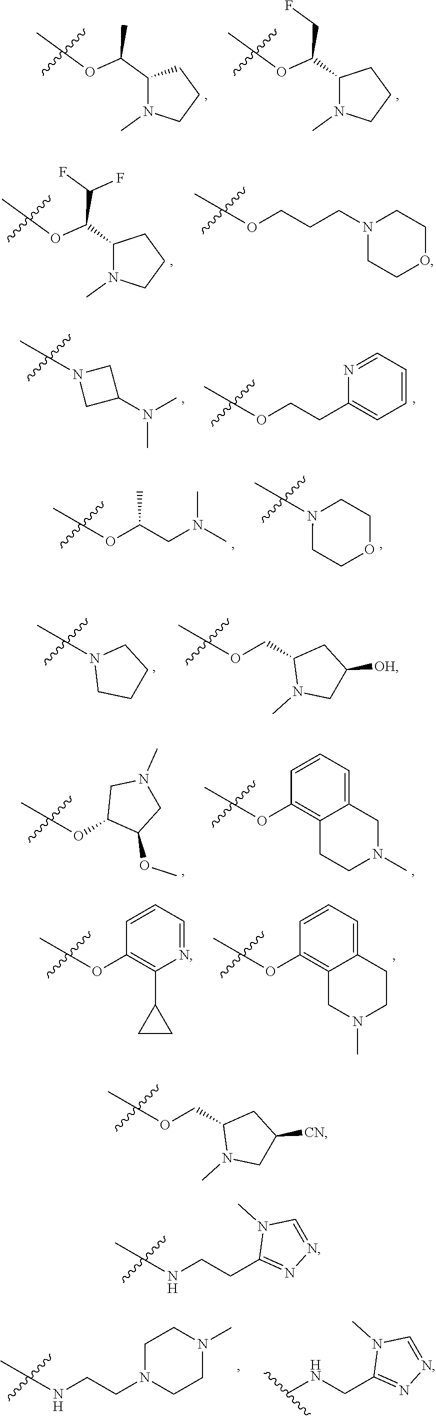

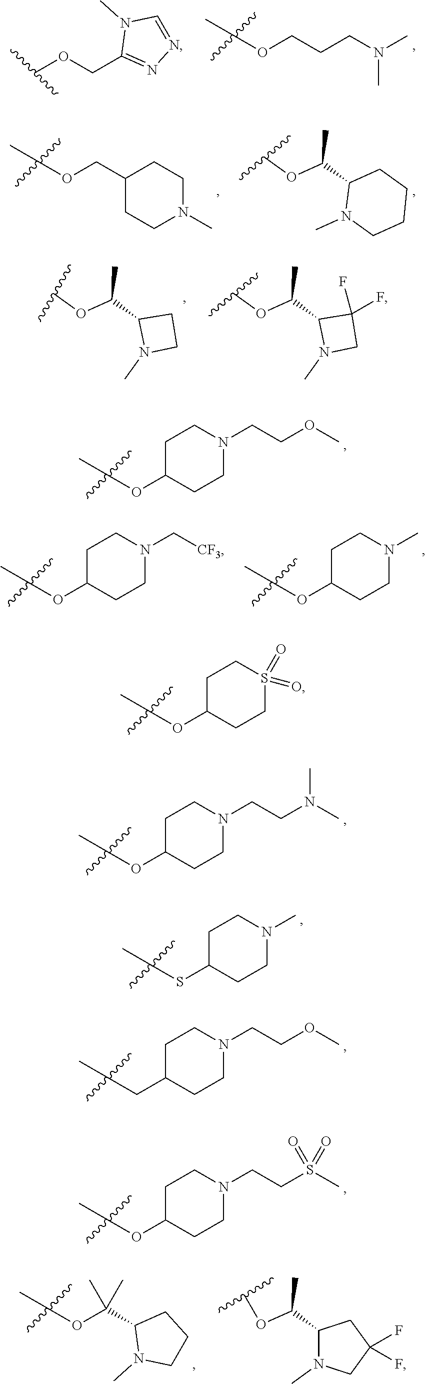

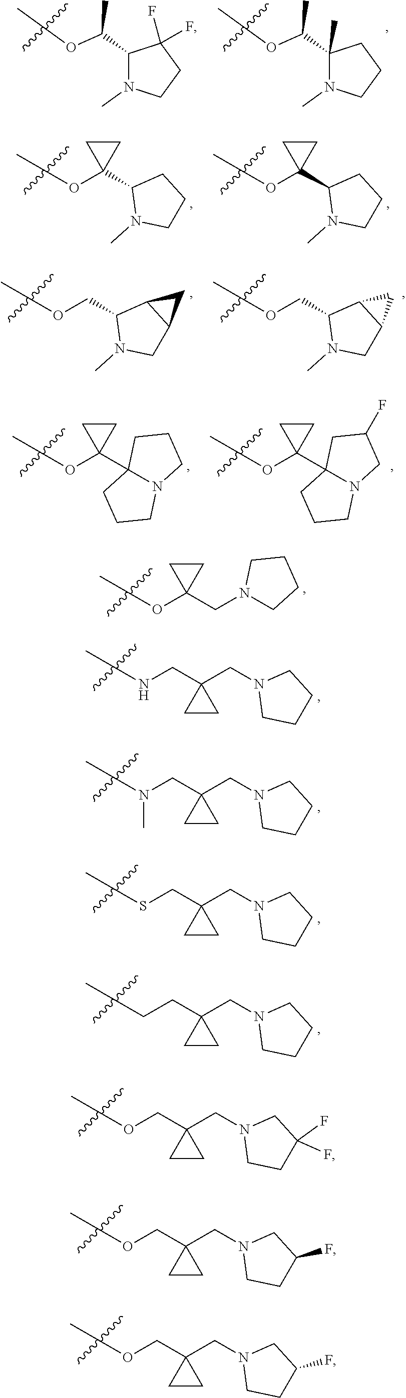

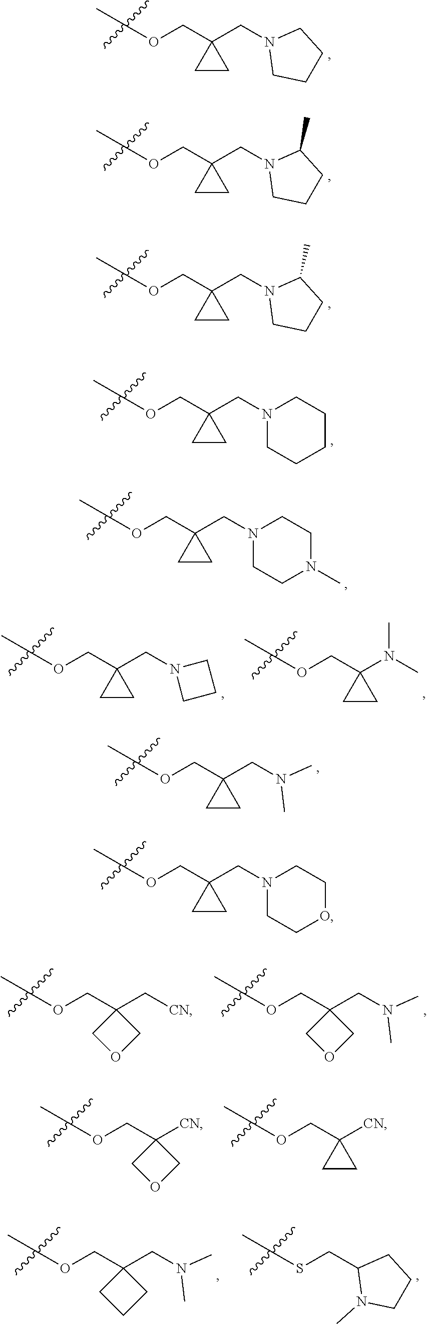

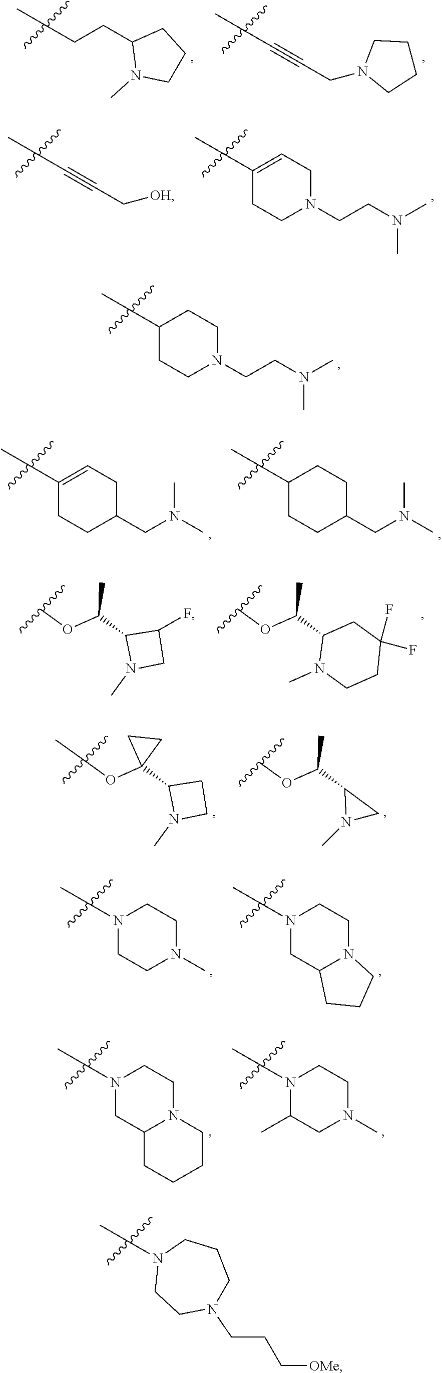

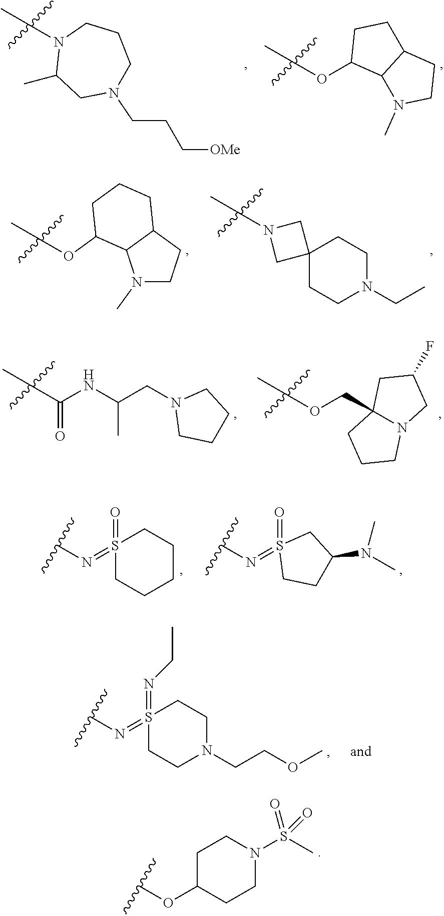

In some embodiments, R¹⁸ is selected from

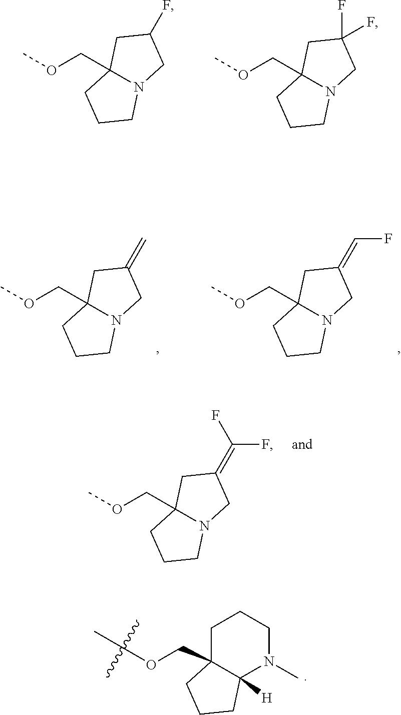

In some embodiments, for a compound or modified protein of the present disclosure, L¹ is

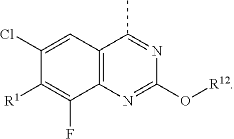

In some embodiments, R¹-L¹ is

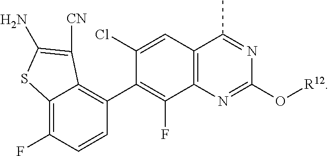

In some embodiments, R¹-L¹ is

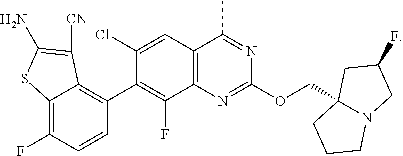

In some embodiments, L² is selected from a bond, C₁₋₃ alkylene, and 2- to 3-membered heteroalkylene, wherein C₁₋₃ alkylene and 2- to 3-membered heteroalkylene are optionally substituted with one, two, or three R²⁰. In some embodiments, L² is selected from a bond, C₁₋₃ alkylene, —N(H)C₁₋₃ alkylene-, —N(C₁₋₃ alkyl)C₁₋₃ alkylene-, and —N(C₃₋₆ cycloalkyl) C₁₋₃ alkylene-, wherein C₁₋₃ alkylene, C₁₋₃ alkyl, and C₃₋₆cycloalkyl are optionally substituted with one, two, or three substituents selected from halogen, C₁₋₃ alkyl, and C₁₋₃ haloalkyl. In some embodiments, L² is a bond.

In some embodiments, for a compound or modified protein of the present disclosure, R² is selected from C₁₋₆ alkyl and C₃₋₆ cycloalkyl. In some embodiments, R³ is selected from hydrogen and C₁₋₆ alkyl, such as R³ is hydrogen. In some embodiments, R⁴, R⁵, and R⁶ are independently selected from hydrogen, C₁₋₃ alkyl, and —(C₁₋₃ alkyl)CN, or R⁴ and R⁵, together with the carbon atom to which they are attached, form C₃₋₆ cycloalkyl. In some embodiments, R⁷ is

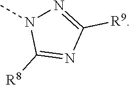

In some embodiments, R⁸ is selected from hydrogen, halogen, —CH₃, —CH₂F, —CHF₂, and —CF₃, such as R⁸ is hydrogen. In some embodiments, R⁹ is selected from hydrogen, halogen, —CH₃, —CH₂F, —CHF₂, and —CF₃, such as R⁹ is selected from hydrogen and chloro.

In some embodiments, for a compound described herein, R¹ is selected from naphthyl, isoquinolinyl, indazolyl, benzothiazolyl, benzothiophenyl, phenyl, and pyridinyl, each of which is optionally substituted with one or more R²⁰; L¹ is 10-membered bicyclic heterocycle substituted with one, two, three, or four R²⁰; L² is selected from a bond, C₁₋₃ alkylene, and 2- to 3-membered heteroalkylene, wherein C₁₋₃ alkylene and 2- to 3-membered heteroalkylene are optionally substituted with one, two, or three R²⁰; and R⁷ is

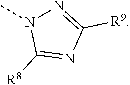

In some embodiments, R¹-L¹ is

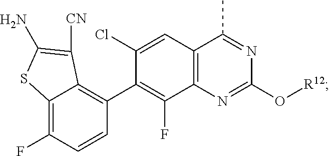

L² is selected from a bond, C₁₋₃ alkylene, and 2- to 3-membered heteroalkylene, wherein C₁₋₃ alkylene and 2- to 3-membered heteroalkylene are optionally substituted with one, two, or three R²⁰; and R⁷ is

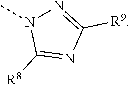

In some embodiments, a compound described herein reversibly binds to a K-Ras protein when R⁷ is replaced with hydrogen. In some embodiments, the compound reversibly binds to a K-Ras protein with an IC₅₀ of less than 1000 nM, less than 250 nM, less than 100 nM, or even less, as assessed by an HTRF assay when R⁷ is replaced with hydrogen. In some embodiments, a compound described herein reversibly binds to a K-Ras protein when —C(O)R⁷ is replaced with hydrogen. In some embodiments, the compound reversibly binds to a K-Ras protein with an IC₅₀ of less than 1000 nM, less than 250 nM, less than 100 nM, or even less, as assessed by an HTRF assay when —C(O)R⁷ is replaced with hydrogen.

In some embodiments, the present disclosure provides a compound disclosed herein, or a pharmaceutically acceptable salt or solvate thereof. In some embodiments, the present disclosure provides a pharmaceutical composition comprising a compound described herein, or a pharmaceutically acceptable salt or solvate thereof, and a pharmaceutically acceptable excipient.

In certain aspects, the present disclosure provides a method of modifying a Ras mutant protein, comprising contacting the Ras mutant protein with an effective amount of a compound, salt, or solvate described herein. In some embodiments, the modified Ras mutant protein exhibits a reduced Ras signaling output. The reduced Ras signaling output may be evidenced by one or more output selected from (i) an increase in steady state level of GDP-bound modified protein; (ii) a reduction in steady state level of GTP-bound modified protein; (iii) a reduction of phosphorylated AKTs473; (iv) a reduction of phosphorylated ERK T202/Y204; (v) a reduction of phosphorylated S6 S235/236; (vi) a reduction of cell growth of a tumor cell expressing a Ras G12S mutant protein; and (vii) a reduction in Ras interaction with a Ras-pathway signaling protein. In some embodiments, the Ras mutant protein comprises an amino acid sequence selected from SEQ ID No. 1, SEQ ID No. 3, SEQ ID No. 5, and a respective fragment thereof comprising a serine residue corresponding to position 12 of SEQ ID No. 1. In some embodiments, the Ras mutant protein comprises an amino acid sequence of SEQ ID No. 1, or a fragment thereof comprising the serine residue corresponding to position 12 of SEQ ID No. 1. In some embodiments, the contacting results in release of a leaving group, such as a leaving group selected from

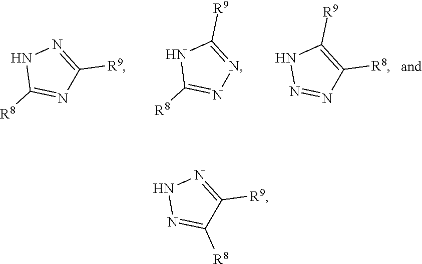

or a salt or tautomer thereof. In some embodiments, the modified Ras mutant protein comprises an amino acid sequence of SEQ ID No. 1, or a fragment thereof that comprises the serine residue corresponding to position 12 of SEQ ID No. 1, and wherein the compound selectively labels the serine residue as compared to (i) an aspartate residue of a K-Ras G12D mutant protein, said aspartate corresponding to position 12 of SEQ ID No. 2; (ii) a valine residue of a K-Ras G12V mutant protein, said valine corresponding to position 12 of SEQ ID No. 3; and/or (iii) a glycine residue of a K-Ras wildtype protein, said glycine corresponding to position 12 of SEQ ID No. 1. In some embodiments, the compound selectively labels the serine residue by at least 2-fold, 3-fold, 4-fold, 5-fold, or more when assayed under comparable conditions. In some embodiments, the contacting occurs in vivo or in vitro.

In certain aspects, the present disclosure provides a method of treating cancer in a subject in need thereof, comprising administering to the subject a therapeutically effective amount of a compound described herein, or a pharmaceutically acceptable salt or solvate thereof. In some embodiments, the present disclosure provides a method of treating cancer in a subject comprising a Ras mutant protein, the method comprising: modifying the Ras mutant protein of said subject by administering to said subject a compound described herein, or a pharmaceutically acceptable salt or solvate thereof, wherein the compound is characterized in that upon contacting the Ras mutant protein, said Ras mutant protein is modified covalently at a residue corresponding to reside 12 of SEQ ID No: 1, such that said modified Ras mutant protein exhibits reduced Ras signaling output. In some embodiments, the cancer is a solid tumor or a hematological cancer. In some embodiments, the cancer comprises a K-Ras G12S mutant protein.

In certain aspects, the present disclosure provides a method of modulating signaling output of a Ras protein, comprising contacting a Ras protein with an effective amount of a compound described herein, or a pharmaceutically acceptable salt or solvate thereof, thereby modulating the signaling output of the Ras protein. In certain aspects, the present disclosure provides a method of inhibiting cell growth, comprising administering an effective amount of a compound described herein, or a pharmaceutically acceptable salt or solvate thereof, to a cell expressing a Ras protein, thereby inhibiting growth of said cells.

Any of the methods described herein may further comprise administering an additional agent. In some embodiments, the additional agent comprises (1) an inhibitor of MEK; (2) an inhibitor of epidermal growth factor receptor (EGFR) and/or mutants thereof; (3) an immunotherapeutic agent; (4) a taxane; (5) an anti-metabolite; (6) an inhibitor of FGFR1 and/or FGFR2 and/or FGFR3 and/or mutants thereof; (7) a mitotic kinase inhibitor; (8) an anti-angiogenic drug; (9) a topoisomerase inhibitor; (10) a platinum-containing compound; (11) an inhibitor of c-MET and/or mutants thereof; (12) an inhibitor of BCR-ABL and/or mutants thereof; (13) an inhibitor of ErbB2 (Her2) and/or mutants thereof; (14) an inhibitor of AXL and/or mutants thereof; (15) an inhibitor of NTRK1 and/or mutants thereof; (16) an inhibitor of RET and/or mutants thereof; (17) an inhibitor of A-Raf and/or B-Raf and/or C-Raf and/or mutants thereof; (18) an inhibitor of ERK and/or mutants thereof; (19) an MDM2 inhibitor; (20) an inhibitor of mTOR; (21) an inhibitor of IGF1/2 and/or IGF1-R; (22) an inhibitor of CDK9; (23) an inhibitor of farnesyl transferase; (24) an inhibitor of SHIP pathway; (25) an inhibitor of SRC; (26) an inhibitor of JAK; (27) a PARP inhibitor, (28) a ROS1 inhibitor; (29) an inhibitor of SHP pathway; (30) an inhibitor of Src, FLT3, HDAC, VEGFR, PDGFR, LCK, Bcr-Abl or AKT; (31) an inhibitor of KrasG12C; (32) an SHC inhibitor (e.g., PP2, AID371185); (33) a GAB inhibitor; (34) a PI-3 kinase inhibitor; (35) a MARPK inhibitor; (36) a CDK4/6 inhibitor; (37) a MAPK inhibitor; (38) a SHP2 inhibitor; (39) a checkpoint immune blockade agent; (40) a SOS1 inhibitor; or (41) a SOS2 inhibitor. In some embodiments, the additional agent comprises an inhibitor of SHP2 selected RMC-4630, ERAS-601, TNO155, JAB-3068, IACS-13909/BBP-398, SHP099, and RMC-4550. In some embodiments, the additional agent comprises an inhibitor of SOS selected from BI-3406, MRTX0902, BAY 293, RMC-5845, and BI-1701963. In some embodiments, the additional agent comprises an inhibitor of EGFR selected from afatinib, erlotinib, gefitinib, lapatinib, cetuximab panitumumab, osimertinib, olmutinib, and EGF-816. In some embodiments, the additional agent comprises an inhibitor of MEK selected from trametinib, cobimetinib, binimetinib, selumetinib, refametinib, and AZD6244. In some embodiments, the additional agent comprises an inhibitor of ERK selected from ulixertinib, MK-8353, LTT462, AZD0364, SCH772984, BIX02189, LY3214996, and ravoxertinib. In some embodiments, the additional agent comprises an inhibitor of CDK4/6 selected from palbociclib, ribociclib, and abemaciclib. In some embodiments, the additional agent comprises an inhibitor of BRAF selected from Sorafenib, Vemurafenib, Dabrafenib, Encorafenib, regorafenib, and GDC-879.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1 depicts a sequence alignment of various wild type Ras proteins including K-Ras, H-Ras, N-Ras, Ra1A, and Ra1B, from top to bottom.

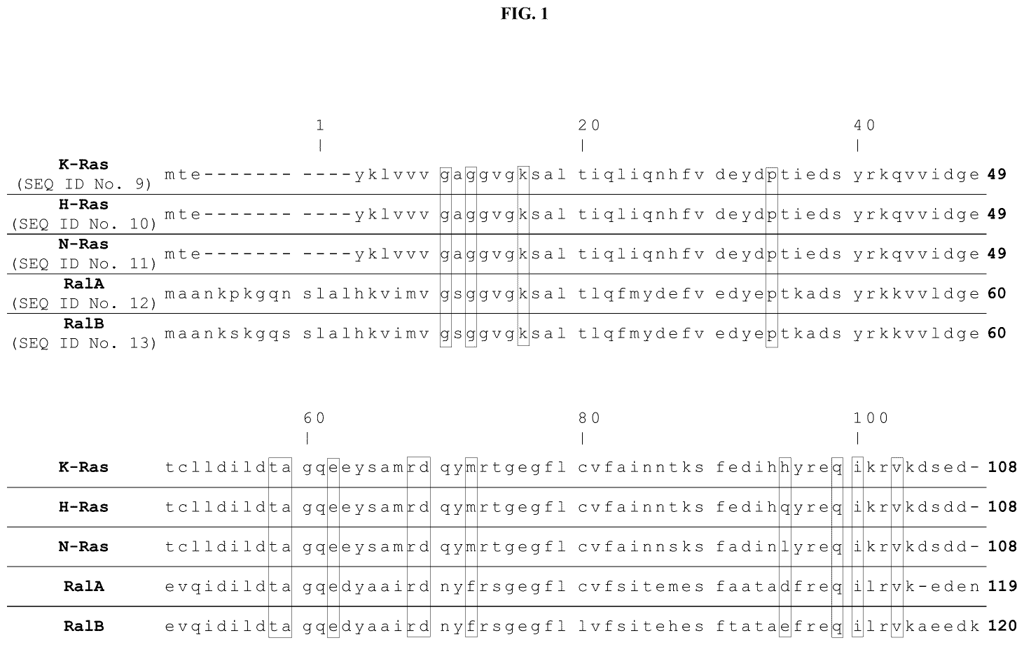

### DETAILED DESCRIPTION

Unless defined otherwise, all technical and scientific terms used herein have the same meaning as is commonly understood by one of skill in the art to which this disclosure belongs. In the event that there are a plurality of definitions for terms herein, those in this section prevail. All patents, patent applications, publications and published nucleotide and amino acid sequences (e.g., sequences available in GenBank or other databases) referred to herein are incorporated by reference. Chemical structures are named herein according to IUPAC conventions as implemented in ChemDraw® software (Perkin Elmer, Inc., Cambridge, MA). The section headings used herein are for organizational purposes only and are not to be construed as limiting the subject matter described. As used in the specification and claims, the singular forms “a”, “an” and “the” include plural references unless the context clearly dictates otherwise. Furthermore, use of the term “including” as well as other forms, such as “include”, “includes”, and “included”, is not limiting. The section headings used herein are for organizational purposes only and are not to be construed as limiting the subject matter described.

The term “C_{x-y}” or “Cₓ-C_{y}” when used in conjunction with a chemical moiety, such as alkyl, alkenyl, or alkynyl, is meant to include groups that contain from x to y carbons in the chain. For example, the term “C_{x-y} alkyl” refers to substituted or unsubstituted saturated hydrocarbon groups, including straight-chain alkyl and branched-chain alkyl groups, that contain from x to y carbons in the chain.

“Alkyl” refers to substituted or unsubstituted saturated hydrocarbon groups, including linear and branched alkyl groups. An alkyl group may contain from one to twelve carbon atoms (e.g., C₁₋₁₂ alkyl), such as one to eight carbon atoms (C₁₋₈ alkyl) or one to six carbon atoms (C₁₋₆ alkyl). Exemplary alkyl groups include methyl, ethyl, n-propyl, isopropyl, n-butyl, isobutyl, sec-butyl, tert-butyl, pentyl, isopentyl, neopentyl, hexyl, septyl, octyl, nonyl, and decyl. An alkyl group is attached to the rest of the molecule by a single bond. Unless stated otherwise specifically in the specification, an alkyl group is optionally substituted by one or more substituents such as those substituents described herein.

“Haloalkyl” refers to an alkyl group that is substituted by one or more halogens. Exemplary haloalkyl groups include trifluoromethyl, difluoromethyl, trichloromethyl, 2,2,2-trifluoroethyl, 1,2-difluoroethyl, 3-bromo-2-fluoropropyl, and 1,2-dibromoethyl.

“Alkenyl” refers to substituted or unsubstituted hydrocarbon groups, including linear and branched alkenyl groups, containing at least one double bond. An alkenyl group may contain from two to twelve carbon atoms (e.g., C₂₋₁₂ alkenyl), such as two to eight carbon atoms (C₂₋₈ alkenyl) or two to six carbon atoms (C₂₋₆ alkenyl). Exemplary alkenyl groups include ethenyl (i.e., vinyl), prop-1-enyl, but-1-enyl, pent-1-enyl, penta-1,4-dienyl, and the like. Unless stated otherwise specifically in the specification, an alkenyl group is optionally substituted by one or more substituents such as those substituents described herein.

“Alkynyl” refers to substituted or unsubstituted hydrocarbon groups, including linear and branched alkynyl groups, containing at least one triple bond. An alkynyl group may contain from two to twelve carbon atoms (e.g., C₂₋₁₂ alkynyl), such as two to eight carbon atoms (C₂₋₈ alkynyl) or two to six carbon atoms (C₂₋₆ alkynyl). Exemplary alkynyl groups include ethynyl, propynyl, butynyl, pentynyl, hexynyl, and the like. Unless stated otherwise specifically in the specification, an alkynyl group is optionally substituted by one or more substituents such as those substituents described herein.

“Alkylene” or “alkylene chain” refers to substituted or unsubstituted divalent saturated hydrocarbon groups, including linear alkylene and branched alkylene groups, that contain from one to twelve carbon atoms (e.g., C₁₋₁₂ alkylene), such as one to eight carbon atoms (C₁₋₈ alkylene) or one to six carbon atoms (C₁₋₆ alkylene). Exemplary alkylene groups include methylene, ethylene, propylene, and n-butylene. Similarly, “alkenylene” and “alkynylene” refer to alkylene groups, as defined above, which comprise one or more carbon-carbon double or triple bonds, respectively. The points of attachment of the alkylene, alkenylene or alkynylene chain to the rest of the molecule can be through one carbon or any two carbons of the chain. Unless stated otherwise specifically in the specification, an alkylene, alkenylene, or alkynylene group is optionally substituted by one or more substituents such as those substituents described herein.

“Heteroalkyl”, “heteroalkenyl” and “heteroalkynyl” refer to substituted or unsubstituted alkyl, alkenyl and alkynyl groups, respectively, in which one or more, such as 1, 2 or 3, of the carbon atoms are replaced with a heteroatom, such as O, N, P, Si, S, or combinations thereof. Any nitrogen, phosphorus, and sulfur heteroatoms present in the chain may optionally be oxidized, and any nitrogen heteroatoms may optionally be quaternized. If given, a numerical range refers to the chain length in total. For example, a 3- to 8-membered heteroalkyl group has a chain length of 3 to 8 atoms. Connection to the rest of the molecule may be through either a heteroatom or a carbon in the heteroalkyl, heteroalkenyl, or heteroalkynyl chain. Unless stated otherwise specifically in the specification, a heteroalkyl, heteroalkenyl, or heteroalkynyl group is optionally substituted by one or more substituents such as those substituents described herein.

“Heteroalkylene”, “heteroalkenylene” and “heteroalkynylene” refer to substituted or unsubstituted alkylene, alkenylene and alkynylene groups, respectively, in which one or more, such as 1, 2 or 3, of the carbon atoms are replaced with a heteroatom, such as O, N, P, Si, S, or combinations thereof. Any nitrogen, phosphorus, and sulfur heteroatoms present in the chain may optionally be oxidized, and any nitrogen heteroatoms may optionally be quaternized. If given, a numerical range refers to the chain length in total. For example, a 3- to 8-membered heteroalkylene group has a chain length of 3 to 8 atoms. The points of attachment of the heteroalkylene, heteroalkenylene or heteroalkynylene chain to the rest of the molecule can be through either one heteroatom or one carbon, or any two heteroatoms, any two carbons, or any one heteroatom and any one carbon in the heteroalkylene, heteroalkenylene or heteroalkynylene chain. Unless stated otherwise specifically in the specification, a heteroalkylene, heteroalkenylene, or heteroalkynylene group is optionally substituted by one or more substituents such as those substituents described herein.

“Carbocycle” refers to a saturated, unsaturated or aromatic ring in which each atom of the ring is a carbon atom. Carbocycle may include C₃₋₁₀ monocyclic rings, C₆₋₁₂ bicyclic rings, C₇₋₁₈ polycyclic rings, C₅₋₁₂ spirocyclic rings, and C₆₋₁₂ bridged rings. Each ring of a bicyclic or polycyclic carbocycle may be selected from saturated, unsaturated, and aromatic rings. In some embodiments, the carbocycle is a C₆₋₁₂ aryl group, such as C₆₋₁₀ aryl. In some embodiments, the carbocycle is a C₃₋₁₂ cycloalkyl group. In some embodiments, the carbocycle is a C₅₋₁₂ cycloalkenyl group. In an exemplary embodiment, an aromatic ring, e.g., phenyl, may be fused to a saturated or unsaturated ring, e.g., cyclohexane, cyclopentane, or cyclohexene. Any combination of saturated, unsaturated and aromatic bicyclic rings, as valence permits, are included in the definition of carbocycle. A carbocycle may comprise a fused ring, a bridged ring, a spirocyclic ring, a saturated ring, an unsaturated ring, an aromatic ring, or any combination thereof. Exemplary carbocycles include cyclopentyl, cyclohexyl, cyclohexenyl, adamantly, phenyl, indanyl, and naphthyl. Unless state otherwise specifically in the specification, a carbocycle is optionally substituted by one or more substituents such as those substituents described herein.

“Heterocycle” refers to a saturated, unsaturated or aromatic ring comprising one or more heteroatoms, for example 1, 2 or 3 heteroatoms selected from O, S and N. Heterocycle may include 3- to 10-membered monocyclic rings, 6- to 12-membered bicyclic rings, 7- to 18-membered polycyclic rings, 5- to 12-membered spirocyclic rings, and 6- to 12-membered bridged rings. Each ring of a bicyclic or polycyclic heterocycle may be selected from saturated, unsaturated, and aromatic rings. The heterocycle may be attached to the rest of the molecule through any atom of the heterocycle, valence permitting, such as a carbon or nitrogen atom of the heterocycle. In some embodiments, the heterocycle is a 5- to 10-membered heteroaryl group, such as 5- or 6-membered heteroaryl. In some embodiments, the heterocycle is a 3- to 12-membered heterocycloalkyl group. A heterocycle may comprise a fused ring, a bridged ring, a spirocyclic ring, a saturated ring, an unsaturated ring, an aromatic ring, or any combination thereof. In an exemplary embodiment, a heterocycle, e.g., pyridyl, may be fused to a saturated or unsaturated ring, e.g., cyclohexane, cyclopentane, or cyclohexene. Exemplary heterocycles include pyrrolidinyl, pyrrolyl, imidazolyl, pyrazolyl, triazolyl, piperidinyl, pyridinyl, pyrimidinyl, pyridazinyl, pyrazinyl, thiophenyl, oxazolyl, thiazolyl, morpholinyl, indazolyl, indolyl, and quinolinyl. Unless stated otherwise specifically in the specification, a heterocycle is optionally substituted by one or more substituents such as those substituents described herein.

“Heteroaryl” refers to an aromatic ring that comprises at least one heteroatom, for example 1, 2 or 3 heteroatoms selected from O, S and N. Heteroaryl may include 5- to 10-membered monocyclic rings, 6- to 12-membered bicyclic rings, 7- to 18-membered polycyclic rings, 5- to 12-membered spirocyclic rings, and 6- to 12-membered bridged rings. As used herein, the heteroaryl ring may be selected from monocyclic or bicyclic—including fused, spirocyclic and bridged ring systems—wherein at least one of the rings in the ring system is aromatic. The heteroatom(s) in the heteroaryl may optionally be oxidized. One or more nitrogen atoms, if present, are optionally quaternized. The heteroaryl may be attached to the rest of the molecule through any atom of the heteroaryl, valence permitting, such as a carbon or nitrogen atom of the heteroaryl. Examples of heteroaryl groups include, but are not limited to, azepinyl, benzimidazolyl, benzisothiazolyl, benzisoxazolyl, benzofuranyl, benzothiazolyl, benzothiophenyl, benzoxazolyl, furanyl, imidazolyl, indazolyl, indolyl, isoquinolinyl, isothiazolyl, isoxazolyl, oxadiazolyl, oxazolyl, purinyl, pyrazinyl, pyrazolidinyl, pyrazolyl, pyridazinyl, pyridazolyl, pyridyl, pyrimidinyl, pyrrolyl, quinazolinyl, quinolinyl, quinoxalinyl, tetrahydroquinolinyl, thiadiazolyl, thiazolyl, and thienyl groups. Unless stated otherwise specifically in the specification, a heteroaryl is optionally substituted by one or more substituents such as those substituents described herein.

Unless stated otherwise, hydrogen atoms are implied in structures depicted herein as necessary to satisfy the valence requirement.

A waved line “ [symbol] ” drawn across a bond or a dashed bond “ [symbol] ” are used interchangeably herein to denote where a bond disconnection or attachment occurs. For example, in the structure

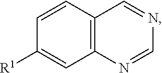

if R¹ is 2-fluoro-6-hydroxyphenyl as in

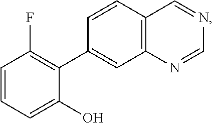 [SMILES: Oc1cccc(F)c1-c1ccc2cncnc2c1]

then R¹ may be depicted as

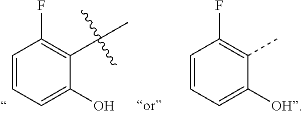

The term “substituted” refers to moieties having substituents replacing a hydrogen on one or more carbons or heteroatoms of the structure. It will be understood that “substitution” or “substituted with” includes the implicit proviso that such substitution is in accordance with permitted valence of the substituted atom and the substituent, and that the substitution results in a stable compound, e.g., which does not spontaneously undergo transformation such as by rearrangement, cyclization, elimination, etc. As used herein, the term “substituted” is contemplated to include all permissible substituents of organic compounds. In a broad aspect, the permissible substituents include acyclic and cyclic, branched and unbranched, carbocyclic and heterocyclic, aromatic and non-aromatic substituents of organic compounds. The permissible substituents can be one or more and the same or different for appropriate organic compounds. For purposes of this disclosure, heteroatoms such as nitrogen may have any permissible substituents of organic compounds described herein which satisfy the valences of the heteroatoms.

A compound disclosed herein, such as a compound of Formula (I) or (II), is optionally substituted by one or more, such as 1, 2 or 3 substituents selected from:

halogen, oxo, —CN, C₁₋₆ alkyl, C₂₋₆ alkenyl, C₂₋₆ alkynyl, 2- to 6-membered heteroalkyl, 2- to 6-membered heteroalkenyl, 2- to 6-membered heteroalkynyl, —C₀₋₆ alkyl-(C₃₋₁₂ carbocycle), -(2- to 6-membered heteroalkyl)-(C₃₋₁₂ carbocycle), —C₀₋₆ alkyl-(3- to 12-membered heterocycle), -(2- to 6-membered heteroalkyl)-(3- to 12-membered heterocycle), —OR²², —SR²², —N(R²²)(R²³), ═NR²², ═C(R²¹)₂, —C(O)OR²², —OC(O)N(R²²)(R²³), —N(R²²)C(O)N(R²²)(R²³), —N(R²²)C(O)OR²², —N(R²²)S(O)₂R²², —C(O)R²², —S(O)R²², —OC(O)R²², —C(O)N(R²²)(R²³), —C(O)C(O)N(R²²)(R²³), —N(R²²)C(O)R²², —S(O)₂R²², —S(O)(NR²²)R²², —S(O)₂N(R²²)(R²³)—, —S(═O)(═NR²²)N(R²²)(R²³), and —OCH₂C(O)OR²²; wherein two substituents attached to the same or adjacent atoms optionally join to form C₃₋₁₂ carbocycle or 3- to 12-membered heterocycle; wherein C₁₋₆ alkyl, C₂₋₆ alkenyl, C₂₋₆ alkynyl, 2- to 6-membered heteroalkyl, 2- to 6-membered heteroalkenyl, 2- to 6-membered heteroalkynyl, —C₀₋₆ alkyl-(C₃₋₁₂ carbocycle), -(2- to 6-membered heteroalkyl)-(C₃₋₁₂ carbocycle), —C₀₋₆ alkyl-(3- to 12-membered heterocycle), -(2- to 6-membered heteroalkyl)-(3- to 12-membered heterocycle), C₃₋₁₂ carbocycle, and 3- to 12-membered heterocycle are optionally substituted with one or more substituents independently selected from halogen, oxo, —CN, C₁₋₆ alkyl, C₁₋₆ haloalkyl, C₁₋₆ alkoxy, C₁₋₆ haloalkoxy, —OR²², —SR²², —N(R²²)(R²³), ═NR²², ═C(R²¹)₂, —C(O)OR²², —OC(O)N(R²²)(R²³), —N(R²²)C(O)N(R²²)(R²³), —N(R²²)C(O)OR²², —N(R²²)S(O)₂R²², —C(O)R²², —S(O)R²², —OC(O)R²², —C(O)N(R²²)(R²³), —C(O)C(O)N(R²²)(R²³), —N(R²²)C(O)R²², —S(O)₂R²², —S(O)(NR²²)R²², —S(O)₂N(R²²)(R²³), and —S(═O)(═NR²²)N(R²²)(R²³);
R²¹ is independently selected at each occurrence from hydrogen, halogen, C₁₋₆ alkyl, C₁₋₆ haloalkyl, —C₀₋₆ alkyl-(C₃₋₁₂ carbocycle), and —C₀₋₆ alkyl-(3- to 12-membered heterocycle), or two R²¹ are taken together with the carbon atom to which they are attached to form C₃₋₁₂ carbocycle or 3- to 12-membered heterocycle, each of which is optionally substituted with one, two, or three substituents independently selected from halogen, C₁₋₃ alkyl, C₁₋₃ haloalkyl, and —OH;
R²² is independently selected at each occurrence from hydrogen, C₁₋₆ alkyl, C₁₋₆ haloalkyl, C₂₋₆ alkenyl, C₂₋₆ alkynyl, —C₀₋₆ alkyl-(C₃₋₁₂ carbocycle), and —C₀₋₆ alkyl-(3- to 12-membered heterocycle), wherein-C₀₋₆ alkyl-(C₃₋₁₂ carbocycle) and —C₀₋₆ alkyl-(3- to 12-membered heterocycle) are optionally substituted with one, two, or three groups independently selected from halogen and C₁₋₆ alkyl; and
R²³ is independently selected at each occurrence from hydrogen and C₁₋₆ alkyl; or R²² and R²³ attached to the same nitrogen atom form 3- to 10 membered heterocycle.

In some embodiments, a compound disclosed herein, such as a compound of Formula (I) or (II), is optionally substituted by one or more, such as 1, 2 or 3 substituents selected from:

halogen, oxo, —CN, C₁₋₆ alkyl, C₂₋₆ alkenyl, C₂₋₆ alkynyl, 2- to 6-membered heteroalkyl, 2- to 6-membered heteroalkenyl, 2- to 6-membered heteroalkynyl, —C₀₋₆ alkyl-(C₃₋₁₂ carbocycle), -(2- to 6-membered heteroalkyl)-(C₃₋₁₂ carbocycle), —C₀₋₆ alkyl-(3- to 12-membered heterocycle), -(2- to 6-membered heteroalkyl)-(3- to 12-membered heterocycle), —OR²², —SR²², —N(R²²)(R²³), —NR²², —C(R²¹)₂, —C(O)OR²², —OC(O)N(R²²)(R²³), —N(R²²)C(O)N(R²²)(R²³), —N(R²²)C(O)OR²², —N(R²²)S(O)₂R²², —C(O)R²², —OC(O)R²², —C(O)N(R²²)(R²³), —C(O)C(O)N(R²²)(R²³), —N(R²²)C(O)R²², —S(O)₂R²², —S(O)(NR²²)R²², and —S(O)₂N(R²²)(R²³)—, wherein C₁₋₆ alkyl, C₂₋₆ alkenyl, C₂₋₆ alkynyl, 2- to 6-membered heteroalkyl, 2- to 6-membered heteroalkenyl, 2- to 6-membered heteroalkynyl, —C₀₋₆ alkyl-(C₃₋₁₂ carbocycle), -(2- to 6-membered heteroalkyl)-(C₃₋₁₂ carbocycle), —C₀₋₆ alkyl-(3- to 12-membered heterocycle), -(2- to 6-membered heteroalkyl)-(3- to 12-membered heterocycle), C₃₋₁₂ carbocycle, and 3- to 12-membered heterocycle are optionally substituted with one or more substituents independently selected from halogen, oxo, —CN, C₁₋₆ alkyl, C₁₋₆ haloalkyl, C₁₋₆ alkoxy, C₁₋₆ haloalkoxy, —OR²², —SR²², —N(R²²)(R²³), ═NR²², and ═C(R²¹)₂;
R²¹ is independently selected at each occurrence from hydrogen, halogen, C₁₋₆ alkyl, and C₁₋₆ haloalkyl;
R²² is independently selected at each occurrence from hydrogen, C₁₋₆ alkyl, C₁₋₆ haloalkyl, C₂₋₆ alkenyl, C₂₋₆ alkynyl, —C₀₋₆ alkyl-(C₃₋₁₂ carbocycle), and —C₀₋₆ alkyl-(3- to 12-membered heterocycle), wherein-C₀₋₆ alkyl-(C₃₋₁₂ carbocycle) and —C₀₋₆ alkyl-(3- to 12-membered heterocycle) are optionally substituted with one, two, or three groups independently selected from halogen and C₁₋₆ alkyl;
R²³ is independently selected at each occurrence from hydrogen and C₁₋₆ alkyl; or R²² and R²³ attached to the same nitrogen atom form 3- to 10 membered heterocycle.

In some embodiments, a compound disclosed herein, such as a compound of Formula (I) or (II), is optionally substituted by one or more, such as 1, 2 or 3 substituents selected from halogen, oxo, ═NH, —CN, —NO₂, C₁₋₆ alkyl, C₂₋₆ alkenyl, C₂₋₆ alkynyl, C₃₋₁₀ carbocycle, —CH₂—(C₃₋₁₀ carbocycle), 3- to 10-membered heterocycle, —CH₂-(3- to 10-membered heterocycle), —OH, —OCH₃, —OCH₂CH₃, —NH₂, —NHCH₃, and —NHCH₂CH₃, wherein C₁₋₆ alkyl, C₂₋₆ alkenyl, C₂₋₆ alkynyl, C₃₋₁₀ carbocycle, —CH₂—(C₃₋₁₀ carbocycle), 3- to 10-membered heterocycle, and —CH₂-(3- to 10-membered heterocycle) are optionally substituted with one, two, or three groups independently selected from halogen, oxo, ═NH, —CN, —NO₂, —CH₃, —CH₂CH₃, —CH(CH₃)₂, —C(CH₃)₃, —OH, —OCH₃, —OCH₂CH₃, —NH₂, —NHCH₃, and —NHCH₂CH₃.

It will be understood by those skilled in the art that substituents can themselves be substituted, if appropriate. Unless specifically stated as “unsubstituted”, references to chemical moieties herein are understood to include substituted variants. For example, reference to a “heteroaryl” group or moiety implicitly includes both substituted and unsubstituted variants.

Where bivalent substituent groups are specified herein by their conventional chemical formulae, written from left to right, they are intended to encompass the isomer that would result from writing the structure from right to left, e.g., —CH₂O— is also intended to encompass —OCH₂—.

“Optional” or “optionally” means that the subsequently described event or circumstances may or may not occur, and that the description includes instances where the event or circumstance occurs and instances in which it does not. For example, an “optionally substituted” group may be either unsubstituted or substituted.

Compounds of the present disclosure also include crystalline and amorphous forms of those compounds, pharmaceutically acceptable salts, and active metabolites having the same type of activity, including, for example, polymorphs, pseudopolymorphs, solvates, hydrates, unsolvated polymorphs (including anhydrates), conformational polymorphs, amorphous forms of the compounds, and mixtures thereof.

The compounds described herein may exhibit their natural isotopic abundance, or one or more of the atoms may be artificially enriched in a particular isotope having the same atomic number, but an atomic mass or mass number different from the atomic mass or mass number predominantly found in nature. All isotopic variations of the compounds of the present disclosure, whether radioactive or not, are encompassed within the scope of the present disclosure. For example, hydrogen has three naturally occurring isotopes, denoted ¹H (protium), ²H (deuterium), and ³H (tritium). Protium is the most abundant isotope of hydrogen in nature. Enriching for deuterium may afford certain therapeutic advantages, such as increased in vivo half-life and/or exposure, or may provide a compound useful for investigating in vivo routes of drug elimination and metabolism. Examples of isotopes that may be incorporated into compounds of the present disclosure include, but are not limited to, ²H, ³H, ¹³C, ¹⁴C, ¹⁵N, ¹⁸O, ¹⁷O, ³⁵S, ³⁶Cl, and ¹⁸F. Of particular interest are compounds of Formula (II) enriched in tritium or carbon-14, which can be used, for example, in tissue distribution studies; compounds of the disclosure enriched in deuterium—especially at a site of metabolism—resulting, for example, in compounds having greater metabolic stability; and compounds of Formula (II) enriched in a positron emitting isotope, such as ¹¹C, ¹⁸F, ¹⁵O and ¹³N, which can be used, for example, in Positron Emission Topography (PET) studies. Isotopically-enriched compounds may be prepared by conventional techniques well known to those skilled in the art.

As used herein, the phrase “of the formula”, “having the formula” or “having the structure” is not intended to be limiting and is used in the same way that the term “comprising” is commonly used. For example, if one structure is depicted, it is understood that all stereoisomer and tautomer forms are encompassed, unless stated otherwise.

Certain compounds described herein contain one or more asymmetric centers and can thus give rise to enantiomers, diastereomers, and other stereoisomeric forms, the asymmetric centers of which can be defined, in terms of absolute stereochemistry, as (R)- or (S)-. In some embodiments, in order to optimize the therapeutic activity of the compounds of the disclosure, e.g., to treat cancer, it may be desirable that the carbon atoms have a particular configuration (e.g., (R,R), (S,S), (S,R), or (R,S)) or are enriched in a stereoisomeric form having such configuration. The compounds of the disclosure may be provided as racemic mixtures. Accordingly, the disclosure relates to racemic mixtures, pure stereoisomers (e.g., enantiomers and diastereomers), stereoisomer-enriched mixtures, and the like, unless otherwise indicated. When a chemical structure is depicted herein without any stereochemistry, it is understood that all possible stereoisomers are encompassed by such structure. Similarly, when a particular stereoisomer is shown or named herein, it will be understood by those skilled in the art that minor amounts of other stereoisomers may be present in the compositions of the disclosure unless otherwise indicated, provided that the utility of the composition as a whole is not eliminated by the presence of such other isomers. Individual stereoisomers may be obtained by numerous methods that are known in the art, including preparation using chiral synthons or chiral reagents, resolution using chiral chromatography using a suitable chiral stationary phase or support, or by chemically converting them into diastereomers, separating the diastereoisomers by conventional means such as chromatography or recrystallization, then regenerating the original stereoisomer.

Additionally, where applicable, all cis-trans or E/Z isomers (geometric isomers), tautomeric forms and topoisomeric forms of the compounds described herein are included with the scope of the disclosure unless otherwise specified.

The term “pharmaceutically acceptable” refers to a material that is not biologically or otherwise unacceptable when used in the subject compositions and methods. For example, the term “pharmaceutically acceptable carrier” refers to a material-such as an adjuvant, excipient, glidant, sweetening agent, diluent, preservative, dye, colorant, flavor enhancer, surfactant, wetting agent, dispersing agent, suspending agent, stabilizer, isotonic agent, solvent or emulsifier—that can be incorporated into a composition and administered to a patient without causing unacceptable biological effects or interacting in an unacceptable manner with other components of the composition. Such pharmaceutically acceptable materials typically have met the required standards of toxicological and manufacturing testing, and include those materials identified as suitable inactive ingredients by the U.S. Food and Drug Administration.

The terms “salt” and “pharmaceutically acceptable salt” refer to a salt prepared from a base or an acid. Pharmaceutically acceptable salts are suitable for administration to a patient, such as a mammal (for example, salts having acceptable mammalian safety for a given dosage regime). Salts can be formed from inorganic bases, organic bases, inorganic acids and organic acids. In addition, when a compound contains both a basic moiety, such as an amine, pyridine or imidazole, and an acidic moiety, such as a carboxylic acid or tetrazole, zwitterions may be formed and are included within the term “salt” as used herein. Preferred pharmaceutically acceptable salts of the compounds described herein are pharmaceutically acceptable acid addition salts and pharmaceutically acceptable base addition salts.

“Pharmaceutically acceptable acid addition salt” refers to those salts which retain the biological effectiveness and properties of the free bases, which are not biologically or otherwise undesirable, and which are formed with inorganic acids such as hydrochloric acid, hydrobromic acid, sulfuric acid, nitric acid, phosphoric acid, hydroiodic acid, hydrofluoric acid, phosphorous acid, and the like. Also included are salts that are formed with organic acids such as aliphatic mono- and dicarboxylic acids, phenyl-substituted alkanoic acids, hydroxy alkanoic acids, alkanedioic acids, aromatic acids, aliphatic and aromatic sulfonic acids, etc., and include, for example, acetic acid, trifluoroacetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid, salicylic acid, and the like. Exemplary salts thus include sulfates, pyrosulfates, bisulfates, sulfites, bisulfites, nitrates, phosphates, monohydrogenphosphates, dihydrogenphosphates, metaphosphates, pyrophosphates, chlorides, bromides, iodides, acetates, trifluoroacetates, propionates, caprylates, isobutyrates, oxalates, malonates, succinate suberates, sebacates, fumarates, maleates, mandelates, benzoates, chlorobenzoates, methylbenzoates, dinitrobenzoates, phthalates, benzenesulfonates, toluenesulfonates, phenylacetates, citrates, lactates, malates, tartrates, methanesulfonates, and the like. Also contemplated are salts of amino acids, such as arginates, gluconates, and galacturonates (see, for example, Berge S. M. et al., “Pharmaceutical Salts,” Journal of Pharmaceutical Science, 66:1-19 (1997)). Acid addition salts of basic compounds are, in some embodiments, prepared by contacting the free base forms with a sufficient amount of the desired acid to produce the salt according to methods and techniques with which a skilled artisan is familiar.

“Pharmaceutically acceptable base addition salt” refers to those salts that retain the biological effectiveness and properties of the free acids, which are not biologically or otherwise undesirable. These salts are prepared from addition of an inorganic base or an organic base to the free acid. Pharmaceutically acceptable base addition salts are, in some embodiments, formed with metals or amines, such as alkali and alkaline earth metals or organic amines. Salts derived from inorganic bases include, but are not limited to, sodium, potassium, lithium, ammonium, calcium, magnesium, iron, zinc, copper, manganese, aluminum salts and the like. Salts derived from organic bases include, but are not limited to, salts of primary, secondary, and tertiary amines, substituted amines including naturally occurring substituted amines, cyclic amines and basic ion exchange resins, for example, isopropylamine, trimethylamine, diethylamine, triethylamine, tripropylamine, ethanolamine, diethanolamine, 2-dimethylaminoethanol, 2-diethylaminoethanol, dicyclohexylamine, lysine, arginine, histidine, caffeine, procaine, N,N-dibenzylethylenediamine, chloroprocaine, hydrabamine, choline, betaine, ethylenediamine, ethylenedianiline, N-methylglucamine, glucosamine, methylglucamine, theobromine, purines, piperazine, piperidine, N-ethylpiperidine, polyamine resins and the like. See Berge et al., supra.

The term “effective amount” or “therapeutically effective amount” refers to the amount of an agent that is sufficient to effect beneficial or desired results. The therapeutically effective amount may vary depending upon one or more of: the subject and disease condition being treated, the weight and age of the subject, the severity of the disease condition, the manner of administration and the like, which can readily be determined by one of ordinary skill in the art. An effective amount of an active agent may be administered in a single dose or in multiple doses. A component may be described herein as having at least an effective amount, or at least an amount effective, such as that associated with a particular goal or purpose, such as any described herein. The term “effective amount” also applies to a dose that will provide an image for detection by an appropriate imaging method. The specific dose may vary depending on one or more of: the particular agent chosen, the dosing regimen to be followed, whether it is administered in combination with other compounds, timing of administration, the tissue to be imaged, and the physical delivery system in which it is carried.

As used herein, “treating” or “treatment” refers to an approach for obtaining beneficial or desired results with respect to a disease, disorder, or medical condition (such as cancer) in a subject, including but not limited to the following: (a) preventing the disease or medical condition from occurring, e.g., preventing the reoccurrence of the disease or medical condition or prophylactic treatment of a subject that is pre-disposed to the disease or medical condition; (b) ameliorating the disease or medical condition, e.g., eliminating or causing regression of the disease or medical condition in a subject; (c) suppressing the disease or medical condition, e.g., slowing or arresting the development of the disease or medical condition in a subject; or (d) alleviating symptoms of the disease or medical condition in a subject. For example, “treating cancer” would include preventing cancer from occurring, ameliorating cancer, suppressing cancer, and alleviating the symptoms of cancer. Also, a therapeutic benefit is achieved with the eradication or amelioration of one or more of the physiological symptoms associated with the underlying disorder such that an improvement is observed in the subject, notwithstanding that the subject may still be afflicted with the underlying disorder.

A “therapeutic effect”, as that term is used herein, encompasses a therapeutic benefit and/or prophylactic benefit as described above. A prophylactic effect includes delaying or eliminating the appearance of a disease or condition, delaying or eliminating the onset of symptoms of a disease or condition, slowing, halting, or reversing the progression of a disease or condition, or any combination thereof.

The terms “antagonist” and “inhibitor” are used interchangeably, and they refer to a compound having the ability to inhibit a biological function (e.g., activity, expression, binding, protein-protein interaction) of a target protein (e.g., K-Ras). Accordingly, the terms “antagonist” and “inhibitor” are defined in the context of the biological role of the target protein. While preferred antagonists herein specifically interact with (e.g., bind to) the target, compounds that inhibit a biological activity of the target protein by interacting with other members of the signal transduction pathway of which the target protein is a member are also specifically included within this definition.

The term “selective inhibition” or “selectively inhibit” refers to the ability of a biologically active agent to preferentially reduce the target signaling activity as compared to off-target signaling activity, via direct or indirect interaction with the target.

The terms “subject” and “patient” refer to an animal, such as a mammal, for example a human. The methods described herein can be useful in both human therapeutics and veterinary applications. In some embodiments, the subject is a mammal, such as a human. “Mammal” includes humans and both domestic animals such as laboratory animals and household pets (e.g., cats, dogs, swine, cattle, sheep, goats, horses, rabbits), and non-domestic animals such as wildlife and the like.

The terms “therapeutic agent”, “therapeutic capable agent” or “treatment agent” are used interchangeably and refer to a molecule or compound that confers some beneficial effect upon administration to a subject. The beneficial effect includes enablement of diagnostic determinations; amelioration of a disease, symptom, disorder, or pathological condition; reducing or preventing the onset of a disease, symptom, disorder or condition; and generally counteracting a disease, symptom, disorder or pathological condition.

The terms “polypeptide”, “peptide” and “protein” are used interchangeably herein to refer to polymers of amino acids of any length. The polymer may be linear or branched, it may comprise modified amino acids, and it may be interrupted by non-amino acids. The terms also encompass an amino acid polymer that has been modified; for example, disulfide bond formation, glycosylation, lipidation, acetylation, phosphorylation, or any other manipulation, such as conjugation with a labeling component. As used herein the term “amino acid” refers to either natural and/or unnatural or synthetic amino acids, including glycine and both the D or L optical isomers, and amino acid analogs and peptidomimetics.

The terms “polynucleotide”, “nucleotide”, “nucleotide sequence”, “nucleic acid” and “oligonucleotide” are used interchangeably. They refer to a polymeric form of nucleotides of any length, either deoxyribonucleotides or ribonucleotides, or analogs thereof. Polynucleotides may have any three-dimensional structure, and may perform any function, known or unknown. The following are non-limiting examples of polynucleotides: coding or non-coding regions of a gene or gene fragment, loci (locus) defined from linkage analysis, exons, introns, messenger RNA (mRNA), transfer RNA, ribosomal RNA, short interfering RNA (siRNA), short-hairpin RNA (shRNA), micro-RNA (miRNA), ribozymes, cDNA, recombinant polynucleotides, branched polynucleotides, plasmids, vectors, isolated DNA of any sequence, isolated RNA of any sequence, nucleic acid probes, and primers. A polynucleotide may comprise one or more modified nucleotides, such as methylated nucleotides and nucleotide analogs, such as peptide nucleic acid (PNA), morpholino and locked nucleic acid (LNA), glycol nucleic acid (GNA), threose nucleic acid (TNA), 2′-fluoro, 2′-OMe, and phosphorothiolated DNA. If present, modifications to the nucleotide structure may be imparted before or after assembly of the polymer. The sequence of nucleotides may be interrupted by non-nucleotide components. A polynucleotide may be further modified after polymerization, such as by conjugation with a labeling component or other conjugation target.

As used herein, “expression” refers to the process by which a polynucleotide is transcribed from a DNA template (such as into an mRNA or other RNA transcript) and/or the process by which a transcribed mRNA is subsequently translated into peptides, polypeptides, or proteins. Transcripts and encoded polypeptides may be collectively referred to as “gene product.” If the polynucleotide is derived from genomic DNA, expression may include splicing of the mRNA in a eukaryotic cell.

An “antigen” is a moiety or molecule that contains an epitope, and, as such, also specifically binds to an antibody. An “antigen binding unit” may be whole or a fragment (or fragments) of a full-length antibody, a structural variant thereof, a functional variant thereof, or a combination thereof. A full-length antibody may be, for example, a monoclonal, recombinant, chimeric, deimmunized, humanized and human antibody. Examples of a fragment of a full-length antibody may include, but are not limited to, variable heavy (VH), variable light (VL), a heavy chain found in camelids, such as camels, llamas, and alpacas (VHH or VHH), a heavy chain found in sharks (V-NAR domain), a single domain antibody (sdAb, e.g., “nanobody”) that comprises a single antigen-binding domain, Fv, Fd, Fab, Fab′, F(ab′)₂, and “r IgG” (or half antibody). Examples of modified fragments of antibodies may include, but are not limited to scFv, di-scFv or bi(s)-scFv, scFv-Fc, scFv-zipper, scFab, Fab2, Fab3, diabodies, single chain diabodies, tandem diabodies (Tandab's), tandem di-scFv, tandem tri-scFv, minibodies (e.g., (VH-VL-CH3)2, (scFv-CH3)2, ((scFv)2-CH3+CH3), ((scFv)2-CH3) or (scFv-CH3-scFv)2), and multibodies (e.g., triabodies or tetrabodies).

The term “antibody” and “antibodies” encompass any antigen binding units, including without limitation: monoclonal antibodies, human antibodies, humanized antibodies, camelised antibodies, chimeric antibodies, and any other epitope-binding fragments.

“Prodrug” is meant to indicate a compound that may be converted under physiological conditions or by solvolysis to a biologically active compound described herein (e.g., a compound of Formula (II)). Thus, the term “prodrug” refers to a precursor of a biologically active compound that is pharmaceutically acceptable. In some aspects, a prodrug is inactive when administered to a subject but is converted in vivo to an active compound, for example, by hydrolysis. The prodrug compound often offers advantages of solubility, tissue compatibility or delayed release in a mammalian organism (see, e.g., Bundgard, H., Design of Prodrugs (1985), pp. 7-9, 21-24 (Elsevier, Amsterdam); Higuchi, T., et al., “Pro-drugs as Novel Delivery Systems,” (1987) A.C.S. Symposium Series, Vol. 14; and Bioreversible Carriers in Drug Design, ed. Edward B. Roche, American Pharmaceutical Association and Pergamon Press each of which is incorporated in full by reference herein). The term “prodrug” is also meant to include any covalently bonded carriers, which release the active compound in vivo when such prodrug is administered to a mammalian subject. Prodrugs of an active compound, as described herein, are typically prepared by modifying functional groups present in the active compound in such a way that the modifications are cleaved, either in routine manipulation or in vivo, to the parent active compound. Prodrugs include compounds wherein a hydroxy, amino or mercapto group is bonded to any group that, when the prodrug of the active compound is administered to a mammalian subject, cleaves to form a free hydroxy, free amino or free mercapto group, respectively. Examples of prodrugs include, but are not limited to, acetate, formate and benzoate derivatives of a hydroxy functional group, or acetamide, formamide and benzamide derivatives of an amine functional group in the active compound, and the like.

The term “in vivo” refers to an event that takes place in a subject's body. The term “ex vivo” refers to an event that first takes place outside of the subject's body for a subsequent in vivo application into a subject's body. For example, an ex vivo preparation may involve preparation of cells outside of a subject's body for the purpose of introduction of the prepared cells into the same or a different subject's body. The term “in vitro” refers to an event that takes place outside of a subject's body. For example, an in vitro assay encompasses any assay run outside of a subject's body. In vitro assays encompass cell-based assays in which cells alive or dead are employed. In vitro assays also encompass a cell-free assay in which no intact cells are employed.

The disclosure is also meant to encompass the in vivo metabolic products of the disclosed compounds. Such products may result from, for example, the oxidation, reduction, hydrolysis, amidation, esterification, and the like of the administered compound, primarily due to enzymatic processes. Accordingly, the disclosure includes compounds produced by a process comprising administering a compound disclosed herein to a mammal for a period of time sufficient to yield a metabolic product thereof. Such products are typically identified by administering a radiolabeled compound of the disclosure in a detectable dose to an animal, such as rat, mouse, guinea pig, monkey, or to a human, allowing sufficient time for metabolism to occur, and isolating its conversion products from the urine, blood or other biological samples.

The term “Ras” or “RAS” refers to a protein in the Rat sarcoma (Ras) superfamily of small GTPases, such as in the Ras subfamily. The Ras superfamily includes, but is not limited to, the Ras subfamily, Rho subfamily, Rab subfamily, Rap subfamily, Arf subfamily, Ran subfamily, Rheb subfamily, RGK subfamily, Rit subfamily, Miro subfamily, and Unclassified subfamily. In some embodiments, a Ras protein is selected from the group consisting of KRAS (also used interchangeably herein as K-Ras, K-ras, or Kras), HRAS (or H-Ras), NRAS (or N-Ras), MRAS (or M-Ras), ERAS (or E-Ras), RRAS2 (or R-Ras2), RALA (or RalA), RALB (or RalB), RIT1, and any combination thereof, such as from KRAS, HRAS, NRAS, RALA, RALB, and any combination thereof. The terms “mutant Ras” and “Ras mutant”, as used interchangeably herein, refer to a Ras protein with one or more amino acid mutations, such as with respect to a common reference sequence such as a wild-type (WT) sequence. In some embodiments, a mutant Ras is selected from a mutant KRAS, mutant HRAS, mutant NRAS, mutant MRAS, mutant ERAS, mutant RRAS2, mutant RALA, mutant RALB, mutant RIT1, and any combination thereof, such as from a mutant KRAS, mutant HRAS, mutant NRAS, mutant RALA, mutant RALB, and any combination thereof. In some embodiments, a mutation can be an introduced mutation, a naturally occurring mutation, or a non-naturally occurring mutation. In some embodiments, a mutation can be a substitution (e.g., a substituted amino acid), insertion (e.g., addition of one or more amino acids), or deletion (e.g., removal of one or more amino acids). In some embodiments, two or more mutations can be consecutive, non-consecutive, or a combination thereof. In some embodiments, a mutation can be present at any position of Ras. In some embodiments, a mutation can be present at position 12, 13, 62, 92, 95, 96 (e.g., Y96D), or any combination thereof of Ras relative to SEQ ID No. 1 when optimally aligned. In some embodiments, a mutant Ras may comprise about or at least about 1, 2, 3, 4, 5, 6, 7, 8, 9, 10, 11, 12, 13, 14, 15, 16, 17, 18, 19, 20, 25, 30, 35, 40, 45, 50, or more than 50 mutations. In some embodiments, a mutant Ras may comprise up to about 1, 2, 3, 4, 5, 6, 7, 8, 9, 10, 11, 12, 13, 14, 15, 16, 17, 18, 19, 20, 25, 30, 35, 40, 45, or 50 mutations. In some embodiments, the mutant Ras is about or up to about 500, 400, 300, 250, 240, 233, 230, 220, 219, 210, 208, 206, 204, 200, 195, 190, 189, 188, 187, 186, 185, 180, 175, 174, 173, 172, 171, 170, 169, 168, 167, 166, 165, 160, 155, 150, 125, 100, 90, 80, 70, 60, 50, or fewer than 50 amino acids in length. In some embodiments, an amino acid of a mutation is a proteinogenic, natural, standard, non-standard, non-canonical, essential, non-essential, or non-natural amino acid. In some embodiments, an amino acid of a mutation has a positively charged side chain, a negatively charged side chain, a polar uncharged side chain, a non-polar side chain, a hydrophobic side chain, a hydrophilic side chain, an aliphatic side chain, an aromatic side chain, a cyclic side chain, an acyclic side chain, a basic side chain, or an acidic side chain. In some embodiments, a mutation comprises a reactive moiety. In some embodiments, a substituted amino acid comprises a reactive moiety. In some embodiments, a mutant Ras can be further modified, such as by conjugation with a detectable label. In some embodiments, a mutant Ras is a full-length or truncated polypeptide. For example, a mutant Ras can be a truncated polypeptide comprising residues 1-169 or residues 11-183 (e.g., residues 11-183 of a mutant RALA or mutant RALB).

As used herein, the term “corresponding to” or “corresponds to” as applied to an amino acid residue in a polypeptide sequence refers to the correspondence of such amino acid relative to a reference sequence when optimally aligned (e.g., taking into consideration of gaps, insertions and mismatches; wherein alignment may be primary sequence alignment or three-dimensional structural alignment of the folded proteins). For instance, the serine residue in a K-Ras G12S mutant refers to the serine corresponding to residue 12 of SEQ ID No. 4, which can serve as a reference sequence. For instance, the aspartate residue in a K-Ras G12D mutant refers to the aspartate corresponding to residue 12 of SEQ ID No. 2, which can serve as a reference sequence. When an amino acid of a mutant Ras protein corresponds to an amino acid position in the WT Ras protein, it will be understood that although the mutant Ras protein amino acid may be a different amino acid (e.g., G12D, wherein the wildtype G at position 12 is replaced by an aspartate at position 12 of SEQ ID. No. 1), the mutant amino acid is at the position corresponding to the wildtype amino acid (e.g., of SEQ ID No. 1). In embodiments, a modified Ras mutant protein disclosed herein may comprise truncations at the C-terminus, or truncations at the N-terminal end preceding the G12S mutant serine residue. The G12S mutant serine residue in such N-terminal truncated modified mutant is still considered corresponding to position 12 of SEQ ID No. 1. In addition, an aspartate residue at position 12 of SEQ ID No. 2 finds a corresponding residue in SEQ ID Nos. 6 and 8.

The terms “Switch II pocket” and “switch II binding pocket,” as used interchangeably herein, refer to a binding pocket formed under the “Switch II” loop of Ras. In some embodiments, the Switch II pocket is located between the central beta-sheet (β-sheet) of Ras and the alpha(α)₂- and α3-helices. In some embodiments, the Switch II binding pocket is located about or at least about 5, 10, 20, 30, 40, 50, 60, 70, 80, 90, 100, 110, 120, 130, 140, 150, 160, 170, 180, 190, 200 nanometers (nm), or more from position 12, position 60, position 99, or any combination thereof. In some embodiments, the Switch II binding pocket is located up to about 5, 10, 20, 30, 40, 50, 60, 70, 80, 90, 100, 110, 120, 130, 140, 150, 160, 170, 180, 190, 200 nm, or more from position 12, position 60, position 99, or any combination thereof. In some cases, the Switch II pocket may be formed upon binding to a small molecule (e.g., a small molecule inhibitor). Alternatively, the Switch II pocket may be formed prior to binding to a small molecule.

In some embodiments, the Switch II pocket of Ras comprises three, four, five, six, seven, eight, nine, ten, eleven, twelve, thirteen, fourteen, fifteen or more amino acid residues, or between three to fifteen residues selected from the group consisting of V7, V9, G10, G12, G12X mutant (e.g., G12C, G12S, G12D, or G12V), K14, K16, P34, T58, A59, G60, Q61, E62, E63, Y64, S65, R⁶⁸, D69, M72, D92, H95, Y96, Q99, 1100, R¹⁰², and V103 of SEQ ID NO. 1, or corresponding amino acid residues of an HRAS or NRAS protein. In some embodiments, the Switch II pocket of Ras comprises three, four, five, six, seven, eight, nine, ten, eleven, or twelve amino acid residues selected from the group consisting of G10, G12, G12X mutant (e.g., G12C, G12S, G12D, or G12V), K16, P34, T58, A59, E62, R⁶⁸, D69, H95, Q99, R¹⁰², and V103 of SEQ ID NO. 1 or corresponding amino acid residues of an HRAS or NRAS protein.

The term “leaving group” is used herein in accordance with its well understood meaning in Chemistry and refers to an atom or group of atoms which breaks away from the rest of the molecule, taking with it the electron pair which used to be the bond between the leaving group and the rest of the molecule.

A “degradation enhancer” is a compound capable of binding a ubiquitin ligase protein (e.g., E3 ubiquitin ligase protein) or a compound capable of binding a protein that is capable of binding to a ubiquitin ligase protein to form a protein complex capable of conjugating a ubiquitin protein to a target protein. In embodiments, the degradation enhancer is capable of binding to an E3 ubiquitin ligase protein or a protein complex comprising an E3 ubiquitin ligase protein. In embodiments, the degradation enhancer is capable of binding to an E2 ubiquitin-conjugating enzyme. In embodiments, the degradation enhancer is capable of binding to a protein complex comprising an E2 ubiquitin-conjugating enzyme and an E3 ubiquitin ligase protein.

### Modified Proteins and Compounds

In certain aspects, the present disclosure provides a modified Ras protein comprising a compound covalently bonded to an amino acid residue of said Ras protein, wherein the modified Ras protein comprises a compound of Formula (I′):

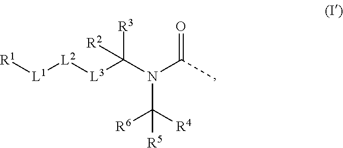

wherein:
the dashed line represents the covalent bond to the amino acid residue;
R¹ is selected from C₃₋₁₂ carbocycle and 3- to 12-membered heterocycle, each of which is optionally substituted with one or more R²⁰;
L¹ is 5- to 20-membered heterocycle optionally substituted with one or more R²⁰;
L² is selected from a bond, C₁₋₄ alkylene, 2- to 4-membered heteroalkylene, —O—, —N(R¹⁹)—, —C(O)—, —S—, —S(O)₂—, —S(O)—, —P(O)R¹⁹, —N(R¹⁹)S(O)₂—, —N(R¹⁹)S(O)—, —N(R¹⁹)P(O)R¹⁹—, —S(O)₂N(R¹⁹)—, —S(O)N(R¹⁹)—, —P(O)R¹⁹N(R¹⁹)—, —OS(O)₂—, —OS(O)—, —OP(O)R¹⁹—, —S(O)₂O—, —S(O)O—, and —P(O)R¹⁹O—, wherein C₁₋₄ alkylene and 2- to 4-membered heteroalkylene are optionally substituted with one, two, or three R²⁰;
   L³ and R², together with the atoms to which they are attached, form 3- to 8-membered monocyclic heterocycloalkyl, C₃₋₈ monocyclic cycloalkyl, 7- to 12-membered spirocyclic heterocycloalkyl, or 7- to 12-membered fused bicyclic heterocycloalkyl, each of which is optionally substituted with one, two, or three R²⁰;
   L³ and R⁶, together with the atoms to which they are attached, form 4- to 8-membered monocyclic heterocycloalkyl, 7- to 12-membered spirocyclic heterocycloalkyl, or 7- to 12-membered fused bicyclic heterocycloalkyl, each of which is optionally substituted with one, two, or three R²⁰, wherein the 4- to 8-membered monocyclic heterocycloalkyl formed by L³ and R⁶ is not piperazine; or
   R² and R⁶, together with the atoms to which they are attached, form 3- to 8-membered monocyclic heterocycloalkyl optionally substituted with one, two, or three R²⁰, wherein L³ is a bond;
R² is selected from R²⁰, or R² and R³, together with the carbon atom to which they are attached, form C₃₋₆ cycloalkyl optionally substituted with one, two, or three R²⁰;
R³ is selected from hydrogen and R²⁰;
R⁴, R⁵, and R⁶ are each independently selected from hydrogen and R²⁰, or R⁴ and R⁵, together with the carbon atom to which they are attached, form C₃₋₆ cycloalkyl optionally substituted with one, two, or three R²⁰;
R¹⁹ is independently selected at each occurrence from hydrogen, —CN, C₁₋₆ alkyl, C₂₋₆ alkenyl, C₂₋₆ alkynyl, —C₀₋₆ alkyl-(C₃₋₁₂ carbocycle), —C₀₋₆ alkyl-(3- to 12-membered heterocycle), —OH, and —O(C₁₋₆ alkyl), wherein C₁₋₆ alkyl, C₂₋₆ alkenyl, C₂₋₆ alkynyl, —C₀₋₆ alkyl-(C₃₋₁₂ carbocycle), —C₀₋₆ alkyl-(3- to 12-membered heterocycle), and —O(C₁₋₆ alkyl) are optionally substituted with one, two, or three R²⁰;
R²⁰ is independently selected at each occurrence from halogen, oxo, —CN, C₁₋₆ alkyl, C₂₋₆ alkenyl, C₂₋₆ alkynyl, 2- to 6-membered heteroalkyl, 2- to 6-membered heteroalkenyl, 2- to 6-membered heteroalkynyl, —C₀₋₆ alkyl-(C₃₋₁₂ carbocycle), -(2- to 6-membered heteroalkyl)-(C₃₋₁₂ carbocycle), —C₀₋₆ alkyl-(3- to 12-membered heterocycle), -(2- to 6-membered heteroalkyl)-(3- to 12-membered heterocycle), —OR²², —SR²², —N(R²²)(R²³), ═NR²², ═C(R²¹)₂, —C(O)OR²², —OC(O)N(R²²)(R²³), —N(R²²)C(O)N(R²²)(R²³), —N(R²²)C(O)OR²², —N(R²²)S(O)₂R²², —C(O)R²², —S(O)R²², —OC(O)R²², —C(O)N(R²²)(R²³), —C(O)C(O)N(R²²)(R²³), —N(R²²)C(O)R²², —S(O)₂R²², —S(O)(NR²²)R²², —S(O)₂N(R²²)(R²³)—, —S(—O)(—NR²²)N(R²²)(R²³), and —OCH₂C(O)OR²²; wherein two R²⁰ attached to the same or adjacent atoms optionally join to form C₃₋₁₂ carbocycle or 3- to 12-membered heterocycle; wherein C₁₋₆ alkyl, C₂₋₆ alkenyl, C₂₋₆ alkynyl, 2- to 6-membered heteroalkyl, 2- to 6-membered heteroalkenyl, 2- to 6-membered heteroalkynyl, —C₀₋₆ alkyl-(C₃₋₁₂ carbocycle), -(2- to 6-membered heteroalkyl)-(C₃₋₁₂ carbocycle), —C₀₋₆ alkyl-(3- to 12-membered heterocycle), -(2- to 6-membered heteroalkyl)-(3- to 12-membered heterocycle), C₃₋₁₂ carbocycle, and 3- to 12-membered heterocycle are optionally substituted with one or more substituents independently selected from halogen, oxo, —CN, C₁₋₆ alkyl, C₁₋₆ haloalkyl, C₁₋₆ alkoxy, C₁₋₆ haloalkoxy, —OR²², —SR²², —N(R²²)(R²³), ═NR²², ═C(R²¹)₂, —C(O)OR²², —OC(O)N(R²²)(R²³), —N(R²²)C(O)N(R²²)(R²³), —N(R²²)C(O)OR²², —N(R²²)S(O)₂R²², —C(O)R²², —S(O)R²², —OC(O)R²², —C(O)N(R²²)(R²³), —C(O)C(O)N(R²²)(R²³), —N(R²²)C(O)R²², —S(O)₂R²², —S(O)(NR²²)R²², —S(O)₂N(R²²)(R²³), and —S(═O)(═NR²²)N(R²²)(R²³);
R²¹ is independently selected at each occurrence from hydrogen, halogen, C₁₋₆ alkyl, C₂₋₆ alkenyl, C₂₋₆ alkynyl, —C₀₋₆ alkyl-(C₃₋₁₂ carbocycle), and —C₀₋₆ alkyl-(3- to 12-membered heterocycle), or two R²¹ are taken together with the carbon atom to which they are attached to form C₃₋₁₂ carbocycle or 3- to 12-membered heterocycle, wherein C₁₋₆ alkyl, C₂₋₆ alkenyl, C₂₋₆ alkynyl, —C₀₋₆ alkyl-(C₃₋₁₂ carbocycle), —C₀₋₆ alkyl-(3- to 12-membered heterocycle), C₃₋₁₂ carbocycle, and 3- to 12-membered heterocycle are optionally substituted with one, two, or three R²⁰;
R²² is independently selected at each occurrence from hydrogen, C₁₋₆ alkyl, C₂₋₆ alkenyl, C₂₋₆ alkynyl, —C₀₋₆ alkyl-(C₃₋₁₂ carbocycle), and —C₀₋₆ alkyl-(3- to 12-membered heterocycle), wherein C₁₋₆ alkyl, C₂₋₆ alkenyl, C₂₋₆ alkynyl, —C₀₋₆ alkyl-(C₃₋₁₂ carbocycle), and —C₀₋₆ alkyl-(3- to 12-membered heterocycle) are optionally substituted with one, two, or three R²⁰; and
R²³ is independently selected at each occurrence from hydrogen, C₁₋₆ alkyl, and C₁₋₆ haloalkyl; or R²² and R²³ attached to the same nitrogen atom form 3- to 10-membered heterocycle optionally substituted with one, two, or three R²⁰.

In certain aspects, the present disclosure provides a modified Ras protein comprising a compound covalently bonded to an amino acid residue of said Ras protein, wherein the modified Ras protein comprises a compound of Formula (I′):

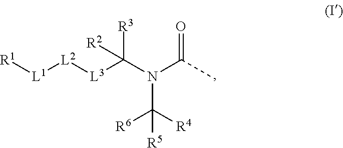

wherein:
the dashed line represents the covalent bond to the amino acid residue;
R¹ is selected from C₃₋₁₂ carbocycle and 3- to 12-membered heterocycle, each of which is optionally substituted with one or more R²⁰;
L¹ is 5- to 20-membered heterocycle optionally substituted with one or more R²⁰;
L² is selected from a bond, C₁₋₄ alkylene, 2- to 4-membered heteroalkylene, —O—, —N(R¹⁹)—, —C(O)—, —S—, —S(O)₂—, —S(O)—, —P(O)R¹⁹—, —N(R¹⁹)S(O)₂—, —N(R¹⁹)S(O)—, —N(R¹⁹)P(O)R¹⁹—, —S(O)₂N(R¹⁹)—, —S(O)N(R¹⁹)—, —P(O)R¹⁹N(R¹⁹)—, —OS(O)₂—, —OS(O)—, —OP(O)R¹⁹—, —S(O)₂O—, —S(O)O—, and —P(O)R¹⁹O—, wherein C₁₋₄ alkylene and 2- to 4-membered heteroalkylene are optionally substituted with one, two, or three R²⁰;
   L³ and R², together with the atoms to which they are attached, form 3- to 8-membered monocyclic heterocycloalkyl, C₃₋₈ monocyclic cycloalkyl, 7- to 12-membered spirocyclic heterocycloalkyl, or 7- to 12-membered fused bicyclic heterocycloalkyl, each of which is optionally substituted with one, two, or three substituents selected from halogen, —OH, —CN, C₁₋₆ alkyl, C₃₋₆ cycloalkyl, —O(C₁₋₆ alkyl), and —O(C₃₋₆ cycloalkyl), wherein C₁₋₆ alkyl, C₃₋₆ cycloalkyl, —O(C₁₋₆ alkyl), and —O(C₃₋₆ cycloalkyl) are optionally substituted with one, two, or three substituents selected from halogen, —OH, —CN, C₁₋₃ alkyl, C₁₋₃ haloalkyl, C₃₋₆ cycloalkyl, —O(C₁₋₃ alkyl), and —O(C₁₋₃ haloalkyl);
   L³ and R⁶, together with the atoms to which they are attached, form 4- to 8-membered monocyclic heterocycloalkyl, 7- to 12-membered spirocyclic heterocycloalkyl, or 7- to 12-membered fused bicyclic heterocycloalkyl, each of which is optionally substituted with one, two, or three substituents selected from halogen, —OH, —CN, C₁₋₆ alkyl, C₃₋₆ cycloalkyl, —O(C₁₋₆ alkyl), and —O(C₃₋₆ cycloalkyl), wherein C₁₋₆ alkyl, C₃₋₆ cycloalkyl, —O(C₁₋₆ alkyl), and —O(C₃₋₆ cycloalkyl) are optionally substituted with one, two, or three substituents selected from halogen, —OH, —CN, C₁₋₃ alkyl, C₁₋₃ haloalkyl, C₃₋₆ cycloalkyl, —O(C₁₋₃ alkyl), and —O(C₁₋₃ haloalkyl), and wherein the 4- to 8-membered monocyclic heterocycloalkyl formed by L³ and R⁶ is not piperazine; or
   R² and R⁶, together with the atoms to which they are attached, form 3- to 8-membered monocyclic heterocycloalkyl optionally substituted with one, two, or three substituents selected from halogen, —OH, —CN, C₁₋₆ alkyl, C₃₋₆ cycloalkyl, —O(C₁₋₆ alkyl), and —O(C₃₋₆ cycloalkyl), wherein C₁₋₆ alkyl, C₃₋₆ cycloalkyl, —O(C₁₋₆ alkyl), and —O(C₃₋₆ cycloalkyl) are optionally substituted with one, two, or three substituents selected from halogen, —OH, —CN, C₁₋₃ alkyl, C₁₋₃ haloalkyl, C₃₋₆ cycloalkyl, —O(C₁₋₃ alkyl), and —O(C₁₋₃ haloalkyl), and wherein L³ is a bond;
R² is selected from halogen, —CN, C₁₋₆ alkyl, and C₃₋₆ cycloalkyl, or R² and R³, together with the carbon atom to which they are attached, form C₃₋₆ cycloalkyl, wherein C₁₋₆ alkyl and C₃₋₆ cycloalkyl are optionally substituted with one, two, or three substituents selected from halogen, —CN, C₁₋₆ alkyl, C₁₋₆ haloalkyl, C₃₋₆ cycloalkyl, —O(C₁₋₆ alkyl), and —O(C₁₋₆ haloalkyl);
R³ is selected from hydrogen, halogen, —CN, C₁₋₆ alkyl, and C₃₋₆ cycloalkyl, wherein C₁₋₆ alkyl and C₃₋₆ cycloalkyl are optionally substituted with one, two, or three substituents selected from halogen, —CN, C₁₋₆ alkyl, C₁₋₆ haloalkyl, C₃₋₆ cycloalkyl, —O(C₁₋₆ alkyl), and —O(C₁₋₆ haloalkyl);
R⁴, R⁵, and R⁶ are each independently selected from hydrogen, halogen, —CN, C₁₋₆ alkyl, and C₃₋₆ cycloalkyl, or R⁴ and R⁵, together with the carbon atom to which they are attached, form C₃₋₆ cycloalkyl, wherein C₁₋₆ alkyl and C₃₋₆ cycloalkyl are optionally substituted with one, two, or three substituents selected from halogen, —CN, C₁₋₆ alkyl, C₁₋₆ haloalkyl, C₃₋₆ cycloalkyl, —O(C₁₋₆ alkyl), and —O(C₁₋₆ haloalkyl);
R¹⁹ is independently selected at each occurrence from hydrogen, —CN, C₁₋₆ alkyl, C₃₋₆ carbocycle, 3- to 6-membered heterocycle, —OH, —O(C₁₋₆ alkyl), and —O(C₁₋₆ haloalkyl);
R²⁰ is independently selected at each occurrence from halogen, oxo, —CN, C₁₋₆ alkyl, C₂₋₆ alkenyl, C₂₋₆ alkynyl, 2- to 6-membered heteroalkyl, 2- to 6-membered heteroalkenyl, 2- to 6-membered heteroalkynyl, —C₀₋₆ alkyl-(C₃₋₁₂ carbocycle), -(2- to 6-membered heteroalkyl)-(C₃₋₁₂ carbocycle), —C₀₋₆ alkyl-(3- to 12-membered heterocycle), -(2- to 6-membered heteroalkyl)-(3- to 12-membered heterocycle), —OR²², —SR²², —N(R²²)(R²³), ═NR²², —C(R²¹)₂, —C(O)OR²², —OC(O)N(R²²)(R²³), —N(R²²)C(O)N(R²²)(R²³), —N(R²²)C(O)OR²², —N(R²²)S(O)₂R²², —C(O)R²², —S(O)R²², —OC(O)R²², —C(O)N(R²²)(R²³), —C(O)C(O)N(R²²)(R²³), —N(R²²)C(O)R²², —S(O)₂R²², —S(O)(NR²²)R²², —S(O)₂N(R²²)(R²³)—, —S(—O) (—NR²²)N(R²²)(R²³), and —OCH₂C(O)OR²²; wherein two R²⁰ attached to the same or adjacent atoms optionally join to form C₃₋₁₂ carbocycle or 3- to 12-membered heterocycle; wherein C₁₋₆ alkyl, C₂₋₆ alkenyl, C₂₋₆ alkynyl, 2- to 6-membered heteroalkyl, 2- to 6-membered heteroalkenyl, 2- to 6-membered heteroalkynyl, —C₀₋₆ alkyl-(C₃₋₁₂ carbocycle), -(2- to 6-membered heteroalkyl)-(C₃₋₁₂ carbocycle), —C₀₋₆ alkyl-(3- to 12-membered heterocycle), -(2- to 6-membered heteroalkyl)-(3- to 12-membered heterocycle), C₃₋₁₂ carbocycle, and 3- to 12-membered heterocycle are optionally substituted with one or more substituents independently selected from halogen, oxo, —CN, C₁₋₆ alkyl, C₁₋₆ haloalkyl, C₁₋₆ alkoxy, C₁₋₆ haloalkoxy, —OR²², —SR²², —N(R²²)(R²³), ═NR²², ═C(R²¹)₂, —C(O)OR²², —OC(O)N(R²²)(R²³), —N(R²²)C(O)N(R²²)(R²³), —N(R²²)C(O)OR²², —N(R²²)S(O)₂R²², —C(O)R²², —S(O)R²², —OC(O)R²², —C(O)N(R²²)(R²³), —C(O)C(O)N(R²²)(R²³), —N(R²²)C(O)R²², —S(O)₂R²², —S(O)(NR²²)R²², —S(O)₂N(R²²)(R²³), and —S(═O)(═NR²²)N(R²²)(R²³);
R²¹ is independently selected at each occurrence from hydrogen, halogen, C₁₋₆ alkyl, C₁₋₆ haloalkyl, —C₀₋₆ alkyl-(C₃₋₁₂ carbocycle), and —C₀₋₆ alkyl-(3- to 12-membered heterocycle), or two R²¹ are taken together with the carbon atom to which they are attached to form C₃₋₁₂ carbocycle or 3- to 12-membered heterocycle, each of which is optionally substituted with one, two, or three substituents independently selected from halogen, C₁₋₃ alkyl, C₁₋₃ haloalkyl, and —OH;
R²² is independently selected at each occurrence from hydrogen, C₁₋₆ alkyl, C₁₋₆ haloalkyl, —C₀₋₆ alkyl-(C₃₋₁₂ carbocycle), and —C₀₋₆ alkyl-(3- to 12-membered heterocycle); and
R²³ is independently selected at each occurrence from hydrogen and C₁₋₆ alkyl; or R²² and R²³ attached to the same nitrogen atom form 3- to 10 membered heterocycle.

In some embodiments, the modified protein of Formula (I′) is a modified protein of Formula (I′-a), (I′-b), (I′-c), or (I′-d):

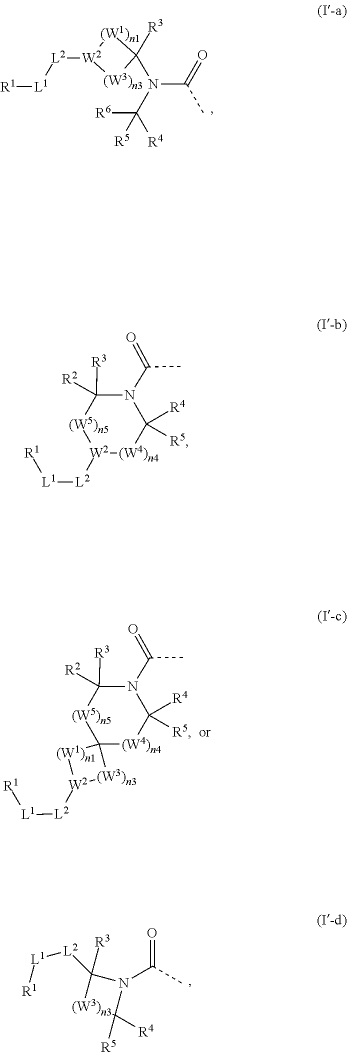

wherein:
W¹, W³, W⁴, and W⁵ are each independently selected from N(R¹⁰), C(R¹¹)₂, C(O), O, S(O), and S(O)₂;
W² is selected from N and C(R¹¹);
n1 and n3 are each independently selected from 0, 1, 2, 3, 4, and 5;
n4 and n5 are each independently selected from 0, 1, 2, 3, 4, and 5;
R¹⁰ is independently selected at each occurrence from hydrogen, C₁₋₆ alkyl, and C₃₋₆ cycloalkyl, wherein C₁₋₆ alkyl and C₃₋₆ cycloalkyl are optionally substituted with one, two, or three substituents selected from halogen, —OH, —CN, C₁₋₃ alkyl, C₁₋₃ haloalkyl, C₃₋₆ cycloalkyl, —O(C₁₋₃ alkyl), and —O(C₁₋₃ haloalkyl); and
R¹¹ is independently selected at each occurrence from hydrogen, halogen, —OH, —CN, C₁₋₆ alkyl, C₃₋₆ cycloalkyl, —O(C₁₋₆ alkyl), and —O(C₃₋₆ cycloalkyl), or two R¹¹ attached to the same carbon atom form C₃₋₆ cycloalkyl, wherein C₁₋₆ alkyl, C₃₋₆ cycloalkyl, —O(C₁₋₆ alkyl), and —O(C₃₋₆ cycloalkyl) are optionally substituted with one, two, or three substituents selected from halogen, —OH, —CN, C₁₋₃ alkyl, C₁₋₃ haloalkyl, C₃₋₆ cycloalkyl, —O(C₁₋₃ alkyl), and —O(C₁₋₃ haloalkyl).

In some embodiments, for a compound of Formula (I′), the amino acid is selected from serine, tyrosine, cysteine, lysine, and histidine. In some embodiments, the amino acid is serine. In some embodiments, the amino acid is tyrosine. In some embodiments, the amino acid is cysteine. In some embodiments, the amino acid is lysine. In some embodiments, the amino acid is histidine. In some embodiments, the amino acid is serine or cysteine.

In certain aspects, the present disclosure provides a modified human K-Ras protein comprising a compound covalently bonded to a serine residue having the structure of Formula (I), wherein the serine residue corresponds to position 12 of SEQ ID No. 4:

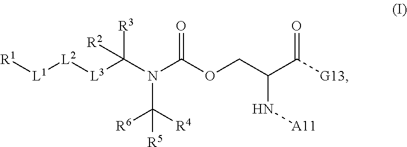

wherein:
the dashed lines represent bonds between the serine residue and alanine 11 and glycine 13 of the K-Ras mutant protein, respectively;
R¹ is selected from C₃₋₁₂ carbocycle and 3- to 12-membered heterocycle, each of which is optionally substituted with one or more R²⁰;
L¹ is 5- to 20-membered heterocycle optionally substituted with one or more R²⁰;
L² is selected from a bond, C₁₋₄ alkylene, 2- to 4-membered heteroalkylene, —O—, —N(R¹⁹)—, —C(O)—, —S—, —S(O)₂—, —S(O)—, —P(O)R¹⁹—, —N(R¹⁹)S(O)₂—, —N(R¹⁹)S(O)—, —N(R¹⁹)P(O)R¹⁹—, —S(O)₂N(R¹⁹)—, —S(O)N(R¹⁹)—, —P(O)R¹°N(R¹⁹)—, —OS(O)₂—, —OS(O)—, —OP(O)R¹⁹—, —S(O)₂O—, —S(O)O—, and —P(O)R¹⁹O—, wherein C₁₋₄ alkylene and 2- to 4-membered heteroalkylene are optionally substituted with one, two, or three R²⁰;
L³ and R², together with the atoms to which they are attached, form 3- to 8-membered monocyclic heterocycloalkyl, C₃₋₈ monocyclic cycloalkyl, 7- to 12-membered spirocyclic heterocycloalkyl, or 7- to 12-membered fused bicyclic heterocycloalkyl, each of which is optionally substituted with one, two, or three R²⁰;
   L³ and R⁶, together with the atoms to which they are attached, form 4- to 8-membered monocyclic heterocycloalkyl, 7- to 12-membered spirocyclic heterocycloalkyl, or 7- to 12-membered fused bicyclic heterocycloalkyl, each of which is optionally substituted with one, two, or three R²⁰, wherein the 4- to 8-membered monocyclic heterocycloalkyl formed by L³ and R⁶ is not piperazine; or
   R² and R⁶, together with the atoms to which they are attached, form 3- to 8-membered monocyclic heterocycloalkyl optionally substituted with one, two, or three R²⁰, wherein L³ is a bond;
R² is selected from R²⁰, or R² and R³, together with the carbon atom to which they are attached, form C₃₋₆ cycloalkyl optionally substituted with one, two, or three R²⁰;
R³ is selected from hydrogen and R²⁰;
R⁴, R⁵, and R⁶ are each independently selected from hydrogen and R²⁰, or R⁴ and R⁵, together with the carbon atom to which they are attached, form C₃₋₆ cycloalkyl optionally substituted with one, two, or three R²⁰;
R¹⁹ is independently selected at each occurrence from hydrogen, —CN, C₁₋₆ alkyl, C₂₋₆ alkenyl, C₂₋₆ alkynyl, —C₀₋₆ alkyl-(C₃₋₁₂ carbocycle), —C₀₋₆ alkyl-(3- to 12-membered heterocycle), —OH, and —O(C₁₋₆ alkyl), wherein C₁₋₆ alkyl, C₂₋₆ alkenyl, C₂₋₆ alkynyl, —C₀₋₆ alkyl-(C₃₋₁₂ carbocycle), —C₀₋₆ alkyl-(3- to 12-membered heterocycle), and —O(C₁₋₆ alkyl) are optionally substituted with one, two, or three R²⁰;
R²⁰ is independently selected at each occurrence from halogen, oxo, —CN, C₁₋₆ alkyl, C₂₋₆ alkenyl, C₂₋₆ alkynyl, 2- to 6-membered heteroalkyl, 2- to 6-membered heteroalkenyl, 2- to 6-membered heteroalkynyl, —C₀₋₆ alkyl-(C₃₋₁₂ carbocycle), -(2- to 6-membered heteroalkyl)-(C₃₋₁₂ carbocycle), —C₀₋₆ alkyl-(3- to 12-membered heterocycle), -(2- to 6-membered heteroalkyl)-(3- to 12-membered heterocycle), —OR²², —SR²², —N(R²²)(R²³), ═NR²², ═C(R²¹)₂, —C(O)OR²², —OC(O)N(R²²)(R²³), —N(R²²)C(O)N(R²²)(R²³), —N(R²²)C(O)OR²², —N(R²²)S(O)₂R²², —C(O)R²², —S(O)R²², —OC(O)R²², —C(O)N(R²²)(R²³), —C(O)C(O)N(R²²)(R²³), —N(R²²)C(O)R²², —S(O)₂R²², —S(O)(NR²²)R²², —S(O)₂N(R²²)(R²³)—, —S(═O)(—NR²²)N(R²²)(R²³), and —OCH₂C(O)OR²²; wherein two R²⁰ attached to the same or adjacent atoms optionally join to form C₃₋₁₂ carbocycle or 3- to 12-membered heterocycle; wherein C₁₋₆ alkyl, C₂₋₆ alkenyl, C₂₋₆ alkynyl, 2- to 6-membered heteroalkyl, 2- to 6-membered heteroalkenyl, 2- to 6-membered heteroalkynyl, —C₀₋₆ alkyl-(C₃₋₁₂ carbocycle), -(2- to 6-membered heteroalkyl)-(C₃₋₁₂ carbocycle), —C₀₋₆ alkyl-(3- to 12-membered heterocycle), -(2- to 6-membered heteroalkyl)-(3- to 12-membered heterocycle), C₃₋₁₂ carbocycle, and 3- to 12-membered heterocycle are optionally substituted with one or more substituents independently selected from halogen, oxo, —CN, C₁₋₆ alkyl, C₁₋₆ haloalkyl, C₁₋₆ alkoxy, C₁₋₆ haloalkoxy, —OR²², —SR²², —N(R²²)(R²³), ═NR²², —C(R²¹)₂, —C(O)OR²², —OC(O)N(R²²)(R²³), —N(R²²)C(O)N(R²²)(R²³), —N(R²²)C(O)OR²², —N(R²²)S(O)₂R²², —C(O)R²², —S(O)R²², —OC(O)R²², —C(O)N(R²²)(R²³), —C(O)C(O)N(R²²)(R²³), —N(R²²)C(O)R²², —S(O)₂R²², —S(O)(NR²²)R²², —S(O)₂N(R²²)(R²³), and —S(═O)(═NR²²)N(R²²)(R²³);
R²¹ is independently selected at each occurrence from hydrogen, halogen, C₁₋₆ alkyl, C₂₋₆ alkenyl, C₂₋₆ alkynyl, —C₀₋₆ alkyl-(C₃₋₁₂ carbocycle), and —C₀₋₆ alkyl-(3- to 12-membered heterocycle), or two R²¹ are taken together with the carbon atom to which they are attached to form C₃₋₁₂ carbocycle or 3- to 12-membered heterocycle, wherein C₁₋₆ alkyl, C₂₋₆ alkenyl, C₂₋₆ alkynyl, —C₀₋₆ alkyl-(C₃₋₁₂ carbocycle), —C₀₋₆ alkyl-(3- to 12-membered heterocycle), C₃₋₁₂ carbocycle, and 3- to 12-membered heterocycle are optionally substituted with one, two, or three R²⁰;
R²² is independently selected at each occurrence from hydrogen, C₁₋₆ alkyl, C₂₋₆ alkenyl, C₂₋₆ alkynyl, —C₀₋₆ alkyl-(C₃₋₁₂ carbocycle), and —C₀₋₆ alkyl-(3- to 12-membered heterocycle), wherein C₁₋₆ alkyl, C₂₋₆ alkenyl, C₂₋₆ alkynyl, —C₀₋₆ alkyl-(C₃₋₁₂ carbocycle), and —C₀₋₆ alkyl-(3- to 12-membered heterocycle) are optionally substituted with one, two, or three R²⁰; and
R²³ is independently selected at each occurrence from hydrogen, C₁₋₆ alkyl, and C₁₋₆ haloalkyl; or R²² and R²³ attached to the same nitrogen atom form 3- to 10-membered heterocycle optionally substituted with one, two, or three R²⁰.

In certain aspects, the present disclosure provides a modified human K-Ras protein comprising a compound covalently bonded to a serine residue having the structure of Formula (I), wherein the serine residue corresponds to position 12 of SEQ ID No. 4:

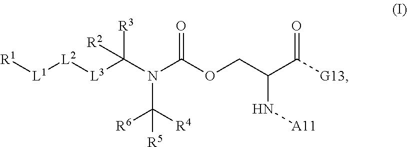

wherein:
the dashed lines represent bonds between the serine residue and alanine 11 and glycine 13 of the K-Ras mutant protein, respectively;
R¹ is selected from C₃₋₁₂ carbocycle and 3- to 12-membered heterocycle, each of which is optionally substituted with one or more R²⁰;
L¹ is 5- to 20-membered heterocycle optionally substituted with one or more R²⁰;
L² is selected from a bond, C₁₋₄ alkylene, 2- to 4-membered heteroalkylene, —O—, —N(R¹⁹)—, —C(O)—, —S—, —S(O)₂—, —S(O)—, —P(O)R¹⁹—, —N(R¹⁹)S(O)₂—, —N(R¹⁹)S(O)—, —N(R¹⁹)P(O)R¹⁹—, —S(O)₂N(R¹⁹)—, —S(O)N(R¹⁹)—, —P(O)R¹⁹N(R¹⁹)—, —OS(O)₂—, —OS(O)—, —OP(O)R¹⁹—, —S(O)₂O—, —S(O)O—, and —P(O)R¹⁹O—, wherein C₁₋₄ alkylene and 2- to 4-membered heteroalkylene are optionally substituted with one, two, or three R²⁰;
   L³ and R², together with the atoms to which they are attached, form 3- to 8-membered monocyclic heterocycloalkyl, C₃₋₈ monocyclic cycloalkyl, 7- to 12-membered spirocyclic heterocycloalkyl, or 7- to 12-membered fused bicyclic heterocycloalkyl, each of which is optionally substituted with one, two, or three substituents selected from halogen, —OH, —CN, C₁₋₆ alkyl, C₃₋₆ cycloalkyl, —O(C₁₋₆ alkyl), and —O(C₃₋₆ cycloalkyl), wherein C₁₋₆ alkyl, C₃₋₆ cycloalkyl, —O(C₁₋₆ alkyl), and —O(C₃₋₆ cycloalkyl) are optionally substituted with one, two, or three substituents selected from halogen, —OH, —CN, C₁₋₃ alkyl, C₁₋₃ haloalkyl, C₃₋₆ cycloalkyl, —O(C₁₋₃ alkyl), and —O(C₁₋₃ haloalkyl);
   L³ and R⁶, together with the atoms to which they are attached, form 4- to 8-membered monocyclic heterocycloalkyl, 7- to 12-membered spirocyclic heterocycloalkyl, or 7- to 12-membered fused bicyclic heterocycloalkyl, each of which is optionally substituted with one, two, or three substituents selected from halogen, —OH, —CN, C₁₋₆ alkyl, C₃₋₆ cycloalkyl, —O(C₁₋₆ alkyl), and —O(C₃₋₆ cycloalkyl), wherein C₁₋₆ alkyl, C₃₋₆ cycloalkyl, —O(C₁₋₆ alkyl), and —O(C₃₋₆ cycloalkyl) are optionally substituted with one, two, or three substituents selected from halogen, —OH, —CN, C₁₋₃ alkyl, C₁₋₃ haloalkyl, C₃₋₆ cycloalkyl, —O(C₁₋₃ alkyl), and —O(C₁₋₃ haloalkyl), and wherein the 4- to 8-membered monocyclic heterocycloalkyl formed by L³ and R⁶ is not piperazine; or
   R² and R⁶, together with the atoms to which they are attached, form 3- to 8-membered monocyclic heterocycloalkyl optionally substituted with one, two, or three substituents selected from halogen, —OH, —CN, C₁₋₆ alkyl, C₃₋₆ cycloalkyl, —O(C₁₋₆ alkyl), and —O(C₃₋₆ cycloalkyl), wherein C₁₋₆ alkyl, C₃₋₆ cycloalkyl, —O(C₁₋₆ alkyl), and —O(C₃₋₆ cycloalkyl) are optionally substituted with one, two, or three substituents selected from halogen, —OH, —CN, C₁₋₃ alkyl, C₁₋₃ haloalkyl, C₃₋₆ cycloalkyl, —O(C₁₋₃ alkyl), and —O(C₁₋₃ haloalkyl), and wherein L³ is a bond;
R² is selected from halogen, —CN, C₁₋₆ alkyl, and C₃₋₆ cycloalkyl, or R² and R³, together with the carbon atom to which they are attached, form C₃₋₆ cycloalkyl, wherein C₁₋₆ alkyl and C₃₋₆ cycloalkyl are optionally substituted with one, two, or three substituents selected from halogen, —CN, C₁₋₆ alkyl, C₁₋₆ haloalkyl, C₃₋₆ cycloalkyl, —O(C₁₋₆ alkyl), and —O(C₁₋₆ haloalkyl);
R³ is selected from hydrogen, halogen, —CN, C₁₋₆ alkyl, and C₃₋₆ cycloalkyl, wherein C₁₋₆ alkyl and C₃₋₆ cycloalkyl are optionally substituted with one, two, or three substituents selected from halogen, —CN, C₁₋₆ alkyl, C₁₋₆ haloalkyl, C₃₋₆ cycloalkyl, —O(C₁₋₆ alkyl), and —O(C₁₋₆ haloalkyl);
R⁴, R⁵, and R⁶ are each independently selected from hydrogen, halogen, —CN, C₁₋₆ alkyl, and C₃₋₆ cycloalkyl, or R⁴ and R⁵, together with the carbon atom to which they are attached, form C₃₋₆ cycloalkyl, wherein C₁₋₆ alkyl and C₃₋₆ cycloalkyl are optionally substituted with one, two, or three substituents selected from halogen, —CN, C₁₋₆ alkyl, C₁₋₆ haloalkyl, C₃₋₆ cycloalkyl, —O(C₁₋₆ alkyl), and —O(C₁₋₆ haloalkyl);
R¹⁹ is independently selected at each occurrence from hydrogen, —CN, C₁₋₆ alkyl, C₃₋₆ carbocycle, 3- to 6-membered heterocycle, —OH, —O(C₁₋₆ alkyl), and —O(C₁₋₆ haloalkyl);

R²⁰ is independently selected at each occurrence from halogen, oxo, —CN, C₁₋₆ alkyl, C₂₋₆ alkenyl, C₂₋₆ alkynyl, 2- to 6-membered heteroalkyl, 2- to 6-membered heteroalkenyl, 2- to 6-membered heteroalkynyl, —C₀₋₆ alkyl-(C₃₋₁₂ carbocycle), -(2- to 6-membered heteroalkyl)-(C₃₋₁₂ carbocycle), —C₀₋₆ alkyl-(3- to 12-membered heterocycle), -(2- to 6-membered heteroalkyl)-(3- to 12-membered heterocycle), —OR²², —SR²², —N(R²²)(R²³), ═NR²², ═C(R²¹)₂, —C(O)OR²², —OC(O)N(R²²)(R²³), —N(R²²)C(O)N(R²²)(R²³), —N(R²²)C(O)OR²², —N(R²²)S(O)₂R²², —C(O)R²², —S(O)R²², —OC(O)R²², —C(O)N(R²²)(R²³), —C(O)C(O)N(R²²)(R²³), —N(R²²)C(O)R²², —S(O)₂R²², —S(O)(NR²²)R²², —S(O)₂N(R²²)(R²³)—, —S(═O)(═NR²²)N(R²²)(R²³), and —OCH₂C(O)OR²²; wherein two R²⁰ attached to the same or adjacent atoms optionally join to form C₃₋₁₂ carbocycle or 3- to 12-membered heterocycle; wherein C₁₋₆ alkyl, C₂₋₆ alkenyl, C₂₋₆ alkynyl, 2- to 6-membered heteroalkyl, 2- to 6-membered heteroalkenyl, 2- to 6-membered heteroalkynyl, —C₀₋₆ alkyl-(C₃₋₁₂ carbocycle), -(2- to 6-membered heteroalkyl)-(C₃₋₁₂ carbocycle), —C₀₋₆ alkyl-(3- to 12-membered heterocycle), (2- to 6-membered heteroalkyl)-(3- to 12-membered heterocycle), C₃₋₁₂ carbocycle, and 3- to 12-membered heterocycle are optionally substituted with one or more substituents independently selected from halogen, oxo, —CN, C₁₋₆ alkyl, C₁₋₆ haloalkyl, C₁₋₆ alkoxy, C₁₋₆ haloalkoxy, —OR²², —SR²², —N(R²²)(R²³), ═NR²², ═C(R²¹)₂, —C(O)OR²², —OC(O)N(R²²)(R²³), —N(R²²)C(O)N(R²²)(R²³), —N(R²²)C(O)OR²², —N(R²²)S(O)₂R²², —C(O)R²², —S(O)R²², —OC(O)R²², —C(O)N(R²²)(R²³), —C(O)C(O)N(R²²)(R²³), —N(R²²)C(O)R²², —S(O)₂R²², —S(O)(NR²²)R²², —S(O)₂N(R²²)(R²³), and —S(═O)(═NR²²)N(R²²)(R²³);
R²¹ is independently selected at each occurrence from hydrogen, halogen, C₁₋₆ alkyl, C₁₋₆ haloalkyl, —C₀₋₆ alkyl-(C₃₋₁₂ carbocycle), and —C₀₋₆ alkyl-(3- to 12-membered heterocycle), or two R²¹ are taken together with the carbon atom to which they are attached to form C₃₋₁₂ carbocycle or 3- to 12-membered heterocycle, each of which is optionally substituted with one, two, or three substituents independently selected from halogen, C₁₋₃ alkyl, C₁₋₃ haloalkyl, and —OH;
R²² is independently selected at each occurrence from hydrogen, C₁₋₆ alkyl, C₁₋₆ haloalkyl, —C₀₋₆ alkyl-(C₃₋₁₂ carbocycle), and —C₀₋₆ alkyl-(3- to 12-membered heterocycle); and
R²³ is independently selected at each occurrence from hydrogen and C₁₋₆ alkyl; or R²² and R²³ attached to the same nitrogen atom form 3- to 10 membered heterocycle.

In some embodiments, the modified protein of Formula (I) is a modified protein of Formula (I-a), (I-b), (I-c), or (I-d):

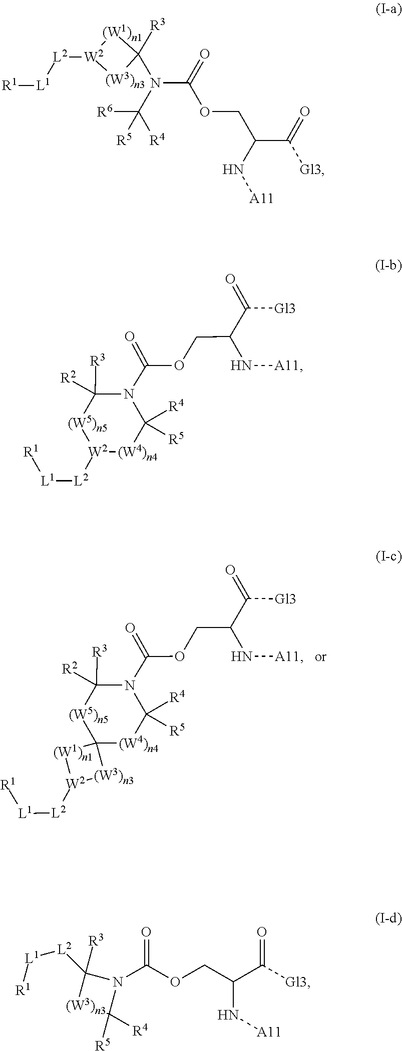

wherein:
W¹, W³, W⁴, and W⁵ are each independently selected from N(R¹⁰), C(R¹¹)₂, C(O), O, S(O), and S(O)₂;
W² is selected from N and C(R¹¹);
n1 and n3 are each independently selected from 0, 1, 2, 3, 4, and 5;
n4 and n5 are each independently selected from 0, 1, 2, 3, 4, and 5;
R¹⁰ is independently selected at each occurrence from hydrogen, C₁₋₆ alkyl, and C₃₋₆ cycloalkyl, wherein C₁₋₆ alkyl and C₃₋₆ cycloalkyl are optionally substituted with one, two, or three substituents selected from halogen, —OH, —CN, C₁₋₃ alkyl, C₁₋₃ haloalkyl, C₃₋₆ cycloalkyl, —O(C₁₋₃ alkyl), and —O(C₁₋₃ haloalkyl); and
R¹¹ is independently selected at each occurrence from hydrogen, halogen, —OH, —CN, C₁₋₆ alkyl, C₃₋₆ cycloalkyl, —O(C₁₋₆ alkyl), and —O(C₃₋₆ cycloalkyl), or two R¹¹ attached to the same carbon atom form C₃₋₆ cycloalkyl, wherein C₁₋₆ alkyl, C₃₋₆ cycloalkyl, —O(C₁₋₆ alkyl), and —O(C₃₋₆ cycloalkyl) are optionally substituted with one, two, or three substituents selected from halogen, —OH, —CN, C₁₋₃ alkyl, C₁₋₃ haloalkyl, C₃₋₆ cycloalkyl, —O(C₁₋₃ alkyl), and —O(C₁₋₃ haloalkyl).

In some embodiments, the modified Ras mutant protein described herein is formed by contacting a precursor compound with the serine residue of an unmodified Ras G12S mutant protein, wherein the precursor compound comprises a moiety susceptible to reacting with a nucleophilic serine residue corresponding to position 12 of SEQ ID No: 1. In some embodiments, the modified Ras protein is formed by contacting a compound disclosed herein, such as a compound of Formula (II), (II-a), (II-b), (II-c), or (II-d), with a serine residue of an unmodified Ras protein, such as an unmodified K-Ras G12S mutant protein. In some embodiments, the modified Ras protein is formed by contacting a precursor compound with a serine residue of an unmodified Ras G12S mutant protein, wherein the precursor compound comprises a staying group and a leaving group, and wherein said contacting results in release of the leaving group and formation of said modified protein. In some embodiments, the precursor compound is a compound disclosed herein, such as a compound of Formula (II), (II-a), (II-b), (II-c), or (II-d). In some embodiments, the leaving group is selected from

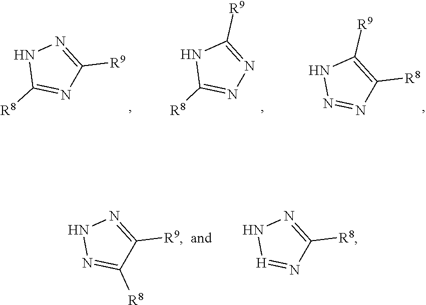

or a salt or tautomer thereof, wherein R⁸ and R⁹ are each independently selected from hydrogen, halogen, —CN, C₁₋₆ alkyl, and C₃₋₆ cycloalkyl, wherein C₁₋₆ alkyl and C₃₋₆ cycloalkyl are optionally substituted with one, two, or three substituents selected from halogen, —CN, C₁₋₆ alkyl, —O(C₁₋₆ alkyl), and —O(C₁₋₆ haloalkyl). In some embodiments, the leaving group is

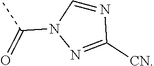

In some embodiments, a modified K-Ras G12S protein is formed by contacting a precursor compound with the serine residue of an unmodified Ras G12S mutant protein, wherein the precursor compound comprises a staying group and a leaving group, and wherein said leaving group separates from remainder of the precursor compound with an electron pair that previously formed a covalent bond between the leaving group and the remainder of the precursor compound after contacting the precursor compound with the unmodified Ras G12S mutant protein. In some embodiments, a modified K-Ras G12S protein is formed by contacting a precursor compound with the serine residue of an unmodified Ras G12S mutant protein, wherein the precursor compound comprises a staying group and a leaving group, and wherein said contacting results in release of the leaving group and formation of said modified protein. Release of the leaving group can be ascertained by a variety of methods known in the art, including without limitation mass spectroscopy. In particular, the molecular weight of the leaving group can be determined by the following formula:

$$L = [ U + P ] - [ M ]$$

wherein:
L is the molecular weight of the leaving group; U is the molecular weight of an unmodified Ras G12S mutant; P is the molecular weight of a subject precursor compound used to modify the unmodified Ras G12S mutant; M is the molecular weight of the modified Ras G12S mutant covalently bond to the precursor excluding the leaving group.

The molecular weight of the modified Ras G12S mutant can be ascertained by mass spectroscopy. In some embodiments, a subject compound, upon contacting an unmodified Ras G12S mutant protein (e.g., K-Ras G12S, H-Ras G12S, or N-Ras G12S), yields a leaving group of a molecular weight less than about 200 Da, such as less than about 190, 180, 170, 160, 150, 140, 130, 120, 110, 100, 90, 80, 70, 60, 50, 40, 30, 20 Da or less. One or more compound described herein, such as a compound of Table 1, yields, upon contacting K-Ras G12S, a leaving group of molecular weight less than 120 Da.

In some embodiments, a modified Ras mutant protein of the present disclosure exhibits a reduced Ras signaling output. A reduction of signaling output can be ascertained by a wide variety of methods known in the art. For example, phosphorylation of a substrate or a specific amino acid residue thereof can be detected and/or quantified using one or more techniques, such as kinase activity assays, phospho-specific antibodies, Western blot, enzyme-linked immunosorbent assays (ELISA), cell-based ELISA, intracellular flow cytometry, mass spectrometry, or multi-analyte profiling. The reduced Ras signaling output may be evidenced by one or more output selected from (i) an increase in steady state level of GDP-bound modified protein; (ii) a reduction in steady state level of GTP-bound modified protein; (iii) a reduction of phosphorylated AKTs473; (iv) a reduction of phosphorylated ERK T202/Y204; (v) a reduction of phosphorylated S6 S235/236; (vi) a reduction of cell growth of a tumor cell expressing a Ras G12S mutant protein; and (vii) a reduction in Ras interaction with a Ras-pathway signaling protein. In some embodiments, a reduction is evidenced by 2, 3, 4 or more of items (i)-(vii). In some embodiments, the reduction in Ras signaling output can be evidenced by any one of (i)-(vii) as compared to a control unmodified corresponding Ras protein that is not covalently bonded to a compound disclosed herein. For example, a control Ras protein, as described herein, can be a Ras protein (e.g., wildtype or mutated) that is not complexed with a compound of the present disclosure. The increase in item (i) or reduction in items (ii) through (vii) can be at least about 0.1-fold, 0.2-fold, 0.3-fold, 0.4-fold, 0.5-fold, 0.6-fold, 0.7-fold, 0.8-fold, 0.9-fold, 1-fold, 2-fold, 3-fold, 4-fold, 5-fold, 6- fold, 7-fold, 8-fold, 9-fold, 10-fold, 20-fold, 30-fold, 40-fold, 50-fold, 60-fold, 70-fold, 80-fold, 90-fold, 100-fold, 200-fold, 300-fold, 400-fold, 500-fold, 600-fold, 700-fold, 800-fold, 900-fold, 1000-fold, 2000-fold, 3000-fold, 4000-fold, 5000-fold, or more as compared to the control Ras protein. In some embodiments, a reduction in Ras interaction with a Ras-pathway signaling protein is established by observing a reduced interaction with SOS (including SOS1 and SOS2), RAF, SHC, SHP (including SHP1 and SHP2), MEK, MAPK, ERK, GRB, RASA1, and/or GNAQ.

Signaling output measured in terms of IC50 values can be obtained and a ratio of IC50 against one mutant relative to another mutant can be calculated. For instance, a selective reduction of K-Ras G12S signaling output can be evidenced by a ratio greater than one. In particular, a selective reduction of K-Ras G12S signaling relative to K-Ras G12D signaling is evidenced when the ratio of IC50 (against K-Ras G12D) to IC50 (against K-Ras G12S) is greater than 1. One or more compound disclosed herein, such as a compound of Table 1, exhibits selective inhibition of K-Ras G12S relative to K-Ras G12D by at least 1-fold, and in some instances greater than 2-, 3-, 4- or 5-fold. In some embodiments, a compound of the present disclosure exhibits an IC50 against K-Ras G12S less than 500 nM, such as less than 100 nM, 50 nM, 10 nM or even less. Applying one or more methods exemplified herein, a subject compound may exhibit selective labeling of a Ras G12S mutant relative to a Ras G12D mutant or wildtype protein. Exemplary compounds, such as a compound of Table 1, may covalently label K-Ras G12S mutant by at least 1%, 10%, 20%, 50% or more with no detectable labeling observed for K-Ras G12D or K-Ras wildtype when tested under the same or comparable or conditions.

In some embodiments, the modified Ras protein comprises an amino acid sequence in SEQ ID No. 4, or a fragment thereof comprising a serine residue corresponding to position 12 of SEQ ID No. 1. In some embodiments, the modified Ras protein comprises an amino acid sequence of SEQ ID No. 4. In some embodiments, the modified protein comprises an amino acid sequence of SEQ ID No. 1, or a fragment thereof that comprises a serine residue corresponding to position 12 of SEQ ID No. 1, wherein the precursor compound selectively labels the serine residue as compared to (i) an aspartate residue of a K-Ras G12D mutant protein, said aspartate corresponding to position 12 of SEQ ID No. 2; (ii) a valine residue of a K-Ras G12V mutant protein, said valine corresponding to position 12 of SEQ ID No. 3; and/or (iii) a glycine residue of a K-Ras wildtype protein, said glycine corresponding to position 12 of SEQ ID No. 1. In some embodiments, the precursor compound selectively labels the serine residue in SEQ ID No. 4 by at least 2-fold, 3-fold, 4-fold, or 5-fold when assayed under comparable conditions. In some embodiments, the precursor compound selectively labels the serine residue by more than 5-fold when assayed under comparable conditions. It will be understood that when a compound of the present disclosure selectively labels the serine residue of a K-Ras G12S protein compared to another K-Ras protein(s) (e.g., WT, G12D, G12V), the compound labels the K-Ras G12S protein with greater speed or to a greater degree, or by any other quantifiable measurement, compared to the other K-Ras protein (e.g., WT, G12D, G12V), under similar or identical reaction conditions for the proteins being compared. In some embodiments, the greater labeling of K-Ras G12S can be 0.1-fold, 0.2-fold, 0.3-fold, 0.4-fold, 0.5-fold, 0.6-fold, 0.7-fold, 0.8-fold, 0.9-fold, 1-fold, 2-fold, 3-fold, 4-fold, 5-fold, 6-fold, 7-fold, 8-fold, 9-fold, 10-fold, 20-fold, 30-fold, 40-fold, 50-fold, 60-fold, 70-fold, 80-fold, 90-fold, 100-fold, 200-fold, 300-fold, 400-fold, 500-fold, 600-fold, 700-fold, 800-fold, 900-fold, 1000-fold, 2000-fold, 3000-fold, 4000-fold, 5000-fold, or more as compared to another K-Ras protein (e.g., WT, G12D, G12V). In some embodiments, the speed of labeling of K-Ras G12S can be 0.1-fold, 0.2-fold, 0.3-fold, 0.4-fold, 0.5-fold, 0.6-fold, 0.7-fold, 0.8-fold, 0.9-fold, 1-fold, 2-fold, 3-fold, 4-fold, 5-fold, 6-fold, 7-fold, 8-fold, 9-fold, 10-fold, 20-fold, 30-fold, 40- fold, 50-fold, 60-fold, 70-fold, 80-fold, 90-fold, 100-fold, 200-fold, 300-fold, 400-fold, 500-fold, 600-fold, 700-fold, 800-fold, 900-fold, 1000-fold, 2000-fold, 3000-fold, 4000-fold, 5000-fold or more, at a selected time point during the course of labeling, as compared to another K-Ras protein (e.g., WT, G12D, G12V).

In some embodiments, a compound of the present disclosure selectively labels the serine residue as compared to (i) an aspartate residue of a K-Ras G12D mutant protein, said aspartate corresponding to residue 12 of SEQ ID NO: 2, and/or (ii) a valine residue of a K-Ras G12V mutant protein, said valine corresponding to residue 12 of SEQ ID NO: 3, by at least 0.1, 0.2, 0.3, 0.4, 0.5, 1, 2, 3, 4, 5, 10 fold or more, when assayed under comparable conditions. In some embodiments, a compound of the present disclosure selectively labels (e.g., via covalent binding) the serine residue of an unmodified Ras G12S protein corresponding to position 12 of SEQ ID No: 4 in vitro. In some embodiments, a compound of the present disclosure selectively labels (e.g., via covalent binding) the serine residue of an unmodified Ras G12S protein corresponding to position 12 of SEQ ID No: 4 in vivo.

The compounds of Formula (II) disclosed herein-including the compounds of Formula (II-a), (II-b), (II-c), and (II-d) or a pharmaceutically acceptable salt or solvate thereof, are K-Ras inhibitors and have a wide range of applications in therapeutics, diagnostics, and other biomedical research. In some embodiments, a compound disclosed herein covalently modifies a Ras protein, such as a K-Ras G12S protein. In some embodiments, a Ras protein, such as a K-Ras G12S protein, is contacted with a compound disclosed herein to form a modified Ras protein.

In certain aspects, the present disclosure provides a compound of Formula (II):

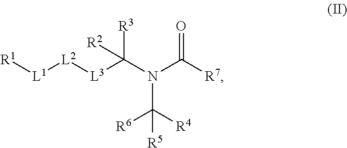

or a pharmaceutically acceptable salt or solvate thereof, wherein:
R¹ is selected from C₃₋₁₂ carbocycle and 3- to 12-membered heterocycle, each of which is optionally substituted with one or more R²⁰;
L¹ is 5- to 20-membered heterocycle optionally substituted with one or more R²⁰;
L² is selected from a bond, C₁₋₄ alkylene, 2- to 4-membered heteroalkylene, —O—, —N(R¹⁹)—, —C(O)—, —S—, —S(O)₂—, —S(O)—, —P(O)R¹⁹—, —N(R¹⁹)S(O)₂—, —N(R¹⁹)S(O)—, —N(R¹⁹)P(O)R¹⁹—, —S(O)₂N(R¹⁹)—, —S(O)N(R¹⁹)—, —P(O)R¹⁹N(R¹⁹)—, —OS(O)₂—, —OS(O)—, —OP(O)R¹⁹—, —S(O)₂O—, —S(O)O—, and —P(O)R¹⁹O—, wherein C₁₋₄ alkylene and 2- to 4-membered heteroalkylene are optionally substituted with one, two, or three R²⁰;
   L³ and R², together with the atoms to which they are attached, form 3- to 8-membered monocyclic heterocycloalkyl, C₃₋₈ monocyclic cycloalkyl, 7- to 12-membered spirocyclic heterocycloalkyl, or 7- to 12-membered fused bicyclic heterocycloalkyl, each of which is optionally substituted with one, two, or three R²⁰;
   L³ and R⁶, together with the atoms to which they are attached, form 4- to 8-membered monocyclic heterocycloalkyl, 7- to 12-membered spirocyclic heterocycloalkyl, or 7- to 12-membered fused bicyclic heterocycloalkyl, each of which is optionally substituted with one, two, or three R²⁰, wherein the 4- to 8-membered monocyclic heterocycloalkyl formed by L³ and R⁶ is not piperazine; or
   R² and R⁶, together with the atoms to which they are attached, form 3- to 8-membered monocyclic heterocycloalkyl optionally substituted with one, two, or three R²⁰, wherein L³ is a bond;
R² is selected from R²⁰, or R² and R³, together with the carbon atom to which they are attached, form C₃₋₆ cycloalkyl optionally substituted with one, two, or three R²⁰;
R³ is selected from hydrogen and R²⁰;
R⁴, R⁵, and R⁶ are each independently selected from hydrogen and R²⁰, or R⁴ and R⁵, together with the carbon atom to which they are attached, form C₃₋₆ cycloalkyl optionally substituted with one, two, or three R²⁰;
R⁷ is selected from

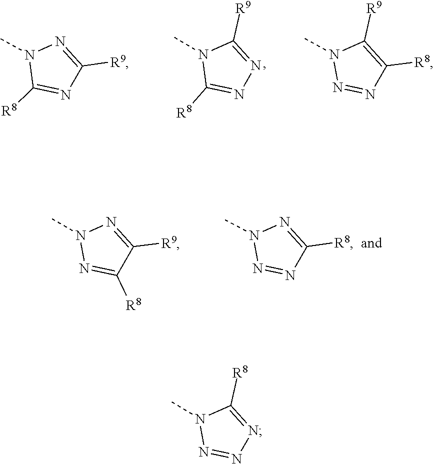

R⁸ and R⁹ are each independently selected from hydrogen and R²⁰;
R¹⁹ is independently selected at each occurrence from hydrogen, —CN, C₁₋₆ alkyl, C₂₋₆ alkenyl, C₂₋₆ alkynyl, —C₀₋₆ alkyl-(C₃₋₁₂ carbocycle), —C₀₋₆ alkyl-(3- to 12-membered heterocycle), —OH, and —O(C₁₋₆ alkyl), wherein C₁₋₆ alkyl, C₂₋₆ alkenyl, C₂₋₆ alkynyl, —C₀₋₆ alkyl-(C₃₋₁₂ carbocycle), —C₀₋₆ alkyl-(3- to 12-membered heterocycle), and —O(C₁₋₆ alkyl) are optionally substituted with one, two, or three R²⁰;

R²⁰ is independently selected at each occurrence from halogen, oxo, —CN, C₁₋₆ alkyl, C₂₋₆ alkenyl, C₂₋₆ alkynyl, 2- to 6-membered heteroalkyl, 2- to 6-membered heteroalkenyl, 2- to 6-membered heteroalkynyl, —C₀₋₆ alkyl-(C₃₋₁₂ carbocycle), -(2- to 6-membered heteroalkyl)-(C₃₋₁₂ carbocycle), —C₀₋₆ alkyl-(3- to 12-membered heterocycle), -(2- to 6-membered heteroalkyl)-(3- to 12-membered heterocycle), —OR²², —SR²², —N(R²²)(R²³), ═NR²², —C(R²¹)₂, —C(O)OR²², —OC(O)N(R²²)(R²³), —N(R²²)C(O)N(R²²)(R²³), —N(R²²)C(O)OR²², —N(R²²)S(O)₂R²², —C(O)R²², —S(O)R²², —OC(O)R²², —C(O)N(R²²)(R²³), —C(O)C(O)N(R²²)(R²³), —N(R²²)C(O)R²², —S(O)₂R²², —S(O)(NR²²)R²², —S(O)₂N(R²²)(R²³)—, —S(—O)(═NR²²)N(R²²)(R²³), and —OCH₂C(O)OR²²; wherein two R²⁰ attached to the same or adjacent atoms optionally join to form C₃₋₁₂ carbocycle or 3- to 12-membered heterocycle; wherein C₁₋₆ alkyl, C₂₋₆ alkenyl, C₂₋₆ alkynyl, 2- to 6-membered heteroalkyl, 2- to 6-membered heteroalkenyl, 2- to 6-membered heteroalkynyl, —C₀₋₆ alkyl-(C₃₋₁₂ carbocycle), -(2- to 6-membered heteroalkyl)-(C₃₋₁₂ carbocycle), —C₀₋₆ alkyl-(3- to 12-membered heterocycle), -(2- to 6-membered heteroalkyl)-(3- to 12-membered heterocycle), C₃₋₁₂ carbocycle, and 3- to 12-membered heterocycle are optionally substituted with one or more substituents independently selected from halogen, oxo, —CN, C₁₋₆ alkyl, C₁₋₆ haloalkyl, C₁₋₆ alkoxy, C₁₋₆ haloalkoxy, —OR²², —SR²², —N(R²²)(R²³), ═NR²², ═C(R²¹)₂, —C(O)OR²², —OC(O)N(R²²)(R²³), —N(R²²)C(O)N(R²²)(R²³), —N(R²²)C(O)OR²², —N(R²²)S(O)₂R²², —C(O)R²², —S(O)R²², —OC(O)R²², —C(O)N(R²²)(R²³), —C(O)C(O)N(R²²)(R²³), —N(R²²)C(O)R²², —S(O)₂R²², —S(O)(NR²²)R²², —S(O)₂N(R²²)(R²³), and —S(═O)(═NR²²)N(R²²)(R²³);
R²¹ is independently selected at each occurrence from hydrogen, halogen, C₁₋₆ alkyl, C₂₋₆ alkenyl, C₂₋₆ alkynyl, —C₀₋₆ alkyl-(C₃₋₁₂ carbocycle), and —C₀₋₆ alkyl-(3- to 12-membered heterocycle), or two R²¹ are taken together with the carbon atom to which they are attached to form C₃₋₁₂ carbocycle or 3- to 12-membered heterocycle, wherein C₁₋₆ alkyl, C₂₋₆ alkenyl, C₂₋₆ alkynyl, —C₀₋₆ alkyl-(C₃₋₁₂ carbocycle), —C₀₋₆ alkyl-(3- to 12-membered heterocycle), C₃₋₁₂ carbocycle, and 3- to 12-membered heterocycle are optionally substituted with one, two, or three R²⁰;
R²² is independently selected at each occurrence from hydrogen, C₁₋₆ alkyl, C₂₋₆ alkenyl, C₂₋₆ alkynyl, —C₀₋₆ alkyl-(C₃₋₁₂ carbocycle), and —C₀₋₆ alkyl-(3- to 12-membered heterocycle), wherein C₁₋₆ alkyl, C₂₋₆ alkenyl, C₂₋₆ alkynyl, —C₀₋₆ alkyl-(C₃₋₁₂ carbocycle), and —C₀₋₆ alkyl-(3- to 12-membered heterocycle) are optionally substituted with one, two, or three R²⁰; and
R²³ is independently selected at each occurrence from hydrogen, C₁₋₆ alkyl, and C₁₋₆ haloalkyl; or R²² and R²³ attached to the same nitrogen atom form 3- to 10-membered heterocycle optionally substituted with one, two, or three R²⁰.

In certain aspects, the present disclosure provides a compound of Formula (II):

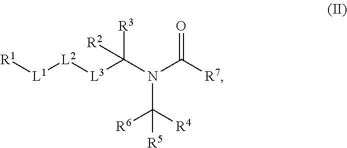

or a pharmaceutically acceptable salt or solvate thereof, wherein:
R¹ is selected from C₃₋₁₂ carbocycle and 3- to 12-membered heterocycle, each of which is optionally substituted with one or more R²⁰;
L¹ is 5- to 20-membered heterocycle optionally substituted with one or more R²⁰;
L² is selected from a bond, C₁₋₄ alkylene, 2- to 4-membered heteroalkylene, —O—, —N(R¹⁹)—, —C(O)—, —S—, —S(O)₂—, —S(O)—, —P(O)R¹⁹—, —N(R¹⁹)S(O)₂—, —N(R¹⁹)S(O)—, —N(R¹⁹)P(O)R¹⁹—, —S(O)₂N(R¹⁹)—, —S(O)N(R¹⁹)—, —P(O)R¹⁹N(R¹⁹)—, —OS(O)₂—, —OS(O)—, —OP(O)R¹⁹—, —S(O)₂O—, —S(O)O—, and —P(O)R¹⁹O—, wherein C₁₋₄ alkylene and 2- to 4-membered heteroalkylene are optionally substituted with one, two, or three R²⁰;
   L³ and R², together with the atoms to which they are attached, form 3- to 8-membered monocyclic heterocycloalkyl, C₃₋₈ monocyclic cycloalkyl, 7- to 12-membered spirocyclic heterocycloalkyl, or 7- to 12-membered fused bicyclic heterocycloalkyl, each of which is optionally substituted with one, two, or three substituents selected from halogen, —OH, —CN, C₁₋₆ alkyl, C₃₋₆ cycloalkyl, —O(C₁₋₆ alkyl), and —O(C₃₋₆ cycloalkyl), wherein C₁₋₆ alkyl, C₃₋₆ cycloalkyl, —O(C₁₋₆ alkyl), and —O(C₃₋₆ cycloalkyl) are optionally substituted with one, two, or three substituents selected from halogen, —OH, —CN, C₁₋₃ alkyl, C₁₋₃ haloalkyl, C₃₋₆ cycloalkyl, —O(C₁₋₃ alkyl), and —O(C₁₋₃ haloalkyl);
   L³ and R⁶, together with the atoms to which they are attached, form 4- to 8-membered monocyclic heterocycloalkyl, 7- to 12-membered spirocyclic heterocycloalkyl, or 7- to 12-membered fused bicyclic heterocycloalkyl, each of which is optionally substituted with one, two, or three substituents selected from halogen, —OH, —CN, C₁₋₆ alkyl, C₃₋₆ cycloalkyl, —O(C₁₋₆ alkyl), and —O(C₃₋₆ cycloalkyl), wherein C₁₋₆ alkyl, C₃₋₆ cycloalkyl, —O(C₁₋₆ alkyl), and —O(C₃₋₆ cycloalkyl) are optionally substituted with one, two, or three substituents selected from halogen, —OH, —CN, C₁₋₃ alkyl, C₁₋₃ haloalkyl, C₃₋₆ cycloalkyl, —O(C₁₋₃ alkyl), and —O(C₁₋₃ haloalkyl), and wherein the 4- to 8-membered monocyclic heterocycloalkyl formed by L³ and R⁶ is not piperazine; or
   R² and R⁶, together with the atoms to which they are attached, form 3- to 8-membered monocyclic heterocycloalkyl optionally substituted with one, two, or three substituents selected from halogen, —OH, —CN, C₁₋₆ alkyl, C₃₋₆ cycloalkyl, —O(C₁₋₆ alkyl), and —O(C₃₋₆ cycloalkyl), wherein C₁₋₆ alkyl, C₃₋₆ cycloalkyl, —O(C₁₋₆ alkyl), and —O(C₃₋₆ cycloalkyl) are optionally substituted with one, two, or three substituents selected from halogen, —OH, —CN, C₁₋₃ alkyl, C₁₋₃ haloalkyl, C₃₋₆ cycloalkyl, —O(C₁₋₃ alkyl), and —O(C₁₋₃ haloalkyl), and wherein L³ is a bond;
R² is selected from halogen, —CN, C₁₋₆ alkyl, and C₃₋₆ cycloalkyl, or R² and R³, together with the carbon atom to which they are attached, form C₃₋₆ cycloalkyl, wherein C₁₋₆ alkyl and C₃₋₆ cycloalkyl are optionally substituted with one, two, or three substituents selected from halogen, —CN, C₁₋₆ alkyl, C₁₋₆ haloalkyl, C₃₋₆ cycloalkyl, —O(C₁₋₆ alkyl), and —O(C₁₋₆ haloalkyl);
R³ is selected from hydrogen, halogen, —CN, C₁₋₆ alkyl, and C₃₋₆ cycloalkyl, wherein C₁₋₆ alkyl and C₃₋₆ cycloalkyl are optionally substituted with one, two, or three substituents selected from halogen, —CN, C₁₋₆ alkyl, C₁₋₆ haloalkyl, C₃₋₆ cycloalkyl, —O(C₁₋₆ alkyl), and —O(C₁₋₆ haloalkyl);
R⁴, R⁵, and R⁶ are each independently selected from hydrogen, halogen, —CN, C₁₋₆ alkyl, and C₃₋₆ cycloalkyl, or R⁴ and R⁵, together with the carbon atom to which they are attached, form C₃₋₆ cycloalkyl, wherein C₁₋₆ alkyl and C₃₋₆ cycloalkyl are optionally substituted with one, two, or three substituents selected from halogen, —CN, C₁₋₆ alkyl, C₁₋₆ haloalkyl, C₃₋₆ cycloalkyl, —O(C₁₋₆ alkyl), and —O(C₁₋₆ haloalkyl);
R⁷ is selected from

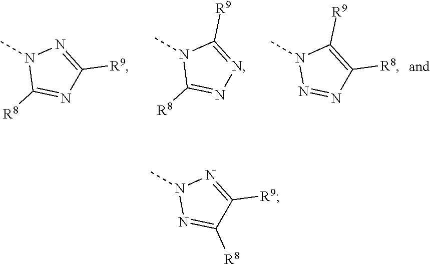

R⁸ and R⁹ are each independently selected from hydrogen, halogen, —CN, C₁₋₆ alkyl, and C₃₋₆ cycloalkyl, wherein C₁₋₆ alkyl and C₃₋₆ cycloalkyl are optionally substituted with one, two, or three substituents selected from halogen, —CN, C₁₋₆ alkyl, —O(C₁₋₆ alkyl), and —O(C₁₋₆ haloalkyl);
R¹⁹ is independently selected at each occurrence from hydrogen, —CN, C₁₋₆ alkyl, C₃₋₆ carbocycle, 3- to 6-membered heterocycle, —OH, —O(C₁₋₆ alkyl), and —O(C₁₋₆ haloalkyl);
R²⁰ is independently selected at each occurrence from halogen, oxo, —CN, C₁₋₆ alkyl, C₂₋₆ alkenyl, C₂₋₆ alkynyl, 2- to 6-membered heteroalkyl, 2- to 6-membered heteroalkenyl, 2- to 6-membered heteroalkynyl, —C₀₋₆ alkyl-(C₃₋₁₂ carbocycle), -(2- to 6-membered heteroalkyl)-(C₃₋₁₂ carbocycle), —C₀₋₆ alkyl-(3- to 12-membered heterocycle), -(2- to 6-membered heteroalkyl)-(3- to 12-membered heterocycle), —OR²², —SR²², —N(R²²)(R²³), ═NR²², —C(R²¹)₂, —C(O)OR²², —OC(O)N(R²²)(R²³), —N(R²²)C(O)N(R²²)(R²³), —N(R²²)C(O)OR²², —N(R²²)S(O)₂R²², —C(O)R²², —S(O)R²², —OC(O)R²², —C(O)N(R²²)(R²³), —C(O)C(O)N(R²²)(R²³), —N(R²²)C(O)R²², —S(O)₂R²², —S(O)(NR²²)R²², —S(O)₂N(R²²)(R²³)—, —S(═O)(—NR²²)N(R²²)(R²³), and —OCH₂C(O)OR²²; wherein two R²⁰ attached to the same or adjacent atoms optionally join to form C₃₋₁₂ carbocycle or 3- to 12-membered heterocycle; wherein C₁₋₆ alkyl, C₂₋₆ alkenyl, C₂₋₆ alkynyl, 2- to 6-membered heteroalkyl, 2- to 6-membered heteroalkenyl, 2- to 6-membered heteroalkynyl, —C₀₋₆ alkyl-(C₃₋₁₂ carbocycle), -(2- to 6-membered heteroalkyl)-(C₃₋₁₂ carbocycle), —C₀₋₆ alkyl-(3- to 12-membered heterocycle), -(2- to 6-membered heteroalkyl)-(3- to 12-membered heterocycle), C₃₋₁₂ carbocycle, and 3- to 12-membered heterocycle are optionally substituted with one or more substituents independently selected from halogen, oxo, —CN, C₁₋₆ alkyl, C₁₋₆ haloalkyl, C₁₋₆ alkoxy, C₁₋₆ haloalkoxy, —OR²², —SR²², —N(R²²)(R²³), —NR²², ═C(R²¹)₂, —C(O)OR²², —OC(O)N(R²²)(R²³), —N(R²²)C(O)N(R²²)(R²³), —N(R²²)C(O)OR²², —N(R²²)S(O)₂R²², —C(O)R²², —S(O)R²², —OC(O)R²², —C(O)N(R²²)(R²³), —C(O)C(O)N(R²²)(R²³), —N(R²²)C(O)R²², —S(O)₂R²², —S(O)(NR²²)R²², —S(O)₂N(R²²)(R²³), and —S(═O)(═NR²²)N(R²²)(R²³);
R²¹ is independently selected at each occurrence from hydrogen, halogen, C₁₋₆ alkyl, C₁₋₆ haloalkyl, —C₀₋₆ alkyl-(C₃₋₁₂ carbocycle), and —C₀₋₆ alkyl-(3- to 12-membered heterocycle), or two R²¹ are taken together with the carbon atom to which they are attached to form C₃₋₁₂ carbocycle or 3- to 12-membered heterocycle, each of which is optionally substituted with one, two, or three substituents independently selected from halogen, C₁₋₃ alkyl, C₁₋₃ haloalkyl, and —OH;
R²² is independently selected at each occurrence from hydrogen, C₁₋₆ alkyl, C₁₋₆ haloalkyl, —C₀₋₆ alkyl-(C₃₋₁₂ carbocycle), and —C₀₋₆ alkyl-(3- to 12-membered heterocycle); and
R²³ is independently selected at each occurrence from hydrogen and C₁₋₆ alkyl; or R²² and R²³ attached to the same nitrogen atom form 3- to 10 membered heterocycle.

In some embodiments, for a compound of Formula (II) or a modified protein of Formula (I′) or (I), L³ and R², together with the atoms to which they are attached, form 3- to 8-membered monocyclic heterocycloalkyl, C₃₋₈ monocyclic cycloalkyl, 7- to 12-membered spirocyclic heterocycloalkyl, or 7- to 12-membered fused bicyclic heterocycloalkyl, each of which is optionally substituted with one, two, or three R²⁰. In some embodiments, for a compound of Formula (II) or a modified protein of Formula (I′) or (I), L³ and R², together with the atoms to which they are attached, form 3- to 8-membered monocyclic heterocycloalkyl, 7- to 12-membered spirocyclic heterocycloalkyl, or 7- to 12-membered fused bicyclic heterocycloalkyl, each of which is optionally substituted with one, two, or three R²⁰. In some embodiments, L³ and R², together with the atoms to which they are attached, form 3- to 8-membered monocyclic heterocycloalkyl optionally substituted with one, two, or three R²⁰. In some embodiments, L³ and R⁶, together with the atoms to which they are attached, form 4- to 8-membered monocyclic heterocycloalkyl, 7- to 12-membered spirocyclic heterocycloalkyl, or 7- to 12-membered fused bicyclic heterocycloalkyl, each of which is optionally substituted with one, two, or three R²⁰, wherein the 4- to 8-membered monocyclic heterocycloalkyl formed by L³ and R⁶ is not piperazine. In some embodiments, R² and R⁶, together with the atoms to which they are attached, form 3- to 8-membered monocyclic heterocycloalkyl optionally substituted with one, two, or three R²⁰, wherein L³ is a bond. In some embodiments, the ring formed by L³ and R², L³ and R⁶, or R² and R⁶ is substituted with at least one R²⁰, such as one R²⁰, two R²⁰, three R²⁰, four R²⁰, or five R²⁰.

In some embodiments, for a compound of Formula (II) or a modified protein of Formula (I′) or (I), L³ and R², together with the atoms to which they are attached, form 3- to 8-membered monocyclic heterocycloalkyl, C₃₋₈ monocyclic cycloalkyl, 7- to 12-membered spirocyclic heterocycloalkyl, or 7- to 12-membered fused bicyclic heterocycloalkyl, each of which is optionally substituted with one, two, or three substituents selected from halogen, —OH, —CN, C₁₋₆ alkyl, C₃₋₆ cycloalkyl, —O(C₁₋₆ alkyl), and —O(C₃₋₆ cycloalkyl), wherein C₁₋₆ alkyl, C₃₋₆ cycloalkyl, —O(C₁₋₆ alkyl), and —O(C₃₋₆ cycloalkyl) are optionally substituted with one, two, or three substituents selected from halogen, —OH, —CN, C₁₋₃ alkyl, C₁₋₃ haloalkyl, C₃₋₆ cycloalkyl, —O(C₁₋₃ alkyl), and —O(C₁₋₃ haloalkyl). In some embodiments, for a compound of Formula (II) or a modified protein of Formula (I′) or (I), L³ and R², together with the atoms to which they are attached, form 3- to 8-membered monocyclic heterocycloalkyl, 7- to 12-membered spirocyclic heterocycloalkyl, or 7- to 12-membered fused bicyclic heterocycloalkyl, each of which is optionally substituted with one, two, or three substituents selected from halogen, —OH, —CN, C₁₋₆ alkyl, C₃₋₆ cycloalkyl, —O(C₁₋₆ alkyl), and —O(C₃₋₆ cycloalkyl), wherein C₁₋₆ alkyl, C₃₋₆ cycloalkyl, —O(C₁₋₆ alkyl), and —O(C₃₋₆ cycloalkyl) are optionally substituted with one, two, or three substituents selected from halogen, —OH, —CN, C₁₋₃ alkyl, C₁₋₃ haloalkyl, C₃₋₆ cycloalkyl, —O(C₁₋₃ alkyl), and —O(C₁₋₃ haloalkyl). In some embodiments, L³ and R², together with the atoms to which they are attached, form 3- to 8-membered monocyclic heterocycloalkyl optionally substituted with one, two, or three substituents selected from halogen, —OH, —CN, C₁₋₆ alkyl, C₃₋₆ cycloalkyl, —O(C₁₋₆ alkyl), and —O(C₃₋₆ cycloalkyl), wherein C₁₋₆ alkyl, C₃₋₆ cycloalkyl, —O(C₁₋₆ alkyl), and —O(C₃₋₆ cycloalkyl) are optionally substituted with one, two, or three substituents selected from halogen, —OH, —CN, C₁₋₃ alkyl, C₁₋₃ haloalkyl, C₃₋₆ cycloalkyl, —O(C₁₋₃ alkyl), and —O(C₁₋₃ haloalkyl). In some embodiments, L³ and R⁶, together with the atoms to which they are attached, form 4- to 8-membered monocyclic heterocycloalkyl, 7- to 12-membered spirocyclic heterocycloalkyl, or 7- to 12-membered fused bicyclic heterocycloalkyl, each of which is optionally substituted with one, two, or three substituents selected from halogen, —OH, —CN, C₁₋₆ alkyl, C₃₋₆ cycloalkyl, —O(C₁₋₆ alkyl), and —O(C₃₋₆ cycloalkyl), wherein C₁₋₆ alkyl, C₃₋₆ cycloalkyl, —O(C₁₋₆ alkyl), and —O(C₃₋₆ cycloalkyl) are optionally substituted with one, two, or three substituents selected from halogen, —OH, —CN, C₁₋₃ alkyl, C₁₋₃ haloalkyl, C₃₋₆ cycloalkyl, —O(C₁₋₃ alkyl), and —O(C₁₋₃ haloalkyl), and wherein the 4- to 8-membered monocyclic heterocycloalkyl formed by L³ and R⁶ is not piperazine. In some embodiments, R² and R⁶, together with the atoms to which they are attached, form 3- to 8-membered monocyclic heterocycloalkyl optionally substituted with one, two, or three substituents selected from halogen, —OH, —CN, C₁₋₆ alkyl, C₃₋₆ cycloalkyl, —O(C₁₋₆ alkyl), and —O(C₃₋₆ cycloalkyl), wherein C₁₋₆ alkyl, C₃₋₆ cycloalkyl, —O(C₁₋₆ alkyl), and —O(C₃₋₆ cycloalkyl) are optionally substituted with one, two, or three substituents selected from halogen, —OH, —CN, C₁₋₃ alkyl, C₁₋₃ haloalkyl, C₃₋₆ cycloalkyl, —O(C₁₋₃ alkyl), and —O(C₁₋₃ haloalkyl), and wherein L³ is a bond. In some embodiments, the ring formed by L³ and R², L³ and R⁶, or R² and R⁶ is unsubstituted. In some embodiments, the ring formed by L³ and R², L³ and R⁶, or R² and R⁶ is substituted with one substituent selected from halogen, —OH, —CN, C₁₋₆ alkyl, C₃₋₆ cycloalkyl, —O(C₁₋₆ alkyl), and —O(C₃₋₆ cycloalkyl), wherein C₁₋₆ alkyl, C₃₋₆ cycloalkyl, —O(C₁₋₆ alkyl), and —O(C₃₋₆ cycloalkyl) are optionally substituted with one, two, or three substituents selected from halogen, —OH, —CN, C₁₋₃ alkyl, C₁₋₃ haloalkyl, C₃₋₆ cycloalkyl, —O(C₁₋₃ alkyl), and —O(C₁₋₃ haloalkyl). In some embodiments, the ring formed by L³ and R², L³ and R⁶, or R² and R⁶ is substituted with two substituents independently selected from halogen, —OH, —CN, C₁₋₆ alkyl, C₃₋₆ cycloalkyl, —O(C₁₋₆ alkyl), and —O(C₃₋₆ cycloalkyl), wherein C₁₋₆ alkyl, C₃₋₆ cycloalkyl, —O(C₁₋₆ alkyl), and —O(C₃₋₆ cycloalkyl) are optionally substituted with one, two, or three substituents selected from halogen, —OH, —CN, C₁₋₃ alkyl, C₁₋₃ haloalkyl, C₃₋₆ cycloalkyl, —O(C₁₋₃ alkyl), and —O(C₁₋₃ haloalkyl). In some embodiments, the ring formed by L³ and R², L³ and R⁶, or R² and R⁶ is substituted with three substituents independently selected from halogen, —OH, —CN, C₁₋₆ alkyl, C₃₋₆ cycloalkyl, —O(C₁₋₆ alkyl), and —O(C₃₋₆ cycloalkyl), wherein C₁₋₆ alkyl, C₃₋₆ cycloalkyl, —O(C₁₋₆ alkyl), and —O(C₃₋₆ cycloalkyl) are optionally substituted with one, two, or three substituents selected from halogen, —OH, —CN, C₁₋₃ alkyl, C₁₋₃ haloalkyl, C₃₋₆ cycloalkyl, —O(C₁₋₃ alkyl), and —O(C₁₋₃ haloalkyl). In some embodiments, the ring formed by L³ and R², L³ and R⁶, or R² and R⁶ is substituted with one, two, or three substituents independently selected from halogen, —OH, —CN, C₁₋₆ alkyl, C₃₋₆ cycloalkyl, —O(C₁₋₆ alkyl), and —O(C₃₋₆ cycloalkyl), wherein C₁₋₆ alkyl, C₃₋₆ cycloalkyl, —O(C₁₋₆ alkyl), and —O(C₃₋₆ cycloalkyl) are optionally substituted with one, two, or three substituents selected from R²⁰. In some embodiments, the ring formed by L³ and R², L³ and R⁶, or R² and R⁶ is substituted with one substituent selected from halogen, —(C₀₋₆ alkyl)-CN, C₁₋₆ alkyl, C₁₋₆ haloalkyl, C₃₋₆ cycloalkyl, —O(C₁₋₆ alkyl), and —O(C₁₋₆ haloalkyl). In some embodiments, the ring formed by L³ and R², L³ and R⁶, or R² and R⁶ is substituted with two substituents independently selected from halogen, —(C₀₋₆ alkyl)-CN, C₁₋₆ alkyl, C₁₋₆ haloalkyl, C₃₋₆ cycloalkyl, —O(C₁₋₆ alkyl), and —O(C₁₋₆ haloalkyl). In some embodiments, the ring formed by L³ and R², L³ and R⁶, or R² and R⁶ is substituted with three substituents independently selected from halogen, —(C₀₋₆ alkyl)-CN, C₁₋₆ alkyl, C₁₋₆ haloalkyl, C₃₋₆ cycloalkyl, —O(C₁₋₆ alkyl), and —O(C₁₋₆ haloalkyl). In some embodiments, the substituent is C₁₋₆ alkyl, such as —CH₃, —CH₂CH₃, —CH₂CH₂CH₃, —CH(CH₃)₂, —CH₂CH(CH₃)₂, or —C(CH₃)₃.

In some embodiments, the compound of Formula (II) is a compound of Formula (II-a), (II-b), (II-c), or (II-d):

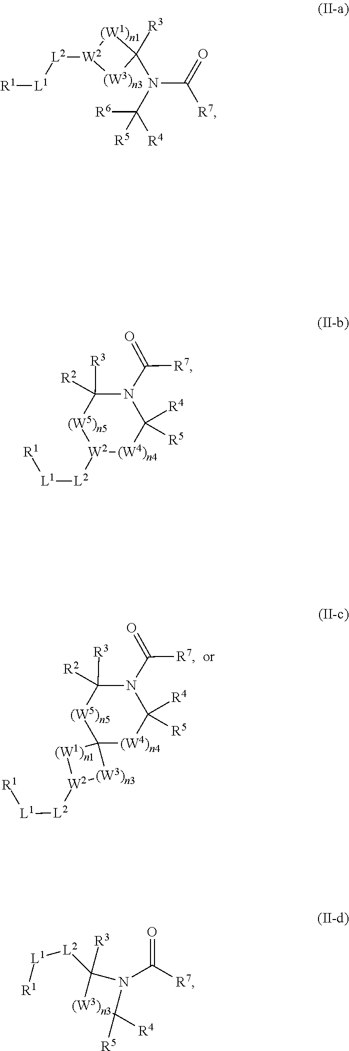

or a pharmaceutically acceptable salt or solvate thereof, wherein:
W¹, w3, W⁴, and W⁵ are each independently selected from N(R¹⁰), C(R¹¹)₂, C(O), O, S(O), and S(O)₂;
W² is selected from N and C(R¹¹);
n1 and n3 are each independently selected from 0, 1, 2, 3, 4, and 5;
n4 and n5 are each independently selected from 0, 1, 2, 3, 4, and 5;
R¹⁰ is independently selected at each occurrence from hydrogen, C₁₋₆ alkyl, and C₃₋₆ cycloalkyl, wherein C₁₋₆ alkyl and C₃₋₆ cycloalkyl are optionally substituted with one, two, or three substituents selected from halogen, —OH, —CN, C₁₋₃ alkyl, C₁₋₃ haloalkyl, C₃₋₆ cycloalkyl, —O(C₁₋₃ alkyl), and —O(C₁₋₃ haloalkyl); and
R¹¹ is independently selected at each occurrence from hydrogen, halogen, —OH, —CN, C₁₋₆ alkyl, C₃₋₆ cycloalkyl, —O(C₁₋₆ alkyl), and —O(C₃₋₆ cycloalkyl), or two R¹¹ attached to the same carbon atom form C₃₋₆ cycloalkyl, wherein C₁₋₆ alkyl, C₃₋₆ cycloalkyl, —O(C₁₋₆ alkyl), and —O(C₃₋₆ cycloalkyl) are optionally substituted with one, two, or three substituents selected from halogen, —OH, —CN, C₁₋₃ alkyl, C₁₋₃ haloalkyl, C₃₋₆ cycloalkyl, —O(C₁₋₃ alkyl), and —O(C₁₋₃ haloalkyl).

In some embodiments, for a compound of Formula (II-a) or a modified protein of Formula (I′-a) or (I-a), W¹ and W³ are each independently selected from N(R¹⁰), C(R¹¹)₂, C(O), O, S(O), and S(O)₂; W² is selected from N and C(R¹¹); n1 and n3 are each independently selected from 0, 1, 2, 3, 4, and 5, wherein the sum of n1 and n3 is at least 1; R¹⁰ is independently selected at each occurrence from hydrogen, —(C₁₋₆ alkyl)-CN, C₁₋₆ alkyl, C₁₋₆ haloalkyl, and C₃₋₆ cycloalkyl; and R¹¹ is independently selected at each occurrence from hydrogen, halogen, —(C₀₋₆ alkyl)-CN, C₁₋₆ alkyl, C₁₋₆ haloalkyl, C₃₋₆ cycloalkyl, —O(C₁₋₆ alkyl), and —O(C₁₋₆ haloalkyl) or two R¹¹ attached to the same carbon atom form C₃₋₆ cycloalkyl. In some embodiments, W¹ and W³ are each C(R¹¹)₂. In some embodiments, W² is N. In some embodiments, W¹ and W³ are each C(R¹¹)₂; and W² is N. In some embodiments, W¹ and W³ are each C(R¹¹)₂; W² is N; n1 is 2 or 3; and n3 is 1 or 2. In some embodiments, W¹ and W³ are each C(R¹¹)₂; W² is N; n1 is 2 or 3; n3 is 1 or 2; and L² is a bond. In some embodiments, W¹ and W³ are each C(R¹¹)₂; W² is N; n1 is 2; and n3 is 1. In some embodiments, W¹ and W³ are each C(R¹¹)₂; W² is N; n1 is 2; and n3 is 2. In some embodiments, W¹ and W³ are each C(R¹¹)₂; W² is N; n1 is 3; and n3 is 2. In some embodiments, L² is a bond. In some embodiments, the sum of n1 and n3 is 2, 3, 4 or 5. In some embodiments, n1 is 2, 3, or 4 and n3 is 1 or 2. In some embodiments,

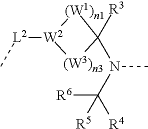

is selected from

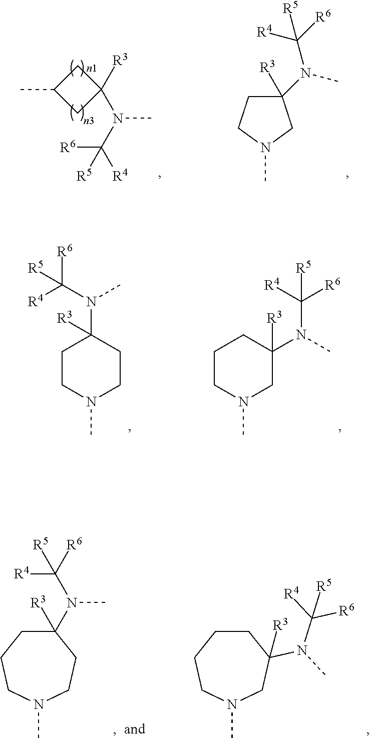

each of which is optionally substituted with one, two, or three R¹¹.

In some embodiments, for a compound of Formula (II-b) or a modified protein of Formula (I′-b) or (I-b), W⁴ and W⁵ are each independently selected from N(R¹⁰), C(R¹¹)₂, C(O), O, S(O), and S(O)₂; W² is selected from N and C(R¹¹); n4 and n5 are each independently selected from 0, 1, 2, 3, 4, and 5; R¹⁰ is independently selected at each occurrence from hydrogen, —(C₁₋₆ alkyl)-CN, C₁₋₆ alkyl, C₁₋₆ haloalkyl, and C₃₋₆ cycloalkyl; and R¹¹ is independently selected at each occurrence from hydrogen, halogen, —(C₀₋₆ alkyl)-CN, C₁₋₆ alkyl, C₁₋₆ haloalkyl, C₃₋₆ cycloalkyl, —O(C₁₋₆ alkyl), and —O(C₁₋₆ haloalkyl) or two R¹¹ attached to the same carbon atom form C₃₋₆ cycloalkyl. In some embodiments, W⁴ and W⁵ are each C(R¹¹)₂. In some embodiments, W² is C(R¹¹). In some embodiments, W⁴ and W⁵ are each C(R¹¹)₂; and W² is C(R¹¹). In some embodiments, W⁴ and WE are each CH₂; and W² is CH. In some embodiments, W⁴ and W⁵ are each C(R¹¹)₂; W² is C(R¹¹); n4 is 0, 1, or 2; and n5 is 0, 1, or 2. In some embodiments, W⁴ and W⁵ are each C(R¹¹)₂; W² is C(R¹¹); and the sum of n4 and n5 is 0, 1 or 2. In some embodiments, W⁴ and W⁵ are each C(R¹¹)₂; W² is C(R¹¹); the sum of n4 and n5 is 0, 1 or 2; and L² is selected from —N(H)C₁₋₃ alkylene-, —N(C₁₋₃ alkyl) C₁₋₃ alkylene-, and —N(R¹⁹)—. In some embodiments, W⁴ and W⁵ are each C(R¹¹)₂; W² is C(R¹¹); n4 is 0; and n5 is 0. In some embodiments, L² is selected from C₁₋₃ alkylene, 2- to 3-membered heteroalkylene, and —N(R¹⁹)—. In some embodiments, L² is selected from 2- to 3-membered heteroalkylene and —N(R¹⁹)—. In some embodiments, L² is selected from —N(H)C₁₋₃ alkylene-, —N(C₁₋₃ alkyl)C₁₋₃ alkylene-, and —N(R¹⁹)—. In some embodiments, L² is selected from —N(R¹⁹)—, —N(CH₃)CH₂—, and —N(CH₃)CH(CH₃)—. In some embodiments, the sum of n4 and n5 is 0 or 1. In some embodiments, n4 and n5 are each 0. In some embodiments,

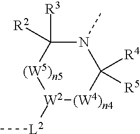

is selected from

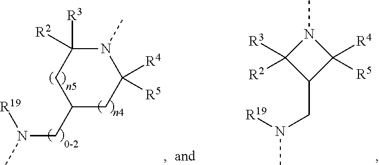

each of which is optionally substituted with one, two, or three R¹¹.

In some embodiments, for a compound of Formula (II-c) or a modified protein of Formula (I′-c) or (I-c), W¹, W³, W⁴, and W⁵ are each independently selected from N(R¹⁰), C(R¹¹)₂, C(O), O, S(O), and S(O)₂; W² is selected from N and C(R¹¹); n1 and n3 are each independently selected from 0, 1, 2, 3, 4, and 5, wherein the sum of n1 and n3 is at least 2; n4 and n5 are each independently selected from 0, 1, 2, 3, 4, and 5; R¹⁰ is independently selected at each occurrence from hydrogen, —(C₁₋₆ alkyl)-CN, C₁₋₆ alkyl, C₁₋₆ haloalkyl, and C₃₋₆ cycloalkyl; and R¹¹ is independently selected at each occurrence from hydrogen, halogen, —(C₀₋₆ alkyl)-CN, C₁₋₆ alkyl, C₁₋₆ haloalkyl, C₃₋₆ cycloalkyl, —O(C₁₋₆ alkyl), and —O(C₁₋₆ haloalkyl) or two R¹¹ attached to the same carbon atom form C₃₋₆ cycloalkyl. In some embodiments, each W¹ is independently selected from C(R¹¹)₂ and O. In some embodiments, n1 is 2, 3, or 4, one W¹ is O, and the remaining W¹ are each C(R¹¹)₂. In some embodiments, W³ is C(R¹¹)₂. In some embodiments, W² is N. In some embodiments, W⁴ and W⁵ are each C(R¹¹)₂. In some embodiments, n1 is 2, 3, or 4; one W¹ is selected from C(R¹¹)₂ and O and the remaining W¹ are each C(R¹¹)₂; W² is N; n3 is 1 or 2; W³ is C(R¹¹)₂; n4 is 0 or 1; W⁴ is C(R¹¹)₂; n5 is 0 or 1; and W⁵ is C(R¹¹)₂. In some embodiments, n1 is 2, 3, or 4; one W¹ is selected from C(R¹¹)₂ and O and the remaining W¹ are each C(R¹¹)₂; W² is N; n3 is 1 or 2; W³ is C(R¹¹)₂; n4 is 0 or 1; W⁴ is C(R¹¹)₂; n5 is 0 or 1; W⁵ is C(R¹¹)₂; and L² is a bond. In some embodiments, n1 is 2, 3, or 4; one W¹ is selected from CH₂ and O and the remaining W¹ are each CH₂; W² is N; n3 is 1 or 2; W³ is CH₂; n4 is 0 or 1; W⁴ is CH₂; n5 is 0 or 1; and W⁵ is CH₂. In some embodiments, the sum of n1 and n3 is 3, 4, or 5; one W¹ is selected from C(R¹¹)₂ and O and the remaining W¹ are each C(R¹¹)₂; W² is N; W³ is C(R¹¹)₂; n4 is 0; and n5 is 0. In some embodiments, n1 is 2, 3, or 4; W¹ is C(R¹¹)₂; W² is N; n3 is 1 or 2; W³ is C(R¹¹)₂; n4 is 0 or 1; W⁴ is C(R¹¹)₂; n5 is 0 or 1; and W⁵ is C(R¹¹)₂. In some embodiments, n1 is 4; W¹ is C(R¹¹)₂; W² is N; n3 is 1; W³ is C(R¹¹)₂; n4 is 0; and n5 is 0. In some embodiments, n1 is 3; W¹ is C(R¹¹)₂; W² is N; n3 is 1; W³ is C(R¹¹)₂; n4 is 0; and n5 is 0. In some embodiments, n1 is 3; one W¹ is selected from C(R¹¹)₂ and O and the remaining W¹ are each C(R¹¹)₂; W² is N; n3 is 1; W³ is C(R¹¹)₂; n4 is 0; and n5 is 0. In some embodiments, n1 is 2; W¹ is C(R¹¹)₂; W² is N; n3 is 1; W³ is C(R¹¹)₂; n4 is 0; and n5 is 0. In some embodiments, L² is a bond. In some embodiments, the sum of n1 and n3 is 2, 3, 4, or 5. In some embodiments, n1 is 2, 3, or 4 and n3 is 1. In some embodiments, the sum of n4 and n5 is 0 or 1. In some embodiments, n4 and n5 are each 0. In some embodiments,

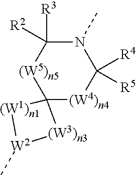

is selected from

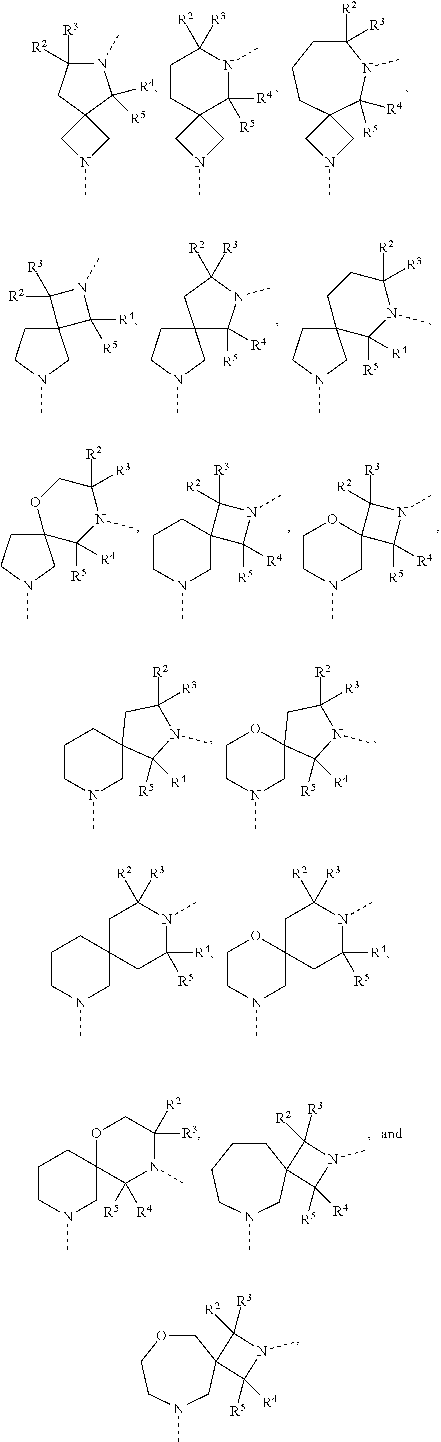

each of which is optionally substituted with one, two, or three R¹¹.

In some embodiments, for a compound of Formula (II-d) or a modified protein of Formula (I′-d) or (I-d), W³ is selected from N(R¹⁰), C(R¹¹)₂, C(O), O, S(O), and S(O)₂; n3 is selected from 0, 1, 2, 3, 4, and 5; R¹⁰ is independently selected at each occurrence from hydrogen, —(C₁₋₆ alkyl)-CN, C₁₋₆ alkyl, C₁₋₆ haloalkyl, and C₃₋₆ cycloalkyl; and R¹¹ is independently selected at each occurrence from hydrogen, halogen, (C₀₋₆ alkyl)-CN, C₁₋₆ alkyl, C₁₋₆ haloalkyl, C₃₋₆ cycloalkyl, —O(C₁₋₆ alkyl), and —O(C₁₋₆ haloalkyl). In some embodiments, each W³ is C(R¹¹)₂. In some embodiments, W³ is C(R¹¹)₂; and n3 is 1, 2, or 3. In some embodiments, W³ is C(R¹¹)₂; n3 is 1, 2, or 3; and L² is selected from —N(H)C₁₋₃ alkylene-, —N(C₁₋₃ alkyl)C₁₋₃ alkylene-, and —N(R¹⁹)—. In some embodiments, W³ is CH₂; and n3 is 1, 2, or 3. In some embodiments, W³ is C(R¹¹)₂; and n3 is 1. In some embodiments, W³ is C(R¹¹)₂; and n3 is 2. In some embodiments, W³ is C(R¹¹)₂; and n3 is 3. In some embodiments, L² is selected from C₁₋₃ alkylene, 2- to 3-membered heteroalkylene, and —N(R¹⁹)—. In some embodiments, L² is selected from 2- to 3-membered heteroalkylene and —N(R¹⁹)—. In some embodiments, L² is selected from —N(H)C₁₋₃ alkylene-, —N(C₁₋₃ alkyl) C₁₋₃ alkylene-, and —N(R¹⁹)—. In some embodiments, L² is selected from —N(R¹⁹)—, —N(CH₃)CH₂—, and —N(CH₃)CH(CH₃)—. In some embodiments, n3 is 1, 2, or 3. In some embodiments,

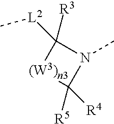

is selected from

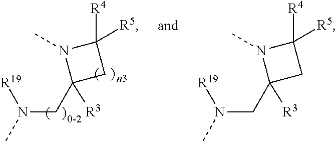

each of which is optionally substituted with one, two, or three R¹¹.

In some embodiments, for a compound of Formula (II-a), (II-b), (II-c), or (II-d) or a modified protein of Formula (I′-a), (I′-b), (I′-c), (I′-d), (I-a), (I-b), (I-c), or (I-d), R¹⁰ and R¹¹ are independently selected at each occurrence from hydrogen and C₁₋₃ alkyl. In some embodiments, one R¹⁰ or R¹¹ is C₁₋₃ alkyl, such as —CH₃, —CH₂CH₃, or —CH(CH₃)₂, and any remaining R¹⁰ and R¹¹ are each hydrogen. In some embodiments, two R¹⁰ and/or R¹¹ are independently C₁₋₃ alkyl, such as —CH₃, —CH₂CH₃, or —CH(CH₃)₂, and any remaining R¹⁰ and R¹¹ are each hydrogen.

In some embodiments, for a compound of Formula (II), (II-a), (II-b), (II-c), or (II-d) or a modified protein of Formula (I′), (I′-a), (I′-b), (I′-c), (I′-d), (I), (I-a), (I-b), (I-c), or (I-d), R¹ is selected from C₆₋₁₀ aryl and 5- to 10-membered heteroaryl, each of which is optionally substituted with one, two, three, four, or five R²⁰. In some embodiments, R¹ is selected from naphthyl, isoquinolinyl, indazolyl, benzothiazolyl, benzothiophenyl, phenyl, and pyridinyl, each of which is optionally substituted with one or more R²⁰. In some embodiments, R¹ is substituted with one, two, three, or four substituents independently selected from halogen, —CN, C₁₋₃ alkyl, C₁₋₃ haloalkyl, C₂₋₃ alkenyl, C₂₋₃ alkynyl, —OR²², —N(R²²)(R²³), and C₃₋₆ cycloalkyl. In some embodiments, R¹ is substituted with one, two, three, or four substituents independently selected from halogen, —CN, —CH₃, —CH₂CH₃, —CH—CH₂, —CF₃, —C═C, —OH, —NH₂, and -cyclopropyl.

In some embodiments, for a compound of Formula (II), (II-a), (II-b), (II-c), or (II-d) or a modified protein of Formula (I′), (I′-a), (I′-b), (I′-c), (I′-d), (I), (I-a), (I-b), (I-c), or (I-d), R¹ is selected from fused bicyclic C₄₋₁₂ cycloalkyl, fused bicyclic C₃₋₁₁ heterocycloalkyl, fused bicyclic C₇₋₁₂ aryl, and fused bicyclic C₃₋₁₁ heteroaryl, wherein the fused bicyclic C₄₋₁₂ cycloalkyl, fused bicyclic C₃₋₁₁ heterocycloalkyl, fused bicyclic C₇₋₁₂ aryl, and fused bicyclic C₃₋₁₁ heteroaryl are optionally substituted with one, two, three, four, five, six, or seven R²⁰. In some embodiments, R¹ is selected from spirocyclic bicyclic C₄₋₁₂ cycloalkyl and spirocyclic bicyclic C₃₋₁₁ heterocycloalkyl wherein the spirocyclic bicyclic C₄₋₁₂ cycloalkyl and spirocyclic bicyclic C₃₋₁₁ heterocycloalkyl are optionally substituted with one, two, three, four, five, six, or seven R²⁰. In some embodiments, R¹ is a polycyclic ring system.

In some embodiments, for a compound of Formula (II), (II-a), (II-b), (II-c), or (II-d) or a modified protein of Formula (I′), (I′-a), (I′-b), (I′-c), (I′-d), (I), (I-a), (I-b), (I-c), or (I-d), R¹ is substituted with one, two, three, or four substituents independently selected from halogen, —CN, C₁₋₆ alkyl, C₂₋₆ alkenyl, C₂₋₆ alkynyl, C₃₋₆ cycloalkyl, —OR²², —SR²², and —N(R²²)(R²³), wherein C₁₋₆ alkyl, C₂₋₆ alkenyl, C₂₋₆ alkynyl, and C₃₋₆ cycloalkyl are optionally substituted with one, two, or three substituents independently selected from halogen, C₁₋₆ alkyl, C₁₋₆ haloalkyl, and —OR²². In some embodiments, R¹ is substituted with one, two, three, or four substituents independently selected from halogen, —CN, C₁₋₃ alkyl, C₂₋₃ alkenyl, C₂₋₃ alkynyl, —OR²², and —N(R²²)(R²³). In some embodiments, R¹ is substituted with one, two, three, or four substituents independently selected from halogen, —CN, —CH₃, —C═CH, —OH, and —NH₂. In some embodiments, R¹ is substituted with —F, —CN, and —NH₂. In some embodiments, R¹ is substituted with —F, —C═CH, and —OH. In some embodiments, R¹ is substituted with —CF₃, —CH₃, and —NH₂. In some embodiments, R¹ is substituted with —CF₃ and —NH₂. In some embodiments, R¹ is substituted with —CF₃, —CH₃, —F, and —NH₂. In some embodiments, R¹ is substituted with —CF₃, —F, and —NH₂. In some embodiments, R¹ is substituted with one, two, three, or four substituents independently selected from halogen, —CN, —CH₃, —CH₂CH₃, —CH—CH₂, —CF₃, —C═C, —OH, —NH₂, and -cyclopropyl.

In some embodiments, for a compound of Formula (II), (II-a), (II-b), (II-c), or (II-d) or a modified protein of Formula (I′), (I′-a), (I′-b), (I′-c), (I′-d), (I), (I-a), (I-b), (I-c), or (I-d), R¹ is selected from:

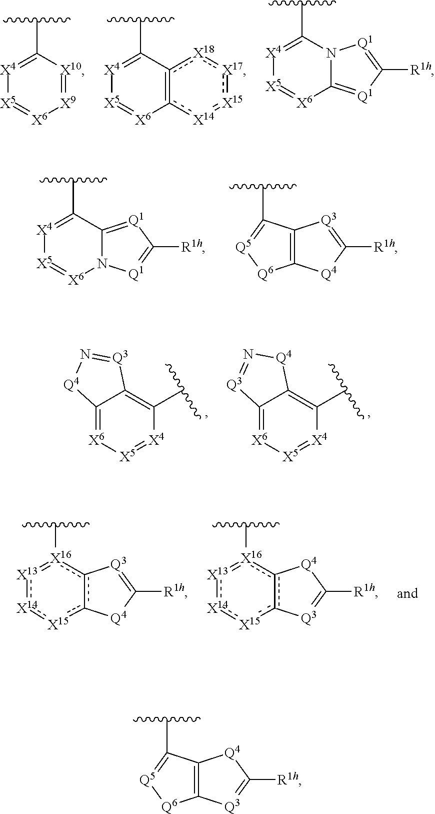

wherein:
Q¹, Q³, and Q⁵ are independently selected from N and C(R^{1d});
Q⁴ and Q⁶ are independently selected from O, S, C(R^{1a})(R^{1d}), and N(R^{1c});
X⁴, X⁵, X⁶, X⁹, and X¹⁰ are independently selected from C(R^{1a}) and N;
X¹³ is selected from a bond, C(R^{1a}), N, C(O), C(R^{1a})(R^{1b}), C(O)C(R^{1a})(R^{1b}), C(R^{1a})(R^{1b})C(R^{1a})(R^{1b}), C(R^{1a})(R^{1b}) N(R^{1c}), and N(R^{1c});
X¹⁴, X¹⁵, X¹⁷, and X¹⁸ are independently selected from C(O), C(R^{1a}), N, C(R^{1a})(R^{1b}), and N(R^{1c});
X¹⁶ is selected from C, N, and C(R^{1a});
each R^{1a}, R^{1b}, R^{1d}, and R^{1h} is independently selected from hydrogen, halogen, —CN, C₁₋₆ alkyl, C₁₋₆ haloalkyl, C₂₋₆ alkenyl, C₂₋₆ alkynyl, C₃₋₁₀ carbocycle, 3- to 10-membered heterocycle, —OR¹², —SR¹², —N(R¹²)(R¹³), —C(O)OR¹², —OC(O)N(R¹²)(R¹³), —N(R¹²)C(O)N(R¹²)(R¹³), —N(R¹²)C(O)OR¹², —N(R¹²)S(O)₂R¹², —C(O)R¹², —S(O)R¹², —OC(O)R¹², —C(O)N(R¹²)(R¹³), —C(O)C(O)N(R¹²)(R¹³), —N(R¹²)C(O)R¹², —S(O)₂R¹², —S(O)₂N(R¹²)(R¹³), —S(═O)(═NH) N(R¹²)(R¹³), —CH₂C(O)N(R¹²)(R¹³), —CH₂N(R¹²)C(O)R¹², —CH₂S(O)₂R¹², and —CH₂S(O)₂N(R¹²)(R¹³), wherein C₁₋₆ alkyl, C₂₋₆ alkenyl, C₂₋₆ alkynyl, C₃₋₁₀ carbocycle, and 3- to 10-membered heterocycle are optionally substituted with one, two, or three R²⁰; or R¹ª and R¹b bonded to the same carbon are joined to form 3- to 10-membered heterocycle or C₃₋₁₀ carbocycle, wherein 3- to 10-membered heterocycle and C₃₋₁₀ carbocycle are optionally substituted with one, two, or three R²⁰; or two R¹ª bonded to adjacent atoms are joined to form 3- to 10-membered heterocycle or C₃₋₁₀ carbocycle, wherein 3- to 10-membered heterocycle and C₃₋₁₀ carbocycle are optionally substituted with one, two, or three R²⁰; or R^{1h} and one of R^{1a}, R^{1b}, R^{1c}, and R^{1d} bonded to adjacent atoms are joined to form 3- to 10-membered heterocycle or C₃₋₁₀ carbocycle, wherein 3- to 10-membered heterocycle and C₃₋₁₀ carbocycle are optionally substituted with one, two, or three R²⁰; and each R^{1c} is independently selected from hydrogen, C₁₋₆ alkyl, C₂₋₆ alkenyl, C₂₋₆ alkynyl, 3- to 10-membered heterocycle, and C₃₋₁₀ carbocycle, wherein C₁₋₆ alkyl, C₂₋₆ alkenyl, C₂₋₆ alkynyl, 3- to 10-membered heterocycle, and C₃₋₁₀ carbocycle are optionally substituted with one, two, or three R²⁰. In some embodiments, R¹ is selected from

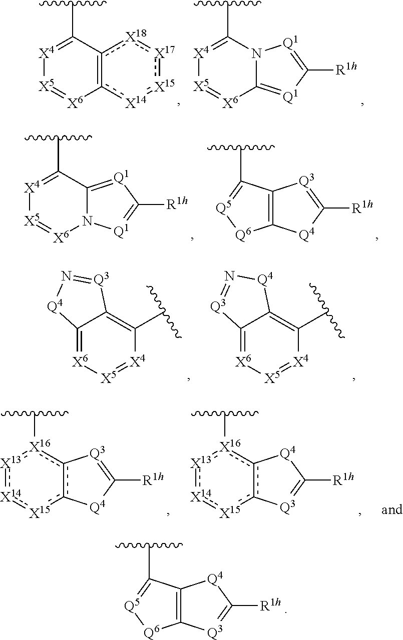

In some embodiments, R¹ is selected from

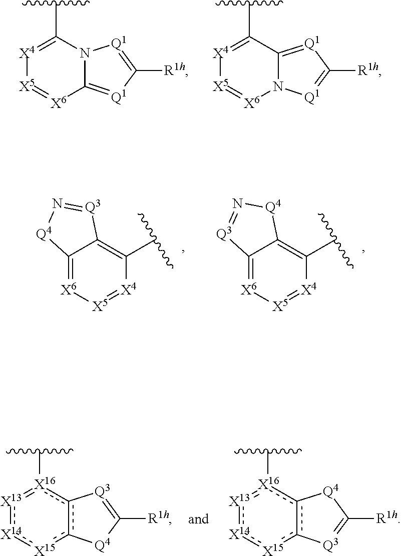

In some embodiments, for a compound of Formula (II), (II-a), (II-b), (II-c), or (II-d) or a modified protein of Formula (I′), (I′-a), (I′-b), (I′-c), (I′-d), (I), (I-a), (I-b), (I-c), or (I-d), R¹ is selected from

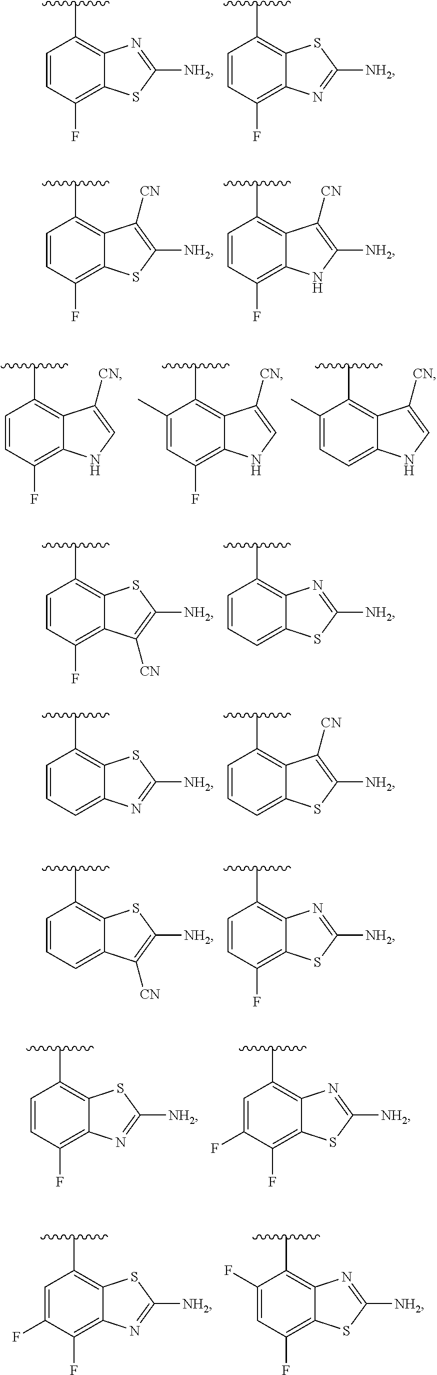

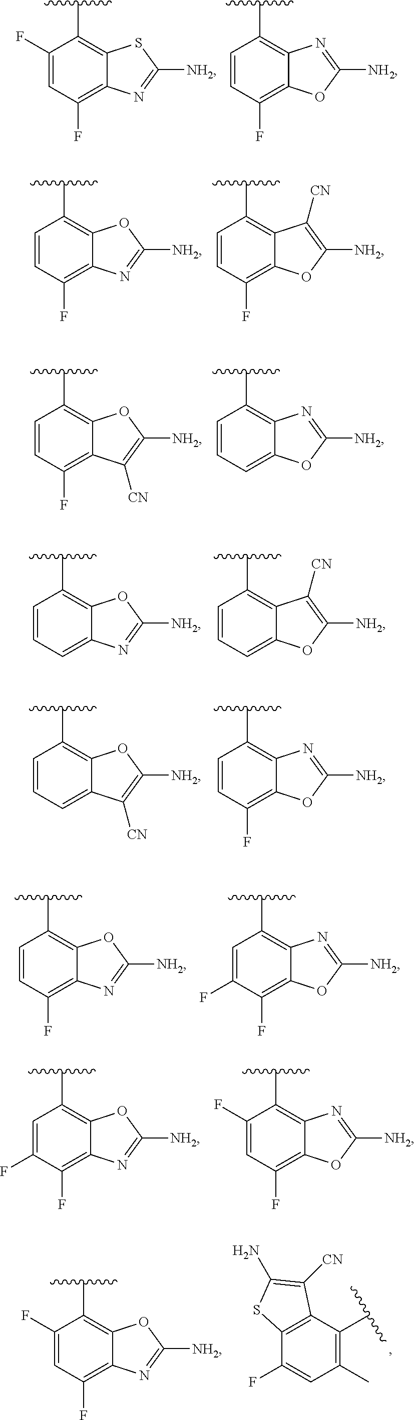

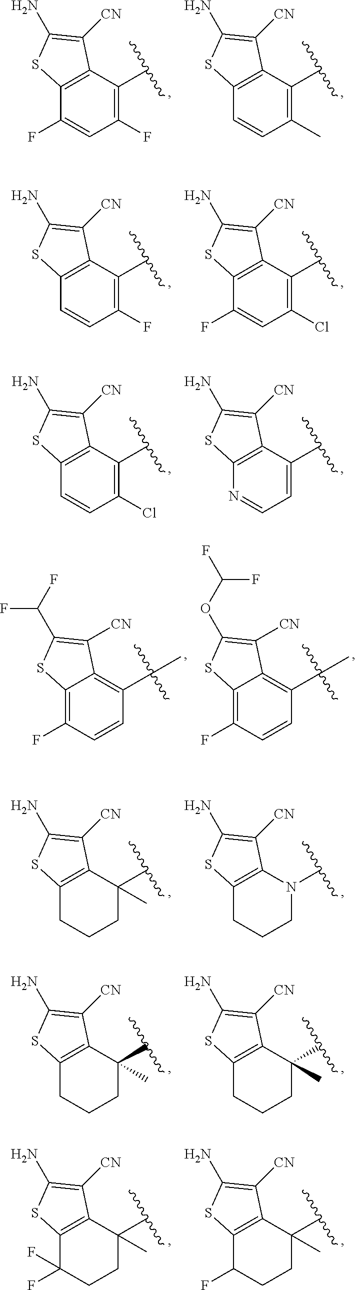

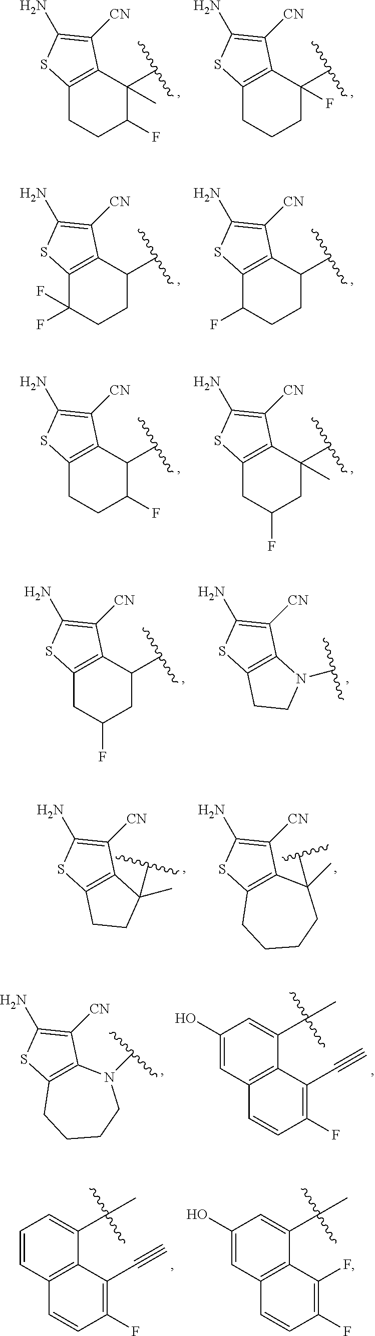

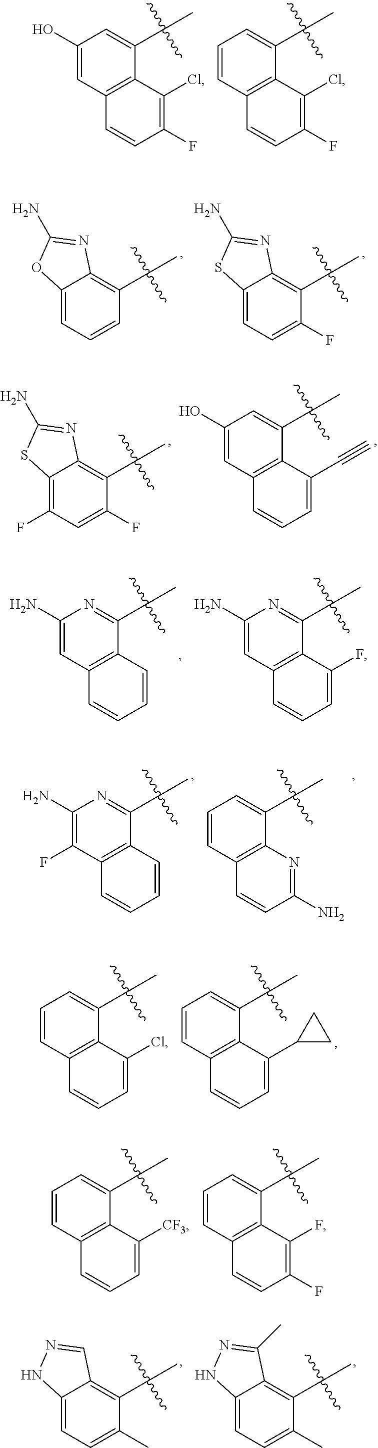

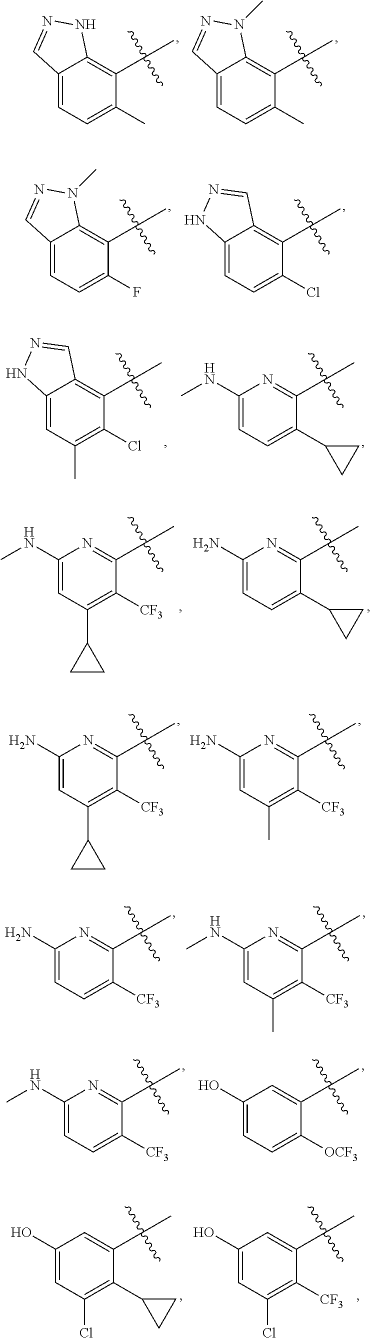

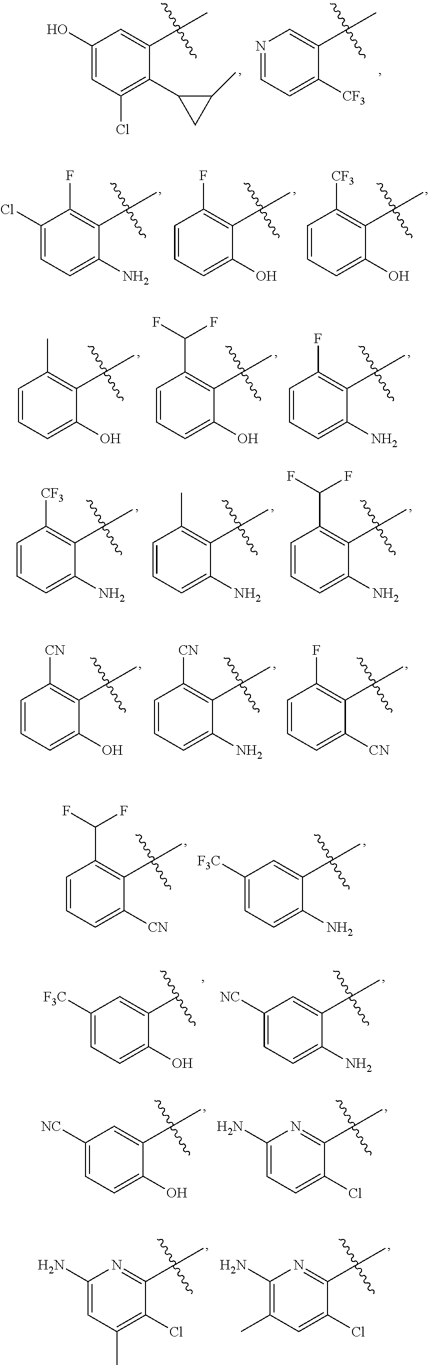

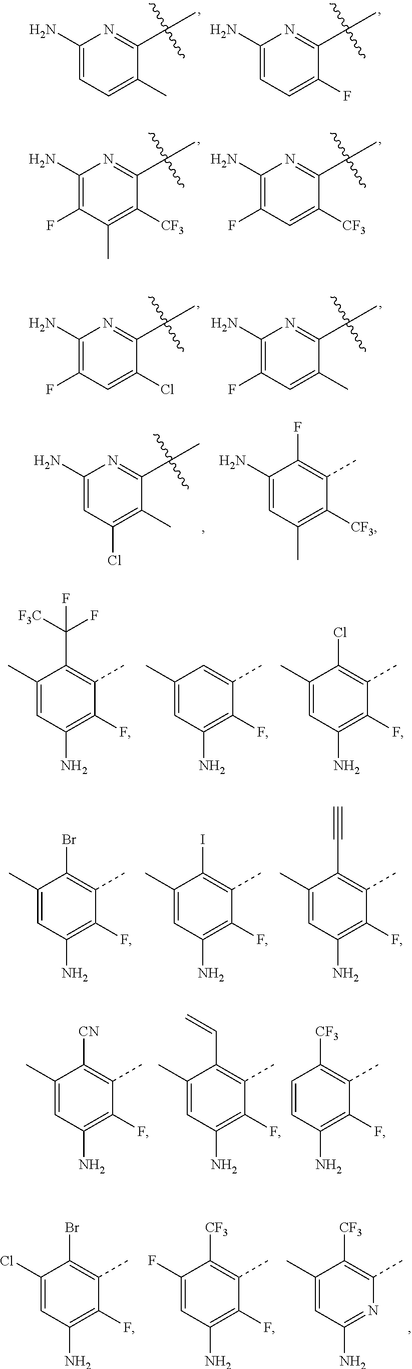

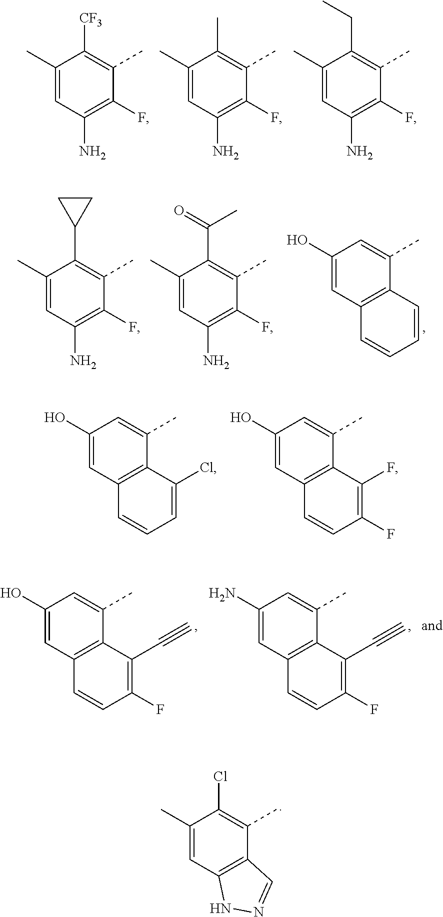

In some embodiments, R¹ is

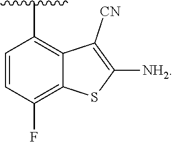 [SMILES: *c1ccc(F)c2sc(N)c(C#N)c12]

In some embodiments, for a compound of Formula (II), (II-a), (II-b), (II-c), or (II-d) or a modified protein of Formula (I′), (I′-a), (I′-b), (I′-c), (I′-d), (I), (I-a), (I-b), (I-c), or (I-d), R¹ is selected from

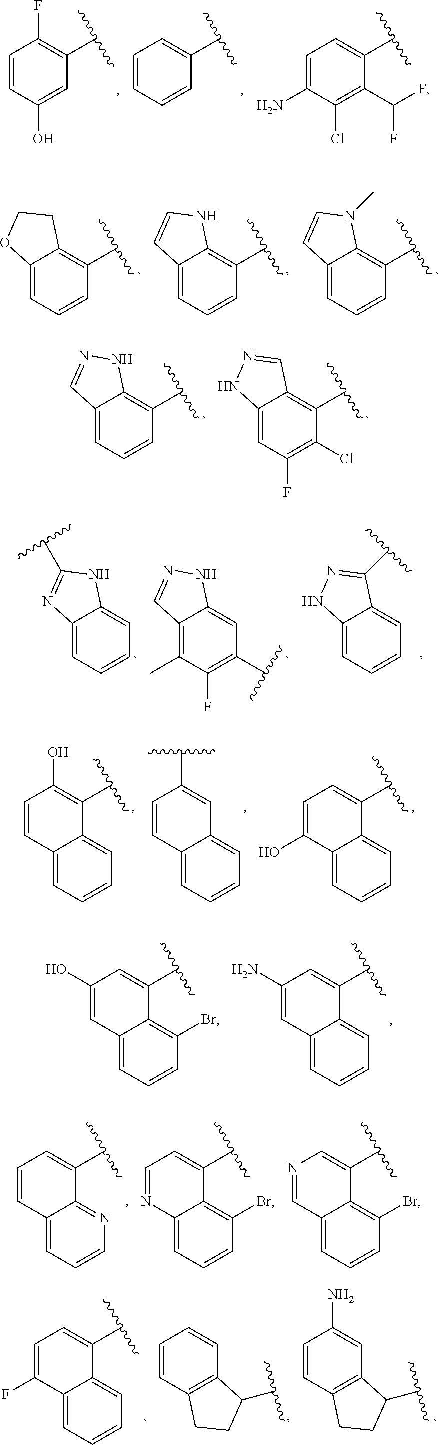

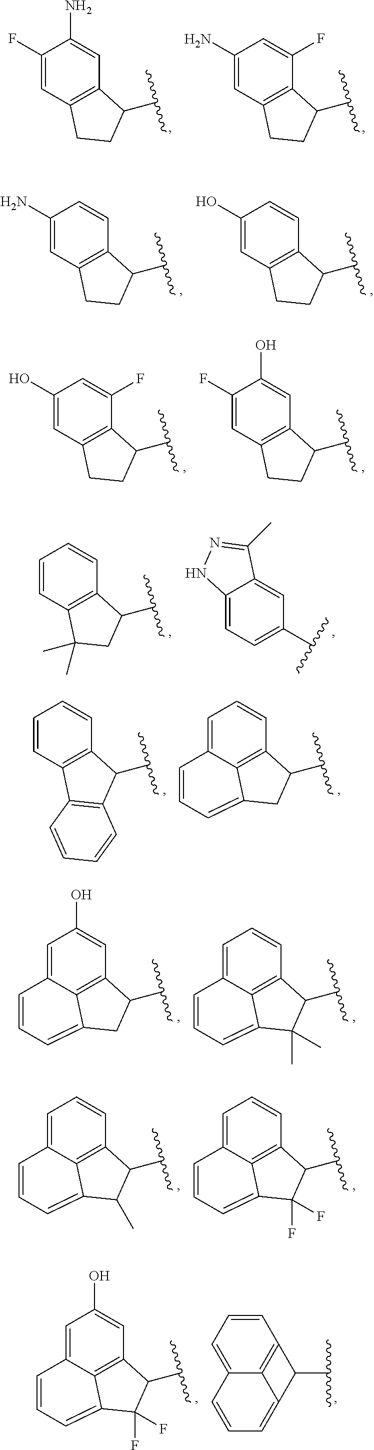

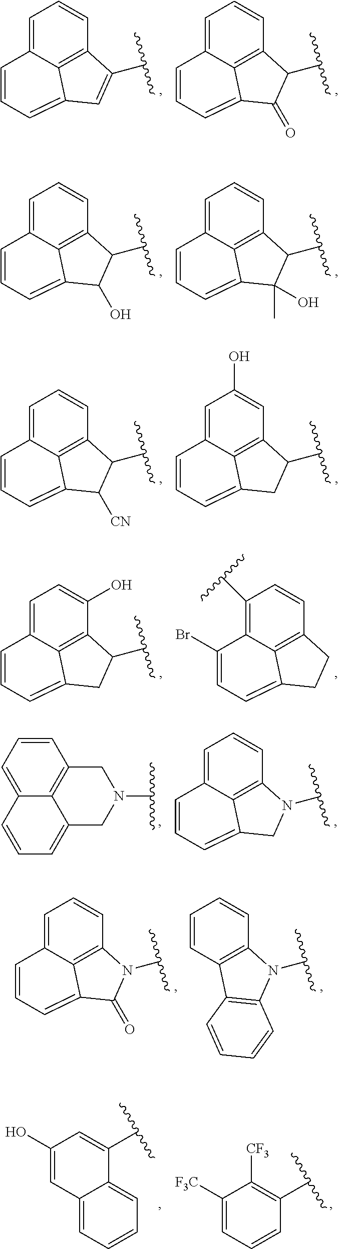

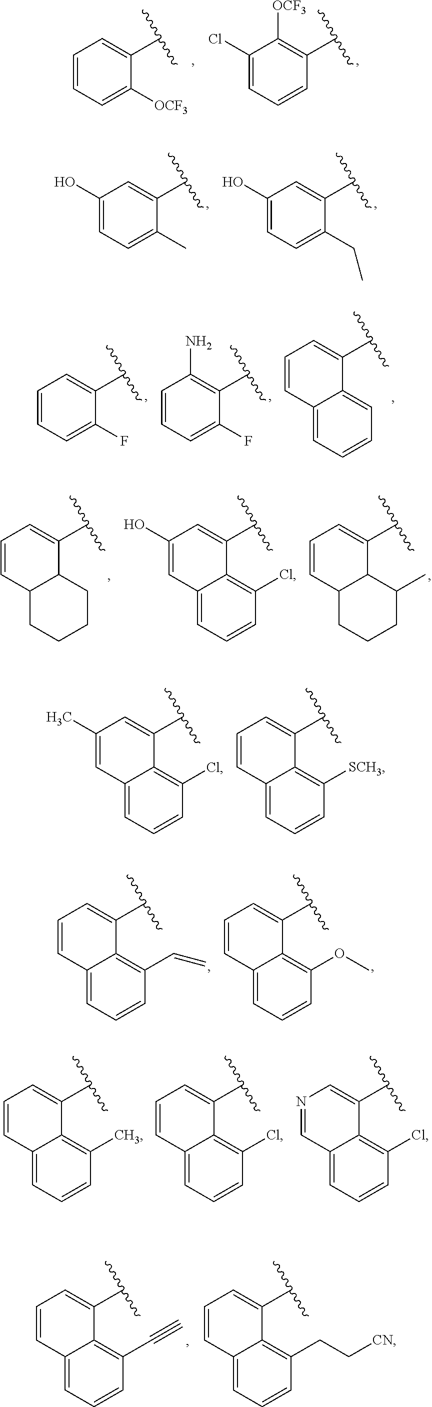

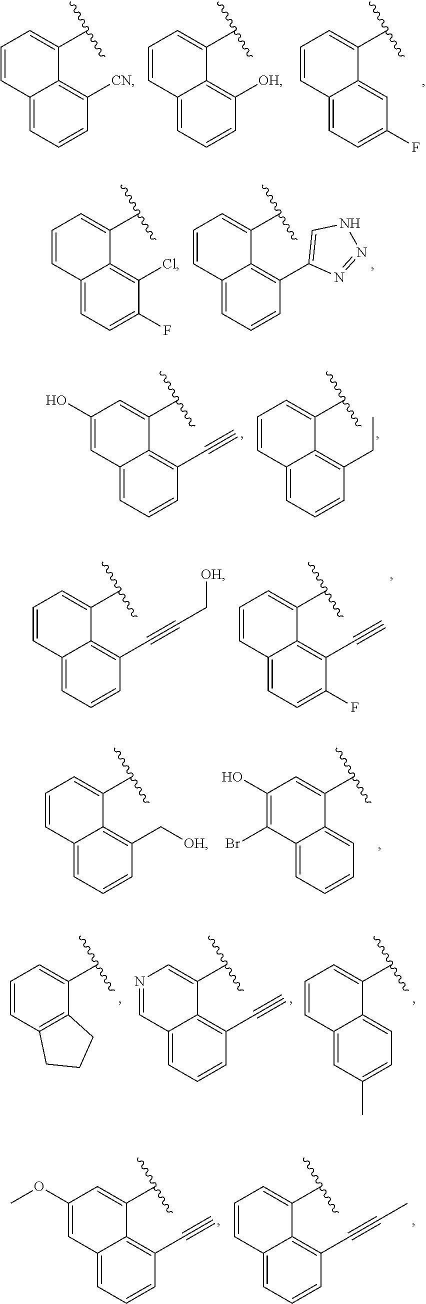

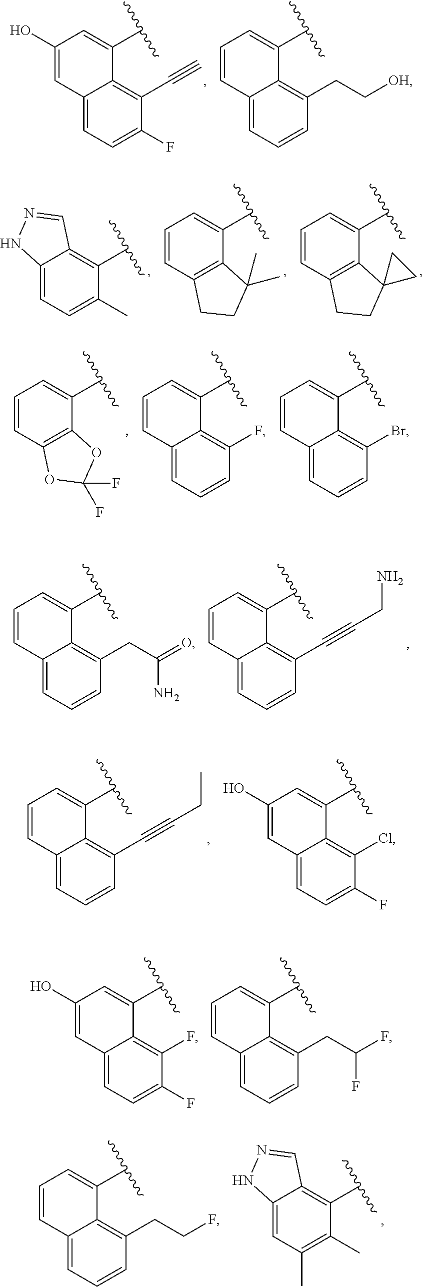

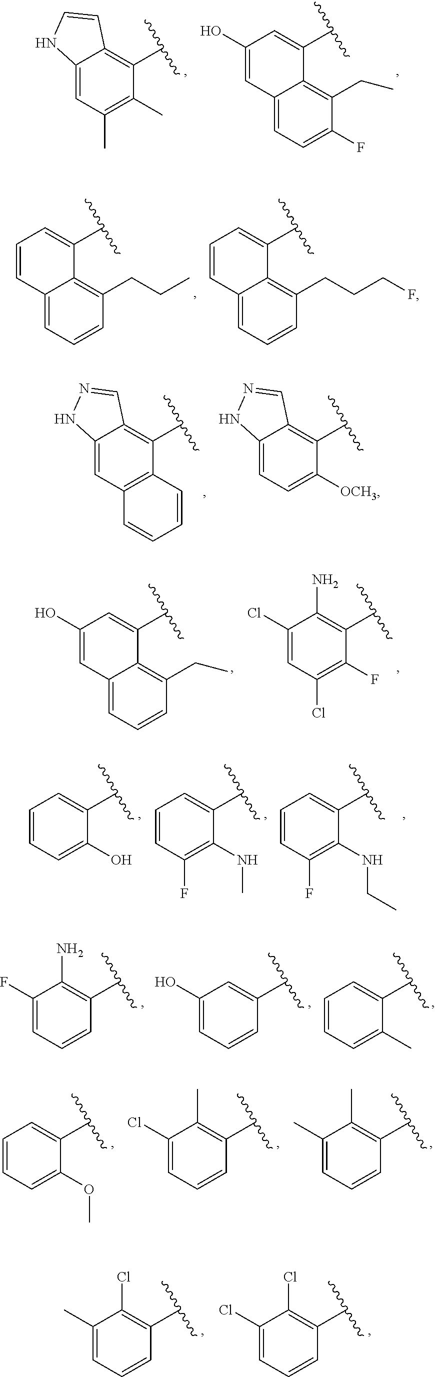

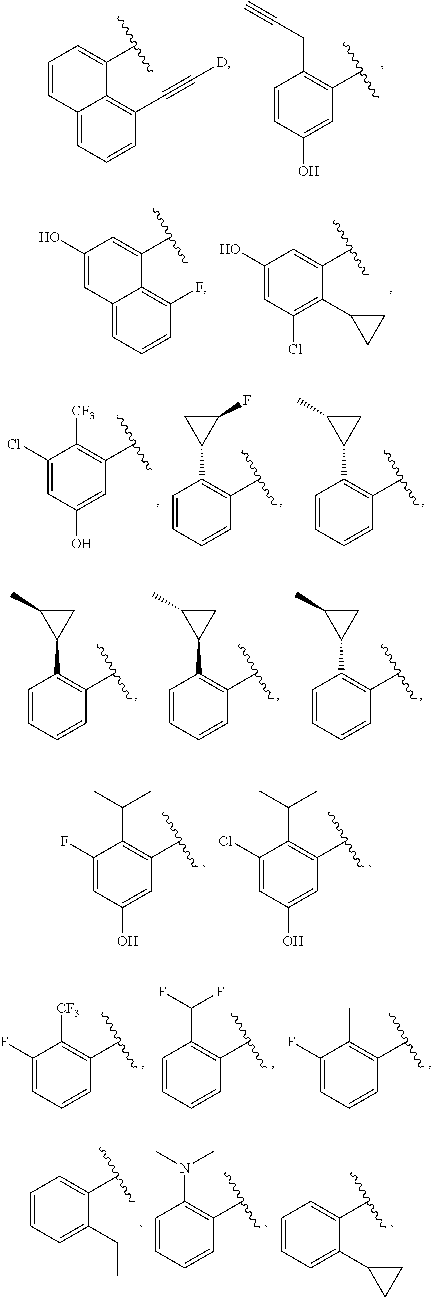

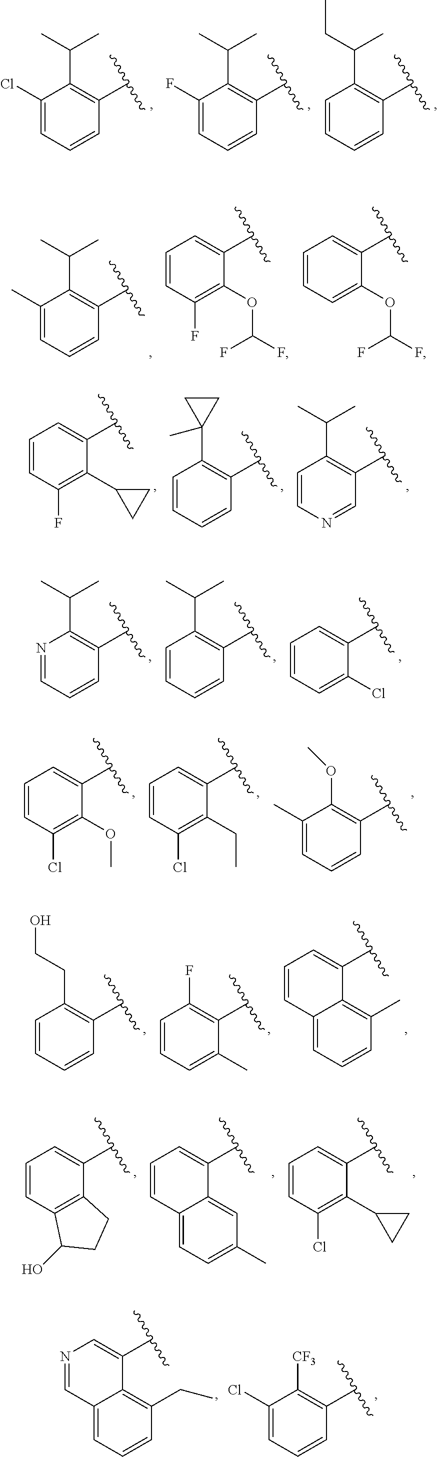

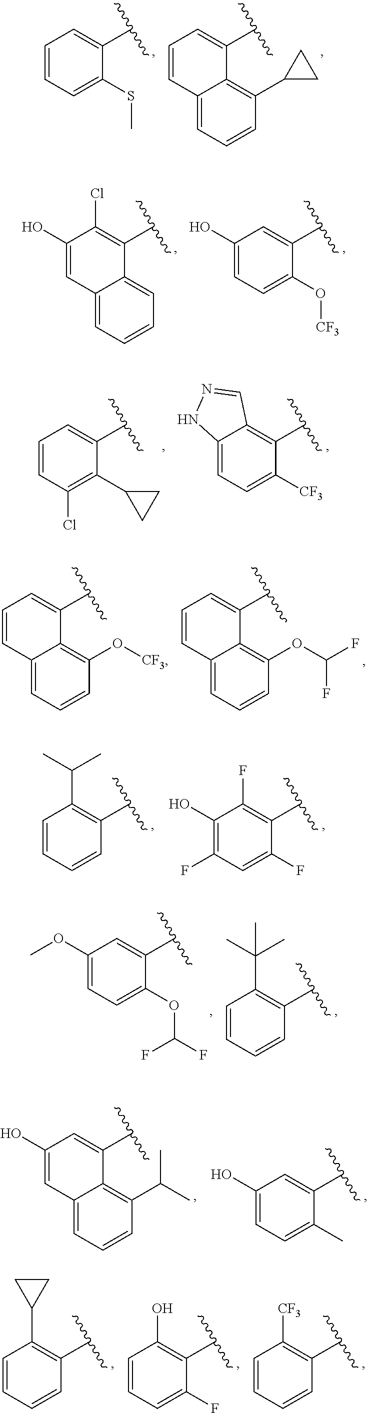

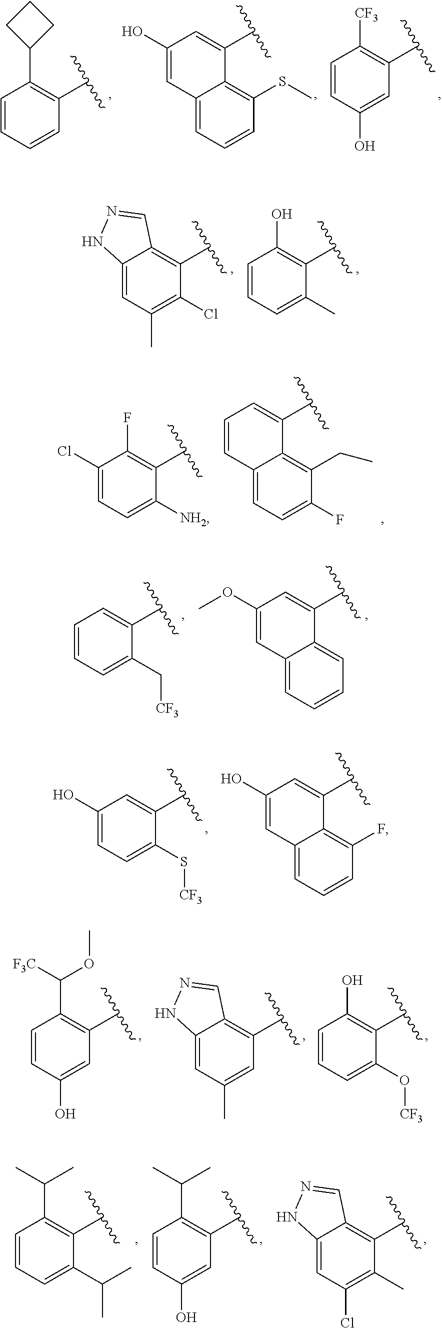

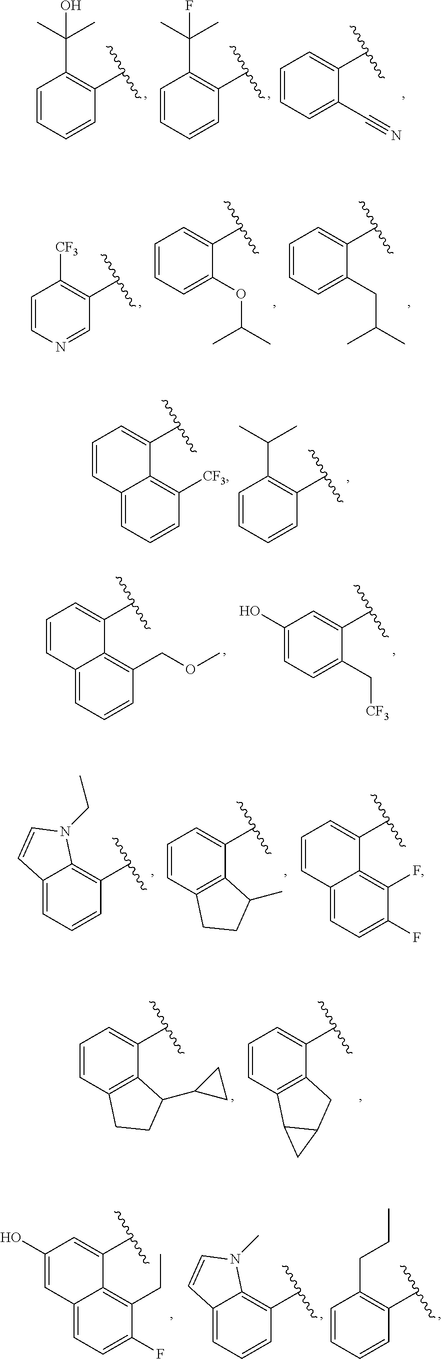

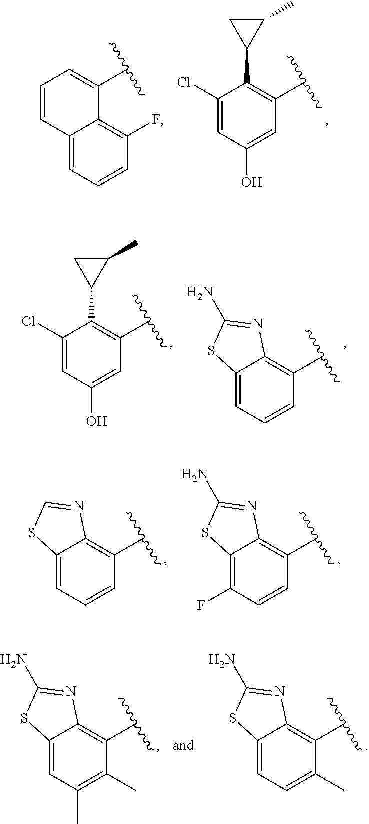

In some embodiments, R¹ is selected from

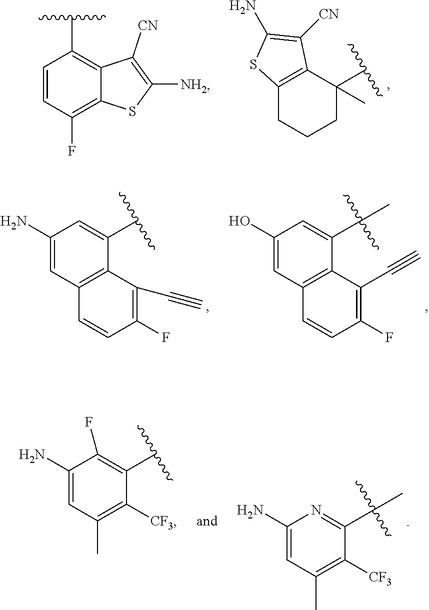

In some embodiments, R¹ is

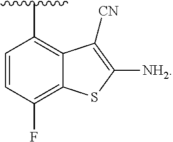 [SMILES: *c1ccc(F)c2sc(N)c(C#N)c12]

In some embodiments, R¹ is

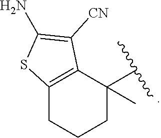 [SMILES: *C1(C)CCCc2sc(N)c(C#N)c21]

In some embodiments, R¹ is

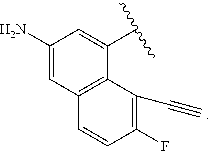 [SMILES: *c1cc(N)cc2ccc(F)c(C#C)c12]

In some embodiments, R¹ is

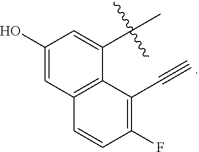 [SMILES: *c1cc(O)cc2ccc(F)c(C#C)c12]

In some embodiments, R¹ is

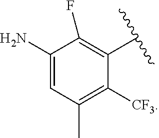 [SMILES: *c1c(F)c(N)cc(C)c1C(F)(F)F]

In some embodiments, R¹ is

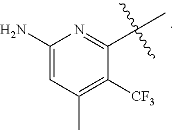 [SMILES: *c1nc(N)cc(C)c1C(F)(F)F]

In some embodiments, for a compound of Formula (II), (II-a), (II-b), (II-c), or (II-d) or a modified protein of Formula (I′), (I′-a), (I′-b), (I′-c), (I′-d), (I), (I-a), (I-b), (I-c), or (I-d), L¹ is 6- to 12-membered heterocycle optionally substituted with one or more R²⁰. In some embodiments, L¹ is 10-membered bicyclic heterocycle substituted with one, two, three, or four R²⁰. In some embodiments, L¹ comprises 1 to 5 nitrogen atoms. In some embodiments, L¹ is 6-membered monocyclic heterocycle optionally substituted with one or more R²⁰. In some embodiments, L¹ is 12- to 20-membered polycyclic heterocycle optionally substituted with one or more R²⁰, such as 12- to 18-membered tricyclic heterocycle optionally substituted with one or more R²⁰. In some embodiments, L¹ is selected from tetrahydropyridopyrimidine (e.g., 5,6,7,8-tetrahydropyrido[3,4-d]pyrimidine), pyridopyrimidine (e.g., pyrido[4,3-d]pyrimidine), pyridopyrimidinone (e.g., pyrido[4,3-d]pyrimidin-5 (6H)-one or pyrido[2,3-d]pyrimidin-2 (1H)-one), pyrimidine, quinazoline, and 1,2,4-oxadiazole.

In some embodiments, for a compound of Formula (II), (II-a), (II-b), (II-c), or (II-d) or a modified protein of Formula (I′), (I′-a), (I′-b), (I′-c), (I′-d), (I), (I-a), (I-b), (I-c), or (I-d), L¹ is:

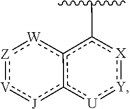

wherein:
W is N, C(R¹⁷), N(R^{17b}), C(R¹⁷)₂, C(O), S(O), or S(O)₂;
Z is N, C(R¹⁷), N(R^{17b}), C(R¹⁷)₂, C(O), S(O), or S(O)₂; wherein W and Z are not both selected from C(O), S(O), and S(O)₂;
V and J are each independently selected from N, C(R¹), C(R¹⁷), N(R¹), N(R^{17b}), C(R¹)(R¹⁷), and C(R¹⁷)₂; wherein exactly one of V and J is C(R¹), N(R¹), or C(R¹)(R¹⁷);
U is N, C(R¹⁷), N(R¹⁷%), C(R¹⁷)₂, S(O), S(O)₂, or C(O);
Y is N, C(R¹⁸), N(R^{17b}), C(R¹⁸)(R¹⁷), S(O), S(O)₂, or C(O);
X is N, C(R¹⁷), N(R^{17b}), or C(R¹⁷)₂;
R¹⁷ is independently selected at each occurrence from hydrogen, halogen, —CN, C₁₋₆ alkyl, C₂₋₆ alkenyl, C₂₋₆ alkynyl, 2- to 6-membered heteroalkyl, 2- to 6-membered heteroalkenyl, 2- to 6-membered heteroalkynyl, —C₀₋₆ alkyl-(C₃₋₁₂ carbocycle), -(2- to 6-membered heteroalkyl)-(C₃₋₁₂ carbocycle), —C₀₋₆ alkyl-(3- to 12-membered heterocycle), -(2- to 6-membered heteroalkyl)-(3- to 12-membered heterocycle), —OR¹², —SR¹², —N(R¹²)(R¹³), —NR¹², ═C(R¹⁴)₂, —C(O)OR¹², —OC(O)N(R¹²)(R¹³), —N(R¹²)C(O)N(R¹²)(R¹³), —N(R¹²)C(O)OR¹², —N(R¹²)S(O)₂R¹², —C(O)R¹², —S(O)R¹², —OC(O)R¹², —C(O)N(R¹²)(R¹³), —C(O)C(O)N(R¹²)(R¹³), —N(R¹²)C(O)R¹², —S(O)₂R¹², —S(O)(NR¹²)R¹², —S(O)₂N(R¹²)(R¹³), —S(═O)(═NR¹²)N(R¹²)(R¹³), and —OCH₂C(O)OR¹², wherein C₁₋₆ alkyl, C₂₋₆ alkenyl, C₂₋₆ alkynyl, 2- to 6-membered heteroalkyl, 2- to 6-membered heteroalkenyl, 2- to 6-membered heteroalkynyl, —C₀₋₆ alkyl-(C₃₋₁₂ carbocycle), -(2- to 6-membered heteroalkyl)-(C₃₋₁₂ carbocycle), —C₀₋₆ alkyl-(3- to 12-membered heterocycle), and -(2- to 6-membered heteroalkyl)-(3- to 12-membered heterocycle) are optionally substituted with one, two, or three R²⁰;
R^{17b} is independently selected at each occurrence from hydrogen, —CN, C₁₋₆ alkyl, C₂₋₆ alkenyl, C₂₋₆ alkynyl, —C₀₋₆ alkyl-(C₃₋₁₂ carbocycle), —C₀₋₆ alkyl-(3- to 12-membered heterocycle), —OR¹², —SR¹², —C(O)OR¹², —OC(O)N(R¹²)(R¹³), —C(O)R¹², —S(O)R¹², —OC(O)R¹², —C(O)N(R¹²)(R¹³), —C(O)C(O)N(R¹²)(R¹³), —S(O)₂R¹², —S(O)(NR¹²)R¹², —S(O)₂N(R¹²)(R¹³), and —S(═O)(═NR¹²)N(R¹²)(R¹³), wherein C₁₋₆ alkyl, C₂₋₆ alkenyl, C₂₋₆ alkynyl, —C₀₋₆ alkyl-(C₃₋₁₂ carbocycle), and —C₀₋₆ alkyl-(3- to 12-membered heterocycle) are optionally substituted with one, two, or three R²⁰;
R¹⁸ is selected from halogen, —CN, C₁₋₆ alkyl, C₂₋₆ alkenyl, C₂₋₆ alkynyl, 2- to 6-membered heteroalkyl, 2- to 6-membered heteroalkenyl, 2- to 6-membered heteroalkynyl, —C₀₋₆ alkyl-(C₃₋₁₂ carbocycle), -(2- to 6-membered heteroalkyl)-(C₃₋₁₂ carbocycle), —C₀₋₆ alkyl-(3- to 12-membered heterocycle), (2- to 6-membered heteroalkyl)-(3- to 12-membered heterocycle), —OR¹², —SR¹², —N(R¹²)(R¹³), ═NR¹², ═C(R¹⁴)₂, —C(O)OR¹², —OC(O)N(R¹²)(R¹³), —N(R¹²)C(O)N(R¹²)(R¹³), —N(R¹²)C(O)OR¹², —N(R¹²)S(O)₂R¹², —C(O)R¹², —S(O)R¹², —OC(O)R¹², —C(O)N(R¹²)(R¹³), —C(O)C(O)N(R¹²)(R¹³), —N(R¹²)C(O)R¹², —S(O)₂R¹², —S(O)(NR¹²)R¹², —S(O)₂N(R¹²)(R¹³), —S(—O)(═NR¹²)N(R¹²)(R¹³), and —OCH₂C(O)OR¹², wherein C₁₋₆ alkyl, C₂₋₆ alkenyl, C₂₋₆ alkynyl, 2- to 6-membered heteroalkyl, 2- to 6-membered heteroalkenyl, 2- to 6-membered heteroalkynyl, —C₀₋₆ alkyl-(C₃₋₁₂ carbocycle), -(2- to 6-membered heteroalkyl)-(C₃₋₁₂ carbocycle), —C₀₋₆ alkyl-(3- to 12-membered heterocycle), and -(2- to 6-membered heteroalkyl)-(3- to 12-membered heterocycle) are optionally substituted with one, two, or three R²⁰;
R¹² is independently selected at each occurrence from hydrogen, C₁₋₆ alkyl, C₂₋₆ alkenyl, C₂₋₆ alkynyl, —C₀₋₆ alkyl-(C₃₋₁₂ carbocycle), and —C₀₋₆ alkyl-(3- to 12-membered heterocycle), wherein C₁₋₆ alkyl, C₂₋₆ alkenyl, C₂₋₆ alkynyl, —C₀₋₆ alkyl-(C₃₋₁₂ carbocycle), and —C₀₋₆ alkyl-(3- to 12-membered heterocycle) are optionally substituted with one, two, or three R²⁰;
R¹³ is independently selected at each occurrence from hydrogen, C₁₋₆ alkyl, and C₁₋₆ haloalkyl; or R¹² and R₁₃ attached to the same nitrogen atom form 3- to 10-membered heterocycle optionally substituted with one, two, or three R²⁰;
R¹⁴ is independently selected at each occurrence from hydrogen, halogen, C₁₋₆ alkyl, C₂₋₆ alkenyl, C₂₋₆ alkynyl, —C₀₋₆ alkyl-(C₃₋₁₂ carbocycle), and —C₀₋₆ alkyl-(3- to 12-membered heterocycle), or two R¹⁴ are taken together with the carbon atom to which they are attached to form C₃₋₁₂ carbocycle or 3- to 12-membered heterocycle, wherein C₁₋₆ alkyl, C₂₋₆ alkenyl, C₂₋₆ alkynyl, —C₀₋₆ alkyl-(C₃₋₁₂ carbocycle), —C₀₋₆ alkyl-(3- to 12-membered heterocycle), C₃₋₁₂ carbocycle, and 3- to 12-membered heterocycle are optionally substituted with one, two, or three R²⁰; and
[symbol] indicates a single or double bond such that all valences are satisfied.

In certain aspects, the present disclosure provides a compound of Formula (II):

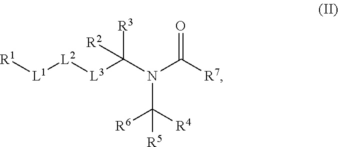

or a pharmaceutically acceptable salt or solvate thereof, wherein:
R¹ is selected from C₃₋₁₂ carbocycle and 3- to 12-membered heterocycle, each of which is optionally substituted with one or more R²⁰;
L¹ is:

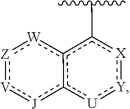

wherein:
W is N, C(R¹⁷), N(R^{17b}), C(R¹⁷)₂, C(O), S(O), or S(O)₂;
Z is N, C(R¹⁷), N(R^{17b}), C(R¹⁷)₂, C(O), S(O), or S(O)₂; wherein W and Z are not both selected from C(O), S(O), and S(O)₂;
V and J are each independently selected from N, C(R¹), C(R¹⁷), N(R¹), N(R^{17b}), C(R¹)(R¹⁷), and C(R¹⁷)₂; wherein exactly one of V and J is C(R¹), N(R¹), or C(R¹)(R¹⁷);
U is N, C(R¹⁷), N(R^{17b}), C(R¹⁷)₂, S(O), S(O)₂, or C(O);
Y is N, C(R¹⁸), N(R^{17b}), C(R¹⁸)(R¹⁷), S(O), S(O)₂, or C(O);
X is N, C(R¹⁷), N(R^{17b}), or C(R¹⁷)₂;
R¹⁷ is independently selected at each occurrence from hydrogen, halogen, —CN, C₁₋₆ alkyl, C₂₋₆ alkenyl, C₂₋₆ alkynyl, 2- to 6-membered heteroalkyl, 2- to 6-membered heteroalkenyl, 2- to 6-membered heteroalkynyl, —C₀₋₆ alkyl-(C₃₋₁₂ carbocycle), -(2- to 6-membered heteroalkyl)-(C₃₋₁₂ carbocycle), —C₀₋₆ alkyl-(3- to 12-membered heterocycle), -(2- to 6-membered heteroalkyl)-(3- to 12-membered heterocycle), —OR¹², —SR¹², —N(R¹²)(R¹³), ═NR¹², —C(R¹⁴)₂, —C(O)OR¹², —OC(O)N(R¹²)(R¹³), —N(R¹²)C(O)N(R¹²)(R¹³), —N(R¹²)C(O)OR¹², —N(R¹²)S(O)₂R¹², —C(O)R¹², —S(O)R¹², —OC(O)R¹², —C(O)N(R¹²)(R¹³), —C(O)C(O)N(R¹²)(R¹³), —N(R¹²)C(O)R¹², —S(O)₂R¹², —S(O)(NR¹²)R¹², —S(O)₂N(R¹²)(R¹³), —S(═O)(═NR¹²)N(R¹²)(R¹³), and —OCH₂C(O)OR¹², wherein C₁₋₆ alkyl, C₂₋₆ alkenyl, C₂₋₆ alkynyl, 2- to 6-membered heteroalkyl, 2- to 6-membered heteroalkenyl, 2- to 6-membered heteroalkynyl, —C₀₋₆ alkyl-(C₃₋₁₂ carbocycle), -(2- to 6-membered heteroalkyl)-(C₃₋₁₂ carbocycle), —C₀₋₆ alkyl-(3- to 12-membered heterocycle), and -(2- to 6-membered heteroalkyl)-(3- to 12-membered heterocycle) are optionally substituted with one, two, or three R²⁰;
R^{17b} is independently selected at each occurrence from hydrogen, —CN, C₁₋₆ alkyl, C₂₋₆ alkenyl, C₂₋₆ alkynyl, —C₀₋₆ alkyl-(C₃₋₁₂ carbocycle), —C₀₋₆ alkyl-(3- to 12-membered heterocycle), —OR¹², —SR¹², —C(O)OR¹², —OC(O)N(R¹²)(R¹³), —C(O)R¹², —S(O)R¹², —OC(O)R¹², —C(O)N(R¹²)(R¹³), —C(O)C(O)N(R¹²)(R¹³), —S(O)₂R¹², —S(O)(NR¹²)R¹², —S(O)₂N(R¹²)(R¹³), and —S(═O)(═NR¹²)N(R¹²)(R¹³), wherein C₁₋₆ alkyl, C₂₋₆ alkenyl, C₂₋₆ alkynyl, —C₀₋₆ alkyl-(C₃₋₁₂ carbocycle), and —C₀₋₆ alkyl-(3- to 12-membered heterocycle) are optionally substituted with one, two, or three R²⁰;
R¹⁸ is selected from halogen, —CN, C₁₋₆ alkyl, C₂₋₆ alkenyl, C₂₋₆ alkynyl, 2- to 6-membered heteroalkyl, 2- to 6-membered heteroalkenyl, 2- to 6-membered heteroalkynyl, —C₀₋₆ alkyl-(C₃₋₁₂ carbocycle), -(2- to 6-membered heteroalkyl)-(C₃₋₁₂ carbocycle), —C₀₋₆ alkyl-(3- to 12-membered heterocycle), -(2- to 6-membered heteroalkyl)-(3- to 12-membered heterocycle), —OR¹², —SR¹², —N(R¹²)(R¹³), ═NR¹², ═C(R¹⁴)₂, —C(O)OR¹², —OC(O)N(R¹²)(R¹³), —N(R¹²)C(O)N(R¹²)(R¹³), —N(R¹²)C(O)OR¹², —N(R¹²)S(O)₂R¹², —C(O)R¹², —S(O)R¹², —OC(O)R¹², —C(O)N(R¹²)(R¹³), —C(O)C(O)N(R¹²)(R¹³), —N(R¹²)C(O)R¹², —S(O)₂R¹², —S(O)(NR¹²)R¹², —S(O)₂N(R¹²)(R¹³), —S(═O)(═NR¹²)N(R¹²)(R¹³), and —OCH₂C(O)OR¹², wherein C₁₋₆ alkyl, C₂₋₆ alkenyl, C₂₋₆ alkynyl, 2- to 6-membered heteroalkyl, 2- to 6-membered heteroalkenyl, 2- to 6-membered heteroalkynyl, —C₀₋₆ alkyl-(C₃₋₁₂ carbocycle), -(2- to 6-membered heteroalkyl)-(C₃₋₁₂ carbocycle), —C₀₋₆ alkyl-(3- to 12-membered heterocycle), and -(2- to 6-membered heteroalkyl)-(3- to 12-membered heterocycle) are optionally substituted with one, two, or three R²⁰;
R¹² is independently selected at each occurrence from hydrogen, C₁₋₆ alkyl, C₂₋₆ alkenyl, C₂₋₆ alkynyl, —C₀₋₆ alkyl-(C₃₋₁₂ carbocycle), and —C₀₋₆ alkyl-(3- to 12-membered heterocycle), wherein C₁₋₆ alkyl, C₂₋₆ alkenyl, C₂₋₆ alkynyl, —C₀₋₆ alkyl-(C₃₋₁₂ carbocycle), and —C₀₋₆ alkyl-(3- to 12-membered heterocycle) are optionally substituted with one, two, or three R²⁰;
R¹³ is independently selected at each occurrence from hydrogen, C₁₋₆ alkyl, and C₁₋₆ haloalkyl; or R¹² and R₁₃ attached to the same nitrogen atom form 3- to 10-membered heterocycle optionally substituted with one, two, or three R²⁰;
R¹⁴ is independently selected at each occurrence from hydrogen, halogen, C₁₋₆ alkyl, C₂₋₆ alkenyl, C₂₋₆ alkynyl, —C₀₋₆ alkyl-(C₃₋₁₂ carbocycle), and —C₀₋₆ alkyl-(3- to 12-membered heterocycle), or two R¹⁴ are taken together with the carbon atom to which they are attached to form C₃₋₁₂ carbocycle or 3- to 12-membered heterocycle, wherein C₁₋₆ alkyl, C₂₋₆ alkenyl, C₂₋₆ alkynyl, —C₀₋₆ alkyl-(C₃₋₁₂ carbocycle), —C₀₋₆ alkyl-(3- to 12-membered heterocycle), C₃₋₁₂ carbocycle, and 3- to 12-membered heterocycle are optionally substituted with one, two, or three R²⁰;
[symbol] indicates a single or double bond such that all valences are satisfied;
L² is selected from a bond, C₁₋₄ alkylene, 2- to 4-membered heteroalkylene, —O—, —N(R¹⁹)—, —C(O)—, —S—, —S(O)₂—, —S(O)—, —P(O)R¹⁹—, —N(R¹⁹)S(O)₂—, —N(R¹⁹)S(O)—, —N(R¹⁹)P(O)R¹⁹—, —S(O)₂N(R¹⁹)—, —S(O)N(R¹⁹)—, —P(O)R¹⁹N(R¹⁹)—, —OS(O)₂—, —OS(O)—, —OP(O)R¹⁹—, —S(O)₂O—, —S(O)O—, and —P(O)R¹⁹O—, wherein C₁₋₄ alkylene and 2- to 4-membered heteroalkylene are optionally substituted with one, two, or three R²⁰;
   L³ and R², together with the atoms to which they are attached, form 3- to 8-membered monocyclic heterocycloalkyl, 7- to 12-membered spirocyclic heterocycloalkyl, or 7- to 12-membered fused bicyclic heterocycloalkyl, each of which is optionally substituted with one, two, or three substituents selected from halogen, —OH, —CN, C₁₋₆ alkyl, C₃₋₆ cycloalkyl, —O(C₁₋₆ alkyl), and —O(C₃₋₆ cycloalkyl), wherein C₁₋₆ alkyl, C₃₋₆ cycloalkyl, —O(C₁₋₆ alkyl), and —O(C₃₋₆ cycloalkyl) are optionally substituted with one, two, or three substituents selected from halogen, —OH, —CN, C₁₋₃ alkyl, C₁₋₃ haloalkyl, C₃₋₆ cycloalkyl, —O(C₁₋₃ alkyl), and —O(C₁₋₃ haloalkyl);
   L³ and R⁶, together with the atoms to which they are attached, form 4- to 8-membered monocyclic heterocycloalkyl, 7- to 12-membered spirocyclic heterocycloalkyl, or 7- to 12-membered fused bicyclic heterocycloalkyl, each of which is optionally substituted with one, two, or three substituents selected from halogen, —OH, —CN, C₁₋₆ alkyl, C₃₋₆ cycloalkyl, —O(C₁₋₆ alkyl), and —O(C₃₋₆ cycloalkyl), wherein C₁₋₆ alkyl, C₃₋₆ cycloalkyl, —O(C₁₋₆ alkyl), and —O(C₃₋₆ cycloalkyl) are optionally substituted with one, two, or three substituents selected from halogen, —OH, —CN, C₁₋₃ alkyl, C₁₋₃ haloalkyl, C₃₋₆ cycloalkyl, —O(C₁₋₃ alkyl), and —O(C₁₋₃ haloalkyl), and wherein the 4- to 8-membered monocyclic heterocycloalkyl formed by L³ and R⁶ is not piperazine; or
   R² and R⁶, together with the atoms to which they are attached, form 3- to 8-membered monocyclic heterocycloalkyl optionally substituted with one, two, or three substituents selected from halogen, —OH, —CN, C₁₋₆ alkyl, C₃₋₆ cycloalkyl, —O(C₁₋₆ alkyl), and —O(C₃₋₆ cycloalkyl), wherein C₁₋₆ alkyl, C₃₋₆ cycloalkyl, —O(C₁₋₆ alkyl), and —O(C₃₋₆ cycloalkyl) are optionally substituted with one, two, or three substituents selected from halogen, —OH, —CN, C₁₋₃ alkyl, C₁₋₃ haloalkyl, C₃₋₆ cycloalkyl, —O(C₁₋₃ alkyl), and —O(C₁₋₃ haloalkyl), and wherein L³ is a bond;
R² is selected from halogen, —CN, C₁₋₆ alkyl, and C₃₋₆ cycloalkyl, or R² and R³, together with the carbon atom to which they are attached, form C₃₋₆ cycloalkyl, wherein C₁₋₆ alkyl and C₃₋₆ cycloalkyl are optionally substituted with one, two, or three substituents selected from halogen, —CN, C₁₋₆ alkyl, C₁₋₆ haloalkyl, C₃₋₆ cycloalkyl, —O(C₁₋₆ alkyl), and —O(C₁₋₆ haloalkyl);
R³ is selected from hydrogen, halogen, —CN, C₁₋₆ alkyl, and C₃₋₆ cycloalkyl, wherein C₁₋₆ alkyl and C₃₋₆ cycloalkyl are optionally substituted with one, two, or three substituents selected from halogen, —CN, C₁₋₆ alkyl, C₁₋₆ haloalkyl, C₃₋₆ cycloalkyl, —O(C₁₋₆ alkyl), and —O(C₁₋₆ haloalkyl);
R⁴, R⁵, and R⁶ are each independently selected from hydrogen, halogen, —CN, C₁₋₆ alkyl, and C₃₋₆ cycloalkyl, or R⁴ and R⁵, together with the carbon atom to which they are attached, form C₃₋₆ cycloalkyl, wherein C₁₋₆ alkyl and C₃₋₆ cycloalkyl are optionally substituted with one, two, or three substituents selected from halogen, —CN, C₁₋₆ alkyl, C₁₋₆ haloalkyl, C₃₋₆ cycloalkyl, —O(C₁₋₆ alkyl), and —O(C₁₋₆ haloalkyl);
R⁷ is selected from

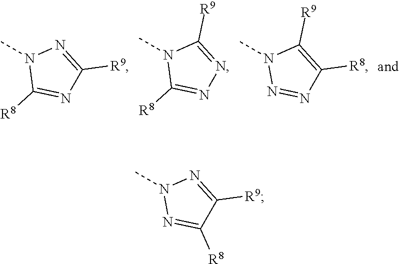

R⁸ and R⁹ are each independently selected from hydrogen, halogen, —CN, C₁₋₆ alkyl, and C₃₋₆ cycloalkyl, wherein C₁₋₆ alkyl and C₃₋₆ cycloalkyl are optionally substituted with one, two, or three substituents selected from halogen, —CN, C₁₋₆ alkyl, —O(C₁₋₆ alkyl), and —O(C₁₋₆ haloalkyl);
R¹⁹ is independently selected at each occurrence from hydrogen, —CN, C₁₋₆ alkyl, C₃₋₆ carbocycle, 3- to 6-membered heterocycle, —OH, —O(C₁₋₆ alkyl), and —O(C₁₋₆ haloalkyl);
R²⁰ is independently selected at each occurrence from halogen, oxo, —CN, C₁₋₆ alkyl, C₂₋₆ alkenyl, C₂₋₆ alkynyl, 2- to 6-membered heteroalkyl, 2- to 6-membered heteroalkenyl, 2- to 6-membered heteroalkynyl, —C₀₋₆ alkyl-(C₃₋₁₂ carbocycle), -(2- to 6-membered heteroalkyl)-(C₃₋₁₂ carbocycle), —C₀₋₆ alkyl-(3- to 12-membered heterocycle), -(2- to 6-membered heteroalkyl)-(3- to 12-membered heterocycle), —OR²², —SR²², —N(R²²)(R²³), ═NR²², ═C(R²¹)₂, —C(O)OR²², —OC(O)N(R²²)(R²³), —N(R²²)C(O)N(R²²)(R²³), —N(R²²)C(O)OR²², —N(R²²)S(O)₂R²², —C(O)R²², —S(O)R²², —OC(O)R²², —C(O)N(R²²)(R²³), —C(O)C(O)N(R²²)(R²³), —N(R²²)C(O)R²², —S(O)₂R²², —S(O)(NR²²)R²², —S(O)₂N(R²²)(R²³)—, —S(═O)(═NR²²)N(R²²)(R²³), and —OCH₂C(O)OR²²; wherein two R²⁰ attached to the same or adjacent atoms optionally join to form C₃₋₁₂ carbocycle or 3- to 12-membered heterocycle; wherein C₁₋₆ alkyl, C₂₋₆ alkenyl, C₂₋₆ alkynyl, 2- to 6-membered heteroalkyl, 2- to 6-membered heteroalkenyl, 2- to 6-membered heteroalkynyl, —C₀₋₆ alkyl-(C₃₋₁₂ carbocycle), -(2- to 6-membered heteroalkyl)-(C₃₋₁₂ carbocycle), —C₀₋₆ alkyl-(3- to 12-membered heterocycle), -(2- to 6-membered heteroalkyl)-(3- to 12-membered heterocycle), C₃₋₁₂ carbocycle, and 3- to 12-membered heterocycle are optionally substituted with one or more substituents independently selected from halogen, oxo, —CN, C₁₋₆ alkyl, C₁₋₆ haloalkyl, C₁₋₆ alkoxy, C₁₋₆ haloalkoxy, —OR²², —SR²², —N(R²²)(R²³), ═NR²², ═C(R²¹)₂, —C(O)OR²², —OC(O)N(R²²)(R²³), —N(R²²)C(O)N(R²²)(R²³), —N(R²²)C(O)OR²², —N(R²²)S(O)₂R²², —C(O)R²², —S(O)R²², —OC(O)R²², —C(O)N(R²²)(R²³), —C(O)C(O)N(R²²)(R²³), —N(R²²)C(O)R²², —S(O)₂R²², —S(O)(NR²²)R²², —S(O)₂N(R²²)(R²³), and —S(═O)(═NR²²)N(R²²)(R²³);
R²¹ is independently selected at each occurrence from hydrogen, halogen, C₁₋₆ alkyl, C₁₋₆ haloalkyl, —C₀₋₆ alkyl-(C₃₋₁₂ carbocycle), and —C₀₋₆ alkyl-(3- to 12-membered heterocycle), or two R²¹ are taken together with the carbon atom to which they are attached to form C₃₋₁₂ carbocycle or 3- to 12-membered heterocycle, each of which is optionally substituted with one, two, or three substituents independently selected from halogen, C₁₋₃ alkyl, C₁₋₃ haloalkyl, and —OH;
R²² is independently selected at each occurrence from hydrogen, C₁₋₆ alkyl, C₁₋₆ haloalkyl, —C₀₋₆ alkyl-(C₃₋₁₂ carbocycle), and —C₀₋₆ alkyl-(3- to 12-membered heterocycle); and
R²³ is independently selected at each occurrence from hydrogen and C₁₋₆ alkyl; or R²² and R²³ attached to the same nitrogen atom form 3- to 10 membered heterocycle.

In some embodiments, W is C(R¹⁷), C(R¹⁷)₂, or C(O); Z is N, C(R¹⁷), N(R^{17b}), or C(R¹⁷)₂; V is C(R¹) or N(R¹); and J is C(R¹⁷) or C(R¹⁷)₂. In some embodiments, W is CH, CH₂, or C(O); Z is N, CCI, N(R^{17b}), or CH₂; V is C(R¹) or N(R¹); and J is CF or CH₂. In some embodiments, W is C(R¹⁷); Z is C(R¹⁷); V is C(R¹); and J is C(R¹⁷). In some embodiments, W is CH; Z is CCI; V is C(R¹); and J is CF. In some embodiments, W is CH; Z is CCF₃; V is C(R¹); and J is CF. In some embodiments, U is N; Y is C(R¹⁸); and X is N. In some embodiments, W is CH; Z is N; V is C(R¹); and J is CF.

In some embodiments, for a compound or modified protein described herein, R¹⁸ is selected from hydrogen, C₁₋₃ alkyl, —OR¹², and 3- to 10-membered heterocycle, wherein C₁₋₃ alkyl and 3- to 10-membered heterocycle are optionally substituted with one, two, or three R²⁰. In some embodiments, L¹ is substituted with C₁₋₃ alkyl, —OR¹², and 3- to 10-membered heterocycle, wherein C₁₋₃ alkyl and 3- to 10-membered heterocycle are optionally substituted with one, two, or three R²⁰. In some embodiments, R¹⁸ is-OR¹². In some embodiments, L¹ is substituted with is-OR¹². In some embodiments, R¹⁸ is-O(C₁₋₃ alkylene) (4- to 10-membered heterocycle), wherein 4- to 10-membered heterocycle is optionally substituted with one, two, or three substituents independently selected from halogen, C₁₋₃ alkyl, C₁₋₃ haloalkyl, and ═C(R²¹)₂, wherein R²¹ is independently selected at each occurrence from hydrogen, halogen, and C₁₋₃ alkyl. In some embodiments, L¹ is substituted with —O(C₁₋₃ alkylene)(4- to 10-membered heterocycle), wherein 4- to 10-membered heterocycle is optionally substituted with one, two, or three substituents independently selected from halogen, C₁₋₃ alkyl, C₁₋₃ haloalkyl, and ═C(R²¹)₂, wherein R²¹ is independently selected at each occurrence from hydrogen, halogen, and C₁₋₃ alkyl.

In some embodiments, for a compound or modified protein described herein, R¹⁸ is selected from hydrogen, C₁₋₆ alkyl, C₂₋₆ alkenyl, C₃₋₁₀ carbocycle, 3- to 10-membered heterocycle, —OR¹², and —N(R¹²)(R¹³), wherein C₁₋₆ alkyl, C₂₋₆ alkenyl, C₃₋₁₀ carbocycle, and 3- to 10-membered heterocycle are optionally substituted with one, two, or three R²⁰. In some embodiments, R¹⁸ is selected from hydrogen, —(C₀₋₃ alkylene)-O—(C₀₋₃ alkylene)-R²⁰, C₁₋₃ alkyl, and 3- to 10-membered heterocycle, wherein each C₀₋₃ alkylene, C₁₋₃ alkyl, and 3- to 10-membered heterocycle are optionally substituted with one, two, or three R²⁰. In some embodiments, R¹⁸ is selected from hydrogen, C₁₋₃ alkyl, —OR¹², and 3- to 10-membered heterocycle, wherein C₁₋₃ alkyl and 3- to 10-membered heterocycle are optionally substituted with one, two, or three R²⁰. In some embodiments, R¹⁸ is OR¹². In some embodiments, R¹⁸ is —O(C₁₋₃ alkylene) (4- to 10-membered heterocycle), wherein 4- to 10-membered heterocycle is optionally substituted with one, two, or three substituents independently selected from halogen, C₁₋₃ alkyl, C₁₋₃ haloalkyl, and —C(R²¹)₂, wherein R²¹ is independently selected at each occurrence from hydrogen, halogen, and C₁₋₃ alkyl.

In some embodiments, for a compound or modified protein described herein, R¹⁸ or a substituent of L¹ is selected from

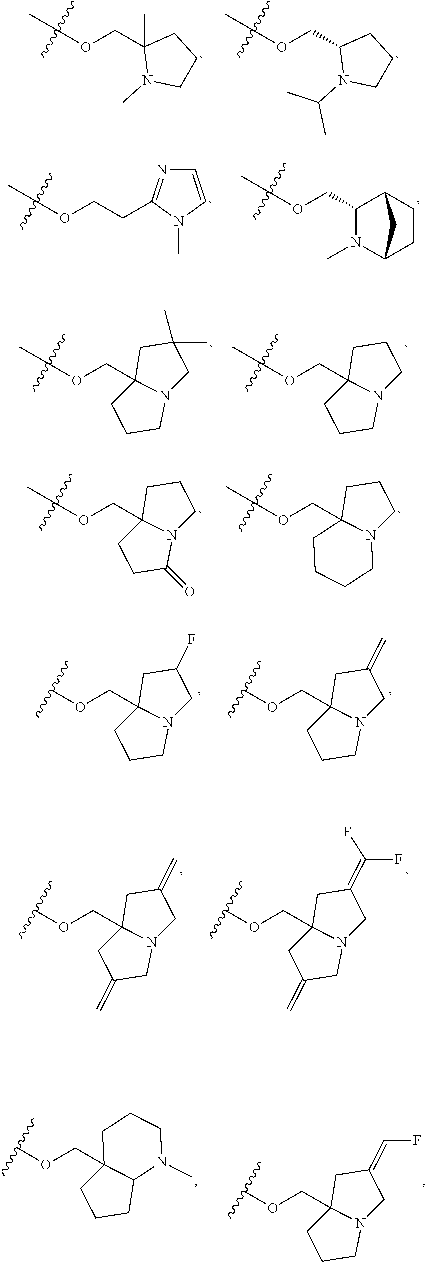

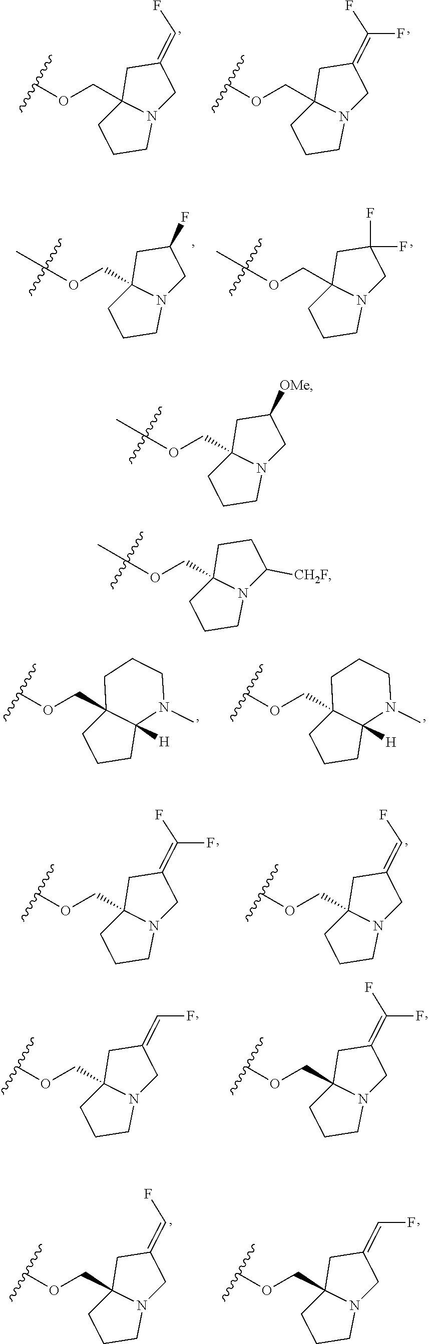

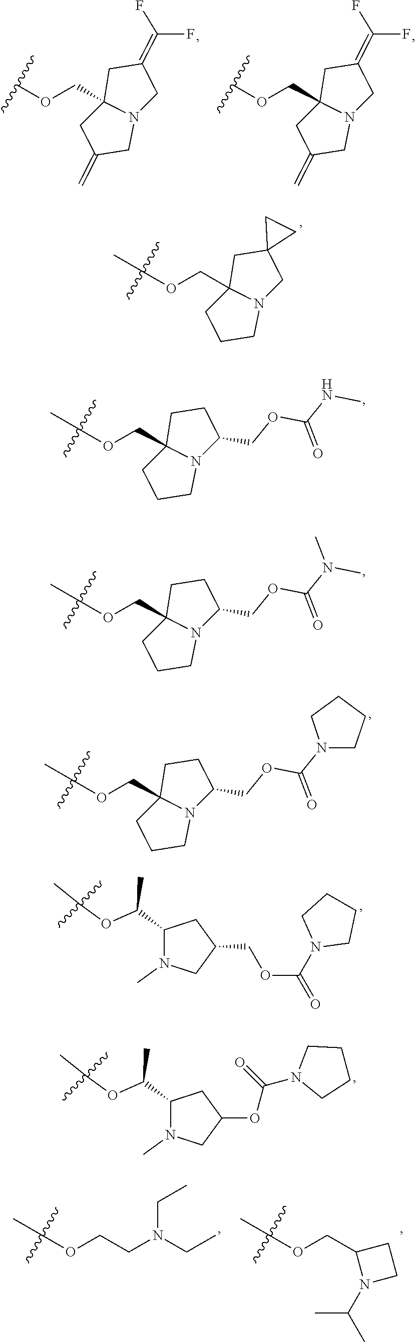

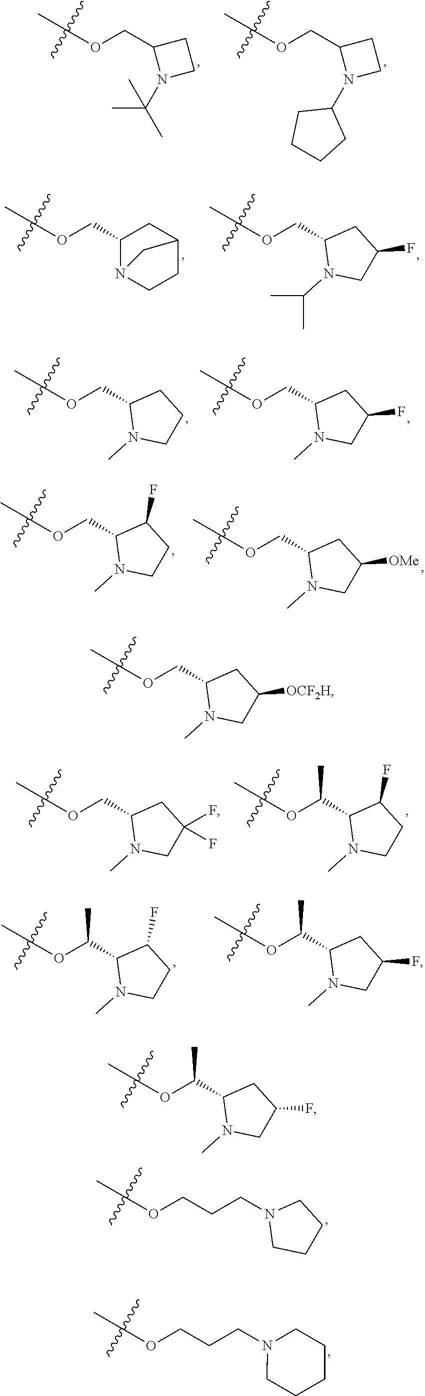

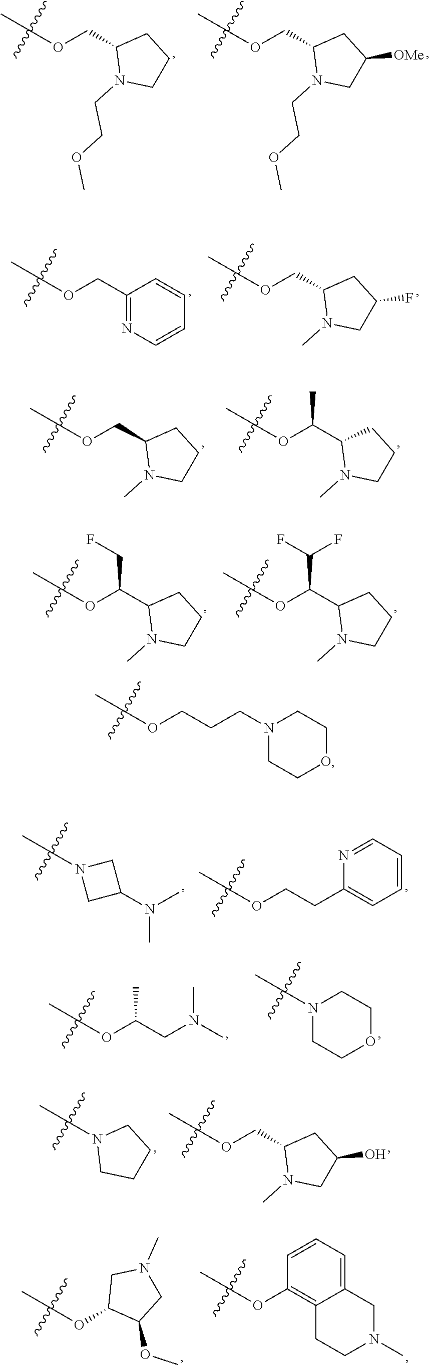

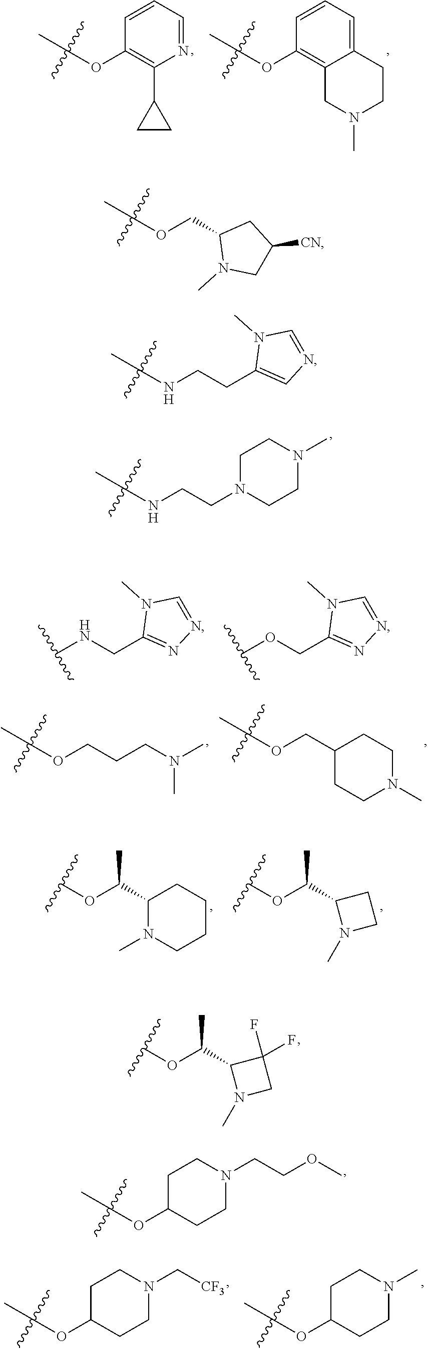

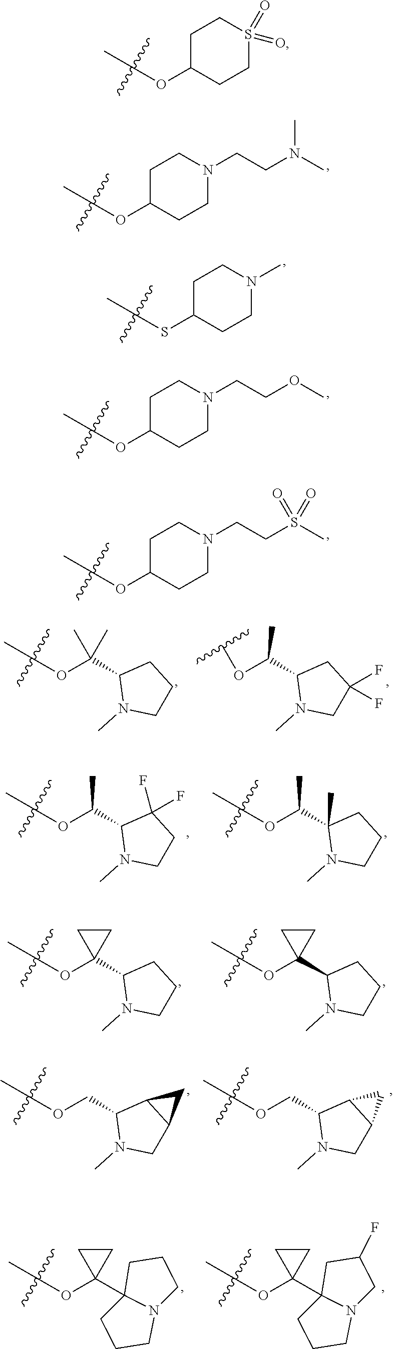

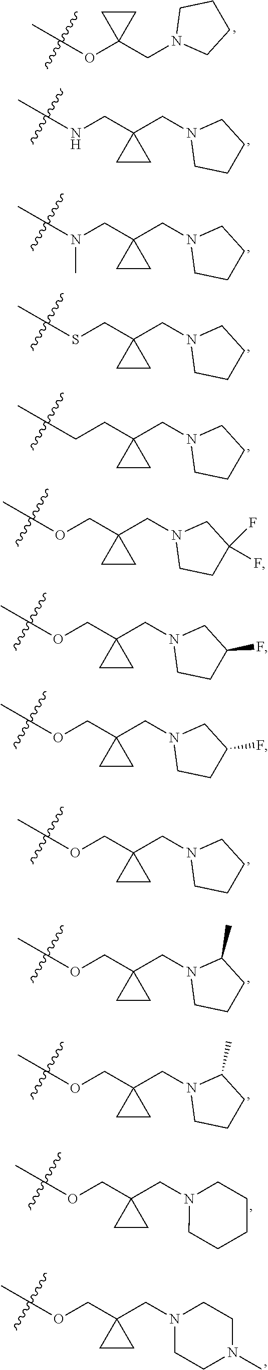

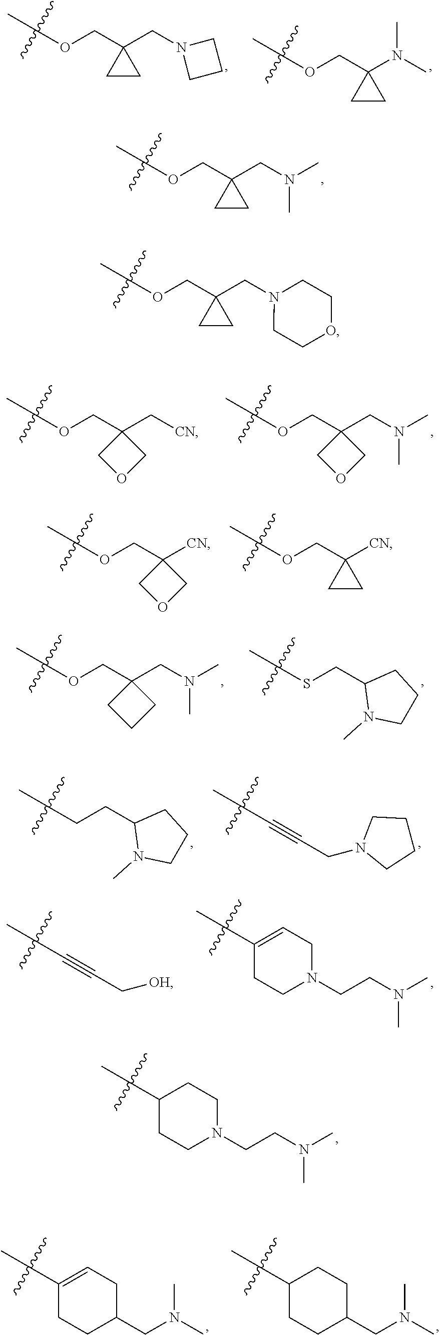

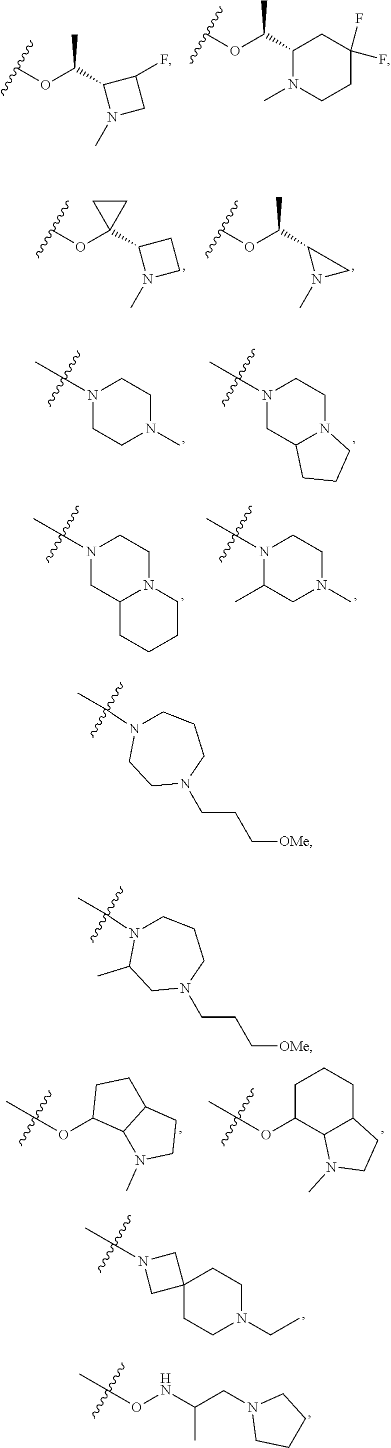

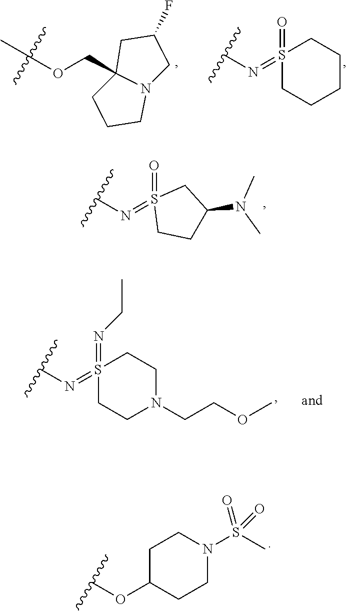

In some embodiments, R¹⁸ or a substituent of L¹ is selected from

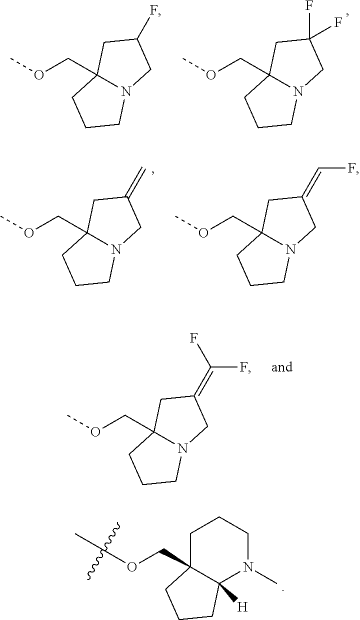

In some embodiments, R¹⁸ or a substituent of L¹ is selected from

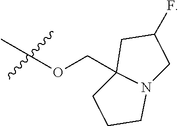 [SMILES: *OCC12CCCN1CC(F)C2]

In some embodiments, R¹⁸ or a substituent of L¹ is selected from

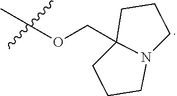 [SMILES: *OCC12CCCN1CCC2]

In some embodiments, R¹⁸ or a substituent of L¹ is selected from

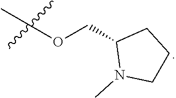 [SMILES: *OC[C@@H]1CCCN1C]

In some embodiments, R¹⁸ or a substituent of L¹ is selected from

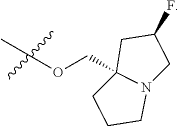 [SMILES: *OC[C@@]12CCCN1C[C@H](F)C2]

In some embodiments, R¹⁸ or a substituent of L¹ is selected from

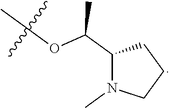 [SMILES: *O[C@@H](C)[C@@H]1CCCN1C]

In some embodiments, R¹⁸ or a substituent of L¹ is selected from

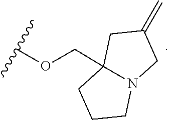 [SMILES: *OCC12CCCN1CC(=C)C2]

In some embodiments, R¹⁸ or a substituent of L¹ is selected from

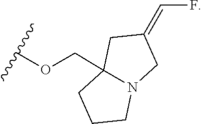 [SMILES: *OCC12CCCN1C/C(=C\F)C2]

In some embodiments, R¹⁸ or a substituent of L¹ is selected from

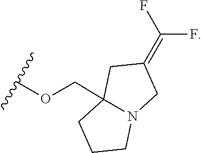 [SMILES: *OCC12CCCN1CC(=C(F)F)C2]

In some embodiments, R¹⁸ or a substituent of L¹ is selected from

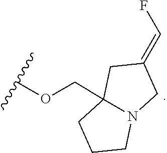 [SMILES: *OCC12CCCN1C/C(=C/F)C2]

In some embodiments, R¹⁸ or a substituent of L¹ is selected from

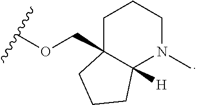 [SMILES: *OC[C@]12CCC[C@H]1N(C)CCC2]

In embodiments, R^{B2} is selected from

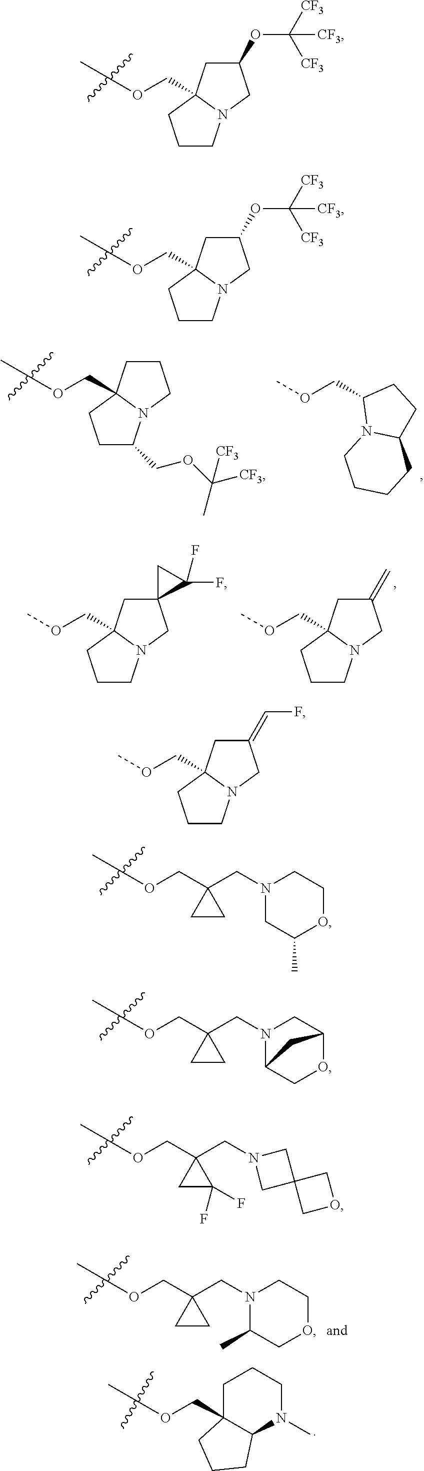

In some embodiments, RB2 is selected from

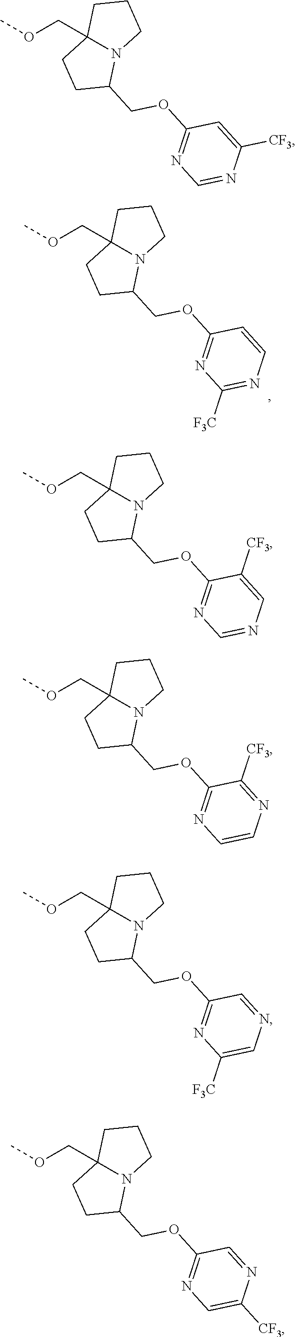

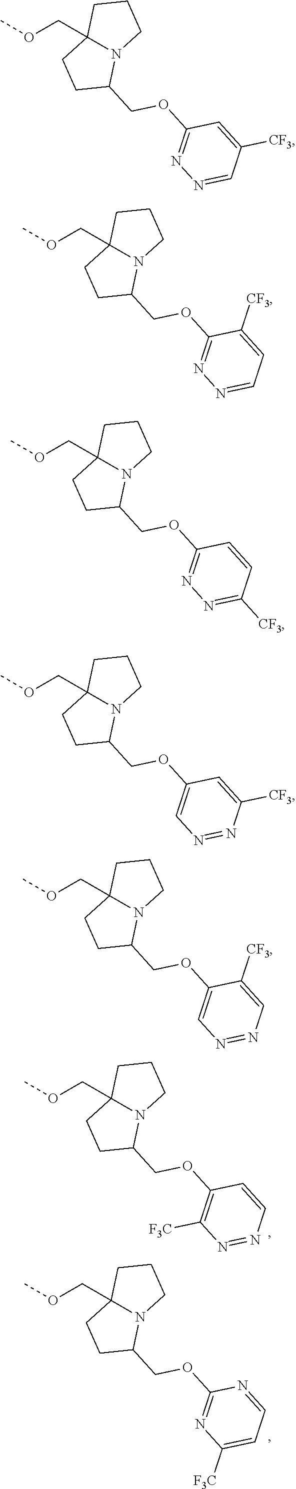

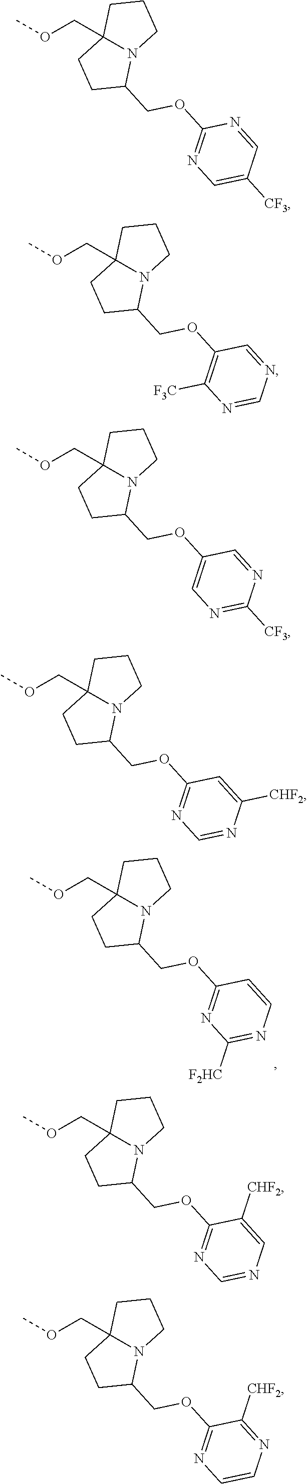

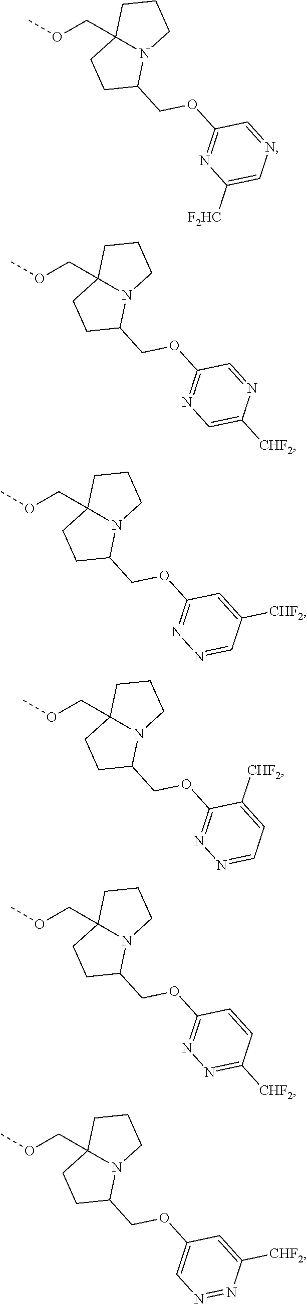

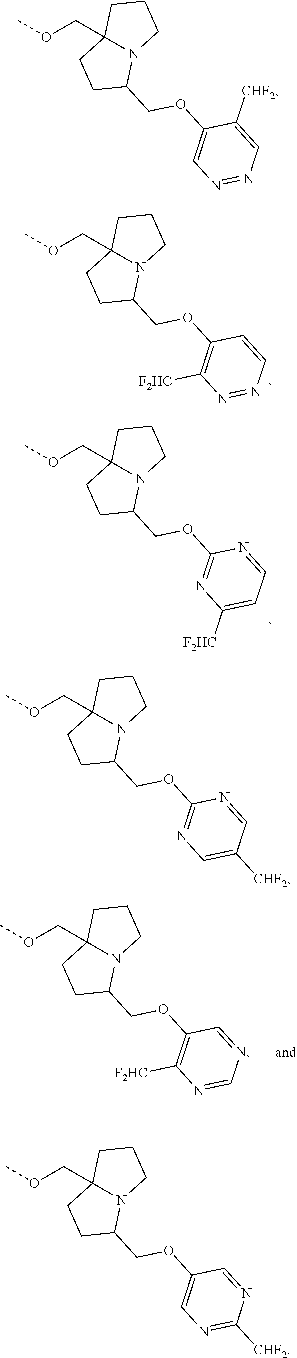

In some embodiments, for a compound of Formula (II), (II-a), (II-b), (II-c), or (II-d) or a modified protein of Formula (I′), (I′-a), (I′-b), (I′-c), (I′-d), (I), (I-a), (I-b), (I-c), or (I-d), L¹ is substituted with one or more halogen, such as F or Cl. In some embodiments, L¹ is substituted with F and Cl. In some embodiments, L¹ is substituted with one R¹⁸ and one or more halogen. In some embodiments, L¹ is substituted with R¹⁸, Cl, and F. In some embodiments, L¹ is substituted with one or more R²⁰, such as three or more R²⁰. In some embodiments, L¹ is substituted with at least one halogen and -(2- to 6-membered heteroalkyl)-(3- to 12-membered heterocycle), wherein the 3- to 12-membered heterocycle is optionally substituted. In some embodiments, L¹ is substituted with at least one halogen and -(2- to 6-membered heteroalkyl)-(3- to 12-membered heterocycle), wherein the 3- to 12-membered heterocycle is substituted with halogen. In some embodiments, L¹ is substituted with Cl, F, and -(2- to 6-membered heteroalkyl)-(3- to 12-membered heterocycle), wherein the 3- to 12-membered heterocycle is substituted with halogen. In some embodiments, L¹ is substituted with Cl, F, and —O(C₁₋₃ alkyl)-(3- to 12-membered heterocycle), wherein the 3- to 12-membered heterocycle is substituted with halogen. In some embodiments, L¹ is substituted with Cl, F, and —O(C₁₋₃ alkyl)-(5- to 9-membered heterocycle), wherein the 5- to 9-membered heterocycle is substituted with halogen. In some embodiments, L¹ is substituted with Cl, F, and —OCH₂(5- to 9-membered heterocycle), wherein the 5- to 9-membered heterocycle is substituted with halogen.

In some embodiments, for a compound of Formula (II), (II-a), (II-b), (II-c), or (II-d) or a modified protein of Formula (I′), (I′-a), (I′-b), (I′-c), (I′-d), (I), (I-a), (I-b), (I-c), or (I-d), R¹-L¹ is

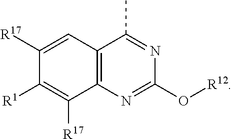

In some embodiments, R¹-L¹ is

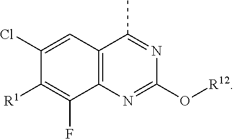

In some embodiments, R¹-L¹ is

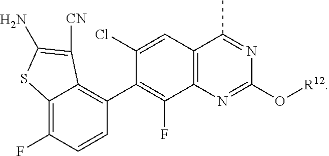

such as

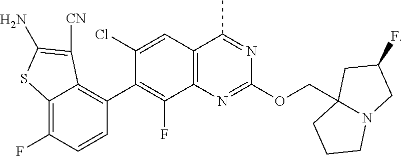

In some embodiments, R¹-L¹ is

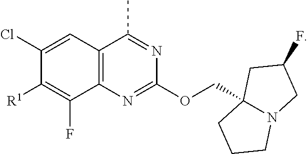

In some embodiments, R¹-L¹ is

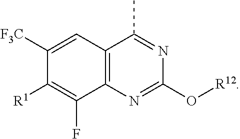

In some embodiments, R¹-L¹ is

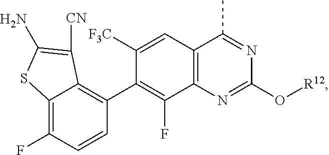

such as

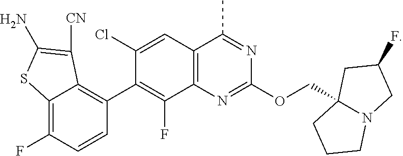

In some embodiments, R¹-L¹ is

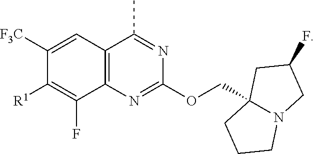

In some embodiments, R¹-L¹ is

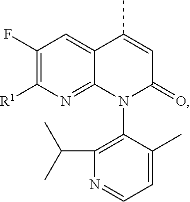

such as

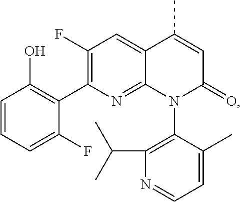

In some embodiments, R¹-L¹ is

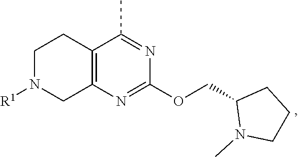

such as

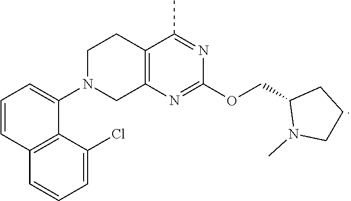

In some embodiments, R¹-L¹ is

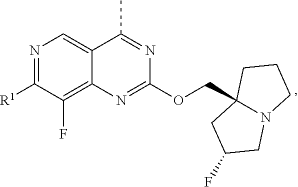

such as

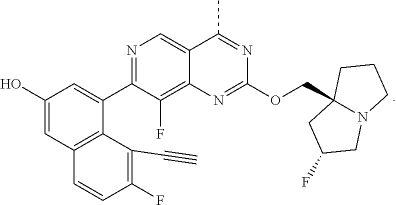

In some embodiments, for a compound of Formula (II), (II-a), (II-b), (II-c), or (II-d) or a modified protein of Formula (I′), (I′-a), (I′-b), (I′-c), (I′-d), (I), (I-a), (I-b), (I-c), or (I-d), R¹-L¹ is selected from

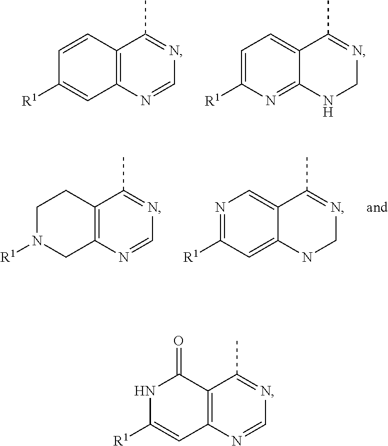

each of which is substituted with one or more R²⁰, such as one, two, three, four, or five R²⁰.

In some embodiments, for a compound of Formula (II), (II-a), (II-b), (II-c), or (II-d) or a modified protein of Formula (I′), (I′-a), (I′-b), (I′-c), (I′-d), (I), (I-a), (I-b), (I-c), or (I-d), L² is selected from a bond, C₁₋₃ alkylene, and 2- to 3-membered heteroalkylene, wherein C₁₋₃ alkylene and 2- to 3-membered heteroalkylene are optionally substituted with one, two, or three R²⁰. In some embodiments, L² is selected from a bond, C₁₋₄ alkylene, 2- to 4-membered heteroalkylene, —N(R¹⁹)—, —C(O)—, —N(R¹⁹)S(O)₂—, and —N(R¹⁹)S(O)—, wherein C₁₋₄ alkylene and 2- to 4-membered heteroalkylene are optionally substituted with one, two, or three R²⁰. In some embodiments, L² is selected from a bond, C₁₋₄ alkylene, 2- to 4-membered heteroalkylene, and —N(R¹⁹)—, wherein C₁₋₄ alkylene and 2- to 4-membered heteroalkylene are optionally substituted with one, two, or three R²⁰. In some embodiments, L² is selected from a bond, C₁₋₃ alkylene, —N(H)C₀₋₃ alkylene-, —N(C₁₋₃ alkyl) C₀₋₃ alkylene-, and —N(C₃₋₆ cycloalkyl) C₀₋₃ alkylene-, wherein C₀₋₃ alkylene, C₁₋₃ alkyl, and C₃₋₆ cycloalkyl are optionally substituted with one, two, or three substituents selected from halogen, C₁₋₃ alkyl, and C₁₋₃ haloalkyl. In some embodiments, L² is selected from a bond, C₁₋₃ alkylene, —N(R¹⁹)—, —N(H)C₁₋₃ alkylene-, —N(C₁₋₃ alkyl) C₁₋₃ alkylene-, and —N(C₃₋₆ cycloalkyl) C₁₋₃ alkylene-, wherein C₁₋₃ alkylene, C₁₋₃ alkyl, and C₃₋₆ cycloalkyl are optionally substituted with one, two, or three substituents selected from halogen, C₁₋₃ alkyl, and C₁₋₃ haloalkyl. In some embodiments, L² is selected from a bond, C₁₋₃ alkylene, 2- to 3-membered heteroalkylene, and —N(R¹⁹)—. In some embodiments, L² is selected from a bond, 2- to 3-membered heteroalkylene, and —N(R¹⁹)—. In some embodiments, L² is selected from —N(H)C₁₋₃ alkylene-, —N(C₁₋₃ alkyl)C₁₋₃ alkylene-, and —N(R¹⁹)—. In some embodiments, L² is selected from —N(R¹⁹)—, —N(CH₃)CH₂—, and —N(CH₃)CH(CH₃)—. In some embodiments, L² is selected from a bond, C₁₋₃ alkylene, —N(H)C₁₋₃ alkylene-, —N(C₁₋₃ alkyl) C₁₋₃ alkylene-, and —N(C₃₋₆ cycloalkyl) C₁₋₃ alkylene-, wherein C₁₋₃ alkylene, C₁₋₃ alkyl, and C₃₋₆ cycloalkyl are optionally substituted with one, two, or three substituents selected from halogen, C₁₋₃ alkyl, and C₁₋₃ haloalkyl. In some embodiments, L² is selected from a bond, —N(CH₃)CH₂—, and —N(CH₃)CH(CH₃)—. In some embodiments, L² is a bond.

In some embodiments, for a compound of Formula (II), (II-b), or (II-c) or a modified protein of Formula (I′), (I′-b), (I′-c), (I), (I-b), or (I-c), R² is selected from C₁₋₆ alkyl and C₃₋₆ cycloalkyl. In some embodiments, R² is selected from —CH₃, —CH₂CH₃, —CH(CH₃)₂, cyclopropyl, 1-methylcyclopropyl, and cyclobutyl.

In some embodiments, for a compound of Formula (II), (II-a), (II-b), (II-c), or (II-d) or a modified protein of Formula (I′), (I′-a), (I′-b), (I′-c), (I′-d), (I), (I-a), (I-b), (I-c), or (I-d), R³ is selected from hydrogen and C₁₋₆ alkyl. In some embodiments, R³ is hydrogen.

In some embodiments, for a compound of Formula (II), (II-a), (II-b), (II-c), or (II-d) or a modified protein of Formula (I′), (I′-a), (I′-b), (I′-c), (I′-d), (I), (I-a), (I-b), (I-c), or (I-d), R⁴, R⁵, and R⁶ are independently selected from hydrogen, C₁₋₃ alkyl, and —(C₁₋₃ alkyl) CN, or R⁴ and R⁵, together with the carbon atom to which they are attached, form C₃₋₆ cycloalkyl.

In some embodiments, for a compound of Formula (II), (II-a), (II-b), (II-c), or (II-d), R⁷ is

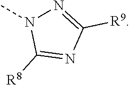

In some embodiments, R⁷ is

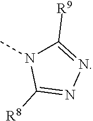

In some embodiments, R⁷ is

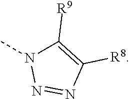

In some embodiments, R⁷ is

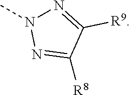

In some embodiments, R⁷ is

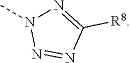

In some embodiments, R⁷ is

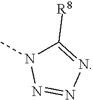

In some embodiments, R⁷ is selected from

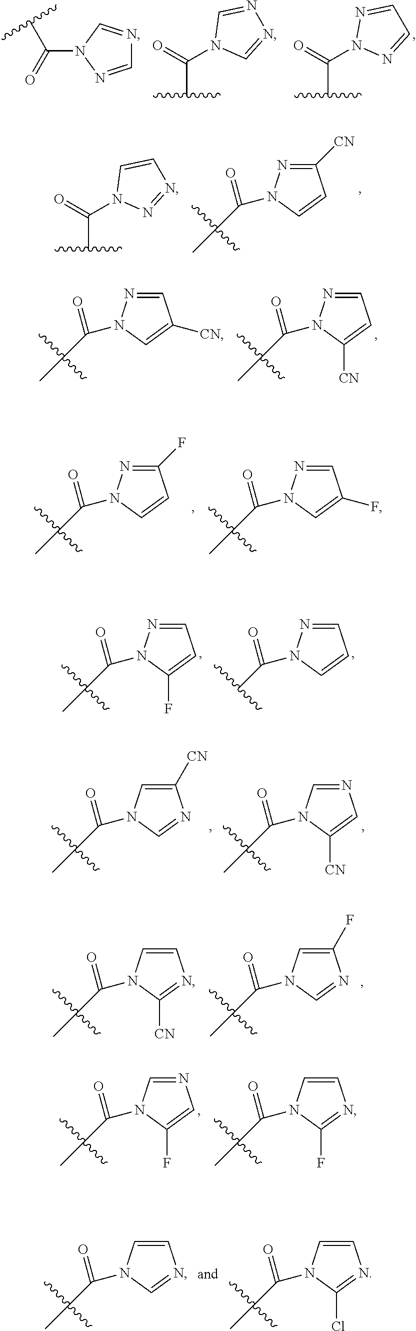

In some embodiments, R⁷ is selected from

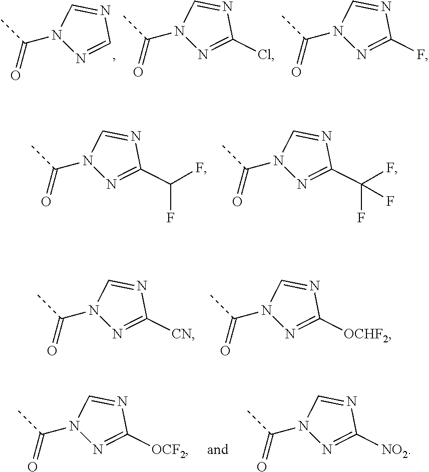

In some embodiments, R⁷ is selected from

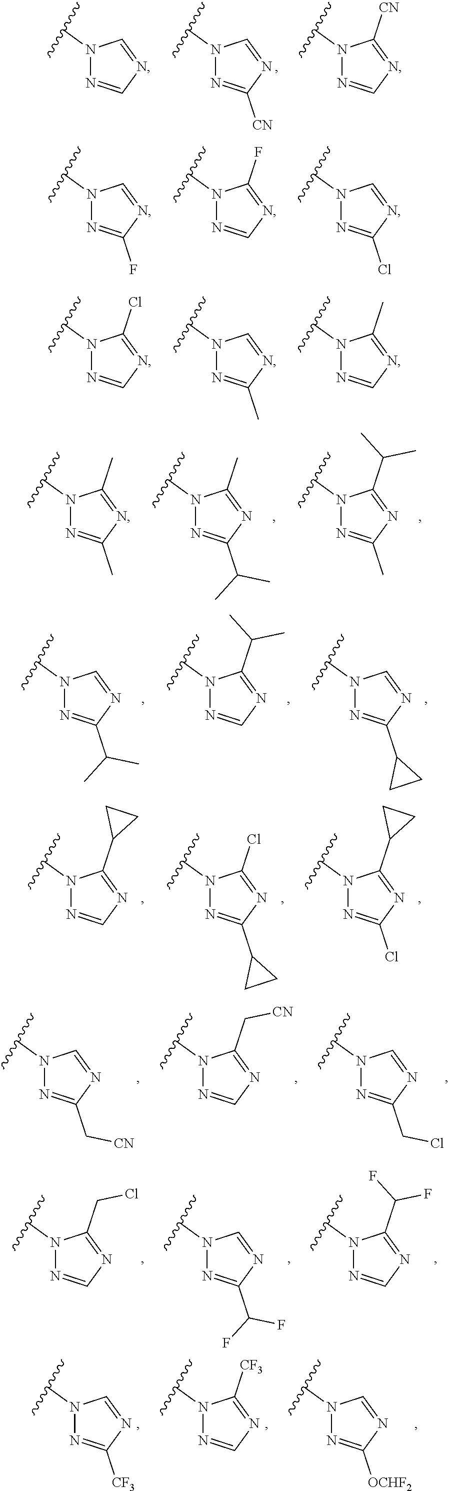

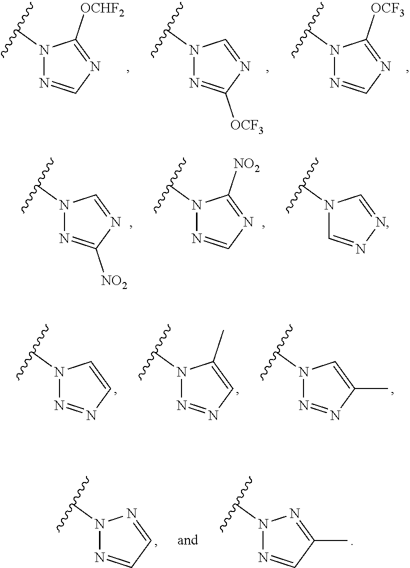

In some embodiments, R⁷ is

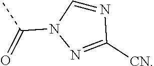

In some embodiments, for a compound of Formula (II), (II-a), (II-b), (II-c), or (II-d), R⁸ is selected from hydrogen, halogen, —CH₃, —CH₂F, —CHF₂, and —CF₃. In some embodiments, R⁸ is hydrogen. In some embodiments, R⁸ is halogen. In some embodiments, R⁸ is —CH₃. In some embodiments, R⁸ is —CH₂F. In some embodiments, R⁸ is —CHF₂. In some embodiments, R⁸ is CF₃. In some embodiments, R⁸ is C₁. In some embodiments, R⁸ is F. In some embodiments, R⁸ is —CN.

In some embodiments, for a compound of Formula (II), (II-a), (II-b), (II-c), or (II-d), R⁹ is selected from hydrogen, halogen, —CH₃, —CH₂F, —CHF₂, and —CF₃. In some embodiments, R⁹ is selected from hydrogen and chloro. In some embodiments, R⁹ is hydrogen. In some embodiments, R⁹ is halogen. In some embodiments, R⁹ is —CH₃. In some embodiments, R⁹ is —CH₂F. In some embodiments, R⁹ is —CHF₂. In some embodiments, R⁹ is CF₃. In some embodiments, R⁹ is Cl . In some embodiments, R⁹ is F. In some embodiments, R⁹ is —CN.

In some embodiments, for a compound of Formula (II), (II-a), (II-b), (II-c), or (II-d) or a modified protein of Formula (I′), (I′-a), (I′-b), (I′-c), (I′-d), (I), (I-a), (I-b), (I-c), or (I-d), R¹ is selected from naphthyl, isoquinolinyl, indazolyl, benzothiazolyl, benzothiophenyl, phenyl, and pyridinyl, each of which is optionally substituted with one or more R²⁰; L¹ is 10-membered bicyclic heterocycle substituted with one, two, three, or four R²⁰; and L² is selected from a bond, C₁₋₃ alkylene, and 2- to 3-membered heteroalkylene, wherein C₁₋₃ alkylene and 2- to 3-membered heteroalkylene are optionally substituted with one, two, or three R²⁰.

In some embodiments, for a compound of Formula (II), (II-a), (II-b), (II-c), or (II-d) or a modified protein of Formula (I′), (I′-a), (I′-b), (I′-c), (I′-d), (I), (I-a), (I-b), (I-c), or (I-d),
R¹-L¹ is

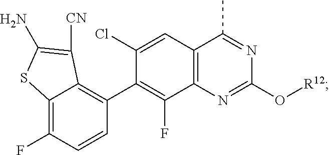

and
L² is selected from a bond, C₁₋₃ alkylene, and 2- to 3-membered heteroalkylene, wherein C₁₋₃ alkylene and 2- to 3-membered heteroalkylene are optionally substituted with one, two, or three R²⁰.

In some embodiments, for a compound of Formula (II), (II-a), (II-b), (II-c), or (II-d) or a modified protein of Formula (I′), (I′-a), (I′-b), (I′-c), (I′-d), (I), (I-a), (I-b), (I-c), or (I-d),
R¹-L¹ is

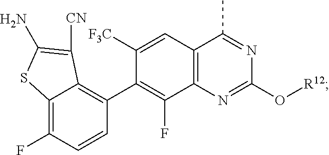

and
L² is selected from a bond, C₁₋₃ alkylene, and 2- to 3-membered heteroalkylene, wherein C₁₋₃ alkylene and 2- to 3-membered heteroalkylene are optionally substituted with one, two, or three R²⁰.

In some embodiments, for a compound of Formula (II), (II-a), (II-b), (II-c), or (II-d),
R¹ is selected from naphthyl, isoquinolinyl, indazolyl, benzothiazolyl, benzothiophenyl, phenyl, and pyridinyl, each of which is optionally substituted with one or more R²⁰;
L¹ is 10-membered bicyclic heterocycle substituted with one, two, three, or four R²⁰;
L² is selected from a bond, C₁₋₃ alkylene, and 2- to 3-membered heteroalkylene, wherein C₁₋₃ alkylene and 2- to 3-membered heteroalkylene are optionally substituted with one, two, or three R²⁰; and
R⁷ is

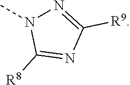

In some embodiments, for a compound of Formula (II), (II-a), (II-b), (II-c), or (II-d),
R¹-L¹ is

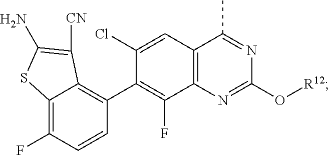

L² is selected from a bond, C₁₋₃ alkylene, and 2- to 3-membered heteroalkylene, wherein C₁₋₃ alkylene and 2- to 3-membered heteroalkylene are optionally substituted with one, two, or three R²⁰; and
R⁷ is

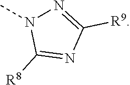

In some embodiments, for a compound of Formula (II), (II-a), (II-b), (II-c), or (II-d),
R¹-L¹ is

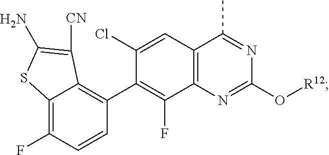

L² is selected from a bond, C₁₋₃ alkylene, and 2- to 3-membered heteroalkylene, wherein C₁₋₃ alkylene and 2- to 3-membered heteroalkylene are optionally substituted with one, two, or three R²⁰; and
R⁷ is

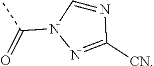

In some embodiments, for a compound of Formula (II), (II-a), (II-b), (II-c), or (II-d),
R¹-L¹ is

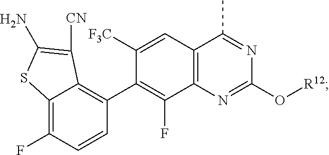

L² is selected from a bond, C₁₋₃ alkylene, and 2- to 3-membered heteroalkylene, wherein C₁₋₃ alkylene and 2- to 3-membered heteroalkylene are optionally substituted with one, two, or three R²⁰; and
R⁷ is

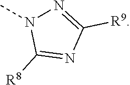

In some embodiments, the compound of Formula (II) is a compound selected from

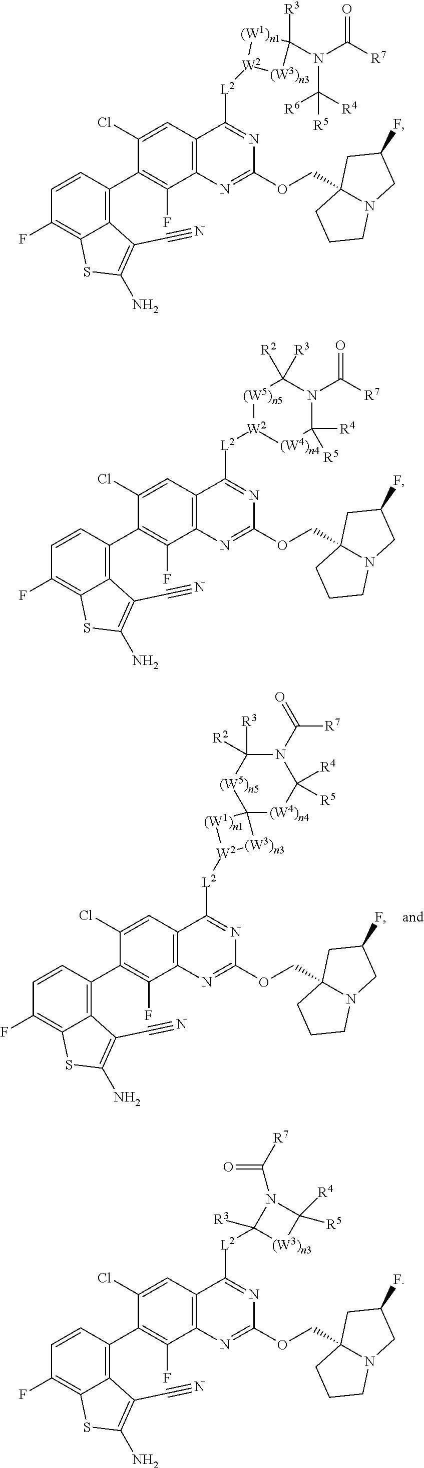

In some embodiments, the compound of Formula (II) is a compound selected from

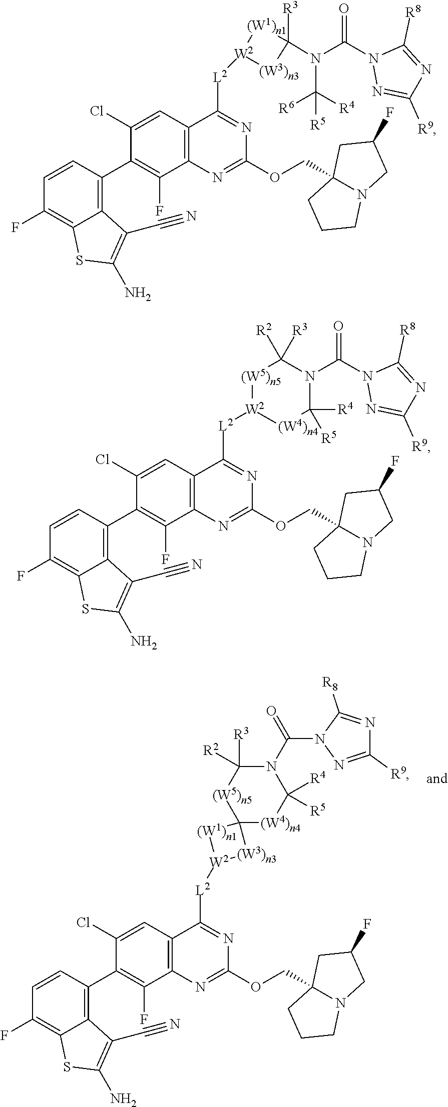

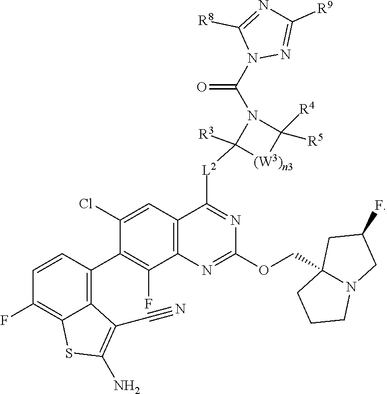

In some embodiments, the compound of Formula (II) is a compound selected from

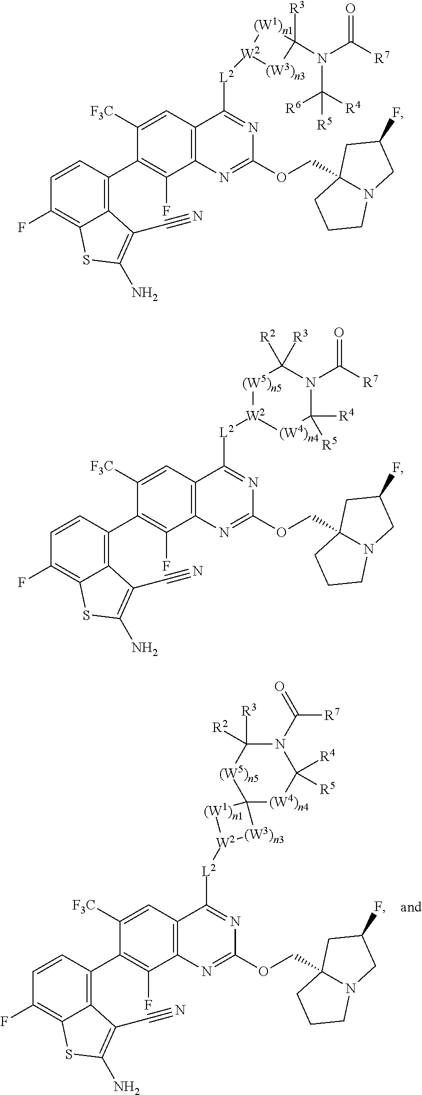

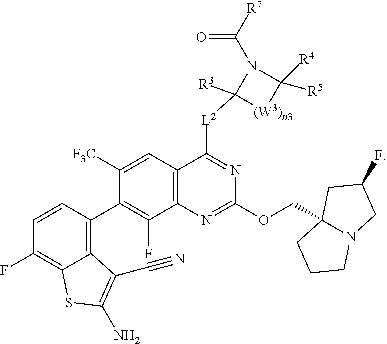

In some embodiments, the compound of Formula (II) is a compound selected from

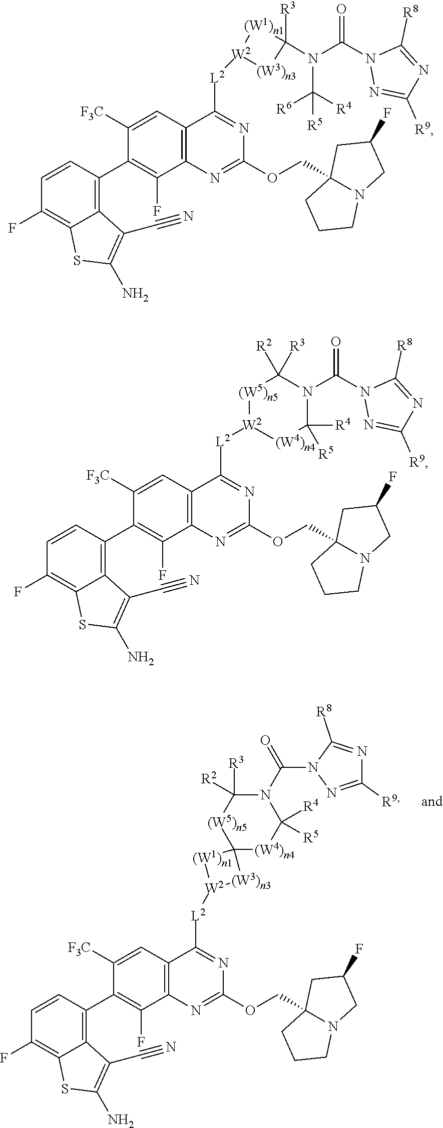

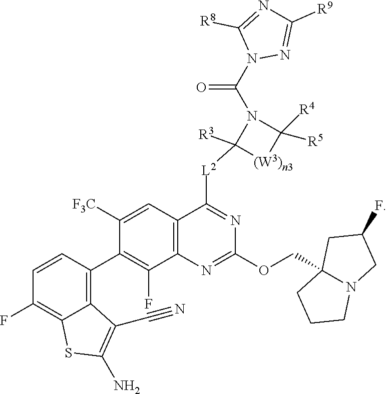

In some embodiments, a compound of Formula (II), (II-a), (II-b), (II-c), or (II-d) reversibly binds to a K-Ras protein when R⁷ is replaced with hydrogen. In some embodiments, the compound reversibly binds to a K-Ras protein with an IC50 of less than 1000 nM, less than 500 nM, less than 250 nM, less than 100 nM, or even less, as assessed by an HTRF assay when R⁷ is replaced with hydrogen. In some embodiments, a compound of Formula (II), (II-a), (II-b), (II-c), or (II-d) reversibly binds to a K-Ras protein when-C(O)R⁷ is replaced with hydrogen. In some embodiments, the compound reversibly binds to a K-Ras protein with an IC₅₀ of less than 1000 nM, less than 500 nM, less than 250 nM, less than 100 nM, or even less, as assessed by an HTRF assay when-C(O)R⁷ is replaced with hydrogen.

In some embodiments, a compound described herein, such as a compound of Formula (II), (II-a), (II-b), (II-c), or (II-d), is provided as a substantially pure stereoisomer. In some embodiments, the stereoisomer is provided in at least 80% enantiomeric excess, such as at least 85%, at least 90%, at least 95%, at least 96%, at least 97%, at least 98%, at least 99%, or at least 99.9% enantiomeric excess.

In some embodiments, a compound disclosed herein, such as a compound of Formula (II), (II-a), (II-b), (II-c), or (II-d), exhibits selective and potent inhibition of K-Ras G12S relative to wildtype K-Ras or other K-Ras mutants (e.g., K-Ras G12V or K-Ras G12D). In some embodiments, a compound disclosed herein exhibits selective and potent inhibition of K-Ras G12S relative to wildtype K-Ras or other K-Ras mutants (e.g., K-Ras G12V or K-Ras G12D), such as a compound of Formula (II), (II-a), (II-b), (II-c), or (II-d), wherein R₇ is a triazole optionally substituted with a methyl, —CH₂—CN, or halogen (e.g., Cl). In some embodiments, a compound disclosed herein exhibits selective and potent inhibition of K-Ras G12S relative to wildtype K-Ras or other K-Ras mutants (e.g., K-Ras G12V or K-Ras G12D), such as a compound of Formula (II), (II-a), (II-b), (II-c), or (II-d), wherein R₇ is a triazole optionally substituted with a methyl, —CH₂—CN, or halogen (e.g., Cl); L¹ is substituted with

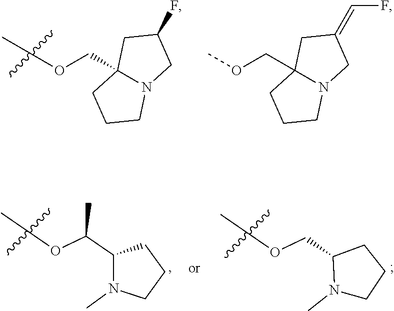

### and R¹ is

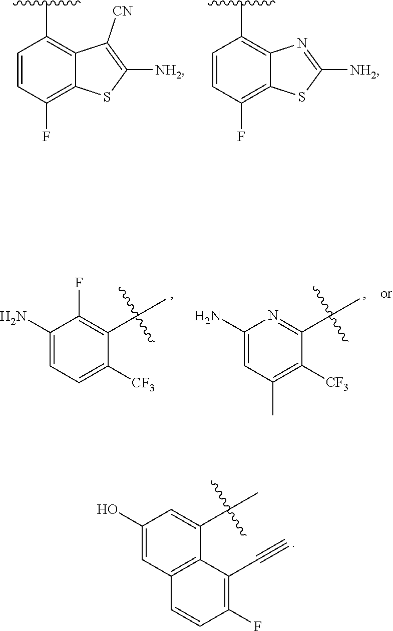 [SMILES: *c1ccc(F)c2sc(N)c(C#N)c12 ; *c1ccc(F)c2sc(N)nc12 ; *c1c(C(F)(F)F)ccc(N)c1F ; *c1nc(N)cc(C)c1C(F)(F)F ; *c1cc(O)cc2ccc(F)c(C#C)c12]

In some embodiments, the K-Ras G12S selective and on-target inhibition is attributed to (1) the warhead (e.g., R⁷ of Formula (II), (II-a), (II-b), (II-c), or (II-d) disclosed herein) being capable of or susceptible to reacting with a serine residue in Ras, such as the serine of SEQ ID No. 4, or the serine corresponding to position 12 of SEQ ID No. 1; and/or (2) the geometry of the atoms linking R⁷ to L¹. These linking atoms may orient the warhead to specifically favor reacting with the serine residue at position 12 of K-Ras G12S mutant.

In some embodiments, a subject warhead exhibits selective engagement of K-Ras G12S relative to K-Ras G12D or wildtype K-Ras by at least 1-fold, and in some instances greater than 2-, 3—, 4-, 5-, 10-, 15-, or 20-fold, or even higher. In some embodiments, a subject warhead exhibits a selective and rapid engagement of K-Ras G12S yielding at least 5%, 10%, 15%, 20%, 25%, 30%, 35%, 40%, 45%, 50%, 55%, 60%, 65%, 70%, 75%, 80%, 85%, 90%, 95%, 96%, 97%, 98%, or 99%, or even higher engagement of G12S within, 10 mins, 20 mins, 30 mins, 1 hr, 2 hrs, 3 hrs, 4 hrs, 5 hrs, 6 hrs, 7 hrs, 8 hrs, 9 hrs, 10 hrs, 20 hrs, or 24 hours. In some embodiments, a selective and rapid engagement of K-Ras G12S is evidenced by at least 50% engagement within 24 hours. In some embodiments, subject compounds specifically engage K-Ras G12S covalently with essentially no detectable labeling of K-Ras G12D when assayed under comparable conditions.

The inclusion of a warhead of the present disclosure may enhance the efficacy or potency of K-Ras G12S inhibition. In some embodiments, a subject compound comprising a subject warhead inhibits K-Ras G12S with higher potency as evidenced by an IC50 value that is at least 10%, 20%, 50%, 100%, 200%, 300%, 400%, or at least 500% lower than the IC50 value of a corresponding control compound that does not comprise the warhead. In some embodiments, a subject compound comprising a subject warhead inhibits K-Ras G12S with higher potency as evidenced by an IC50 value that is at least 1.1-times, 1.2-times, 1.5-times, 2-times, 3-times, 4-times, 5-times, 6-times, 7-times, 8-times, 9-times, 10-times, 15-times, or at least 20-times lower than the IC50 value of a corresponding control compound that does not comprise the warhead, as ascertained in a biochemical assay exemplified in Example 5.

The inclusion of a warhead of the present disclosure may enhance the efficacy or potency with which a subject compound inhibits the proliferation of cells that express a K-Ras G12S mutation and/or a K-Ras G12C mutation. In some embodiments, a subject compound comprising a subject warhead inhibits the proliferation of cells that express a K-Ras G12S mutation and/or a K-Ras G12C mutation with higher potency as evidenced by an IC50 value that is at least 10%, 20%, 50%, 100%, 200%, 300%, 400%, or at least 500% lower than the IC50 value of a corresponding control compound that does not comprise the warhead. In some embodiments, a subject compound comprising a subject warhead inhibits the proliferation of cells that express a K-Ras G12S mutation and/or a K-Ras G12C mutation with higher potency as evidenced by an IC50 value that is at least 1.1-times, 1.2-times, 1.5-times, 2-times, 3-times, 4-times, 5-times, 6-times, 7-times, 8-times, 9-times, 10-times, 15-times, or at least 20-times lower than the IC50 value of a corresponding control compound that does not comprise the warhead, as ascertained in a cellular inhibition assay exemplified in Example 9.

Besides the cellular proliferation inhibitory effect and high potency in reducing K-Ras signaling, particularly signaling mediated by K-Ras G12S mutant and/or a K-Ras G12C mutant, compounds disclosed herein exhibit advantageous ADME and/or DMPK properties. Fine-tuned pharmacological properties are of great significance for improving efficacy and safety of K-Ras inhibitors for therapeutic clinical applications.

In some embodiments, a compound of the present disclosure, such as a compound of Formula (II), (II-a), (II-b), (II-c), or (II-d), exhibits at least one, two, three or more advantageous pharmacological properties. Exemplary superior DMPK properties may include but are not limited to improved metabolic stability, reduced hERG liability, decreased CYP inhibition, increased oral exposure, and decreased serum protein binding (hence increasing the amount of free and available compound circulating in a subject's blood following administration of the compound). In some embodiments, at least one, two, three or more advantageous pharmacological properties are observed in a subject compound having the Formula (II), (II-a), (II-b), (II-c), or (II-d), wherein R₇ is a triazole optionally substituted with a methyl, —CH₂—CN, or halogen (e.g., Cl). In some embodiments, at least one, two, three or more advantageous pharmacological properties (including microsomal stability) are observed in a subject compound having the Formula (II), (II-a), (II-b), (II-c), or (II-d), wherein R₇ is a triazole optionally substituted with a methyl, —CH₂—CN, or halogen (e.g., Cl), wherein L¹ is substituted with

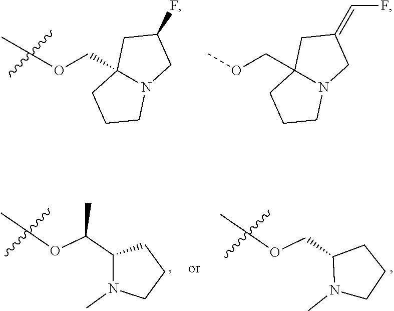

and wherein R¹ is

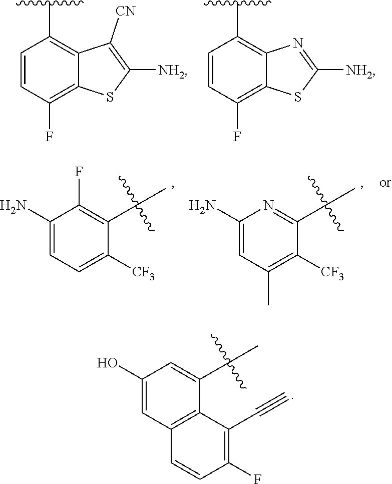

In some embodiments, a subject compound exhibits suitable metabolic stability as ascertained by a T1/2 of mouse liver microsomal metabolism greater than 10 mins, 20 mins, 30 mins, 40 mins, 50 mins, 60 mins or longer as (see Example 11 for experimental procedures). In some embodiments, a subject compound exhibits suitable metabolic stability as ascertained by a T1/2 of human liver microsomal metabolism greater than 10 mins, 20 mins, 30 mins, 40 mins, 50 mins, 60 mins, 100 mins, 120 mins or longer as (see Example 11 for experimental procedures). In yet some other embodiments, a T1/2 of at least 10 mins, 20 mins, 30 mins, 40 mins, 50 mins, 60 mins or longer is observed in both mouse and human microsomal metabolism assays. One or more compounds disclosed herein are expected to exhibit a suitable microsomal stability with a T1/2 greater than 10 mins, 20 mins, 30 mins, 40 mins, 50 mins, 60 mins or longer in mouse and/or human liver microsomal metabolism assays.

In certain aspects, the present disclosure provides a compound selected from

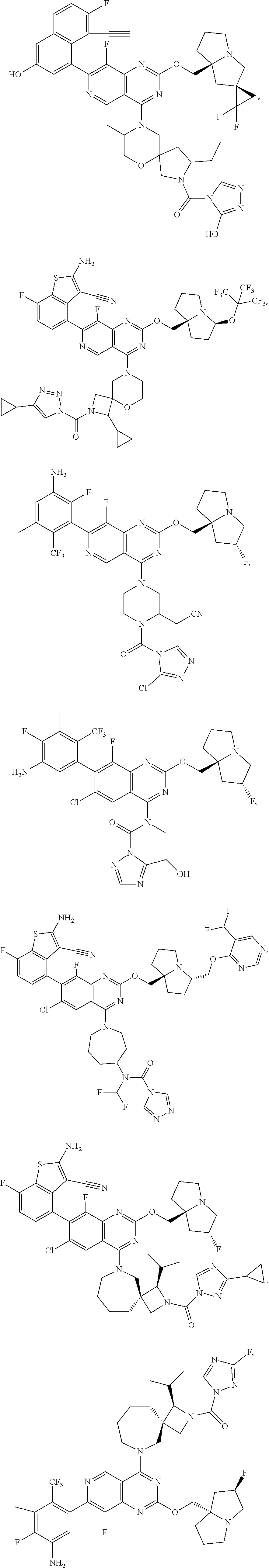

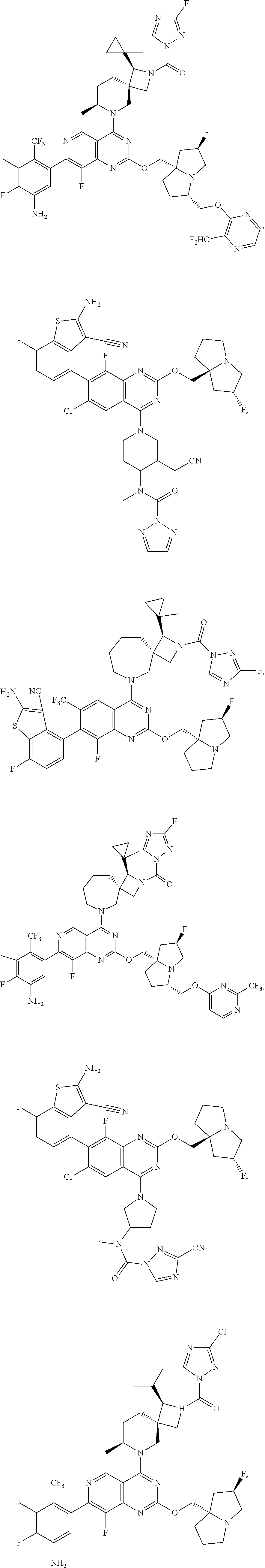

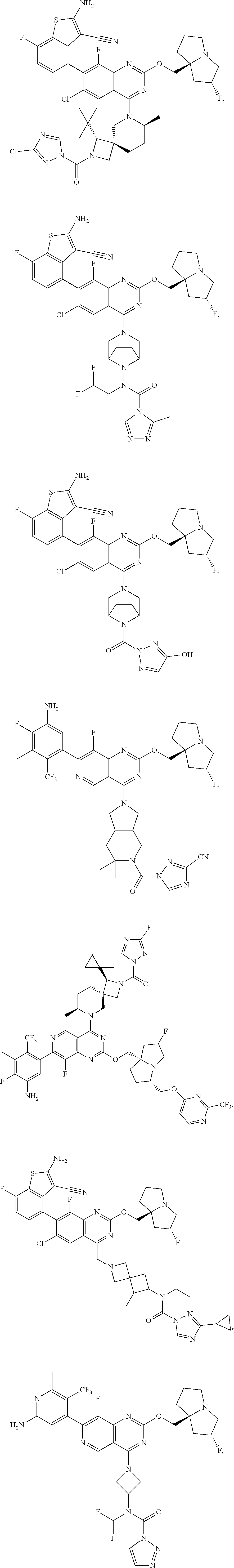

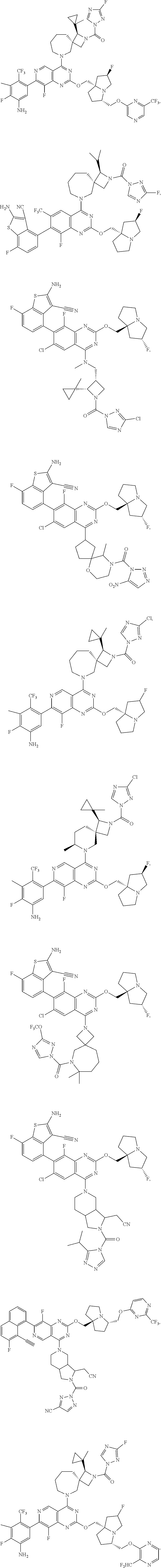

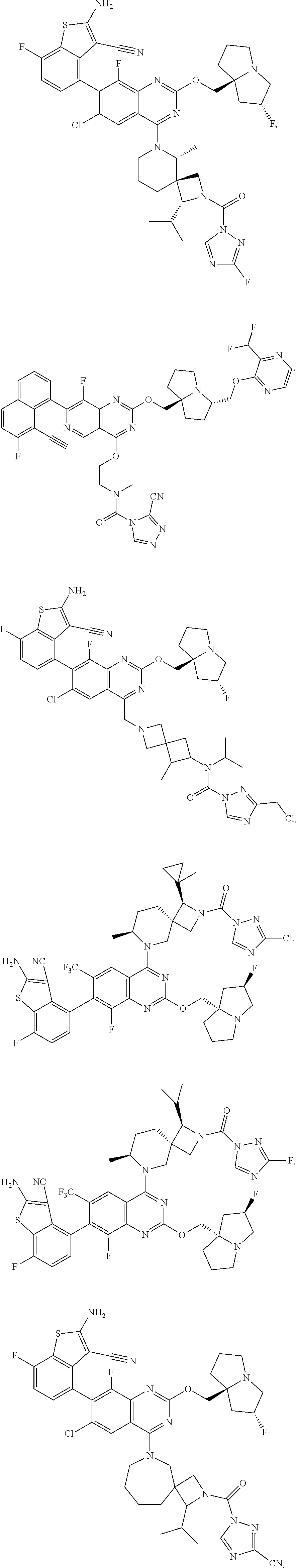

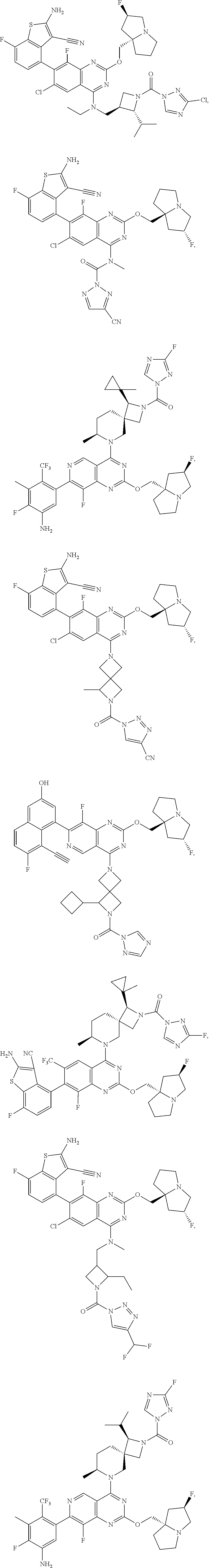

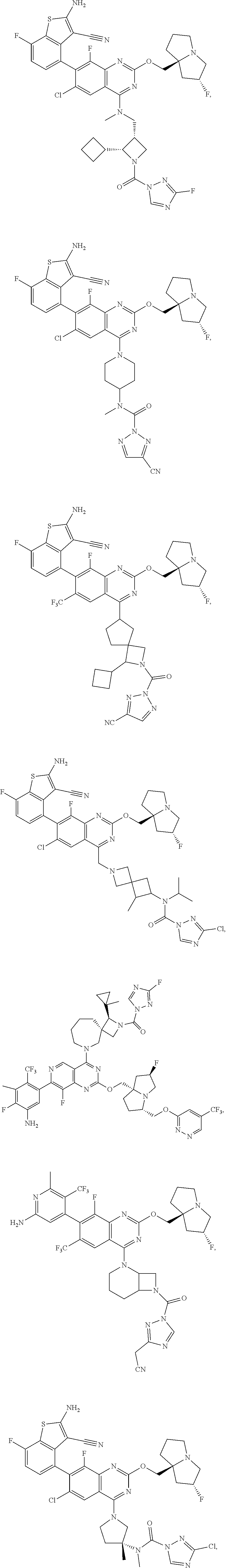

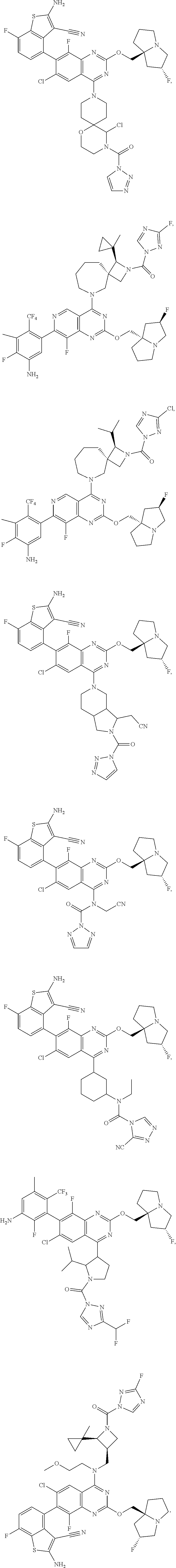

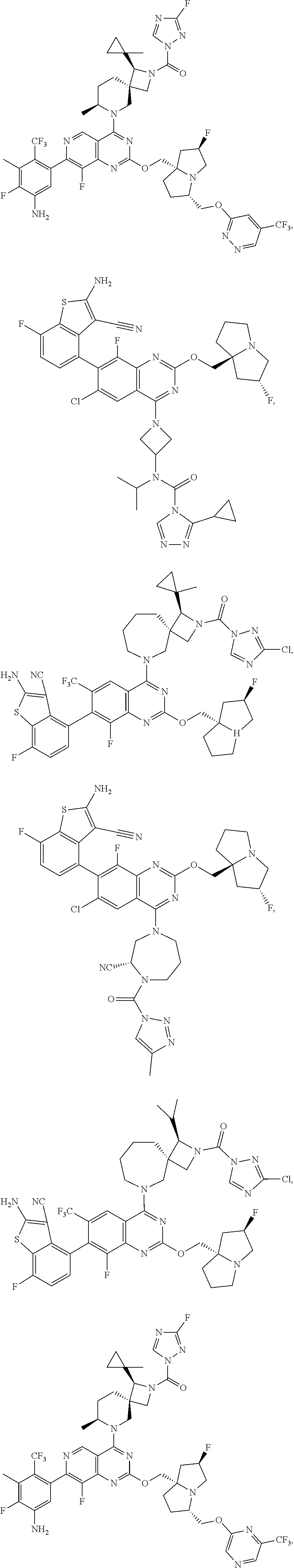

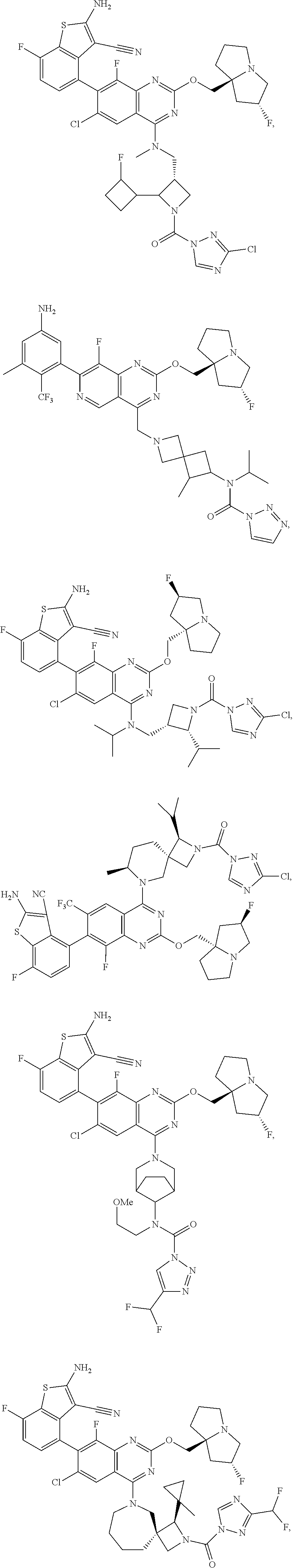

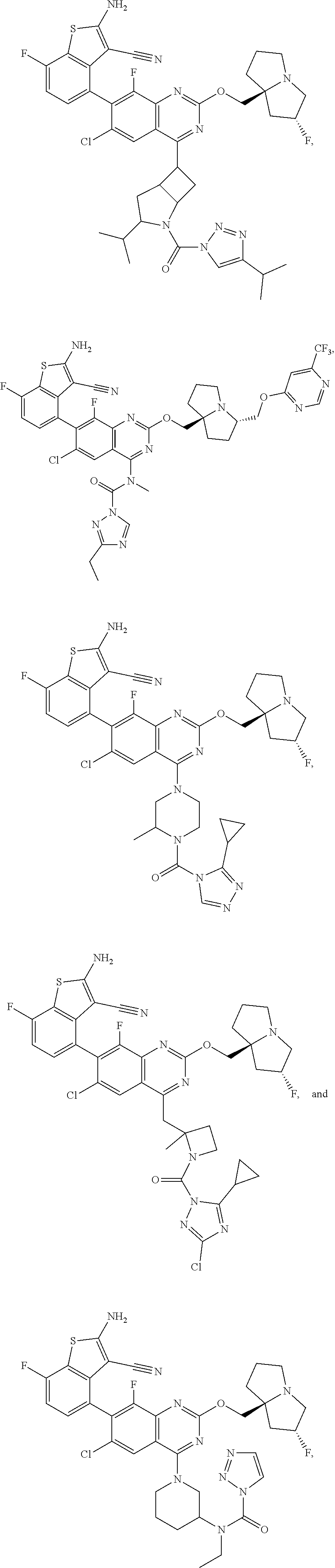

or a pharmaceutically acceptable salt or solvate thereof.

In some embodiments, the present disclosure provides an atropisomer of a compound described herein, such as a compound of Formula (II), (II-a), (II-b), (II-c), or (II-d). In some embodiments, the atropisomer is provided in enantiomeric excess. In some embodiments, the atropisomer is provided in at least 80% enantiomeric excess, such as at least 85%, at least 90%, at least 95%, at least 96%, at least 97%, at least 98%, at least 99%, or at least 99.9% enantiomeric excess. In some embodiments, the compound or modified protein of Formula (I) or (II) is preferably used as a non-racemic mixture, wherein one atropisomer is present in excess of its corresponding enantiomer or epimer. Typically, such mixture contains a mixture of the two isomers in a ratio of at least 9:1, preferably at least 19:1. In some embodiments, the atropisomer is provided in at least 96% enantiomeric excess, meaning the compound has less than 2% of the corresponding enantiomer. In some embodiments, the atropisomer is provided in at least 96% diastereomeric excess, meaning the compound has less than 2% of the corresponding diastereomer.

The term “atropisomers” refers to conformational stereoisomers which occur when rotation about a single bond in the molecule is prevented, restricted, or greatly slowed as a result of steric interactions with other parts of the molecule and wherein the substituents at both ends of the single bond are asymmetrical (i.e., optical activity arises without requiring an asymmetric carbon center or stereocenter). Where the rotational barrier about the single bond is high enough, and interconversion between conformations is slow enough, separation and isolation of the isomeric species may be permitted. Atropisomers are enantiomers (or epimers) without a single asymmetric atom. Atropisomers are typically considered stable if the barrier to interconversion is high enough to permit the atropisomers to undergo little or no interconversion at room temperature for a least a week, preferably at least a year. In some embodiments, an atropisomeric compound of the disclosure does not undergo more than about 5% interconversion to its opposite atropisomer at room temperature during one week when the atropisomeric compound is in substantially pure form, which is generally a solid state. In some embodiments, an atropisomeric compound of the disclosure does not undergo more than about 5% interconversion to its opposite atropisomer at room temperature (approximately 25° C.) during one year. The present chemical entities, pharmaceutical compositions, and methods are meant to include all such possible atropisomers, including racemic mixtures, diastereomeric mixtures, epimeric mixtures, optically pure forms of single atropisomers, and intermediate mixtures.

In some embodiments, the compounds described herein exist as their pharmaceutically acceptable salts. In some embodiments, the methods disclosed herein include methods of treating diseases by administering such pharmaceutically acceptable salts. In some embodiments, the methods disclosed herein include methods of treating diseases by administering such pharmaceutically acceptable salts as pharmaceutical compositions.

In some embodiments, the compounds described herein possess acidic or basic groups and therefore react with any of a number of inorganic or organic bases or inorganic or organic acids to form a pharmaceutically acceptable salt. In some embodiments, such salts are prepared in situ during the final isolation and purification of the compounds described herein, or by separately reacting a purified compound in its free form with a suitable acid or base, and isolating the salt thus formed.

In some embodiments, the compounds described herein exist as solvates. In some embodiments are methods of treating diseases by administering such solvates. Further described herein are methods of treating diseases by administering such solvates as pharmaceutical compositions.

Solvates contain either stoichiometric or non-stoichiometric amounts of a solvent, and, in some embodiments, are formed during the process of crystallization with pharmaceutically acceptable solvents such as water, ethanol, and the like. Hydrates are formed when the solvent is water, or alcoholates are formed when the solvent is alcohol. Solvates of the compounds described herein are conveniently prepared or formed during the processes described herein. By way of example only, hydrates of the compounds described herein are conveniently prepared by recrystallization from an aqueous/organic solvent mixture, using organic solvents including, but not limited to, dioxane, tetrahydrofuran, or MeOH. In addition, the compounds provided herein exist in unsolvated as well as solvated forms. In general, the solvated forms are considered equivalent to the unsolvated forms for the purposes of the compounds and methods provided herein.

In certain aspects, the present disclosure provides a compound of the formula B-L^{BE}-E wherein:
B is a monovalent form of a compound described herein;
L^{BE} is a covalent linker bonded to B and E; and
E is a monovalent form of a degradation enhancer.

A “degradation enhancer” is a compound capable of binding a ubiquitin ligase protein (e.g., E3 ubiquitin ligase protein) or a compound capable of binding a protein that is capable of binding to a ubiquitin ligase protein to form a protein complex capable of conjugating a ubiquitin protein to a target protein. In some embodiments, the degradation enhancer is capable of binding to an E3 ubiquitin ligase protein or a protein complex comprising an E3 ubiquitin ligase protein. In some embodiments, the degradation enhancer is capable of binding to an E2 ubiquitin-conjugating enzyme. In some embodiments, the degradation enhancer is capable of binding to a protein complex comprising an E2 ubiquitin-conjugating enzyme and an E3 ubiquitin ligase protein.

In some embodiments, the degradation enhancer is capable of binding a protein selected from E3A, mdm2, APC, EDD1, SOCS/BC-box/eloBC/CUL5/RING, LNXp80, CBX4, CBLL1, HACE1, HECTD1, HECTD2, HECTD3, HECTD4, HECW1, HECW2, HERC1, HERC2, HERC3, HERC4, HER5, HERC6, HUWE1, ITCH, NEDD4, NEDD4L, PPIL2, PRPF19, PIAS1, PIAS2, PIAS3, PIAS4, RANBP2, RNF4, RBX1, SMURF1, SMURF2, STUB1, TOPORS, TRIP12, UBE3A, UBE3B, UBE3C, UBE3D, UBE4A, UBE4B, UBOX5, UBR5, VHL (von-Hippel-Lindau ubiquitin ligase), WWP1, WWP2, Parkin, MKRN1, CMA (chaperon-mediated autophage), SCFb-TRCP (Skip-Cullin-F box (Beta-TRCP) ubiquitin complex), b-TRCP (b-transducing repeat-containing protein), cIAP1 (cellular inhibitor of apoptosis protein 1), APC/C (anaphase-promoting complex/cyclosome), CRBN (cereblon), CULA-RBX1-DDB1-CRBN(CRLACRBN) ubiquitin ligase, XIAP, IAP, KEAP1, DCAF15, RNF114, DCAF16, AhR, SOCS2, KLHL12, UBR2, SPOP, KLHL3, KLHL20, KLHDC2, SPSB1, SPSB2, SPSB4, SOCS6, FBXO4, FBXO31, BTRC, FBW7, CDC20, PML, TRIM21, TRIM24, TRIM33, GID4, avadomide, iberdomide, and CC-885. In some embodiments, the degradation enhancer is capable of binding a protein selected from UBE2A, UBE2B, UBE2C, UBE2D1, UBE2D2, UBE2D3, UBE2DR, UBE2E1, UBE2E2, UBE2E3, UBE2F, UBE2G1, UBE2G2, UBE2H, UBE2I, UBE2J1, UBE2J2, UBE2K, UBE2L3, UBE2L6, UBE2L1, UBE2L2, UBE2L4, UBE2M, UBE2N, UBE20, UBE2Q1, UBE2Q2, UBE2R1, UBE2R2, UBE2S, UBE2T, UBE2U, UBE2V1, UBE2V2, UBE2W, UBE2Z, ATG3, BIRC6, and UFC1. In some embodiments, the degradation enhancer is a compound described in Ishida and Ciulli, SLAS Discovery 2021, Vol. 25 (4) 484-502, which is incorporated by reference in its entirety for any purpose, for example VH032, VH101, VH298, thalidomide, bestatin, methyl bestatin, nutlin, idasanutlin, bardoxolone, bardoxolone methyl, indisulam

(E7070), E7820, chloroquinoxaline sulfonamide (CQS), nimbolide, KB02, ASTX660, lenalidomide, or pomalidomide.

In some embodiments, the degradation enhancer is a compound described in US20180050021, WO2016146985, WO2018189554, WO2018119441, WO2018140809, WO2018119448, WO2018119357, WO2018118598, WO2018102067, WO201898280, WO201889736, WO201881530, WO201871606, WO201864589, WO201852949, WO2017223452, WO2017204445, WO2017197055, WO2017197046, WO2017180417, WO2017176958, WO201711371, WO2018226542, WO2018223909, WO2018189554, WO2016169989, WO2016146985, CN105085620B, CN106543185B, U.S. Pat. Nos. 10,040,804, 9,938,302, 10,144,745, 10,145,848, 9,938,264, 9,632,089, 9,821,068, 9,758,522, 9,500,653, 9,765,019, 8,507,488, 8,299,057, US20180298027, US20180215731, US20170065719, US20170037004, US20160272639, US20150291562, or US20140356322, each of which is incorporated by reference in its entirety for any purpose.

In some embodiments, L^{BE} is -L^{BE1}-L^{BE2}-L^{BE3}-L^{BE4}L^{BE5}-;
L^{BE1}, L^{BE2}, L^{BE3}, L^{BE4}, and L^{BE5} are independently a bond, —O—, —N(R¹²)—, —C(O)—, —N(R¹²)C(O)—, —C(O)N(R¹²)—, —S—, —S(O)₂—, —S(O)—, —S(O)₂N(R¹²)—, —S(O)N(R¹²)—, —N(R¹²)S(O)—, —N(R¹²)S(O)₂—, C₁₋₆ alkylene, (—O—C₁₋₆ alkyl)ₓ-, (—C₁₋₆ alkyl-O)ₓ—, C₂₋₆ alkenylene, C₂₋₆ alkynylene, C₁₋₆ haloalkylene, C₃₋₁₂ cycloalkylene, C₁₋₁₁ heterocycloalkylene, C₆₋₁₂ arylene, or C₁₋₁₁ heteroarylene, wherein C₁₋₆ alkylene, C₂₋₆ alkenylene, C₂₋₆ alkynylene, C₁₋₆ haloalkylene, C₃₋₁₂ cycloalkylene, C₁₋₁₁ heterocycloalkylene, C₆₋₁₂ arylene, or C₁₋₁₁ heteroarylene are optionally substituted with one, two, or three R²⁰; and wherein each C₁₋₆ alkyl of (—O—C₁₋₆ alkyl)_{z}- and (—C₁₋₆ alkyl-O)_{z}— is optionally substituted with one, two, or three R²⁰; and
z is independently an integer from 0 to 10.

In some embodiments, L^{BE} is —(O—C₂ alkyl)_{z}- and z is an integer from 1 to 10. In some embodiments, L^{BE} is —(C₂ alkyl-O—)_{z}- and z is an integer from 1 to 10. In some embodiments, L^{BE} is —(CH₂)_{zz1}L^{BE2}(CH₂O)_{zz2}—, wherein L^{BE2} is a bond, a 5- or 6-membered heterocyclene, phenylene, —C₂₋₄ alkynylene, —SO₂— or —NH—; and zz1 and zz2 are independently an integer from 0 to 10. In some embodiments, L^{BE} is —(CH₂)_{zz1}(CH₂O)_{zz2}—, wherein zz1 and zz2 are each independently an integer from 0 to 10. In some embodiments, L^{BE} is a PEG linker (e.g., divalent linker of 1 to 10 ethylene glycol subunits). In some embodiments, E is a monovalent form of a compound selected from

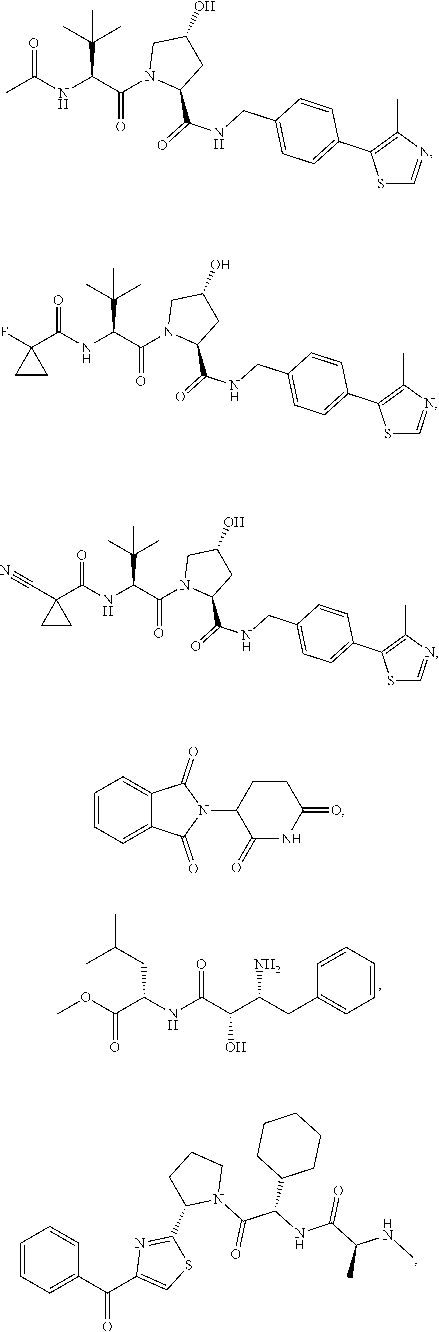 [SMILES: CC(=O)N[C@H](C(=O)N1C[C@H](O)C[C@H]1C(=O)NCc1ccc(-c2scnc2C)cc1)C(C)(C)C ; Cc1ncsc1-c1ccc(CNC(=O)[C@@H]2C[C@@H](O)CN2C(=O)[C@@H](NC(=O)C2(F)CC2)C(C)(C)C)cc1 ; Cc1ncsc1-c1ccc(CNC(=O)[C@@H]2C[C@@H](O)CN2C(=O)[C@@H](NC(=O)C2(C#N)CC2)C(C)(C)C)cc1 ; O=C1CCC(N2C(=O)c3ccccc3C2=O)C(=O)N1 ; COC(=O)[C@H](CC(C)C)NC(=O)[C@@H](O)[C@H](N)Cc1ccccc1 ; CN[C@@H](C)C(=O)N[C@H](C(=O)N1CCC[C@H]1c1nc(C(=O)c2ccccc2)cs1)C1CCCCC1]

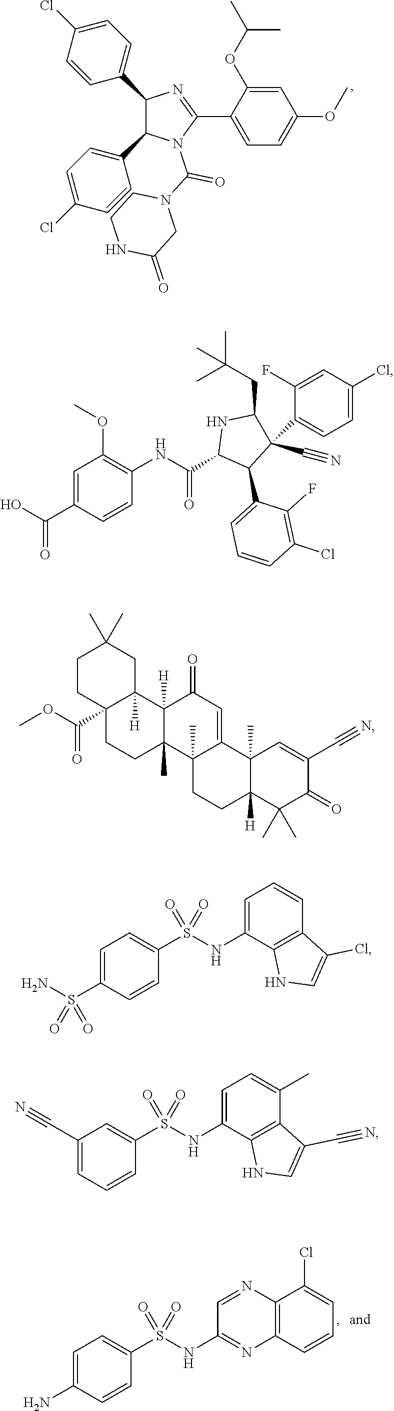

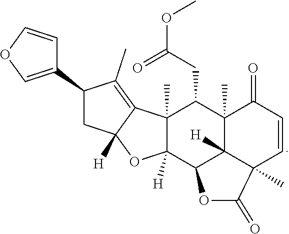

In embodiments, E is a monovalent form of a compound selected from

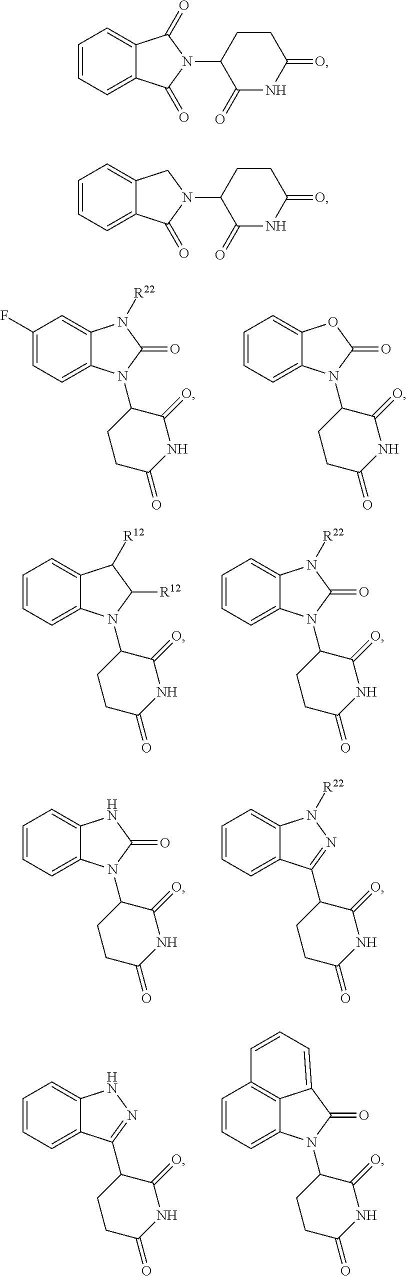

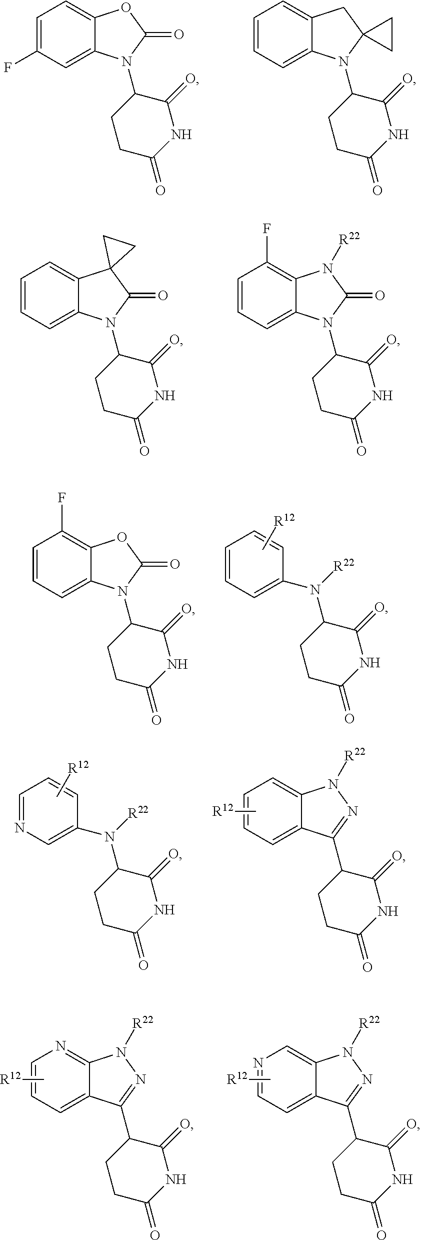

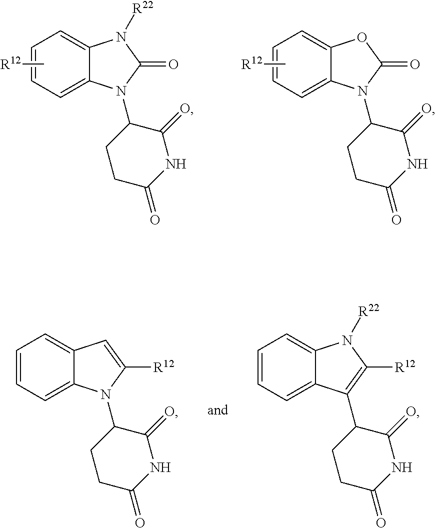

The chemical entities described herein can be synthesized according to one or more illustrative schemes herein and/or techniques known in the art. Materials used herein are either commercially available or prepared by synthetic methods generally known in the art. These schemes are not limited to the compounds listed in the examples or by any particular substituents, which are employed for illustrative purposes. Although various steps are described and depicted in Schemes 1 and 2, the steps in some cases may be performed in a different order than the order shown in Schemes 1 and 2. Various modifications to these synthetic reaction schemes may be made and will be suggested to one skilled in the art having referred to the present disclosure. Numberings or R groups in each scheme typically have the same meanings as those defined elsewhere herein unless otherwise indicated.

Unless specified to the contrary, the reactions described herein take place at atmospheric pressure, generally within a temperature range from −10° C. to 200° C. Further, except as otherwise specified, reaction times and conditions are intended to be approximate, e.g., taking place at about atmospheric pressure within a temperature range of about −10° C. to about 110° C. over a period of about 1 to about 24 hours; reactions left to run overnight average a period of about 16 hours.

In general, compounds of the disclosure may be prepared by the following reaction schemes:

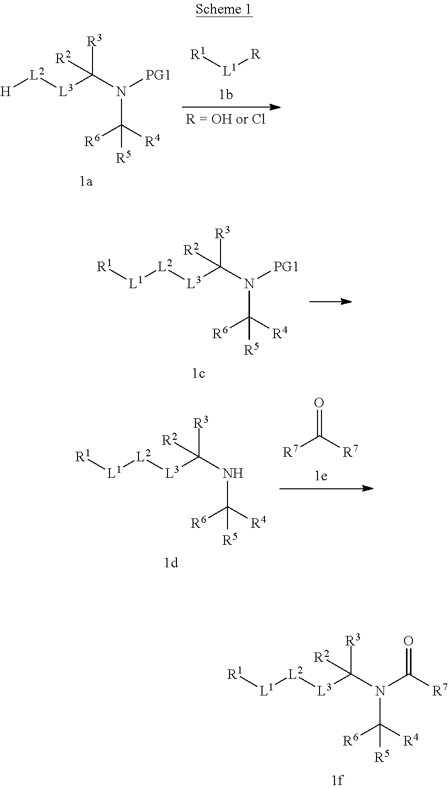

In some embodiments, a compound of Formula 1f may be prepared according to Scheme 1. For example, an amination reaction (e.g., using PyBOP and DBU) can be utilized to form a C—N bond between a suitably-protected amine (e.g., PG1 is Boc, Bus, Cbz, or Fmoc) of 1a and an aryl or heteroaryl alcohol or chloride of 1b to provide 1c. Removal of the N-protecting group to reveal secondary amine 1d can be followed by reaction with compound 1e in the presence of a suitable base, such as DIPEA, to provide a compound of Formula 1f.

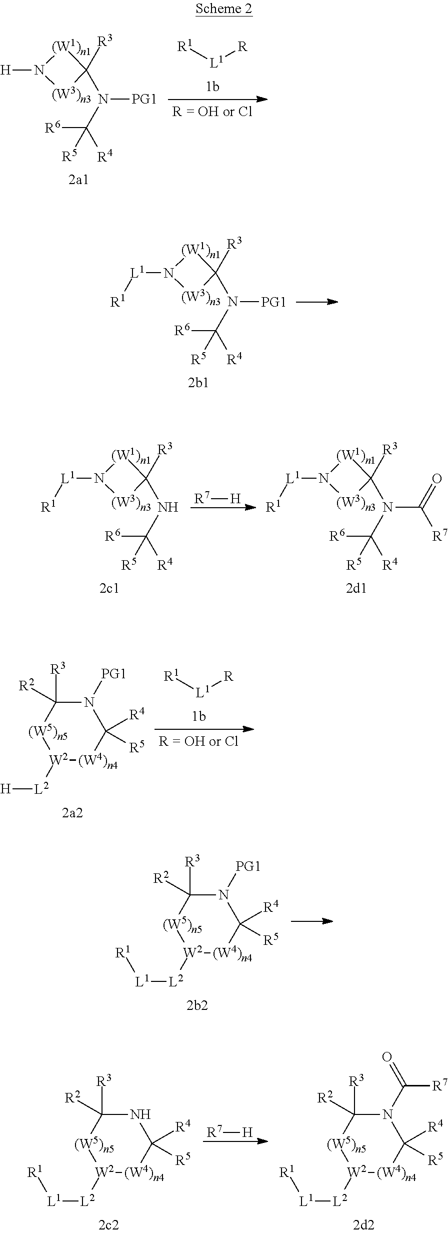

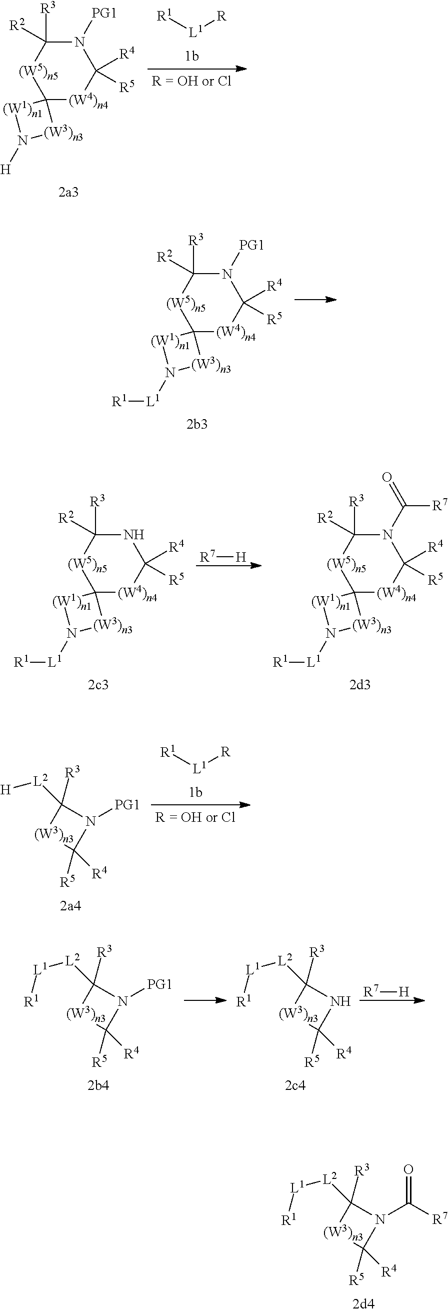

In some embodiments, a compound of Formula 2d1, 2d2, 2d3, or 2d4 may be prepared according to Scheme 2. For example, an amination reaction (e.g., using PyBOP and DBU) can be utilized to form a C—N bond between a suitably-protected amine (e.g., PG1 is Boc, Bus, Cbz, or Fmoc) of 2a1, 2a2, 2a3, or 2a4 and an aryl or heteroaryl alcohol or chloride of 1b to provide 2b1, 2b2, 2b3, or 2b4, respectively. Removal of the N-protecting group to reveal secondary amine 2c1, 2c2, 2c3, or 2c4 can be followed by reaction with R⁷—H in the presence of triphosgene (bis(trichloromethyl) carbonate (BTC) and pyridine to provide a compound of Formula 2d1, 2d2, 2d3, or 2d4.

In some embodiments, a compound of the present disclosure, for example, a compound of a formula given in Table 1, is synthesized according to one of the general routes outlined in Schemes 1 and 2, Example 1, or by methods generally known in the art. In some embodiments, exemplary compounds may include, but are not limited to, a compound selected from Table 1, or a salt or solvate thereof.

TABLE 1

| No. | Structure | Chemical Name | [M + H]⁺ |
|---|---|---|---|
| 101^{†} | 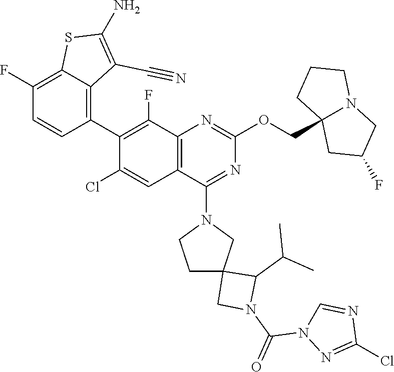 [SMILES: CC(C)C1N(C(=O)n2cnc(Cl)n2)CC12CCN(c1nc(OC[C@@]34CCCN3C[C@H](F)C4)nc3c(F)c(-c4ccc(F)c5sc(N)c(C#N)c45)c(Cl)cc13)C2] | 2-amino-4-(6-chloro-4-(2-(3-chloro-1H-1,2,4- triazole-1-carbonyl)-1-isopropyl-2,6- diazaspiro[3.4]octan-6-yl)-8-fluoro-2-(((2R,7aS)- 2-fluorotetrahydro-1H-pyrrolizin-7a(5H)- yl)methoxy)quinazolin-7-yl)-7- fluorobenzo[b]thiophene-3-carbonitrile (diastereomer 1) | 811.3 |
| 102^{†} | 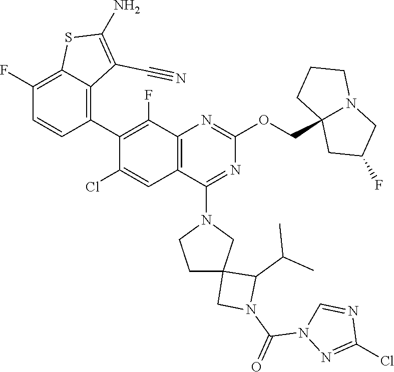 [SMILES: CC(C)C1N(C(=O)n2cnc(Cl)n2)CC12CCN(c1nc(OC[C@@]34CCCN3C[C@H](F)C4)nc3c(F)c(-c4ccc(F)c5sc(N)c(C#N)c45)c(Cl)cc13)C2] | 2-amino-4-(6-chloro-4-(2-(3-chloro-1H-1,2,4- triazole-1-carbonyl)-1-isopropyl-2,6- diazaspiro[3.4]octan-6-yl)-8-fluoro-2-(((2R,7aS)- 2-fluorotetrahydro-1H-pyrrolizin-7a(5H)- yl)methoxy)quinazolin-7-yl)-7- fluorobenzo[b]thiophene-3-carbonitrile (diastereomer 2) | 811.2 |
| 103^{†} | 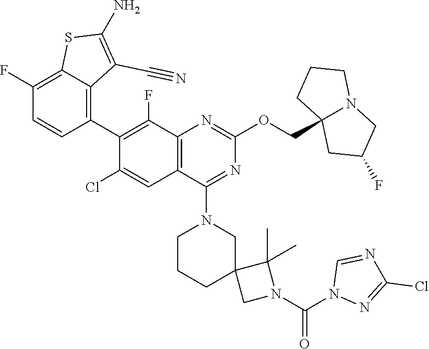 [SMILES: CC1(C)N(C(=O)n2cnc(Cl)n2)CC12CCCN(c1nc(OC[C@@]34CCCN3C[C@H](F)C4)nc3c(F)c(-c4ccc(F)c5sc(N)c(C#N)c45)c(Cl)cc13)C2] | 2-amino-4-(6-chloro-4-(2-(3-chloro-1H-1,2,4- triazole-1-carbonyl)-1,1-dimethyl-2,6- diazaspiro[3.5]nonan-6-yl)-8-fluoro-2-(((2R,7aS)- 2-fluorotetrahydro-1H-pyrrolizin-7a(5H)- yl)methoxy)quinazolin-7-yl)-7- fluorobenzo[b]thiophene-3-carbonitrile | 811.4 |
| 104^{†} | 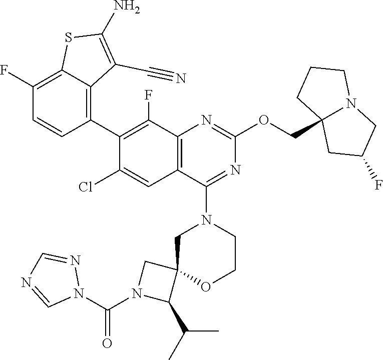 [SMILES: CC(C)[C@H]1N(C(=O)n2cncn2)C[C@@]12CN(c1nc(OC[C@@]34CCCN3C[C@H](F)C4)nc3c(F)c(-c4ccc(F)c5sc(N)c(C#N)c45)c(Cl)cc13)CCO2] | 2-amino-4-(6-chloro-8-fluoro-2-(((2R,7aS)-2- fluorotetrahydro-1H-pyrrolizin-7a(5H)- yl)methoxy)-4-((1R,4S)-1-isopropyl-2-(1H-1,2,4- triazole-1-carbonyl)-5-oxa-2,8- diazaspiro[3.5]nonan-8-yl)quinazolin-7-yl)-7- fluorobenzo[b]thiophene-3-carbonitrile | 793.8 |
| 105^{†} | 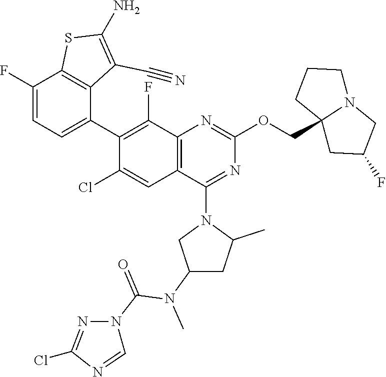 [SMILES: CC1CC(N(C)C(=O)n2cnc(Cl)n2)CN1c1nc(OC[C@@]23CCCN2C[C@H](F)C3)nc2c(F)c(-c3ccc(F)c4sc(N)c(C#N)c34)c(Cl)cc12] | N-(1-(7-(2-amino-3-cyano-7- fluorobenzo[b]thiophen-4-yl)-6-chloro-8-fluoro-2- (((2R,7aS)-2-fluorotetrahydro-1H-pyrrolizin- 7a(5H)-yl)methoxy)quinazolin-4-yl)-5- methylpyrrolidin-3-yl)-3-chloro-N-methyl-1H- 1,2,4-triazole-1-carboxamide (diastereomer 1) | 771.4 |
| 106^{†} | 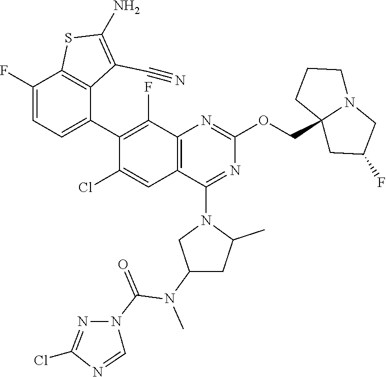 [SMILES: CC1CC(N(C)C(=O)n2cnc(Cl)n2)CN1c1nc(OC[C@@]23CCCN2C[C@H](F)C3)nc2c(F)c(-c3ccc(F)c4sc(N)c(C#N)c34)c(Cl)cc12] | N-(1-(7-(2-amino-3-cyano-7- fluorobenzo[b]thiophen-4-yl)-6-chloro-8-fluoro-2- (((2R,7aS)-2-fluorotetrahydro-1H-pyrrolizin- 7a(5H)-yl)methoxy)quinazolin-4-yl)-5- methylpyrrolidin-3-yl)-3-chloro-N-methyl-1H- 1,2,4-triazole-1-carboxamide (diastereomer 2) | 771.4 |
| 107^{†} | 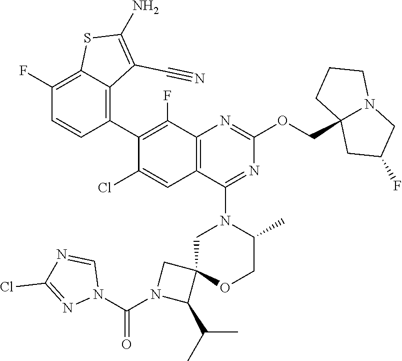 [SMILES: CC(C)[C@H]1N(C(=O)n2cnc(Cl)n2)C[C@]12CN(c1nc(OC[C@@]34CCCN3C[C@H](F)C4)nc3c(F)c(-c4ccc(F)c5sc(N)c(C#N)c45)c(Cl)cc13)[C@H](C)CO2] | 2-amino-4-(6-chloro-4-((1R,4R,7R)-2-(3-chloro- 1H-1,2,4-triazole-1-carbonyl)-1-isopropyl-7- methyl-5-oxa-2,8-diazaspiro[3.5]nonan-8-yl)-8- fluoro-2-(((2R,7aS)-2-fluorotetrahydro-1H- pyrrolizin-7a(5H)-yl)methoxy)quinazolin-7-yl)-7- fluorobenzo[b]thiophene-3-carbonitrile | 841.7 |
| 108^{†} | 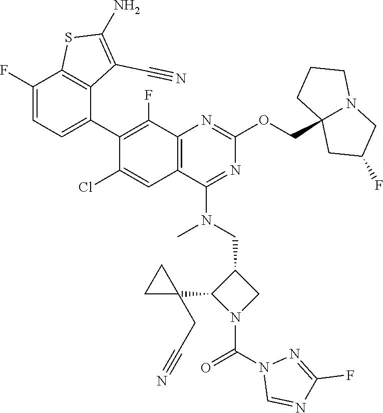 [SMILES: CN(C[C@@H]1CN(C(=O)n2cnc(F)n2)[C@@H]1C1(CC#N)CC1)c1nc(OC[C@@]23CCCN2C[C@H](F)C3)nc2c(F)c(-c3ccc(F)c4sc(N)c(C#N)c34)c(Cl)cc12] | 2-amino-4-(6-chloro-4-((((2S,3R)-2-(1- (cyanomethyl)cyclopropyl)-1-(3-fluoro-1H-1,2,4- triazole-1-carbonyl)azetidin-3- yl)methyl)(methyl)amino)-8-fluoro-2-(((2R,7aS)- 2-fluorotetrahydro-1H-pyrrolizin-7a(5H)- yl)methoxy)quinazolin-7-yl)-7- fluorobenzo[b]thiophene-3-carbonitrile | 820.3 |
| 109^{†} | 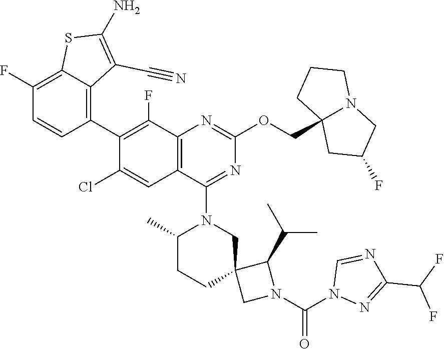 [SMILES: CC(C)[C@H]1N(C(=O)n2cnc(C(F)F)n2)C[C@]12CC[C@H](C)N(c1nc(OC[C@@]34CCCN3C[C@H](F)C4)nc3c(F)c(-c4ccc(F)c5sc(N)c(C#N)c45)c(Cl)cc13)C2] | 2-amino-4-(6-chloro-4-((1R,4R,7S)-2-(3- (difluoromethyl)-1H-1,2,4-triazole-1-carbonyl)-1- isopropyl-7-methyl-2,6-diazaspiro[3.5]nonan-6- yl)-8-fluoro-2-(((2R,7aS)-2-fluorotetrahydro-1H- pyrrolizin-7a(5H)-yl)methoxy)quinazolin-7-yl)-7- fluorobenzo[b]thiophene-3-carbonitrile | 855.4 |
| 110^{†} | 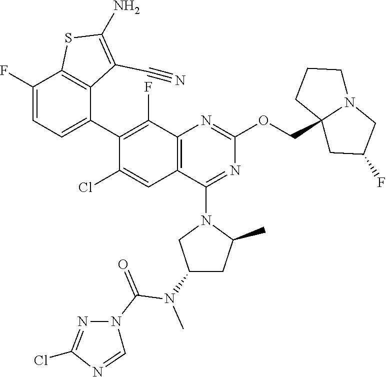 [SMILES: C[C@H]1C[C@H](N(C)C(=O)n2cnc(Cl)n2)CN1c1nc(OC[C@@]23CCCN2C[C@H](F)C3)nc2c(F)c(-c3ccc(F)c4sc(N)c(C#N)c34)c(Cl)cc12] | N-((3S,5S)-1-(7-(2-amino-3-cyano-7- fluorobenzo[b]thiophen-4-yl)-6-chloro-8-fluoro-2- (((2R,7aS)-2-fluorotetrahydro-1H-pyrrolizin- 7a(5H)-yl)methoxy)quinazolin-4-yl)-5- methylpyrrolidin-3-yl)-3-chloro-N-methyl-1H- 1,2,4-triazole-1-carboxamide | 771.3 |
| 111^{†} | 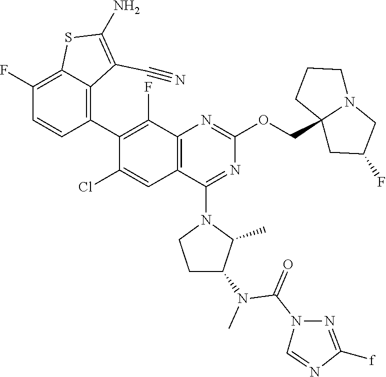 | N-((2R,3R)-1-(7-(2-amino-3-cyano-7- fluorobenzo[b]thiophen-4-yl)-6-chloro-8-fluoro-2- (((2R,7aS)-2-fluorotetrahydro-1H-pyrrolizin- 7a(5H)-yl)methoxy)quinazolin-4-yl)-2- methylpyrrolidin-3-yl)-3-fluoro-N-methyl-1H- 1,2,4-triazole-1-carboxamide | 755.8 |
| 112^{†} | 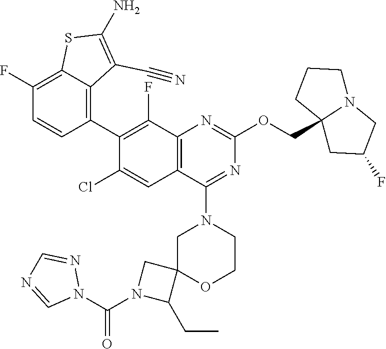 [SMILES: CCC1N(C(=O)n2cncn2)CC12CN(c1nc(OC[C@@]34CCCN3C[C@H](F)C4)nc3c(F)c(-c4ccc(F)c5sc(N)c(C#N)c45)c(Cl)cc13)CCO2] | 2-amino-4-(6-chloro-4-(1-ethyl-2-(1H-1,2,4- triazole-1-carbonyl)-5-oxa-2,8- diazaspiro[3.5]nonan-8-yl)-8-fluoro-2-(((2R,7aS)- 2-fluorotetrahydro-1H-pyrrolizin-7a(5H)- yl)methoxy)quinazolin-7-yl)-7- fluorobenzo[b]thiophene-3-carbonitrile | 779.0 |
| 113 | 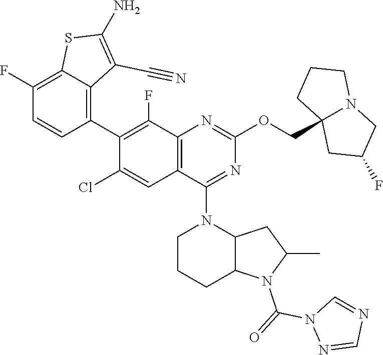 [SMILES: CC1CC2C(CCCN2c2nc(OC[C@@]34CCCN3C[C@H](F)C4)nc3c(F)c(-c4ccc(F)c5sc(N)c(C#N)c45)c(Cl)cc23)N1C(=O)n1cncn1] | 2-amino-4-(6-chloro-8-fluoro-2-(((2R,7aS)-2- fluorotetrahydro-1H-pyrrolizin-7a(5H)- yl)methoxy)-4-(2-methyl-1-(1H-1,2,4-triazole-1- carbonyl)octahydro-4H-pyrrolo[3,2-b]pyridin-4- yl)quinazolin-7-yl)-7-fluorobenzo[b]thiophene-3- carbonitrile | 763.4 |
| 114^{†} | 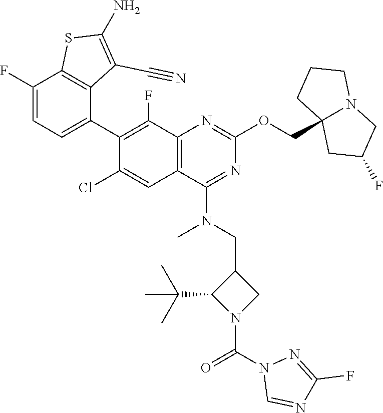 [SMILES: CN(CC1CN(C(=O)n2cnc(F)n2)[C@@H]1C(C)(C)C)c1nc(OC[C@@]23CCCN2C[C@H](F)C3)nc2c(F)c(-c3ccc(F)c4sc(N)c(C#N)c34)c(Cl)cc12] | 2-amino-4-(4-((((2S)-2-(tert-butyl)-1-(3-fluoro- 1H-1,2,4-triazole-1-carbonyl)azetidin-3- yl)methyl)(methyl)amino)-6-chloro-8-fluoro-2- (((2R,7aS)-2-fluorotetrahydro-1H-pyrrolizin- 7a(5H)-yl)methoxy)quinazolin-7-yl)-7- fluorobenzo[b]thiophene-3-carbonitrile | 797.3 |
| 115^{†} | 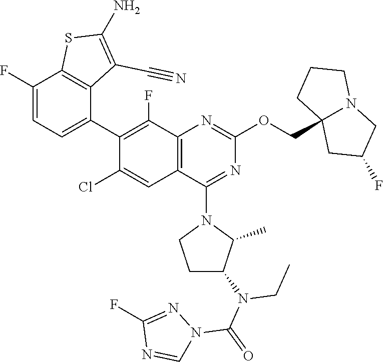 | N-((2R,3R)-1-(7-(2-amino-3-cyano-7- fluorobenzo[b]thiophen-4-yl)-6-chloro-8-fluoro-2- (((2R,7aS)-2-fluorotetrahydro-1H-pyrrolizin- 7a(5H)-yl)methoxy)quinazolin-4-yl)-2- methylpyrrolidin-3-yl)-N-ethyl-3-fluoro-1H-1,2,4- triazole-1-carboxamide | 769.3 |
| 116 | 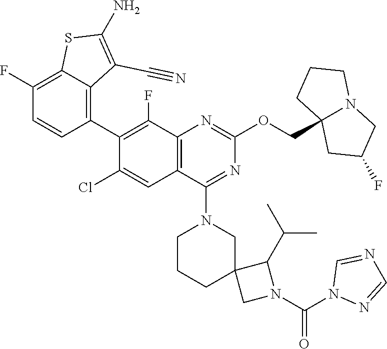 [SMILES: CC(C)C1N(C(=O)n2cncn2)CC12CCCN(c1nc(OC[C@@]34CCCN3C[C@H](F)C4)nc3c(F)c(-c4ccc(F)c5sc(N)c(C#N)c45)c(Cl)cc13)C2] | 2-amino-4-(6-chloro-8-fluoro-2-(((2R,7aS)-2- fluorotetrahydro-1H-pyrrolizin-7a(5H)- yl)methoxy)-4-(1-isopropyl-2-(1H-1,2,4-triazole- 1-carbonyl)-2,6-diazaspiro[3.5]nonan-6- yl)quinazolin-7-yl)-7-fluorobenzo[b]thiophene-3- carbonitrile | 791.4 |
| 117^{†} | 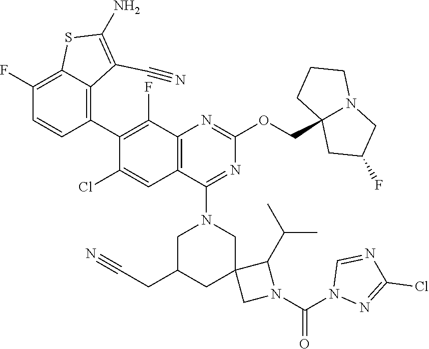 [SMILES: CC(C)C1N(C(=O)n2cnc(Cl)n2)CC12CC(CC#N)CN(c1nc(OC[C@@]34CCCN3C[C@H](F)C4)nc3c(F)c(-c4ccc(F)c5sc(N)c(C#N)c45)c(Cl)cc13)C2] | 2-amino-4-(6-chloro-4-(2-(3-chloro-1H-1,2,4- triazole-1-carbonyl)-8-(cyanomethyl)-1-isopropyl- 2,6-diazaspiro[3.5]nonan-6-yl)-8-fluoro-2- (((2R,7aS)-2-fluorotetrahydro-1H-pyrrolizin- 7a(5H)-yl)methoxy)quinazolin-7-yl)-7- fluorobenzo[b]thiophene-3-carbonitrile (diastereomer 1) | 864.4 |
| 118^{†} | 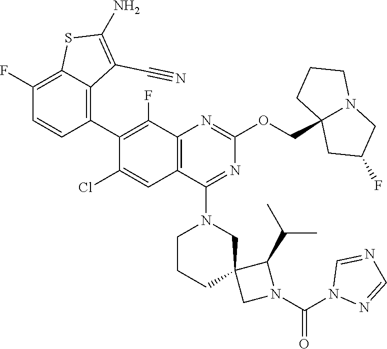 [SMILES: CC(C)[C@H]1N(C(=O)n2cncn2)C[C@]12CCCN(c1nc(OC[C@@]34CCCN3C[C@H](F)C4)nc3c(F)c(-c4ccc(F)c5sc(N)c(C#N)c45)c(Cl)cc13)C2] | 2-amino-4-(6-chloro-8-fluoro-2-(((2R,7aS)-2- fluorotetrahydro-1H-pyrrolizin-7a(5H)- yl)methoxy)-4-((1R,4R)-1-isopropyl-2-(1H-1,2,4- triazole-1-carbonyl)-2,6-diazaspiro[3.5]nonan-6- yl)quinazolin-7-yl)-7-fluorobenzo[b]thiophene-3- carbonitrile | 791.8 |
| 119 | 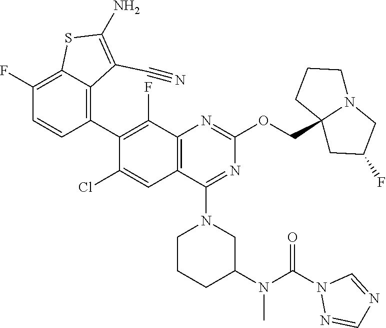 [SMILES: CN(C(=O)n1cncn1)C1CCCN(c2nc(OC[C@@]34CCCN3C[C@H](F)C4)nc3c(F)c(-c4ccc(F)c5sc(N)c(C#N)c45)c(Cl)cc23)C1] | N-(1-(7-(2-amino-3-cyano-7- fluorobenzo[b]thiophen-4-yl)-6-chloro-8-fluoro-2- (((2R,7aS)-2-fluorotetrahydro-1H-pyrrolizin- 7a(5H)-yl)methoxy)quinazolin-4-yl)piperidin-3- yl)-N-methyl-1H-1,2,4-triazole-1-carboxamide | 737.2 |
| 120^{†} | 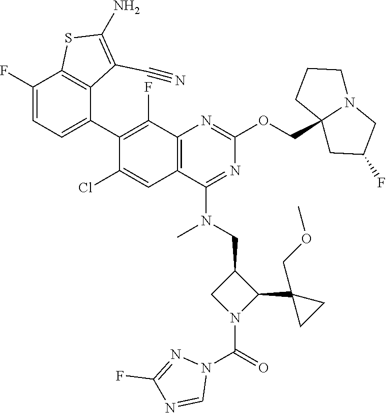 [SMILES: COCC1([C@@H]2[C@H](CN(C)c3nc(OC[C@@]45CCCN4C[C@H](F)C5)nc4c(F)c(-c5ccc(F)c6sc(N)c(C#N)c56)c(Cl)cc34)CN2C(=O)n2cnc(F)n2)CC1] | 2-amino-4-(6-chloro-8-fluoro-4-((((2S,3R)-1-(3- fluoro-1H-1,2,4-triazole-1-carbonyl)-2-(1- (methoxymethyl)cyclopropyl)azetidin-3- yl)methyl)(methyl)amino)-2-(((2R,7aS)-2- fluorotetrahydro-1H-pyrrolizin-7a(5H)- yl)methoxy)quinazolin-7-yl)-7- fluorobenzo[b]thiophene-3-carbonitrile | 825.3 |
| 121^{†} | 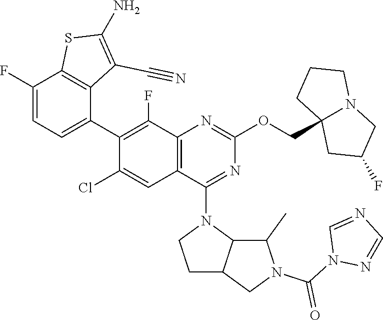 [SMILES: CC1C2C(CCN2c2nc(OC[C@@]34CCCN3C[C@H](F)C4)nc3c(F)c(-c4ccc(F)c5sc(N)c(C#N)c45)c(Cl)cc23)CN1C(=O)n1cncn1] | 2-amino-4-(6-chloro-8-fluoro-2-(((2R,7aS)-2- fluorotetrahydro-1H-pyrrolizin-7a(5H)- yl)methoxy)-4-(6-methyl-5-(1H-1,2,4-triazole-1- carbonyl)hexahydropyrrolo[3,4-b]pyrrol-1(2H)- yl)quinazolin-7-yl)-7-fluorobenzo[b]thiophene-3- carbonitrile (diastereomer 2) | 749.4 |
| 122^{†} | 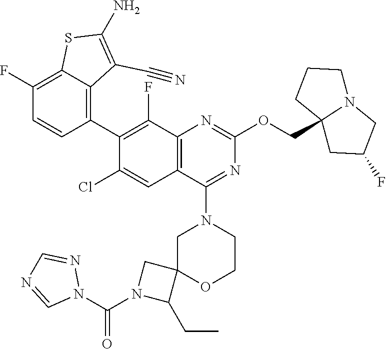 [SMILES: CCC1N(C(=O)n2cncn2)CC12CN(c1nc(OC[C@@]34CCCN3C[C@H](F)C4)nc3c(F)c(-c4ccc(F)c5sc(N)c(C#N)c45)c(Cl)cc13)CCO2] | 2-amino-4-(6-chloro-4-(1-ethyl-2-(1H-1,2,4- triazole-1-carbonyl)-5-oxa-2,8- diazaspiro[3.5]nonan-8-yl)-8-fluoro-2-(((2R,7aS)- 2-fluorotetrahydro-1H-pyrrolizin-7a(5H)- yl)methoxy)quinazolin-7-yl)-7- fluorobenzo[b]thiophene-3-carbonitrile | 779.6 |
| 123^{†} | 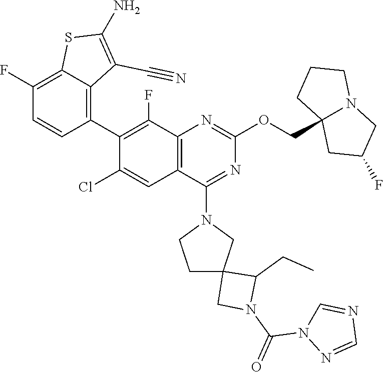 [SMILES: CCC1N(C(=O)n2cncn2)CC12CCN(c1nc(OC[C@@]34CCCN3C[C@H](F)C4)nc3c(F)c(-c4ccc(F)c5sc(N)c(C#N)c45)c(Cl)cc13)C2] | 2-amino-4-(6-chloro-4-(1-ethyl-2-(1H-1,2,4- triazole-1-carbonyl)-2,6-diazaspiro[3.4]octan-6- yl)-8-fluoro-2-(((2R,7aS)-2-fluorotetrahydro-1H- pyrrolizin-7a(5H)-yl)methoxy)quinazolin-7-yl)-7- fluorobenzo[b]thiophene-3-carbonitrile | 763.2 |
| 124 | 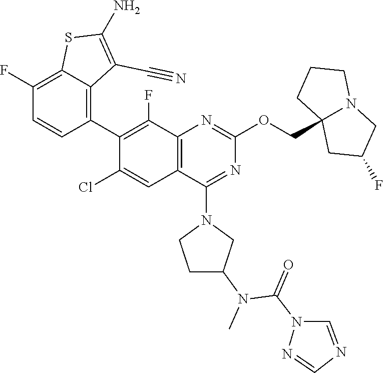 [SMILES: CN(C(=O)n1cncn1)C1CCN(c2nc(OC[C@@]34CCCN3C[C@H](F)C4)nc3c(F)c(-c4ccc(F)c5sc(N)c(C#N)c45)c(Cl)cc23)C1] | N-(1-(7-(2-amino-3-cyano-7- fluorobenzo[b]thiophen-4-yl)-6-chloro-8-fluoro-2- (((2R,7aS)-2-fluorotetrahydro-1H-pyrrolizin- 7a(5H)-yl)methoxy)quinazolin-4-yl)pyrrolidin-3- yl)-N-methyl-1H-1,2,4-triazole-1-carboxamide | 723.4 |
| 125^{†} | 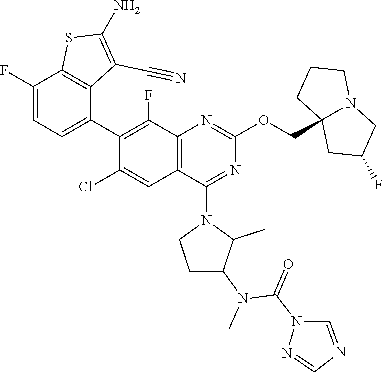 [SMILES: CC1C(N(C)C(=O)n2cncn2)CCN1c1nc(OC[C@@]23CCCN2C[C@H](F)C3)nc2c(F)c(-c3ccc(F)c4sc(N)c(C#N)c34)c(Cl)cc12] | N-(1-(7-(2-amino-3-cyano-7- fluorobenzo[b]thiophen-4-yl)-6-chloro-8-fluoro-2- (((2R,7aS)-2-fluorotetrahydro-1H-pyrrolizin- 7a(5H)-yl)methoxy)quinazolin-4-yl)-2- methylpyrrolidin-3-yl)-N-methyl-1H-1,2,4- triazole-1-carboxamide | 737.4 |
| 126^{†} | 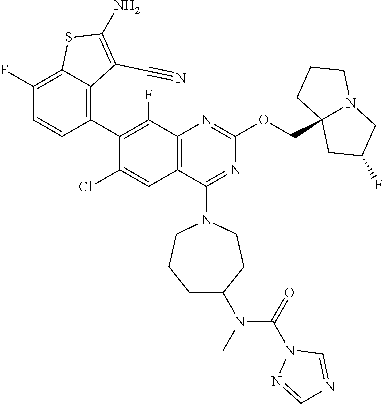 [SMILES: CN(C(=O)n1cncn1)C1CCCN(c2nc(OC[C@@]34CCCN3C[C@H](F)C4)nc3c(F)c(-c4ccc(F)c5sc(N)c(C#N)c45)c(Cl)cc23)CC1] | N-(1-(7-(2-amino-3-cyano-7- fluorobenzo[b]thiophen-4-yl)-6-chloro-8-fluoro-2- (((2R,7aS)-2-fluorotetrahydro-1H-pyrrolizin- 7a(5H)-yl)methoxy)quinazolin-4-yl)azepan-4-yl)- N-methyl-1H-1,2,4-triazole-1-carboxamide | 751.5 |
| 127^{†} | 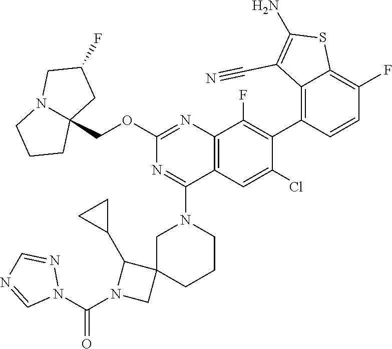 [SMILES: N#Cc1c(N)sc2c(F)ccc(-c3c(Cl)cc4c(N5CCCC6(C5)CN(C(=O)n5cncn5)C6C5CC5)nc(OC[C@@]56CCCN5C[C@H](F)C6)nc4c3F)c12] | 2-amino-4-(6-chloro-4-(1-cyclopropyl-2-(1H- 1,2,4-triazole-1-carbonyl)-2,6- diazaspiro[3.5]nonan-6-yl)-8-fluoro-2-(((2R,7aS)- 2-fluorotetrahydro-1H-pyrrolizin-7a(5H)- yl)methoxy)quinazolin-7-yl)-7- fluorobenzo[b]thiophene-3-carbonitrile | 789.4 |
| 128 | 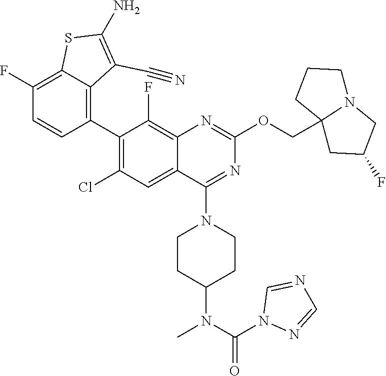 [SMILES: CN(C(=O)n1cncn1)C1CCN(c2nc(OCC34CCCN3C[C@H](F)C4)nc3c(F)c(-c4ccc(F)c5sc(N)c(C#N)c45)c(Cl)cc23)CC1] | N-(1-(7-(2-amino-3-cyano-7- fluorobenzo[b]thiophen-4-yl)-6-chloro-8-fluoro-2- (((2R)-2-fluorotetrahydro-1H-pyrrolizin-7a(5H)- yl)methoxy)quinazolin-4-yl)piperidin-4-yl)-N- methyl-1H-1,2,4-triazole-1-carboxamide | 737.4 |
| 129^{†} | 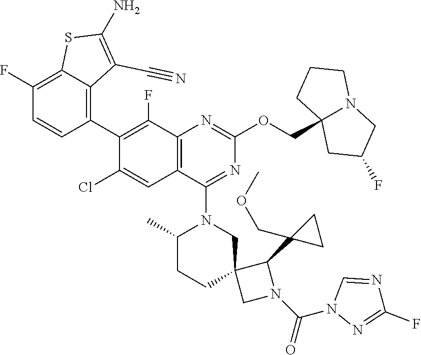 [SMILES: COCC1([C@H]2N(C(=O)n3cnc(F)n3)C[C@]23CC[C@H](C)N(c2nc(OC[C@@]45CCCN4C[C@H](F)C5)nc4c(F)c(-c5ccc(F)c6sc(N)c(C#N)c56)c(Cl)cc24)C3)CC1] | 2-amino-4-(6-chloro-8-fluoro-4-((1S,4R,7S)-2-(3- fluoro-1H-1,2,4-triazole-1-carbonyl)-1-(1- (methoxymethyl)cyclopropyl)-7-methyl-2,6- diazaspiro[3.5]nonan-6-yl)-2-(((2R,7aS)-2- fluorotetrahydro-1H-pyrrolizin-7a(5H)- yl)methoxy)quinazolin-7-yl)-7- fluorobenzo[b]thiophene-3-carbonitrile | 865.2 |
| 130^{†} | 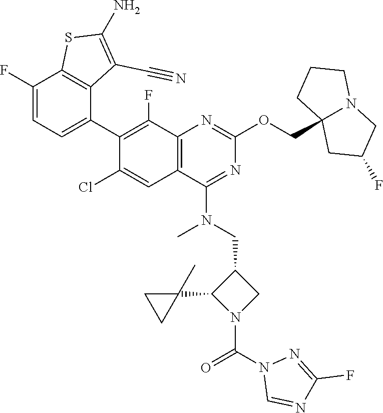 [SMILES: CN(C[C@@H]1CN(C(=O)n2cnc(F)n2)[C@@H]1C1(C)CC1)c1nc(OC[C@@]23CCCN2C[C@H](F)C3)nc2c(F)c(-c3ccc(F)c4sc(N)c(C#N)c34)c(Cl)cc12] | 2-amino-4-(6-chloro-8-fluoro-4-((((2S,3R)-1-(3- fluoro-1H-1,2,4-triazole-1-carbonyl)-2-(1- methylcyclopropyl)azetidin-3- yl)methyl)(methyl)amino)-2-(((2R,7aS)-2- fluorotetrahydro-1H-pyrrolizin-7a(5H)- yl)methoxy)quinazolin-7-yl)-7- fluorobenzo[b]thiophene-3-carbonitrile | 795.2 |
| 131^{†} | 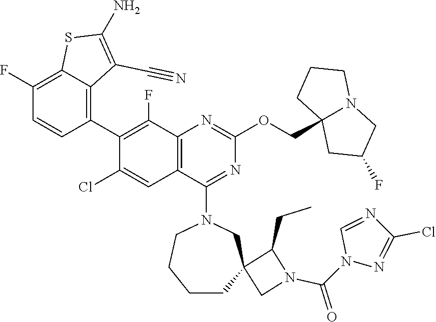 [SMILES: CC[C@H]1N(C(=O)n2cnc(Cl)n2)C[C@]12CCCCN(c1nc(OC[C@@]34CCCN3C[C@H](F)C4)nc3c(F)c(-c4ccc(F)c5sc(N)c(C#N)c45)c(Cl)cc13)C2] | 2-amino-4-(6-chloro-4-((1R,4R)-2-(3-chloro-1H- 1,2,4-triazole-1-carbonyl)-1-ethyl-2,6- diazaspiro[3.6]decan-6-yl)-8-fluoro-2-(((2R,7aS)- 2-fluorotetrahydro-1H-pyrrolizin-7a(5H)- yl)methoxy)quinazolin-7-yl)-7- fluorobenzo[b]thiophene-3-carbonitrile | 825.4 |
| 132^{†} | 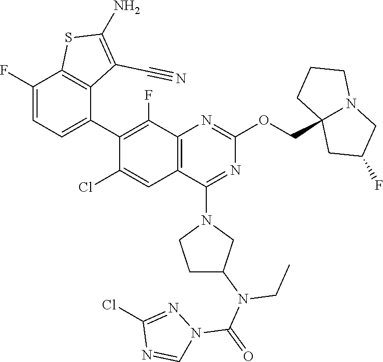 | N-(1-(7-(2-amino-3-cyano-7- fluorobenzo[b]thiophen-4-yl)-6-chloro-8-fluoro-2- (((2R,7aS)-2-fluorotetrahydro-1H-pyrrolizin- 7a(5H)-yl)methoxy)quinazolin-4-yl)pyrrolidin-3- yl)-3-chloro-N-ethyl-1H-1,2,4-triazole-1- carboxamide | 771.3 |
| 133^{†} | 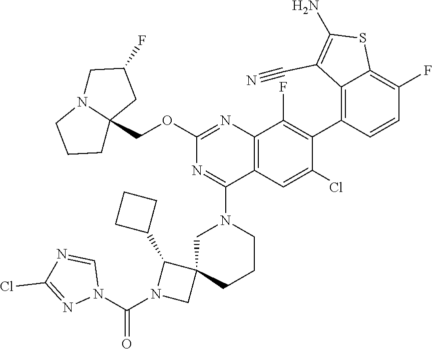 [SMILES: N#Cc1c(N)sc2c(F)ccc(-c3c(Cl)cc4c(N5CCC[C@@]6(C5)CN(C(=O)n5cnc(Cl)n5)[C@@H]6C5CCC5)nc(OC[C@@]56CCCN5C[C@H](F)C6)nc4c3F)c12] | 2-amino-4-(6-chloro-4-((1R,4R)-2-(3-chloro-1H- 1,2,4-triazole-1-carbonyl)-1-cyclobutyl-2,6- diazaspiro[3.5]nonan-6-yl)-8-fluoro-2-(((2R,7aS)- 2-fluorotetrahydro-1H-pyrrolizin-7a(5H)- yl)methoxy)quinazolin-7-yl)-7- fluorobenzo[b]thiophene-3-carbonitrile | 837.2 |
| 134^{†} | 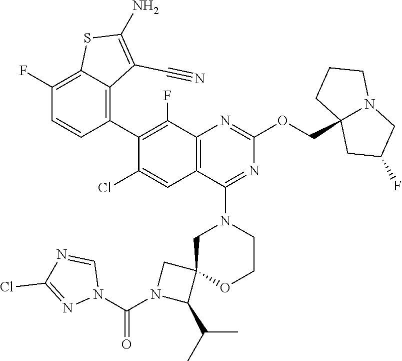 [SMILES: CC(C)[C@H]1N(C(=O)n2cnc(Cl)n2)C[C@@]12CN(c1nc(OC[C@@]34CCCN3C[C@H](F)C4)nc3c(F)c(-c4ccc(F)c5sc(N)c(C#N)c45)c(Cl)cc13)CCO2] | 2-amino-4-(6-chloro-4-((1R,4S)-2-(3-chloro-1H- 1,2,4-triazole-1-carbonyl)-1-isopropyl-5-oxa-2,8- diazaspiro[3.5]nonan-8-yl)-8-fluoro-2-(((2R,7aS)- 2-fluorotetrahydro-1H-pyrrolizin-7a(5H)- yl)methoxy)quinazolin-7-yl)-7- fluorobenzo[b]thiophene-3-carbonitrile | 827.4 |
| 135^{†} | 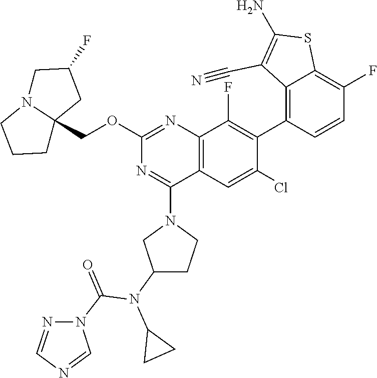 [SMILES: N#Cc1c(N)sc2c(F)ccc(-c3c(Cl)cc4c(N5CCC(N(C(=O)n6cncn6)C6CC6)C5)nc(OC[C@@]56CCCN5C[C@H](F)C6)nc4c3F)c12] | N-(1-(7-(2-amino-3-cyano-7- fluorobenzo[b]thiophen-4-yl)-6-chloro-8-fluoro-2- (((2R,7aS)-2-fluorotetrahydro-1H-pyrrolizin- 7a(5H)-yl)methoxy)quinazolin-4-yl)pyrrolidin-3- yl)-N-cyclopropyl-1H-1,2,4-triazole-1- carboxamide | 749.9 |
| 136^{†} | 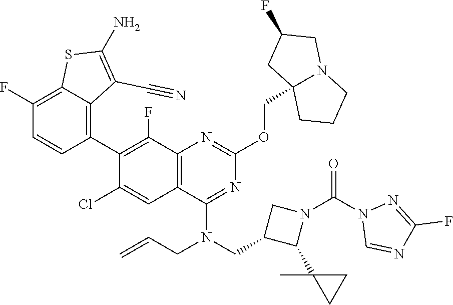 [SMILES: C=CCN(C[C@@H]1CN(C(=O)n2cnc(F)n2)[C@@H]1C1(C)CC1)c1nc(OC[C@@]23CCCN2C[C@H](F)C3)nc2c(F)c(-c3ccc(F)c4sc(N)c(C#N)c34)c(Cl)cc12] | 4-(4-(allyl(((2S,3R)-1-(3-fluoro-1H-1,2,4-triazole- 1-carbonyl)-2-(1-methylcyclopropyl)azetidin-3- yl)methyl)amino)-6-chloro-8-fluoro-2-(((2R,7aS)- 2-fluorotetrahydro-1H-pyrrolizin-7a(5H)- yl)methoxy)quinazolin-7-yl)-2-amino-7- fluorobenzo[b]thiophene-3-carbonitrile | 821.3 |
| 137^{†} | 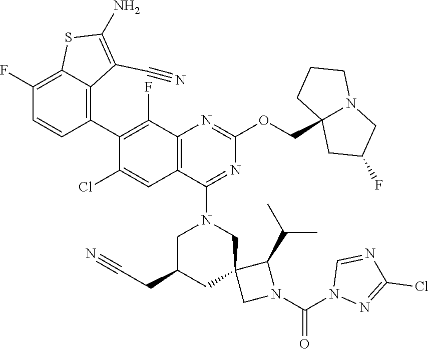 [SMILES: CC(C)[C@H]1N(C(=O)n2cnc(Cl)n2)C[C@]12C[C@@H](CC#N)CN(c1nc(OC[C@@]34CCCN3C[C@H](F)C4)nc3c(F)c(-c4ccc(F)c5sc(N)c(C#N)c45)c(Cl)cc13)C2] | 2-amino-4-(6-chloro-4-((1R,4R,8S)-2-(3-chloro- 1H-1,2,4-triazole-1-carbonyl)-8-(cyanomethyl)-1- isopropyl-2,6-diazaspiro[3.5]nonan-6-yl)-8-fluoro- 2-(((2R,7aS)-2-fluorotetrahydro-1H-pyrrolizin- 7a(5H)-yl)methoxy)quinazolin-7-yl)-7- fluorobenzo[b]thiophene-3-carbonitrile | 864.4 |
| 138^{†} | 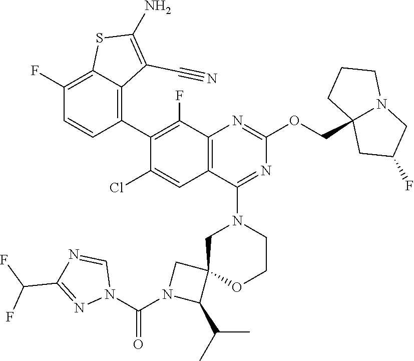 [SMILES: CC(C)[C@H]1N(C(=O)n2cnc(C(F)F)n2)C[C@@]12CN(c1nc(OC[C@@]34CCCN3C[C@H](F)C4)nc3c(F)c(-c4ccc(F)c5sc(N)c(C#N)c45)c(Cl)cc13)CCO2] | 2-amino-4-(6-chloro-4-((1R,4S)-2-(3- (difluoromethyl)-1H-1,2,4-triazole-1-carbonyl)-1- isopropyl-5-oxa-2,8-diazaspiro[3.5]nonan-8-yl)-8- fluoro-2-(((2R,7aS)-2-fluorotetrahydro-1H- pyrrolizin-7a(5H)-yl)methoxy)quinazolin-7-yl)-7- fluorobenzo[b]thiophene-3-carbonitrile | 843.4 |
| 139^{†} | 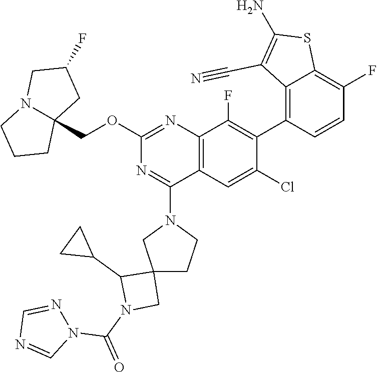 [SMILES: N#Cc1c(N)sc2c(F)ccc(-c3c(Cl)cc4c(N5CCC6(C5)CN(C(=O)n5cncn5)C6C5CC5)nc(OC[C@@]56CCCN5C[C@H](F)C6)nc4c3F)c12] | 2-amino-4-(6-chloro-4-(1-cyclopropyl-2-(1H- 1,2,4-triazole-1-carbonyl)-2,6- diazaspiro[3.4]octan-6-yl)-8-fluoro-2-(((2R,7aS)- 2-fluorotetrahydro-1H-pyrrolizin-7a(5H)- yl)methoxy)quinazolin-7-yl)-7- fluorobenzo[b]thiophene-3-carbonitrile (diastereomer 1) | 775.8 |
| 140^{†} | 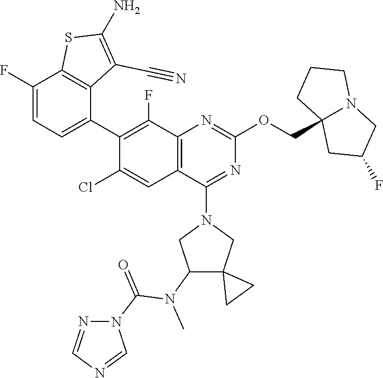 [SMILES: CN(C(=O)n1cncn1)C1CN(c2nc(OC[C@@]34CCCN3C[C@H](F)C4)nc3c(F)c(-c4ccc(F)c5sc(N)c(C#N)c45)c(Cl)cc23)CC12CC2] | N-(5-(7-(2-amino-3-cyano-7- fluorobenzo[b]thiophen-4-yl)-6-chloro-8-fluoro-2- (((2R,7aS)-2-fluorotetrahydro-1H-pyrrolizin- 7a(5H)-yl)methoxy)quinazolin-4-yl)-5- azaspiro[2.4]heptan-7-yl)-N-methyl-1H-1,2,4- triazole-1-carboxamide | 749.4 |
| 141^{†} | 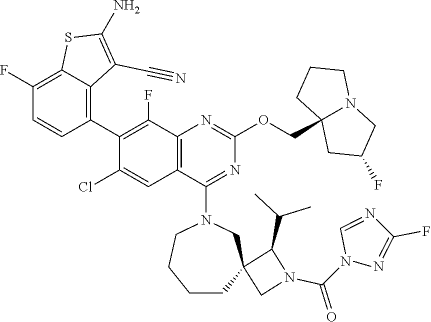 [SMILES: CC(C)[C@H]1N(C(=O)n2cnc(F)n2)C[C@]12CCCCN(c1nc(OC[C@@]34CCCN3C[C@H](F)C4)nc3c(F)c(-c4ccc(F)c5sc(N)c(C#N)c45)c(Cl)cc13)C2] | 2-amino-4-(6-chloro-8-fluoro-4-((1R,4R)-2-(3- fluoro-1H-1,2,4-triazole-1-carbonyl)-1-isopropyl- 2,6-diazaspiro[3.6]decan-6-yl)-2-(((2R,7aS)-2- fluorotetrahydro-1H-pyrrolizin-7a(5H)- yl)methoxy)quinazolin-7-yl)-7- fluorobenzo[b]thiophene-3-carbonitrile | 823.2 |
| 142^{†} | 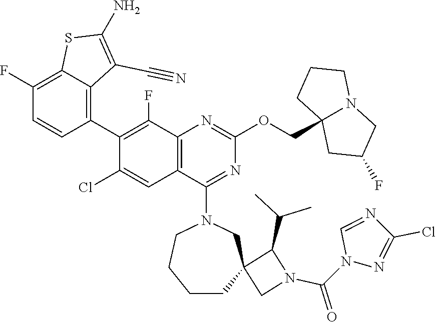 [SMILES: CC(C)[C@H]1N(C(=O)n2cnc(Cl)n2)C[C@]12CCCCN(c1nc(OC[C@@]34CCCN3C[C@H](F)C4)nc3c(F)c(-c4ccc(F)c5sc(N)c(C#N)c45)c(Cl)cc13)C2] | 2-amino-4-(6-chloro-4-((1R,4R)-2-(3-chloro-1H- 1,2,4-triazole-1-carbonyl)-1-isopropyl-2,6- diazaspiro[3.6]decan-6-yl)-8-fluoro-2-(((2R,7aS)- 2-fluorotetrahydro-1H-pyrrolizin-7a(5H)- yl)methoxy)quinazolin-7-yl)-7- fluorobenzo[b]thiophene-3-carbonitrile | 839.2 |
| 143^{†} | 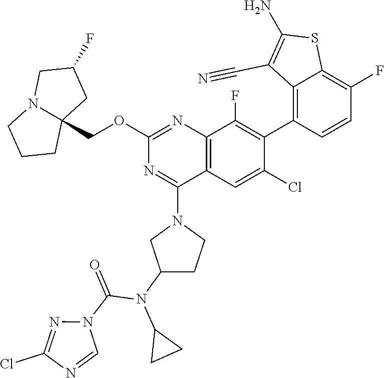 [SMILES: N#Cc1c(N)sc2c(F)ccc(-c3c(Cl)cc4c(N5CCC(N(C(=O)n6cnc(Cl)n6)C6CC6)C5)nc(OC[C@@]56CCCN5C[C@H](F)C6)nc4c3F)c12] | N-(1-(7-(2-amino-3-cyano-7- fluorobenzo[b]thiophen-4-yl)-6-chloro-8-fluoro-2- (((2R,7aS)-2-fluorotetrahydro-1H-pyrrolizin- 7a(5H)-yl)methoxy)quinazolin-4-yl)pyrrolidin-3- yl)-3-chloro-N-cyclopropyl-1H-1,2,4-triazole-1- carboxamide | 783.2 |
| 144^{†} | 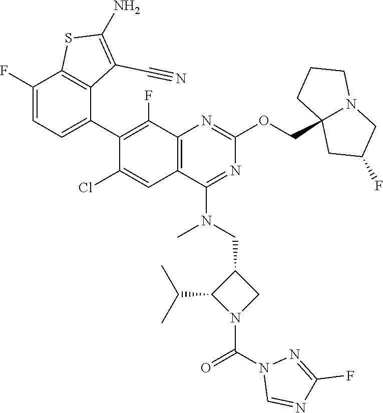 [SMILES: CC(C)[C@@H]1[C@H](CN(C)c2nc(OC[C@@]34CCCN3C[C@H](F)C4)nc3c(F)c(-c4ccc(F)c5sc(N)c(C#N)c45)c(Cl)cc23)CN1C(=O)n1cnc(F)n1] | 2-amino-4-(6-chloro-8-fluoro-4-((((2R,3R)-1-(3- fluoro-1H-1,2,4-triazole-1-carbonyl)-2- isopropylazetidin-3-yl)methyl)(methyl)amino)-2- (((2R,7aS)-2-fluorotetrahydro-1H-pyrrolizin- 7a(5H)-yl)methoxy)quinazolin-7-yl)-7- fluorobenzo[b]thiophene-3-carbonitrile | 783.3 |
| 145 | 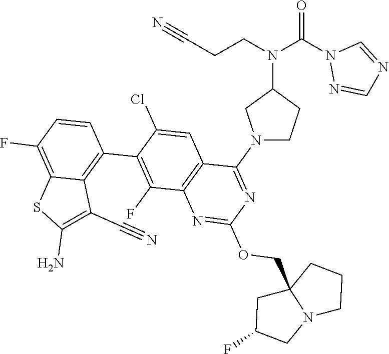 [SMILES: N#CCCN(C(=O)n1cncn1)C1CCN(c2nc(OC[C@@]34CCCN3C[C@H](F)C4)nc3c(F)c(-c4ccc(F)c5sc(N)c(C#N)c45)c(Cl)cc23)C1] | N-(1-(7-(2-amino-3-cyano-7- fluorobenzo[b]thiophen-4-yl)-6-chloro-8-fluoro-2- (((2R,7aS)-2-fluorotetrahydro-1H-pyrrolizin- 7a(5H)-yl)methoxy)quinazolin-4-yl)pyrrolidin-3- yl)-N-(2-cyanoethyl)-1H-1,2,4-triazole-1- carboxamide | 762.2 |
| 146^{†} | 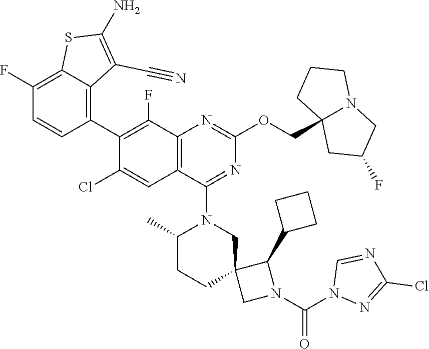 [SMILES: C[C@H]1CC[C@]2(CN(C(=O)n3cnc(Cl)n3)[C@@H]2C2CCC2)CN1c1nc(OC[C@@]23CCCN2C[C@H](F)C3)nc2c(F)c(-c3ccc(F)c4sc(N)c(C#N)c34)c(Cl)cc12] | 2-amino-4-(6-chloro-4-((1R,4R,7S)-2-(3-chloro- 1H-1,2,4-triazole-1-carbonyl)-1-cyclobutyl-7- methyl-2,6-diazaspiro[3.5]nonan-6-yl)-8-fluoro-2- (((2R,7aS)-2-fluorotetrahydro-1H-pyrrolizin- 7a(5H)-yl)methoxy)quinazolin-7-yl)-7- fluorobenzo[b]thiophene-3-carbonitrile | 851.6 |
| 147^{†} | 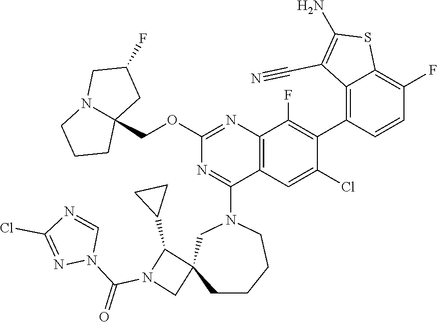 [SMILES: N#Cc1c(N)sc2c(F)ccc(-c3c(Cl)cc4c(N5CCCC[C@@]6(C5)CN(C(=O)n5cnc(Cl)n5)[C@@H]6C5CC5)nc(OC[C@@]56CCCN5C[C@H](F)C6)nc4c3F)c12] | 2-amino-4-(6-chloro-4-((1R,4R)-2-(3-chloro-1H- 1,2,4-triazole-1-carbonyl)-1-cyclopropyl-2,6- diazaspiro[3.6]decan-6-yl)-8-fluoro-2-(((2R,7aS)- 2-fluorotetrahydro-1H-pyrrolizin-7a(5H)- yl)methoxy)quinazolin-7-yl)-7- fluorobenzo[b]thiophene-3-carbonitrile | 837.2 |
| 148^{†} | 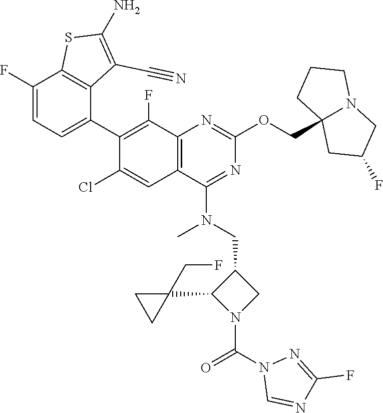 [SMILES: CN(C[C@@H]1CN(C(=O)n2cnc(F)n2)[C@@H]1C1(CF)CC1)c1nc(OC[C@@]23CCCN2C[C@H](F)C3)nc2c(F)c(-c3ccc(F)c4sc(N)c(C#N)c34)c(Cl)cc12] | 2-amino-4-(6-chloro-8-fluoro-4-((((2S,3R)-1-(3- fluoro-1H-1,2,4-triazole-1-carbonyl)-2-(1- (fluoromethyl)cyclopropyl)azetidin-3- yl)methyl)(methyl)amino)-2-(((2R,7aS)-2- fluorotetrahydro-1H-pyrrolizin-7a(5H)- yl)methoxy)quinazolin-7-yl)-7- fluorobenzo[b]thiophene-3-carbonitrile | 813.2 |
| 149^{†} | 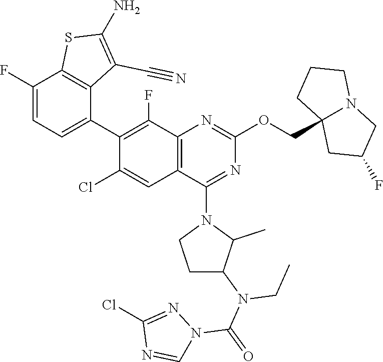 | N-(1-(7-(2-amino-3-cyano-7- fluorobenzo[b]thiophen-4-yl)-6-chloro-8-fluoro-2- (((2R,7aS)-2-fluorotetrahydro-1H-pyrrolizin- 7a(5H)-yl)methoxy)quinazolin-4-yl)-2- methylpyrrolidin-3-yl)-3-chloro-N-ethyl-1H-1,2,4- triazole-1-carboxamide | 785.1 |
| 150^{†} | 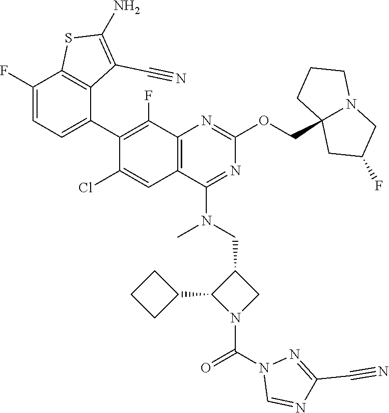 [SMILES: CN(C[C@@H]1CN(C(=O)n2cnc(C#N)n2)[C@@H]1C1CCC1)c1nc(OC[C@@]23CCCN2C[C@H](F)C3)nc2c(F)c(-c3ccc(F)c4sc(N)c(C#N)c34)c(Cl)cc12] | 1-((2R,3R)-3-(((7-(2-amino-3-cyano-7- fluorobenzo[b]thiophen-4-yl)-6-chloro-8-fluoro-2- (((2R,7aS)-2-fluorotetrahydro-1H-pyrrolizin- 7a(5H)-yl)methoxy)quinazolin-4- yl)(methyl)amino)methyl)-2-cyclobutylazetidine- 1-carbonyl)-1H-1,2,4-triazole-3-carbonitrile | 802.3 |
| 151^{†} | 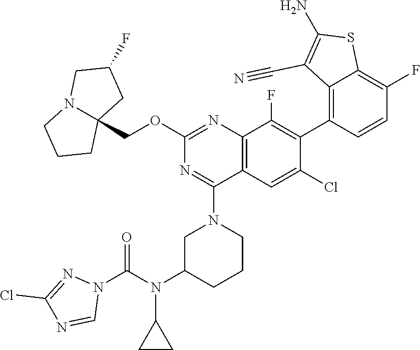 [SMILES: N#Cc1c(N)sc2c(F)ccc(-c3c(Cl)cc4c(N5CCCC(N(C(=O)n6cnc(Cl)n6)C6CC6)C5)nc(OC[C@@]56CCCN5C[C@H](F)C6)nc4c3F)c12] | N-(1-(7-(2-amino-3-cyano-7- fluorobenzo[b]thiophen-4-yl)-6-chloro-8-fluoro-2- (((2R,7aS)-2-fluorotetrahydro-1H-pyrrolizin- 7a(5H)-yl)methoxy)quinazolin-4-yl)piperidin-3- yl)-3-chloro-N-cyclopropyl-1H-1,2,4-triazole-1- carboxamide | 797.4 |
| 152^{†} | 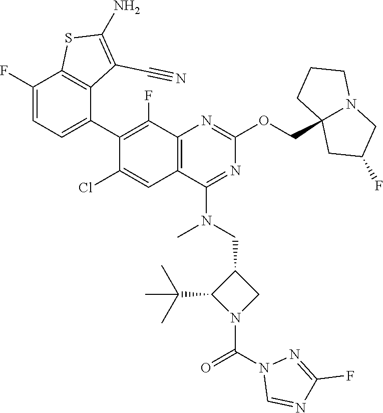 [SMILES: CN(C[C@@H]1CN(C(=O)n2cnc(F)n2)[C@@H]1C(C)(C)C)c1nc(OC[C@@]23CCCN2C[C@H](F)C3)nc2c(F)c(-c3ccc(F)c4sc(N)c(C#N)c34)c(Cl)cc12] | 2-amino-4-(4-((((2S,3R)-2-(tert-butyl)-1-(3-fluoro- 1H-1,2,4-triazole-1-carbonyl)azetidin-3- yl)methyl)(methyl)amino)-6-chloro-8-fluoro-2- (((2R,7aS)-2-fluorotetrahydro-1H-pyrrolizin- 7a(5H)-yl)methoxy)quinazolin-7-yl)-7- fluorobenzo[b]thiophene-3-carbonitrile | 797.2 |
| 153^{†} | 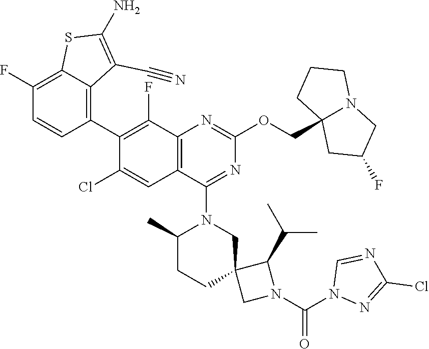 [SMILES: CC(C)[C@H]1N(C(=O)n2cnc(Cl)n2)C[C@]12CC[C@@H](C)N(c1nc(OC[C@@]34CCCN3C[C@H](F)C4)nc3c(F)c(-c4ccc(F)c5sc(N)c(C#N)c45)c(Cl)cc13)C2] | 2-amino-4-(6-chloro-4-((1R,4R,7R)-2-(3-chloro- 1H-1,2,4-triazole-1-carbonyl)-1-isopropyl-7- methyl-2,6-diazaspiro[3.5]nonan-6-yl)-8-fluoro-2- (((2R,7aS)-2-fluorotetrahydro-1H-pyrrolizin- 7a(5H)-yl)methoxy)quinazolin-7-yl)-7- fluorobenzo[b]thiophene-3-carbonitrile | 839.8 |
| 154^{†} | 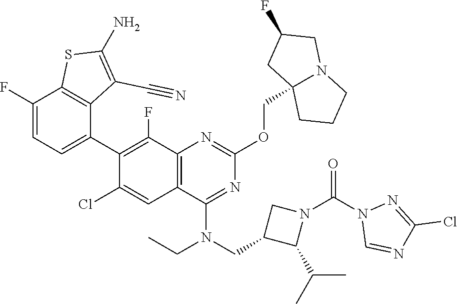 [SMILES: CCN(C[C@@H]1CN(C(=O)n2cnc(Cl)n2)[C@@H]1C(C)C)c1nc(OC[C@@]23CCCN2C[C@H](F)C3)nc2c(F)c(-c3ccc(F)c4sc(N)c(C#N)c34)c(Cl)cc12] | 2-amino-4-(6-chloro-4-((((2R,3R)-1-(3-chloro-1H- 1,2,4-triazole-1-carbonyl)-2-isopropylazetidin-3- yl)methyl)(ethyl)amino)-8-fluoro-2-(((2R,7aS)-2- fluorotetrahydro-1H-pyrrolizin-7a(5H)- yl)methoxy)quinazolin-7-yl)-7- fluorobenzo[b]thiophene-3-carbonitrile | 813.4 |
| 155^{†} | 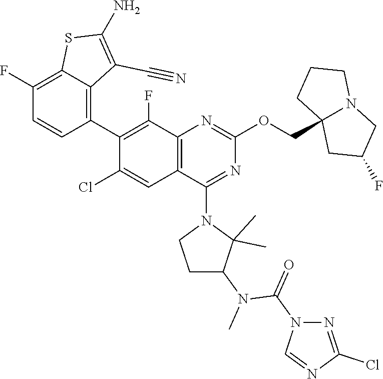 [SMILES: CN(C(=O)n1cnc(Cl)n1)C1CCN(c2nc(OC[C@@]34CCCN3C[C@H](F)C4)nc3c(F)c(-c4ccc(F)c5sc(N)c(C#N)c45)c(Cl)cc23)C1(C)C] | N-(1-(7-(2-amino-3-cyano-7- fluorobenzo[b]thiophen-4-yl)-6-chloro-8-fluoro-2- (((2R,7aS)-2-fluorotetrahydro-1H-pyrrolizin- 7a(5H)-yl)methoxy)quinazolin-4-yl)-2,2- dimethylpyrrolidin-3-yl)-3-chloro-N-methyl-1H- 1,2,4-triazole-1-carboxamide (diastereomer 1) | 785.6 |
| 156^{†} | 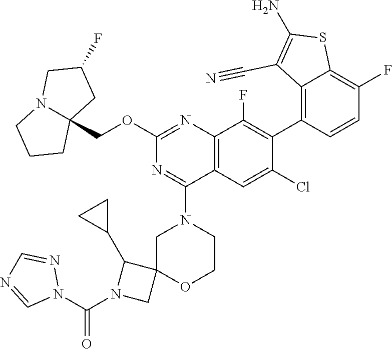 [SMILES: N#Cc1c(N)sc2c(F)ccc(-c3c(Cl)cc4c(N5CCOC6(C5)CN(C(=O)n5cncn5)C6C5CC5)nc(OC[C@@]56CCCN5C[C@H](F)C6)nc4c3F)c12] | 2-amino-4-(6-chloro-4-(1-cyclopropyl-2-(1H- 1,2,4-triazole-1-carbonyl)-5-oxa-2,8- diazaspiro[3.5]nonan-8-yl)-8-fluoro-2-(((2R,7aS)- 2-fluorotetrahydro-1H-pyrrolizin-7a(5H)- yl)methoxy)quinazolin-7-yl)-7- fluorobenzo[b]thiophene-3-carbonitrile | 791.2 |
| 157^{†} | 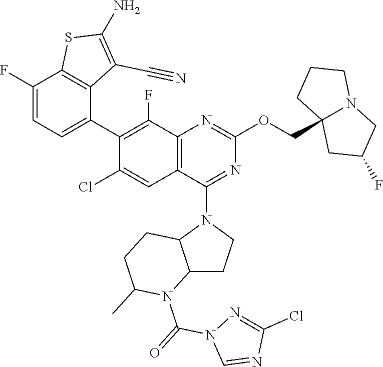 [SMILES: CC1CCC2C(CCN2c2nc(OC[C@@]34CCCN3C[C@H](F)C4)nc3c(F)c(-c4ccc(F)c5sc(N)c(C#N)c45)c(Cl)cc23)N1C(=O)n1cnc(Cl)n1] | 2-amino-4-(6-chloro-4-(4-(3-chloro-1H-1,2,4- triazole-1-carbonyl)-5-methyloctahydro-1H- pyrrolo[3,2-b]pyridin-1-yl)-8-fluoro-2-(((2R,7aS)- 2-fluorotetrahydro-1H-pyrrolizin-7a(5H)- yl)methoxy)quinazolin-7-yl)-7- fluorobenzo[b]thiophene-3-carbonitrile | 797.3 |
| 158^{†} | 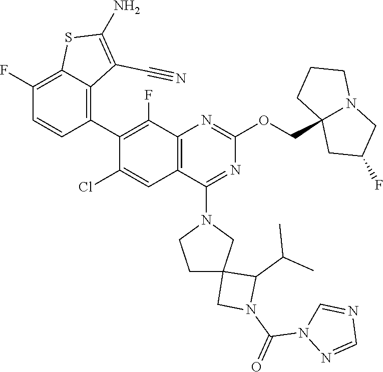 [SMILES: CC(C)C1N(C(=O)n2cncn2)CC12CCN(c1nc(OC[C@@]34CCCN3C[C@H](F)C4)nc3c(F)c(-c4ccc(F)c5sc(N)c(C#N)c45)c(Cl)cc13)C2] | 2-amino-4-(6-chloro-8-fluoro-2-(((2R,7aS)-2- fluorotetrahydro-1H-pyrrolizin-7a(5H)- yl)methoxy)-4-(1-isopropyl-2-(1H-1,2,4-triazole- 1-carbonyl)-2,6-diazaspiro[3.4]octan-6- yl)quinazolin-7-yl)-7-fluorobenzo[b]thiophene-3- carbonitrile | 777.4 |
| 159^{†} | 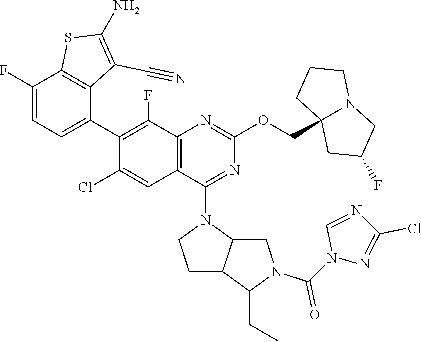 [SMILES: CCC1C2CCN(c3nc(OC[C@@]45CCCN4C[C@H](F)C5)nc4c(F)c(-c5ccc(F)c6sc(N)c(C#N)c56)c(Cl)cc34)C2CN1C(=O)n1cnc(Cl)n1] | 2-amino-4-(6-chloro-4-(5-(3-chloro-1H-1,2,4- triazole-1-carbonyl)-4-ethylhexahydropyrrolo[3,4- b]pyrrol-1(2H)-yl)-8-fluoro-2-(((2R,7aS)-2- fluorotetrahydro-1H-pyrrolizin-7a(5H)- yl)methoxy)quinazolin-7-yl)-7- fluorobenzo[b]thiophene-3-carbonitrile | 797.3 |
| 160^{†} | 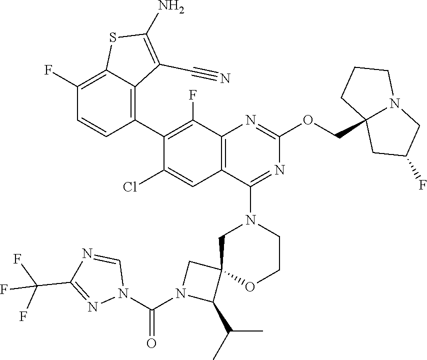 [SMILES: CC(C)[C@H]1N(C(=O)n2cnc(C(F)(F)F)n2)C[C@@]12CN(c1nc(OC[C@@]34CCCN3C[C@H](F)C4)nc3c(F)c(-c4ccc(F)c5sc(N)c(C#N)c45)c(Cl)cc13)CCO2] | 2-amino-4-(6-chloro-8-fluoro-2-(((2R,7aS)-2- fluorotetrahydro-1H-pyrrolizin-7a(5H)- yl)methoxy)-4-((1R,4S)-1-isopropyl-2-(3- (trifluoromethyl)-1H-1,2,4-triazole-1-carbonyl)-5- oxa-2,8-diazaspiro[3.5]nonan-8-yl)quinazolin-7- yl)-7-fluorobenzo[b]thiophene-3-carbonitrile | 862.7 |
| 161 | 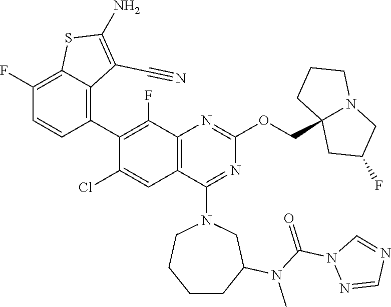 [SMILES: CN(C(=O)n1cncn1)C1CCCCN(c2nc(OC[C@@]34CCCN3C[C@H](F)C4)nc3c(F)c(-c4ccc(F)c5sc(N)c(C#N)c45)c(Cl)cc23)C1] | N-(1-(7-(2-amino-3-cyano-7- fluorobenzo[b]thiophen-4-yl)-6-chloro-8-fluoro-2- (((2R,7aS)-2-fluorotetrahydro-1H-pyrrolizin- 7a(5H)-yl)methoxy)quinazolin-4-yl)azepan-3-yl)- N-methyl-1H-1,2,4-triazole-1-carboxamide | 751.6 |
| 162^{†} | 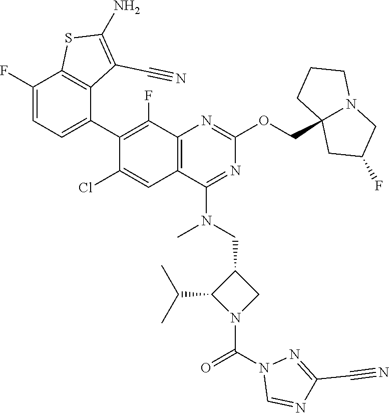 [SMILES: CC(C)[C@@H]1[C@H](CN(C)c2nc(OC[C@@]34CCCN3C[C@H](F)C4)nc3c(F)c(-c4ccc(F)c5sc(N)c(C#N)c45)c(Cl)cc23)CN1C(=O)n1cnc(C#N)n1] | 1-((2R,3R)-3-(((7-(2-amino-3-cyano-7- fluorobenzo[b]thiophen-4-yl)-6-chloro-8-fluoro-2- (((2R,7aS)-2-fluorotetrahydro-1H-pyrrolizin- 7a(5H)-yl)methoxy)quinazolin-4- yl)(methyl)amino)methyl)-2-isopropylazetidine-1- carbonyl)-1H-1,2,4-triazole-3-carbonitrile | 790.2 |
| 163^{†} | 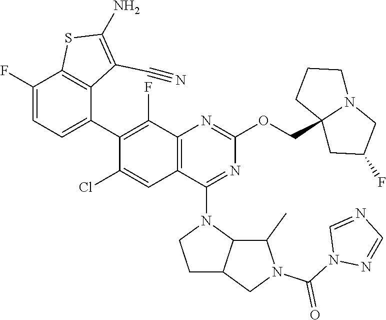 [SMILES: CC1C2C(CCN2c2nc(OC[C@@]34CCCN3C[C@H](F)C4)nc3c(F)c(-c4ccc(F)c5sc(N)c(C#N)c45)c(Cl)cc23)CN1C(=O)n1cncn1] | 2-amino-4-(6-chloro-8-fluoro-2-(((2R,7aS)-2- fluorotetrahydro-1H-pyrrolizin-7a(5H)- yl)methoxy)-4-(6-methyl-5-(1H-1,2,4-triazole-1- carbonyl)hexahydropyrrolo[3,4-b]pyrrol-1(2H)- yl)quinazolin-7-yl)-7-fluorobenzo[b]thiophene-3- carbonitrile (diastereomer 1) | 749.3 |
| 164^{†} | 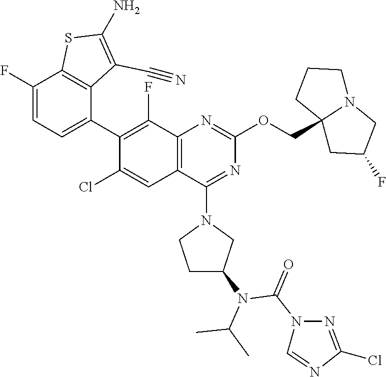 [SMILES: CC(C)N(C(=O)n1cnc(Cl)n1)[C@H]1CCN(c2nc(OC[C@@]34CCCN3C[C@H](F)C4)nc3c(F)c(-c4ccc(F)c5sc(N)c(C#N)c45)c(Cl)cc23)C1] | N-((3S)-1-(7-(2-amino-3-cyano-7- fluorobenzo[b]thiophen-4-yl)-6-chloro-8-fluoro-2- (((2R,7aS)-2-fluorotetrahydro-1H-pyrrolizin- 7a(5H)-yl)methoxy)quinazolin-4-yl)pyrrolidin-3- yl)-3-chloro-N-isopropyl-1H-1,2,4-triazole-1- carboxamide | 785.2 |
| 165^{†} | 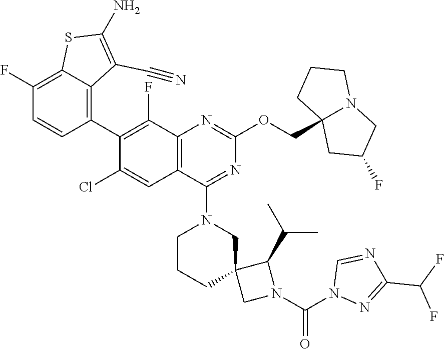 [SMILES: CC(C)[C@H]1N(C(=O)n2cnc(C(F)F)n2)C[C@]12CCCN(c1nc(OC[C@@]34CCCN3C[C@H](F)C4)nc3c(F)c(-c4ccc(F)c5sc(N)c(C#N)c45)c(Cl)cc13)C2] | 2-amino-4-(6-chloro-4-((1R,4R)-2-(3- (difluoromethyl)-1H-1,2,4-triazole-1-carbonyl)-1- isopropyl-2,6-diazaspiro[3.5]nonan-6-yl)-8-fluoro- 2-(((2R,7aS)-2-fluorotetrahydro-1H-pyrrolizin- 7a(5H)-yl)methoxy)quinazolin-7-yl)-7- fluorobenzo[b]thiophene-3-carbonitrile | 841.4 |
| 166 | 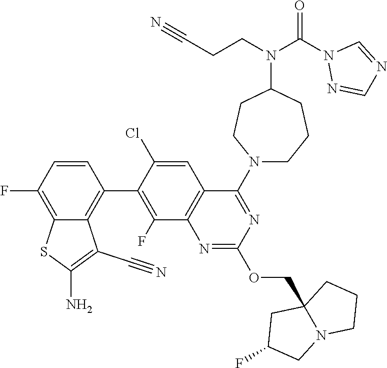 [SMILES: N#CCCN(C(=O)n1cncn1)C1CCCN(c2nc(OC[C@@]34CCCN3C[C@H](F)C4)nc3c(F)c(-c4ccc(F)c5sc(N)c(C#N)c45)c(Cl)cc23)CC1] | N-(1-(7-(2-amino-3-cyano-7- fluorobenzo[b]thiophen-4-yl)-6-chloro-8-fluoro-2- (((2R,7aS)-2-fluorotetrahydro-1H-pyrrolizin- 7a(5H)-yl)methoxy)quinazolin-4-yl)azepan-4-yl)- N-(2-cyanoethyl)-1H-1,2,4-triazole-1-carboxamide | 790.2 |
| 167^{†} | 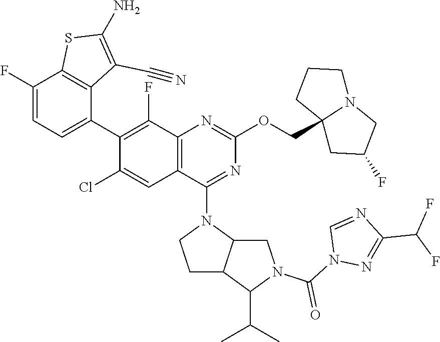 [SMILES: CC(C)C1C2CCN(c3nc(OC[C@@]45CCCN4C[C@H](F)C5)nc4c(F)c(-c5ccc(F)c6sc(N)c(C#N)c56)c(Cl)cc34)C2CN1C(=O)n1cnc(C(F)F)n1] | 2-amino-4-(6-chloro-4-(5-(3-(difluoromethyl)-1H- 1,2,4-triazole-1-carbonyl)-4- isopropylhexahydropyrrolo[3,4-b]pyrrol-1(2H)- yl)-8-fluoro-2-(((2R,7aS)-2-fluorotetrahydro-1H- pyrrolizin-7a(5H)-yl)methoxy)quinazolin-7-yl)-7- fluorobenzo[b]thiophene-3-carbonitrile (diastereomer 1) | 827.2 |
| 168^{†} | 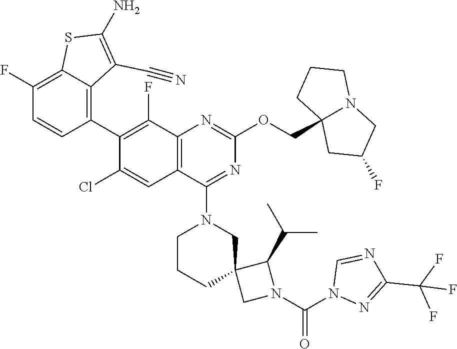 [SMILES: CC(C)[C@H]1N(C(=O)n2cnc(C(F)(F)F)n2)C[C@]12CCCN(c1nc(OC[C@@]34CCCN3C[C@H](F)C4)nc3c(F)c(-c4ccc(F)c5sc(N)c(C#N)c45)c(Cl)cc13)C2] | 2-amino-4-(6-chloro-8-fluoro-2-(((2R,7aS)-2- fluorotetrahydro-1H-pyrrolizin-7a(5H)- yl)methoxy)-4-((1R,4R)-1-isopropyl-2-(3- (trifluoromethyl)-1H-1,2,4-triazole-1-carbonyl)- 2,6-diazaspiro[3.5]nonan-6-yl)quinazolin-7-yl)-7- fluorobenzo[b]thiophene-3-carbonitrile | 859.4 |
| 169^{†} | 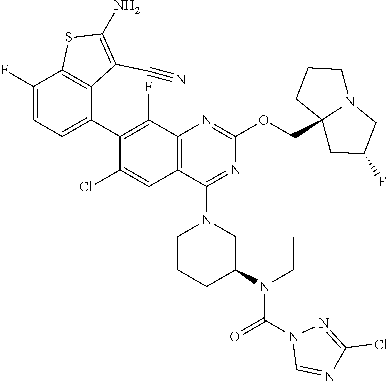 [SMILES: CCN(C(=O)n1cnc(Cl)n1)[C@H]1CCCN(c2nc(OC[C@@]34CCCN3C[C@H](F)C4)nc3c(F)c(-c4ccc(F)c5sc(N)c(C#N)c45)c(Cl)cc23)C1] | N-((3S)-1-(7-(2-amino-3-cyano-7- fluorobenzo[b]thiophen-4-yl)-6-chloro-8-fluoro-2- (((2R,7aS)-2-fluorotetrahydro-1H-pyrrolizin- 7a(5H)-yl)methoxy)quinazolin-4-yl)piperidin-3- yl)-3-chloro-N-ethyl-1H-1,2,4-triazole-1- carboxamide | 785.3 |
| 170^{†} | 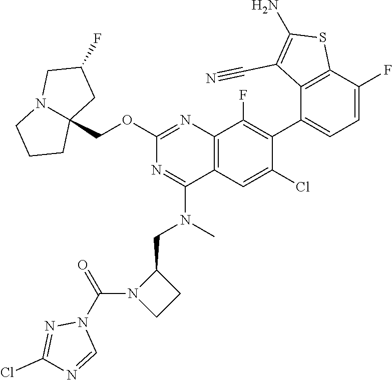 [SMILES: CN(C[C@H]1CCN1C(=O)n1cnc(Cl)n1)c1nc(OC[C@@]23CCCN2C[C@H](F)C3)nc2c(F)c(-c3ccc(F)c4sc(N)c(C#N)c34)c(Cl)cc12] | 2-amino-4-(6-chloro-4-((((R)-1-(3-chloro-1H- 1,2,4-triazole-1-carbonyl)azetidin-2- yl)methyl)(methyl)amino)-8-fluoro-2-(((2R,7aS)- 2-fluorotetrahydro-1H-pyrrolizin-7a(5H)- yl)methoxy)quinazolin-7-yl)-7- fluorobenzo[b]thiophene-3-carbonitrile | 757.3 |
| 171^{†} | 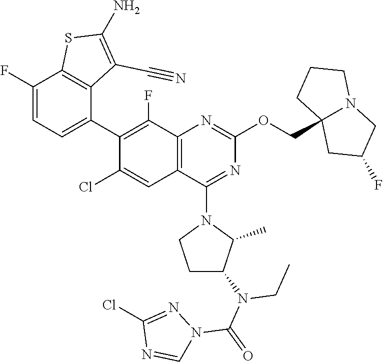 | N-((2R,3R)-1-(7-(2-amino-3-cyano-7- fluorobenzo[b]thiophen-4-yl)-6-chloro-8-fluoro-2- (((2R,7aS)-2-fluorotetrahydro-1H-pyrrolizin- 7a(5H)-yl)methoxy)quinazolin-4-yl)-2- methylpyrrolidin-3-yl)-3-chloro-N-ethyl-1H-1,2,4- triazole-1-carboxamide (diastereomer 2) | 785.3 |
| 172^{†} | 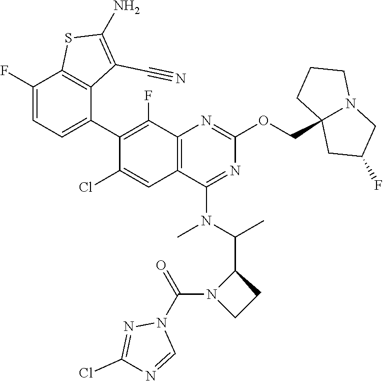 [SMILES: CC([C@H]1CCN1C(=O)n1cnc(Cl)n1)N(C)c1nc(OC[C@@]23CCCN2C[C@H](F)C3)nc2c(F)c(-c3ccc(F)c4sc(N)c(C#N)c34)c(Cl)cc12] | 2-amino-4-(6-chloro-4-((1-((R)-1-(3-chloro-1H- 1,2,4-triazole-1-carbonyl)azetidin-2- yl)ethyl)(methyl)amino)-8-fluoro-2-(((2R,7aS)-2- fluorotetrahydro-1H-pyrrolizin-7a(5H)- yl)methoxy)quinazolin-7-yl)-7- fluorobenzo[b]thiophene-3-carbonitrile | 771.3 |
| 173^{†} | 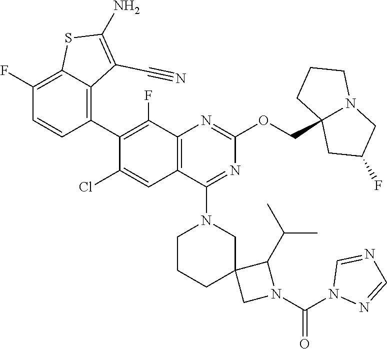 [SMILES: CC(C)C1N(C(=O)n2cncn2)CC12CCCN(c1nc(OC[C@@]34CCCN3C[C@H](F)C4)nc3c(F)c(-c4ccc(F)c5sc(N)c(C#N)c45)c(Cl)cc13)C2] | 2-amino-4-(6-chloro-8-fluoro-2-(((2R,7aS)-2- fluorotetrahydro-1H-pyrrolizin-7a(5H)- yl)methoxy)-4-(1-isopropyl-2-(1H-1,2,4-triazole- 1-carbonyl)-2,6-diazaspiro[3.5]nonan-6- yl)quinazolin-7-yl)-7-fluorobenzo[b]thiophene-3- carbonitrile | 791.4 |
| 174^{†} | 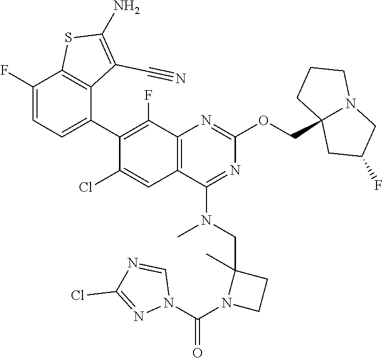 [SMILES: CN(CC1(C)CCN1C(=O)n1cnc(Cl)n1)c1nc(OC[C@@]23CCCN2C[C@H](F)C3)nc2c(F)c(-c3ccc(F)c4sc(N)c(C#N)c34)c(Cl)cc12] | 2-amino-4-(6-chloro-4-(((1-(3-chloro-1H-1,2,4- triazole-1-carbonyl)-2-methylazetidin-2- yl)methyl)(methyl)amino)-8-fluoro-2-(((2R,7aS)- 2-fluorotetrahydro-1H-pyrrolizin-7a(5H)- yl)methoxy)quinazolin-7-yl)-7- fluorobenzo[b]thiophene-3-carbonitrile (diastereomer 1) | 771.2 |
| 175^{†} | 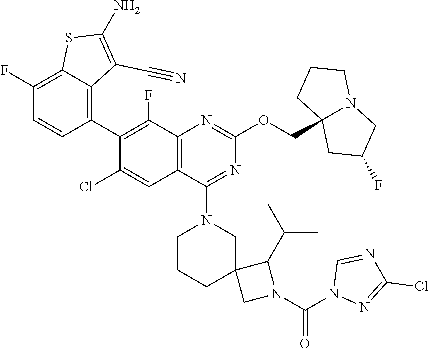 [SMILES: CC(C)C1N(C(=O)n2cnc(Cl)n2)CC12CCCN(c1nc(OC[C@@]34CCCN3C[C@H](F)C4)nc3c(F)c(-c4ccc(F)c5sc(N)c(C#N)c45)c(Cl)cc13)C2] | 2-amino-4-(6-chloro-4-(2-(3-chloro-1H-1,2,4- triazole-1-carbonyl)-1-isopropyl-2,6- diazaspiro[3.5]nonan-6-yl)-8-fluoro-2-(((2R,7aS)- 2-fluorotetrahydro-1H-pyrrolizin-7a(5H)- yl)methoxy)quinazolin-7-yl)-7- fluorobenzo[b]thiophene-3-carbonitrile | 825.3 |
| 176^{†} | 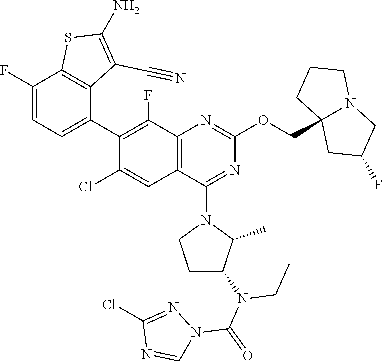 | N-((2R,3R)-1-(7-(2-amino-3-cyano-7- fluorobenzo[b]thiophen-4-yl)-6-chloro-8-fluoro-2- (((2R,7aS)-2-fluorotetrahydro-1H-pyrrolizin- 7a(5H)-yl)methoxy)quinazolin-4-yl)-2- methylpyrrolidin-3-yl)-3-chloro-N-ethyl-1H-1,2,4- triazole-1-carboxamide | 785.6 |
| 177^{†} | 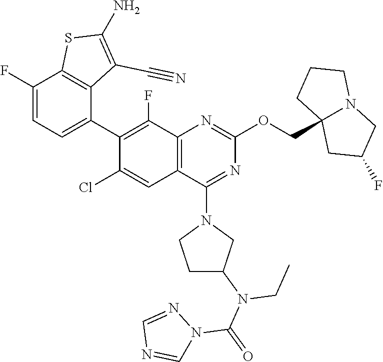 | N-(1-(7-(2-amino-3-cyano-7- fluorobenzo[b]thiophen-4-yl)-6-chloro-8-fluoro-2- (((2R,7aS)-2-fluorotetrahydro-1H-pyrrolizin- 7a(5H)-yl)methoxy)quinazolin-4-yl)pyrrolidin-3- yl)-N-ethyl-1H-1,2,4-triazole-1-carboxamide | 737.2 |
| 178^{†} | 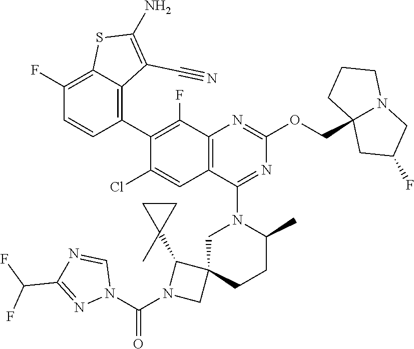 [SMILES: C[C@H]1CC[C@]2(CN(C(=O)n3cnc(C(F)F)n3)[C@@H]2C2(C)CC2)CN1c1nc(OC[C@@]23CCCN2C[C@H](F)C3)nc2c(F)c(-c3ccc(F)c4sc(N)c(C#N)c34)c(Cl)cc12] | 2-amino-4-(6-chloro-4-((1R,4R,7S)-2-(3- (difluoromethyl)-1H-1,2,4-triazole-1-carbonyl)-7- methyl-1-(1-methylcyclopropyl)-2,6- diazaspiro[3.5]nonan-6-yl)-8-fluoro-2-(((2R,7aS)- 2-fluorotetrahydro-1H-pyrrolizin-7a(5H)- yl)methoxy)quinazolin-7-yl)-7- fluorobenzo[b]thiophene-3-carbonitrile | 867.4 |
| 179^{†} | 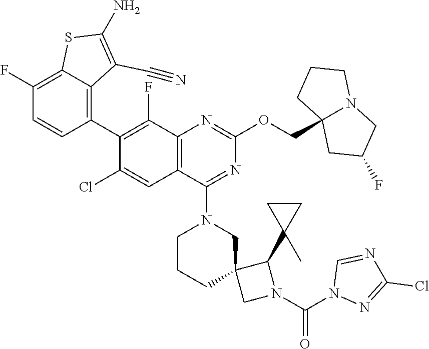 [SMILES: CC1([C@H]2N(C(=O)n3cnc(Cl)n3)C[C@]23CCCN(c2nc(OC[C@@]45CCCN4C[C@H](F)C5)nc4c(F)c(-c5ccc(F)c6sc(N)c(C#N)c56)c(Cl)cc24)C3)CC1] | 2-amino-4-(6-chloro-4-((1R,4R)-2-(3-chloro-1H- 1,2,4-triazole-1-carbonyl)-1-(1- methylcyclopropyl)-2,6-diazaspiro[3.5]nonan-6- yl)-8-fluoro-2-(((2R,7aS)-2-fluorotetrahydro-1H- pyrrolizin-7a(5H)-yl)methoxy)quinazolin-7-yl)-7- fluorobenzo[b]thiophene-3-carbonitrile | 837.2 |
| 180^{†} | 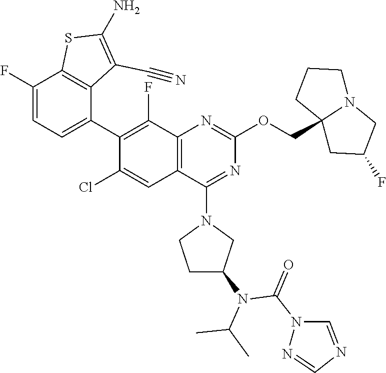 [SMILES: CC(C)N(C(=O)n1cncn1)[C@H]1CCN(c2nc(OC[C@@]34CCCN3C[C@H](F)C4)nc3c(F)c(-c4ccc(F)c5sc(N)c(C#N)c45)c(Cl)cc23)C1] | N-((3S)-1-(7-(2-amino-3-cyano-7- fluorobenzo[b]thiophen-4-yl)-6-chloro-8-fluoro-2- (((2R,7aS)-2-fluorotetrahydro-1H-pyrrolizin- 7a(5H)-yl)methoxy)quinazolin-4-yl)pyrrolidin-3- yl)-N-isopropyl-1H-1,2,4-triazole-1-carboxamide | 751.4 |
| 181^{†} | 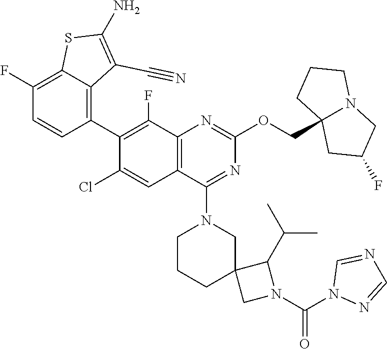 [SMILES: CC(C)C1N(C(=O)n2cncn2)CC12CCCN(c1nc(OC[C@@]34CCCN3C[C@H](F)C4)nc3c(F)c(-c4ccc(F)c5sc(N)c(C#N)c45)c(Cl)cc13)C2] | 2-amino-4-(6-chloro-8-fluoro-2-(((2R,7aS)-2- fluorotetrahydro-1H-pyrrolizin-7a(5H)- yl)methoxy)-4-(1-isopropyl-2-(1H-1,2,4-triazole- 1-carbonyl)-2,6-diazaspiro[3.5]nonan-6- yl)quinazolin-7-yl)-7-fluorobenzo[b]thiophene-3- carbonitrile | 791.4 |
| 182 | 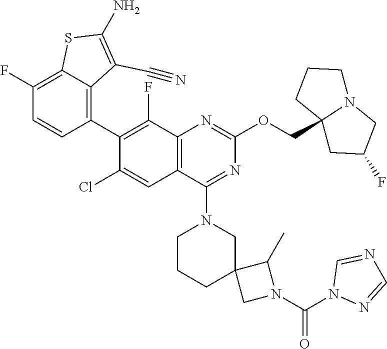 [SMILES: CC1N(C(=O)n2cncn2)CC12CCCN(c1nc(OC[C@@]34CCCN3C[C@H](F)C4)nc3c(F)c(-c4ccc(F)c5sc(N)c(C#N)c45)c(Cl)cc13)C2] | 2-amino-4-(6-chloro-8-fluoro-2-(((2R,7aS)-2- fluorotetrahydro-1H-pyrrolizin-7a(5H)- yl)methoxy)-4-(1-methyl-2-(1H-1,2,4-triazole-1- carbonyl)-2,6-diazaspiro[3.5]nonan-6- yl)quinazolin-7-yl)-7-fluorobenzo[b]thiophene-3- carbonitrile | 763.4 |
| 183^{†} | 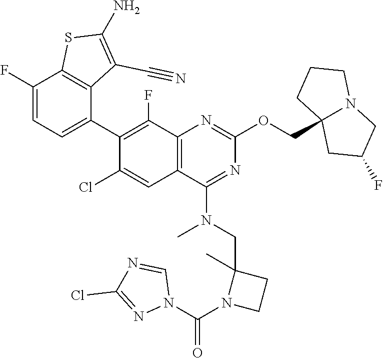 [SMILES: CN(CC1(C)CCN1C(=O)n1cnc(Cl)n1)c1nc(OC[C@@]23CCCN2C[C@H](F)C3)nc2c(F)c(-c3ccc(F)c4sc(N)c(C#N)c34)c(Cl)cc12] | 2-amino-4-(6-chloro-4-(((1-(3-chloro-1H-1,2,4- triazole-1-carbonyl)-2-methylazetidin-2- yl)methyl)(methyl)amino)-8-fluoro-2-(((2R,7aS)- 2-fluorotetrahydro-1H-pyrrolizin-7a(5H)- yl)methoxy)quinazolin-7-yl)-7- fluorobenzo[b]thiophene-3-carbonitrile (diastereomer 2) | 771.1 |
| 184^{†} | 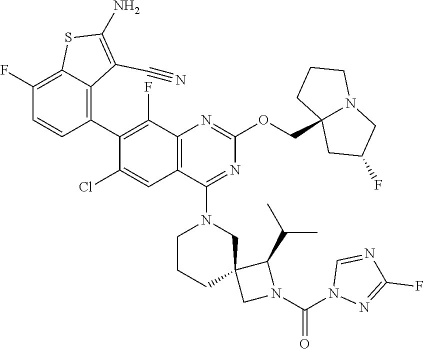 [SMILES: CC(C)[C@H]1N(C(=O)n2cnc(F)n2)C[C@]12CCCN(c1nc(OC[C@@]34CCCN3C[C@H](F)C4)nc3c(F)c(-c4ccc(F)c5sc(N)c(C#N)c45)c(Cl)cc13)C2] | 2-amino-4-(6-chloro-8-fluoro-4-((1R,4R)-2-(3- fluoro-1H-1,2,4-triazole-1-carbonyl)-1-isopropyl- 2,6-diazaspiro[3.5]nonan-6-yl)-2-(((2R,7aS)-2- fluorotetrahydro-1H-pyrrolizin-7a(5H)- yl)methoxy)quinazolin-7-yl)-7- fluorobenzo[b]thiophene-3-carbonitrile | 809.4 |
| 185^{†} | 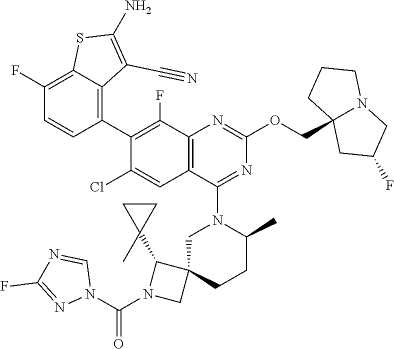 [SMILES: C[C@H]1CC[C@]2(CN(C(=O)n3cnc(F)n3)[C@@H]2C2(C)CC2)CN1c1nc(OC[C@@]23CCCN2C[C@H](F)C3)nc2c(F)c(-c3ccc(F)c4sc(N)c(C#N)c34)c(Cl)cc12] | 2-amino-4-(6-chloro-8-fluoro-4-((1R,4R,7S)-2-(3- fluoro-1H-1,2,4-triazole-1-carbonyl)-7-methyl-1- (1-methylcyclopropyl)-2,6-diazaspiro[3.5]nonan- 6-yl)-2-(((2R,7aS)-2-fluorotetrahydro-1H- pyrrolizin-7a(5H)-yl)methoxy)quinazolin-7-yl)-7- fluorobenzo[b]thiophene-3-carbonitrile | 835.3 |
| 186^{†} | 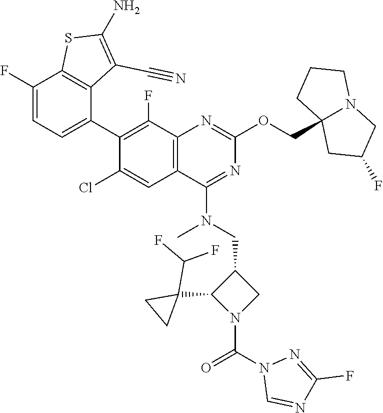 [SMILES: CN(C[C@@H]1CN(C(=O)n2cnc(F)n2)[C@@H]1C1(C(F)F)CC1)c1nc(OC[C@@]23CCCN2C[C@H](F)C3)nc2c(F)c(-c3ccc(F)c4sc(N)c(C#N)c34)c(Cl)cc12] | 2-amino-4-(6-chloro-4-((((2S,3R)-2-(1- (difluoromethyl)cyclopropyl)-1-(3-fluoro-1H- 1,2,4-triazole-1-carbonyl)azetidin-3- yl)methyl)(methyl)amino)-8-fluoro-2-(((2R,7aS)- 2-fluorotetrahydro-1H-pyrrolizin-7a(5H)- yl)methoxy)quinazolin-7-yl)-7- fluorobenzo[b]thiophene-3-carbonitrile | 831.3 |
| 187^{†} | 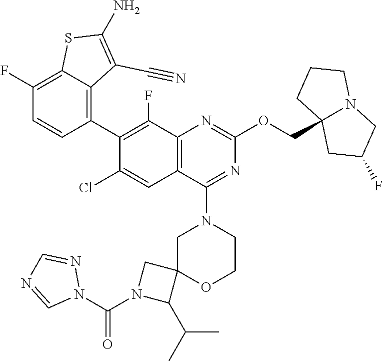 [SMILES: CC(C)C1N(C(=O)n2cncn2)CC12CN(c1nc(OC[C@@]34CCCN3C[C@H](F)C4)nc3c(F)c(-c4ccc(F)c5sc(N)c(C#N)c45)c(Cl)cc13)CCO2] | 2-amino-4-(6-chloro-8-fluoro-2-(((2R,7aS)-2- fluorotetrahydro-1H-pyrrolizin-7a(5H)- yl)methoxy)-4-(1-isopropyl-2-(1H-1,2,4-triazole- 1-carbonyl)-5-oxa-2,8-diazaspiro[3.5]nonan-8- yl)quinazolin-7-yl)-7-fluorobenzo[b]thiophene-3- carbonitrile | 793.4 |
| 188^{†} | 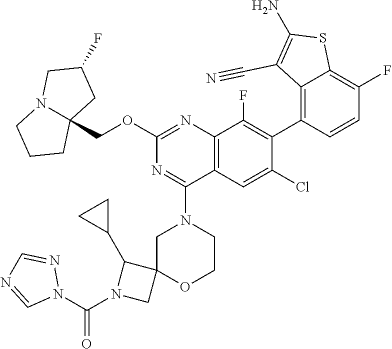 [SMILES: N#Cc1c(N)sc2c(F)ccc(-c3c(Cl)cc4c(N5CCOC6(C5)CN(C(=O)n5cncn5)C6C5CC5)nc(OC[C@@]56CCCN5C[C@H](F)C6)nc4c3F)c12] | 2-amino-4-(6-chloro-4-(1-cyclopropyl-2-(1H- 1,2,4-triazole-1-carbonyl)-5-oxa-2,8- diazaspiro[3.5]nonan-8-yl)-8-fluoro-2-(((2R,7aS)- 2-fluorotetrahydro-1H-pyrrolizin-7a(5H)- yl)methoxy)quinazolin-7-yl)-7- fluorobenzo[b]thiophene-3-carbonitrile | 791.2 |
| 189^{†} | 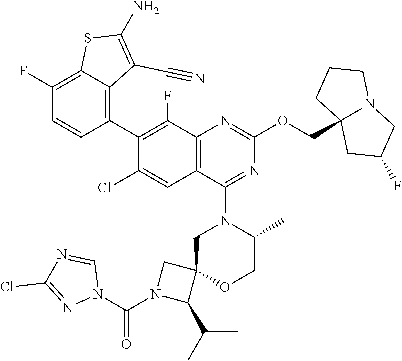 [SMILES: CC(C)[C@H]1N(C(=O)n2cnc(Cl)n2)C[C@@]12CN(c1nc(OC[C@@]34CCCN3C[C@H](F)C4)nc3c(F)c(-c4ccc(F)c5sc(N)c(C#N)c45)c(Cl)cc13)[C@H](C)CO2] | 2-amino-4-(6-chloro-4-((1R,4S,7R)-2-(3-chloro- 1H-1,2,4-triazole-1-carbonyl)-1-isopropyl-7- methyl-5-oxa-2,8-diazaspiro[3.5]nonan-8-yl)-8- fluoro-2-(((2R,7aS)-2-fluorotetrahydro-1H- pyrrolizin-7a(5H)-yl)methoxy)quinazolin-7-yl)-7- fluorobenzo[b]thiophene-3-carbonitrile | 841.7 |
| 190^{†} | 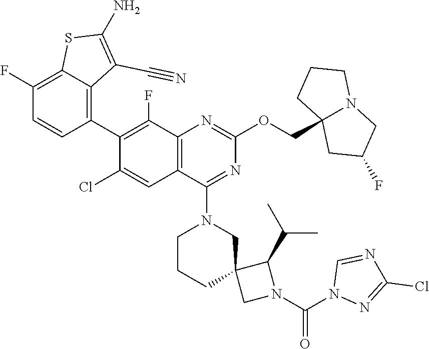 [SMILES: CC(C)[C@H]1N(C(=O)n2cnc(Cl)n2)C[C@]12CCCN(c1nc(OC[C@@]34CCCN3C[C@H](F)C4)nc3c(F)c(-c4ccc(F)c5sc(N)c(C#N)c45)c(Cl)cc13)C2] | 2-amino-4-(6-chloro-4-((1R,4R)-2-(3-chloro-1H- 1,2,4-triazole-1-carbonyl)-1-isopropyl-2,6- diazaspiro[3.5]nonan-6-yl)-8-fluoro-2-(((2R,7aS)- 2-fluorotetrahydro-1H-pyrrolizin-7a(5H)- yl)methoxy)quinazolin-7-yl)-7- fluorobenzo[b]thiophene-3-carbonitrile | 825.4 |
| 191^{†} | 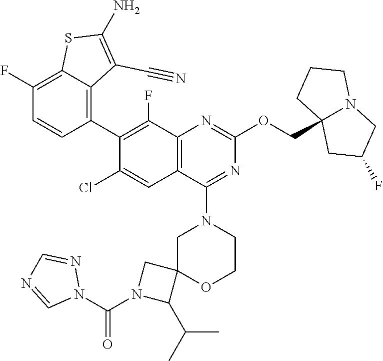 [SMILES: CC(C)C1N(C(=O)n2cncn2)CC12CN(c1nc(OC[C@@]34CCCN3C[C@H](F)C4)nc3c(F)c(-c4ccc(F)c5sc(N)c(C#N)c45)c(Cl)cc13)CCO2] | 2-amino-4-(6-chloro-8-fluoro-2-(((2R,7aS)-2- fluorotetrahydro-1H-pyrrolizin-7a(5H)- yl)methoxy)-4-(1-isopropyl-2-(1H-1,2,4-triazole- 1-carbonyl)-5-oxa-2,8-diazaspiro[3.5]nonan-8- yl)quinazolin-7-yl)-7-fluorobenzo[b]thiophene-3- carbonitrile | 793.4 |
| 192^{†} | 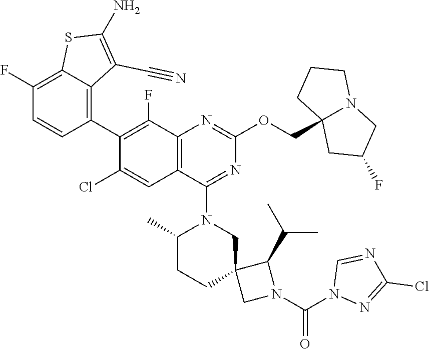 [SMILES: CC(C)[C@H]1N(C(=O)n2cnc(Cl)n2)C[C@]12CC[C@H](C)N(c1nc(OC[C@@]34CCCN3C[C@H](F)C4)nc3c(F)c(-c4ccc(F)c5sc(N)c(C#N)c45)c(Cl)cc13)C2] | 2-amino-4-(6-chloro-4-((1R,4R,7S)-2-(3-chloro- 1H-1,2,4-triazole-1-carbonyl)-1-isopropyl-7- methyl-2,6-diazaspiro[3.5]nonan-6-yl)-8-fluoro-2- (((2R,7aS)-2-fluorotetrahydro-1H-pyrrolizin- 7a(5H)-yl)methoxy)quinazolin-7-yl)-7- fluorobenzo[b]thiophene-3-carbonitrile | 839.4 |
| 193^{†} | 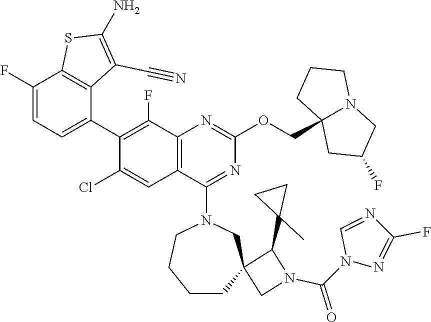 [SMILES: CC1([C@H]2N(C(=O)n3cnc(F)n3)C[C@]23CCCCN(c2nc(OC[C@@]45CCCN4C[C@H](F)C5)nc4c(F)c(-c5ccc(F)c6sc(N)c(C#N)c56)c(Cl)cc24)C3)CC1] | 2-amino-4-(6-chloro-8-fluoro-4-((1R,4R)-2-(3- fluoro-1H-1,2,4-triazole-1-carbonyl)-1-(1- methylcyclopropyl)-2,6-diazaspiro[3.6]decan-6- yl)-2-(((2R,7aS)-2-fluorotetrahydro-1H-pyrrolizin- 7a(5H)-yl)methoxy)quinazolin-7-yl)-7- fluorobenzo[b]thiophene-3-carbonitrile | 835.5 |
| 194^{†} | 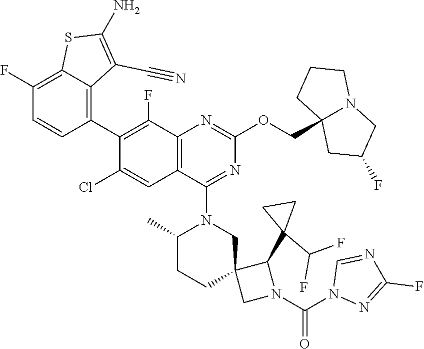 [SMILES: C[C@H]1CC[C@]2(CN(C(=O)n3cnc(F)n3)[C@@H]2C2(C(F)F)CC2)CN1c1nc(OC[C@@]23CCCN2C[C@H](F)C3)nc2c(F)c(-c3ccc(F)c4sc(N)c(C#N)c34)c(Cl)cc12] | 2-amino-4-(6-chloro-4-((1S,4R,7S)-1-(1- (difluoromethyl)cyclopropyl)-2-(3-fluoro-1H- 1,2,4-triazole-1-carbonyl)-7-methyl-2,6- diazaspiro[3.5]nonan-6-yl)-8-fluoro-2-(((2R,7aS)- 2-fluorotetrahydro-1H-pyrrolizin-7a(5H)- yl)methoxy)quinazolin-7-yl)-7- fluorobenzo[b]thiophene-3-carbonitrile | 871.2 |
| 195^{†} | 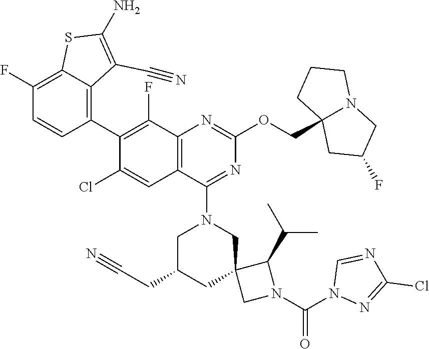 [SMILES: CC(C)[C@H]1N(C(=O)n2cnc(Cl)n2)C[C@]12C[C@H](CC#N)CN(c1nc(OC[C@@]34CCCN3C[C@H](F)C4)nc3c(F)c(-c4ccc(F)c5sc(N)c(C#N)c45)c(Cl)cc13)C2] | 2-amino-4-(6-chloro-4-((1R,4R,8R)-2-(3-chloro- 1H-1,2,4-triazole-1-carbonyl)-8-(cyanomethyl)-1- isopropyl-2,6-diazaspiro[3.5]nonan-6-yl)-8-fluoro- 2-(((2R,7aS)-2-fluorotetrahydro-1H-pyrrolizin- 7a(5H)-yl)methoxy)quinazolin-7-yl)-7- fluorobenzo[b]thiophene-3-carbonitrile | 864.3 |
| 196^{†} | 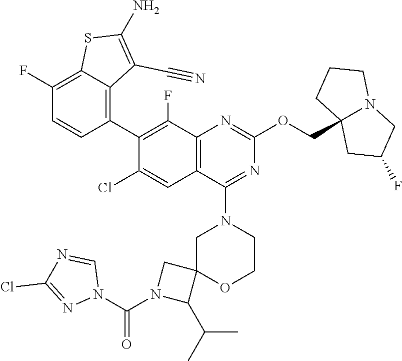 [SMILES: CC(C)C1N(C(=O)n2cnc(Cl)n2)CC12CN(c1nc(OC[C@@]34CCCN3C[C@H](F)C4)nc3c(F)c(-c4ccc(F)c5sc(N)c(C#N)c45)c(Cl)cc13)CCO2] | 2-amino-4-(6-chloro-4-(2-(3-chloro-1H-1,2,4- triazole-1-carbonyl)-1-isopropyl-5-oxa-2,8- diazaspiro[3.5]nonan-8-yl)-8-fluoro-2-(((2R,7aS)- 2-fluorotetrahydro-1H-pyrrolizin-7a(5H)- yl)methoxy)quinazolin-7-yl)-7- fluorobenzo[b]thiophene-3-carbonitrile (mixture of diastereomers) | 827.3 |
| 197^{†} | 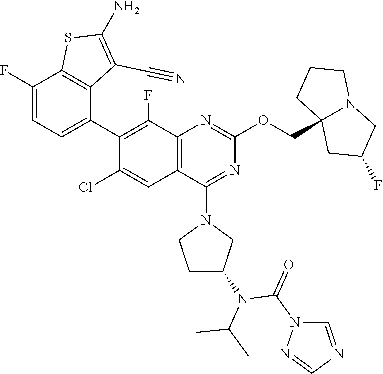 [SMILES: CC(C)N(C(=O)n1cncn1)[C@@H]1CCN(c2nc(OC[C@@]34CCCN3C[C@H](F)C4)nc3c(F)c(-c4ccc(F)c5sc(N)c(C#N)c45)c(Cl)cc23)C1] | N-((3R)-1-(7-(2-amino-3-cyano-7- fluorobenzo[b]thiophen-4-yl)-6-chloro-8-fluoro-2- (((2R,7aS)-2-fluorotetrahydro-1H-pyrrolizin- 7a(5H)-yl)methoxy)quinazolin-4-yl)pyrrolidin-3- yl)-N-isopropyl-1H-1,2,4-triazole-1-carboxamide | 751.4 |
| 198^{†} | 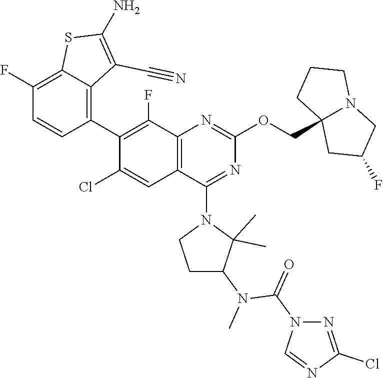 [SMILES: CN(C(=O)n1cnc(Cl)n1)C1CCN(c2nc(OC[C@@]34CCCN3C[C@H](F)C4)nc3c(F)c(-c4ccc(F)c5sc(N)c(C#N)c45)c(Cl)cc23)C1(C)C] | N-(1-(7-(2-amino-3-cyano-7- fluorobenzo[b]thiophen-4-yl)-6-chloro-8-fluoro-2- (((2R,7aS)-2-fluorotetrahydro-1H-pyrrolizin- 7a(5H)-yl)methoxy)quinazolin-4-yl)-2,2- dimethylpyrrolidin-3-yl)-3-chloro-N-methyl-1H- 1,2,4-triazole-1-carboxamide (diastereomer 2) | 785.4 |
| 199^{†} | 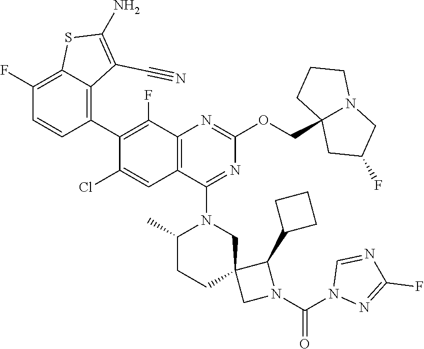 [SMILES: C[C@H]1CC[C@]2(CN(C(=O)n3cnc(F)n3)[C@@H]2C2CCC2)CN1c1nc(OC[C@@]23CCCN2C[C@H](F)C3)nc2c(F)c(-c3ccc(F)c4sc(N)c(C#N)c34)c(Cl)cc12] | 2-amino-4-(6-chloro-4-((1R,4R,7S)-1-cyclobutyl- 2-(3-fluoro-1H-1,2,4-triazole-1-carbonyl)-7- methyl-2,6-diazaspiro[3.5]nonan-6-yl)-8-fluoro-2- (((2R,7aS)-2-fluorotetrahydro-1H-pyrrolizin- 7a(5H)-yl)methoxy)quinazolin-7-yl)-7- fluorobenzo[b]thiophene-3-carbonitrile | 835.2 |
| 200^{†} | 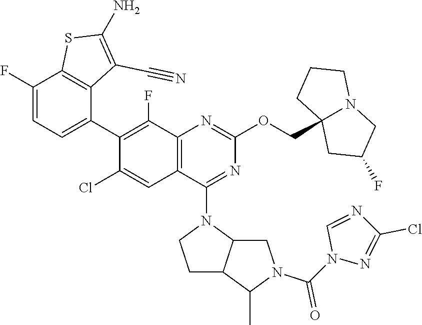 [SMILES: CC1C2CCN(c3nc(OC[C@@]45CCCN4C[C@H](F)C5)nc4c(F)c(-c5ccc(F)c6sc(N)c(C#N)c56)c(Cl)cc34)C2CN1C(=O)n1cnc(Cl)n1] | 2-amino-4-(6-chloro-4-(5-(3-chloro-1H-1,2,4- triazole-1-carbonyl)-4- methylhexahydropyrrolo[3,4-b]pyrrol-1(2H)-yl)-8- fluoro-2-(((2R,7aS)-2-fluorotetrahydro-1H- pyrrolizin-7a(5H)-yl)methoxy)quinazolin-7-yl)-7- fluorobenzo[b]thiophene-3-carbonitrile | 783.3 |
| 201^{†} | 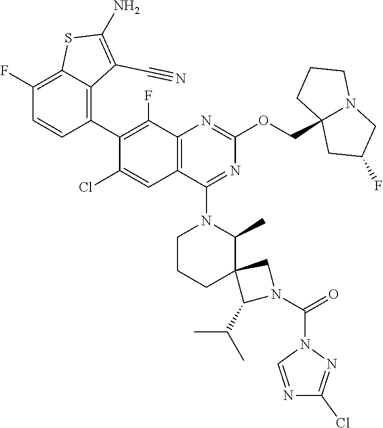 [SMILES: CC(C)[C@H]1N(C(=O)n2cnc(Cl)n2)C[C@]12CCCN(c1nc(OC[C@@]34CCCN3C[C@H](F)C4)nc3c(F)c(-c4ccc(F)c5sc(N)c(C#N)c45)c(Cl)cc13)[C@H]2C] | 2-amino-4-(6-chloro-4-((1R,4S,5S)-2-(3-chloro- 1H-1,2,4-triazole-1-carbonyl)-1-isopropyl-5- methyl-2,6-diazaspiro[3.5]nonan-6-yl)-8-fluoro-2- (((2R,7aS)-2-fluorotetrahydro-1H-pyrrolizin- 7a(5H)-yl)methoxy)quinazolin-7-yl)-7- fluorobenzo[b]thiophene-3-carbonitrile | 839.4 |
| 202^{†} | 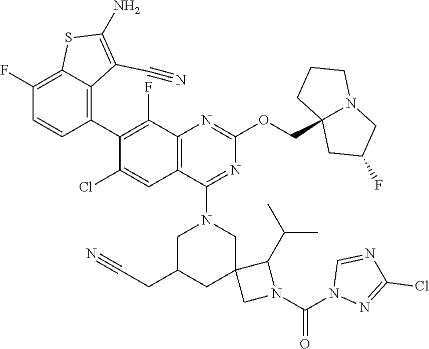 [SMILES: CC(C)C1N(C(=O)n2cnc(Cl)n2)CC12CC(CC#N)CN(c1nc(OC[C@@]34CCCN3C[C@H](F)C4)nc3c(F)c(-c4ccc(F)c5sc(N)c(C#N)c45)c(Cl)cc13)C2] | 2-amino-4-(6-chloro-4-(2-(3-chloro-1H-1,2,4- triazole-1-carbonyl)-8-(cyanomethyl)-1-isopropyl- 2,6-diazaspiro[3.5]nonan-6-yl)-8-fluoro-2- (((2R,7aS)-2-fluorotetrahydro-1H-pyrrolizin- 7a(5H)-yl)methoxy)quinazolin-7-yl)-7- fluorobenzo[b]thiophene-3-carbonitrile (diastereomer 2) | 864.4 |
| 203^{†} | 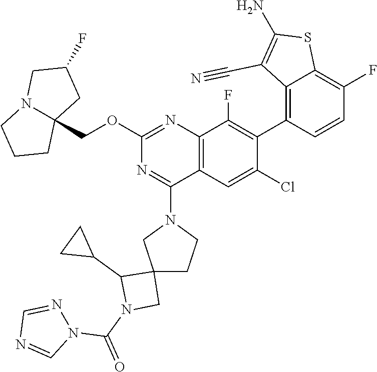 [SMILES: N#Cc1c(N)sc2c(F)ccc(-c3c(Cl)cc4c(N5CCC6(C5)CN(C(=O)n5cncn5)C6C5CC5)nc(OC[C@@]56CCCN5C[C@H](F)C6)nc4c3F)c12] | 2-amino-4-(6-chloro-4-(1-cyclopropyl-2-(1H- 1,2,4-triazole-1-carbonyl)-2,6- diazaspiro[3.4]octan-6-yl)-8-fluoro-2-(((2R,7aS)- 2-fluorotetrahydro-1H-pyrrolizin-7a(5H)- yl)methoxy)quinazolin-7-yl)-7- fluorobenzo[b]thiophene-3-carbonitrile (diastereomer 2) | 775.5 |
| 204^{†} | 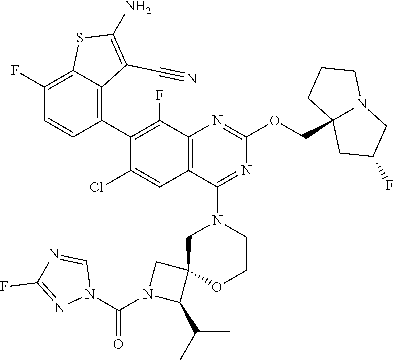 [SMILES: CC(C)[C@H]1N(C(=O)n2cnc(F)n2)C[C@@]12CN(c1nc(OC[C@@]34CCCN3C[C@H](F)C4)nc3c(F)c(-c4ccc(F)c5sc(N)c(C#N)c45)c(Cl)cc13)CCO2] | 2-amino-4-(6-chloro-8-fluoro-4-((1R,4S)-2-(3- fluoro-1H-1,2,4-triazole-1-carbonyl)-1-isopropyl- 5-oxa-2,8-diazaspiro[3.5]nonan-8-yl)-2- (((2R,7aS)-2-fluorotetrahydro-1H-pyrrolizin- 7a(5H)-yl)methoxy)quinazolin-7-yl)-7- fluorobenzo[b]thiophene-3-carbonitrile | 811.6 |
| 205^{†} | 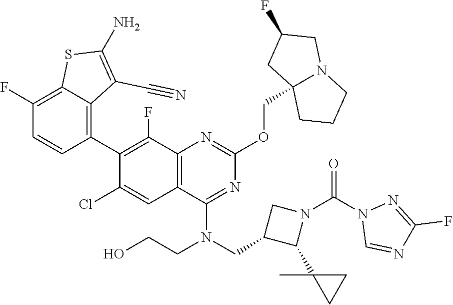 [SMILES: CC1([C@@H]2[C@H](CN(CCO)c3nc(OC[C@@]45CCCN4C[C@H](F)C5)nc4c(F)c(-c5ccc(F)c6sc(N)c(C#N)c56)c(Cl)cc34)CN2C(=O)n2cnc(F)n2)CC1] | 2-amino-4-(6-chloro-8-fluoro-4-((((2S,3R)-1-(3- fluoro-1H-1,2,4-triazole-1-carbonyl)-2-(1- methylcyclopropyl)azetidin-3-yl)methyl)(2- hydroxyethyl)amino)-2-(((2R,7aS)-2- fluorotetrahydro-1H-pyrrolizin-7a(5H)- yl)methoxy)quinazolin-7-yl)-7- fluorobenzo[b]thiophene-3-carbonitrile | 825.2 |
| 206^{†} | 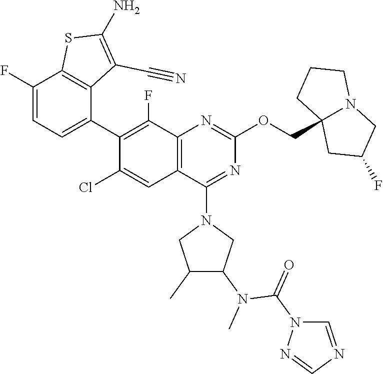 [SMILES: CC1CN(c2nc(OC[C@@]34CCCN3C[C@H](F)C4)nc3c(F)c(-c4ccc(F)c5sc(N)c(C#N)c45)c(Cl)cc23)CC1N(C)C(=O)n1cncn1] | N-(1-(7-(2-amino-3-cyano-7- fluorobenzo[b]thiophen-4-yl)-6-chloro-8-fluoro-2- (((2R,7aS)-2-fluorotetrahydro-1H-pyrrolizin- 7a(5H)-yl)methoxy)quinazolin-4-yl)-4- methylpyrrolidin-3-yl)-N-methyl-1H-1,2,4 triazole-1-carboxamide | 737.4 |
| 207^{†} | 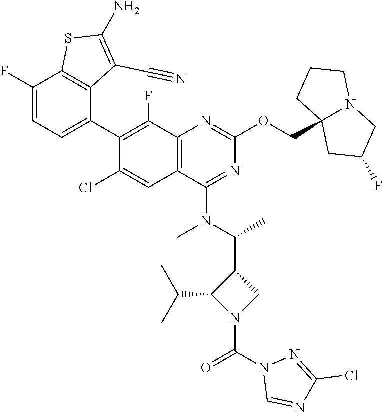 [SMILES: CC(C)[C@@H]1[C@H]([C@@H](C)N(C)c2nc(OC[C@@]34CCCN3C[C@H](F)C4)nc3c(F)c(-c4ccc(F)c5sc(N)c(C#N)c45)c(Cl)cc23)CN1C(=O)n1cnc(Cl)n1] | 2-amino-4-(6-chloro-4-(((R)-1-((2R,3S)-1-(3- chloro-1H-1,2,4-triazole-1-carbonyl)-2- isopropylazetidin-3-yl)ethyl)(methyl)amino)-8- fluoro-2-(((2R,7aS)-2-fluorotetrahydro-1H- pyrrolizin-7a(5H)-yl)methoxy)quinazolin-7-yl)-7- fluorobenzo[b]thiophene-3-carbonitrile | 813.6 |
| 208^{†} | 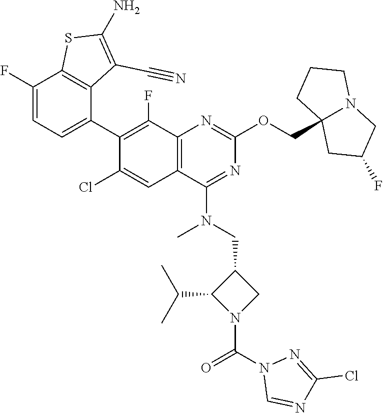 [SMILES: CC(C)[C@@H]1[C@H](CN(C)c2nc(OC[C@@]34CCCN3C[C@H](F)C4)nc3c(F)c(-c4ccc(F)c5sc(N)c(C#N)c45)c(Cl)cc23)CN1C(=O)n1cnc(Cl)n1] | 2-amino-4-(6-chloro-4-((((2R,3R)-1-(3-chloro-1H- 1,2,4-triazole-1-carbonyl)-2-isopropylazetidin-3- yl)methyl)(methyl)amino)-8-fluoro-2-(((2R,7aS)- 2-fluorotetrahydro-1H-pyrrolizin-7a(5H)- yl)methoxy)quinazolin-7-yl)-7- fluorobenzo[b]thiophene-3-carbonitrile | 799.4 |
| 209^{†} | 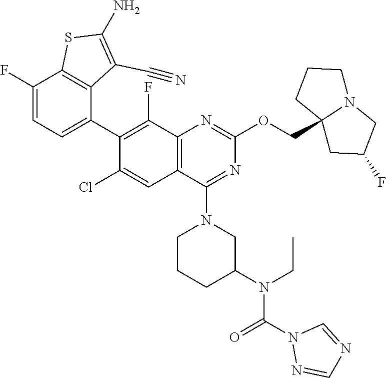 [SMILES: CCN(C(=O)n1cncn1)C1CCCN(c2nc(OC[C@@]34CCCN3C[C@H](F)C4)nc3c(F)c(-c4ccc(F)c5sc(N)c(C#N)c45)c(Cl)cc23)C1] | N-(1-(7-(2-amino-3-cyano-7- fluorobenzo[b]thiophen-4-yl)-6-chloro-8-fluoro-2- (((2R,7aS)-2-fluorotetrahydro-1H-pyrrolizin- 7a(5H)-yl)methoxy)quinazolin-4-yl)piperidin-3- yl)-N-ethyl-1H-1,2,4-triazole-1-carboxamide | 751.5 |
| 210^{†} | 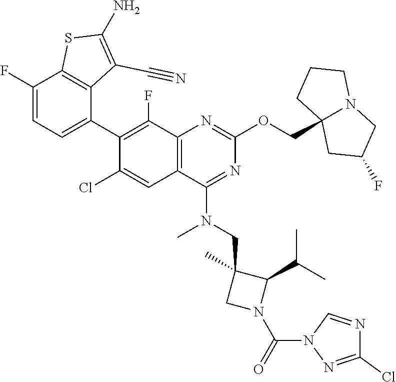 | 2-amino-4-(6-chloro-4-((((2R,3R)-1-(3-chloro-1H- 1,2,4-triazole-1-carbonyl)-2-isopropyl-3- methylazetidin-3-yl)methyl)(methyl)amino)-8- fluoro-2-(((2R,7aS)-2-fluorotetrahydro-1H- pyrrolizin-7a(5H)-yl)methoxy)quinazolin-7-yl)-7- fluorobenzo[b]thiophene-3-carbonitrile | 813.7 |
| 211^{†} | 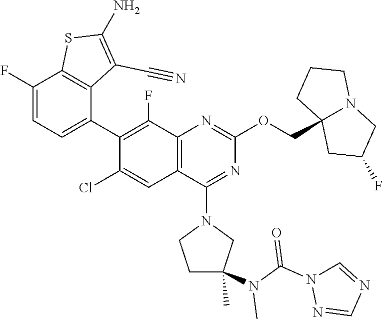 [SMILES: CN(C(=O)n1cncn1)[C@@]1(C)CCN(c2nc(OC[C@@]34CCCN3C[C@H](F)C4)nc3c(F)c(-c4ccc(F)c5sc(N)c(C#N)c45)c(Cl)cc23)C1] | N-((3S)-1-(7-(2-amino-3-cyano-7- fluorobenzo[b]thiophen-4-yl)-6-chloro-8-fluoro-2- (((2R,7aS)-2-fluorotetrahydro-1H-pyrrolizin- 7a(5H)-yl)methoxy)quinazolin-4-yl)-3- methylpyrrolidin-3-yl)-N-methyl-1H-1,2,4- triazole-1-carboxamide | 737.2 |
| 212^{†} | 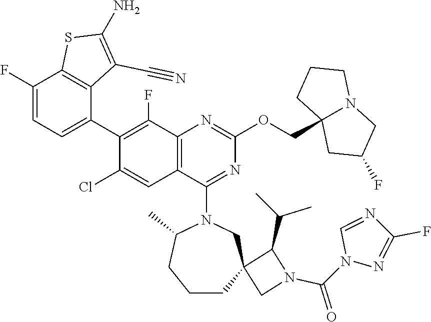 [SMILES: CC(C)[C@H]1N(C(=O)n2cnc(F)n2)C[C@]12CCC[C@H](C)N(c1nc(OC[C@@]34CCCN3C[C@H](F)C4)nc3c(F)c(-c4ccc(F)c5sc(N)c(C#N)c45)c(Cl)cc13)C2] | 2-amino-4-(6-chloro-8-fluoro-4-((1R,4R,7S)-2-(3- fluoro-1H-1,2,4-triazole-1-carbonyl)-1-isopropyl- 7-methyl-2,6-diazaspiro[3.6]decan-6-yl)-2- (((2R,7aS)-2-fluorotetrahydro-1H-pyrrolizin- 7a(5H)-yl)methoxy)quinazolin-7-yl)-7- fluorobenzo[b]thiophene-3-carbonitrile | 837.2 |
| 213^{†} | 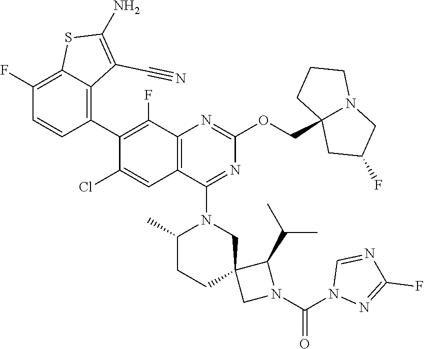 [SMILES: CC(C)[C@H]1N(C(=O)n2cnc(F)n2)C[C@]12CC[C@H](C)N(c1nc(OC[C@@]34CCCN3C[C@H](F)C4)nc3c(F)c(-c4ccc(F)c5sc(N)c(C#N)c45)c(Cl)cc13)C2] | 2-amino-4-(6-chloro-8-fluoro-4-((1R,4R,7S)-2-(3- fluoro-1H-1,2,4-triazole-1-carbonyl)-1-isopropyl- 7-methyl-2,6-diazaspiro[3.5]nonan-6-yl)-2- (((2R,7aS)-2-fluorotetrahydro-1H-pyrrolizin- 7a(5H)-yl)methoxy)quinazolin-7-yl)-7- fluorobenzo[b]thiophene-3-carbonitrile | 823.4 |
| 214^{†} | 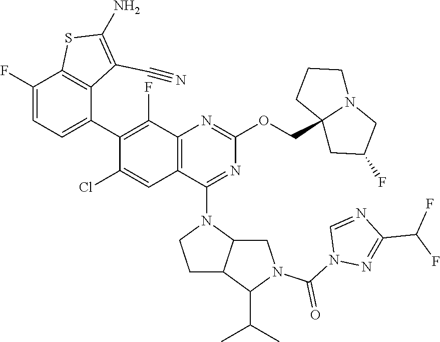 [SMILES: CC(C)C1C2CCN(c3nc(OC[C@@]45CCCN4C[C@H](F)C5)nc4c(F)c(-c5ccc(F)c6sc(N)c(C#N)c56)c(Cl)cc34)C2CN1C(=O)n1cnc(C(F)F)n1] | 2-amino-4-(6-chloro-4-(5-(3-(difluoromethyl)-1H- 1,2,4-triazole-1-carbonyl)-4- isopropylhexahydropyrrolo[3,4-b]pyrrol-1(2H)- yl)-8-fluoro-2-(((2R,7aS)-2-fluorotetrahydro-1H- pyrrolizin-7a(5H)-yl)methoxy)quinazolin-7-yl)-7- fluorobenzo[b]thiophene-3-carbonitrile (diastereomer 2) | 827.3 |
| 215^{†} | 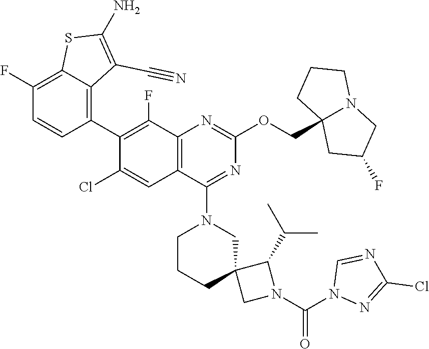 [SMILES: CC(C)[C@@H]1N(C(=O)n2cnc(Cl)n2)C[C@@]12CCCN(c1nc(OC[C@@]34CCCN3C[C@H](F)C4)nc3c(F)c(-c4ccc(F)c5sc(N)c(C#N)c45)c(Cl)cc13)C2] | 2-amino-4-(6-chloro-4-((1S,4S)-2-(3-chloro-1H- 1,2,4-triazole-1-carbonyl)-1-isopropyl-2,6- diazaspiro[3.5]nonan-6-yl)-8-fluoro-2-(((2R,7aS)- 2-fluorotetrahydro-1H-pyrrolizin-7a(5H)- yl)methoxy)quinazolin-7-yl)-7- fluorobenzo[b]thiophene-3-carbonitrile | 825.3 |
| 216^{†} | 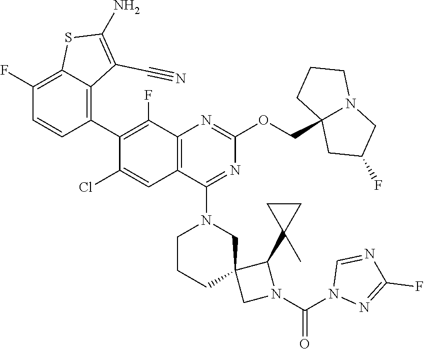 [SMILES: CC1([C@H]2N(C(=O)n3cnc(F)n3)C[C@]23CCCN(c2nc(OC[C@@]45CCCN4C[C@H](F)C5)nc4c(F)c(-c5ccc(F)c6sc(N)c(C#N)c56)c(Cl)cc24)C3)CC1] | 2-amino-4-(6-chloro-8-fluoro-4-((1R,4R)-2-(3- fluoro-1H-1,2,4-triazole-1-carbonyl)-1-(1- methylcyclopropyl)-2,6-diazaspiro[3.5]nonan-6- yl)-2-(((2R,7aS)-2-fluorotetrahydro-1H-pyrrolizin- 7a(5H)-yl)methoxy)quinazolin-7-yl)-7- fluorobenzo[b]thiophene-3-carbonitrile | 821.4 |
| 217^{†} | 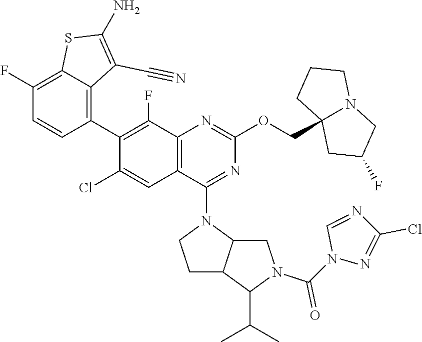 [SMILES: CC(C)C1C2CCN(c3nc(OC[C@@]45CCCN4C[C@H](F)C5)nc4c(F)c(-c5ccc(F)c6sc(N)c(C#N)c56)c(Cl)cc34)C2CN1C(=O)n1cnc(Cl)n1] | 2-amino-4-(6-chloro-4-(5-(3-chloro-1H-1,2,4- triazole-1-carbonyl)-4- isopropylhexahydropyrrolo[3,4-b]pyrrol-1(2H)- yl)-8-fluoro-2-(((2R,7aS)-2-fluorotetrahydro-1H- pyrrolizin-7a(5H)-yl)methoxy)quinazolin-7-yl)-7- fluorobenzo[b]thiophene-3-carbonitrile | 811.5 |
| 218^{†} | 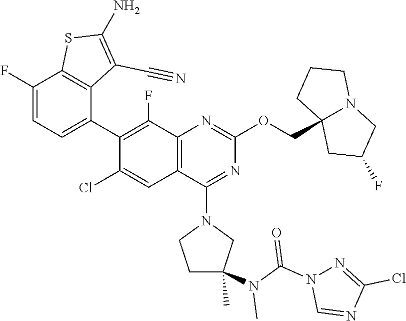 [SMILES: CN(C(=O)n1cnc(Cl)n1)[C@@]1(C)CCN(c2nc(OC[C@@]34CCCN3C[C@H](F)C4)nc3c(F)c(-c4ccc(F)c5sc(N)c(C#N)c45)c(Cl)cc23)C1] | N-((3S)-1-(7-(2-amino-3-cyano-7- fluorobenzo[b]thiophen-4-yl)-6-chloro-8-fluoro-2- (((2R,7aS)-2-fluorotetrahydro-1H-pyrrolizin- 7a(5H)-yl)methoxy)quinazolin-4-yl)-3- methylpyrrolidin-3-yl)-3-chloro-N-methyl-1H- 1,2,4-triazole-1-carboxamide | 771.2 |
| 219^{†} | 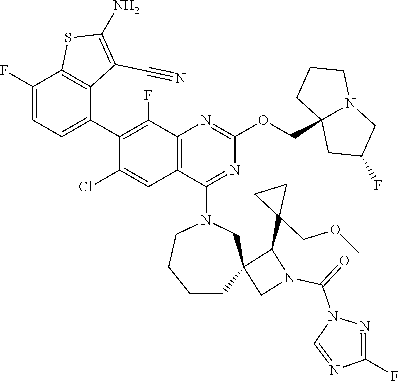 [SMILES: COCC1([C@H]2N(C(=O)n3cnc(F)n3)C[C@]23CCCCN(c2nc(OC[C@@]45CCCN4C[C@H](F)C5)nc4c(F)c(-c5ccc(F)c6sc(N)c(C#N)c56)c(Cl)cc24)C3)CC1] | 2-amino-4-(6-chloro-8-fluoro-4-((1S,4R)-2-(3- fluoro-1H-1,2,4-triazole-1-carbonyl)-1-(1- (methoxymethyl)cyclopropyl)-2,6- diazaspiro[3.6]decan-6-yl)-2-(((2R,7aS)-2- fluorotetrahydro-1H-pyrrolizin-7a(5H)- yl)methoxy)quinazolin-7-yl)-7- fluorobenzo[b]thiophene-3-carbonitrile | 865.5 |
| 220^{†} | 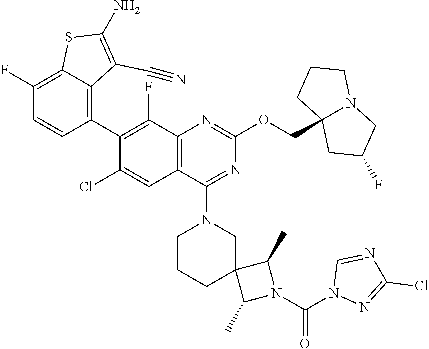 [SMILES: C[C@H]1N(C(=O)n2cnc(Cl)n2)[C@H](C)C12CCCN(c1nc(OC[C@@]34CCCN3C[C@H](F)C4)nc3c(F)c(-c4ccc(F)c5sc(N)c(C#N)c45)c(Cl)cc13)C2] | 2-amino-4-(6-chloro-4-((1R,3R)-2-(3-chloro-1H- 1,2,4-triazole-1-carbonyl)-1,3-dimethyl-2,6- diazaspiro[3.5]nonan-6-yl)-8-fluoro-2-(((2R,7aS)- 2-fluorotetrahydro-1H-pyrrolizin-7a(5H)- yl)methoxy)quinazolin-7-yl)-7- fluorobenzo[b]thiophene-3-carbonitrile | 811.2 |
| 221^{†} | 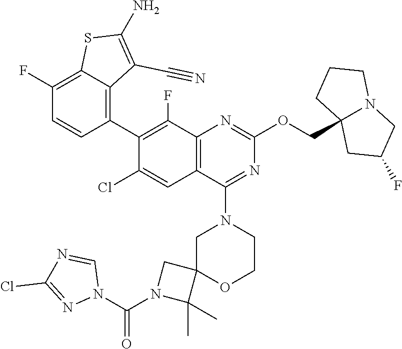 [SMILES: CC1(C)N(C(=O)n2cnc(Cl)n2)CC12CN(c1nc(OC[C@@]34CCCN3C[C@H](F)C4)nc3c(F)c(-c4ccc(F)c5sc(N)c(C#N)c45)c(Cl)cc13)CCO2] | 2-amino-4-(6-chloro-4-(2-(3-chloro-1H-1,2,4- triazole-1-carbonyl)-1,1-dimethyl-5-oxa-2,8- diazaspiro[3.5]nonan-8-yl)-8-fluoro-2-(((2R,7aS)- 2-fluorotetrahydro-1H-pyrrolizin-7a(5H)- yl)methoxy)quinazolin-7-yl)-7- fluorobenzo[b]thiophene-3-carbonitrile | 813.4 |
| 222^{†} | 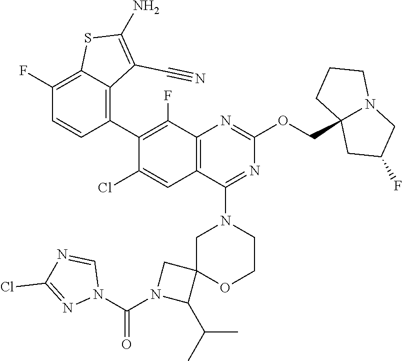 [SMILES: CC(C)C1N(C(=O)n2cnc(Cl)n2)CC12CN(c1nc(OC[C@@]34CCCN3C[C@H](F)C4)nc3c(F)c(-c4ccc(F)c5sc(N)c(C#N)c45)c(Cl)cc13)CCO2] | 2-amino-4-(6-chloro-4-(2-(3-chloro-1H-1,2,4- triazole-1-carbonyl)-1-isopropyl-5-oxa-2,8- diazaspiro[3.5]nonan-8-yl)-8-fluoro-2-(((2R,7aS)- 2-fluorotetrahydro-1H-pyrrolizin-7a(5H)- yl)methoxy)quinazolin-7-yl)-7- fluorobenzo[b]thiophene-3-carbonitrile (mixture of diastereomers) | 827.5 |
| 223 | 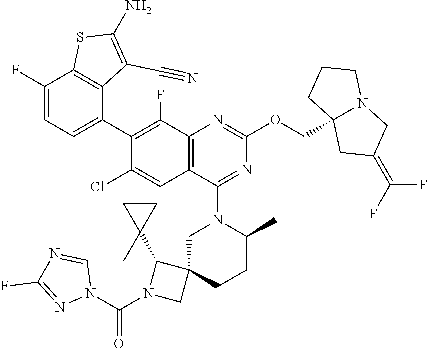 [SMILES: C[C@H]1CC[C@]2(CN(C(=O)n3cnc(F)n3)[C@@H]2C2(C)CC2)CN1c1nc(OC[C@]23CCCN2CC(=C(F)F)C3)nc2c(F)c(-c3ccc(F)c4sc(N)c(C#N)c34)c(Cl)cc12] | 2-amino-4-(6-chloro-2-(((R)-2- (difluoromethylene)tetrahydro-1H-pyrrolizin- 7a(5H)-yl)methoxy)-8-fluoro-4-((1R,4R,7S)-2-(3- fluoro-1H-1,2,4-triazole-1-carbonyl)-7-methyl-1- (1-methylcyclopropyl)-2,6-diazaspiro[3.5]nonan- 6-yl)quinazolin-7-yl)-7-fluorobenzo[b]thiophene- 3-carbonitrile ((R)-tetrahydro-1H-pyrrolizin-7a(5H)-yl stereochemistry tentatively assigned) | 865.2 |
| 224^{†} | 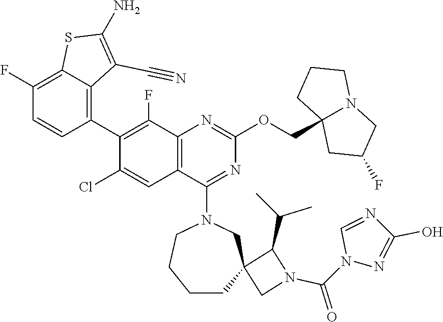 [SMILES: CC(C)[C@H]1N(C(=O)n2cnc(O)n2)C[C@]12CCCCN(c1nc(OC[C@@]34CCCN3C[C@H](F)C4)nc3c(F)c(-c4ccc(F)c5sc(N)c(C#N)c45)c(Cl)cc13)C2] | 2-amino-4-(6-chloro-8-fluoro-2-(((2R,7aS)-2- fluorotetrahydro-1H-pyrrolizin-7a(5H)- yl)methoxy)-4-((1R,4R)-2-(3-hydroxy-1H-1,2,4- triazole-1-carbonyl)-1-isopropyl-2,6- diazaspiro[3.6]decan-6-yl)quinazolin-7-yl)-7- fluorobenzo[b]thiophene-3-carbonitrile | 821.3 |
| 225^{†} | 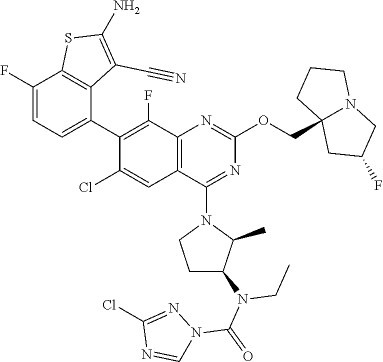 | N-((2S,3S)-1-(7-(2-amino-3-cyano-7- fluorobenzo[b]thiophen-4-yl)-6-chloro-8-fluoro-2- (((2R,7aS)-2-fluorotetrahydro-1H-pyrrolizin- 7a(5H)-yl)methoxy)quinazolin-4-yl)-2- methylpyrrolidin-3-yl)-3-chloro-N-ethyl-1H-1,2,4- triazole-1-carboxamide | 785.4 |
| 226^{†} | 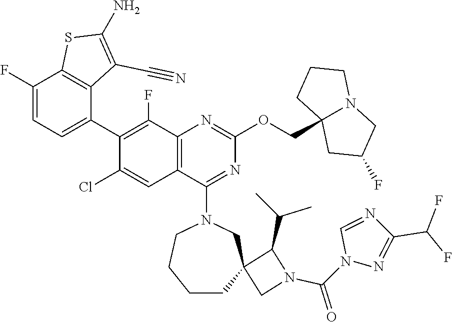 [SMILES: CC(C)[C@H]1N(C(=O)n2cnc(C(F)F)n2)C[C@]12CCCCN(c1nc(OC[C@@]34CCCN3C[C@H](F)C4)nc3c(F)c(-c4ccc(F)c5sc(N)c(C#N)c45)c(Cl)cc13)C2] | 2-amino-4-(6-chloro-4-((1R,4R)-2-(3- (difluoromethyl)-1H-1,2,4-triazole-1-carbonyl)-1- isopropyl-2,6-diazaspiro[3.6]decan-6-yl)-8-fluoro- 2-(((2R,7aS)-2-fluorotetrahydro-1H-pyrrolizin- 7a(5H)-yl)methoxy)quinazolin-7-yl)-7- fluorobenzo[b]thiophene-3-carbonitrile | 855.3 |
| 227^{†} | 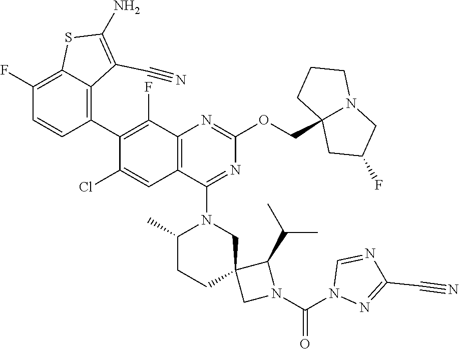 [SMILES: CC(C)[C@H]1N(C(=O)n2cnc(C#N)n2)C[C@]12CC[C@H](C)N(c1nc(OC[C@@]34CCCN3C[C@H](F)C4)nc3c(F)c(-c4ccc(F)c5sc(N)c(C#N)c45)c(Cl)cc13)C2] | 1-((1R,4R,7S)-6-(7-(2-amino-3-cyano-7- fluorobenzo[b]thiophen-4-yl)-6-chloro-8-fluoro-2- (((2R,7aS)-2-fluorotetrahydro-1H-pyrrolizin- 7a(5H)-yl)methoxy)quinazolin-4-yl)-1-isopropyl- 7-methyl-2,6-diazaspiro[3.5]nonane-2-carbonyl)- 1H-1,2,4-triazole-3-carbonitrile | 830.2 |
| 228^{†} | 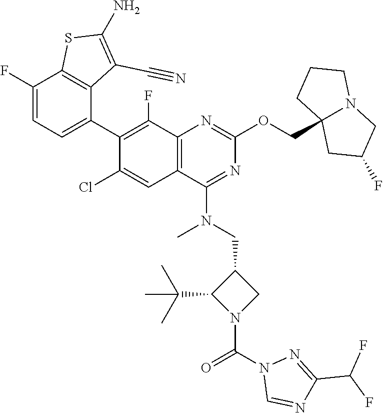 [SMILES: CN(C[C@@H]1CN(C(=O)n2cnc(C(F)F)n2)[C@@H]1C(C)(C)C)c1nc(OC[C@@]23CCCN2C[C@H](F)C3)nc2c(F)c(-c3ccc(F)c4sc(N)c(C#N)c34)c(Cl)cc12] | 2-amino-4-(4-((((2S,3R)-2-(tert-butyl)-1-(3- (difluoromethyl)-1H-1,2,4-triazole-1- carbonyl)azetidin-3-yl)methyl)(methyl)amino)-6- chloro-8-fluoro-2-(((2R,7aS)-2-fluorotetrahydro- 1H-pyrrolizin-7a(5H)-yl)methoxy)quinazolin-7- yl)-7-fluorobenzo[b]thiophene-3-carbonitrile | 829.3 |
| 229^{†} | 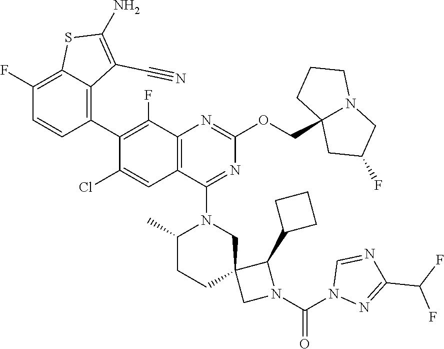 [SMILES: C[C@H]1CC[C@]2(CN(C(=O)n3cnc(C(F)F)n3)[C@@H]2C2CCC2)CN1c1nc(OC[C@@]23CCCN2C[C@H](F)C3)nc2c(F)c(-c3ccc(F)c4sc(N)c(C#N)c34)c(Cl)cc12] | 2-amino-4-(6-chloro-4-((1R,4R,7S)-1-cyclobutyl- 2-(3-(difluoromethyl)-1H-1,2,4-triazole-1- carbonyl)-7-methyl-2,6-diazaspiro[3.5]nonan-6- yl)-8-fluoro-2-(((2R,7aS)-2-fluorotetrahydro-1H- pyrrolizin-7a(5H)-yl)methoxy)quinazolin-7-yl)-7- fluorobenzo[b]thiophene-3-carbonitrile | 867.5 |
| 230^{†} | 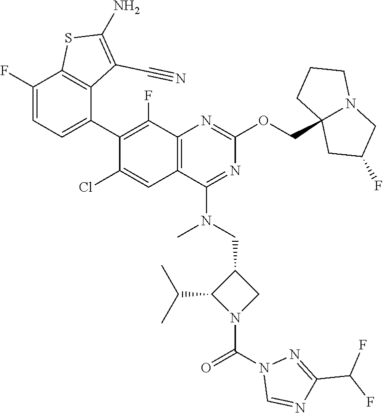 [SMILES: CC(C)[C@@H]1[C@H](CN(C)c2nc(OC[C@@]34CCCN3C[C@H](F)C4)nc3c(F)c(-c4ccc(F)c5sc(N)c(C#N)c45)c(Cl)cc23)CN1C(=O)n1cnc(C(F)F)n1] | 2-amino-4-(6-chloro-4-((((2R,3R)-1-(3- (difluoromethyl)-1H-1,2,4-triazole-1-carbonyl)-2- isopropylazetidin-3-yl)methyl)(methyl)amino)-8- fluoro-2-(((2R,7aS)-2-fluorotetrahydro-1H- pyrrolizin-7a(5H)-yl)methoxy)quinazolin-7-yl)-7- fluorobenzo[b]thiophene-3-carbonitrile | 815.3 |
| 231^{†} | 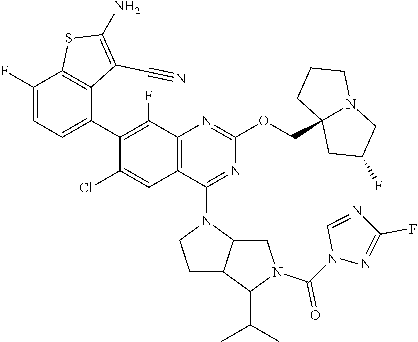 [SMILES: CC(C)C1C2CCN(c3nc(OC[C@@]45CCCN4C[C@H](F)C5)nc4c(F)c(-c5ccc(F)c6sc(N)c(C#N)c56)c(Cl)cc34)C2CN1C(=O)n1cnc(F)n1] | 2-amino-4-(6-chloro-8-fluoro-4-(5-(3-fluoro-1H- 1,2,4-triazole-1-carbonyl)-4- isopropylhexahydropyrrolo[3,4-b]pyrrol-1(2H)- yl)-2-(((2R,7aS)-2-fluorotetrahydro-1H-pyrrolizin- 7a(5H)-yl)methoxy)quinazolin-7-yl)-7- fluorobenzo[b]thiophene-3-carbonitrile (diastereomer 1) | 795.3 |
| 232^{†} | 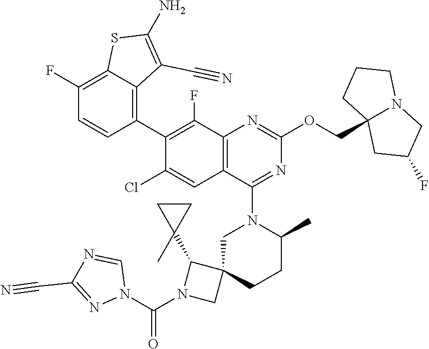 [SMILES: C[C@H]1CC[C@]2(CN(C(=O)n3cnc(C#N)n3)[C@@H]2C2(C)CC2)CN1c1nc(OC[C@@]23CCCN2C[C@H](F)C3)nc2c(F)c(-c3ccc(F)c4sc(N)c(C#N)c34)c(Cl)cc12] | 1-((1R,4R,7S)-6-(7-(2-amino-3-cyano-7- fluorobenzo[b]thiophen-4-yl)-6-chloro-8-fluoro-2- (((2R,7aS)-2-fluorotetrahydro-1H-pyrrolizin- 7a(5H)-yl)methoxy)quinazolin-4-yl)-7-methyl-1- (1-methylcyclopropyl)-2,6-diazaspiro[3.5]nonane- 2-carbonyl)-1H-1,2,4-triazole-3-carbonitrile | 842.2 |
| 233^{†} | 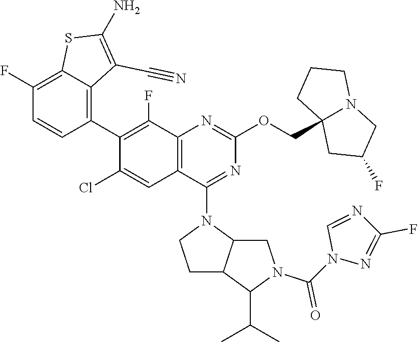 [SMILES: CC(C)C1C2CCN(c3nc(OC[C@@]45CCCN4C[C@H](F)C5)nc4c(F)c(-c5ccc(F)c6sc(N)c(C#N)c56)c(Cl)cc34)C2CN1C(=O)n1cnc(F)n1] | 2-amino-4-(6-chloro-8-fluoro-4-(5-(3-fluoro-1H- 1,2,4-triazole-1-carbonyl)-4- isopropylhexahydropyrrolo[3,4-b]pyrrol-1(2H)- yl)-2-(((2R,7aS)-2-fluorotetrahydro-1H-pyrrolizin- 7a(5H)-yl)methoxy)quinazolin-7-yl)-7- fluorobenzo[b]thiophene-3-carbonitrile (diastereomer 2) | 795.2 |
| 234^{†} | 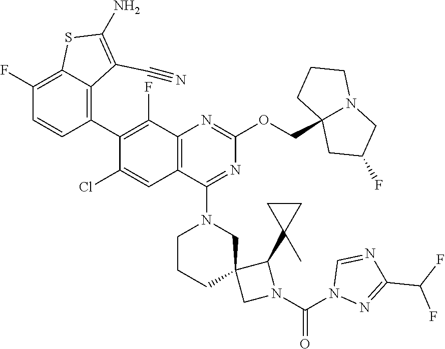 [SMILES: CC1([C@H]2N(C(=O)n3cnc(C(F)F)n3)C[C@]23CCCN(c2nc(OC[C@@]45CCCN4C[C@H](F)C5)nc4c(F)c(-c5ccc(F)c6sc(N)c(C#N)c56)c(Cl)cc24)C3)CC1] | 2-amino-4-(6-chloro-4-((1R,4R)-2-(3- (difluoromethyl)-1H-1,2,4-triazole-1-carbonyl)-1- (1-methylcyclopropyl)-2,6-diazaspiro[3.5]nonan- 6-yl)-8-fluoro-2-(((2R,7aS)-2-fluorotetrahydro- 1H-pyrrolizin-7a(5H)-yl)methoxy)quinazolin-7- yl)-7-fluorobenzo[b]thiophene-3-carbonitrile | 853.3 |
| 235^{†} | 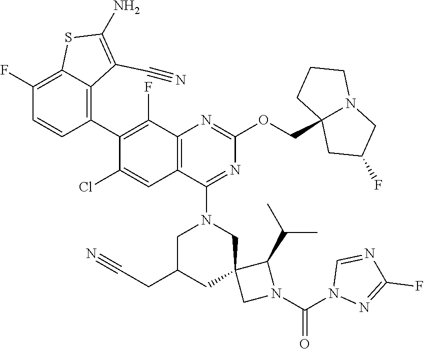 | 2-amino-4-(6-chloro-4-((1R,4R)-8-(cyanomethyl)- 2-(3-fluoro-1H-1,2,4-triazole-1-carbonyl)-1- isopropyl-2,6-diazaspiro[3.5]nonan-6-yl)-8-fluoro- 2-(((2R,7aS)-2-fluorotetrahydro-1H-pyrrolizin- 7a(5H)-yl)methoxy)quinazolin-7-yl)-7- fluorobenzo[b]thiophene-3-carbonitrile | 848.3 |
| 236^{†} | 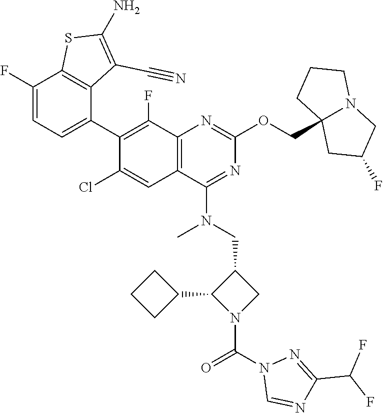 [SMILES: CN(C[C@@H]1CN(C(=O)n2cnc(C(F)F)n2)[C@@H]1C1CCC1)c1nc(OC[C@@]23CCCN2C[C@H](F)C3)nc2c(F)c(-c3ccc(F)c4sc(N)c(C#N)c34)c(Cl)cc12] | 2-amino-4-(6-chloro-4-((((2R,3R)-2-cyclobutyl-1- (3-(difluoromethyl)-1H-1,2,4-triazole-1- carbonyl)azetidin-3-yl)methyl)(methyl)amino)-8- fluoro-2-(((2R,7aS)-2-fluorotetrahydro-1H- pyrrolizin-7a(5H)-yl)methoxy)quinazolin-7-yl)-7- fluorobenzo[b]thiophene-3-carbonitrile | 827.2 |
| 237^{†} | 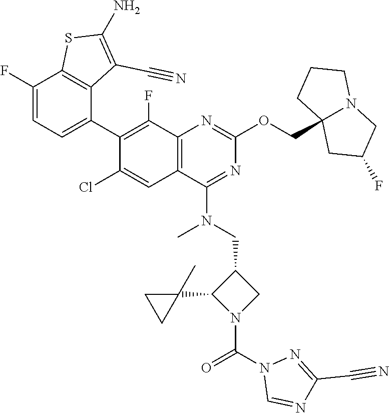 [SMILES: CN(C[C@@H]1CN(C(=O)n2cnc(C#N)n2)[C@@H]1C1(C)CC1)c1nc(OC[C@@]23CCCN2C[C@H](F)C3)nc2c(F)c(-c3ccc(F)c4sc(N)c(C#N)c34)c(Cl)cc12] | 1-((2S,3R)-3-(((7-(2-amino-3-cyano-7- fluorobenzo[b]thiophen-4-yl)-6-chloro-8-fluoro-2- (((2R,7aS)-2-fluorotetrahydro-1H-pyrrolizin- 7a(5H)-yl)methoxy)quinazolin-4- yl)(methyl)amino)methyl)-2-(1- methylcyclopropyl)azetidine-1-carbonyl)-1H- 1,2,4-triazole-3-carbonitrile | 802.2 |

Compounds of Table 1 are depicted with flat, wedged, and/or hashed wedged bonds. It is understood that compounds depicted in Table 1 encompass all possible stereoisomers, including atropisomers, of the compounds of Table 1. In some instances, the relative stereochemistry at one or more stereocenters of a compound has been determined; in some instances, the absolute stereochemistry has been determined. In some instances, a single compound number represents a mixture of stereoisomers, including atropisomers. In some instances, a single compound number represents a single stereoisomer, such as a single atropisomer. As such, it is understood that if two or more compound numbers in Table 1 are provided with the same depicted structure, then different stereoisomers or mixtures of stereoisomers of the depicted structure are represented by each compound number. † Compound provided as a substantially pure single atropisomer (R).

In some embodiments, the compounds of the present disclosure exhibit one or more functional characteristics disclosed herein. For example, a subject compound binds to a Ras protein, Kras protein or a mutant form thereof. In some embodiments, a subject compound binds specifically and also inhibits a Ras protein, Kras protein or a mutant form thereof. In some embodiments, a subject compound selectively inhibits a Kras mutant relative to a wildtype Kras. In some embodiments, the IC50 of a subject compound for a Kras mutant (e.g., including G12S) is less than about 5 μM, less than about 1 μM, less than about 500 nM, less than 250 nM, less than 100 nM, less than 50 nM, or even less, as measured in an in vitro assay known in the art or exemplified herein. In some embodiments, a subject compound covalently binds to a Kras mutant (e.g., KrasG12S and/or KrasG12C).

In some embodiments, a compound of the present disclosure is capable of reducing Ras signaling output. Such reduction may be evidenced by one or more of the following: (i) an increase in steady state level of GDP-bound Ras protein; (ii) a reduction in steady state level of GTP-bound Ras protein; (iii) a reduction of phosphorylated AKTs473, (iv) a reduction of phosphorylated ERKT202/y204, (v) a reduction of phosphorylated S6S235/236, and (vi) reduction (e.g., inhibition) of cell growth of Ras-driven tumor cells (e.g., those derived from a tumor cell line disclosed herein). In some cases, the reduction in Ras signaling output can be evidenced by two, three, four, five, or all of (i)-(vi) above.

It shall be understood that different aspects of the disclosure can be appreciated individually, collectively, or in combination with each other. Various aspects described herein may be applied to any of the particular applications disclosed herein. The compositions of matter, including compounds of any formulae disclosed in the compound section, of the present disclosure may be utilized in the method section, including methods of use and production disclosed herein, or vice versa.

### Methods

The compounds described herein, or a pharmaceutically acceptable salt or solvate thereof, are Ras inhibitors capable of inhibiting a Ras protein, such as wild-type Ras or a Ras mutant protein (e.g., G12S, G12C, G12D, G12V, G13C, and/or G13D) from K-Ras, H-Ras, or N-Ras. Compounds, including pharmaceutically acceptable salts or solvates thereof, disclosed herein have a wide range of applications in therapeutics, diagnostics, and other biomedical research.

In certain aspects, the present disclosure provides a method of treating cancer in a subject in need thereof, comprising administering to the subject a therapeutically effective amount of a compound described herein, or a pharmaceutically acceptable salt or solvate thereof.

In certain aspects, the present disclosure provides a method of treating a cancer comprising a Ras mutant (e.g., G12S, G12C, and/or G13C) protein, comprising inhibiting the Ras mutant protein of said subject by administering to said subject a compound, wherein the compound is characterized in that upon contacting the Ras mutant protein, the Ras mutant protein activity or function is inhibited (e.g., partially inhibited or completely inhibited), such that said inhibited Ras mutant protein exhibits reduced Ras signaling output (e.g., compared to a corresponding Ras protein not contacted by the compound).

In certain aspects, the present disclosure provides a method of modulating activity of a Ras protein (e.g., K-Ras, mutant K-Ras, G12S, G12C, and/or G13C), comprising contacting a Ras protein with an effective amount of a compound described herein, or a pharmaceutically acceptable salt or solvate thereof, thereby modulating the activity of the Ras protein.

In certain aspects, the present disclosure provides a method of inhibiting cell growth, comprising administering an effective amount of a compound described herein, or a pharmaceutically acceptable salt or solvate thereof, to a cell expressing a Ras (e.g., K-Ras) protein, thereby inhibiting growth of said cells. In some embodiments, the subject method comprises administering an additional agent to said cell.

In certain aspects, the present disclosure provides a method of treating a disease mediated at least in part by a Ras protein, such as K-Ras or a mutant thereof, in a subject in need thereof, comprising administering to the subject an effective amount of a compound disclosed herein, or a pharmaceutically acceptable salt or solvate thereof. In some embodiments, the disease is cancer, such as a solid tumor or a hematological cancer. In some embodiments, the method further comprises administering an additional agent to the subject, such as a SHP2 inhibitor, a SOS inhibitor, an EGFR inhibitor, a MEK inhibitor, an ERK inhibitor, a CDK4/6 inhibitor, a BRAF inhibitor, or a combination thereof.

In certain aspects, the present disclosure provides a method of inhibiting activity of a Ras protein, such as K-Ras or a mutant thereof, comprising contacting the Ras protein with a compound disclosed herein, or a pharmaceutically acceptable salt or solvate thereof. In some embodiments, the compound exhibits an IC50 against the Ras protein of less than 10 μM, such as less than 5 μM, 1 μM, 500 nM, 100 nM, 50 nM, 10 nM, 5 nM, 1 nM, 500 pM, 50 pM, 10 pM or less.

In certain aspects, the present disclosure provides a method of treating a Ras-mediated cancer in a subject in need thereof, comprising administering to the subject a SHP2 inhibitor, a SOS inhibitor, an EGFR inhibitor, a MEK inhibitor, an ERK inhibitor, a CDK4/6 inhibitor, or a BRAF inhibitor and an effective amount of a compound disclosed herein, such as a compound of Formula (II), or a pharmaceutically acceptable salt or solvate thereof.

In some embodiments, the cancer is a solid tumor. In some embodiments, the cancer is a hematological cancer.

In practicing any of the methods disclosed herein, the Ras target to which a subject compound binds, either covalently or reversibly, can be a Ras mutant (e.g., G12S, G12C, and/or G13C), including a mutant of K-Ras, H-Ras, or N-Ras. In some embodiments, the methods of treating cancer can be applied to treat a solid tumor or a hematological cancer. In some embodiments, the cancer being treated can be, without limitation, prostate cancer, brain cancer, colon cancer, rectal cancer, renal-cell carcinoma, liver cancer, various lung cancers including non-small cell carcinoma of the lung, cancer of the small intestine, cancer of the esophagus, melanoma, bone cancer, pancreatic cancer, skin cancer, cancer of the head or neck, cutaneous or intraocular malignant melanoma, uterine cancer, ovarian cancer, rectal cancer, cancer of the anal region, stomach cancer, testicular cancer, uterine cancer, carcinoma of the fallopian tubes, carcinoma of the endometrium, carcinoma of the cervix, carcinoma of the vagina, carcinoma of the vulva, Hodgkin's Disease, non-Hodgkin's lymphoma, cancer of the endocrine system, cancer of the thyroid gland, cancer of the parathyroid gland, cancer of the adrenal gland, sarcoma of soft tissue, cancer of the urethra, cancer of the penis, solid tumors of childhood, cancer of the bladder, cancer of the kidney or ureter, carcinoma of the renal pelvis, neoplasm of the central nervous system (CNS), primary CNS lymphoma, tumor angiogenesis, spinal axis tumor, brain stem glioma, pituitary adenoma, Kaposi's sarcoma, epidermoid cancer, squamous cell cancer, T-cell lymphoma, environmentally induced cancers, combinations of said cancers, and metastatic lesions of said cancers. In some embodiments is a method of treating cancer in a subject in need thereof, comprising administering to the subject a therapeutically effective amount of a compound described herein, or a pharmaceutically acceptable salt or solvate thereof, wherein the cancer is a hematological cancer. In some embodiments is a method of treating cancer in a subject in need thereof, comprising administering to the subject a therapeutically effective amount of a compound described herein, or a pharmaceutically acceptable salt or solvate thereof, wherein the cancer is a hematological cancer selected from one or more of chronic lymphocytic leukemia (CLL), acute leukemias, acute lymphoid leukemia (ALL), B-cell acute lymphoid leukemia (B-ALL), T-cell acute lymphoid leukemia (T-ALL), chronic myelogenous leukemia (CML), B cell prolymphocytic leukemia, blastic plasmacytoid dendritic cell neoplasm, Burkitt's lymphoma, diffuse large B cell lymphoma, follicular lymphoma, hairy cell leukemia, small cell- or a large cell-follicular lymphoma, malignant lymphoproliferative conditions, MALT lymphoma, mantle cell lymphoma, marginal zone lymphoma, multiple myeloma, myelodysplasia and myelodysplastic syndrome, non-Hodgkin's lymphoma, Hodgkin's lymphoma, plasmablastic lymphoma, plasmacytoid dendritic cell neoplasm, Waldenstrom macroglobulinemia, and pre-leukemia. In some embodiments is a method of treating cancer in a subject in need thereof, comprising administering to the subject a therapeutically effective amount of a compound described herein, or a pharmaceutically acceptable salt or solvate thereof, wherein the cancer is one or more cancers selected from the group consisting of chronic lymphocytic leukemia (CLL), acute myeloid leukemia (AML), T-cell acute lymphoblastic leukemia (T-ALL), B cell acute lymphoblastic leukemia (B-ALL), and/or acute lymphoblastic leukemia (ALL).

Any of the treatment methods disclosed herein can be administered alone or in combination or in conjunction with another therapy or another agent. By “combination” it is meant to include (a) formulating a subject composition containing a subject compound together with another agent, or (b) using the subject composition separate from the another agent as an overall treatment regimen. By “conjunction” it is meant that the another therapy or agent is administered either simultaneously, concurrently or sequentially with a subject composition comprising a compound disclosed herein, with no specific time limits, wherein such conjunctive administration provides a therapeutic effect.

In some embodiments, a subject treatment method is combined with surgery, cellular therapy, chemotherapy, radiation, and/or immunosuppressive agents. Additionally, compositions of the present disclosure can be combined with other therapeutic agents, such as other anti-cancer agents, anti-allergic agents, anti-nausea agents (or anti-emetics), pain relievers, cytoprotective agents, immunostimulants, and combinations thereof. In one embodiment, a subject treatment method is combined with a chemotherapeutic agent.

Exemplary chemotherapeutic agents include an anthracycline (e.g., doxorubicin (e.g., liposomal doxorubicin)), a vinca alkaloid (e.g., vinblastine, vincristine, vindesine, vinorelbine), an alkylating agent (e.g., cyclophosphamide, decarbazine, melphalan, ifosfamide, temozolomide), an immune cell antibody (e.g., alemtuzamab, gemtuzumab, rituximab, ofatumumab, tositumomab, brentuximab), an antimetabolite (including, e.g., folic acid antagonists, pyrimidine analogs, purine analogs and adenosine deaminase inhibitors (e.g., fludarabine)), a TNFR glucocorticoid induced TNFR related protein (GITR) agonist, a proteasome inhibitor (e.g., aclacinomycin A, gliotoxin or bortezomib), an immunomodulator such as thalidomide or a thalidomide derivative (e.g., lenalidomide). Additional chemotherapeutic agents contemplated for use in combination include busulfan (Myleran®), busulfan injection (Busulfex®), cladribine (Leustatin®), cyclophosphamide (Cytoxan® or Neosar®), cytarabine, cytosine arabinoside (Cytosar-U®), cytarabine liposome injection (DepoCyt®), daunorubicin hydrochloride (Cerubidine®), daunorubicin citrate liposome injection (DaunoXome®), dexamethasone, doxorubicin hydrochloride (Adriamycin®, Rubex®), etoposide (Vepesid®), fludarabine phosphate (Fludara®), hydroxyurea (Hydrea®), Idarubicin (Idamycin®), mitoxantrone (Novantrone®), Gemtuzumab Ozogamicin (Mylotarg®), anastrozole (Arimidex®), bicalutamide (Casodex®), bleomycin sulfate (Blenoxane®), busulfan injection (Busulfex®), capecitabine (Xeloda®), N4-pentoxycarbonyl-5-deoxy-5-fluorocytidine, carboplatin (Paraplatin®), carmustine (BiCNU®), chlorambucil (Leukeran®), cisplatin (Platinol®), dacarbazine (DTIC-Dome®), dactinomycin (Actinomycin D, Cosmegan), dexamethasone, docetaxel (Taxotere®), 5-fluorouracil (Adrucil®, Efudex®), flutamide (Eulexin®), tezacitibine, Gemcitabine (difluorodeoxycitidine), ifosfamide (IFEX®), irinotecan (Camptosar®), L-asparaginase (ELSPAR®), leucovorin calcium, melphalan (Alkeran®), 6-mercaptopurine (Purinethol®), methotrexate (Folex®), mitoxantrone (Novantrone®), mylotarg, paclitaxel (Taxol®), phoenix (Yttrium90/MX-DTPA), pentostatin, polifeprosan 20 with carmustine implant (Gliadel®), tamoxifen citrate (Nolvadex®), teniposide (Vumon®), 6-thioguanine, thiotepa, tirapazamine (Tirazone®), topotecan hydrochloride for injection (Hycamptin®), vinblastine (Velban®), vincristine (Oncovin®), and vinorelbine (Navelbine®).

Anti-cancer agents of particular interest for combinations with a compound of the present disclosure include: anthracyclines; alkylating agents; antimetabolites; drugs that inhibit either the calcium dependent phosphatase calcineurin or the p70S6 kinase FK506 or inhibit the p70S6 kinase; mTOR inhibitors; immunomodulators; anthracyclines; vinca alkaloids; proteosome inhibitors; GITR agonists; protein tyrosine phosphatase inhibitors; a CDK4 kinase inhibitor; a BTK inhibitor; a MKN kinase inhibitor; a DGK kinase inhibitor; or an oncolytic virus.

Exemplary antimetabolites include, without limitation, pyrimidine analogs, purine analogs and adenosine deaminase inhibitors: methotrexate (Rheumatrex®, Trexall®), 5-fluorouracil (Adrucil®, Efudex®, Fluoroplex®), floxuridine (FUDF®), cytarabine (Cytosar-U®, Tarabine PFS), 6-mercaptopurine (Puri-Nethol®)), 6-thioguanine (Thioguanine Tabloid®), fludarabine phosphate (Fludara®), pentostatin (Nipent®), pemetrexed (Alimta®), raltitrexed (Tomudex®), cladribine (Leustatin®), clofarabine (Clofarex®, Clolar®), azacitidine (Vidaza®), decitabine and gemcitabine (Gemzar®). Preferred antimetabolites include, cytarabine, clofarabine and fludarabine.

Exemplary alkylating agents include, without limitation, nitrogen mustards, ethylenimine derivatives, alkyl sulfonates, nitrosoureas and triazenes: uracil mustard (Aminouracil Mustard®, Chlorethaminacil®, Demethyldopan®, Desmethyldopan®, Haemanthamine®, Nordopan®, Uracil nitrogen Mustard®, Uracillost®, Uracilmostaza®, Uramustin®, Uramustine®), chlormethine (Mustargen®), cyclophosphamide (Cytoxan®, Neosar®, Clafen®, Endoxan®, Procytox®, Revimmune™), ifosfamide (Mitoxana®), melphalan (Alkeran®), Chlorambucil (Leukeran®), pipobroman (Amedel®, Vercyte®), triethylenemelamine (Hemel®, Hexalen®, Hexastat®), triethylenethiophosphoramine, Temozolomide (Temodar®), thiotepa (Thioplex®), busulfan (Busilvex®, Myleran®), carmustine (BiCNU®), lomustine (CeeNU®), streptozocin (Zanosar®), and Dacarbazine (DTIC-Dome®). Additional exemplary alkylating agents include, without limitation, Oxaliplatin (Eloxatin®); Temozolomide (Temodar® and Temodal®); Dactinomycin (also known as actinomycin-D, Cosmegen®); Melphalan (also known as L-PAM, L-sarcolysin, and phenylalanine mustard, Alkeran®); Altretamine (also known as hexamethylmelamine (HMM), Hexalen®); Carmustine (BiCNU®); Bendamustine (Treanda®); Busulfan (Busulfex® and Myleran®); Carboplatin (Paraplatin®); Lomustine (also known as CCNU, CeeNU®); Cisplatin (also known as CDDP, Platinol® and Platinol®-AQ); Chlorambucil (Leukeran®); Cyclophosphamide (Cytoxan® and Neosar®); Dacarbazine (also known as DTIC, DIC and imidazole carboxamide, DTIC-Dome®); Altretamine (also known as hexamethylmelamine (HMM), Hexalen®); Ifosfamide (Ifex®); Prednumustine; Procarbazine (Matulane®); Mechlorethamine (also known as nitrogen mustard, mustine and mechloroethamine hydrochloride, Mustargen®); Streptozocin (Zanosar®); Thiotepa (also known as thiophosphoamide, TESPA and TSPA, Thioplex®); Cyclophosphamide (Endoxan®, Cytoxan®, Neosar®, Procytox®, Revimmune®); and Bendamustine HCl (Treanda®).

In certain aspects, compositions provided herein can be administered in combination with radiotherapy, such as radiation. Whole body radiation may be administered at 12 Gy. A radiation dose may comprise a cumulative dose of 12 Gy to the whole body, including healthy tissues. A radiation dose may comprise from 5 Gy to 20 Gy. A radiation dose may be 5 Gy, 6 Gy, 7 Gy, 8 Gy, 9 Gy, 10 Gy, 11 Gy, 12, Gy, 13 Gy, 14 Gy, 15 Gy, 16 Gy, 17 Gy, 18 Gy, 19 Gy, or up to 20 Gy. Radiation may be whole body radiation or partial body radiation. In the case that radiation is whole body radiation it may be uniform or not uniform. For example, when radiation may not be uniform, narrower regions of a body such as the neck may receive a higher dose than broader regions such as the hips.

Where desirable, an immunosuppressive agent can be used in conjunction with a subject treatment method. Exemplary immunosuppressive agents include but are not limited to cyclosporin, azathioprine, methotrexate, mycophenolate, and FK506, antibodies, or other immunoablative agents such as CAMPATH, anti-CD3 antibodies (e.g., muromonab, otelixizumab) or other antibody therapies, cytoxin, fludarabine, cyclosporin, FK506, rapamycin, mycophenolic acid, steroids, FR901228, cytokines, and irradiation, peptide vaccine, and any combination thereof. In accordance with the presently disclosed subject matter, the above-described various methods can comprise administering at least one immunomodulatory agent. In certain embodiments, the at least one immunomodulatory agent is selected from the group consisting of immunostimulatory agents, checkpoint immune blockade agents (e.g., blockade agents or inhibitors of immune checkpoint genes, such as, for example, PD-1, PD-L¹, CTLA-4, IDO, TIM3, LAG3, TIGIT, BTLA, VISTA, ICOS, KIRs and CD39), radiation therapy agents, chemotherapy agents, and combinations thereof. In some embodiments, the immunostimulatory agents are selected from the group consisting of IL-12, an agonist costimulatory monoclonal antibody, and combinations thereof. In one embodiment, the immunostimulatory agent is IL-12. In some embodiments, the agonist costimulatory monoclonal antibody is selected from the group consisting of an anti-4-1BB antibody (e.g., urelumab, PF-05082566), an anti-OX40 antibody (pogalizumab, tavolixizumab, PF-04518600), an anti-ICOS antibody (BMS986226, MEDI-570, GSK3359609, JTX-2011), and combinations thereof. In one embodiment, the agonist costimulatory monoclonal antibody is an anti-4-1BB antibody. In some embodiments, the checkpoint immune blockade agents are selected from the group consisting of anti-PD-L¹ antibodies (atezolizumab, avelumab, durvalumab, BMS-936559), anti-CTLA-4 antibodies (e.g., tremelimumab, ipilimumab), anti-PD-1 antibodies (e.g., pembrolizumab, nivolumab, cemiplimab), anti-LAG3 antibodies (e.g., C9B7W, 410C9), anti-B7-H3 antibodies (e.g., DS-5573a), anti-TIM3 antibodies (e.g., F38-2E2), and combinations thereof. In one embodiment, the checkpoint immune blockade agent is an anti-PD-L¹ antibody. In some cases, a compound of the present disclosure can be administered to a subject in conjunction with (e.g., before, simultaneously or following) bone marrow transplantation, T cell ablative therapy using either chemotherapy agents such as, fludarabine, external-beam radiation therapy (XRT), cyclophosphamide, or antibodies such as OKT3 or CAMPATH. In some cases, expanded cells can be administered before or following surgery. Alternatively, compositions comprising a compound described herein can be administered with immunostimulants. Immunostimulants can be vaccines, colony stimulating agents, interferons, interleukins, viruses, antigens, co-stimulatory agents, immunogenicity agents, immunomodulators, or immunotherapeutic agents. An immunostimulant can be a cytokine such as an interleukin. One or more cytokines can be introduced with modified cells provided herein. Cytokines can be utilized to boost function of modified T lymphocytes (including adoptively transferred tumor-specific cytotoxic T lymphocytes) to expand within a tumor microenvironment. In some cases, IL-2 can be used to facilitate expansion of the modified cells described herein. Cytokines such as IL-15 can also be employed. Other relevant cytokines in the field of immunotherapy can also be utilized, such as IL-2, IL-7, IL-12, IL-15, IL-21, or any combination thereof. An interleukin can be IL-2, or aldesleukin. Aldesleukin can be administered in low dose or high dose. A high dose aldesleukin regimen can involve administering aldesleukin intravenously every 8 hours, as tolerated, for up to about 14 doses at about 0.037 mg/kg (600,000 IU/kg). An immunostimulant (e.g., aldesleukin) can be administered within 24 hours after a cellular administration. An immunostimulant (e.g., aldesleukin) can be administered in as an infusion over about 15 minutes about every 8 hours for up to about 4 days after a cellular infusion. An immunostimulant (e.g., aldesleukin) can be administered at a dose from about 100,000 IU/kg, 200,000 IU/kg, 300,000 IU/kg, 400,000 IU/kg, 500,000 IU/kg, 600,000 IU/kg, 700,000 IU/kg, 800,000 IU/kg, 900,000 IU/kg, or up to about 1,000,000 IU/kg. In some cases, aldesleukin can be administered at a dose from about 100,000 IU/kg to 300,000 IU/kg, from 300,000 IU/kg to 500,000 IU/kg, from 500,000 IU/kg to 700,000 IU/kg, from 700,000 IU/kg to about 1,000,000 IU/kg.

In some embodiments, a compound described herein, or a pharmaceutically acceptable salt or solvate thereof, is administered in combination or in conjunction with one or more pharmacologically active agents selected from (1) an inhibitor of MEK (e.g., MEK1, MEK2) or of mutants thereof (e.g., trametinib, cobimetinib, binimetinib, selumetinib, refametinib, AZD6244); (2) an inhibitor of epidermal growth factor receptor (EGFR) and/or of mutants thereof (e.g., afatinib, erlotinib, gefitinib, lapatinib, cetuximab panitumumab, osimertinib, olmutinib, EGF-816); (3) an immunotherapeutic agent (e.g., checkpoint immune blockade agents, as disclosed herein); (4) a taxane (e.g., paclitaxel, docetaxel); (5) an anti-metabolite (e.g. antifolates such as methotrexate, raltitrexed, pyrimidine analogues such as 5-fluorouracil (5-FU), ribonucleoside and deoxyribonucleoside analogues, capecitabine and gemcitabine, purine and adenosine analogues such as mercaptopurine, thioguanine, cladribine and pentostatin, cytarabine (ara C), fludarabine); (6) an inhibitor of FGFR1 and/or FGFR2 and/or FGFR3 and/or FGFR4 and/or of mutants thereof (e.g., nintedanib); (7) a mitotic kinase inhibitor (e.g., a CDK4/6 inhibitor, such as, for example, palbociclib, ribociclib, abemaciclib); (8) an anti-angiogenic drug (e.g., an anti-VEGF antibody, such as, for example, bevacizumab); (9) a topoisomerase inhibitor (e.g. epipodophyllotoxins such as for example etoposide and etopophos, teniposide, amsacrin, topotecan, irinotecan, mitoxantrone); (10) a platinum-containing compound (e.g. cisplatin, oxaliplatin, carboplatin); (11) an inhibitor of ALK and/or of mutants thereof (e.g. crizotinib, alectinib, entrectinib, brigatinib); (12) an inhibitor of c-MET and/or of mutants thereof (e.g., K252a, SU11274, PHA665752, PF2341066); (13) an inhibitor of BCR-ABL and/or of mutants thereof (e.g., imatinib, dasatinib, nilotinib); (14) an inhibitor of ErbB2 (Her2) and/or of mutants thereof (e.g., afatinib, lapatinib, trastuzumab, pertuzumab); (15) an inhibitor of AXL and/or of mutants thereof (e.g., R⁴²⁸, amuvatinib, XL-880); (16) an inhibitor of NTRK1 and/or of mutants thereof (e.g., Merestinib); (17) an inhibitor of RET and/or of mutants thereof (e.g., BLU-667, Lenvatinib); (18) an inhibitor of A-Raf and/or B-Raf and/or C-Raf and/or of mutants thereof (RAF-709, LY-3009120, sorafenib, vemurafenib, dabrafenib, encorafenib, regorafenib, GDC-879); (19) an inhibitor of ERK and/or of mutants thereof (e.g., ulixertinib, MK-8353, LTT462, AZD0364, SCH772984, BIX02189, LY3214996, ravoxertinib); (20) an MDM2 inhibitor (e.g., HDM-201, NVP-CGM097, RG-71 12, MK-8242, RG-7388, SAR405838, AMG-232, DS-3032, RG-7775, APG-115); (21) an inhibitor of mTOR (e.g., rapamycin, temsirolimus, everolimus, ridaforolimus); (22) an inhibitor of BET (e.g., I-BET 151, I-BET 762, OTX-015, TEN-010, CPI-203, CPI-0610, olionon, RVX-208, ABBC-744, LY294002, AZD5153, MT-1, MS645); (23) an inhibitor of IGF1/2 and/or of IGF1-R (e.g., xentuzumab, MEDI-573); (24) an inhibitor of CDK9 (e.g., DRB, flavopiridol, CR8, AZD 5438, purvalanol B, AT7519, dinaciclib, SNS-032); (25) an inhibitor of farnesyl transferase (e.g., tipifarnib); (26) an inhibitor of SHIP pathway including SHIP2 inhibitor, as well as SHIP1 inhibitors; (27) an inhibitor of SRC (e.g., dasatinib); (28) an inhibitor of JAK (e.g. tofacitinib); (29) a PARP inhibitor (e.g. Olaparib, Rucaparib, Niraparib, Talazoparib), (30) a BTK inhibitor (e.g. Ibrutinib, Acalabrutinib, Zanubrutinib), (31) a ROS1 inhibitor (e.g., entrectinib), (32) an inhibitor of Src, FLT3, HDAC, VEGFR, PDGFR, LCK, Bcr-Abl or AKT (33) an inhibitor of KRAS G12C mutant (e.g., including but not limited to AMG510, MRTX849, and any covalent inhibitors binding to the cysteine residue 12 of Kras, the structures of which are publicly known) (e.g., an inhibitor of Ras G12C as described in US20180334454, US20190144444, US20150239900, U.S. Pat. No. 10,246,424, US20180086753, WO2018143315, WO2018206539, WO20191107519, WO2019141250, WO2019150305, U.S. Pat. No. 9,862,701, US20170197945, US20180086753, U.S. Pat. No. 10,144,724, US20190055211, US20190092767, US20180127396, US20180273523, U.S. Pat. No. 10,280,172, US20180319775, US20180273515, US20180282307, US20180282308, WO2019051291, WO2019213526, WO2019213516, WO2019217691, WO2019241157, WO2019217307, WO2020047192, WO2017087528, WO2018218070, WO2018218069, WO2018218071, WO2020027083, WO2020027084, WO2019215203, WO2019155399, WO2020035031, WO2014160200, WO2018195349, WO2018112240, WO2019204442, WO2019204449, WO2019104505, WO2016179558, WO2016176338, or related patents and applications, each of which is incorporated by reference in its entirety), (34) an SHC inhibitor (e.g., PP2, AID371185), (35) a GAB inhibitor (e.g., GAB-0001), (36) a GRB inhibitor, (37) a PI-3 kinase inhibitor (e.g., Idelalisib, Copanlisib, Duvelisib, Alpelisib, Taselisib, Perifosine, Buparlisib, Umbralisib, NVP-BEZ235-AN), (38) a MARPK inhibitor, (39) a CDK4/6 inhibitor (e.g., palbociclib, ribociclib, abemaciclib), (40) a MAPK inhibitor (e.g., VX-745, VX-702, RO-4402257, SCIO-469, BIRB-796, SD-0006, PH-797804, AMG-548, LY2228820, SB-681323, GW-856553, RWJ67657, BCT-197), or (41) a SHP pathway inhibitor, such as a SHP2 inhibitor (e.g., RMC-4630, ERAS-601,

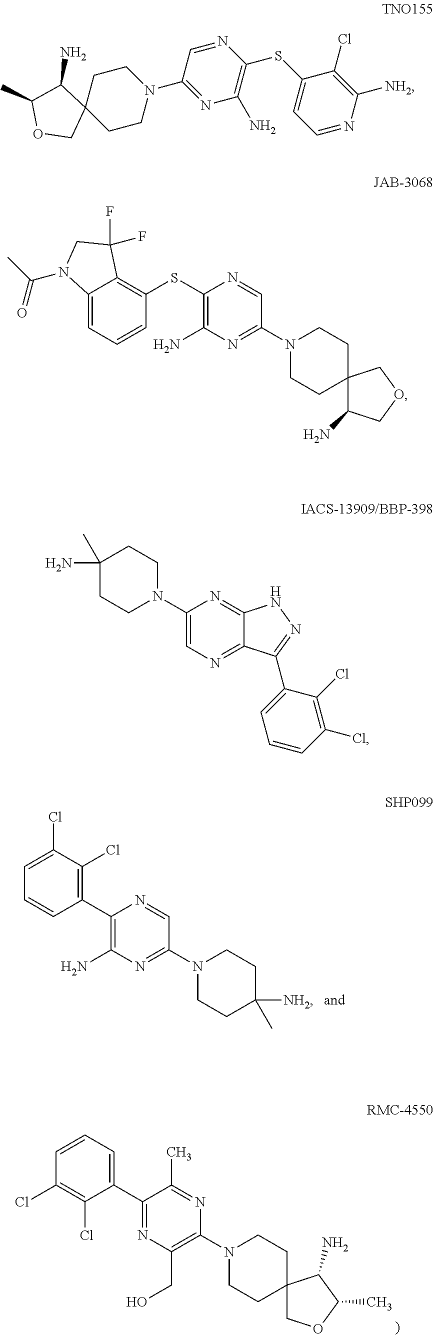 [SMILES: C[C@@H]1OCC2(CCN(c3cnc(Sc4ccnc(N)c4Cl)c(N)n3)CC2)[C@@H]1N ; CC(=O)N1CC(F)(F)c2c(Sc3ncc(N4CCC5(CC4)COC[C@H]5N)nc3N)cccc21 ; CC1(N)CCN(c2cnc3c(-c4cccc(Cl)c4Cl)n[nH]c3n2)CC1 ; CC1(N)CCN(c2cnc(-c3cccc(Cl)c3Cl)c(N)n2)CC1 ; Cc1nc(N2CCC3(CC2)CO[C@@H](C)[C@H]3N)c(CO)nc1-c1cccc(Cl)c1Cl]

or a SHP1 inhibitor. In some embodiments, a Ras inhibitor described herein, such as a compound, salt, or solvate of Formula (II), is administered in combination or in conjunction with one or more checkpoint immune blockade agents (e.g., anti-PD-1 and/or anti-PD-L¹ antibody, anti-CLTA-4 antibody). In some embodiments, a Ras inhibitor described herein, such as a compound, salt, or solvate of Formula (II), is administered in combination or in conjunction with one or more pharmacologically active agents comprising an inhibitor against one or more targets selected from: MEK, epidermal growth factor receptor (EGFR), FGFR1, FGFR2, FGFR3, mitotic kinase, topoisomerase, ALK, ALK5, c-MET, ErbB2, AXL, NTRK1, RET, A-Raf, B-Raf, C-Raf, ERK, MDM2, mTOR, BET, IGF1/2, IGF1-R, CDK9, SHIP1, SHIP2, SHP2, SRC, JAK, PARP, BTK, FLT3, HDAC, VEGFR, PDGFR, LCK, Bcr-Abl, AKT, KrasG12C mutant, and ROS1. In some embodiments, any of the compounds herein that is capable of binding a Ras protein (e.g., KRAS, mutant Ras protein) to modulate activity of such Ras mutant (e.g., G12C, G12S, or G13C) may be administered in combination or in conjunction with one or more additional pharmacologically active agents comprising an inhibitor of SOS (e.g., SOS1, SOS2) or of mutants thereof. In some embodiments, the additional pharmacologically active agent administered in combination or in conjunction with a compound described herein (e.g., compound capable of binding a Ras protein) is an inhibitor of SOS (e.g., SOS1, SOS2). In some embodiments, the additional pharmacologically active agent administered in combination or in conjunction with a compound (e.g., compound capable of binding a Ras protein) described herein is an inhibitor of SOS (e.g., SOS1, SOS2). In some embodiments, the additional pharmacologically active agent administered in combination or in conjunction with a compound (e.g., compound capable of binding a Ras protein) described herein is an inhibitor of SOS (e.g., SOS1, SOS2) selected from

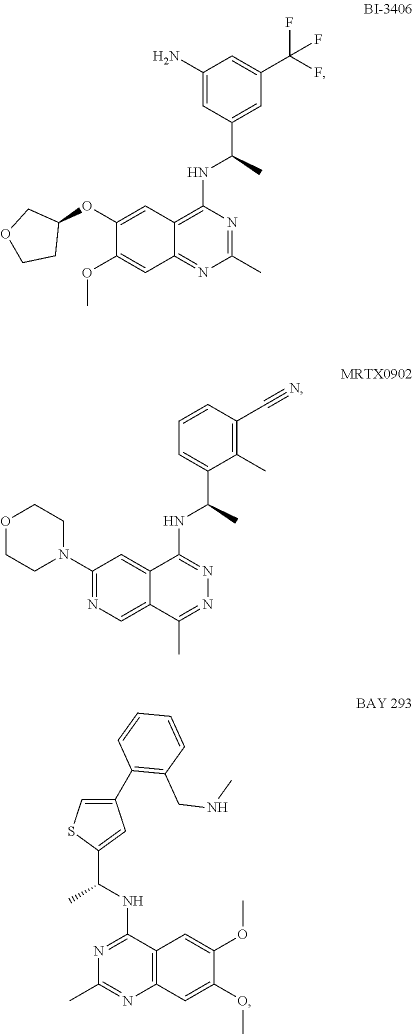

RMC-5845, and BI-1701963. In some embodiments, the additional pharmacologically active agent administered in combination or in conjunction with a compound described herein (e.g., compound capable of binding a Ras protein) is an inhibitor of SOS (e.g., SOS1, SOS2) described in WO2021092115, WO2018172250, WO2019201848, WO2019122129, WO2018115380, WO2021127429, WO2020180768, or WO2020180770, all of which are herein incorporated by reference in their entirety for all purposes.

In some embodiments, any of the compounds herein that is capable of binding a Ras protein (e.g., Kras) to modulate activity of such Ras protein may be administered in combination or in conjunction with one or more checkpoint immune blockade agents (e.g., anti-PD-1 and/or anti-PD-L¹ antibody, anti-CLTA-4 antibody).

In some embodiments, a compound described herein, such as a compound, salt, or solvate of Formula (II), is administered in combination or in conjunction with one or more pharmacologically active agents comprising an inhibitor of: (1) SOS1 or a mutant thereof (e.g., RMC-5845, BI-3406, BAY-293, MRTX0902, BI-1701963); (2) SHP2 or a mutant thereof (e.g., 6-(4-amino-4-methylpiperidin-1-yl)-3-(2,3-dichlorophenyl) pyrazin-2-amine, TNO155, RMC-4630, ERAS-601, JAB-3068, IACS-13909/BBP-398, SHP099, RMC-4550); (3) SHC or a mutant thereof (e.g., PP2, AID371185); (4) GAB or a mutant thereof (e.g., GAB-0001); (5) GRB or a mutant thereof; (6) JAK or a mutant thereof (e.g., tofacitinib); (7) A-RAF, B-RAF, C-RAF, or a mutant thereof (e.g., RAF-709, LY-3009120); (8) BRAF or a mutant thereof (e.g., sorafenib, vemurafenib, dabrafenib, encorafenib, regorafenib, GDC-879); (9) MEK or a mutant thereof (e.g., trametinib, cobimetinib, binimetinib, selumetinib, refametinib, AZD6244); (10) ERK or a mutant thereof (e.g., ulixertinib, MK-8353, LTT462, AZD0364, SCH772984, BIX02189, LY3214996, ravoxertinib); (11) PI3K or a mutant thereof (e.g., idelalisib, copanlisib, duvelisib, alpelisib, taselisib, perifosine, buparlisib, umbralisib, NVP-BEZ235-AN); (12) MAPK or a mutant thereof (e.g., VX-745, VX-702, RO-4402257, SCIO-469, BIRB-796, SD-0006, PH-797804, AMG-548, LY2228820, SB-681323, GW-856553, RWJ67657, BCT-197); (13) EGFR or a mutant thereof (e.g., afatinib, erlotinib, gefitinib, lapatinib, cetuximab panitumumab, osimertinib, olmutinib, EGF-816); (14) c-MET or a mutant thereof (e.g., K252a, SU11274, PHA665752, PF2341066); (15) ALK or a mutant thereof (e.g. crizotinib, alectinib, entrectinib, brigatinib); (16) FGFR1, FGFR-2, FGFR-3, FGFR-4 or a mutant thereof (e.g., nintedanib); (17) BCR-ABL or a mutant thereof (e.g., imatinib, dasatinib, nilotinib); (18) ErbB2 (Her2) or a mutant thereof (e.g., afatinib, lapatinib, trastuzumab, pertuzumab); (19) AXL or a mutant thereof (e.g., R⁴²⁸, amuvatinib, XL-880); (20) NTRK1 or a mutant thereof (e.g., merestinib); (21) ROS1 or a mutant thereof (e.g., entrectinib); (22) RET or a mutant thereof (e.g., BLU-667, Lenvatinib); (23) MDM2 or a mutant thereof (e.g., HDM-201, NVP-CGM097, RG-71 12, MK-8242, RG-7388, SAR405838, AMG-232, DS-3032, RG-7775, APG-115); (24) mTOR or a mutant thereof (e.g., rapamycin, temsirolimus, everolimus, ridaforolimus); (25) BET or a mutant thereof (e.g., I-BET 151, I-BET 762, OTX-015, TEN-010, CPI-203, CPI-0610, olionon, RVX-208, ABBC-744, LY294002, AZD5153, MT-1, MS645); (26) IGF1, IGF2, IGFIR, or a mutant thereof (e.g., xentuzumab, MEDI-573); (27) CDK9 or a mutant thereof (e.g., DRB, flavopiridol, CR8, AZD 5438, purvalanol B, AT7519, dinaciclib, SNS-032); or (28) CDK4/6 (e.g., palbociclib, ribociclib, abemaciclib).

In combination therapy, a compound provided herein and other anti-cancer agent(s) may be administered either simultaneously, concurrently or sequentially with no specific time limits, wherein such administration provides therapeutically effective levels of the two compounds in the body of the patient.

In some embodiments, the compound of the present disclosure and the other anti-cancer agent(s) are generally administered sequentially in any order by a suitable route, such as infusion or orally. The dosing regimen may vary depending upon the stage of the disease, physical fitness of the patient, safety profiles of the individual drugs, and tolerance of the individual drugs, as well as other criteria well-known to the attending physician and medical practitioner(s) administering the combination. The compound of the present disclosure and other anti-cancer agent(s) may be administered within minutes of each other, hours, days, or even weeks apart depending upon the particular cycle being used for treatment. In addition, the cycle could include administration of one drug more often than the other during the treatment cycle and at different doses per administration of the drug.

In some cases, a treatment regime may be dosed according to a body weight of a subject. In subjects who are determined obese (BMI>35) a practical weight may need to be utilized. BMI is calculated by: BMI=weight (kg)/[height (m)]².

Body weight may be calculated for men as 50 kg+2.3*(number of inches over 60 inches) or for women 45.5 kg+2.3 (number of inches over 60 inches). An adjusted body weight may be calculated for subjects who are more than 20% of their ideal body weight. An adjusted body weight may be the sum of an ideal body weight+(0.4×(Actual body weight−ideal body weight)). In some cases, a body surface area may be utilized to calculate a dosage. A body surface area (BSA) may be calculated by: BSA (m2)=√Height (cm)*Weight (kg)/3600.

In an aspect is provided a method of modulating activity of a Ras (e.g., K-Ras) protein, comprising contacting a Ras protein with an effective amount of a compound described herein, or a pharmaceutically acceptable salt or solvate thereof, thereby modulating the activity of the Ras (e.g., K-Ras) protein. In some embodiments, the subject method comprises administering an additional agent or therapy.

In some embodiments is a method of modulating activity of a Ras protein, comprising contacting a Ras protein with an effective amount of a compound described, or a pharmaceutically acceptable salt or solvate thereof, wherein said modulating comprises inhibiting the Ras (e.g., K-Ras) protein activity. In some embodiments is a method of modulating activity of a Ras protein, including Ras mutant (e.g., G12S, G12C, and/or G13C) proteins of K-Ras, H-Ras, and N-Ras, comprising contacting the Ras protein with an effective amount of a compound described herein, or a pharmaceutically acceptable salt or solvate thereof.

In some embodiments, provided is a method of reducing Ras signaling output in a cell by contacting the cell with a compound described herein. A reduction in Ras signaling can be evidenced by one or more members of the following: (i) an increase in steady state level of GDP-bound modified protein; (ii) a reduction in steady state level of GTP-bound Ras protein; (iii) a reduction of phosphorylated AKTs473, (iv) a reduction of phosphorylated ERKT202/y204, (v) a reduction of phosphorylated S6S235/236, (vi) a reduction of cell growth of a tumor cell expressing a Ras mutant (e.g., G12S, G12C, and/or G13C) protein, and (vii) a reduction in Ras interaction with a Ras-pathway signaling protein. Non-limiting examples of Ras-pathway signaling proteins include SOS (including SOS1 and SOS2), RAF, SHC, SHP (including SHP1 and SHP2), MEK, MAPK, ERK, GRB, RASA1, and GNAQ. In some embodiments, the reduction in Ras signaling output can be evidenced by two, three, four, five, six, or all of (i)-(vii) above. In some embodiments, the reduction of any one or more of (i)-(vii) can be 0.1-fold, 0.2-fold, 0.3-fold, 0.4-fold, 0.5-fold, 0.6-fold, 0.7-fold, 0.8-fold, 0.9-fold, 1-fold, 2-fold, 3-fold, 4-fold, 5-fold, 6-fold, 7-fold, 8-fold, 9-fold, 10-fold, 20-fold, 30-fold, 40-fold, 50-fold, 60-fold, 70-fold, 80-fold, 90-fold, 100-fold, 200-fold, 300-fold, 400-fold, 500-fold, 600-fold, 700-fold, 800-fold, 900-fold, 1000-fold, 2000-fold, 3000-fold, 4000-fold, 5000-fold, or more as compared to a control not treated with a subject compound. A reduction in cell growth can be demonstrated with the use of tumor cells or cell lines. A tumor cell line can be derived from a tumor in one or more tissues, e.g., pancreas, lung, ovary, biliary tract, intestine (e.g., small intestine, large intestine, colon), endometrium, stomach, hematopoietic tissue (e.g., lymphoid tissue), etc. Examples of the tumor cell line with a K-Ras mutation may include, but are not limited to, A549 (e.g., K-Ras G12S), AGS (e.g., K-Ras G12D), ASPC1 (e.g., K-Ras G12D), Calu-6 (e.g., K-Ras Q⁶¹K), CFPAC-1 (e.g., K-Ras G12V), CL40 (e.g., K-Ras G12D), COLO678 (e.g., K-Ras G12D), COR-L²³ (e.g., K-Ras G12V), DAN-G (e.g., K-Ras G12V), GP2D (e.g., K-Ras G12D), GSU (e.g., K-Ras G12F), HCT116 (e.g., K-Ras G13D), HECIA (e.g., K-Ras G12D), HEC1B (e.g., K-Ras G12F), HEC50B (e.g., K-Ras G12F), HEYA8 (e.g., K-Ras G12D or G13D), HPAC (e.g., K-Ras G12D), HPAFII (e.g., K-Ras G12D), HUCCT1 (e.g., K-Ras G12D), KARPAS620 (e.g., K-Ras G13D), KOPN8 (e.g., K-Ras G13D), KP-3 (e.g., K-Ras G12V), KP-4 (e.g., K-Ras G12D), L3.3 (e.g., K-Ras G12D), LoVo (e.g., K-Ras G13D), LS180 (e.g., K-Ras G12D), LS513 (e.g., K-Ras G12D), MCAS (e.g., K-Ras G12D), NB4 (e.g., K-Ras A18D), NCI-H1355 (e.g., K-Ras G13C), NCI-H1573 (e.g., K-Ras G12A), NCI-H1944 (e.g., K-Ras G13D), NCI-H2009 (e.g., K-Ras G12A), NCI-H441 (e.g., K-Ras G12V), NCI-H747 (e.g., K-Ras G13D), NOMO-1 (e.g., K-Ras G12D), OV7 (e.g., K-Ras G12D), PANC0203 (e.g., K-Ras G12D), PANC0403 (e.g., K-Ras G12D), PANC0504 (e.g., K-Ras G12D), PANC0813 (e.g., K-Ras G12D), PANC1 (e.g., K-Ras G12D), Panc-10.05 (e.g., K-Ras G12D), PaTu-8902 (e.g., K-Ras G12V), PK1 (e.g., K-Ras G12D), PK45H (e.g., K-Ras G12D), PK59 (e.g., K-Ras G12D), SK-CO-1 (e.g., K-Ras G12V), SKLUI (e.g., K-Ras G12D), SKM-1 (e.g., K-Ras K117N), SNUI (e.g., K-Ras G12D), SNU1033 (e.g., K-Ras G12D), SNU1197 (e.g., K-Ras G12D), SNU407 (e.g., K-Ras G12D), SNU410 (e.g., K-Ras G12D), SNU601 (e.g., K-Ras G12D), SNU61 (e.g., K-Ras G12D), SNU8 (e.g., K-Ras G12D), SNU869 (e.g., K-Ras G12D), SNU-C2A (e.g., K-Ras G12D), SU.86.86 (e.g., K-Ras G12D), SUIT2 (e.g., K-Ras G12D), SW1990 (e.g., K-Ras G12D), SW403 (e.g., K-Ras G12V), SW480 (e.g., K-Ras G12V), SW620 (e.g., K-Ras G12V), SW948 (e.g., K-Ras Q⁶¹L), T3M10 (e.g., K-Ras G12D), TCC-PAN2 (e.g., K-Ras G12R), TGBC11TKB (e.g., K-Ras G12D), and MIA Pa-Ca (e.g., MIA Pa-Ca 2 (e.g., K-Ras G12C)).

In an aspect is provided a modified Ras mutant protein comprising a compound described herein (or a remnant of a compound described herein wherein the remnant of said compound is modified from a stand alone compound described herein upon covalently bonding to an amino acid) covalently bonded to the amino acid corresponding to position 12 or 13 of SEQ ID No: 1. In some embodiments, such covalently bonded modified Ras mutant protein exhibits a reduced Ras signaling output (e.g., compared to a corresponding unmodified Ras mutant absent of the covalently bonded compound). In some embodiments, the modified Ras mutant protein comprises a compound described herein covalently bonded to the amino acid residue corresponding to position 12 or 13 of SEQ ID No: 1. In some embodiments, the modified Ras mutant protein comprises a compound described herein covalently bonded to the amino acid residue corresponding to position 12 or 13 of SEQ ID No: 1, wherein the Ras mutant protein is a human protein selected from KRas G12C, KRas G12S, KRas G13C, and KRas G13S. In some embodiments, the modified Ras mutant protein comprises a compound described herein covalently bonded to the amino acid residue corresponding to position 12 or 13 of SEQ ID No: 1, wherein the Ras mutant protein is a human KRAS mutant protein (e.g., G12S, G12C, G12D, G12V, G13C, and/or G13D). In some embodiments, the modified Ras mutant protein comprises a compound described herein covalently bonded to the amino acid residue corresponding to position 12 or 13 of SEQ ID No: 1, wherein the Ras mutant protein is a human KRas G12S protein. In some embodiments, the modified Ras mutant protein comprises a compound described herein covalently bonded to the amino acid residue corresponding to position 12 or 13 of SEQ ID No: 1, wherein the Ras mutant protein is a human KRas G12C protein. In some embodiments, the modified Ras mutant protein comprises a compound described herein covalently bonded to a protein of SEQ ID No. 4. In some embodiments, the modified Ras mutant protein comprises a compound described herein covalently bonded to the serine residue at position 12 of SEQ ID No. 4. In some embodiments, the modified Ras mutant protein comprises a compound described herein covalently bonded to the cysteine residue corresponding to position 12 of SEQ ID No. 1, wherein the wildtype glycine at position 12 is mutated to cysteine. In some embodiments, the modified Ras mutant protein comprises a compound described herein covalently bonded to the amino acid residue corresponding to position 12 or 13 of SEQ ID No: 1, wherein the Ras mutant protein is a mammalian Ras protein (including human protein) selected from NRas G12C, NRas G12S, NRas G13C, and NRas G13S. In some embodiments, the modified Ras mutant protein comprises a compound described herein covalently bonded to the amino acid residue corresponding to position 12 or 13 of SEQ ID No: 1, wherein the Ras mutant protein is a mammalian protein (including human protein) selected from HRas G12C, HRas G12S, HRas G13C, and HRas G13S. It will be understood that a compound described herein may be modified upon covalently binding an amino acid (e.g., mutant amino acid other than G) corresponding to position 12 or 13 of human KRas (e.g., SEQ ID. No: 1). A subject compound of the present disclosure encompasses a compound described herein immediately prior to covalently bonding the Ras mutant protein as well as the resulting compound covalently bonded to the modified Ras mutant protein. For example, a subject compound of the present disclosure can be covalently bonded to a mutant Ras protein to form a modified Ras mutant protein when a ring of the compound opened upon covalently bonding to the amino acid corresponding to position 12 or 13 of SEQ ID No: 1. The compound prior to and subsequent to such covalent binding are all considered a subject compound of the present disclosure.

In embodiments of a modified Ras mutant protein described herein, the reduced Ras signaling output is evidenced by one or more a reduced output selected from the group consisting of (i) an increase in steady state level of GDP-bound modified protein; (ii) a reduction of phosphorylated AKTs473, (iii) a reduction of phosphorylated ERK T202/Y204, (iv) a reduction of phosphorylated S6 S235/236, (v) reduction of cell growth of a tumor cell expressing a Ras mutant protein (e.g., G12C, G12S, G13C, or G13S), and (vi) reduction in Ras interaction with a Ras-pathway signaling protein.

In some embodiments, the modified Ras mutant protein described herein is formed by contacting a compound described herein with the serine residue of an unmodified Ras G12S mutant protein, wherein the compound comprises a moiety susceptible to reacting with a nucleophilic serine residue corresponding to position 12 of SEQ ID No: 4. In some embodiments, the compound comprises a staying group and a leaving group, and wherein said contacting results in release of the leaving group and formation of said modified protein. In some embodiments, the compound selectively labels the serine residue corresponding to position 12 of SEQ ID No. 4 (a G12S mutant) relative to a valine (G12V) residue or glycine residue (wildtype Kras) at the same position. In some embodiments, the compound selectively labels the serine residue as compared to (i) an aspartate residue of a K-Ras G12D mutant protein, said aspartate corresponding to residue 12 of SEQ ID NO: 2, and/or (ii) a valine residue of a K-Ras G12V mutant protein, said valine corresponding to residue 12 of SEQ ID NO: 3. In some embodiments, the compound selectively labels the cysteine residue at position 12 (a K-Ras G12C mutant, in which glycine is replaced with cysteine) relative to a valine (K-Ras G12V) residue or glycine (wildtype K-Ras) residue at the same position. In some embodiments, the compound selectively labels the cysteine or serine residues of a K-Ras mutant (i.e., K-Ras G12C or K-Ras G12S) as compared to (i) an aspartate residue of a K-Ras G12D mutant protein, said aspartate corresponding to residue 12 of SEQ ID NO: 2, and/or (ii) a valine residue of a K-Ras G12V mutant protein, said valine corresponding to residue 12 of SEQ ID NO: 3, by at least 1, 2, 3, 4, 5, or 10 fold or more, when assayed under comparable conditions.

In embodiments of the modified Ras mutant protein described herein, the compound covalently binds to the serine residue of an unmodified Ras G12S protein corresponding to position 12 of SEQ ID No: 4 in vitro. In embodiments of the modified Ras mutant protein described herein, the compound covalently binds to the serine residue of an unmodified K-Ras G12S protein corresponding to position 12 of SEQ ID No: 4 in vivo. In embodiments of the modified Ras mutant protein described herein, the compound covalently binds to the cysteine residue of an unmodified Ras G12C protein corresponding to position 12 a K-Ras G12C mutant, (in which glycine residue is replaced with cysteine) in vitro or in vivo. In embodiments of the modified Ras mutant protein described herein, the compound covalently binds to both the serine residue and the cysteine residue of an unmodified K-Ras G12S and K-Ras G12C protein, respectively, at position 12 of the respective protein in vitro or in vivo.

In an aspect is provided a method of treating cancer in a subject comprising a Ras mutant protein (e.g., KRas G12C, KRas G12S, KRas G13C, KRas G13S, NRas G12C, NRas G12S, NRas G13C, NRas G13S, HRas G12C, HRas G12S, HRas G13C, or HRas G13S), the method comprising modifying the Ras mutant protein of said subject by administering to said subject a compound described herein, wherein the compound is characterized in that upon contacting a Ras mutant protein, said Ras mutant protein is modified covalently at a residue corresponding to residue 12 or 13 of SEQ ID No: 1, such that said modified Ras mutant protein exhibits reduced Ras signaling output (e.g., compared to a control, such as an unmodified Ras mutant protein not covalently bonded with any compound such as a compound disclosed herein).

In some aspects, a subject compound exhibits one or more of the following characteristics: it is capable of reacting with a mutant residue (e.g., KRas G12C, KRas G12S, KRas G13C, KRas G13S, NRas G12C, NRas G12S, NRas G13C, NRas G13S, HRas G12C, HRas G12S, HRas G13C, or HRas G13S) of a Ras mutant protein and covalently modifying such Ras mutant and/or it comprises a moiety susceptible to reacting with a nucleophilic amino acid residue corresponding to position 12 or 13 of SEQ ID No: 1 (e.g., KRas G12C, KRas G12S, KRas G13C, KRas G13S, NRas G12C, NRas G12S, NRas G13C, NRas G13S, HRas G12C, HRas G12S, HRas G13C, or HRas G13S). In some embodiments, a subject compound, when used to modify a Ras mutant protein, reduces the signaling output of the Ras protein. In some embodiments, a subject compound exhibits an IC50 (against a mutant Ras (e.g., KRas G12C, KRas G12S, KRas G13C, KRas G13S, NRas G12C, NRas G12S, NRas G13C, NRas G13S, HRas G12C, HRas G12S, HRas G13C, or HRas G13S), as ascertained by reduction of Ras::SOS1 interaction) of less than 10 uM, such as less than 5 pM, 1 pM, 500 nM, 100 nM, 50 nM, 10 nM, 5 nM, 1 nM, 500 pM, 50 pM, 10 pM or less.

In some embodiments, a modified Ras mutant protein disclosed herein exhibits a reduced Ras signaling output. A reduction of signaling output can be ascertained by a wide variety of methods known in the art. For example, phosphorylation of a substrate or a specific amino acid residue thereof can be detected and/or quantified using one or more techniques, such as kinase activity assays, phospho-specific antibodies, Western blot, enzyme-linked immunosorbent assays (ELISA), cell-based ELISA, intracellular flow cytometry, mass spectrometry, and multi-analyte profiling. A host of readout can evidence a reduction of Ras signaling output, including without limitation: (i) an increase in steady state level of GDP-bound modified protein; (ii) a reduction of phosphorylated AKTs473, (iii) a reduction of phosphorylated ERK T202/Y204, (iv) a reduction of phosphorylated S6 S235/236, (v) reduction of cell growth of a tumor cell expressing a Ras mutant protein (e.g., KRas G12C, KRas G12S, KRas G13C, KRas G13S, NRas G12C, NRas G12S, NRas G13C, NRas G13S, HRas G12C, HRas G12S, HRas G13C, or HRas G13S), and (vi) reduction in Ras interaction with a Ras-pathway signaling protein. In some embodiments, a reduction is evidenced by 2, 3, 4 or more of items (i)-(vi). In some embodiments, the reduction in Ras signaling output can be evidenced by any one of (i)-(vi) as compared to control unmodified corresponding Ras protein that is not covalently bonded to any compound disclosed herein. For example, a control Ras protein, as described herein, can be a Ras protein (e.g., wildtype or mutated) that is not complexed with any subject compound of the present disclosure. The increase in item (i) or reduction in items (ii) through (vi) can be at least about 0.1-fold, 0.2-fold, 0.3-fold, 0.4-fold, 0.5-fold, 0.6-fold, 0.7-fold, 0.8-fold, 0.9-fold, 1-fold, 2-fold, 3-fold, 4-fold, 5-fold, 6-fold, 7-fold, 8-fold, 9-fold, 10-fold, 20-fold, 30-fold, 40-fold, 50-fold, 60-fold, 70-fold, 80-fold, 90-fold, 100-fold, 200-fold, 300-fold, 400-fold, 500-fold, 600-fold, 700-fold, 800-fold, 900-fold, 1000-fold, 2000-fold, 3000-fold, 4000-fold, 5000-fold, or more as compared to the control Ras protein. In some embodiments, a reduction in Ras interaction with a Ras-pathway signaling protein is established by a reduced interaction with SOS (including SOS1 and SOS2), RAF, SHC, SHP (including SHP1 and SHP2), MEK, MAPK, ERK, GRB, RASA1, or GNAQ.

Signaling output measured in terms of IC50 values can be obtained and a ratio of IC50 against one mutant relative to another mutant can be calculated. For instance, a selective reduction of K-Ras G12S signaling output can be evidenced by a ratio greater than one. In particular, a selective reduction of K-Ras G12S signaling relative to K-Ras G12D signaling or wildtype K-Ras signaling is evidenced if the ratio of IC50 (against K-Ras G12D or wildtype) to IC50 (against K-Ras G12S) is greater than 1.

It will be understood that when a compound described herein selectively labels the serine and/or cysteine residue of a K-Ras G12S or K-Ras G12C protein compared to another K-Ras protein(s) (e.g., WT, G12D, or G12V), the compound labels the K-Ras G12S or K-Ras G12C protein with greater speed or to a greater degree or by any other quantifiable measurement compared to the other K-Ras protein (e.g., WT, G12D, G12V), under similar or identical reaction conditions for the proteins being compared. In some embodiments, the greater labeling of K-Ras G12S and/or K-Ras G12C can be 0.1-fold, 0.2-fold, 0.3-fold, 0.4-fold, 0.5-fold, 0.6-fold, 0.7-fold, 0.8-fold, 0.9-fold, 1-fold, 2-fold, 3-fold, 4-fold, 5-fold, 6-fold, 7-fold, 8-fold, 9-fold, 10-fold, 20-fold, 30-fold, 40- fold, 50-fold, 60-fold, 70-fold, 80-fold, 90-fold, 100-fold, 200-fold, 300-fold, 400-fold, 500-fold, 600-fold, 700-fold, 800-fold, 900-fold, 1000-fold, 2000-fold, 3000-fold, 4000-fold, 5000-fold, or more as compared to another K-Ras protein (e.g., WT, G12D, or G12V).

In some embodiments, the compounds described herein, or a pharmaceutically acceptable salt or solvate thereof, are Ras modulators (including Ras inhibitors) capable of covalently modifying a Ras protein. Ras proteins being modified can be Ras G12S mutants or G12C mutants from K-Ras, H-Ras or N-Ras. The compounds disclosed herein, or pharmaceutically acceptable salts or solvates thereof, have a wide range of applications in therapeutics, diagnostics, and other biomedical research.

In an aspect is provided a method of treating cancer in a subject comprising a Ras G12S mutant protein, comprising modifying the Ras G12S mutant protein of said subject by administering to said subject a compound described herein, wherein said compound is characterized in that upon contacting the Ras G12S mutant protein, the Ras G12S mutant protein is modified covalently at a serine residue corresponding to residue 12 of SEQ ID No: 4, such that said modified K-Ras G12S protein exhibits reduced Ras signaling output (e.g., compared to a corresponding unmodified Ras protein unbound to the covalent compound).

In an aspect is provided a method of treating cancer in a subject comprising a Ras G12C mutant protein, comprising modifying the Ras G12C mutant protein of said subject by administering to said subject a compound described herein, wherein said compound is characterized in that upon contacting the Ras G12C mutant protein, the Ras G12C mutant protein is modified covalently at the cysteine residue corresponding to residue 12 of SEQ ID No: 1 (in which glycine at position 12 is replaced with cysteine), such that said modified K-Ras G12C protein exhibits reduced Ras signaling output (e.g., compared to a corresponding unmodified Ras protein unbound to the covalent compound).

In an aspect is provided a method of modulating activity of a Ras protein (e.g., K-Ras, mutant K-Ras, K-Ras G12S), comprising contacting a Ras protein with an effective amount of a compound described herein, or a pharmaceutically acceptable salt or solvate thereof, thereby modulating the activity of the Ras protein.

In practicing any of the methods disclosed herein, the Ras target to which a subject compound binds covalently can be a Ras mutant (e.g., KRas G12C, KRas G12S, KRas G13C, KRas G13S, NRas G12C, NRas G12S, NRas G13C, NRas G13S, HRas G12C, HRas G12S, HRas G13C, or HRas G13S).

Pharmaceutical compositions and methods of administration

In an aspect is provided a pharmaceutical composition comprising a compound described herein, or a pharmaceutically acceptable salt or solvate thereof, and a pharmaceutically acceptable excipient.

In some embodiments, a compound described herein, or a pharmaceutically acceptable salt or solvate thereof, is administered to a subject in a biologically compatible form suitable for administration to treat or prevent diseases, disorders, or conditions. Administration of a compound described herein can be in any pharmacological form including a therapeutically effective amount of a compound described herein, or a pharmaceutically acceptable salt or solvate thereof, alone or in combination with a pharmaceutically acceptable carrier.

In some embodiments, a compound described herein is administered as a pure chemical. In some embodiments, the compound described herein is combined with a pharmaceutically suitable or acceptable carrier (also referred to herein as a pharmaceutically suitable (or acceptable) excipient, physiologically suitable (or acceptable) excipient, or physiologically suitable (or acceptable) carrier) selected on the basis of a chosen route of administration and standard pharmaceutical practice as described, for example, in Remington: The Science and Practice of Pharmacy (Gennaro, 21^{st} Ed. Mack Pub. Co., Easton, PA (2005)).

Accordingly, provided herein is a pharmaceutical composition comprising at least one compound described herein, or a pharmaceutically acceptable salt, together with one or more pharmaceutically acceptable excipients. The excipient(s) (or carrier(s)) is acceptable or suitable if the excipient is compatible with the other ingredients of the composition and not deleterious to the recipient (i.e., the subject) of the composition.

In some embodiments of the methods described herein, a compound described herein is administered either alone or in combination with pharmaceutically acceptable carriers, excipients or diluents, in a pharmaceutical composition. Administration of a compound or composition described herein can be affected by any method that enables delivery of the compound to the site of action. These methods include, though are not limited to delivery via enteral routes (including oral, gastric or duodenal feeding tube, rectal suppository and rectal enema), parenteral routes (injection or infusion, including intraarterial, intracardiac, intradermal, intraduodenal, intramedullary, intramuscular, intraosseous, intraperitoneal, intrathecal, intravascular, intravenous, intravitreal, epidural and subcutaneous), inhalational, transdermal, transmucosal, sublingual, buccal and topical (including epicutaneous, dermal, enema, eye drops, ear drops, intranasal, vaginal) administration, although the most suitable route may depend upon for example the condition and disorder of the recipient. By way of example only, a compound described herein can be administered locally to the area in need of treatment, by, for example, local infusion during surgery, topical application such as creams or ointments, injection, catheter, or implant. The administration can also be by direct injection at the site of a diseased tissue or organ. In some embodiments, a compound described herein, or a pharmaceutically acceptable salt or solvate thereof, is administered orally.

In some embodiments of the methods described herein, a pharmaceutical composition suitable for oral administration is presented as a discrete unit such as a capsule, cachet or tablet, each containing a predetermined amount of the active ingredient; as a powder or granules; as a solution or a suspension in an aqueous liquid or a non-aqueous liquid; or as an oil-in-water liquid emulsion or a water-in-oil liquid emulsion. In some embodiments, the active ingredient is presented as a bolus, electuary, or paste.

Pharmaceutical compositions which can be used orally include tablets, push-fit capsules made of gelatin, as well as soft, sealed capsules made of gelatin and a plasticizer, such as glycerol or sorbitol. Tablets may be made by compression or molding, optionally with one or more accessory ingredients. Compressed tablets may be prepared by compressing in a suitable machine the active ingredient in a free-flowing form such as a powder or granules, optionally mixed with binders, inert diluents, or lubricating, surface active or dispersing agents. Molded tablets may be made by molding in a suitable machine a mixture of the powdered compound moistened with an inert liquid diluent. In some embodiments, the tablets are coated or scored and are formulated so as to provide slow or controlled release of the active ingredient therein. All formulations for oral administration should be in dosages suitable for such administration. The push-fit capsules can contain the active ingredients in admixture with filler such as lactose, binders such as starches, and/or lubricants such as talc or magnesium stearate and, optionally, stabilizers. In soft capsules, the active compounds may be dissolved or suspended in suitable liquids, such as fatty oils, liquid paraffin, or liquid polyethylene glycols. In some embodiments, stabilizers are added. Dragee cores are provided with suitable coatings. For this purpose, concentrated sugar solutions may be used, which may optionally contain gum arabic, talc, polyvinyl pyrrolidone, carbopol gel, polyethylene glycol, and/or titanium dioxide, lacquer solutions, and suitable organic solvents or solvent mixtures. Dyestuffs or pigments may be added to the tablets or Dragee coatings for identification or to characterize different combinations of active compound doses.

In some embodiments of the methods described herein, pharmaceutical compositions are formulated for parenteral administration by injection, e.g., by bolus injection or continuous infusion. Formulations for injection may be presented in unit dosage form, e.g., in ampoules or in multi-dose containers, with an added preservative. The compositions may take such forms as suspensions, solutions or emulsions in oily or aqueous vehicles, and may contain formulatory agents such as suspending, stabilizing and/or dispersing agents. The compositions may be presented in unit-dose or multi-dose containers, for example sealed ampoules and vials, and may be stored in powder form or in a freeze-dried (lyophilized) condition requiring only the addition of the sterile liquid carrier, for example, saline or sterile pyrogen-free water, immediately prior to use. Extemporaneous injection solutions and suspensions may be prepared from sterile powders, granules and tablets of the kind previously described.

Pharmaceutical compositions for parenteral administration include aqueous and non-aqueous (oily) sterile injection solutions of the active compound which may contain antioxidants, buffers, bacteriostats and solutes which render the formulation isotonic with the blood of the intended recipient; and aqueous and non-aqueous sterile suspensions which may include suspending agents and thickening agents. Suitable lipophilic solvents or vehicles include fatty oils such as sesame oil, or synthetic fatty acid esters, such as ethyl oleate or triglycerides, or liposomes. Aqueous injection suspensions may contain substances which increase the viscosity of the suspension, such as sodium carboxymethyl cellulose, sorbitol, or dextran. Optionally, the suspension may also contain suitable stabilizers or agents which increase the solubility of the compounds to allow for the preparation of highly concentrated solutions.

Pharmaceutical compositions may also be formulated as a depot preparation. Such long-acting formulations may be administered by implantation (for example subcutaneously or intramuscularly) or by intramuscular injection. Thus, for example, the compounds may be formulated with suitable polymeric or hydrophobic materials (for example, as an emulsion in an acceptable oil) or ion exchange resins, or as sparingly soluble derivatives, for example, as a sparingly soluble salt.

### EXAMPLES

The following examples are provided for illustrative purposes only and not to limit the scope of the claims provided herein. Unless noted otherwise, all materials, such as reagents, starting materials and solvents, were purchased from commercial suppliers, such as Sigma-Aldrich, VWR, and the like, and were used without further purification. Reactions were run under nitrogen atmosphere, unless noted otherwise. The progress of reactions was monitored by thin layer chromatography (TLC), analytical high performance liquid chromatography (anal. HPLC), and mass spectrometry, the details of which may be provided in specific examples.

Reactions were worked up as described specifically in each preparation; commonly, reaction mixtures were purified by extraction and other purification methods such as temperature- and solvent-dependent crystallization, and precipitation. In addition, reaction mixtures were routinely purified by preparative HPLC, for example, using Microsorb C18 or Microsorb BDS column packings and conventional eluents. Progress of reactions was typically monitored by liquid chromatography mass spectrometry (LCMS). Characterization of isomers was typically done by Nuclear Overhauser effect spectroscopy (NOE). Characterization of reaction products was routinely carried out by mass spectrometry and/or 1H-NMR spectroscopy. For NMR measurement, samples were dissolved in deuterated solvent (CD₃OD, CDCl₃, or DMSO-d₆).

### Example 1a: Synthesis of (4R)-2-amino-4-(6-chloro-4-(1-ethyl-2-(1H-1,2,4-triazole-1-carbonyl)-2,6-diazaspiro[3.4]octan-6-yl)-8-fluoro-2-(((2R,7aS)-2-fluorotetrahydro-1H-pyrrolizin-7a(5H)-yl) methoxy)quinazolin-7-yl)-7-fluorobenzo[b]thiophene-3-carbonitrile (123)

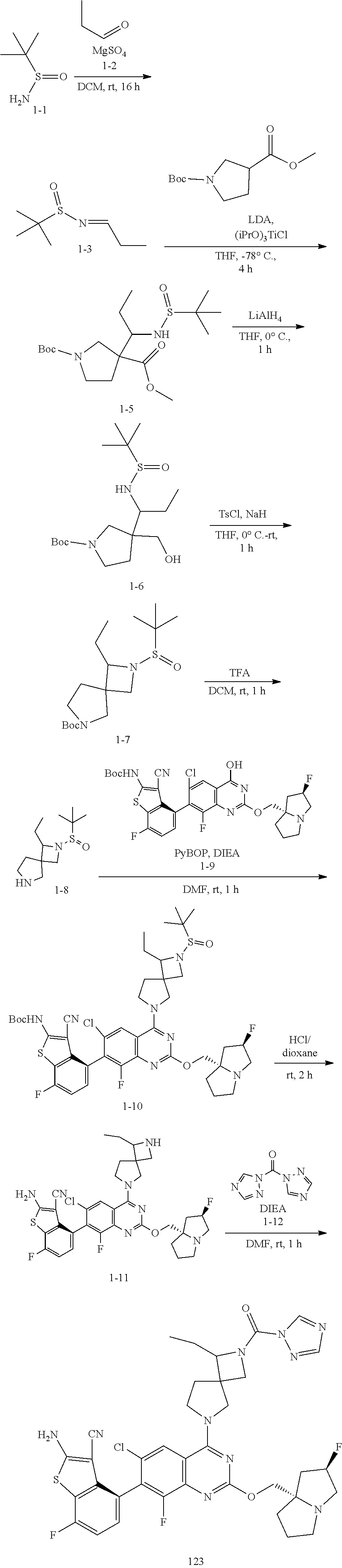

Step A: A mixture of 1-1 (5 g, 41.3 mmol), 1-2 (12 g, 206.6 mmol) and MgSO₄ (24.8 g, 206.6 mmol) in DCM (50 mL) was stirred at rt for 16 h. The reaction mixture was filtered and the filtrate was concentrated to dryness under reduced pressure. The residue was purified by silica gel column (PE/EA=5/1) to give 1-3 (5.4 g, yield: 81.8%) as a colorless oil. ESI-MS m/z: (M+H)⁺=161.0.

Step B: N-butyllithium (2.5 M in hexanes, 12.4 mL, 31.0 mmol) was added dropwise at 0° C. to a solution of diisopropylamine (3.14 g, 31.0 mmol) in dry THF (30 mL) and the resulting solution was stirred at 0° C. for 0.5 h. The reaction mixture was then cooled to −78° C. followed by dropwise addition of a THF solution (40 mL) of 1-4 (5.7 g, 24.8 mmol). After the resulting mixture was stirred at −78° C. for 1 h, a solution of (i-PrO); TiCl (37.2 mL, 37.2 mmol) was added dropwise and the reaction mixture was stirred for 1 h. A solution of 1-3 (2 g, 12.4 mmol) in THF (5 mL) was then added dropwise and the reaction mixture was stirred for an additional 1 h. The reaction mixture was diluted with NH₄Cl (aq) (50 mL) and extracted with EtOAc (50 mL×3). The combined organic layers were washed with brine (20 mL), dried over anhydrous sodium sulfate, filtered and the filtrate was concentrated to dryness under reduced pressure. The residue was purified by column on silica gel (PE/EA=3/1) to give 1-5 as colorless oil. (3 g, yield: 62.5%). ESI-MS m/z: (M+H)⁺=391.4.

Step C: To a solution of 1-5 (3 g, 7.69 mmol) in dry THF (30 mL) was added LiAlH₄ (15.3 mL, 15.3 mmol) at 0° C. The mixture was stirred at 25° C. for 1 h, then 10H₂O·NaSO₄ was added. The reaction mixture was filtered and the filtrate was dried over anhydrous sodium sulfate, filtered and the filtrate was concentrated to dryness under reduced pressure to give 1-6 (2.4 g, yield: 86.3%) as colorless oil. ESI-MS m/z: (M+H)⁺=363.6.

Step D: To a solution of 1-6 (1 g, 2.76 mmol) and TsCl (0.787 g, 4.14 mmol) in dry THF (10 mL) was added NaH (60% dispersion in mineral oil, 0.552 g, 13.8 mmol) at 0° C. After stirring at 25° C. for 3 h, the reaction mixture was diluted with NH₄Cl (aq) (20 mL) and extracted with EA (20 mL×3). The combined organic layers were washed with brine (20 mL), dried over anhydrous sodium sulfate, filtered and the filtrate was concentrated to dryness under reduced pressure. The residue was purified by column on silica gel (PE/EA=3/1) to give 1-7 as a colorless oil. (200 mg, yield: 21%). ESI-MS m/z: (M+H)⁺=345.2.

Step E: To a solution of 1-7 (150 mg, 0.43 mmol) in DCM (2 mL) was added TFA (0.4 mL) at 0° C. The reaction mixture was stirred at 25° C. for 2 h, then the solvent was removed under reduced pressure to afford 1-8 (150 mg, crude) as a yellow oil. The crude product was used directly for the next step without further purification. ESI-MS m/z: (M+H)⁺=245.1.

Step F: To a solution of 1-9 (300 mg, 0.45 mmol) and PyBOP (360 mg, 0.69 mmol) in DMF (2 mL) were added compound 1-8 (150 mg, 0.6 mmol) and DIEA (120 mg, 0.93 mmol). The reaction mixture was stirred at room temperature for 1 h, then poured into water (50 mL) and extracted with ethyl acetate (50 mL×3). The combined organic layers were washed with brine (50 mL), dried over anhydrous sodium sulfate, filtered and the filtrate was concentrated to dryness under reduced pressure. The residue was purified by prep-TLC (DCM/NH₃:MeOH=15:1) to give 1-10 (140 mg, yield: 29.6%) as a yellow solid. ESI-MS m/z:(M+H)⁺=871.7.

Step G: 1-10 (140 mg, 0.16 mmol) was added to HCl/dioxane (4M in 1,4-dioxane solution, 2 mL). The reaction mixture was stirred at 20° C. for 2 h, then the solvent was removed under reduced pressure to afford compound 1-11 (120 mg, crude) as a yellow solid. The crude product was used directly for the next step without further purification. ESI-MS m/z: (M+H)⁺=668.1.

Step H: To a solution of compound 1-11 (120 mg, 0.18 mmol) and DIEA (92.8 mg, 0.72 mmol) in DMF (1.5 mL) was added compound 1-12 (88.5 mg, 0.54 mmol). The reaction mixture was stirred at room temperature for 1 h, then concentrated and purified by prep-HPLC (FA) to give 123 (27.41 mg, yield: 19.7%) as a white solid. ESI-MS m/z: (M+H)⁺=763.2. ¹H NMR (400 MHZ, DMSO-d6) δ 9.14 (m, 1H), 8.25-8.10 (m, 4H), 7.25-7.14 (m, 2H), 5.29 (d, J=53.6 Hz, 1H), 4.41-4.10 (m, 3H), 4.02-3.99 (m, 6H), 3.35 (s, 2H), 3.12 (s, 1H), 3.04 (s, 1H), 2.35-2.13 (m, 3H), 2.50-2.49 (m, 3H), 1.90-1.71 (m, 4H), 1.10-1.01 (m, 3H).

### Example 1b: Synthesis of N-(1-(7-(2-amino-3-cyano-7-fluorobenzo[b]thiophen-4-yl)-6-chloro-8-fluoro-2-(((2R,7aS)-2-fluorotetrahydro-1H-pyrrolizin-7a(5H)-yl)methoxy)quinazolin-4-yl)azepan-4-yl)-N-(2-cyanoethyl)-1H-1,2,4-triazole-1-carboxamide (166)

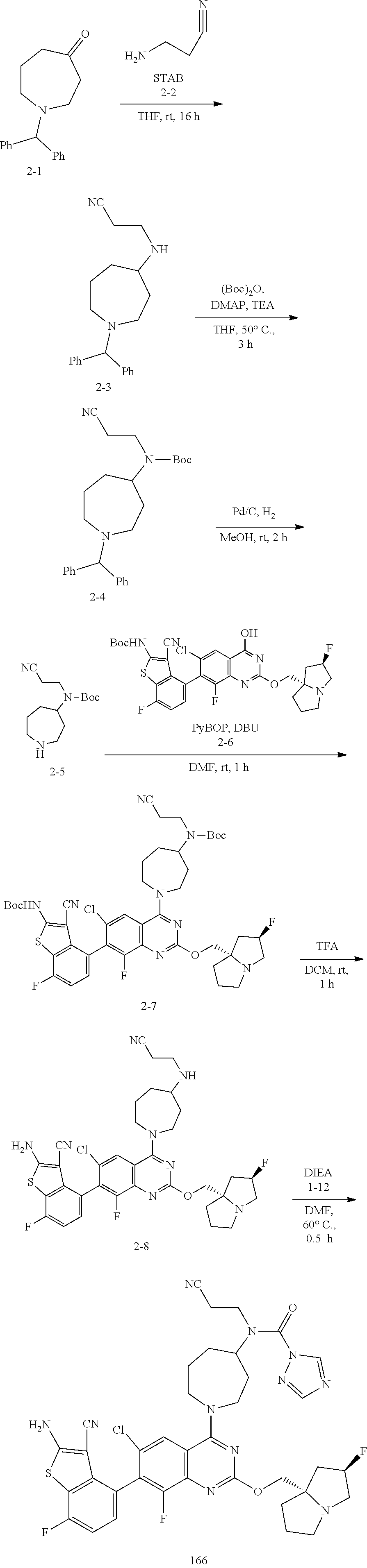

Step A: A mixture of 2-1 (2.0 g, 7.16 mmol) and 2-2 (500 mg, 7.16 mmol) in THF (20 mL) under N₂ was stirred at room temperature for 4 h, then NaBH (OAc) 3 (STAB) (2.13 g, 10.04 mol) was added at 0° C. under N₂. The mixture was stirred at rt overnight, then poured into ice water (100 mL) and extracted with EtOAc (300 mL). The organic layer was washed with brine (100 mL) and dried over anhydrous sodium sulfate. The filtrate was concentrated to dryness under reduced pressure. The residue was purified by silica gel column (PE/EA=1/2) to give 2-3 (1.2 g, yield: 50%) as a yellow oil. ESI-MS m/z: (M+H)⁺=334.2.

Step B: To a solution of 2-3 (1.2 g, 3.59 mmol) in THF (2 mL) was added DMAP (70.2 mg, 0.575 mmol) and DIEA (1.39 g, 10.78 mmol) under N₂, then (Boc)₂O (1.57 g, 7.18 mmol) was added at 0° C. The reaction mixture was stirred at 50° C. for 3 hours, then poured into water (100 mL) and the solution extracted with EtOAc (300 mL). The organic layer was washed with brine (100 mL), dried over anhydrous sodium sulfate, filtered and the filtrate concentrated to dryness under reduced pressure. The residue was purified by silica gel column (PE/EA=10/1) to give 2-4 (700 mg, yield: 45%) as a yellow solid. ESI-MS m/z: (M+H)⁺=434.5.

Step C: To a solution of 2-4 (700 mg, 1.62 mmol) in MeOH (10 mL) was added Pd/C (300 mg). The reaction mixture was stirred at rt under H₂ atmosphere for 2 h. The reaction mixture was diluted with methanol, filtered through diatomaceous earth and concentrated in vacuo to give 2-5 (500 mg, crude) as a colorless oily liquid.

Step D: To a solution of 2-6 (200 mg, 0.31 mmol), 2-5 (166 mg, 0.62 mmol) and PyBOP (484 mg, 0.93 mmol) in DMF (3 mL) was added DBU (116 mg, 0.93 mmol). The reaction mixture was stirred at 25° C. for 1 h, then poured into ice water (50 mL) and extracted with ethyl acetate (50 mL×3). The organic layer was washed with brine (50 mL), dried over anhydrous sodium sulfate, filtered and the filtrate concentrated to dryness under reduced pressure. The residue was purified by prep-TLC (DCM/MeOH=15/1) to give 2-7 (150 mg, yield: 54.2%) as a yellow solid. ESI-MS m/z: (M+H)⁺=895.3.

Step E: To a solution of 2-7 (150 mg, 0.17 mmol) in DCM (2 mL) was added TFA (1 mL). The reaction mixture was stirred at 20° C. for 1 h, then the solvent was removed under reduced pressure. The reaction mixture was diluted with a.q. NaHCO₃ (80 mL) and DCM:MeOH=10:1 (50×3 mL). The organic layer was washed with brine, dried over sodium sulfate, and filtered. The residue was purified by work up to give 2-8 (110 mg, yield: 94.8%) as a yellow solid. ESI-MS m/z: (M+H)⁺=695.2.

Step F: To a solution of 2-8 (40 mg, 0.06 mmol) and 1-12 (286 mg, 1.74 mmol) in DMF (1 mL) was added DIEA (225 mg, 1.74 mmol). The mixture was stirred at 60° C. for 0.5 h, then concentrated and purified by prep-HPLC (FA) to give 166 (10.20 mg, yield: 21.5%) as a white solid. ESI-MS m/z: (M+H)⁺=790.2. ¹H NMR (400 MHZ, DMSO-d6) δ 9.07 (d, J=2.0 Hz, 1H), 8.23 (d, J=17.6 Hz, 1H), 8.10 (s, 2H), 7.98 (d, J=4.4 Hz, 1H), 7.27-7.20 (m, 1H), 7.15 (t, J=8.8 Hz, 1H), 5.26 (d, J=55.6 Hz, 1H), 4.19-4.07 (m, 3H), 4.04-3.97 (m, 1H), 3.84-3.66 (m, 4H), 3.15-3.02 (m, 3H), 3.00 (s, 1H), 2.98-2.88 (m, 2H), 2.86-2.78 (m, 1H), 2.34-1.71 (m, 12H).

### Example 1c: Synthesis of (4R)-2-amino-4-(6-chloro-4-((1-(1-(3-chloro-1H-1,2,4-triazole-1-carbonyl) azetidin-2-yl)ethyl)(methyl)amino)-8-fluoro-2-(((2R,7aS)-2-fluorotetrahydro-1H-pyrrolizin-7a (5H)-yl) methoxy) quinazolin-7-yl)-7-fluorobenzo[b]thiophene-3-carbonitrile (172)

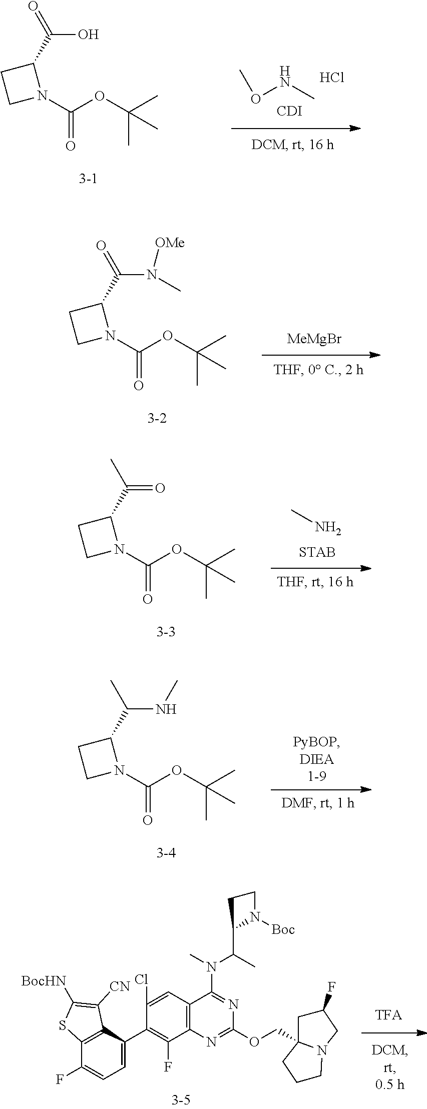

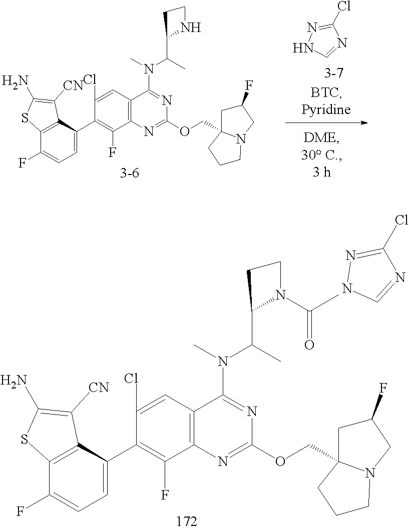

Step A: To a solution of 3-1 (5 g, 24.85 mmol) in dry DCM (100 mL) was added CDI (5.24 g, 32.32 mmol) and N,O-dimethylhydroxylamine hydrochloride (3.16 g, 32.40 mmol) at rt. The mixture was stirred at room temperature for 16 h, then diluted with DCM (100 mL) and washed with water (100 mL). The organic layer was separated and concentrated in vacuo, then the crude residue was purified by flash chromatography (PE:EA=2:1) to give 3-2 (5.4 g, yield: 89.0%) as colorless oily liquid.

Step B: To a solution of 3-2 (3.5 g, 14.336 mmol) in THF (35 mL) was added MeMgBr (23.90 mL, 24 mmol) at 0° C. The mixture was stirred at 0° C. for 2 h, then diluted with EA (100 mL) and washed with water (100 mL). The organic layer was separated, concentrated in vacuo, and the resulting residue purified by flash chromatography (PE:EA=2:1) to give 3-3 (2.5 g, yield: 87.7%) as colorless oily liquid.

Step C: To a solution of 3-3 (1 g, 5.0221 mmol) in THF (10 mL) was added methylamine tetrahydrofuran solution (2 M, 7.5 mL). The mixture was stirred at rt for 1 h under N₂, then STAB (2.129 g, 10.0453 mmol) was added. The mixture was stirred rt for 16 h under N₂, then diluted with a.q. NaHCO₃ (10 mL) and EA (100 mL). The organic layer was washed with brine (50 mL), dried over anhydrous sodium sulfate, filtered and the filtrate concentrated to dryness under reduced pressure. The residue was purified by flash chromatography (DCM:MeOH=20:1) to give 3-4 (650 mg, yield: 60.2%) as colorless oily liquid.

Step D: To a solution of 3-4 (75 mg, 0.3502 mmol) in DMF (1.5 mL) were added 1-9 (150 mg, 0.2325 mmol), PyBOP (242 mg, 0.4650 mmol) and DIEA (90 mg, 0.6963 mmol), and the resulting mixture was stirred under room temperature for 1 h. The mixture was diluted with EA (150 mL) and washed with brine (100 mL), then the organic layer was separated and concentrated in vacuo. The crude product was purified by flash chromatography (DCM:MeOH=15:1) to give 3-5 (160 mg, yield: 81.2%). ESI-MS m/z: (M+H)⁺=842.3.

Step E: To a solution of 3-5 (100 mg, 0.1189 mmol) in DCM (1 mL) was added TFA (1 mL). The reaction mixture was stirred at rt for 0.5 h, then solvent was removed under reduced pressure. The reaction mixture was diluted with a.q. NaHCO₃ (80 mL) and DCM:MeOH (10:1; 50 mL×3). The organic layer was washed with brine, dried over sodium sulfate, filtered, and concentrated to dryness. The residue was purified by work up to give 3-6 (100 mg, yield: 131.2%) as a purple solid. ESI-MS m/z: (M+H)⁺=642.2.

Step F: To a solution of 3-7 (137 mg, 1.33 mmol) and pyridine (0.43 mL, 5.32 mmol) in MeCN (4.5 mL) was added BTC (356 mg, 1.20 mmol) at 0° C. under N₂ atmosphere. The mixture was stirred at 0° C. for 2 h, then 1.5 mL of the mixture was added to a solution of 3-6 (80 mg, 0.1248 mmol) in DME (1 mL) and pyridine (0.43 mL, 5.32 mmol) at rt under N₂ atmosphere. The mixture was concentrated and purified by prep-HPLC (FA) to give 172 (4.31 mg, yield: 3.54%) as a white solid. ¹H NMR (400 MHZ, DMSO-d6) δ 9.25-8.55 (m, 1H), 8.22-8.02 (m, 2H), 7.88-7.62 (m, 1H), 7.33-7.09 (m, 2H), 5.37-3.81 (m, 7H), 3.19-2.96 (m, 6H), 2.86-2.77 (m, 1H), 2.43-1.64 (m, 8H), 1.52-1.24 (m, 3H).

### Example 1d: Synthesis of (R)-2-amino-4-(6-chloro-4-((1R,4R,7S)-2-(3-(difluoromethyl)-1H-1,2,4-triazole-1-carbonyl)-1-isopropyl-7-methyl-2,6-diazaspiro[3.5]nonan-6-yl)-8-fluoro-2-(((2R,7aS)-2-fluorotetrahydro-1H-pyrrolizin-7a(5H)-yl)methoxy)quinazolin-7-yl)-7-fluorobenzo[b]thiophene-3-carbonitrile (109)

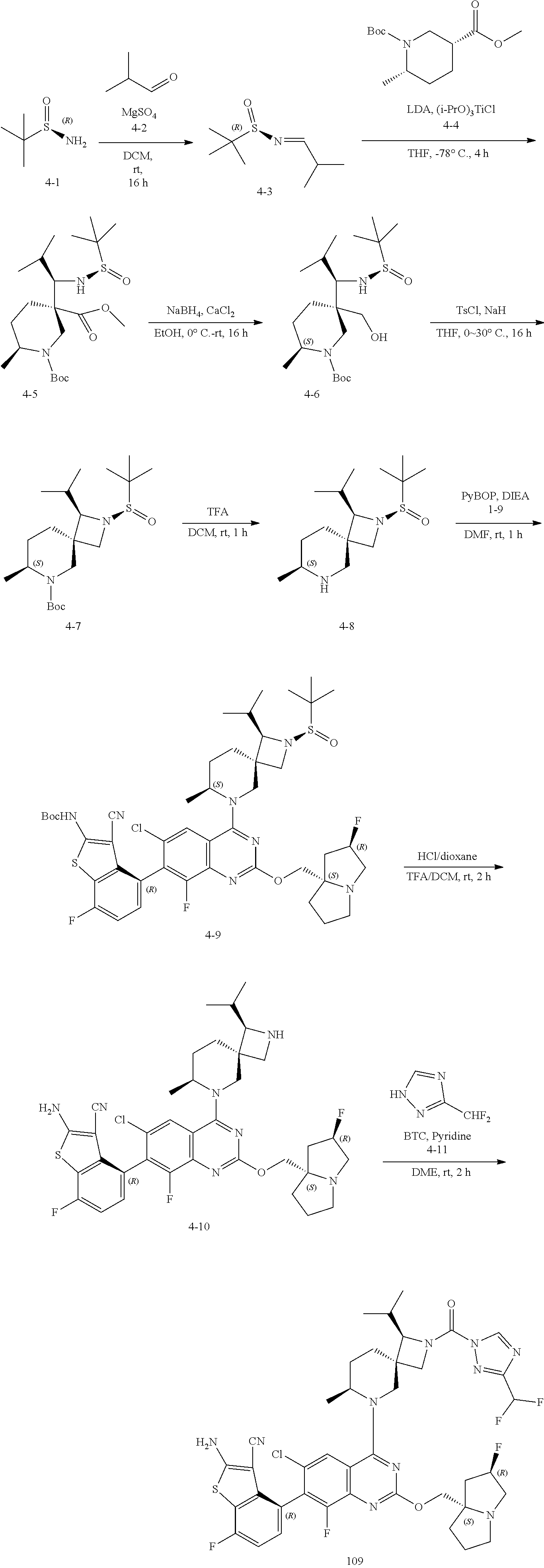

Step A: A mixture of 4-1 (5 g, 41.25 mmol), 4-2 (14.87 g, 206 mmol) and MgSO₄ (24.8 g, 206 mmol) in DCM (50 mL) was stirred at rt for 16 h. The reaction solution was filtered and the organic fraction was concentrated to dryness under reduced pressure. The residue was purified by silica gel chromatography (PE/EA=5/1) to give 4-3 (5 g, yield: 69.3%) as a colorless oil. ESI-MS m/z: (M+H)⁺=176.0.

Step B: To a solution of diisopropylamine (1.6 mL, 11.4 mmol) in THF (20 mL) was added n-BuLi (4.5 mL, 11.4 mmol, 2.5 M) at −20° C. under N₂. The reaction mixture was stirred at −20° C. for 45 min, then 4-4 (2.2 g, 8.57 mmol) in THF (10 mL) at −78° C. was added. The reaction mixture was stirred at −78° C. for 1 h, then a solution of (i-PrO)₃TiCl (14.3 mL, 14.3 mmol, 1M) at −78° C. was added and the resulting mixture stirred at −78° C. for 1 h. To the resulting mixture was added 4-3 (1.0 g, 5.7 mmol) in THF (5 mL) at −78° C. The reaction mixture was stirred at −78° C. for 1 h, then was diluted with a.q NH₄Cl (50 mL) and EA (50 mL) and filtered with diatomite. The organic layer was washed with brine, dried over sodium sulfate, filtered, and concentrated to dryness under reduced pressure to give a crude residue, which was purified by silica gel chromatography (PE:EA=3:1) to give 4-5 (1 g, yield: 41%) as a white solid. ESI-MS m/z: (M+H)⁺=433.4.

Step C: To a solution of 4-5 (1 g, 2.31 mmol) in dry EtOH (10 mL) was added CaCl₂) (1.02 g, 9.24 mmol) and NaBH₄ (0.697 g, 18.48 mmol) at 0° C. The mixture was stirred at 25° C. for 16 h before diluting with a.q citric acid (10 mL), water (40 mL) and EA (50 mL). The organic layer was washed with brine, dried over sodium sulfate, filtered, and concentrated to dryness under reduced pressure to give a crude residue, which was purified by silica gel chromatography (PE:EA=2:1) to give 4-6 (0.6 g, yield: 64%) as a white solid. ESI-MS m/z: (M+H)⁺=405.4.

Step D: To a solution of 4-6 (1 g, 2.47 mmol) and TsCl (706 mg, 3.71 mmol) in dry THF (30 mL) was added NaH (395 mg, 9.88 mmol, 60%) at 0° C. The mixture was stirred at 30° C. for 16 h, then diluted with NH₄Cl (aq) (30 mL) and extracted with EA (30 mL×3). The organic layer was washed with brine (20 mL), dried over anhydrous sodium sulfate, filtered and the filtrate concentrated to dryness under reduced pressure. The residue was purified by silica gel chromatography (PE/EA=3/1) to give 4-7 as a white solid. (700 mg, yield: 73%). ESI-MS m/z: (M+H)⁺=387.3.

Step E: To a solution of 4-7 (300 mg, 0.78 mmol) in DCM (5 mL) was added TFA (1 mL) at 0° C. and the resulting mixture stirred at rt for 1 h. The solvent was removed under reduced pressure and EA (30 mL) and aqueous NaHCO₃ (20 mL) solution was added. The organic layer was washed with brine (20 mL), dried over anhydrous sodium sulfate, filtered and the filtrate concentrated to dryness under reduced pressure to afford 4-8 (150 mg, 67%) as a white solid. ESI-MS m/z: (M+H)⁺=287.2.

Step F: To a solution of 1-9 (100 mg, 0.155 mmol) and PyBOP (161 mg, 0.31 mmol) in DMF (2 mL) was added compound 4-8 (66 mg, 0.233 mmol) with DIEA (80 mg, 0.62 mmol). The mixture was stirred at room temperature for 1 h, then poured into water (50 mL) and extracted with ethyl acetate (50 mL×3). The organic layer was washed with brine (50 mL), dried over anhydrous sodium sulfate, filtered and the filtrate concentrated to dryness under reduced pressure. The residue was purified by C18 column chromatography (FA/H₂O:ACN=45:55) to give 4-9 (100 mg, yield: 70%) as a yellow solid. ESI-MS m/z: (M+H)⁺=914.4.

Step G: A solution of 4-9 (100 mg, 0.04 mmol) in HCl/dioxane (4 mL, 4M) was stirred at 40° C. for 2 h. The solvent was removed under reduced pressure and EA (20 mL) and aq. NaHCO₃ (20 mL) were added. The organic layer was washed with brine (50 mL), dried over anhydrous sodium sulfate, filtered and the filtrate concentrated to dryness under reduced pressure to afford 4-10 (60 mg, 77%) as a yellow solid. ESI-MS m/z: (M+H)⁺=710.1.

Step H: To a solution of 4-11 (137 mg, 1.15 mmol) and pyridine (0.37 mL, 4.6 mmol) in MeCN (4.5 mL) was added BTC (307 mg, 1.03 mmol) at 0° C. under N₂ atmosphere. The reaction mixture was stirred at 0° C. for 2 h, then 1.5 mL of the mixture was added to a solution of 4-10 (50 mg, 0.07 mmol) in DME (1 mL) and pyridine (0.37 mL, 4.6 mmol) at rt under N₂ atmosphere. The crude residue was purified by prep-HPLC to give 109 (18 mg, yield: 30%) as a white solid. ESI-MS m/z: (M+H)⁺=855.4. ¹H NMR (400 MHZ, DMSO-d6) δ 9.27 (s, 1H), 8.10 (s, 2H), 7.79 (s, 1H), 7.35-6.77 (m, 3H), 5.26 (d, J=53.6 Hz, 1H), 4.70-4.60 (m, 2H), 4.48-4.40 (m, 1H), 4.18-4.04 (m, 3H), 3.98-3.92 (m, 1H), 3.54 (d, J=14.4 Hz, 1H), 3.16-2.96 (m, 3H), 2.87-2.76 (m, 1H), 2.35-2.26 (m, 1H), 2.21-1.91 (m, 6H), 1.82-1.69 (m, 3H), 1.58-1.51 (m, 1H), 1.33 (d, J=6.8 Hz, 3H), 1.12 (d, J=6.8 Hz, 3H), 1.05 (d, J=6.8 Hz, 3H).

### Example 1e: Synthesis of (4R)-2-amino-4-(6-chloro-4-(2-(3-chloro-1H-1,2,4-triazole-1-carbonyl)-1-isopropyl-5-oxa-2,8-diazaspiro[3.5]nonan-8-yl)-8-fluoro-2-(((2R,7aS)-2-fluorotetrahydro-1H-pyrrolizin-7a(5H)-yl)methoxy)quinazolin-7-yl)-7-fluorobenzo[b]thiophene-3-carbonitrile (196)

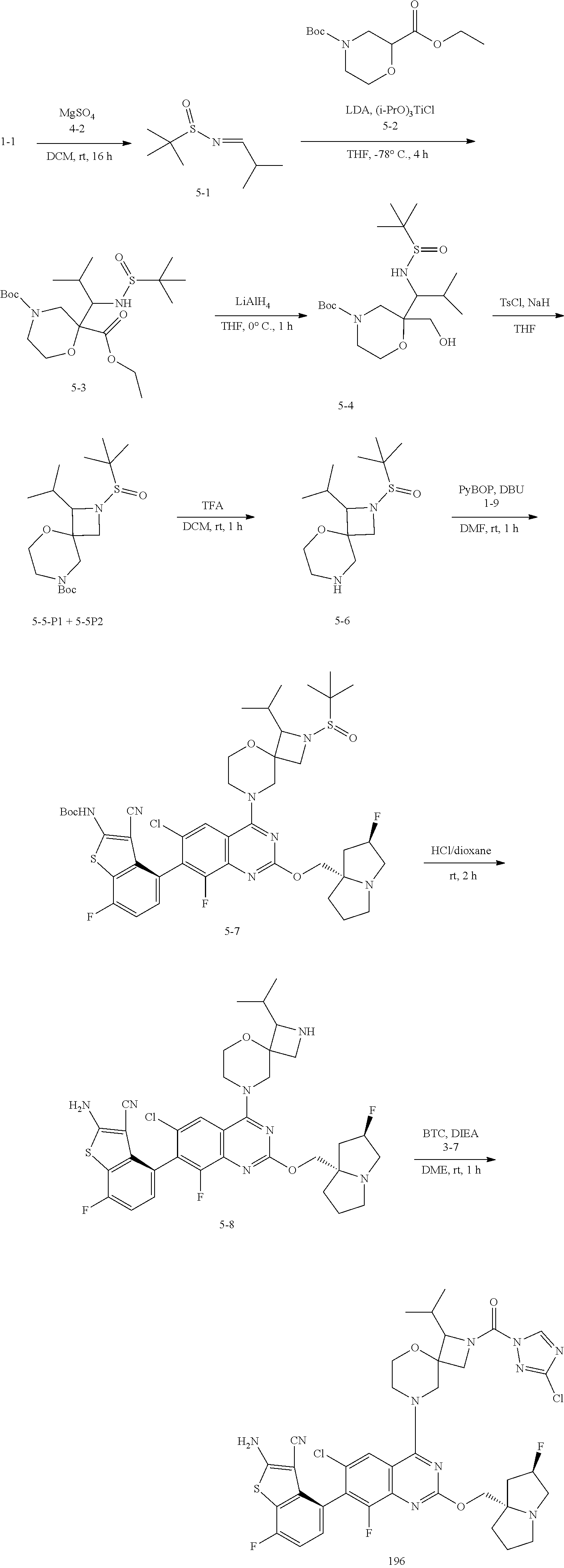

Step A: A mixture of 1-1 (5 g, 41.25 mmol), 4-2 (14.87 g, 206 mmol) and MgSO₄ (24.8 g, 206 mmol) in DCM (50 mL) was stirred at rt for 16 h. The reaction solution was filtered and the organic layer was concentrated to dryness under reduced pressure. The residue was purified by silica gel chromatography (PE/EA=5/1) to give 5-1 (5 g, yield: 69.3%) as a colorless oil. ESI-MS m/z: (M+H)⁺=176.0.

Step B: N-butyllithium (2.5 M in hexanes, 17.17 mL, 42.85 mmol) was added dropwise at 0° C. to a solution of diisopropylamine (4.4 g, 42.85 mmol) in dry THF (30 mL) and the solution was stirred at 0° C. for 0.5 h. The reaction mixture was then cooled to −78° C. followed by dropwise addition of a solution of 5-2 (8.4 g, 34.28 mmol). The resulting mixture was stirred at −78° C. for 1 h, then a solution of (i- PrO)₃TiCl (51.6 mL, 51.6 mmol) was added dropwise and the reaction stirred for 1 h. A solution of 5-1 (8.4 g, 34.28 mmol) in THF (10 mL) was then added dropwise and the reaction stirred for an additional 1 h. The reaction mixture was diluted with NH₄Cl (aq) (70 mL) and extracted with EtOAc (70 mL×3). The organic layer was washed with brine (40 mL), dried over anhydrous sodium sulfate, filtered and the filtrate concentrated to dryness under reduced pressure. The residue was purified by column on silica gel (PE/EA=3/1) to give 5-3 as colorless oil. (2.6 g, yield: 35%). ESI-MS m/z: (M+H)⁺=435.2.

Step C: To a solution of 5-3 (2.6 g, 6 mmol) in dry THF (10 mL) was added LiAlH₄ (9 mL, 9 mmol) at 0° C. The mixture was stirred at 25° C. for 1 h, then 10H₂O·Na₂SO₄ was added. The reaction solution was filtered, dried over anhydrous sodium sulfate, filtered and the filtrate concentrated to dryness under reduced pressure to give 5-4 (1.6 g, yield: 67%) as colorless oil. ESI-MS m/z: (M+H)⁺=392.2.

Step D: To a solution of 5-4 (1.6 g, 4.08 mmol) and TsCl (1.163 g, 6.12 mmol) in dry THF (10 mL) was added NaH (652.8 mg, 16.32 mmol) at 0° C. The mixture was stirred at 25° C. for 3 h, then diluted with NH₄Cl (aq) (20 mL) and extracted with EA (20 mL×3). The organic layer was washed with brine (20 mL), dried over anhydrous sodium sulfate, filtered and the filtrate concentrated to dryness under reduced pressure. The residue was purified by silica gel chromatography (PE/EA=1/1) to give 5-5 as a colorless oil. (P1:560 mg, P2:260 mg yield: 54%). ESI-MS m/z: (M+H)⁺=374.2.

Step E: To a solution of 5-5-P2 (250 mg, 1.5 mmol) in DCM (4 mL) was added TFA (1.7 g, 15 mmol) at 0° C. The mixture was stirred at 25° C. for 1 h. The solvent was removed under reduced pressure to afford 5-6 (130 mg, crude) as a yellow oil. ESI-MS m/z: (M+H)⁺=274.2.

Step F: To a solution of 5-6 (80 mg, 0.29 mmol), 1-9 (150 mg, 0.23 mmol), and PyBOP (242 mg, 0.47 mmol) in DMF (4 mL) was added DIEA (89.2 mg, 0.69 mmol). The mixture was stirred at 25° C. for 1 h, then extracted with ethyl acetate (100 mL×3). The organic layer was washed with brine (100 mL), dried over anhydrous sodium sulfate, filtered and the filtrate concentrated to dryness under reduced pressure. The residue was purified by silica gel chromatography (DCM/MeOH=15/1) to give 5-7 (110 mg, yield: 53%) as a yellow solid. ESI-MS m/z: (M+H)⁺=902.3.

Step G: A solution of 5-7 (110 mg, 0.12 mmol) in HCl/dioxane (4 mL) was stirred at 20° C. for 2 h. The solvent was removed under reduced pressure, then a.q. NaHCO₃ (100 mL) and DCM/MeOH=10/1 (300 mL) were added. The organic layer was washed with brine, dried over sodium sulfate, filtered, and concentrated to give 5-8 (80 mg, yield: 96%) as a yellow solid. ESI-MS m/z: (M+H)⁺=698.2.

Step H: To a solution of 3-7 (516 mg, 5.006 mmol) and DIEA (2.59 g, 20.024 mmol) in 17 mL MeCN, was added BTC (1337 mg, 4.505 mmol) at 0° C. under N₂ atmosphere. The reaction mixture was stirred at rt for 2 h, then 0.16 mL of the mixture was added to a solution of 5-8 (90 mg, 0.13 mmol) and DIEA (50.4 mg, 0.39 mmol) in DMF (2 mL) at rt under N₂ atmosphere. The crude product was purified by prep-HPLC to give 196 (4.50 mg, yield: 4.2%). ¹H NMR (400 MHZ, DMSO-d6) δ 9.22 (s, 1H), 8.04-7.98 (m, J=52.0 Hz, 3H), 7.26 (t, J=5.4 Hz, 1H), 7.15 (t, J=8.9 Hz, 1H), 5.27 (d, J=54.8 Hz, 1H), 4.63-3.51 (m, 11H), 3.14-2.84 (m, 4H), 2.19-1.74 (m, 7H), 1.15-1.00 (m, 6H).

### Example 1f: Synthesis of (4R)-2-amino-4-(6-chloro-8-fluoro-2-(((2R,7aS)-2-fluorotetrahydro-1H-pyrrolizin-7a(5H)-yl)methoxy)-4-(6-methylhexahydropyrrolo[3,4-b]pyrrol-1 (2H)-yl) quinazolin-7-yl)-7-fluorobenzo[b]thiophene-3-carbonitrile (121)

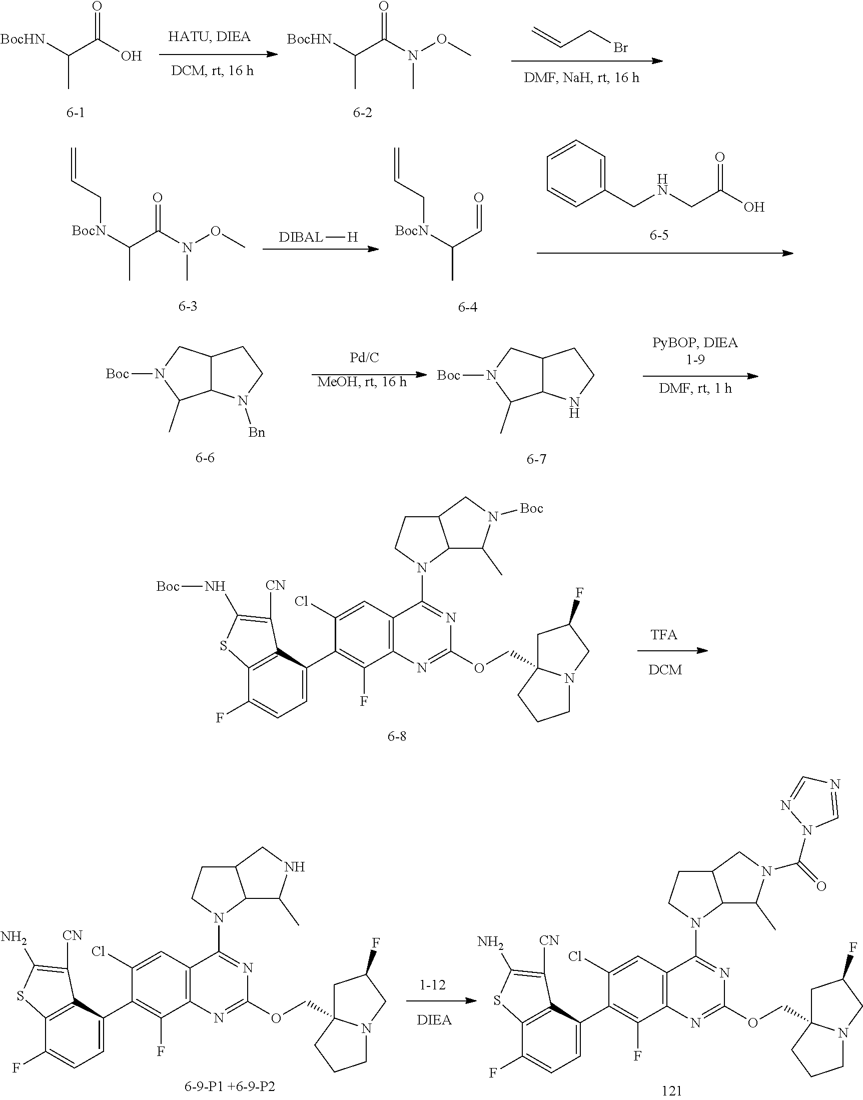

Step A: N-methoxymethylamine hydrochloride (5.60 g, 57.73 mmol) was added at 0° C. to a solution of 6-1 (10.00 g, 52.91 mmol), HATU (21.11 g, 55.55 mmol) and diisopropylethylamine (20 mL) in 200 mL dichloromethane. The resulting mixture was stirred at 25° C. for 16 h, then 200 mL water was added and the pH of the mixture adjusted to 14 with 1 N aqueous sodium hydroxide solution. The product was extracted with dichloromethane (200 mL×2) and the combined organic phase was dried over anhydrous sodium sulfate, filtered and concentrated. The crude product was purified by silica gel column chromatography (PE/EtOAc=100-0%) to give 6-2 (12 g, yield: 98%) as a white solid. ESI-MS m/z: (M+H)⁺=233.2.

Step B: Sodium hydride (3.52 g, 87.93 mol, 60%) was added to a solution of 6-2 (12.00 g, 51.72 mmol) in DMF (200 mL) at 0° C. The mixture was stirred an additional 10 min, then 3-bromopropene (15.65 g, 129.31 mmol) was added dropwise at 0° C. The resulting mixture was reacted at 15° C. for 22 h, then 200 mL saturated ammonium chloride aqueous solution and water (200 mL) were added and the mixture extracted with EtOAc (200 mL×2). The organic phase was washed with saturated brine (300 mL×3) and dried over anhydrous sodium sulfate, filtered, and the filtrate concentrated to dryness under reduced pressure. The crude product was purified by silica gel chromatography (PE/EtOAc=100-0%) to give 6-3 (9 g, yield: 64%) as a transparent oil. ¹H NMR (400 MHZ) 5.86-5.81 (m, 1H), 5.30-5.25 (m, 1H), 5.14-5.04 (m, 2H), 3.95-3.83 (m, 2H), 3.74 (s, 3H), 3.16 (s, 3H), 1.44 (s, 9H) 1.31 (d, J=8.0 Hz, 3H).

Step C: DIBAL-H (66.00 mmol, 66.00 mL, 1 M) was added to a solution of 6-3 (9.00 g, 33.09 mmol) in 200 mL THF at −78° C. under nitrogen. The resultant mixture was stirred at 20° C. for 2 h, then sat'd potassium tartrate (400 mL), water (200 mL) and EtOAc (300 mL) were added and the product extracted in EtOAc (300 mL×2). The organic phase was washed with saturated brine, dried over anhydrous sodium sulfate, filtered and concentrated to give crude 6-4 (7 g, yield: 98%) as a white solid which used directly in the next step without further purification.

Step D: A solution of 6-4 (7.00 g, 32.86 mmol) and 6-5 (13.55 g, 82.15 mmol) in 307 mL toluene was heated to 135° C. and stirred for 24 hrs. Water (300 mL) was then added and the product extracted in EtOAc (300 mL×4). The combined organic phase was washed with saturated brine (300 mL×3), dried over anhydrous sodium sulfate, filtered and concentrated, the crude product obtained was purified by silica gel chromatography (PE/EtOAc=100-0%) to give 6-6 (7 g, yield: 48%) as a white solid. ESI-MS m/z: (M+H)⁺=317.2.

Step E: Pd/C (2.50 g, 10%) was added to a solution of 6-6 (5.00 g, 22.12 mmol) in MeOH (100 mL) and the resulting mixture was purged with nitrogen, then treated with hydrogen and stirred at 20° C. for 16 h. The mixture was then filtered and concentrated, and the crude product was purified by column chromatography (PE/EtOAc=100-0%) to give 6-7 (3 g, yield: 85%) as a white solid. ESI-MS m/z: (M+H)⁺=227.17.

Step F: To a solution of 6-7 (200 mg, 0.88 mmol), 1-9 (570 mg, 0.88 mmol), and PyBOP (1.37 g, 2.64 mmol) in DMF (10 mL) was added DIEA (340.56 mg, 2.64 mmol). The mixture was stirred at 25° C. for 1 h, then extracted with ethyl acetate (100 mL×3). The organic layer was washed with brine (100 mL), dried over anhydrous sodium sulfate, filtered and the filtrate concentrated to dryness under reduced pressure. The residue was purified by silica gel column (DCM/MeOH=15/1) to give 6-8 (90 mg, yield: 12%) as a yellow solid. ESI-MS m/z: (M+H)⁺=854.30.

Step G: To a solution of 6-8 (90.00 mg, 0.11 mmol) in DCM (1 mL) was added TFA (1 mL). The reaction mixture was stirred at 20° C. for 30 min, then solvent was removed under reduced pressure. The reaction mixture was diluted with a.q. NaHCO₃ (20 mL) and DCM/MeOH=10/1 (300 mL), then the organic layer was washed with brine, dried over sodium sulfate, filtered, and concentrated. The residue was purified by silica gel chromatography (PE/EA=1/1) to give 6-9-P1 (30 mg, crude) and 6-9-P2 (20 mg, crude) as a yellow solid. ESI-MS m/z: (M+H)⁺=654.20.

Step H: To a solution of 6-9-P2 (20 mg, 0.031 mmol) and TEA (9.3 mg, 0.092 mmol) in 1 mL DMF was added 1-12 (15 mg, 0.092 mmol) and the resulting mixture stirred at rt for 1 h. The crude product was purified by prep-HPLC to give 121 (1.28 mg, yield: 5.6%). ESI-MS m/z: (M+H)⁺=750.3. ¹H NMR (400 MHZ, DMSO-₆) δ 9.09 (s, 1H), 8.32-8.02 (m, 5H), 7.25-7.18 (m, 1H), 7.14 (t, J=8.8 Hz, 1H), 5.29 (d, J=54.8 Hz, 1H), 4.68-4.41 (m, 3H), 4.15 (d, J=10.0 Hz, 1H), 4.05 (d, J=10.4 Hz, 2H), 3.86-3.66 (m, 2H), 3.10 (d, J=15.2 Hz, 3H), 3.03 (s, 1H), 2.84 (d, J=6.0 Hz, 1H), 2.21-2.00 (m, 5H), 1.89-1.76 (m, 3H), 1.40 (t, J=32.8 Hz, 3H).

### Example 1g: Synthesis of 1-((2R,3R)-3-(((7-((R)-2-amino-3-cyano-7-fluorobenzo[b]thiophen-4-yl)-6-chloro-8-fluoro-2-(((2R,7aS)-2-fluorotetrahydro-1H-pyrrolizin-7a (5H)-yl) methoxy) quinazolin-4-yl)(methyl)amino)methyl)-2-isopropylazetidine-1-carbonyl)-1H-1,2,4-triazole-3-carbonitrile (162)

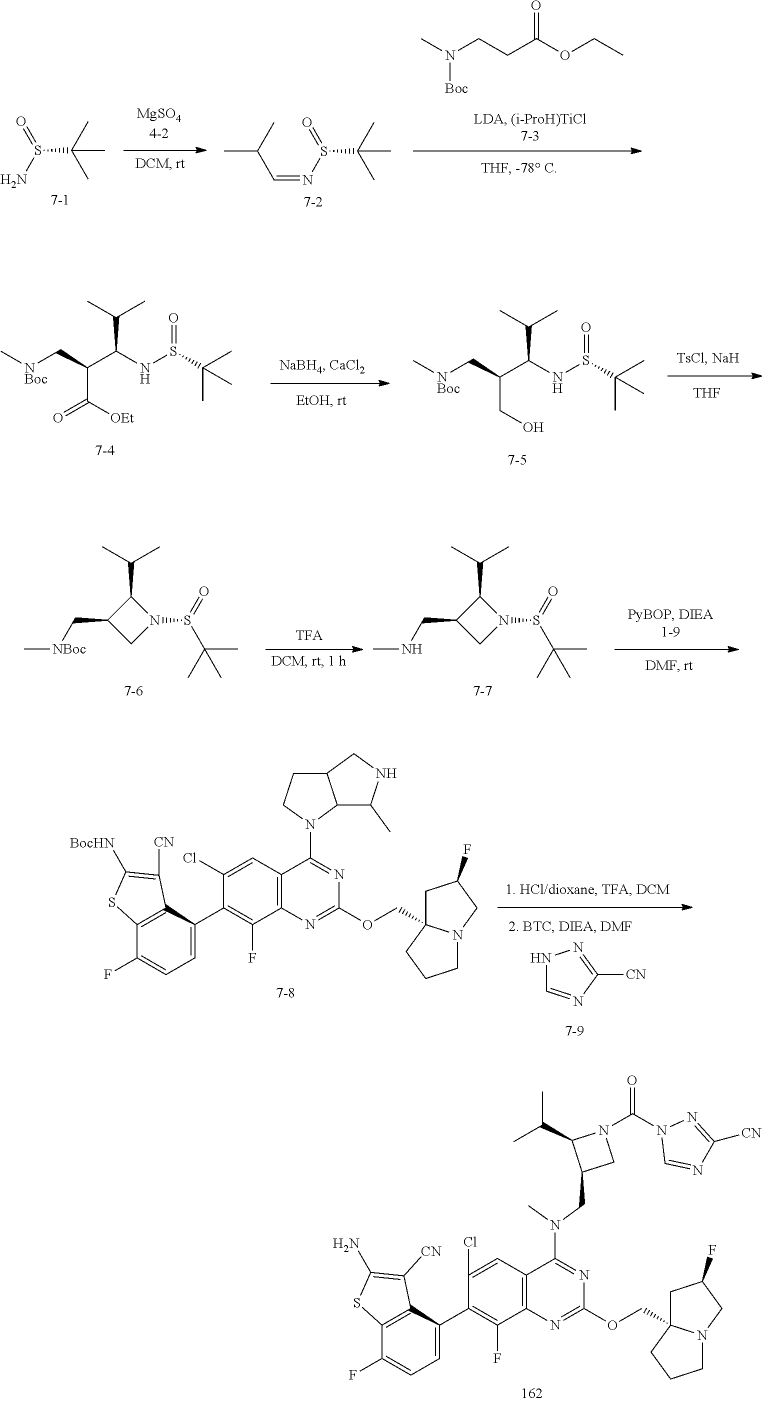

Step A: To a 250 mL round bottom flask was added 7-1 (10 g, 82.5 mmol), 4-2 (17.85 g, 247.5 mmol), MgSO₄ (50 g, 415.4 mmol) and DCM (150 mL). The mixture was stirred for 16 h at room temperature. The crude product was purified by silica gel chromatography eluted with PE:EtOAc=10:1 to give 7-2 (8.5 g, Yield: 58.6%) as a colorless oil.

Step B: n-Butyllithium (2.5 M in hexanes, 28.8 mL, 72 mmol) was added dropwise to a solution of diisopropylamine (7.3 g, 72 mmol) in dry THF (30 mL) at −78° C. and the resulting solution was stirred for 0.5 h. 7-3 (13.2 g, 57.1 mmol) was added and the mixture was stirred for 1 h at −78° C. Next, (i-ProH)₃TiCl (86 mL, 86 mmol) was added and the mixture was stirred an additional 1 h at −78° C., then 7-2 (5 g, 28.5 mmol) was added and the mixture was stirred for another 1 h at −78° C. The mixture was quenched with sat. NH₄Cl (aq.) and extracted with EtOAc (200 mL×2). The organic layer was washed with brine, dried over sodium sulfate, filtered, and concentrated to dryness under reduced pressure to give a crude residue, which was purified by silica gel chromatography eluted with PE:EtOAc=1:1 to give 7-4 (8.4 g, 69.2%) as a yellow oil.

Step C: To a solution of 7-4 (3 g, 5.03 mmol) and CaCl₂) (3.85 g, 36.9 mmol) in EtOH (30 mL) was added NaBH₄ (2.8 g, 74.0 mmol) in portions at 0° C. The mixture was stirred for 16 h at room temperature, then the pH was adjusted to 6 with citric acid (1 M). The resulting mixture was extracted with EtOAc (2×100 mL) and the combined organic layers were washed with brine, dried over sodium sulfate, filtered, and concentrated to dryness under reduced pressure to give a crude residue, which was purified by silica gel chromatography eluted with PE:EtOAc=1:1 to give 7-5 (1.8 g, 66.9%) as a colorless oil.

Step D: To the solution of 7-5 (280 mg, 0.768 mmol) in THF (10 mL) were added TsCl (220 mg, 1.15 mmol) and NaH (123 mg, 3.08 mmol) at 0° C. The mixture was stirred for 16 h at room temperature, then quenched with aqueous NH₄Cl. The filtrate was extracted with EtOAc (50 mL×2). The organic layer was washed with brine, dried over sodium sulfate, filtered, and concentrated to dryness under reduced pressure to give a crude residue, which was purified by silica gel chromatography eluted with PE:EtOAc=3:1 to give 7-6 (180 mg, 67.7%) as a white solid.

Step E: To a solution of 7-6 (160 mg, 0.462 mmol) in DCM (50 mL) was added TFA (1 mL) and the resulting mixture was stirred for 1 h. The mixture was adjusted to pH 7 by the addition of sat. NaHCO₃, then extracted with DCM (50 mL×2). The organic layer was washed with brine, dried over sodium sulfate, filtered, and concentrated to dryness under reduced pressure to give 7-7 (120 mg) as a light-yellow oil.

Step F: To a solution of 7-7 (70 mg, 0.284 mmol), 1-9 (140 mg, 0.217 mmol) and PyBop (338 mg, 0.65 mmol) in DMF (5 ml) was added DIEA (140 mg, 1.08 mmol). The reaction mixture was stirred for 1 h, then extracted with ethyl acetate (10 mL×3). The organic layer was washed with brine, dried over sodium sulfate, filtered, and concentrated to dryness under reduced pressure to give a crude residue, which was purified by prep-TLC (DCM/MeOH=15/1) to give 7-8 (140 mg, yield: 73.7%) as a yellow solid.

Step G: To 7-8 (140 mg, 0.089 mmol) was added HCl/dioxane (3 mL) and the resulting mixture was stirred for 1 h. The mixture was concentrated, then TFA (3 mL) was added and the reaction mixture stirred an additional 1 h. Sat. NaHCO₃ was added to adjust the pH to 7. The mixture was extracted with DCM (50 mL×2) and the combined organic layers were washed with brine, dried over sodium sulfate, filtered, and concentrated to dryness under reduced pressure to give the deprotected product (70 mg) as a yellow solid.

Step H: To a solution of 7-9 (11.5 mg, 0.097 mmol) and pyridine (30 mg, 0.39 mmol) in CAN (0.8 mL) was added BTC (26 mg, 0.087 mmol) at 0° C. and the resulting mixture was stirred for 1 h at room temperature. The mixture was then added to the product from Step G (65 mg, 0.097 mmol) in DME (1 mL) and the resulting mixture was stirred for 10 min. The crude product was purified by prep-HPLC to give 162 (12.11 mg, yield: 15.8%) as a white solid. ESI-MS m/z: (M+H)⁺=790.2. ¹H NMR (400 MHZ, DMSO) δ 9.47 (s, 1H), 8.20-8.02 (m, 3H), 7.30-7.09 (m, 2H), 5.40-5.18 (d, J-52.4 Hz, 1H), 4.60-4.39 (m, 3H), 4.31-4.04 (m, 4H), 3.47 (s, 3H), 3.46-3.35 (m, 1H), 3.18-2.97 (m, 3H), 2.82-2.75 (m, 1H), 2.35-2.25 (m, 1H), 2.21-1.96 (m, 3H), 1.84-1.72 (m, 3H), 1.12-0.96 (m, 6H).

### Example 1h: Synthesis of (R)-2-amino-4-(6-chloro-8-fluoro-4-((1R,4R)-2-(3-fluoro-1H-1,2,4-triazole-1-carbonyl)-1-(1-methylcyclopropyl)-2,6-diazaspiro[3.6]decan-6-yl)-2-(((2R,7aS)-2-fluorotetrahydro-1H-pyrrolizin-7a (5H)-yl) methoxy) quinazolin-7-yl)-7-fluorobenzo[b]thiophene-3-carbonitrile (193)

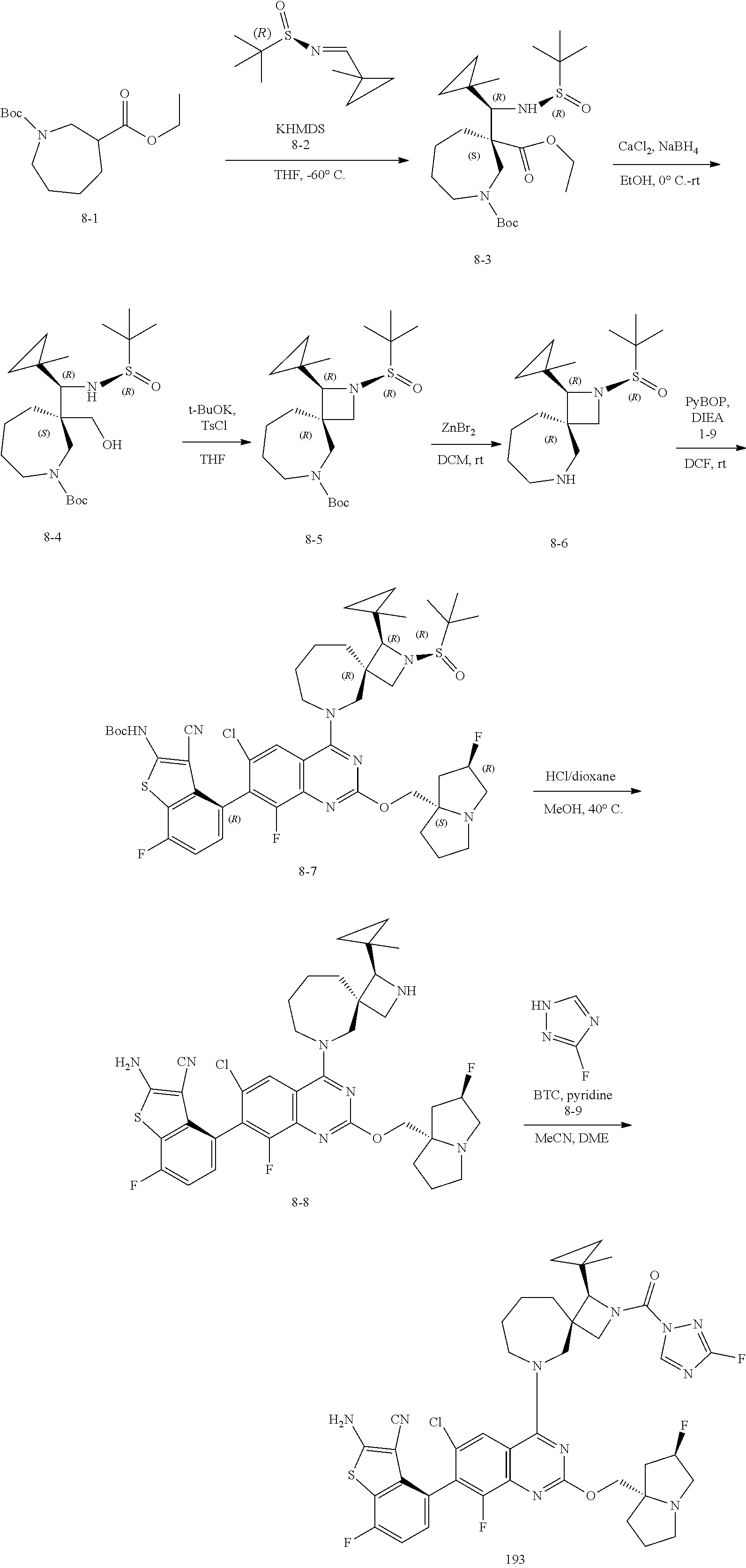

Step A: To a solution of 8-1 (5 g, 18.45 mmol) in THF (30 mL) was added MgCl₂ (1.65 g, 18.45 mmol) and KHMDS (27.5 mL, 27.675 mmol) at −60° C. and the mixture stirred for 1 h. Compound 8-2 (4.15 g, 22.14 mmol) was added, then the mixture was quenched with ice water (40 mL). The aqueous layer was extracted with EtOAc (3×50 mL). The organic layer was washed with brine, dried over sodium sulfate, filtered, and concentrated to dryness under reduced pressure to give a crude residue, which was purified by flash chromatography to afford 8-3 (5.5 g, 65%) as a white solid. ESI-MS m/z: (M+H)⁺=459.1. ¹H NMR (400 MHZ, DMSO) δ 5.21-5.20 (m, 1H), 4.33-4.01 (m, 3H), 3.57-3.56 (m, 1H), 3.36-3.32 (m, 1H), 3.15-3.12 (m, 1H), 2.67-2.57 (m, 1H), 2.24-2.23 (m, 1H), 1.77-1.55 (m, 2H), 1.43-1.33 (m, 11H), 1.23-1.22 (m, 3H), 1.15-1.14 (m, 10H), 0.87-0.85 (m, 4H), 0.37-0.35 (m, 2H), 0.14-0.13 (m, 1H).

Step B: To a solution of 8-3 (1 g, 2.18 mmol) in EtOH (10 mL) were added CaCl₂) (485 mg, 4.37 mmol) and NaBH₄ (332 mg, 8.73 mmol) at 0° C. The mixture was stirred for 16 h at rt, then quenched with citric acid (20 mL/1 g/mL). The aqueous layer was extracted with EtOAc (2×30 mL) and the combined organic layer was washed with brine, dried over sodium sulfate, filtered, and concentrated to dryness under reduced pressure to give a crude residue, which was purified by flash chromatography to afford 8-4 (800 mg, 89%) as a white solid. ESI-MS m/z: (M+H)⁺=417.4. ¹H NMR (400 MHZ, DMSO-d6) δ 6.00-5.95 (m, 1H), 5.20-5.19 (m, 1H), 3.77-3.56 (m, 3H), 3.41-3.39 (m, 1H), 3.09-2.75 (m, 2H), 2.22-2.21 (m, 1H), 1.76-1.51 (m, 5H), 1.40-1.25 (m, 10H), 1.14-1.05 (m, 12H), 0.69-0.65 (m, 1H), 0.40-0.35 (m, 2H), 0.07-0.06 (m, 1H).

Step C: To a solution of 8-4 (1.5 g, 3.597 mmol) in THF (75 mL) were added TsCI (1.37 g, 7.194 mmol) and potassium tert-butoxide (2 g, 17.985 mmol) at 0° C. and the resulting mixture was stirred for 16 h at rt. The reaction mixture was quenched with water (10 mL) and the aqueous layer was extracted with EtOAc (3×30 mL). The organic layer was washed with brine, dried over sodium sulfate, filtered, and concentrated to dryness under reduced pressure to give a crude residue, which was purified by flash chromatography to afford 8-5 (1.3 g, 90%) as a yellow oil. ESI-MS m/z: (M+H)⁺=399.1. ¹H NMR (400 MHZ, DMSO-d6) δ 4.53-4.41 (m, 1H), 3.64-3.41 (m, 3H), 3.19-2.94 (m, 3H), 1.77-1.48 (m, 5H), 1.41-1.35 (m, 9H), 1.32-1.18 (m, 1H), 1.13-0.97 (m, 12H), 0.87-0.85 (m, 1H), 0.41-0.39 (m, 2H), 0.12-0.09 (m, 1H).

Step D: To a solution of 8-5 (500 mg, 1.253 mmol) in DCM (10 mL) was added ZnBr₂ (564 mg, 2.506 mmol). The mixture was stirred for 16 h at 50° C., then quenched by the addition of saturated aqueous NaHCO₃ (15 mL). The aqueous layer was extracted with EtOAc (3×20 mL). The organic layer was washed with brine, dried over sodium sulfate, filtered, and concentrated to dryness under reduced pressure to give a crude residue, which was purified by flash chromatography to afford 8-6 (350 mg, 93%) as a white solid. ESI-MS m/z: (M+H)⁺=299.0. ¹H NMR (400 MHZ, DMSO-d6) δ 3.57-3.55 (m, 1H), 3.43-3.41 (m, 1H), 3.04-3.01 (m, 2H), 2.99-2.96 (m, 1H), 2.89-2.87 (m, 2H), 1.73-1.71 (m, 1H), 1.69-1.43 (m, 5H), 1.34-1.29 (m, 12H), 0.71-0.70 (m, 1H), 0.32-0.31 (m, 2H), 0.13-0.12 (m, 1H).

Step E: To a solution of compound 1-9 (9.0 g, 14.0 mmol) and PyBOP (10.9 g, 20.9 mmol) in DMF (100 mL) were added 8-6 (5.2 g, 17.4 mmol) and DIEA (3.6 g, 27.9 mmol). The mixture was stirred at room temperature for 1 h, then poured into water (200 mL) and extracted with ethyl acetate (250 mL×3). The organic layer was washed with brine, dried over sodium sulfate, filtered, and concentrated to dryness under reduced pressure to give a crude residue, which was purified by C18 chromatography (FA/H₂O:ACN=45:55) to give 8-7 (8.5 g, yield: 66%) as a yellow solid. ESI-MS m/z: (M+H)⁺=926.1.

Step F: A solution of 8-7 (8.5 g, 9.19 mmol) in HCl/dioxane (85 ml, 4M) was stirred at 40° C. for 2 h. The solvent was removed under reduced pressure, then EtOAc (400 mL×2) and aq. NaHCO₃ (450 mL) were added. The organic layer was washed with brine, dried over sodium sulfate, filtered, and concentrated to dryness under reduced pressure to give a crude residue, which was purified by prep-TLC (DCM/NH₃:MeOH (10:1) to afford 8-8 (3.7 g, 66%) as a yellow solid. ESI-MS m/z: (M+H)⁺=722.1.

Step G: To a solution of 8-9 (446 mg, 5.13 mmol) and pyridine (1.7 ml, 20.5 mmol) in MeCN (40 ml) was added BTC (1.37 g, 4.62 mmol) at 0° C. The reaction mixture was then stirred at 0° C. for 2 h, then added with pyridine (1.7 ml, 20.5 mmol) to a solution of 8-8 (3.7 g, 5.13 mmol) in DME (30 mL) at 30° C. The mixture was poured into water (100 mL), then extracted with ethyl acetate (80 mL×3). The organic layer was washed with brine, dried over sodium sulfate, filtered, and concentrated to dryness under reduced pressure to give a crude residue, which was purified by prep-HPLC (FA) to give 193 (1.5 g, yield: 35%) as a white solid. ESI-MS m/z: (M+H)⁺=835.5. ¹H NMR (400 MHZ, DMSO-d₆) δ 9.07 (s, 1H), 8.19-8.01 (m, 3H), 7.28-7.21 (m, 1H), 7.19-7.12 (m, 1H), 5.23 (d, J=55.6 Hz, 1H), 4.95-4.82 (m, 1H), 4.68-4.56 (m, 1H), 4.28-4.02 (m, 4H), 3.92-3.81 (m, 2H), 3.75-3.66 (m, 1H), 3.15-3.00 (m, 3H), 2.87-2.77 (m, 1H), 2.12-1.72 (m, 9H), 1.71-1.57 (m, 3H), 1.28-1.12 (m, 3H), 0.82-0.68 (m, 1H), 0.61-0.44 (m, 3H).

Example 1i: Synthesis of (R)-2-amino-4-(6-chloro-4-((1R,4R,7S)-2-(3-(difluoromethyl)-1H-1,2,4-triazole-1-carbonyl)-7-methyl-1-(1-methylcyclopropyl)-2,6-diazaspiro[3.5]nonan-6-yl)-8-fluoro-2-(((2R,7aS)-2-fluorotetrahydro-1H-pyrrolizin-7a (5H)- yl)methoxy)quinazolin-7-yl)-7-fluorobenzo[b]thiophene-3-carbonitrile (178).

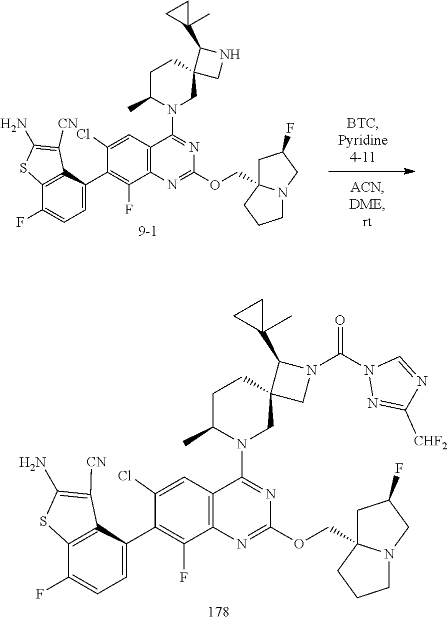

Step A: To a solution of 4-11 (8.2 mg, 0.069 mmol) and pyridine (22 mg, 0.28 mmol) in CAN (0.8 mL) was added BTC (19 mg, 0.062 mmol) at 0° C. The mixture was stirred for 1 h at room temperature, then was added to 9-1 (50 mg, 0.069 mmol) in DME (1 mL). The resulting mixture was stirred for 10 min at room temperature. The crude product was purified by prep-HPLC to give 178 (20.9 mg, yield: 34.8%) as a white solid. ESI-MS m/z: (M+H)⁺=867.4. ¹H NMR (400 MHZ, DMSO) δ 9.27 (s, 1H), 8.10 (s, 2H), 7.82 (s, 1H), 7.27-7.11 (m, 3H), 5.35-5.21 (d, J-54.8 Hz, 1H), 5.16-4.96 (m, 1H), 4.81-4.72 (m, 1H), 4.58-4.40 (m, 1H), 4.22- 4.08 (m, 3H), 4.90-3.78 (s, 1H), 3.75-3.66 (m, 1H), 3.17-2.98 (m, 3H), 2.83-2.75 (m, 1H), 2.25-1.70 (m, 9H), 1.65-1.20 (m, 7H), 0.82-0.43 (m, 4H).

### Example 1j: Synthesis of 1-((2R,3R)-3-(((7-((R)-2-amino-3-cyano-7-fluorobenzo[b]thiophen-4-yl)-6-chloro-8-fluoro-2-(((2R,7aS)-2-fluorotetrahydro-1H-pyrrolizin-7a (5H)-yl) methoxy) quinazolin-4-yl)(methyl)amino)methyl)-2-cyclobutylazetidine-1-carbonyl)-1H-1,2,4-triazole-3-carbonitrile (150)

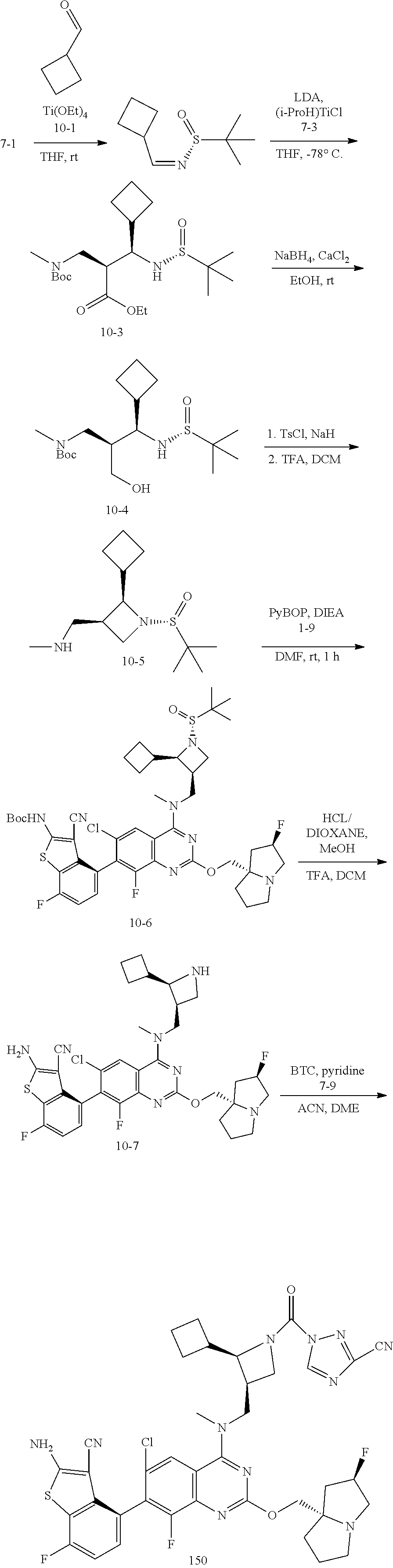

Step A: To a solution of 7-1 (10 g, 82.6 mmol) and 10-1 (14 g, 165.2 mmol) in THF (100 mL) was added Ti(OEt)₄ (57 g, 247.8 mmol) at rt. The mixture was stirred at rt for 16 h, then diluted with Na₂SO₄·10H₂O (10 g) and filtered. The solid was washed with EtOAc (2×300 mL) and the combined organic layers were washed with brine, dried over sodium sulfate, filtered, and concentrated to dryness under reduced pressure to give a crude residue, which was purified by silica gel chromatography (PE:EtOAc=90:10) to provide 10-2 (15 g, yield: 97%) as a colorless oil. ESI-MS m/z: (M+H)⁺=188.0.

Step B: To a solution of diisopropylamine (9.36 ml, 66.8 mmol) in THF (50 ml) was added n-BuLi (41.75 ml, 66.8 mmol, 1.6 M) at −50° C. The reaction mixture was stirred at −50° C. for 45 mins, then added to a solution of 7-3 (9.26 g, 40.0 mmol) in THF (10 ml) at −78° C. After stirring at −78° C. for 1 h, a solution of (i-Pro)₃TiCl (80 mL, 80 mmol, 1M) was added and the resulting mixture stirred an additional 1 h at −78° C. Next, a solution of 10-2 (5.0 g, 26.7 mmol) in THF (10 mL) was added at −78° C. and the mixture stirred an additional 1 h at −78° C. The reaction mixture was diluted with a.q. NH₄Cl (200 mL) and EtOAc (200 mL), then filtered with diatomite. The organic layer was washed with brine, dried over sodium sulfate, filtered, and concentrated to dryness under reduced pressure to give a crude residue, which was purified by silica gel chromatography (PE:EtOAc=70:30) to give 10-3 (7.4 g, yield: 65%) as a white solid. ESI-MS m/z: (M+H)⁺=419.

Step C: To a solution of 10-3 (2 g, 4.7 mmol) in dry EtOH (20 mL) were added CaCl₂) (2.7 g, 24.3 mmol,) and NaBH₄ (1.81 g, 47.8 mmol) at 0° C. The mixture was stirred at 35° C. for 16 h, then quenched with aqueous citric acid (20 mL), water (50 mL) and EtOAc (50 mL). The organic layer was washed with brine, dried over sodium sulfate, filtered, and concentrated to dryness under reduced pressure to give a crude residue, which was purified by silica gel chromatography (PE:EtOAc=60:40) to give 10-4 (1.7 g, yield: 95%) as a white solid. ESI-MS m/z: (M+H)⁺=417.4.

Step D: To a solution of 10-4 (1 g, 2.65 mmol) and TsCl 757 mg, 3.97 mmol) in dry THF (10 mL) was added NaH (255 mg, 10.6 mmol, 60%) at 0° C. The mixture was stirred at 35° C. for 16 h, then diluted with aqueous NH₄Cl (20 mL) and extracted with EtOAc (2×20 mL). The organic layer was washed with brine, dried over sodium sulfate, filtered, and concentrated to dryness under reduced pressure to give a crude residue, which was purified by silica gel chromatography (PE/EtOAc=3/1) to give tert-butyl (((2R,3R)-1-((R)-tert-butylsulfinyl)-2-cyclobutylazetidin-3-yl)methyl)(methyl) carbamate as a white solid. (500 mg, yield: 52%). ESI-MS m/z: (M+H)⁺=359.

Step E: To a solution of tert-butyl (((2R,3R)-1-((R)-tert-butylsulfinyl)-2-cyclobutylazetidin-3-yl)methyl)(methyl) carbamate (500 mg, 1.1 mmol) in DCM (6 mL) was added TFA (1 mL) at 0° C. The mixture was stirred at 25° C. for 1 h. The solvent was removed under reduced pressure to afford 10-5 (240 mg, crude) as a yellow oil. ESI-MS m/z: (M+H)⁺=259.

Step F: To a solution of 1-9 (400 mg, 0.619 mmol) and PyBop (966 mg, 1.85 mmol) in DMF (2 mL) were added 10-5 (207 mg, 0.80 mmol) and DIEA (400 mg, 3.09 mmol). The mixture was stirred at room temperature for 1 h, then poured into water (20 mL) and extracted with ethyl acetate (3×20 mL). The organic layer was washed with brine, dried over sodium sulfate, filtered, and concentrated to dryness under reduced pressure to give a crude residue, which was purified by C18 chromatography (DCM:NH₃:MeOH=10:1) to provide 10-6 (230 mg, Yield: 41%) as a yellow solid. ESI-MS m/z: (M+H)⁺=886.

Step G: Compound 10-6 (280 mg, 0.31 mmol) was dissolved in HCl/dioxane (17 mL, 4M). The reaction mixture was stirred at 45° C. for 1 h. The solvent was removed under reduced pressure, then EtOAc (20 ml) and aq. NaHCO₃ (20 mL) were added. The organic layer was washed with brine, dried over sodium sulfate, filtered, and concentrated to dryness under reduced pressure to give a crude residue, which was purified by prep-TLC (DCM/NH₃·MeOH (10:1) to afford 10-7 (120 mg, 56%) as a yellow solid. ESI-MS m/z: (M+H)⁺=682.3.

Step H: To a solution of 7-9 (4.1 mg, 0.035 mmol) and pyridine (11 mg, 0.14 mmol) in CAN (0.8 mL) was added BTC (10 mg, 0.031 mmol) at 0° C. and the resulting mixture was stirred for 1 h at room temperature. Then, the mixture was added to 10-7 (25 mg, 0.035 mmol) in DME (1 mL) and the resulting mixture was stirred for 10 min at room temperature. The crude product was purified by prep-HPLC to give 150 (5.28 mg, yield: 18%) as a white solid. ESI-MS m/z: (M+H)⁺=802.3. ¹H NMR (400 MHZ, DMSO) δ 9.48 (s, 1H), 8.20-8.05 (m, 3H), 7.27-7.11 (m, 2H), 5.46-5.18 (d, J-53.6 Hz, 1H), 4.78-4.69 (m, 1H), 4.65-4.47 (m, 2H), 4.22-4.06 (m, 3H), 3.98-3.90 (m, 1H), 3.70-3.62 (m, 1H), 3.48 (s, 3H), 3.22-2.78 (m, 5H), 2.26-1.98 (m, 7H), 1.96-1.70 (m, 5H).

### Example 1k: Synthesis of (4R)-2-amino-4-(6-chloro-4-(5-(3-(difluoromethyl)-1H-1,2,4-triazole-1-carbonyl)-4-isopropylhexahydropyrrolo[3,4-b]pyrrol-1 (2H)-yl)-8-fluoro-2-(((2R,7aS)-2-fluorotetrahydro-1H-pyrrolizin-7a (5H)-yl)methoxy)quinazolin-7-yl)-7-fluorobenzo[b]thiophene-3-carbonitrile (214)

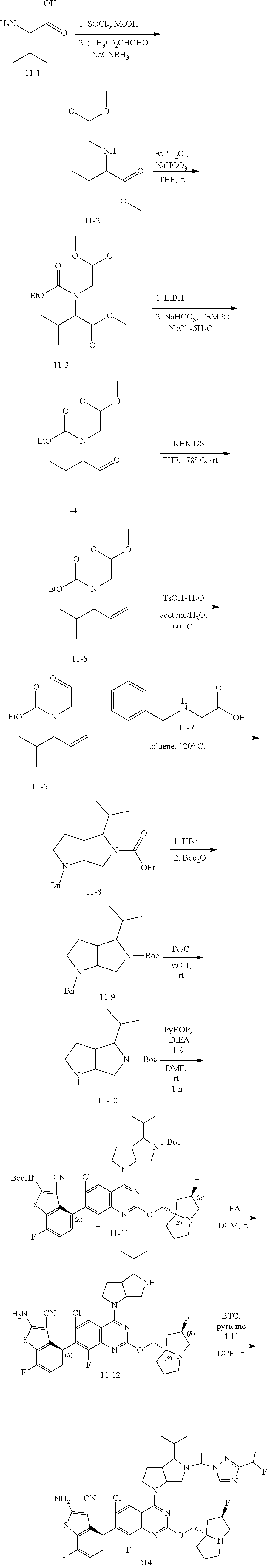

Step A: To a solution of 11-1 (10 g, 85.36 mmol) in MeOH (100 mL) was added SOCl₂ (18 mL, 247.54 mmol) at 0° C. The mixture was stirred at rt for 16 h, then Et₂O was added and the mixture filtered and washed with Et₂O. The filter cake was concentrated to give methyl valinate hydrochloride (13 g, 100.1% yield) as a white solid. ESI-MS m/z: (M+H)⁺=132.2.

Step B: To a solution of methyl valinate hydrochloride (13.42 g, 128.83 mmol) in MeOH (130 mL) was added 2,2-dimethoxyacetaldehyde (13 g, 128.83 mmol) at 0° C. The mixture was stirred at rt for 2 h, then NaCNBH₃ (8.095 g, 128.83 mmol) was added at 0° C. The mixture was stirred at rt for 18 h. Diatomite was added, then the mixture was filtered and the filtrate concentrated to give a residue. The residue was diluted with H₂O (80 mL) and extracted with DCM (200 mL). The combined organic layers were concentrated to give 11-2 (12.5 g, 55% yield) as a yellow solid. ESI-MS m/z: (M+H)⁺=220.2.

Step C: To a solution of 11-2 (5 g, 22.8 mmol) in THF/H₂O (20 mL/20 mL) was added NaHCO₃ (2.872 mg, 34.2 mmol) at rt. Ethyl carbonochloridate (2.969 g, 27.362 mmol) was then added at −5° C.˜5° C. and the resulting mixture was stirred at rt for 2 h. The reaction mixture was diluted with H₂O (80 mL) and extracted with EtOAc (200 mL), then washed twice with KHSO₄ (aq). The combined organic layers were concentrated to give 11-3 (4.8 g, 75% yield) as a yellow oil. ESI-MS m/z: (M+H)⁺=276.1.

Step D: To a solution of 11-3 (5.7 g, 19.564 mmol) in EtOH/THF (90 mL/10 mL) was added LiBH₄ (97.8 mL, 195.64 mmol) at 0° C. The mixture was stirred at rt for 16 h, then diluted with H₂O (80 mL) and extracted with EtOAc (200 mL). The combined organic layers were concentrated to give a residue, which was purified by silica gel chromatography (PE:EtOAc=5:1) to give ethyl (2,2-dimethoxyethyl) (1-hydroxy-3-methylbutan-2-yl) carbamate (2.9 g, 56% yield) as a yellow oil. ESI-MS m/z: (M+H)⁺=263.2.

Step E: To a solution of ethyl (2,2-dimethoxyethyl) (1-hydroxy-3-methylbutan-2-yl) carbamate (5.9 g, 22.405 mmol) in DCM/H2O (25 mL/25 mL) were added NaHCO₃ (5.645 g, 67.214 mmol) and TEMPO (699 mg, 4.4808 mmol) at rt. Then, 1M NaOCl·5H₂O (60 mL, 113.358 mmol) was added at rt and the resulting mixture was stirred at rt for 18 h. The reaction mixture was diluted with sodium thiosulfate (aq, 80 mL) and extracted with DCM (200 mL). The combined organic layers were concentrated to give a residue, which was purified by silica gel chromatography (PE:EtOAc=4:1) to give 11-4 (1.98 g, 34% yield) as a yellow oil. ESI-MS m/z: (M+H)⁺=261.3.

Step F: KHMDS (4.5 mL, 4.477 mmol) was added to a −78° C. solution of 11-4 (900 mg, 3.444 mmol) in THF (5 mL) and stirred at −78° C. for 1 h, then at rt for 16 hours. The mixture was quenched with MeOH at rt for 30 min, then concentrated and suspended in normal hexane at 0° C., filtered and concentrated in vacuo to provide 11-5 (396 mg, 44.3% yield) as a yellow oil. ESI-MS m/z: (M+H)⁺=245.2.

Step G: To a solution of 11-5 (696 mg, 2.683 mmol) in acetone/H₂O (10 mL/1 mL) was added TsOH·H₂O (163.34 mg, 0.857 mmol) at rt. The mixture was stirred at 60° C. for 18 h, then diluted with H₂O (80 mL) and extracted with EtOAc (200 mL). The combined organic layers were concentrated to give a residue, which was purified by silica gel chromatography (PE:EtOAc=4:1) to give 11-6 (634 mg, 80% yield) as a yellow oil. ESI-MS m/z: (M+H)⁺=213.4.

Step H: A solution of 11-6 (300 mg, 1.407 mmol) and 11-7 (278.95 mg, 1.688 mmol) in toluene (11 mL) was heated to 120° C. and stirred for 18 h. The reaction mixture was diluted with H₂O (80 mL) and extracted with EtOAc (200 mL). The combined organic layers were concentrated to give a residue, which was purified by silica gel chromatography (PE:EtOAc=4:1) to give 11-8 (265 mg, 67% yield) as a yellow oil. ESI-MS m/z: (M+H)⁺=316.3.

Step I: A solution of 11-8 (65 mg, 0.2054 mmol) in HBr (0.5 mL) at rt was stirred at 100° C. for 16 h. The mixture was adjusted to pH 10 with aqueous K₂CO₃, then diluted with H₂O (80 mL) and extracted with DCM (200 mL). The combined organic layers were concentrated to give a residue, which was purified by silica gel chromatography (DCM:MeOH=20:1) to give 1-benzyl-4-isopropyloctahydropyrrolo[3,4-b]pyrrole (71 mg, 100.4% yield) as a yellow oil. ESI-MS m/z: (M+H)⁺=244.2.

Step J: To a solution of 1-benzyl-4-isopropyloctahydropyrrolo[3,4-b]pyrrole (219 mg, 0.8961 mmol) in THF/H₂O (7 mL/8.75 mL) were added NaHCO₃ (2.68 mg, 225.84 mmol) and Boc₂O (469.4 mg, 2.15 mmol) at 0° C. The mixture was stirred at room temperature for 16 h, then filtered with a Buchner funnel and diluted with EtOAc (100 mL). The organic layer was separated and concentrated in vacuo to provide a residue, which was purified by flash chromatography (PE:EtOAc=5:1) to give 11-9 (238 mg, 76.9%) as a colorless liquid. ESI-MS m/z: (M+H)⁺=344.5.

Step K: A solution of 11-9 (100 mg, 0.29 mmol) and Pd/C (200 mg) in EtOH (10 mL) was charged with H₂. The mixture was stirred at rt for 4 h, then filtered and concentrated to give 11-10 (84 mg, 100.1% yield). ESI-MS m/z: (M+H)⁺=254.4.

Step L: A mixture of 11-10 (76.5 mg, 0.3 mmol) and DIEA (0.2 mL) in DMF (3.0 mL) was stirred at rt for 5 minutes, then 1-9 (150 mg, 0.23 mmol) and PyBop (180 mg, 0.345 mmol) were added and the reaction stirred at rt for 1 h. The reaction was diluted with EtOAc and washed with sat. NaCl. The organic layer was removed in vacuo and purified by prep-TLC (DCM:MeOH=15:1) to give 11-11 (180 mg, yield: 82%). ESI-MS m/z: (M+H)⁺=882.3.

Step M: To a solution of 11-11 (110 mg, 0.124 mmol) in DCM (2.0 mL) was added TFA (0.4 mL) at rt and the resulting mixture was stirred at rt for 1 h. The mixture was concentrated in vacuo, then diluted with DCM:MeOH=10:1 and adjusted to pH 8 with sat. NaHCO₃. The organic solvent was concentrated in vacuo and the residue was purified by prep-TLC (DCM:NH₃/MeOH=10:1) to give 11-12 (90 mg, yield: 100%). ESI-MS m/z: (M+H)⁺=682.3.

Step N: To a solution of 4-11 (11.3 mg, 0.11 mmol) and BTC (29 mg, 0.099 mmol) in MeCN (0.3 mL) was added pyridine (35 mg, 0.44 mmol) and the resulting solution was stirred at 0° C. for 5 minutes, then at r.t for 2 h. The mixture was then added to a solution of 11-12 (75 mg, 0.11 mmol) in DME (0.6 mL) and stirred at 30° C. for 10 min. The mixture was concentrated and purified by prep-HPLC (FA) to give 214 (9.22 mg, yield: 10.14%) as a white solid. ESI-MS m/z: (M+H)⁺=827.3. ¹H NMR (400 MHZ, DMSO-d₆) δ 9.27 (d, J=57.6 Hz, 1H), 8.15 (t, J=41.6 Hz, 3H), 7.26-6.96 (m, 3H), 5.27 (d, J=54.4 Hz, 1H), 4.95 (s, 1H), 4.31-3.92 (m, 7H), 3.08 (s, 2H), 3.00 (s, 1H), 2.86-2.80 (m, 1H), 2.35-1.35 (m, 10H), 1.02-0.80 (m, 6H).

### Example 1l: Synthesis of N-((2R,3R)-1-(7-((R)-2-amino-3-cyano-7-fluorobenzo[b]thiophen-4-yl)-6-chloro-8-fluoro-2-(((2R,7aS)-2-fluorotetrahydro-1H-pyrrolizin-7a (5H)-yl) methoxy) quinazolin-4-yl)-2-methylpyrrolidin-3-yl)-3-chloro-N-ethyl-1H-1,2,4-triazole-1-carboxamide (176)

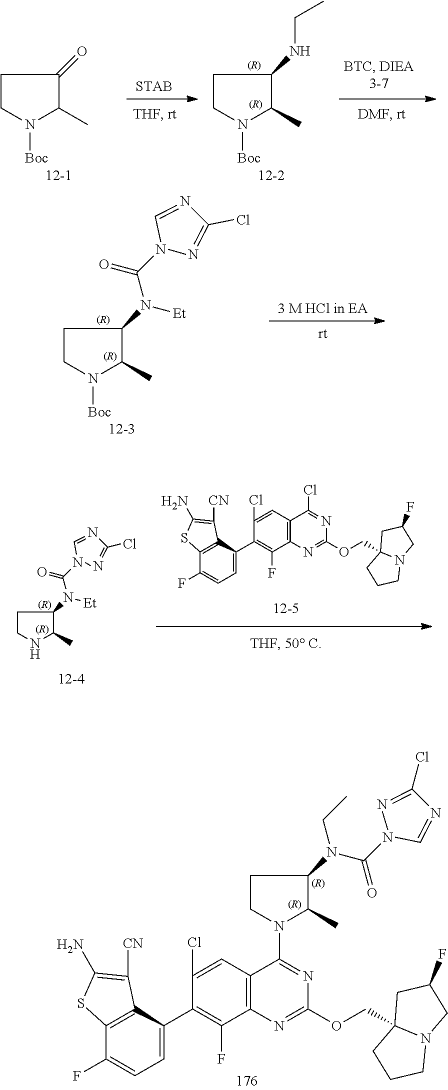

Step A: A mixture of 12-1 (2 g, 10 mmol), ethanamine (10 mL, 20 mmol) and 4 Å molecular sieves (400 mg) in THF (20 mL) was stirred for 1 h. Sodium triacetoxyborohydride (4.26 mg, 10 mmol) was added to the mixture at r.t and the resulting mixture stirred for 16 h, then diluted with EtOAc, basified with NaHCO₃ solution, and washed with water. The organic solvent was removed in vacuo and the residue was purified by silica gel chromatography to give 12-2 (750 mg, yield: 32.9%) as a yellow oil, then further purified by SFC to give 12-2-P1 (400 mg) and 12-2-P2 (350 mg). ESI-MS m/z: (M+H)⁺=229.2. ¹H NMR (400 MHZ, DMSO) δ 3.85-3.71 (m, 1H), 3.33-2.99 (m, 4H), 2.58-2.53 (m, 1H), 1.96-1.90 (m, 1H), 1.59-1.38 (m, 1H), 1.39 (s, 9H), 1.03-0.99 (m, 3H), 0.95 (d, J=5.6 Hz, 3H).

Step B: To a solution of 3-7 (258 mg, 2.53 mmol) and DIEA (1.7 mL, 9.78 mmol) in 8.5 mL MeCN was added BTC (667 mg, 2.25 mmol) at 0° C. The reaction mixture was stirred at rt for 2 h, then added to a mixture of 12-2 (200 mg, 0.88 mmol) and DIEA (0.30 mL, 1.62 mmol) in DMF (2.0 mL) at r.t. The reaction mixture was stirred for 0.5 h, then diluted with sat. NaCl and extracted with EtOAc (2×100 mL). The organic layer was dried over sodium sulfate, filtered, and concentrated to dryness under reduced pressure to give a crude residue, which was purified by silica gel chromatography, eluted with EtOAc in PE (0˜50%) to afford 12-3 (200 mg, yield: 63.7%) as a yellow oil. ESI-MS m/z: (M+23)⁺=380.0. ¹H NMR (400 MHZ, DMSO) δ 9.20 (s, 1H), 4.44-4.31 (m, 1H), 4.23-4.11 (m, 1H), 3.67-3.65 (m, 1H), 3.49-3.34 (m, 2H), 3.39-3.19 (m, 1H), 2.43-2.29 (m, 1H), 2.21-2.11 (m, 1H), 1.40 (s, 9H), 1.19-1.15 (m, 3H), 1.03 (d, J=6.4 Hz, 3H).

Step C: A solution of 12-3 (50 mg, 0.19 mmol) in 3M HCl in EtOAc (5.0 mL) was stirred at rt for 1 h. The solvent was removed in vacuo to provide crude 12-4 (60 mg), which was used directly in the next step without further purification. ESI-MS m/z: (M+H)⁺=258.1.

Step D: To a solution of 12-4 (55 mg, 0.212 mmol) in THF (3.0 mL) was added 12-5 (55 mg, 0.098 mmol) and the resulting mixture was stirred at 40° C. for 16 hr. The reaction mixture was purified by prep-HPLC (FA) to afford 176 (12.5 mg, yield: 16%). ESI-MS m/z: (M+H)⁺=785.6. ¹H NMR (400 MHZ, DMSO) δ 9.26 (s, 1H), 8.41 (s, 1H), 8.15 (d, J=15.6 Hz, 3H), 7.24-7.12 (m, 2H), 5.41-5.06 (m, 2H), 4.49 (d, J=74.4 Hz, 2H), 4.09-4.00 (m, 3H), 3.76 (s, 1H), 3.54 (d, J=7.8 Hz, 1H), 3.08 (d, J=7.8 Hz, 2H), 3.00 (s, 1H), 2.82 (d, J=5.4 Hz, 1H), 2.63 (s, 1H), 2.44 (d, J=7.0 Hz, 1H), 2.06 (t, J=21.6 Hz, 3H), 1.88-1.73 (m, 3H), 1.32-1.27 (m, 6H).

### Example 1m: Synthesis of N-(1-(7-((R)-2-amino-3-cyano-7-fluorobenzo[b]thiophen-4-yl)-6-chloro-8-fluoro-2-(((2R,7aS)-2-fluorotetrahydro-1H-pyrrolizin-7a (5H)-yl) methoxy) quinazolin-4-yl)-2,2-dimethylpyrrolidin-3-yl)-3-chloro-N-methyl-1H-1,2,4-triazole-1-carboxamide (198)

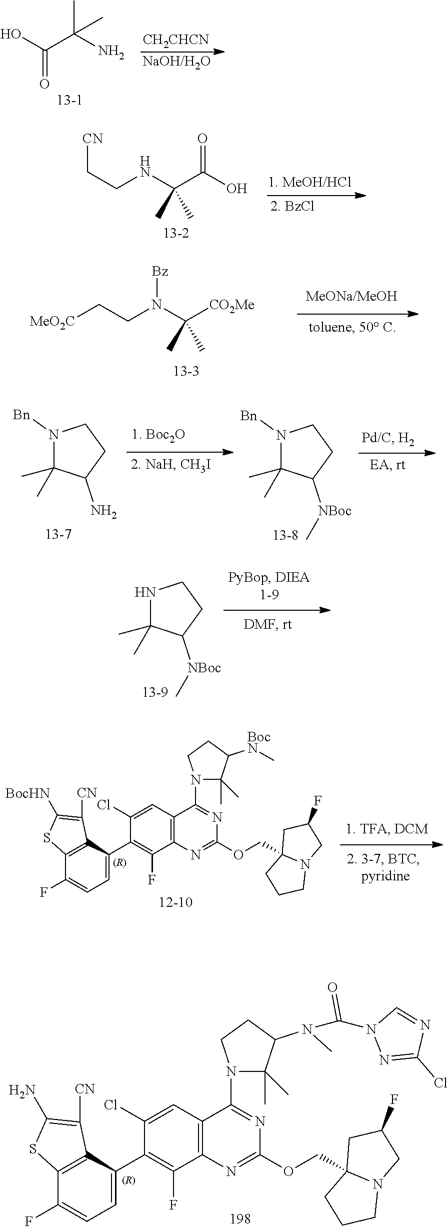

Step A: 13-1 (20 g, 194.2 mmol) was added to a cooled solution (water/ice) of NaOH (7.84 g) in water. Once the solution had turned clear, acrylonitrile (14 mL, 213.6 mmol) was added dropwise with cooling. The mixture was left overnight at rt, then 28 mL of acetic acid was added under ice cooling. A white solid precipitated from the mixture, then 20 mL of ethanol was added and the mixture stirred for 1 hour. The reaction mixture was filtered and the solid was collected and dried. The residue was purified by silica gel chromatography to give 13-2 (31.9 g, yield: 105.3%) as a colorless oil. ESI-MS m/z: (M+H)⁺=156.2.

Step B: A mixture of 13-2 (500 mg, 3.205 mmol) in MeOH/HCl (15 mL) was stirred for 12 h at 75° C. Trichloromethane was added, then the mixture was filtered and the organic layer was dried over sodium sulfate, filtered, and concentrated to dryness under reduced pressure to give a crude residue. The residue was purified by flash chromatography to afford methyl 2-((3-methoxy-3-oxopropyl)amino)-2-methylpropanoate (410 mg, yield: 34%) as a colorless oil. ESI-MS m/z: (M+H)⁺=203.4.

Step C: To a solution of methyl 2-((3-methoxy-3-oxopropyl)amino)-2-methylpropanoate (284 mg, 1.41 mmol) in DCM and H₂O (8 mL) were added NaHCO₃ (237 mg, 2.82 mmol) and benzoyl chloride (208.1 mg, 1.48 mmol). The mixture was vigorously stirred overnight, then the phases were separated. The organic phase was dried over anhydrous MgSO₄, filtered, and concentrated in vacuo. The residue was purified by silica gel chromatography to give 13-3 (280 mg, yield: 69%) as a colorless oil. ESI-MS m/z: (M+H)⁺=307.1.

Step D: To a solution of 13-3 (200 mg, 0.651 mmol) in toluene (6.5 mL) were added MeOH (0.1 mL, 1.303 mmol) and NaOMe (71 mg, 1.303 mmol). The mixture was heated at 60° C. and stirred for 2 h, then an additional portion of MeOH (0.8 mL) was added and the mixture was stirred for an additional 2 h at 60° C. The mixture was then concentrated in vacuo and the residue partitioned between DCM (20 mL) and 1 N aq.HCL (10 mL). The organic phase was dried over anhydrous MgSO₄, filtered, and concentrated in vacuo to provide crude 13-4 (4 mg, yield: 3%) as a colorless oil. ESI-MS m/z: (M+H)⁺=275.3.

Step E: A solution of 13-4 (200 mg, 0.651 mmol) in AcOH/H₂O (1.1 mL) was heated to 100° C. and stirred for 30 h. After cooling to r.t., the reaction mixture was diluted with NaHCO₃ (20 mL) and extracted with EtOAc (200 mL). The combined organic layers were concentrated to give a residue, which was purified by silica gel chromatography (PE:EtOAc=5:1) to afford 13-5 (10 mg, yield: 3%) as a colorless oil. ESI-MS m/z: (M+H)⁺=217.2.

Step F: To a solution of 13-5 (50 mg, 0.23 mmol) in MeOH (1.5 mL) were added CH₆CINO (23.1 mg, 0.276 mmol) and CH₃COONa (22.7 mg, 0.276 mmol). The mixture was stirred at rt for 16 h, then concentrated in vacuo and the residue partitioned between DCM (200 mL) and H₂O (20 mL). The organic phase was dried over anhydrous MgSO₄, filtered, and concentrated in vacuo. The mixture was purified by silica gel chromatography to afford 13-6 (40 mg, yield: 70.51%) as a white solid. ESI-MS m/z: (M+H)⁺=232.2.

Step G: To a solution of 13-6 (37 mg, 0.15 mmol) in THF (2 mL) was added LiAlH₄ (17 mg, 0.45 mmol) at 65° C. and the resulting mixture was stirred for 5 h. Then, Na₂SO₄·10H₂O (39 mg, 0.12 mmol) was added at 0° C. The reaction mixture was stirred 0.5 h, then diluted with H₂O (50 mL) and extracted with EtOAc (200 mL). The combined organic layers were concentrated to give a residue. Crude 13-7 (150 mg, yield: 150%) was obtained as a colorless oil, which was used directly in the next step without further purification. ESI-MS m/z: (M+H)⁺=204.3.

Step H: A solution of 13-7 (150 mg, 0.735 mmol), DIPEA (190 mg, 1.47 mmol), and Boc₂O (468 mg, 1.47 mmol) in DCM (5 mL) was stirred for 16 h at room temperature. Water (10 mL) was added and the reaction mixture was stirred for 30 minutes. The mixture was extracted with chloroform. The organic layer was dried over sodium sulfate, filtered, and concentrated to dryness under reduced pressure to give a crude residue, which was purified by silica gel chromatography to provide tert-butyl (1-benzyl-2,2-dimethylpyrrolidin-3-yl) carbamate (4 mg, yield: 6.5%) as a colorless oil. ESI-MS m/z: (M+H)⁺=304.2.

Step I: To a solution of tert-butyl (1-benzyl-2,2-dimethylpyrrolidin-3-yl) carbamate (100 mg, 0.3287 mmol) in DMF (2 mL) was added NaH (39 mg, 1.625 mmol) at 0° C. The reaction mixture was stirred for 1 h at 0° C., then CH₃I (467 mg, 3.2901 mmol) was added and the resulting mixture was stirred at rt for 2 h. The mixture was diluted with EtOAc (100 mL) and washed with water (100 mL). The organic layer was separated, concentrated in vacuo, and the resulting crude residue was purified by silica gel chromatography to (PE:EtOAc=8:1) to give 13-8 (70 mg, yield: 66.9%) as a colorless oil. ESI-MS m/z: (M+H)⁺=318.4.

Step J: A mixture of 13-8 (70 mg, 0.22 mmol) and Pd/C (70 mg, 0.6578 mmol) in EtOAc (5 mL) was stirred under H₂ at rt for 3 h. Pd/C was filtered, then the organic solvent was concentrated to obtain 13-9 (40 mg, yield: 79.7%) as a colorless oil. ESI-MS m/z: (M+H)⁺=228.3.

Step K: To a solution of 13-9 (133 mg, 0.5829 mmol) in DMF (1.5 mL) were added 1-9 (150 mg, 0.2325 mmol), PyBop (242 mg, 0.4650 mmol) and DIEA (90 mg, 0.6963 mmol). The mixture was stirred at room temperature for 1 h, then diluted with EtOAc (150 mL) and washed with brine (100 mL). The organic layer was separated and concentrated in vacuo. The crude residue was purified by flash chromatography (DCM:MeOH=10:1) to give 13-10 (160 mg, yield: 80.4%) as a colorless oil. ESI-MS m/z: (M+H)+=856.4.

Step L: To a solution of 13-10 (120 mg, 0.1404 mmol) in DCM (1.5 mL) was added TFA (13.62 mg, 0.1404 mmol). The mixture was stirred at room temperature for 1 h, then basified with aqueous sodium bicarbonate. The mixture was diluted with (DCM:MeOH=10:1, 50 mL) and washed with brine (100 mL). The organic layer was separated and concentrated in vacuo. The crude residue was purified by flash chromatography (DCM:NH₃·MeOH=10:1) to give (4R)-2-amino-4-(6-chloro-4-(2,2-dimethyl-3-(methylamino) pyrrolidin-1-yl)-8-fluoro-2-(((2R,7aS)-2-fluorotetrahydro-1H-pyrrolizin-7a (5H)-yl) methoxy) quinazolin-7-yl)-7-fluorobenzo[b]thiophene-3-carbonitrile (80 mg, yield: 87.1%) as a yellow oil. ESI-MS m/z: (M+H)⁺=656.1.

Step M: To a solution of 3-7 (137 mg, 1.33 mmol) and pyridine (0.43 mL, 5.32 mmol) in MeCN (4.5 mL) was added BTC (356 mg, 1.20 mmol) at 0° C. The reaction mixture was stirred at 0° C. for 2 h, then added to a solution of (4R)-2-amino-4-(6-chloro-4-(2,2-dimethyl-3-(methylamino) pyrrolidin-1-yl)-8-fluoro-2-(((2R,7aS)-2-fluorotetrahydro-1H-pyrrolizin-7a(5H)-yl)methoxy)quinazolin-7-yl)-7-fluorobenzo[b]thiophene-3-carbonitrile (35 mg, 0.0532 mmol) in DME (0.7 mL) at rt. Pyridine (42.1 mg, 0.532 mmol) was added, then the mixture was concentrated and purified by prep-HPLC (FA) to give 198 (9.86 mg, yield: 23.5%) as a white solid. ESI-MS m/z: (M+H)⁺=785.4. ¹H NMR (400 MHZ, DMSO) δ 9.27 (s, 1H), 8.26-8.03 (m, 3H), 7.25-7.10 (m, 2H), 5.33 (d, J=52.0 Hz, 1H), 4.85-4.03 (m, 5H), 3.20-2.65 (m, 7H), 2.43-2.00 (m, 5H), 1.91-1.68 (m, 9H).

### Example 1n: Synthesis of (R)-2-amino-4-(6-chloro-8-fluoro-4-((1R,4R,7S)-2-(3-fluoro-1H-1,2,4-triazole-1-carbonyl)-1-isopropyl-7-methyl-2,6-diazaspiro[3.6]decan-6-yl)-2-(((2R,7aS)-2-fluorotetrahydro-1H-pyrrolizin-7a(5H)-yl)methoxy)quinazolin-7-yl)-7-fluorobenzo[b]thiophene-3-carbonitrile (212)

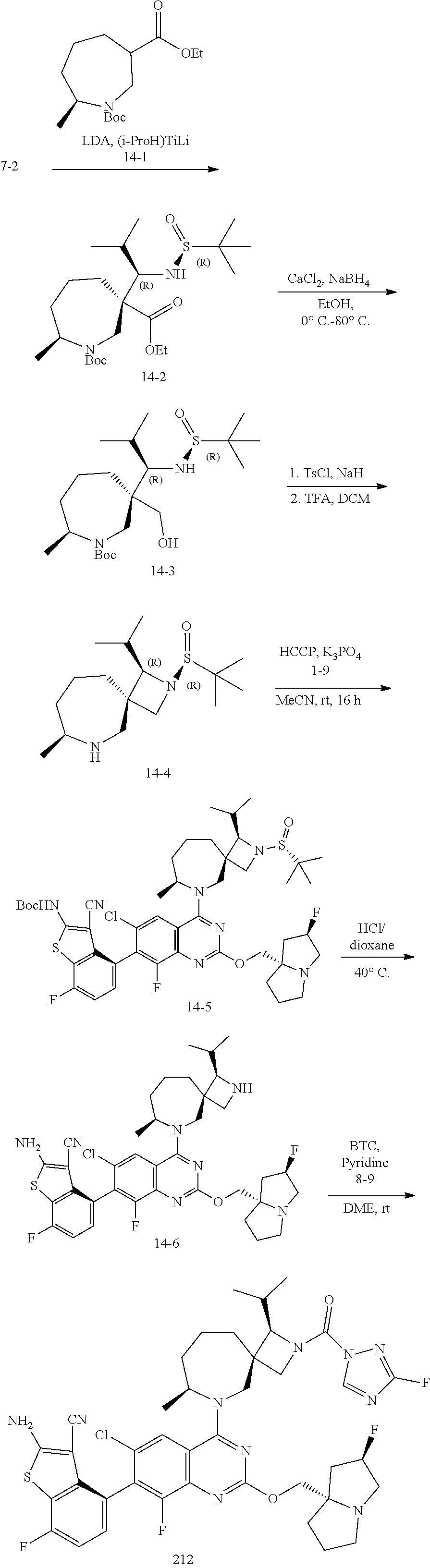

Step A: N-butyllithium (2.5 M in hexanes, 2.86 mL, 7.14 mmol) was added dropwise at 0° C. to a solution of diisopropylamine (0.73 g, 7.14 mmol) in dry THF (10 mL) and the solution was stirred at 0° C. for 0.5 h. The reaction mixture was then cooled to −78° C. followed by dropwise addition of a solution of 14-1 (3.26 g, 11.42 mmol). The resulting reaction mixture was stirred at −78° C. for 1 h, then a solution of (i-PrO)₃TiCl (8.6 mL, 8.6 mmol) was added dropwise and the reaction mixture was stirred for 1 h. A solution of 7-2 (1.4 g, 5.71 mmol) in THF (5 mL) was then added dropwise and the reaction mixture was stirred for an additional 1 h. The reaction mixture was diluted with NH₄Cl (aq) (70 mL) and extracted with EtOAc (70 mL×3). The organic layer was washed with brine, dried over sodium sulfate, filtered, and concentrated to dryness under reduced pressure to give a crude residue, which was purified by silica gel chromatography (PE/EtOAc=3/1) to give 14-2 as a colorless oil. (1.1 g, Yield: 42%). ESI-MS m/z: (M+H)⁺=461.3.

Step B: To a solution of 14-2 (1.1 g, 2.39 mmol) in dry EtOH (20 mL) was added CaCl₂) (1.06 g, 9.56 mmol, 4.0 eq) and NaBH₄ (0.723 g, 19.13 mmol, 8.0 eq) at 0° C. The mixture was stirred at 60° C. for 16 h, then aqueous citric acid (20 mL), water (50 mL) and EtOAc (50 mL) added. The organic layer was washed with brine, dried over sodium sulfate, filtered, and concentrated to dryness under reduced pressure to give a crude residue, which was purified by silica gel chromatography (PE:EtOAc=55:45) to give 14-3 (0.8 g, Yield: 83%) as a colorless oil. ESI-MS m/z: (M+H)⁺=419.3.

Step C: To a solution of 14-3 (690 mg, 1.65 mmol) and TsCl (470 mg, 2.48 mmol) in dry THF (10 mL) was added NaH (264 mg, 6.6 mmol) at 0° C. The mixture was stirred at 60° C. for 16 h, then diluted with NH₄Cl (aq) (20 mL) and extracted with EtOAc (3×50 mL). The organic layer was washed with brine, dried over sodium sulfate, filtered, and concentrated to dryness under reduced pressure to give a crude residue, which was purified by silica gel chromatography (PE/EtOAc=1/1) to give tert-butyl (1R,4R,7S)-2-((R)-tert-butylsulfinyl)-1-isopropyl-7-methyl-2,6-diazaspiro[3.6]decane-6-carboxylate as a colorless oil. (220 mg, yield: 33%). ESI-MS m/z: (M+H)⁺=401.2.

Step D: To a solution of tert-butyl (1R,4R,7S)-2-((R)-tert-butylsulfinyl)-1-isopropyl-7-methyl-2,6-diazaspiro[3.6]decane-6-carboxylate (230 mg, 0.58 mmol) in DCM (2 mL) was added TFA (0.4 mL) at 0° C. The mixture was stirred at 25° C. for 2 h, then diluted with aqueous NaHCO₃ (80 mL) and DCM:MeOH=10:1 (50×3 mL). The organic layer was washed with brine, dried over sodium sulfate, filtered, and concentrated to dryness under reduced pressure to give 14-4 (150 mg, yield: 87%) as a white solid. ESI-MS m/z: (M+H)⁺=301.2.

Step E: To a solution of 1-9 (265 mg, 0.41 mmol) in MeCN (2 mL) were added K₃PO₄ (218 mg, 1.03 mmol) and HCCP (142 mg, 0.41 mmol). The mixture was stirred for 1 h, then 14-4 (135 mg, 0.45 mmol) in MeCN (0.5 mL) was added and the mixture was stirred for 16 h. The solution was extracted with ethyl acetate (3×100 mL). The organic layer was washed with brine, dried over sodium sulfate, filtered, and concentrated to dryness under reduced pressure to give a crude residue, which was purified by silica gel chromatography (DCM/MeOH=15/1) to give 14-5 (170 mg, yield: 45%) as a yellow solid. Further purification by SFC gave 14-5-P1 (120 mg) and 14-5-P2 (30 mg). ESI-MS m/z: (M+H)⁺=928.1.

Step F: A solution of 14-5-P1 (120 mg, 0.13 mmol) in HCl/dioxane (4 mL) was stirred at 40° C. for 2 h. The solvent was removed under reduced pressure and the residue was diluted with aqueous NaHCO₃ (100 mL) and EtOAc (300 mL). The organic layer was washed with brine, dried over sodium sulfate, filtered, and concentrated to dryness under reduced pressure to give a crude residue, which was purified by silica gel chromatography (DCM/MeOH=10/1) to give 14-6 (60 mg, yield: 64%) as a yellow solid. ESI-MS m/z: (M+H)⁺=724.0.

Step G: To a solution of 8-9 (137 mg, 1.33 mmol) and pyridine (0.43 ml, 5.32 mmol) in MeCN (4.5 ml) was added BTC (356 mg, 1.20 mmol) at 0° C. The reaction mixture was then stirred at 0° C. for 2 h. To a solution of 14-6 (60 mg, 0.08 mmol) in DME (1 mL) was added pyridine (0.4 mL, 4.95 mmol) and above reaction mixture (0.8 mL) at rt. The mixture was concentrated and purified by prep-HPLC (FA) to give 212 (29.77 mg, yield: 43%) as a white solid. ESI-MS m/z: (M+H)⁺=837.2. ¹H NMR (400 MHZ, DMSO) δ 9.03 (s, 1H), 8.09 (s, 2H), 7.98 (s, 1H), 7.32-7.11 (m, 2H), 5.25 (d, J=54.0 Hz, 1H), 4.97 (d, J=14.4 Hz, 1H), 4.69-4.56 (m, 1H), 4.43-4.29 (m, 1H), 4.21-4.04 (m s, 2H), 3.99-3.86 (m, 2H), 3.51-3.41 (m, 1H), 3.29-3.27 (m, 1H), 3.13-3.00 (m, 2H), 2.90-2.76 (m, 1H), 2.42-2.34 (m, 1H), 2.19-1.96 (m, 4H), 1.89-1.71 (m, 5H), 1.67-1.54 (m, 3H), 1.20 (d, J=6.0 Hz, 3H), 1.13-1.03 (m, 6H).

### Example 1o: Synthesis of (R)-2-amino-4-(6-chloro-4-((1S,4R,7S)-1-(1-(difluoromethyl)cyclopropyl)-2-(3-fluoro-1H-1,2,4-triazole-1-carbonyl)-7-methyl-2,6-diazaspiro[3.5]nonan-6-yl)-8-fluoro-2-(((2R,7aS)-2-fluorotetrahydro-1H-pyrrolizin-7a(5H)-yl)methoxy)quinazolin- 7-yl)-7-fluorobenzo[b]thiophene-3-carbonitrile (194)

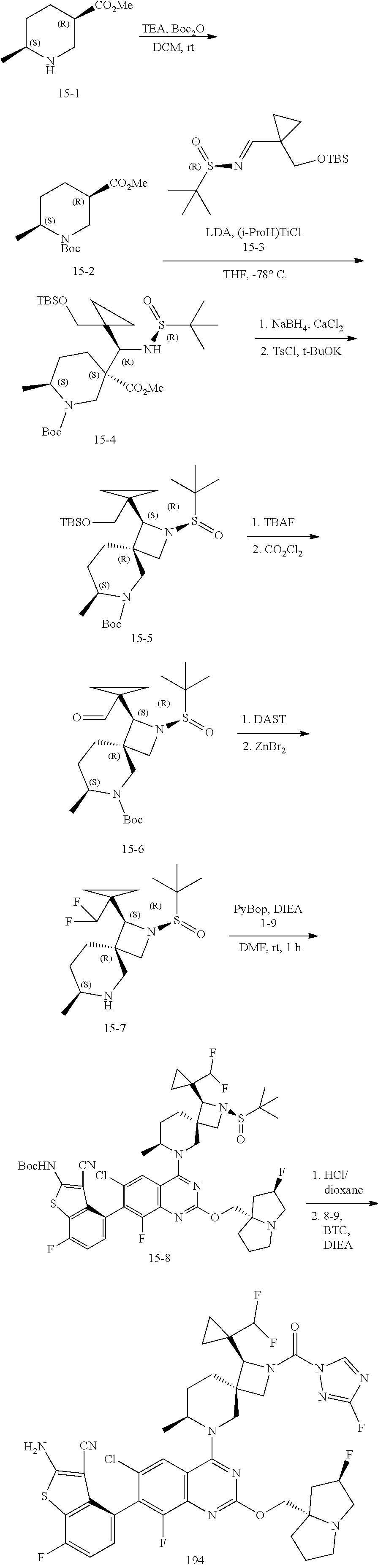

Step A: To a solution of 15-1 (3 g, 19.108 mmol) in DCM (30 mL) was added Boc₂O (6.25 g, 28.662 mmol) and TEA (5.79 g, 57.324 mmol) at 0° C. The mixture was stirred for 16 h at rt, then quenched with water (50 mL). The aqueous layer was extracted with EtOAc (2×50 mL). The organic layer was washed with brine, dried over sodium sulfate, filtered, and concentrated to dryness under reduced pressure to give a crude residue, which was purified by silica gel chromatography to afford 15-2 (4 g, 82% yield) as a yellow oil. ¹H NMR (400 MHZ, DMSO) δ 4.26-4.25 (m, 1H), 4.03-4.00 (m, 1H), 3.63-3.62 (m, 3H), 2.79-2.69 (m, 1H), 2.39-2.35 (m, 1H), 1.79-1.75 (m, 1H), 1.62-1.60 (m, 3H), 1.58-1.50 (m, 9H), 1.07-1.05 (m, 3H).

Step B: A solution of n-butyllithium (2.5 M in hexanes, 38.9 mL, 97.275 mmol) was added dropwise at 0° C. to a solution of diisopropylamine (9.82 g, 97.275 mmol) in THF (80 mL). The mixture was stirred at 0° C. for 0.5 h, then cooled to −78° C. followed by addition of a solution of 15-2 (10 g, 38.91 mmol) in THF (10 mL) dropwise. The resulting reaction mixture was stirred at −78° C. for 1.5 h. A solution of (i-PrO)₃TiCl (117 mL, 116.73 mmol) was added dropwise and the reaction was stirred for 1 h. A solution of 15-3 (12.33 g, 38.91 mmol) in THF (10 mL) was then added dropwise and the reaction was stirred for an additional 1 h at −78° C. The reaction mixture was diluted with aqueous NaH₃PO₄ (50 mL) and extracted with EtOAc (3×50 mL). The combined organic layers were concentrated to give a residue, which was purified by silica gel chromatography to afford 15-4 (16 g, 72% yield) as a yellow oil. ¹H NMR (400 MHZ, DMSO) δ 5.08-5.07 (m, 1H), 4.27-4.06 (m, 3H), 3.62-3.61 (m, 3H), 3.12-3.11 (m, 2H), 2.55-2.54 (m, 1H), 2.07-2.06 (m, 1H), 1.80-1.79 (m, 1H), 1.63-1.52 (m, 1H), 1.45-1.35 (m, 10H), 1.17-1.16 (m, 9H), 1.08-0.99 (m, 3H), 0.90-0.80 (m, 9H), 0.58-0.42 (m, 3H), 0.34-0.33 (m, 1H), 0.02-0.01 (m, 6H).

Step C: To a solution of 15-4 (500 mg, 0.871 mmol) in EtOH (6 mL) were added CaCl₂) (386 mg, 3.484 mmol) and NaBH₄ (265 mg, 6.968 mmol) at 0° C. The mixture was stirred for 16 h at rt, then quenched with citric acid (10 mL/1 g/mL). The aqueous layer was extracted with EtOAc (2×50 mL). The organic layer was washed with brine, dried over sodium sulfate, filtered, and concentrated to dryness under reduced pressure to give a crude residue, which was purified by flash chromatography to afford tert-butyl (2S,5S)-5-((R)-(1-(((tert-butyldimethylsilyl)oxy)methyl)cyclopropyl)(((R)-tert-butylsulfinyl)amino)methyl)-5-(hydroxymethyl)-2-methylpiperidine-1-carboxylate (300 mg, 63% yield) as a white solid. ¹H NMR (400 MHZ, DMSO) δ 5.52-5.51 (m, 1H), 4.95-4.94 (m, 1H), 4.18-4.17 (m, 1H), 3.90-3.61 (m, 4H), 3.43-3.42 (m, 1H), 2.87-2.86 (m, 1H), 2.42-2.41 (m, 1H), 1.71-1.70 (m, 2H), 1.49-1.46 (m, 1H), 1.38-1.35 (m, 9H), 1.32-1.23 (m, 1H), 1.15-1.14 (m, 9H), 1.07-1.06 (m, 3H), 0.86-0.85 (m, 9H), 0.67-0.65 (m, 1H), 0.58-0.44 (m, 2H), 0.35-0.24 (m, 1H), 0.03-0.02 (m, 6H).

Step D: To a solution of tert-butyl (2S,5S)-5-((R)-(1-(((tert-butyldimethylsilyl)oxy)methyl)cyclopropyl)(((R)-tert-butylsulfinyl)amino)methyl)-5-(hydroxymethyl)-2-methylpiperidine-1-carboxylate (1.5 g, 2.747 mmol) in THF (75 mL) were added TsCl (1.04 g, 5.494 mmol) and potassium tert-butoxide (1.538 g, 13.735 mmol) at 0° C. The resulting mixture was stirred for 16 h at rt, then quenched with water (10 mL) and the aqueous layer extracted with EtOAc (3×40 mL). The organic layer was washed with brine, dried over sodium sulfate, filtered, and concentrated to dryness under reduced pressure to give a crude residue, which was purified by flash chromatography to afford 15-5 (800 mg, 55% yield) as a yellow oil. ¹H NMR (400 MHZ, DMSO) δ 4.27-4.26 (m, 2H), 3.89-3.88 (m, 1H), 3.67-3.66 (m, 2H), 3.27-3.26 (m, 1H), 3.06-3.05 (m, 1H), 2.78-2.76 (m, 1H), 1.71-1.70 (m, 1H), 1.51-1.49 (m, 2H), 1.39-1.35 (m, 9H), 1.09-1.02 (m, 12H), 0.92-0.82 (m, 10H), 0.56-0.55 (m, 1H), 0.44-0.43 (m, 1H), 0.27-0.26 (m, 1H), 0.04-0.02 (m, 6H).

Step E: To a solution of 15-5 (3.7 g, 7.582 mmol) in THF (25 mL) was added TBAF (15.16 mL, 15.16 mmol). The mixture was stirred for 4 h at rt, then quenched by the addition of saturated aqueous NH₄Cl (30 mL). The aqueous layer was extracted with EtOAc (2×30 mL). The organic layer was washed with brine, dried over sodium sulfate, filtered, and concentrated to dryness under reduced pressure to give a crude residue, which was purified by flash chromatography to afford tert-butyl (1S,4R,7S)-2-((R)-tert-butylsulfinyl)-1-(1-(hydroxymethyl)cyclopropyl)-7-methyl-2,6-diazaspiro[3.5]nonane-6-carboxylate (2 g, 71% yield) as a yellow oil. ¹H NMR (400 MHZ, DMSO) δ 4.03-4.01 (m, 2H), 3.41-3.39 (m, 1H), 3.05-3.03 (m, 5H), 2.55-2.53 (m, 1H), 1.63-1.60 (m, 1H), 1.30-1.11 (m, 12H), 0.84-0.83 (m, 12H), 0.60-0.59 (m, 1H), 0.34-0.33 (m, 1H), 0.16-0.15 (m, 1H), 0.03-0.02 (m, 1H).

Step F: To a solution of oxalyl chloride (480 mg, 3.816 mmol) in DCM (10 mL) was added DMSO (595 mg, 7.632 mmol) at −65° C. and a the resulting mixture was stirred for 30 min. Tert-butyl (1S,4R,7S)-2-((R)-tert-butylsulfinyl)-1-(1-(hydroxymethyl)cyclopropyl)-7-methyl-2,6-diazaspiro[3.5]nonane-6-carboxylate (790 mg, 1.908 mmol) in DCM (5 mL) was added and the mixture was stirred for 1 h at −65° C. Et₃N (1.1 g, 11.448 mmol) was added and stirred for 20 min at −65° C. The mixture was stirred for 30 min at 0° C., then quenched with water (5 mL). THF:DCM=5:1 (20 mL) added, then the mixture was stirred for 10 min and filtered. The filtrate was concentrated under vacuum and purified by silica gel chromatography to afford 15-6 (600 mg, 80% yield) as a yellow oil. ¹H NMR (400 MHZ, DMSO) δ 8.67 (s, 1H), 4.26-4.05 (m, 3H), 3.72-3.69 (m, 1H), 2.90-2.89 (m, 1H), 2.67-2.65 (m, 1H), 2.03-2.01 (m, 1H), 1.60-1.38 (m, 14H), 1.29-1.21 (m, 1H), 1.11-0.98 (m, 13H).

Step G: To a solution of 15-6 (700 mg, 1.699 mmol) in DCM (20 mL) was added DAST (1.37 g, 8.495 mmol). The mixture was stirred for 16 h at rt, then quenched by the addition of saturated aqueous NaHCO₃ (20 mL). The aqueous layer was extracted with EtOAc (2×30 mL). The organic layer was washed with brine, dried over sodium sulfate, filtered, and concentrated to dryness under reduced pressure to give a crude residue, which was purified by flash chromatography to afford tert-butyl (1S,4R,7S)-2-((R)-tert-butylsulfinyl)-1-(1-(difluoromethyl)cyclopropyl)-7-methyl-2,6-diazaspiro[3.5]nonane-6-carboxylate (600 mg, 81% yield) as a yellow oil. ¹H NMR (400 MHZ, DMSO) δ 5.77-5.76 (m, 1H), 4.23-4.22 (m, 2H), 3.98-3.96 (m, 1H), 3.68-3.65 (m, 1H), 3.04-3.03 (m, 1H), 2.81-2.79 (m, 1H), 1.76-1.75 (m, 1H), 1.56-1.37 (m, 12H), 1.11-0.96 (m, 14H), 0.64-0.62 (m, 2H).

Step H: To a solution of tert-butyl (1S,4R,7S)-2-((R)-tert-butylsulfinyl)-1-(1-(difluoromethyl)cyclopropyl)-7-methyl-2,6-diazaspiro[3.5]nonane-6-carboxylate (170 mg, 0.392 mmol) in DCM (15 mL) was added ZnBr₂ (352 mg, 1.567 mmol) and the reaction mixture was stirred for 16 h at 50° C. The reaction mixture was quenched by the addition of saturated aqueous NaHCO₃ (15 mL) and the aqueous layer was extracted with EtOAc (3×30 mL). The organic layer was washed with brine, dried over sodium sulfate, filtered, and concentrated to dryness under reduced pressure to give a crude residue, which was purified by flash chromatography to afford 15-7 (120 mg, 92% yield) as a yellow oil. ESI-MS m/z: (M+H)⁺=335.2.

Step I: To a solution of 15-7 (60 mg, 0.179 mmol) in DMF (2 mL) was added 1-9 (100 mg, 0.155 mmol), PyBop (242 mg, 0.465 mmol) and DIPEA (100 mg, 0.775 mmol). The reaction mixture was stirred for 1 h at rt, then quenched with water (5 mL). The aqueous layer was extracted with EtOAc (2×20 mL). The organic layer was washed with brine, dried over sodium sulfate, filtered, and concentrated to dryness under reduced pressure to give a crude residue, which was purified by flash chromatography to afford 15-8 (110 mg, 74% yield) as a yellow oil. ESI-MS m/z: (M+H)⁺=962.4.

Step J: A mixture of 15-8 (150 mg, 0.156 mmol) in HCl in dioxane (5 mL) was stirred for 1 h at 40° C. The reaction mixture was quenched NaHCO₃ (20 mL) and the aqueous layer was extracted with EtOAc (2×30 mL). The organic layer was washed with brine, dried over sodium sulfate, filtered, and concentrated to dryness under reduced pressure to give a crude residue, which was purified by flash chromatography to afford (R)-2-amino-4-(6-chloro-4-((1S,4S,7S)-1-(1-(difluoromethyl)cyclopropyl)-7-methyl-2,6-diazaspiro[3.5]nonan-6-yl)-8-fluoro-2-(((2R,7aS)-2-fluorotetrahydro-1H-pyrrolizin-7a (5H)-yl) methoxy) quinazolin-7-yl)-7-fluorobenzo[b]thiophene-3-carbonitrile (80 mg, 67% yield) as a yellow solid. ESI-MS m/z: (M+H)⁺=758.6.

Step K: To a solution of 8-9 (137 mg, 1.15 mmol) and pyridine (0.37 mL, 4.6 mmol) in MeCN (4.5 ml) was added BTC (307 mg, 1.03 mmol) at 0° C. The reaction mixture was stirred at 0° C. for 2 h. To a solution of (R)-2-amino-4-(6-chloro-4-((1S,4S,7S)-1-(1-(difluoromethyl)cyclopropyl)-7-methyl-2,6-diazaspiro[3.5]nonan-6-yl)-8-fluoro-2-(((2R,7aS)-2-fluorotetrahydro-1H-pyrrolizin-7a(5H)-yl)methoxy)quinazolin-7-yl)-7-fluorobenzo[b]thiophene-3-carbonitrile (60 mg, 0.084 mmol) in DME (0.7 mL) was added pyridine (0.37 mL, 4.6 mmol) and the above reaction mixture (0.6 mL) at 30° C. The mixture was poured into water (10 mL) and extracted with ethyl acetate (3×10 mL). The organic layer was washed with brine, dried over sodium sulfate, filtered, and concentrated to dryness under reduced pressure to give a crude residue, which was purified by prep-HPLC (FA) to give 194 (19 mg, yield: 28%) as a white solid. ESI-MS m/z: (M+H)⁺=871.2. ¹H NMR (400 MHZ, DMSO) δ 9.07-9.06 (m, 1H), 8.11-8.09 (m, 2H), 7.81-7.79 (m, 1H), 7.32-7.11 (m, 2H), 5.99-5.98 (m, 1H), 5.27-5.26 (m, 1H), 4.92-4.65 (m, 2H), 4.17-4.16 (m, 4H), 3.61-3.59 (m, 1H), 3.14-2.77 (m, 4H), 2.21-1.69 (m, 9H), 1.55-1.53 (m, 1H), 1.35-1.34 (m, 3H), 1.22-1.02 (m, 2H), 0.75-0.74 (m, 2H).

### Example 2: Ras Sequences

| Human K-Ras Wildtype sequence | |
|---|---|
| (SEQ ID NO. 1) | |
| 1 | MTEYKLVVVG AGGVGKSALT IQLIQNHFVD EYDPTIEDSY RKQVVIDGET |
| 51 | CLLDILDTAG QEEYSAMRDQ YMRTGEGFLC VFAINNTKSF EDIHHYREQI |
| 101 | KRVKDSEDVP MVLVGNKCDL PSRTVDTKQA QDLARSYGIP FIETSAKTRQ |
| 151 | GVDDAFYTLV REIRKHKEKM SKDGKKKKKK SKTKCVIM |
| Human K-Ras G12D | |
| (SEQ ID NO. 2) | |
| 1 | MTEYKLVVVG ADGVGKSALT IQLIQNHFVD EYDPTIEDSY RKQVVIDGET |
| 51 | CLLDILDTAG QEEYSAMRDQ YMRTGEGFLC VFAINNTKSF EDIHHYREQI |
| 101 | KRVKDSEDVP MVLVGNKCDL PSRTVDTKQA QDLARSYGIP FIETSAKTRQ |
| 151 | GVDDAFYTLV REIRKHKEKM SKDGKKKKKK SKTKCVIM |
| Human K-Ras G12V | |
| (SEQ ID NO. 3) | |
| 1 | MTEYKLVVVG AVGVGKSALT IQLIQNHFVD EYDPTIEDSY RKQVVIDGET |
| 51 | CLLDILDTAG QEEYSAMRDQ YMRTGEGFLC VFAINNTKSF EDIHHYREQI |
| 101 | KRVKDSEDVP MVLVGNKCDL PSRTVDTKQA QDLARSYGIP FIETSAKTRQ |
| 151 | GVDDAFYTLV REIRKHKEKM SKDGKKKKKK SKTKCVIM |
| Human K-Ras G12S (SEQ ID NO. 4): | |
| 1 | MTEYKLVVVG ASGVGKSALT IQLIQNHFVD EYDPTIEDSY RKQVVIDGET |
| 51 | CLLDILDTAG QEEYSAMRDQ YMRTGEGFLC VFAINNTKSF EDIHHYREQI |
| 101 | KRVKDSEDVP MVLVGNKCDL PSRTVDTKQA QDLARSYGIP FIETSAKTRQ |
| 151 | GVDDAFYTLV REIRKHKEKM SKDGKKKKKK SKTKCVIM |
| Human N-Ras wildtype | |
| (SEQ ID NO. 5) | |
| 1 | MTEYKLVVVG AGGVGKSALT IQLIQNHFVD EYDPTIEDSY RKQVVIDGET |
| 51 | CLLDILDTAG QEEYSAMRDQ YMRTGEGFLC VFAINNSKSF ADINLYREQI |
| 101 | KRVKDSDDVP MVLVGNKCDL PTRTVDTKQA HELAKSYGIP FIETSAKTRQ |
| 151 | GVEDAFYTLV REIRQYRMKK LNSSDDGTQG CMGLPCVVM |
| H-Ras G12D | |
| (SEQ ID NO. 6) | |
| 1 | MTEYKLVVVG ADGVGKSALT IQLIQNHFVD EYDPTIEDSY RKQVVIDGET |
| 51 | CLLDILDTAG QEEYSAMRDQ YMRTGEGFLC VFAINNTKSF EDIHQYREQI |
| 101 | KRVKDSDDVP MVLVGNKCDL AARTVESRQA QDLARSYGIP YIETSAKTRQ |
| 151 | GVEDAFYTLV REIRQHKLRK LNPPDESGPG CMSCKCVLS |
| H-Ras wildtype | |
| (SEQ ID NO. 7) | |
| 1 | MTEYKLVVVG AGGVGKSALT IQLIQNHFVD EYDPTIEDSY RKQVVIDGET |
| 51 | CLLDILDTAG QEEYSAMRDQ YMRTGEGFLC VFAINNTKSF EDIHQYREQI |
| 101 | KRVKDSDDVP MVLVGNKCDL AARTVESRQA QDLARSYGIP YIETSAKTRQ |
| 151 | GVEDAFYTLV REIRQHKLRK LNPPDESGPG CMSCKCVLS |
| Human N-Ras G12D | |
| (SEQ ID NO. 8) | |
| 1 | MTEYKLVVVG ADGVGKSALT IQLIQNHFVD EYDPTIEDSY RKQVVIDGET |
| 51 | CLLDILDTAG QEEYSAMRDQ YMRTGEGFLC VFAINNSKSF ADINLYREQI |
| 101 | KRVKDSDDVP MVLVGNKCDL PTRTVDTKQA HELAKSYGIP FIETSAKTRQ |
| 151 | GVEDAFYTLV REIRQYRMKK LNSSDDGTQG CMGLPCVVM |

### Example 3: Protein Expression

DNA expression constructs encoding one or more protein sequences of interest (e.g., Kras fragments thereof, mutant variants thereof, etc.) and its corresponding DNA sequences are optimized for expression in E. coli and synthesized by, for example, the GeneArt Technology at Life Technologies. In some cases, the protein sequences of interest are fused with a tag (e.g., glutathione S-transferase (GST), histidine (His), or any other affinity tags) to facilitate recombinant expression and purification of the protein of interest. Such tag can be cleaved subsequent to purification. Alternatively, such tag may remain intact to the protein of interest and may not interfere with activities (e.g., target binding and/or phosphorylation) of the protein of interest

A resulting expression construct is additionally encoded with (i) att-site sequences at the 5′ and 3′ ends for subcloning into various destination vectors using, for example, the Gateway Technology, as well as (ii) a Tobacco Etch Virus (TEV) protease site for proteolytic cleavage of one or more tag sequences. The applied destination vectors can be a pET vector series from Novagen (e.g., with ampicillin resistance gene), which provides an N-terminal fusion of a GST-tag to the integrated gene of interest and/or a pET vector series (e.g., with ampicillin resistance gene), which provides an N-terminal fusion of a HIS-tag to the integrated gene. To generate the final expression vectors, the expression construct of the protein of interest is cloned into any of the applied destination vectors. The expression vectors are transformed into an E. coli strain, e.g., BL21 (DE3). Cultivation of the transformed strains for expression is performed in a 10 L or 1 L fermenter. The cultures are grown, for example, in Terrific Broth media (MP Biomedicals, Kat. #1 13045032) with 200 μg/mL ampicillin at a temperature of 37° C. to a density of 0.6 (OD600), shifted to a temperature of ˜27° C. (for K-Ras expression vectors) induced for expression with 100 mM IPTG, and further cultivated for 24 hours. After cultivation, the transformed E. coli cells are harvested by centrifugation and the resulting pellet is suspended in a lysis buffer, as provided below, and lysed by passing three-times through a high-pressure device. The lysate is centrifuged (49000 g, 45 min, 4° C.) and the supernatant is used for further purification.

### Example 4: Ras Protein Purification

A Ras (e.g., K-Ras wildtype or a mutant such as K-Ras G12S, K-Ras G12D, K-Ras G12V or K-Ras G12C) construct or a variant thereof is tagged with GST. E. coli culture from a 10L fermenter is lysed in lysis buffer (50 mM Tris HCl 7.5, 500 mM NaCl, 1 mM DTT, 0.5% CHAPS, Complete Protease Inhibitor Cocktail-(Roche)). As a first chromatography step, the centrifuged lysate is incubated with 50 mL Glutathione Agarose 4B (Macherey-Nagel; 745500.100) in a spinner flask (16 h, 10° C.). The Glutathione Agarose 4B loaded with protein is transferred to a chromatography column connected to a chromatography system, e.g., an Akta chromatography system. The column is washed with wash buffer (50 mM Tris HCl 7.5, 500 mM NaCl, 1 mM DTT) and the bound protein is eluted with elution buffer (50 mM Tris HCl 7.5, 500 mM NaCl, 1 mM DTT, 15 mM Glutathione). The main fractions of the elution peak (monitored by OD280) are pooled. For further purification by size-exclusion chromatography, the above eluate volume is applied to a column Superdex 200 HR prep grade (GE Healthcare) and the resulting peak fractions of the eluted fusion protein is collected. Native mass spectrometry analyses of the final purified protein construct can be performed to assess its homogeneous load with GDP.

### Example 5: HTRF (Homogenous Time-Resolved Fluorescence) Resonance Energy Transfer Assay

The ability of a compound of the present disclosure to reduce Ras signaling output can be demonstrated by an HTRF assay. This assay can be also used to assess a selective inhibition or reduction of signaling output of a mutant Ras protein relative to a wildtype, or relative to a different mutant Ras protein. For example, the equilibrium interaction of wildtype Kras or K-Ras mutant (e.g., wildtype or a mutant thereof) with SOS1 (e.g., hSOS1) can be assessed as a proxy or an indication for the ability of a subject compound to bind and inhibit Ras protein. The HTRF assay detects from (i) a fluorescence resonance energy transfer (FRET) donor (e.g., antiGST-Europium) that is bound to GST-tagged K-Ras mutant to (ii) a FRET acceptor (e.g., anti-6His-XL665) bound to a His-tagged hSOS1.

The assay buffer can contain ˜5 mM HEPES pH 7.4, ˜150 mM NaCl, ˜ 1 mM DTT, 0.05% BSA and 0.0025% (v/v) Igepal. A Ras working solution is prepared in an assay buffer containing typically a suitable amount of the protein construct (e.g., GST-tagged K-Ras mutant) and the FRET donor (e.g., antiGST-Eu (K) from Cisbio, France). A SOS1 working solution is prepared in an assay buffer containing suitable amount of the protein construct (e.g., His-hSOS1) and the FRET acceptor (e.g., anti-6His-XL665 from Cisbio, France). A suitable amount of the protein construct will depend on the range of activity or range of IC50 values being detected or under investigation. For detecting an IC50 within a range of 500 nM, the protein constructs of the same range of molarity can be utilized. An inhibitor control solution is prepared in an assay buffer containing a comparable amount of the FRET acceptor without the SOS1 protein.

A fixed volume of DMSO with or without test compound is transferred into a 384-well plate. Ras working solution is added to all wells of the test plate. SOS1 working solution is added to all wells except for those that are subsequently filled with inhibitor control solution. Upon incubation for about 10 minutes or longer, the fluorescence is measured with a M1000Pro plate reader (Tecan) using HTRF detection (excitation 337 nm, emission 1:620 nm, emission 2:665 nm). Compounds are tested in duplicate at different concentrations (for example, 10 μM, 2.5 μM, 0.63 μM, 0.16 μM, 0.04 μM, 0.01 μM test compound). The ratiometric data (i.e., emission 2 divided by emission 1) is used to calculate IC50 values against Ras using GraphPad Prism (GraphPad software). Signaling output measured in terms of IC50 values can be obtained and a ratio of IC50 against one mutant relative to another mutant can be calculated. For instance, a selective reduction of K-Ras G12S signaling output can be evidenced by a ratio greater than one. In particular, a selective reduction of K-Ras G12S signaling relative to K-Ras WT signaling is evidenced if the ratio of IC50 (against K-Ras WT) to IC50 (against K-Ras G12S) is greater than 1. In some embodiments, one or more subject compounds disclosed herein are expected to exhibit selective inhibition of a Ras mutant (e.g., G12C, G12S, or G13C) over WT by at least 1-fold, and in some instances greater than 2-, 3—, 4- or 5-fold. In some embodiments, subject compounds are expected to exhibit an IC50 against KRas mutants (e.g., G12C or G12S) less than 500 nM, such as less than 100 nM, 50 nM, or even less.

### Example 6: GTPase Activity Assay

The ability of a compound of the present disclosure to inhibit Ras protein signaling can be demonstrated by a reduced GTPase activity. This assay can also be used to assess selective inhibition of a mutant Ras protein relative to a wildtype or different mutant Ras protein. For instance, the assay can be used to establish a subject compound's ability to selectively inhibit Kras G12S relative to wildtype, Kras G12S relative to Kras G12V, Kras G12S relative to Kras G12D, Kras G12C relative to Kras G12D, or Kras G12C relative to Kras G12V or wildtype. In particular, intrinsic and GTPase-activating protein (GAP)-stimulated GTPase activity for a K-Ras construct or a mutant thereof can be measured using EnzCheck phosphate assay system (Life Technologies). For example, K-Ras WT, K-Ras D154Q mutant, K-Ras G12D mutant, K-Ras G12S mutant, and K-Ras G12D/D154Q mutant proteins (2.5 mg/mL) in buffer (20 mmol/L Tris, pH 8.0, 50 mM NaCl) are loaded with GTP at room temperature for 2 hours by exposing to exchange buffer containing EDTA. Proteins are buffer exchanged to assay buffer (30 mM Tris, pH 7.5, 1 mM DTT) and the concentration is adjusted to 2 mg/mL. GTP loading is verified by back extraction of nucleotide using 6M urea and evaluation of nucleotide peaks by HPLC using an ion-exchange column. The assay is performed in a clear 384-well plate (Costar) by combining GTP-loaded K-Ras proteins (50 mM final) with 2-amino-6-mercapto-7-methylpurine ribonucleoside (MESG) (200 mM final), and purine nucleotide phosphorylase (5 U/mL final). GTP hydrolysis is initiated by the addition of MgCl₂ at a working concentration of 40 mM. For GAP stimulation, Ras p21 protein activator 1 (P120GAP) can be included at 50 mM. Absorbance at 360 nm can be measured every 8 to 15 s for 1,000 s at 20° C. Samples are tested with or without a subject compound disclosed herein to assess the ability of each compound to inhibit signaling of a given Ras protein (e.g., a given mutant Kras) of interest.

### Example 7: Nucleotide Exchange Assay

The ability of a compound of the present disclosure to inhibit Ras protein signaling can be demonstrated by reduced nucleotide exchange activity. This assay can be also used to assess selective inhibition of a mutant Ras protein relative to a wildtype or a different mutant Ras protein. For example, 250 nM or 500 nM GDP-loaded K-Ras protein (e.g., wildtype or a mutant thereof including those mentioned in Example 4) is incubated with different concentrations of compounds (for example ˜60 μM, ˜20 μM, ˜6.7 μM, ˜2.2 μM, ˜0.7 μM, or ˜0.2 μM subject compound). A control reaction without subject compound is also included. SOS1 (catalytic domain) protein is added to the K-Ras protein solution. The nucleotide exchange reaction is initiated by adding fluorescent labelled GDP (Guanosine 5′-Diphosphate, BODIPY™ FL 2′-(or-3′)-O—(N-(2-Aminoethyl) Urethane) to a final concentration of 0.36 μM. Fluorescence is measured every 30 s for 70 minutes at 490 nm/515 nm (excitation/emission) in a M1000Pro plate reader (Tecan). Data is exported and analyzed to calculate an IC50 using GraphPad Prism (GraphPad Software). Sample(s) can be tested with or without a subject compound disclosed herein to assess the ability of the compound to inhibit K-Ras signaling or its IC50 against a given Ras protein (e.g., a given mutant K-Ras) of interest.

### Example 8: Testing for Modification of Ras Protein Via Covalent Binding

Test compounds are prepared as 10 mM stock solutions in DMSO (Fisher cat #BP231-100). KRAS protein (His-tagged GDP-loaded wildtype 1-169, His-tagged GDP-loaded G12S 1-169 or His-tagged GDP-loaded G12D 1-169) is diluted to ˜2 μM in appropriate buffer (e.g., a Hepes buffer at physiological conditions). For testing KRAS modification, compounds are diluted to 50× final test concentration in DMSO in 96-well storage plates. 2 μL of the diluted 50× compounds are added to appropriate wells in the PCR plate (Fisher cat #AB-0800). ˜49 μL of the stock protein solution is added to each well of the 96-well PCR plate. Reactions are mixed carefully. The plate is sealed well with aluminum plate seal and stored in a drawer at room temperature for 24 hrs. 5 μL of 2%-formic acid (Fisher cat #A117-50) in MilliQ H₂O is then added to each well followed by mixing with a pipette. The plate is then resealed with aluminum seal and stored until mass spectrometry analysis.

The extent of covalent modification of KRAS proteins was determined by liquid chromatography electrospray mass spectrometry analysis of the intact proteins on a Thermo Q-Exactive Plus mass spectrometer. 20 μL of sample was injected onto a bioZen 3.6 μm Intact C4 column (Phenomenex cat #00B-4767-AN) placed in a column oven set to 40° C. and separated using a suitable LC gradient from ˜20% to ˜60% solvent B. Solvent A was 0.1% formic acid and solvent B was 0.1% formic acid in acetonitrile. HESI source settings were set to 40, 5 and 1 for the sheath, auxiliary and sweep gas flow, respectively. The spray voltage was 4 kV, and the capillary temperature was 320° C. S-lens RF level was 50 and auxiliary gas heater temperature was set to 200° C. The mass spectrometry was acquired using a scan range from 650 to 1750 m/z using positive polarity at a mass resolution of 70,000, AGC target of 1e6 ions and maximum injection time of 250 ms. The recorded protein mass spectrum was deconvoluted from the raw data file using Protein Deconvolution v4.0 (Thermo). The protein mass and adduct masses were exported with their peak intensities. The peak intensities for the unmodified and modified protein were used to calculate the percent covalent modification of the KRAS protein based on the following equation: % KRAS protein modification=((KRAS-compound)/(KRAS)+(KRAS-Compound))*100.

Table 2 shows the percent modification at 24 hours of selected compounds against K-Ras G12S using the assay described above. Compound numbers correspond to the numbers and structures provided in Table 1 and Example 1.

TABLE 2

| | 1 to 20% | >20% |
|---|---|---|
| % G12S | 122, 155, 156, 183, | 101, 102, 103, 104, 105, 106, 109, 110, 111, 112, 113, 114, |
| Modification at 24 h | 188, 197, 201, 211, | 115, 116, 117, 118, 119, 120, 123, 124, 125, 126, 127, 128, |
| | 212, 214, 215, 221, | 129, 130, 131, 132, 133, 134, 135, 136, 137, 138, 139, 140, |
| | 222, 225, 233 | 141, 142, 143, 144, 145, 146, 147, 148, 149, 150, 151, 152, |
| | | 153, 154, 157, 158, 159, 160, 161, 162, 163, 164, 165, 166, |
| | | 167, 168, 169, 170, 171, 172, 174, 175, 176, 177, 178, 179, |
| | | 180, 181, 182, 184, 185, 186, 190, 192, 193, 194, 195, 196, |
| | | 198, 199, 200, 202, 203, 204, 205, 206, 207, 208, 209, 210, |
| | | 213, 216, 217, 218, 220, 223, 226, 227, 228, 229, 230, 231, |
| | | 232, 234, 235, 236, 237 |

The disclosed or exemplified warheads contained in the compounds disclosed herein exhibit superior properties as compared to conventional warheads including acrylamides, chlorofluoracetamides, epoxides, and nitriles. For example, these conventional warheads substantially lack the ability to engage with the serine residue, particularly at position 12 of K-Ras G12S mutant, even though these warheads may engage with a cysteine residue at position 12 of K-Ras. The present disclosure provides warheads that are particularly suited for reacting with the serine residue at position 12 of K-Ras G12S mutant as a solution to this problem. In addition, certain compounds disclosed herein are capable of engaging both serine and cysteine residues at position 12 of K-Ras G12S and K-Ras G12C, respectively.

### Example 9: Ras Cellular Assay

The ability of a compound of the present disclosure to inhibit Ras protein signaling can be demonstrated by inhibiting growth of a given Kras mutant cell line. For example, this assay can be also used to assess selective growth inhibition of a mutant Ras protein relative to a wildtype or different mutant Ras protein.

a. Growth of Cells with K-Ras G12C Mutation

MIA PaCa-2 (ATCC CRL-1420) and NCI-H1792 (ATCC CRL-5895) cell lines comprise a G12C mutation and can be used to assess Ras cellular signaling in vitro, e.g., in response to an inhibitor compound of the present disclosure. This cellular assay can also be used to discern selective inhibition of a subject compound against certain types of Kras mutants, e.g., more potent inhibition against Kras G12C relative to Kras G12D mutant, by comparing inhibition of MIA PaCa-2 (G12C driven tumor cell line) to inhibition of GP2d (G12D driven tumor cell line). MIA PaCa-2 culture medium is prepared with DMEM/Ham's F12 (e.g., with stable Glutamine, 10% FCS, and 2.5% Horse Serum. NCI-H1792 culture medium is prepared with RPMI 1640 (e.g., with stable Glutamine) and 10% FCS.

On a first day (e.g., Day 1), Softagar (Select Agar, Invitrogen, 3% in ddH₂O autoclaved) is boiled and tempered at 48° C. Appropriate culture medium (i.e., medium) is tempered to 37° C. Agar (3%) is diluted 1:5 in medium (=0.6%) and plated into 96 well plates (Corning, #3904), then incubated at room temperature for agar solidification. A 3% agar is diluted to 0.25% in medium (1:12 dilution) and tempered at 42° C. Cells are trypsinized, counted, and tempered at 37° C. The cells (e.g., MIA PaCa-2 at about 125-150 cells, NCI-H1792 at about 1000 cells) are resuspended in 100 mL 0.25% Agar and plated, followed by incubation at room temperature for agar solidification. The wells are overlaid with 50 mL of the medium. Sister wells in a separate plate are plated for time zero determination. All plates are incubated overnight at 37° C. and 5% CO₂.

On a second day (e.g., Day 2), time zero values are measured. A 40 mL volume of Cell Titer 96 Aqueous Solution (Promega) is added to each well and incubated in the dark at 37° C. and 5% CO₂. Absorption can be measured at 490 nm and reference wavelength 660 nm. DMSO-prediluted test compounds are added to wells of interest, e.g., with HP Dispenser, to one or more desired concentrations (e.g., a final DMSO concentration of 0.3%).

On a tenth day (e.g., Day 10), absorption by wells treated with the test compounds and control wells are measured with, for example, Cell Titer 96 AQueous and analyzed in comparison to the time zero measurements. The IC50 values are determined using the four parameter fit. The resulting IC50 value is a measurement of the ability of the test compound to reduce cell growth of Ras-driven cells (e.g., tumor cell lines) in vitro and/or in vivo. One or more compounds disclosed herein is expected to exhibit an IC50 value less than 5 μM, 1 μM, 100 nM, or even less, against one or more KRas G12C cell line (including MIA PaCa-2 and NCI-H1792).

b. Growth of Cells with K-Ras G12D Mutation

ASPC-1 (ATCC CRL-1682), Panc-10.05 (ATCC CRL-2547), A427, and GP2d cell lines, or any other cell lines comprising a G12D mutation, can be used to assess Ras cellular signaling in vitro, e.g., in response to a compound described herein. For example, ASPC-1 culture medium is prepared with RPMI-1640 and 10% heat-inactivated FBS. Panc-10.05 culture medium is prepared with RPMI-1640, 10 Units/mL human recombinant insulin, and 10% FBS. A427 cell culture is prepared with RPMI-1640 and 10% heat-inactivated FBS. A CellTiter-Glo (CTG) luminescent based assay (Promega) is used to assess growth of the cells, as a measurement of the ability of the compounds herein to inhibit Ras signaling in the cells. The cells (e.g., 800 per well) are seeded in their respective culture medium in standard tissue culture-treated 384-well format plates (Falcon #08-772-116) or ultra-low attachment surface 384-well format plates (S-Bio #MS-9384UZ). The day after plating, cells are treated with a dilution series (e.g., a 9 point, 3-fold dilution series) of the compounds herein (e.g., approximately 40 μL final volume per well). Cell viability can be monitored (e.g., approximately 5 days later) according to the manufacturer's recommended instructions, where CellTiter-Glo reagent is added (e.g., approximately 10 μL), vigorously mixed, covered, and placed on a plate shaker (e.g., approximately for 20 min) to ensure sufficient cell lysis prior to assessment of luminescent signal. The IC50 values are determined using the four-parameter fit. The resulting IC50 value is a measurement of the ability of the test compound to reduce cell growth of Ras-driven cells (e.g., tumor cell lines) in vitro and/or in vivo.

c. Growth of Cells with K-Ras G12S Mutation

A549 (ATCC CRL-185) and LS123 (ATCC CRL-255) cell lines comprise a G12S mutation and can be used to assess Ras cellular signaling in vitro, e.g., in response to treatment with a compound described herein. A549 culture medium is prepared with RPMI-1640 and 10% heat-inactivated FBS. LS123 culture medium is prepared with RPMI-1640 and 10% heat-inactivated FBS. A CellTiter-Glo (CTG) luminescent based assay (Promega) is used to assess growth of the cells, as a measurement of the ability of the compounds herein to inhibit Ras signaling in the cells. The cells (e.g., 800 per well) are seeded in their respective culture medium in standard tissue culture-treated 384-well format plates (Falcon #08-772-116) or ultra-low attachment surface 384-well format plates (S-Bio #MS-9384WZ). The day after plating, cells are treated with a dilution series (e.g., a 10 point 3-fold dilution series) of the compounds herein (e.g., approximately 40 μl final volume per well). Cell viability can be monitored (e.g., approximately 6 days later) according to the manufacturer's recommended instructions, where CellTiter-Glo reagent is added (e.g., approximately 10 μl), vigorously mixed, covered, and placed on a plate shaker (e.g., approximately for 20 min) to ensure sufficient cell lysis prior to assessment of luminescent signal. The IC50 values are determined using the four parameter fit. The resulting IC50 value is a measurement of the ability of the test compound to reduce cell growth of Ras-driven cells (e.g., tumor cell lines) in vitro and/or in vivo. The ability of one or more compounds exemplified in Table 1 to inhibit growth of one or more cell lines comprising a given Kras mutation is demonstrated utilizing the procedures described above. One or more compounds disclosed herein selectively inhibits growth of cells with a K-Ras G12S mutation compared to cells with a K-Ras G12D mutation. For example, compounds of the present disclosure are expected to selectively inhibit growth of K-Ras G12S mutant cells as evidenced by an IC50 against a K-Ras G12D cell line such as GP2D of greater than 1-fold, 2-fold, 3-fold, 4-fold, 5-fold, 10-fold, 15-fold, or even higher relative to an IC50 against a K-Ras G12S cell line such as A549 (i.e., the ratio of IC50 against a G12D cell line to IC50 against a G12S cell line is greater than 1, 2, 3, 4, 5, 10, 15 or even greater).

Table 3 shows the resulting IC50 values of exemplary compounds against A549 cells using the cell proliferation assays described herein. Compound numbers correspond to the numbers and structures provided in Table 1 and Example 1.

TABLE 3

| | ≤500 nM | 500 nM to 5 μM |
|---|---|---|
| Inhibition | 104, 108, 109, 111, 112, 115, 116, 117, | 105, 113, 121, 140, |
| of A549 | 118, 119, 120, 123, 124, 125, 127, 129, | 145, 149, 151, 153, |
| Cells | 130, 131, 133, 134, 135, 136, 137, 138, | 156, 166, 172, 183, |
| (IC₅₀) | 141, 142, 143, 144, 146, 147, 148, 150, | 188, 232 |
| | 152, 154, 160, 161, 162, 165, 171, 175, | |
| | 176, 177, 178, 179, 180, 181, 182, 184, | |
| | 185, 186, 190, 192, 193, 194, 198, 199, | |
| | 203, 204, 206, 208, 209, 210, 213, 216, | |
| | 219, 223, 224, 226, 227, 228, 229, 230, | |
| | 234, 235, 236, 237 | |

One problem in the art is that reversible compounds substantially lack the ability to bind to the serine residue at position 12 of K-Ras G12S mutant and inhibit K-Ras G12S signaling. The present disclosure provides warheads that are particularly suited for reacting with the serine residue at position 12 of K-Ras G12S mutant as a solution to this problem.

### Example 10: In Vivo Ras Inhibition

The in vivo reduction in Ras signaling output by a compound of the present disclosure is determined in a mouse tumor xenograft model, particularly by using a mutant K-Ras model including without limitation a K-Ras G12S model, a K-Ras G12C model, a K-Ras G12D model, a K-Ras G13D model, and a K-Ras G13C model. These models can be generated by the methods and procedures described below. In particular, the methods disclosed below involving the use of a K-Ras G12S mutant cell line for generating a K-Ras G12S xenograft model can be applied to other K-Ras mutant animal models using the respective K-Ras mutant cell lines described above.

Xenograft with K-Ras G12D, G12C, or G12S Mutation

Tumor xenografts are established by administration of tumor cells with a K-Ras G12D mutation (e.g., ASPC-1 cells), a K-Ras G12C mutation (e.g., MIA PaCa-2 cells), or a K-Ras G12S mutation (e.g., A549 or LS123 cells) into mice. Female 6- to 8-week-old athymic BALB/c nude (NCr) nu/nu mice are used for xenografts. The tumor cells (e.g., approximately 5×10⁶) are harvested on the day of use and injected in growth-factor-reduced Matrigel/PBS (e.g., 50% final concentration in 100 μL). One flank is inoculated subcutaneously per mouse. Mice are monitored daily, weighed twice weekly, and caliper measurements begin when tumors become visible. For efficacy studies, animals are randomly assigned to treatment groups by an algorithm that assigns animals to groups to achieve best case distributions of mean tumor size with lowest possible standard deviation. Tumor volume can be calculated by measuring two perpendicular diameters using the following formula: (L×w²)/2, in which L and w refer to the length and width of the tumor, respectively. Percent tumor volume change can be calculated using the following formula: (V_{final}−Vᵢₙᵢₜᵢₐₗ)/Vᵢₙᵢₜᵢₐₗ×100. Percent of tumor growth inhibition (% TGI) can be calculated using the following formula: % TGI=100×(1−(average V_{final}−Vᵢₙᵢₜᵢₐₗ of treatment group)/(average V_{final}−Vᵢₙᵢₜᵢₐₗ of control group). When tumors reach a threshold average size (e.g., approximately 200-400 mm3), mice are randomized into 3-10 mice per group and are treated with vehicle (e.g., 100% Labrasol®) or a compound disclosed herein, using, for example, a daily schedule by oral gavage. Results can be expressed as mean and standard deviation of the mean.

### Example 11: Metabolic (Microsomal) Stability Assay

The metabolic stability of a test compound is assayed at 37° C. using pooled liver microsomes (mouse or human liver microsomes). An aliquot of 10 μL of 50 μM test compound is mixed with 490 μL of 0.611 mg/mL liver microsomes, then 50 μL of the mixtures are dispensed to the 96 well tubes and warmed at 37° C. for 10 minutes. The reactions are initiated by adding 50 μL of the pre-warmed NADPH regeneration system solution (add 1.2 μL solution, 240 μl solution B, mix with 10.56 ml KPBS) and then incubated at 37° C. The final incubation solution contains 100 mM potassium phosphate (pH 7.4), 1.3 mM NADP+, 3.3 mM glucose 6-phosphate, 0.4 Unit/mL of glucose 6-phosphate dehydrogenase, 3.3 mM magnesium chloride, 0.3 mg/mL liver microsomes and 0.5 μM test article. After 0, 15, 30 and 60 minutes in a shaking incubator, the reactions are terminated by adding 100 μL of acetonitrile containing 200 nM buspirone as an internal standard. All incubations are conducted in duplicate. Plates are vortexed vigorously by using Fisher Scientific microplate vortex mixer (Henry Troemner, US). Samples are then centrifuged at 3500 rpm for 10 minutes (4° C.) using Sorvall Legend XRT Centrifuge (Thermo Scientific, GE). Supernatants (40 μL) are transferred into clean 96-deep well plates. To each well is added with 160 μL of ultrapure water (Milli-Q, Millipore Corporation) with 0.1% (v/v) formic acid (Fisher Chemical) and the resulting solutions mixed thoroughly and subjected to LC/MS/MS analysis in MRM positive ionization mode.

All the samples are measured using a mass spectrometer (QTrap 5500 quadrupole/ion trap) coupled with a Shimadzu HPLC system. The HPLC system consists of a Shimadzu series degasser, binary quaternary gradient pumps, column heater coupled to an autosampler, and a Phenomenex Gemini-NX, C18, 3.0 μm or Phenomenex Lunar, C8, 5.0 μM HPLC column (Phenomenex, Torrance, CA), eluting with a mobile phase gradient consisting of Solution A (0.1% formic acid water) and Solution B (0.1% formic acid acetonitrile). The column temperature is maintained at 40° C. All the analytes are detected with positive-mode electrospray ionization (ES+).

The half-life for the metabolic degradation of the test compound is calculated by plotting the time-course disappearance of the test compound during the incubation with liver microsomes. Each plot is fitted to a first-order equation for the elimination of the test compound (% remaining compound) versus time using non-linear regression (Equation 1).

$$\frac{{C}_{t}}{{C}_{0}} = {e}^{- k t} \qquad Equation 1$$

where Cₜ is the mean relative substrate concentration at time t and C₀ is the initial concentration (0.5 μM) at time 0. Note that the area ratio of the substrate peak to an internal standard peak is proportional to the analyte concentration and is used for regression analysis to derive a value of k.

The half-life t_{1/2} for metabolic (microsome) stability is derived from the test compound elimination constant k using Equation 2 below.

$${t}_{1 / 2} = \frac{0.693}{k} \qquad Equation 2$$

### Example 12: CYP2C19 Inhibition Assay

Some xenobiotics can inhibit cytochrome P450 (CYP) enzyme function, which alters their ability to metabolize drugs. Administration of a CYP inhibitor with a drug whose clearance is dependent on CYP metabolism can result in increased plasma concentrations of this concomitant drug, leading to potential toxicity. The inhibition of CYP2C19 by a test compound is assayed in human liver microsomes using S-Mephenytoin as a CYP2C19 substrate. The stock solution of the test compound or known CYP2C19 inhibitor as a positive control (10 mM) is diluted with KPBS to 40 μM. In a similar way, the stock solutions of the human liver microsomes and S-Mephenytoin are diluted with KPBS buffer. The pre-incubations are started by incubating a plate containing 25 μL human liver microsomes (final concentration of 0.2 mg/mL), 25 μL NADPH-generating system, and a 25 μL test compound (final concentration 10 μM) or the positive control for 30 min at 37±1° C. After the pre-incubation, 25 μL S-Mephenytoin (final concentration 200 μM) is added and incubated another 12 minutes at 37±1° C. for substrate metabolism. The reactions are terminated by addition of 100 μL of ice-cold acetonitrile containing an internal standard (buspirone). Precipitated proteins are removed by centrifugation at 3500 rpm for 10 minutes at 4° C. (Allegra 25R, Beckman Co. Fullerton, CA), then an aliquot of the supernatant is transferred to an assay plate.

All the samples are assessed using a mass spectrometer (QTrap 5500 quadrupole/ion trap) coupled with a Shimadzu HPLC system following the manufacturer's instructions. The metabolism of S-Mephenytoin in human liver microsomes is monitored by LC/MS/MS as representative of CYP2C19 inhibitory activity. The amount of metabolite formed is assessed by the peak area ratio (metabolite/IS) and % inhibition at 10 μM is expressed as a percentage of the metabolite signal reduced compared to the control (i.e. an incubation that contained no inhibitor and represented 100% enzyme activity): % inhibition=(1−A/B)×100%, where A is the metabolite peak area ratio formed in the presence of test compound or inhibitor at 10 μM and B is the metabolite peak area ratio formed without test compound or inhibitor in the incubation.

### Example 13: Mouse and Human Protein Binding Assay to Assess Free Drug Concentration

This assay can be used to determine the plasma protein binding of the test compound in the plasma of human and animal species using a Rapid Equilibrium Dialysis (RED) device for equilibrium dialysis and LC-MS/MS for sample analysis. Test compound is spiked in. The stock solution of the test compound is prepared at 5 mM concentration. One μL of 5 mM working solution is added into 1000 μL plasma to achieve a final concentration of 5 μM. The spiked plasma is placed on a rocker and gently agitated for approximately 20 minutes. A volume of 300 μL of the plasma sample containing 5 μM test compound from each species is added to designated RED device donor chambers followed by addition of 500 μL of potassium phosphate buffer to the corresponding receiver chambers in duplicate. The RED device is then sealed with sealing tape and shaken at 150 RPM for 4 hours at 37° C. Post-dialysis donor and receiver compartment samples are prepared for LC-MS/MS analysis, including spiking samples with an internal standard for the bioanalytical analysis. Warfarin and propranolol are purchased from Sigma-Aldrich (St. Louis, MO), and used as positive controls for low and high plasma protein binding, respectively.

All the samples are analyzed using an Agilent Technologies 6430 Triple Quad LC/MS system. The HPLC system consists of an Agilent 1290 Infinity Liquid Chromatograph coupled to an autosampler (Agilent 1290 Infinity LC Injector HTC), and a Phenomenex Gemini-NX, C18, 3.0 μm or Phenomenex Lunar, C8, 5.0 μM HPLC column (Phenomenex, Torrance, CA), eluting with a mobile phase gradient consisting of Solution A (0.1% formic acid water) and Solution B (0.1% formic acid acetonitrile). The column temperature is maintained at 40° C. All the analytes are detected with positive-mode electrospray ionization (ES+). The percentage of the test compound bound to plasma is calculated following Equations 3 and 4.

$$\% Free test compound = \frac{Peak ratio ( \frac{test compound}{Internal standard} ) , receiver compartment}{Peak ratio ( \frac{test compound}{Internal standard} ) , donor compartment} * 100 \qquad Equation 3$$

$$\% Plasma protein bound test compound = 100 - \% Free test compound \qquad Equation 4$$

### Example 14: HERG (Automated Patch-Clamp) Assay

The human ether-a-go-go related gene (hERG) encodes the voltage gated potassium channel in the heart (IKr) which is involved in cardiac repolarization. Inhibition of the hERG causes QT interval prolongation and can lead to potential fatal events in humans. It is thus important to assess hERG inhibition early in drug discovery. A hERG automated patch-clamp assay is done using a hERG CHO-K1 cell line using an incubation time of 5 min. The degree of hERG inhibition (%) is obtained by measuring the tail current amplitude, which is induced by a one second test pulse to −40 mV after a two second pulse to +20 mV, before and after drug incubation (the current difference is normalized to control and multiplied by 100 to obtain the percent of inhibition). The percent hERG inhibition is measured in the presence of 10 μM test compound.

### Example 15: Rat Oral Exposure (% F)

A pharmacokinetic profile for a test compound is measured by single dosing in jugular vein cannulated male Sprague-Dawley rats. Animal weights are typically over 200 grams, and animals are allowed to acclimate to their new environment for at least 3 days prior to the initiation of any studies. One set of animals is dosed intravenously (IV) with test compound (2 mg/kg in 20% HP-beta-CD or 20% Captisol, pH adjusted to ˜4 by citric acid). The IV dosing solution concentration is 0.4 mg/mL test compound. Blood is sampled at 5 minutes, 15 minutes, 30 minutes, 90 minutes, 360 minutes, and 24 hours following IV dosing. Another set of animals is dosed oral (po) with test compound (10 mg/kg in 20% HP-beta-CD or 20% Captisol, pH adjusted to ˜4 by citric acid). The oral dosing solution concentration is 1 mg/mL test compound. Blood is sampled at 15 minutes, 30 minutes, 90 minutes, 180 minutes, 360 minutes and 24 hours following oral (po) dosing. Blood samples (˜0.2 mL/sample) is collected via the jugular vein, placed in tubes containing EDTA-K2 and stored on ice until centrifuged. The blood samples are centrifuged at approximately 6800 g for 6 minutes at 2-8° C. and the resulting plasma is separated and stored frozen at approximately −80° C.

The plasma samples are analyzed using an Agilent Technologies 6430 Triple Quad LC/MS system, following the manufacturer's instructions. The analytes are detected with positive-mode electrospray ionization (ES+). A standard curve for each test compound is generated and used to measure test compound concentrations in the rat plasma samples. Based on the time course sampling, an area under the curve is calculated for the oral dose group and the intravenous dose group. Percentage rat bioavailability is calculated based on equation 5.

$$\% F ( rat ) = \frac{A U {C}_{po} * {Dose}_{IV}}{A U {C}_{IV} * {Dose}_{po}} , \qquad Equation 5$$

where F is bioavailability, AUCₚₒ is area under curve of oral drug, AUC_{IV} is area under curve of intravenous drug, Dose_{IV} is the intravenous dose and Doseₚₒ is the oral dose.

1. A modified Ras protein comprising a compound covalently bonded to one or more amino acid residues of said Ras protein, wherein the modified Ras protein comprises a compound of Formula (I′): 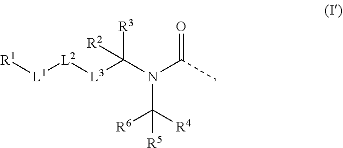
wherein:
the dashed line represents the covalent bond to the amino acid residue:
R¹ is selected from C₃₋₁₂ carbocycle and 3- to 12-membered heterocycle, each of which is optionally substituted with one or more R²⁰:
L¹ is 5- to 20-membered heterocycle optionally substituted with one or more R²⁰;
L² is selected from a bond, C₁₋₄ alkylene, 2- to 4-membered heteroalkylene, —O—, —N(R¹⁹)—, —C(O)—, —S—, —S(O)₂—, —S(O)—, —P(O)R¹⁹—, —N(R¹⁹)S(O)₂—, —N(R¹⁹)S(O)—, —N(R¹⁹)P(O)R¹⁹—, —S(O)₂N(R¹⁹)—, —S(O)N(R¹⁹)—, —P(O)R¹⁹N(R¹⁹)—, —OS(O)₂—, —OS(O)—, —OP(O)R¹⁹—, —S(O)₂O—, —S(O)O—, and —P(O)R¹⁹O—, wherein C₁₋₄ alkylene and 2- to 4-membered heteroalkylene are optionally substituted with one, two, or three R²⁰;
L³ and R², together with the atoms to which they are attached, form 3- to 8-membered monocyclic heterocycloalkyl, C₃₋₈ monocyclic cycloalkyl, 7- to 12-membered spirocyclic heterocycloalkyl, or 7- to 12-membered fused bicyclic heterocycloalkyl, each of which is optionally substituted with one, two, or three substituents selected from halogen, —OH, —CN, C₁₋₆ alkyl, C₃₋₆ cycloalkyl, —O(C₁₋₆ alkyl), and —O(C₃₋₆ cycloalkyl), wherein C₁₋₆ alkyl, C₃₋₆ cycloalkyl, —O(C₁₋₆ alkyl), and —O(C₃₋₆ cycloalkyl) are optionally substituted with one, two, or three substituents selected from halogen, —OH, —CN, C₁₋₃ alkyl, C₁₋₃ haloalkyl, C₃₋₆ cycloalkyl, —O(C₁₋₃ alkyl), and —O(C₁₋₃ haloalkyl);
L³ and R⁶, together with the atoms to which they are attached, form 4- to 8-membered monocyclic heterocycloalkyl, 7- to 12-membered spirocyclic heterocycloalkyl, or 7- to 12-membered fused bicyclic heterocycloalkyl, each of which is optionally substituted with one, two, or three substituents selected from halogen, —OH, —CN, C₁₋₆ alkyl, C₃₋₆ cycloalkyl, —O(C₁₋₆ alkyl), and —O(C₃₋₆ cycloalkyl), wherein C₁₋₆ alkyl, C₃₋₆ cycloalkyl, —O(C₁₋₆ alkyl), and —O(C₃₋₆ cycloalkyl) are optionally substituted with one, two, or three substituents selected from halogen, —OH, —CN, C₁₋₃ alkyl, C₁₋₃ haloalkyl, C₃₋₆ cycloalkyl, —O(C₁₋₃ alkyl), and —O(C₁₋₃ haloalkyl), and wherein the 4- to 8-membered monocyclic heterocycloalkyl formed by L³ and R⁶ is not piperazine; or
R² and R⁶, together with the atoms to which they are attached, form 3- to 8-membered monocyclic heterocycloalkyl optionally substituted with one, two, or three substituents selected from halogen, —OH, —CN, C₁₋₆ alkyl, C₃₋₆ cycloalkyl, —O(C₁₋₆ alkyl), and —O(C₃₋₆ cycloalkyl), wherein C₁₋₆ alkyl, C₃₋₆ cycloalkyl, —O(C₁₋₆ alkyl), and —O(C₃₋₆ cycloalkyl) are optionally substituted with one, two, or three substituents selected from halogen, —OH, —CN, C₁₋₃ alkyl, C₁₋₃ haloalkyl, C₃₋₆ cycloalkyl, —O(C₁₋₃ alkyl), and —O(C₁₋₃ haloalkyl), and wherein L³ is a bond:
R² is selected from halogen, —CN, C₁₋₆ alkyl, and C₃₋₆ cycloalkyl, or R² and R³, together with the carbon atom to which they are attached, form C₃₋₆ cycloalkyl, wherein C₁₋₆ alkyl and C₃₋₆ cycloalkyl are optionally substituted with one, two, or three substituents selected from halogen, —CN, C₁₋₆ alkyl, C₁₋₆ haloalkyl, C₃₋₆ cycloalkyl, —O(C₁₋₆ alkyl), and —O(C₁₋₆ haloalkyl);
R³ is selected from hydrogen, halogen, —CN, C₁₋₆ alkyl, and C₃₋₆ cycloalkyl, wherein C₁₋₆ alkyl and C₃₋₆ cycloalkyl are optionally substituted with one, two, or three substituents selected from halogen, —CN, C₁₋₆ alkyl, C₁₋₆ haloalkyl, C₃₋₆ cycloalkyl, —O(C₁₋₆ alkyl), and —O(C₁₋₆ haloalkyl);
R⁴, R⁵, and R⁶ are each independently selected from hydrogen, halogen, —CN, C₁₋₆ alkyl, and C₃₋₆ cycloalkyl, or R⁴ and R⁵, together with the carbon atom to which they are attached, form C₃₋₆ cycloalkyl, wherein C₁₋₆ alkyl and C₃₋₆ cycloalkyl are optionally substituted with one, two, or three substituents selected from halogen, —CN, C₁₋₆ alkyl, C₁₋₆ haloalkyl, C₃₋₆ cycloalkyl, —O(C₁₋₆ alkyl), and —O(C₁₋₆ haloalkyl);
R¹⁹ is independently selected at each occurrence from hydrogen, —CN, C₁₋₆ alkyl, C₃₋₆ carbocycle, 3- to 6-membered heterocycle, —OH, —O(C₁₋₆ alkyl), and —O(C₁₋₆ haloalkyl);
R²⁰ is independently selected at each occurrence from halogen, oxo, —CN, C₁₋₆ alkyl, C₂₋₆ alkenyl, C₂₋₆ alkynyl, 2- to 6-membered heteroalkyl, 2- to 6-membered heteroalkenyl, 2- to 6-membered heteroalkynyl, —C₀₋₆ alkyl-(C₃₋₁₂ carbocycle), -(2- to 6-membered heteroalkyl)-(C₃₋₁₂ carbocycle), —C₀₋₆ alkyl-(3- to 12-membered heterocycle), -(2- to 6-membered heteroalkyl)-(3- to 12-membered heterocycle), —OR²², —SR²², —N(R²²)(R²³), ═NR²², ═C(R²¹)₂, —C(O)OR²², —OC(O)N(R²²)(R²³), —N(R²²)C(O)N(R²²)(R²³), —N(R²²)C(O)OR²², —N(R²²)S(O)₂R²², —C(O)R²², —S(O)R²², —OC(O)R²², —C(O)N(R²²)(R²³), —C(O)C(O)N(R²²)(R²³), —N(R²²)C(O)R²², —S(O)₂R²², —S(O)(NR²²)R²², —S(O)₂N(R²²)(R²³)—, —S(═O)(═NR²²)N(R²²)(R²³), and —OCH₂C(O)OR²²; wherein two R²⁰ attached to the same or adjacent atoms optionally join to form C₃₋₁₂ carbocycle or 3- to 12-membered heterocycle; wherein C₁₋₆ alkyl, C₂₋₆ alkenyl, C₂₋₆ alkynyl, 2- to 6-membered heteroalkyl, 2- to 6-membered heteroalkenyl, 2- to 6-membered heteroalkynyl, —C₀₋₆ alkyl-(C₃₋₁₂ carbocycle), -(2- to 6-membered heteroalkyl)-(C₃₋₁₂ carbocycle), —C₀₋₆ alkyl-(3- to 12-membered heterocycle), -(2- to 6-membered heteroalkyl)-(3- to 12-membered heterocycle), C₃₋₁₂ carbocycle, and 3- to 12-membered heterocycle are optionally substituted with one or more substituents independently selected from halogen, oxo, —CN, C₁₋₆ alkyl, C₁₋₆ haloalkyl, C₁₋₆ alkoxy, C₁₋₆ haloalkoxy, —OR²², —SR²², —N(R²²)(R²³), —NR²², ═C(R²¹), —C(O)OR²², —OC(O)N(R²²)(R²³), —N(R²²)C(O)N(R²²)(R²³), —N(R²²)C(O)OR²², —N(R²²)S(O)₂R²², —C(O)R²², —S(O)R²², —OC(O)R²², —C(O)N(R²²)(R²³), —C(O)C(O)N(R²²)(R²³), —N(R²²)C(O)R²², —S(O)₂R²², —S(O)(NR²²)R²², —S(O)₂N(R²²)(R²³), and —S(═O)(═NR²²)N(R²²)(R²³);
R²¹ is independently selected at each occurrence from hydrogen, halogen, C₁₋₆ alkyl, C₁₋₆ haloalkyl, —C₀₋₆ alkyl-(C₃₋₁₂ carbocycle), and —C₀₋₆ alkyl-(3- to 12-membered heterocycle), or two R²¹ are taken together with the carbon atom to which they are attached to form C₃₋₁₂ carbocycle or 3- to 12-membered heterocycle, each of which is optionally substituted with one, two, or three substituents independently selected from halogen, C₁₋₃ alkyl, C₁₋₃ haloalkyl, and —OH;
R²² is independently selected at each occurrence from hydrogen, C₁₋₆ alkyl, C₁₋₆ haloalkyl, —C₀₋₆ alkyl-(C₃₋₁₂ carbocycle), and —C₀₋₆ alkyl-(3- to 12-membered heterocycle); and
R²³ is independently selected at each occurrence from hydrogen and C₁₋₆ alkyl; or R²² and R²³ attached to the same nitrogen atom form 3- to 10 membered heterocycle.

2-13. (canceled)

14. A compound of Formula (II): 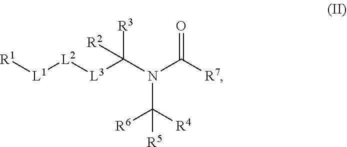
or a pharmaceutically acceptable salt or solvate thereof, wherein:
R¹ is selected from C₃₋₁₂ carbocycle and 3- to 12-membered heterocycle, each of which is optionally substituted with one or more R²⁰;
L¹ is 5- to 20-membered heterocycle optionally substituted with one or more R²⁰;
L² is selected from a bond, C₁₋₄ alkylene, 2- to 4-membered heteroalkylene, —O—, —N(R¹⁹)—, —C(O)—, —S—, —S(O)₂—, —S(O)—, —P(O)R¹⁹—, —N(R¹⁹)S(O)₂—, —N(R¹⁹)S(O)—, —N(R¹⁹)P(O)R¹⁹—, —S(O)₂N(R¹⁹)—, —S(O)N(R¹⁹)—, —P(O)R¹⁹N(R¹⁹)—, —OS(O)₂—, —OS(O)—, —OP(O)R¹⁹—, —S(O)₂O—, —S(O)O—, and —P(O)R¹⁹O—, wherein C₁₋₄ alkylene and 2- to 4-membered heteroalkylene are optionally substituted with one, two, or three R²⁰;
L³ and R², together with the atoms to which they are attached, form 3- to 8-membered monocyclic heterocycloalkyl, C₃₋₈ monocyclic cycloalkyl, 7- to 12-membered spirocyclic heterocycloalkyl, or 7- to 12-membered fused bicyclic heterocycloalkyl, each of which is optionally substituted with one, two, or three substituents selected from halogen, —OH, —CN, C₁₋₆ alkyl, C₃₋₆ cycloalkyl, —O(C₁₋₆ alkyl), and —O(C₃₋₆ cycloalkyl), wherein C₁₋₆ alkyl, C₃₋₆ cycloalkyl, —O(C₁₋₆ alkyl), and —O(C₃₋₆ cycloalkyl) are optionally substituted with one, two, or three substituents selected from halogen, —OH, —CN, C₁₋₃ alkyl, C₁₋₃ haloalkyl, C₃₋₆ cycloalkyl, —O(C₁₋₃ alkyl), and —O(C₁₋₃ haloalkyl);
L³ and R₆, together with the atoms to which they are attached, form 4- to 8-membered monocyclic heterocycloalkyl, 7- to 12-membered spirocyclic heterocycloalkyl, or 7- to 12-membered fused bicyclic heterocycloalkyl, each of which is optionally substituted with one, two, or three substituents selected from halogen, —OH, —CN, C₁₋₆ alkyl, C₃₋₆ cycloalkyl, —O(C₁₋₆ alkyl), and —O(C₃₋₆ cycloalkyl), wherein C₁₋₆ alkyl, C₃₋₆ cycloalkyl, —O(C₁₋₆ alkyl), and —O(C₃₋₆ cycloalkyl) are optionally substituted with one, two, or three substituents selected from halogen, —OH, —CN, C₁₋₃ alkyl, C₁₋₃ haloalkyl, C₃₋₆ cycloalkyl, —O(C₁₋₃ alkyl), and —O(C₁₋₃ haloalkyl), and wherein the 4- to 8-membered monocyclic heterocycloalkyl formed by L³ and R⁶ is not piperazine; or
R² and R⁶, together with the atoms to which they are attached, form 3- to 8-membered monocyclic heterocycloalkyl optionally substituted with one, two, or three substituents selected from halogen, —OH, —CN, C₁₋₆ alkyl, C₃₋₆ cycloalkyl, —O(C₁₋₆ alkyl), and —O(C₃₋₆ cycloalkyl), wherein C₁₋₆ alkyl, C₃₋₆ cycloalkyl, —O(C₁₋₆ alkyl), and —O(C₃₋₆ cycloalkyl) are optionally substituted with one, two, or three substituents selected from halogen, —OH, —CN, C₁₋₃ alkyl, C₁₋₃ haloalkyl, C₃₋₆ cycloalkyl, —O(C₁₋₃ alkyl), and —O(C₁₋₃ haloalkyl), and wherein L³ is a bond;
R² is selected from halogen, —CN, C₁₋₆ alkyl, and C₃₋₆ cycloalkyl, or R² and R³, together with the carbon atom to which they are attached, form C₃₋₆ cycloalkyl, wherein C₁₋₆ alkyl and C₃₋₆ cycloalkyl are optionally substituted with one, two, or three substituents selected from halogen, —CN, C₁₋₆ alkyl, C₁₋₆ haloalkyl, C₃₋₆ cycloalkyl, —O(C₁₋₆ alkyl), and —O(C₁₋₆ haloalkyl);
R³ is selected from hydrogen, halogen, —CN, C₁₋₆ alkyl, and C₃₋₆ cycloalkyl, wherein C₁₋₆ alkyl and C₃₋₆ cycloalkyl are optionally substituted with one, two, or three substituents selected from halogen, —CN, C₁₋₆ alkyl, C₁₋₆ haloalkyl, C₃₋₆ cycloalkyl, —O(C₁₋₆ alkyl), and —O(C₁₋₆ haloalkyl);
R⁴, R⁵, and R⁶ are each independently selected from hydrogen, halogen, —CN, C₁₋₆ alkyl, and C₃₋₆ cycloalkyl, or R⁴ and R⁵, together with the carbon atom to which they are attached, form C₃₋₆ cycloalkyl, wherein C₁₋₆ alkyl and C₃₋₆ cycloalkyl are optionally substituted with one, two, or three substituents selected from halogen, —CN, C₁₋₆ alkyl, C₁₋₆ haloalkyl, C₃₋₆ cycloalkyl, —O(C₁₋₆ alkyl), and —O(C₁₋₆ haloalkyl);
R⁷ is selected from 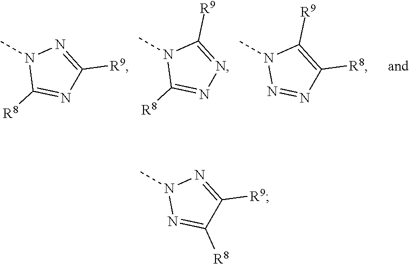
R⁸ and R⁹ are each independently selected from hydrogen, halogen, —CN, C₁₋₆ alkyl, and C₃₋₆ cycloalkyl, wherein C₁₋₆ alkyl and C₃₋₆ cycloalkyl are optionally substituted with one, two, or three substituents selected from halogen, —CN, C₁₋₆ alkyl, —O(C₁₋₆ alkyl), and —O(C₁₋₆ haloalkyl);
R¹⁹ is independently selected at each occurrence from hydrogen, —CN, C₁₋₆ alkyl, C₃₋₆ carbocycle, 3- to 6-membered heterocycle, —OH, —O(C₁₋₆ alkyl), and —O(C₁₋₆ haloalkyl);
R²⁰ is independently selected at each occurrence from halogen, oxo, —CN, C₁₋₆ alkyl, C₂₋₆ alkenyl, C₂₋₆ alkynyl, 2- to 6-membered heteroalkyl, 2- to 6-membered heteroalkenyl, 2- to 6-membered heteroalkynyl, —C₀₋₆ alkyl-(C₃₋₁₂ carbocycle), -(2- to 6-membered heteroalkyl)-(C₃₋₁₂ carbocycle), —C₀₋₆ alkyl-(3- to 12-membered heterocycle), -(2- to 6-membered heteroalkyl)-(3- to 12-membered heterocycle), —OR²², —SR²², —N(R²²)(R²³), ═NR²², ═C(R²¹)₂, —C(O)OR²², —OC(O)N(R²²)(R²³), —N(R²²)C(O)N(R²²)(R²³), —N(R²²)C(O)OR²², —N(R²²)S(O)₂R²², —C(O)R²², —S(O)R²², —OC(O)R²², —C(O)N(R²²)(R²³), —C(O)C(O)N(R²²)(R²³), —N(R²²)C(O)R²², —S(O)₂R²², —S(O)(NR²²)R²², —S(O)₂N(R²²)(R²³)—, —S(═O)(═NR²²)N(R²²)(R²³), and —OCH₂C(O)OR²²; wherein two R²⁰ attached to the same or adjacent atoms optionally join to form C₃₋₁₂ carbocycle or 3- to 12-membered heterocycle; wherein C₁₋₆ alkyl, C₂₋₆ alkenyl, C₂₋₆ alkynyl, 2- to 6-membered heteroalkyl, 2- to 6-membered heteroalkenyl, 2- to 6-membered heteroalkynyl, —C₀₋₆ alkyl-(C₃₋₁₂ carbocycle), -(2- to 6-membered heteroalkyl)-(C₃₋₁₂ carbocycle), —C₀₋₆ alkyl-(3- to 12-membered heterocycle), -(2- to 6-membered heteroalkyl)-(3- to 12-membered heterocycle), C₃₋₁₂ carbocycle, and 3- to 12-membered heterocycle are optionally substituted with one or more substituents independently selected from halogen, oxo, —CN, C₁₋₆ alkyl, C₁₋₆ haloalkyl, C₁₋₆ alkoxy, C₁₋₆ haloalkoxy, —OR²², —SR²², —N(R²²)(R²³), ═NR²², ═C(R²¹)₂, —C(O)OR²², —OC(O)N(R²²)(R²³), —N(R²²)C(O)N(R²²)(R²³), —N(R²²)C(O)OR²², —N(R²²)S(O)₂R²², —C(O)R²², —S(O)R²², —OC(O)R²², —C(O)N(R²²)(R²³), —C(O)C(O)N(R²²)(R²³), —N(R²²)C(O)R²², —S(O)₂R²², —S(O)(NR²²)R²², —S(O)₂N(R²²)(R²³), and —S(═O)(═NR²²)N(R²²)(R²³);
R²¹ is independently selected at each occurrence from hydrogen, halogen, C₁₋₆ alkyl, C₁₋₆ haloalkyl, —C₀₋₆ alkyl-(C₃₋₁₂ carbocycle), and —C₀₋₆ alkyl-(3- to 12-membered heterocycle), or two R²¹ are taken together with the carbon atom to which they are attached to form C₃₋₁₂ carbocycle or 3- to 12-membered heterocycle, each of which is optionally substituted with one, two, or three substituents independently selected from halogen, C₁₋₃ alkyl, C₁₋₃ haloalkyl, and —OH;
R²² is independently selected at each occurrence from hydrogen, C₁₋₆ alkyl, C₁₋₆ haloalkyl, —C₀₋₆ alkyl-(C₃₋₁₂ carbocycle), and —C₀₋₆ alkyl-(3- to 12-membered heterocycle); and
R²³ is independently selected at each occurrence from hydrogen and C₁₋₆ alkyl; or R²² and R²³ attached to the same nitrogen atom form 3- to 10 membered heterocycle.

15. The compound, salt, or solvate of claim 14, wherein L³ and R², together with the atoms to which they are attached, form 3- to 8-membered monocyclic heterocycloalkyl, C₃₋₈ monocyclic cycloalkyl, 7- to 12-membered spirocyclic heterocycloalkyl, or 7- to 12-membered fused bicyclic heterocycloalkyl, each of which is optionally substituted with one, two, or three substituents selected from halogen, —OH, —CN, C₁₋₆ alkyl, C₃₋₆ cycloalkyl, —O(C₁₋₆ alkyl), and —O(C₃₋₆ cycloalkyl), wherein C₁₋₆ alkyl, C₃₋₆ cycloalkyl, —O(C₁₋₆ alkyl), and —O(C₃₋₆ cycloalkyl) are optionally substituted with one, two, or three substituents selected from halogen, —OH, —CN, C₁₋₃ alkyl, C₁₋₃ haloalkyl, C₃₋₆ cycloalkyl, —O(C₁₋₃ alkyl), and —O(C₁₋₃ haloalkyl).

16. (canceled)

17. The compound, salt, or solvate of claim 14, wherein L³ and R⁶, together with the atoms to which they are attached, form 4- to 8-membered monocyclic heterocycloalkyl, 7- to 12-membered spirocyclic heterocycloalkyl, or 7- to 12-membered fused bicyclic heterocycloalkyl, each of which is optionally substituted with one, two, or three substituents selected from halogen, —OH, —CN, C₁₋₆ alkyl, C₃₋₆ cycloalkyl, —O(C₁₋₆ alkyl), and —O(C₃₋₆ cycloalkyl), wherein C₁₋₆ alkyl, C₃₋₆ cycloalkyl, —O(C₁₋₆ alkyl), and —O(C₃₋₆ cycloalkyl) are optionally substituted with one, two, or three substituents selected from halogen, —OH, —CN, C₁₋₃ alkyl, C₁₋₃ haloalkyl, C₃₋₆ cycloalkyl, —O(C₁₋₃ alkyl), and —O(C₁₋₃ haloalkyl), wherein the 4- to 8-membered monocyclic heterocycloalkyl formed by L³ and R⁶ is not piperazine.

18. The compound, salt, or solvate of claim 14, wherein R² and R⁶, together with the atoms to which they are attached, form 3- to 8-membered monocyclic heterocycloalkyl optionally substituted with one, two, or three substituents selected from halogen, —OH, —CN, C₁₋₆ alkyl, C₃₋₆ cycloalkyl, —O(C₁₋₆ alkyl), and —O(C₃₋₆ cycloalkyl), wherein C₁₋₆ alkyl, C₃₋₆ cycloalkyl, —O(C₁₋₆ alkyl), and —O(C₃₋₆ cycloalkyl) are optionally substituted with one, two, or three substituents selected from halogen, —OH, —CN, C₁₋₃ alkyl, C₁₋₃ haloalkyl, C₃₋₆ cycloalkyl, —O(C₁₋₃ alkyl), and —O(C₁₋₃ haloalkyl), wherein L³ is a bond.

19-44. (canceled)

45. The compound, salt, or solvate of claim 14, wherein R¹ is selected from naphthyl, isoquinolinyl, indazolyl, benzothiazolyl, benzothiophenyl, phenyl, and pyridinyl, each of which is optionally substituted with one or more R²⁰.

46-49. (canceled)

50. The compound, salt, or solvate of claim 14, wherein R¹ is

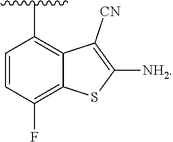 [SMILES: *c1ccc(F)c2sc(N)c(C#N)c12]

51. (canceled)

52. The compound, salt, or solvate of claim 14, wherein L¹ is 10-membered bicyclic heterocycle substituted with one, two, three, or four R²⁰.

53. The compound, salt, or solvate of claim 14, wherein L¹ is selected from pyridopyrimidine and quinazoline.

54-70. (canceled)

71. The compound, salt, or solvate of claim 14, wherein R² is selected from C₁₋₆ alkyl and C₃₋₆ cycloalkyl.

72. The compound, salt, or solvate of claim 14, wherein R³ is selected from hydrogen and C₁₋₆ alkyl.

73. (canceled)

74. (canceled)

75. The compound, salt, or solvate of claim 14, wherein R₇ is

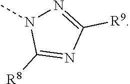

76. The compound, salt, or solvate of claim 14, wherein R⁸ is selected from hydrogen, halogen, —CH₃, —CH₂F, —CHF₂, and —CF₃.

77. (canceled)

78. The compound, salt, or solvate of claim 14, wherein R⁹ is selected from hydrogen, halogen, —CH₃, —CH₂F, —CHF₂, and —CF₃.

79. (canceled)

80. The compound, salt, or solvate of claim 75, wherein R₇ is

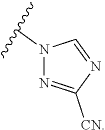 [SMILES: *n1cnc(C#N)n1]

81. The compound, salt, or solvate of claim 14, wherein:
R¹ is selected from naphthyl, isoquinolinyl, indazolyl, benzothiazolyl, benzothiophenyl, phenyl, and pyridinyl, each of which is optionally substituted with one or more R²⁰;
L¹ is 10-membered bicyclic heterocycle substituted with one, two, three, or four R²⁰;
L² is selected from a bond, C₁₋₃ alkylene, and 2- to 3-membered heteroalkylene, wherein C₁₋₃ alkylene and 2- to 3-membered heteroalkylene are optionally substituted with one, two, or three R²⁰; and
R⁷ is

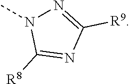

82-84. (canceled)

85. A pharmaceutical composition comprising a compound of claim 14, or a pharmaceutically acceptable salt or solvate thereof, and a pharmaceutically acceptable excipient.

86. A method of modifying a Ras mutant protein, comprising contacting the Ras mutant protein with an effective amount of the compound, salt, or solvate of claim 14.

87-96. (canceled)

97. A method of treating cancer comprising a K-Ras G12S mutant protein in a subject in need thereof, comprising administering to the subject a therapeutically effective amount of a compound of claim 14, or a pharmaceutically acceptable salt or solvate thereof.

98-102. (canceled)

103. The method of claim 97, comprising administering an additional agent.

104-111. (canceled)


SEQUENCE LISTING (13 sequences)

SEQ ID NO: 1 - protein, 188 aa, Homo sapiens [sequence omitted]

SEQ ID NO: 2 - protein, 188 aa, Homo sapiens [sequence omitted]

SEQ ID NO: 3 - protein, 188 aa, Homo sapiens
MTEYKLVVVGAVGVGKSALTIQLIQNHFVDEYDPTIEDSYRKQVVIDGETCLLDILDTAGQEEYSAMRDQYMRTGEGFLCVFAINNTKSFEDIHHYREQIKRVKDSEDVPMVLVGNKCDLPSRTVDTKQAQDLARSYGIPFIETSAKTRQGVDDAFYTLVREIRKHKEKMSKDGKKKKKKSKTKCVIM

SEQ ID NO: 4 - protein, 188 aa, Homo sapiens [sequence omitted]

SEQ ID NO: 5 - protein, 189 aa, Homo sapiens
MTEYKLVVVGAGGVGKSALTIQLIQNHFVDEYDPTIEDSYRKQVVIDGETCLLDILDTAGQEEYSAMRDQYMRTGEGFLCVFAINNSKSFADINLYREQIKRVKDSDDVPMVLVGNKCDLPTRTVDTKQAHELAKSYGIPFIETSAKTRQGVEDAFYTLVREIRQYRMKKLNSSDDGTQGCMGLPCVVM

SEQ ID NO: 6 - protein, 189 aa, Homo sapiens
MTEYKLVVVGADGVGKSALTIQLIQNHFVDEYDPTIEDSYRKQVVIDGETCLLDILDTAGQEEYSAMRDQYMRTGEGFLCVFAINNTKSFEDIHQYREQIKRVKDSDDVPMVLVGNKCDLAARTVESRQAQDLARSYGIPYIETSAKTRQGVEDAFYTLVREIRQHKLRKLNPPDESGPGCMSCKCVLS

SEQ ID NO: 7 - protein, 189 aa, Homo sapiens
MTEYKLVVVGAGGVGKSALTIQLIQNHFVDEYDPTIEDSYRKQVVIDGETCLLDILDTAGQEEYSAMRDQYMRTGEGFLCVFAINNTKSFEDIHQYREQIKRVKDSDDVPMVLVGNKCDLAARTVESRQAQDLARSYGIPYIETSAKTRQGVEDAFYTLVREIRQHKLRKLNPPDESGPGCMSCKCVLS

SEQ ID NO: 8 - protein, 189 aa, Homo sapiens [sequence omitted]

SEQ ID NO: 9 - protein, 108 aa, Homo sapiens
MTEYKLVVVGAGGVGKSALTIQLIQNHFVDEYDPTIEDSYRKQVVIDGETCLLDILDTAGQEEYSAMRDQYMRTGEGFLCVFAINNTKSFEDIHHYREQIKRVKDSED

SEQ ID NO: 10 - protein, 108 aa, Homo sapiens
MTEYKLVVVGAGGVGKSALTIQLIQNHFVDEYDPTIEDSYRKQVVIDGETCLLDILDTAGQEEYSAMRDQYMRTGEGFLCVFAINNTKSFEDIHQYREQIKRVKDSDD

SEQ ID NO: 11 - protein, 108 aa, Homo sapiens
MTEYKLVVVGAGGVGKSALTIQLIQNHFVDEYDPTIEDSYRKQVVIDGETCLLDILDTAGQEEYSAMRDQYMRTGEGFLCVFAINNSKSFADINLYREQIKRVKDSDD

SEQ ID NO: 12 - protein, 119 aa, Homo sapiens
MAANKPKGQNSLALHKVIMVGSGGVGKSALTLQFMYDEFVEDYEPTKADSYRKKVVLDGEEVQIDILDTAGQEDYAAIRDNYFRSGEGFLCVFSITEMESFAATADFREQILRVKEDEN

SEQ ID NO: 13 - protein, 120 aa, Homo sapiens
MAANKSKGQSSLALHKVIMVGSGGVGKSALTLQFMYDEFVEDYEPTKADSYRKKVVLDGEEVQIDILDTAGQEDYAAIRDNYFRSGEGFLLVFSITEHESFTATAEFREQILRVKAEEDK